(19) United States
(12) Patent Application Publication
(10) Pub. No.: US 2026/0297107 A1
(43) Pub. Date: Oct. 1, 2026

(54) ORGANIC ELECTROLUMINESCENT ELEMENT AND ELECTRONIC DEVICE

(71) Applicant: IDEMITSU KOSAN CO., LTD., Tokyo (JP)

(72) Inventors: Satomi TASAKI, Tokyo (JP); Kazuki NISHIMURA, Tokyo (JP); Ryoji MAEDA, Tokyo (JP); Yuichiro KAWAMURA, Tokyo (JP); Yuki NAKANO, Tokyo (JP)

(73) Assignee: IDEMITSU KOSAN CO., LTD., Tokyo (JP)

(21) Appl. No.: 19/529,626

(22) Filed: Feb. 4, 2026

Related U.S. Application Data
(63) Continuation of application No. 17/642,220, filed on Sep. 11, 2020, now Pat. No. 12,583,864.

(30) Foreign Application Priority Data
Sep. 13, 2019 (JP) 2019-167582
Nov. 26, 2019 (JP) 2019-213461
Dec. 27, 2019 (JP) 2019-239933
Apr. 1, 2020 (JP) 2020-066268
Apr. 15, 2020 (JP) 2020-073061

(51) Int. Cl. C07D 493/04; C07C 13/62; C07C 15/28; C07C 15/30; C07C 15/38; C07C 255/34; C07C 255/51; C07D 211/54; C07D 231/56; C07D 241/38; C07D 251/24; C07D 307/91; C07D 311/82; C07D 333/76; C07D 403/10; C07D 403/14; C07D 405/10; C07D 405/12; C07D 471/06; C07D 487/06; C07D 491/04; C07D 491/16; C07D 491/22; C07D 495/04; C07D 495/22; C07D 519/00; C07F 1/02; C07F 5/02; C07F 7/08; H10K 50/11; H10K 85/40; H10K 85/60; H10K 101/10

(52) U.S. Cl. CPC C07D 493/04; C07C 13/62; C07C 15/28; C07C 15/30; C07C 15/38; C07C 255/34; C07C 255/51; C07D 211/54; C07D 231/56; C07D 241/38; C07D 251/24; C07D 307/91; C07D 311/82; C07D 333/76; C07D 403/10; C07D 403/14; C07D 405/10; C07D 405/12; C07D 471/06; C07D 487/06; C07D 491/04; C07D 491/16; C07D 491/22; C07D 495/04; C07D 495/22; C07D 519/00; C07F 1/02; C07F 5/027; C07F 7/081; C07F 7/0812; C07F 7/0816; H10K 50/11; H10K 85/40; H10K 85/615; H10K 85/622; H10K 85/623; H10K 85/624; H10K 85/631; H10K 85/633; H10K 85/636; H10K 85/652; H10K 85/654; H10K 85/657; H10K 85/6572; H10K 85/6574; H10K 85/6576; H10K 85/658; C07B 2200/05; H10K 2101/10

(57) ABSTRACT

An organic EL device includes a first emitting layer and a second emitting layer, in which the first emitting layer contains a first host material, the second emitting layer contains a second host material, the first and the second host material are mutually different, the first emitting layer and the second emitting layer at least contains a compound that emits fluorescence having a main peak wavelength of 500 nm or less, respectively, the compound contained in the first emitting layer and that emits fluorescence having a main peak wavelength of 500 nm or less is the same as or different from the compound contained in the second emitting layer, and a triplet energy T₁(H1) of the first host material and a triplet energy T₁(H2) of the second host material satisfy a numerical formula (Numerical Formula 1),

$${T}_{1} ( H 1 ) > {T}_{1} ( H 2 ) . \qquad ( Numerical Formula 1 )$$

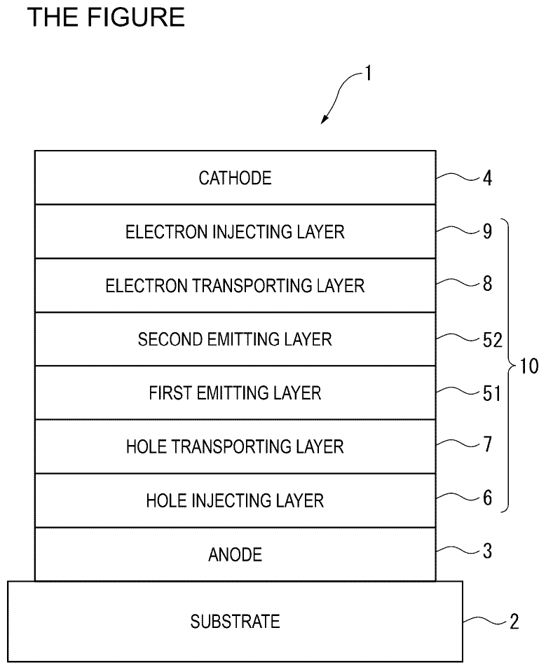

### CROSS-REFERENCE TO RELATED APPLICATIONS

The present application is a Continuation of U.S. patent application Ser. No. 17/642,220, filed on Sep. 11, 2020, which claims priority under 35 U.S.C. § 371 to International Patent Application No. PCT/JP2020/034593, filed Sep. 11, 2020, which claims priority to and the benefit of Japanese Patent Application Nos. 2019-167582, filed on Sep. 13, 2019, 2019-213461, filed on Nov. 26, 2019, 2019-239933, filed on Dec. 27, 2019, 2020-066268, filed on Apr. 1, 2020, and 2020-073061, filed on Apr. 15, 2020. The contents of these applications are hereby incorporated by reference in their entireties.

### TECHNICAL FIELD

The present invention relates to an organic electroluminescence device and an electronic device.

### BACKGROUND ART

An organic electroluminescence device (hereinafter, occasionally referred to as “organic EL device”) has found its application in a full-color display for mobile phones, televisions and the like. When a voltage is applied to an organic EL device, holes and electrons are injected from an anode and a cathode, respectively, into an emitting layer. The injected holes and electrons are recombined in the emitting layer to form excitons. Specifically, according to the electron spin statistics theory, singlet excitons and triplet excitons are generated at a ratio of 25%:75%.

For instance, in Patent Literatures 1 to 4 and 6, various studies have been made for compounds to be used for the organic EL device in order to enhance the performance of the organic EL device. In addition, Patent Literature 5 describes a phenomenon where singlet excitons are generated by collision and fusion of two triplet excitons (hereinafter, sometimes referred to as a Triplet-Triplet Fusion (TTF) phenomenon) in order to enhance the performance of the organic EL device.

The performance of the organic EL device is evaluable in terms of, for instance, luminance, emission wavelength, chromaticity, luminous efficiency, drive voltage, and lifetime.

### CITATION LIST

### Patent Literature(s)

Patent Literature 1: JP 2013-157552 A
Patent Literature 2: JP 2009-016478 A
Patent Literature 3: International Publication No. WO2007/138906
Patent Literature 4: US Patent Application Publication No. 2019/280209
Patent Literature 5: International Publication No. WO2010/134350
Patent Literature 6: JP 2007-294261 A

### SUMMARY OF THE INVENTION

### Problems to be Solved by the Invention

An object of the invention is to provide an organic electroluminescence device with enhanced performance. Another object of the invention is to provide an organic electroluminescence device with improved luminous efficiency and an electronic device including the organic electroluminescence device.

### Means for Solving the Problems

According to an aspect of the invention, there is provided an organic electroluminescence device including a first emitting layer and a second emitting layer, in which the first emitting layer includes a first host material, the second emitting layer includes a second host material, the first host material and the second host material are mutually different, the first emitting layer at least includes a compound that emits fluorescence having a main peak wavelength of 500 nm or less, the second emitting layer at least includes a compound that emits fluorescence having a main peak wavelength of 500 nm or less, the compound included in the first emitting layer and that emits fluorescence having a main peak wavelength of 500 nm or less is the same as or different from the compound included in the second emitting layer and that emits fluorescence having a main peak wavelength of 500 nm or less, and a triplet energy T₁(H1) of the first host material and a triplet energy T₁(H2) of the second host material satisfy a relationship of a numerical formula (Numerical Formula 1) below,

$${T}_{1} ( H 1 ) > {T}_{1} ( H 2 ) . \qquad ( Numerical Formula 1 )$$

According to another aspect of the invention, an electronic device including the organic electroluminescence device according to the above aspect of the invention is provided.

According to the above aspect of the invention, an organic electroluminescence device with enhanced performance can be provided. According to the above aspect of the invention, an organic electroluminescence device with improved luminous efficiency can be provided. According to the above aspect of the invention, an electronic device including the organic electroluminescence device can be provided.

### BRIEF DESCRIPTION OF DRAWING(S)

The FIGURE schematically shows an exemplary arrangement of an organic electroluminescence device according to an exemplary embodiment of the invention.

### DESCRIPTION OF EMBODIMENT(S)

### Definitions

Herein, a hydrogen atom includes isotope having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Herein, the ring carbon atoms refer to the number of carbon atoms among atoms forming a ring of a compound (e.g., a monocyclic compound, fused-ring compound, crosslinking compound, carbon ring compound, and heterocyclic compound) in which the atoms are bonded with each other to form the ring. When the ring is substituted by a substituent(s), carbon atom(s) contained in the substituent(s) is not counted in the ring carbon atoms. Unless otherwise specified, the same applies to the “ring carbon atoms” described later. For instance, a benzene ring has 6 ring carbon atoms, a naphthalene ring has 10 ring carbon atoms, a pyridine ring has 5 ring carbon atoms, and a furan ring has 4 ring carbon atoms. Further, for instance, 9,9-diphenylfluorenyl group has 13 ring carbon atoms and 9,9′-spirobifluorenyl group has 25 ring carbon atoms.

When a benzene ring is substituted by a substituent in a form of, for instance, an alkyl group, the number of carbon atoms of the alkyl group is not counted in the number of the ring carbon atoms of the benzene ring. Accordingly, the benzene ring substituted by an alkyl group has 6 ring carbon atoms. When a naphthalene ring is substituted by a substituent in a form of, for instance, an alkyl group, the number of carbon atoms of the alkyl group is not counted in the number of the ring carbon atoms of the naphthalene ring. Accordingly, the naphthalene ring substituted by an alkyl group has 10 ring carbon atoms.

Herein, the ring atoms refer to the number of atoms forming a ring of a compound (e.g., a monocyclic compound, fused-ring compound, cross-linking compound, carbon ring compound, and heterocyclic compound) in which the atoms are bonded to each other to form the ring (e.g., monocyclic ring, fused ring, and ring assembly). Atom(s) not forming the ring (e.g., hydrogen atom(s) for saturating the valence of the atom which forms the ring) and atom(s) in a substituent by which the ring is substituted are not counted as the ring atoms. Unless otherwise specified, the same applies to the “ring atoms” described later. For instance, a pyridine ring has 6 ring atoms, a quinazoline ring has 10 ring atoms, and a furan ring has 5 ring atoms. For instance, the number of hydrogen atom(s) bonded to a pyridine ring or the number of atoms forming a substituent are not counted as the pyridine ring atoms. Accordingly, a pyridine ring bonded to a hydrogen atom(s) or a substituent(s) has 6 ring atoms. For instance, the hydrogen atom(s) bonded to carbon atom(s) of a quinazoline ring or the atoms forming a substituent are not counted as the quinazoline ring atoms. Accordingly, a quinazoline ring bonded to hydrogen atom(s) or a substituent(s) has 10 ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group. Herein, “YY” is larger than “XX,” “XX” representing an integer of 1 or more and “YY” representing an integer of 2 or more.

Herein, “XX to YY atoms” in the description of “substituted or unsubstituted ZZ group having XX to YY atoms” represent atoms of an unsubstituted ZZ group and do not include atoms of a substituent(s) of the substituted ZZ group. Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s). The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent. Similarly, the term “substituted” used in a “BB group substituted by AA group” means that at least one hydrogen atom in the BB group is substituted with the AA group.

### Substituents Mentioned Herein

Substituents mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G1B) below. (Herein, an unsubstituted aryl group refers to an “unsubstituted aryl group” in a “substituted or unsubstituted aryl group,” and a substituted aryl group refers to a “substituted aryl group” in a “substituted or unsubstituted aryl group.”) A simply termed “aryl group” herein includes both of an “unsubstituted aryl group” and a “substituted aryl group.”

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by further substituting a hydrogen atom bonded to a carbon atom of a skeleton of a “substituted aryl group” in the specific example group G1B below, and a group derived by further substituting a hydrogen atom of a substituent of the “substituted aryl group” in the specific example group G1B below.

### Unsubstituted Aryl Group (Specific Example Group G1A):

a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, a phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, perylenyl group, and a monovalent aryl group derived by removing one hydrogen atom from cyclic structures represented by formulae (TEMP-1) to (TEMP-15) below.

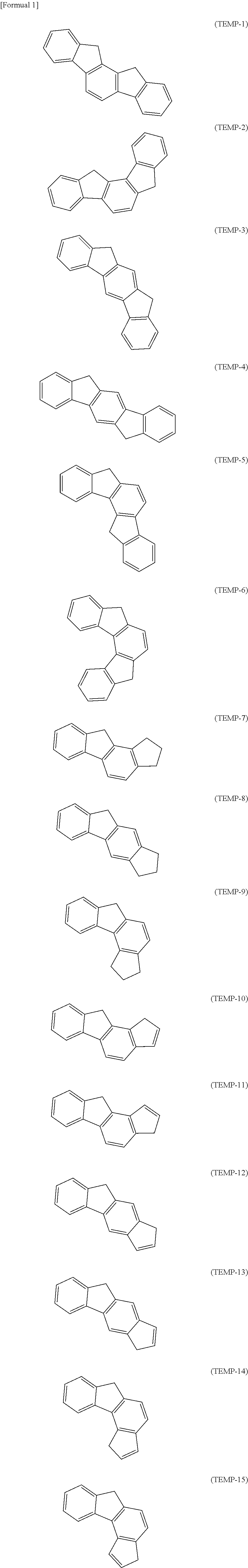

### Substituted Aryl Group (Specific Example Group G1B):

o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethylphenyl group, 9,9-dimethylfluorenyl group, 9,9-diphenylfluorenyl group, 9,9-bis(4-methylphenyl)fluorenyl group, 9,9-bis(4-isopropylphenyl)fluorenyl group, 9,9-bis(4-t-butylphenyl)fluorenyl group, cyanophenyl group, triphenylsilylphenyl group, trimethylsilylphenyl group, phenylnaphthyl group, naphthylphenyl group, and a group derived by substituting at least one hydrogen atom of a monovalent group derived from one of the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one hetero atom in the ring atoms. Specific examples of the hetero atom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B). Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”) A simply termed “heterocyclic group” herein includes both of an “unsubstituted heterocyclic group” and a “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by further substituting a hydrogen atom bonded to a ring atom of a skeleton of a “substituted heterocyclic group” in the specific example group G2B below, and a group derived by further substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

### Unsubstituted Heterocyclic Groups Including Nitrogen Atom (Specific Example Group G2A1):

pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, pyridyl group, pyridazynyl group, pyrimidinyl group, pyrazinyl group, triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

### Unsubstituted Heterocyclic Groups Including Oxygen Atom (Specific Example Group G2A2):

furyl group, oxazolyl group, isoxazolyl group, oxadiazolyl group, xanthenyl group, benzofuranyl group, isobenzofuranyl group, a dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

### Unsubstituted Heterocyclic Groups Including Sulfur Atom (Specific Example Group G2A3):

thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).
   Monovalent Heterocyclic Groups Derived by Removing One Hydrogen Atom from Cyclic Structures Represented by Formulae (TEMP-16) to (TEMP-33) (Specific Example Group G2A4):

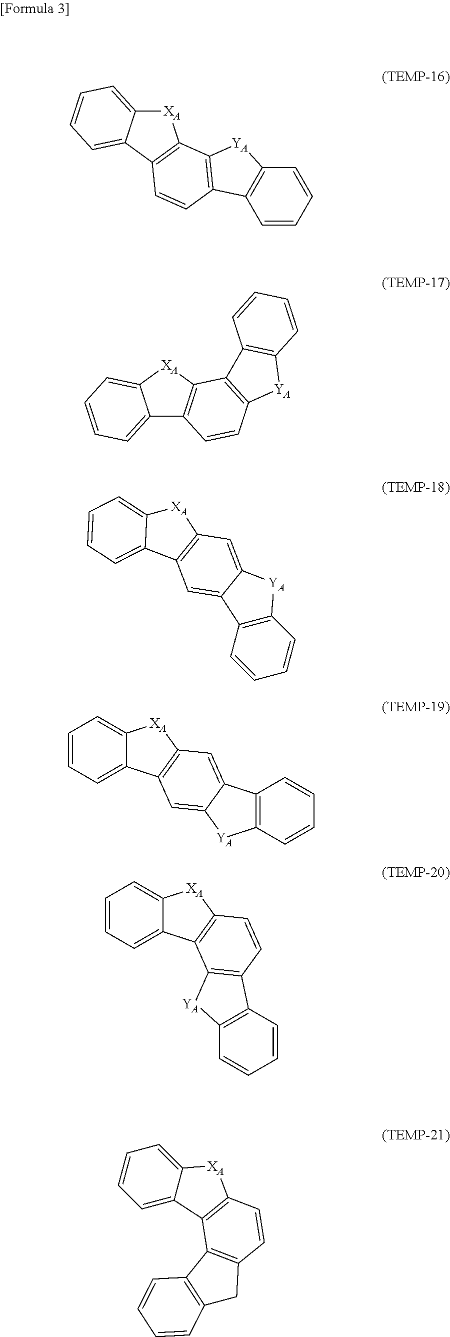

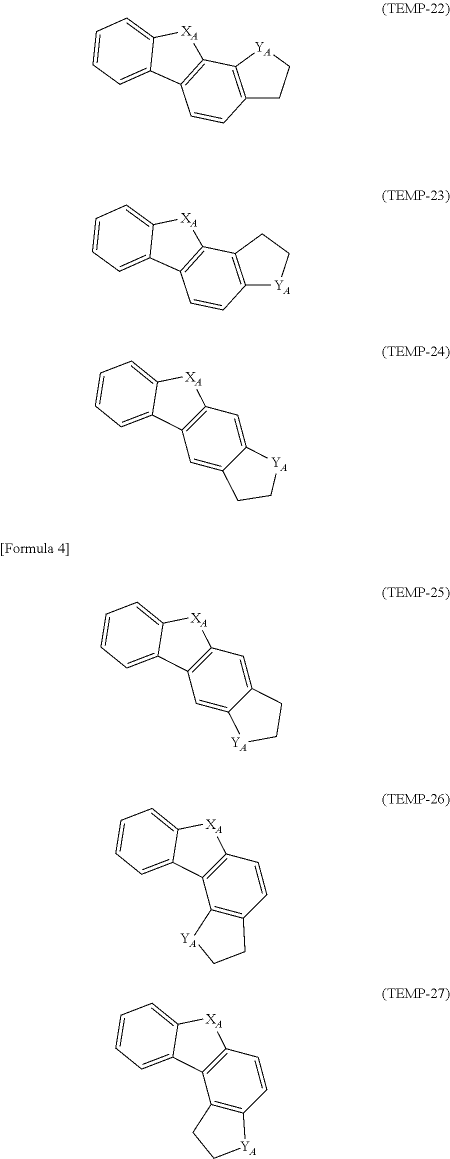

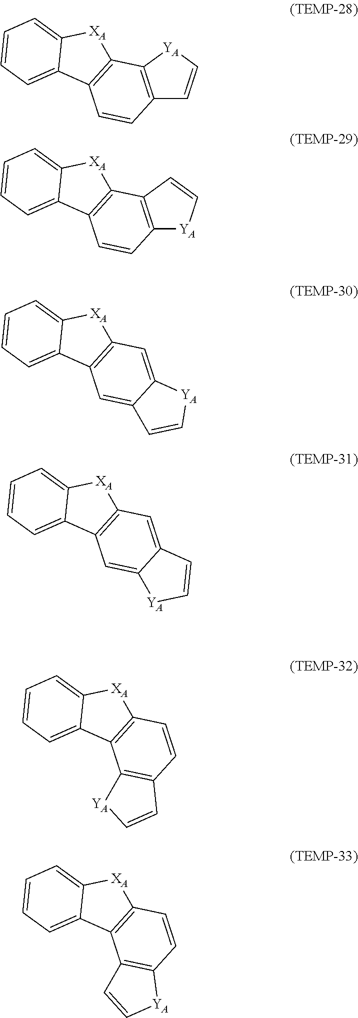

In the formulae (TEMP-16) to (TEMP-33), X_{A} and Y_{A} are each independently an oxygen atom, a sulfur atom, NH, or CH₂, with a proviso that at least one of X_{A} or Y_{A} is an oxygen atom, a sulfur atom, or NH.

When at least one of X_{A} or Y_{A} in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH or CH₂.

### Substituted Heterocyclic Groups Including Nitrogen Atom (Specific Example Group G2B1):

(9-phenyl)carbazolyl group, (9-biphenylyl)carbazolyl group, (9-phenyl)phenylcarbazolyl group, (9-naphthyl)carbazolyl group, diphenylcarbazole-9-yl group, phenylcarbazole-9-yl group, methylbenzimidazolyl group, ethylbenzimidazolyl group, phenyltriazinyl group, biphenylyltriazinyl group, diphenyltriazinyl group, biphenylquinazolinyl group, and biphenylquinazolinyl group.

### Substituted Heterocyclic Groups Including Oxygen Atom (Specific Example Group G2B2):

phenyldibenzofuranyl group, methyldibenzofuranyl group, t-butyldibenzofuranyl group, and monovalent residue of spiro[9H-xanthene-9,9′-[9H]fluorene].

### Substituted Heterocyclic Groups Including Sulfur Atom (Specific Example Group G2B3):

phenyldibenzothiophenyl group, methyldibenzothiophenyl group, t-butyldibenzothiophenyl group, and monovalent residue of spiro[9H-thioxanthene-9,9′-[9H]fluorene].
   Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of XA or YA in a form of NH, and a hydrogen atom of one of XA and YA in a form of a methylene group (CH2).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B) below. (Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.”) A simply termed “alkyl group” herein includes both of an “unsubstituted alkyl group” and a “substituted alkyl group.”

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by further substituting a hydrogen atom of a skeleton of the “substituted alkyl group” in the specific example group G3B, and a group derived by further substituting a hydrogen atom of a substituent of the “substituted alkyl group” in the specific example group G3B.

### Unsubstituted Alkyl Group (Specific Example Group G3A):

methyl group, ethyl group, n-propyl group, isopropyl group, n-butyl group, isobutyl group, s-butyl group, and t-butyl group.

### Substituted Alkyl Group (Specific Example Group G3B):

heptafluoropropyl group (including isomer thereof), pentafluoroethyl group, 2,2,2-trifluoroethyl group, and trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B). (Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.”) A simply termed “alkenyl group” herein includes both of an “unsubstituted alkenyl group” and a “substituted alkenyl group.”

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by further substituting a hydrogen atom of a skeleton of the “substituted alkenyl group” in the specific example group G4B with a substituent, and a group derived by further substituting a hydrogen atom of a substituent of the “substituted alkenyl group” in the specific example group G4B with a substituent.

### Unsubstituted Alkenyl Group (Specific Example Group G4A):

vinyl group, allyl group, 1-butenyl group, 2-butenyl group, and 3-butenyl group.

### Substituted Alkenyl Group (Specific Example Group G4B):

1,3-butanedienyl group, 1-methylvinyl group, 1-methylallyl group, 1,1-dimethylallyl group, 2-methylallyl group, and 1,2-dimethylallyl group.

### Substituted or Unsubstituted Alkynyl Group

Specific examples (specific example group G5) of the “substituted or unsubstituted alkynyl group” mentioned herein include unsubstituted alkynyl groups (specific example group G5A) below. (Herein, an unsubstituted alkynyl group refers to an “unsubstituted alkynyl group” in a “substituted or unsubstituted alkynyl group.”) A simply termed “alkynyl group” herein includes both of an “unsubstituted alkynyl group” and a “substituted alkynyl group.”

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

Unsubstituted Alkynyl Group (Specific Example Group G5A): ethynyl group.

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B). (Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.”) A simply termed “cycloalkyl group” herein includes both of an “unsubstituted cycloalkyl group” and a “substituted cycloalkyl group.”

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent.

### Unsubstituted Cycloalkyl Group (Specific Example Group G6A):

cyclopropyl group, cyclobutyl group, cyclopentyl group, cyclohexyl group, 1-adamantyl group, 2-adamantyl group, 1-norbornyl group, and 2-norbornyl group.

### Substituted Cycloalkyl Group (Specific Example Group G6B):

4-methylcyclohexyl group.
   Group Represented by —Si(R₉₀₁)(R₉₀₂)(R₉₀₃)

Specific examples (specific example group G7) of the group represented herein by —Si(R₉₀₁)(R₉₀₂)(R₉₀₃) include:
—Si(G1)(G1)(G1); —Si(G1)(G2)(G2); —Si(G1)(G1)(G2); —Si(G2)(G2)(G2); —Si(G3)(G3)(G3); and —Si(G6)(G6)(G6).
   Here: G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —Si(G1)(G1)(G1) are mutually the same or different;
a plurality of G2 in —Si(G1)(G2)(G2) are mutually the same or different;
a plurality of G1 in —Si(G1)(G1)(G2) are mutually the same or different;
a plurality of G2 in —Si(G2)(G2)(G2) are mutually the same or different;
a plurality of G3 in —Si(G3)(G3)(G3) are mutually the same or different; and
a plurality of G6 in —Si(G6)(G6)(G6) are mutually the same or different.

### Group Represented by —O—(R₉₀₄)

Specific examples (specific example group G8) of a group represented by —O—(R₉₀₄) herein include: —O(G1); —O(G2); —O(G3); and —O(G6).

Here:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.

### Group Represented by —S—(R₉₀₅)

Specific examples (specific example group G9) of a group represented herein by —S—(R₉₀₅) include: —S(G1); —S(G2); —S(G3); and —S(G6).

Here:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.
   Group Represented by —N(R₉₀₆)(R₉₀₇)

Specific examples (specific example group G10) of a group represented herein by —N(R₉₀₆)(R₉₀₇) include: —N(G1)(G1); —N(G2)(G2); —N(G1)(G2); —N(G3)(G3); and —N(G6)(G6).

Here:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —N(G1)(G1) are mutually the same or different;
a plurality of G2 in —N(G2)(G2) are mutually the same or different;
a plurality of G3 in —N(G3)(G3) are mutually the same or different; and
a plurality of G6 in —N(G6)(G6) are mutually the same or different.

### Halogen Atom

Specific examples (specific example group G11) of “halogen atom” mentioned herein include a fluorine atom, chlorine atom, bromine atom, and iodine atom.

### Substituted or Unsubstituted Fluoroalkyl Group

The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms. An “unsubstituted fluoroalkyl group” has, unless otherwise specified herein, 1 to 50, preferably 1 to 30, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein include a group derived by further substituting at least one hydrogen atom bonded to a carbon atom of an alkyl chain of a “substituted fluoroalkyl group” with a substituent, and a group derived by further substituting at least one hydrogen atom of a substituent of the “substituted fluoroalkyl group” with a substituent. Specific examples of the “substituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms. An “unsubstituted haloalkyl group” has, unless otherwise specified herein, 1 to 50, preferably 1 to 30, more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein include a group derived by further substituting at least one hydrogen atom bonded to a carbon atom of an alkyl chain of a “substituted haloalkyl group” with a substituent, and a group derived by further substituting at least one hydrogen atom of a substituent of the “substituted haloalkyl group” with a substituent. Specific examples of the “unsubstituted haloalkyl group” include a group derived by substituting at least one hydrogen atom of the “alkyl group” (specific example group G3) with a halogen atom. The haloalkyl group is sometimes referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50, preferably 1 to 30, more preferably 1 to 18 carbon atoms.

### Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50, preferably 6 to 30, more preferably 6 to 18 ring carbon atoms.

### Substituted or Unsubstituted Trialkylsilyl Group

Specific examples of a “trialkylsilyl group” mentioned herein include a group represented by —Si(G3)(G3)(G3), G3 being the “substituted or unsubstituted alkyl group” in the specific example group G3. The plurality of G3 in —Si(G3)(G3)(G3) are mutually the same or different. Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by (G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl)carbazolyl group ((9-phenyl)carbazole-1-yl group, (9-phenyl)carbazole-2-yl group, (9-phenyl)carbazole-3-yl group, or (9-phenyl)carbazole-4-yl group), (9-biphenylyl)carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

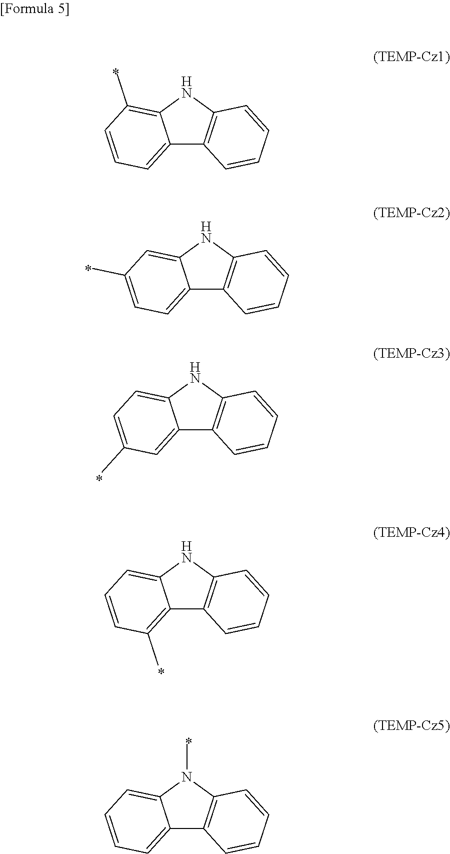

The (9-phenyl)carbazolyl group mentioned herein is, unless otherwise specified herein, specifically a group represented by one of formulae below.

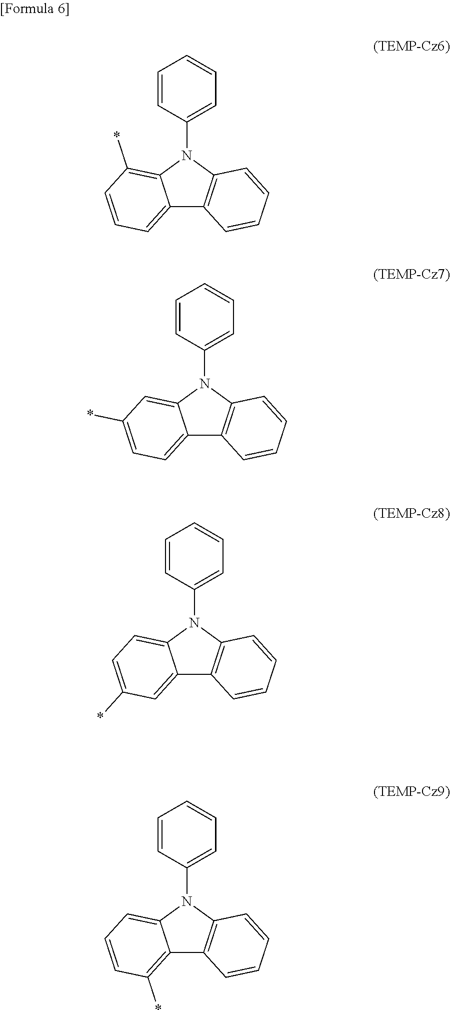 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the formulae (TEMP-Cz1) to (TEMP-Cz9), * represents a bonding position.

The dibenzofuranyl group and dibenzothiophenyl group mentioned herein are, unless otherwise specified herein, each specifically represented by one of formulae below.

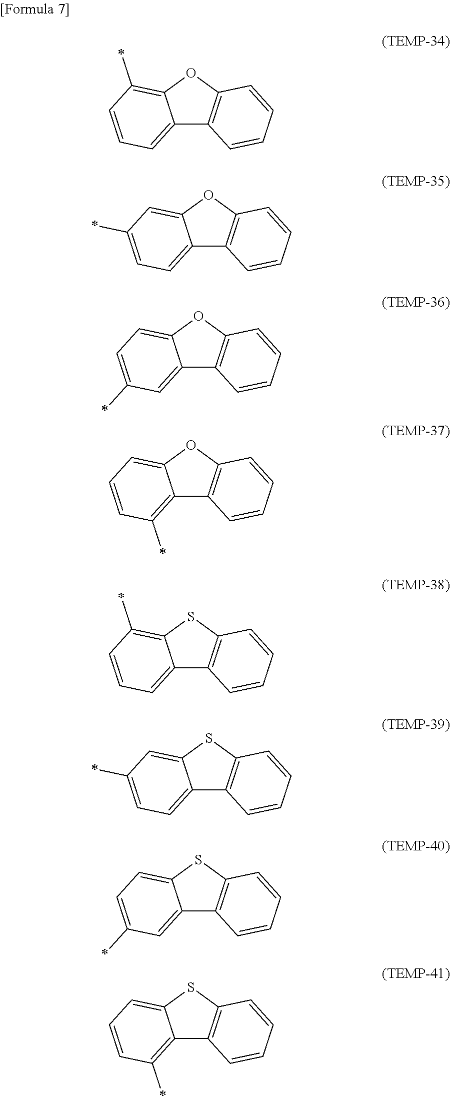

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.” Specific examples of the “substituted or unsubstituted arylene group” (specific example group G12) include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocycle of the “substituted or unsubstituted heterocyclic group.” Specific examples of the “substituted or unsubstituted divalent heterocyclic group” (specific example group G13) include a divalent group derived by removing one hydrogen atom on a heterocyclic ring of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived by removing one hydrogen atom on an alkyl chain of the “substituted or unsubstituted alkyl group.” Specific examples of the “substituted or unsubstituted alkylene group” (specific example group G14) include a divalent group derived by removing one hydrogen atom on an alkyl chain of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

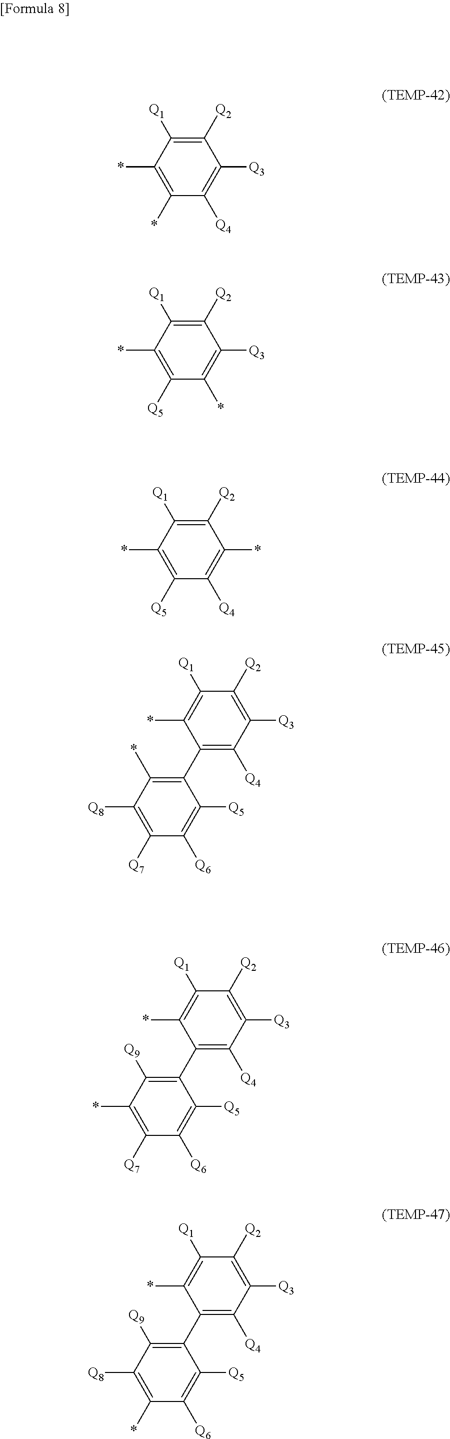

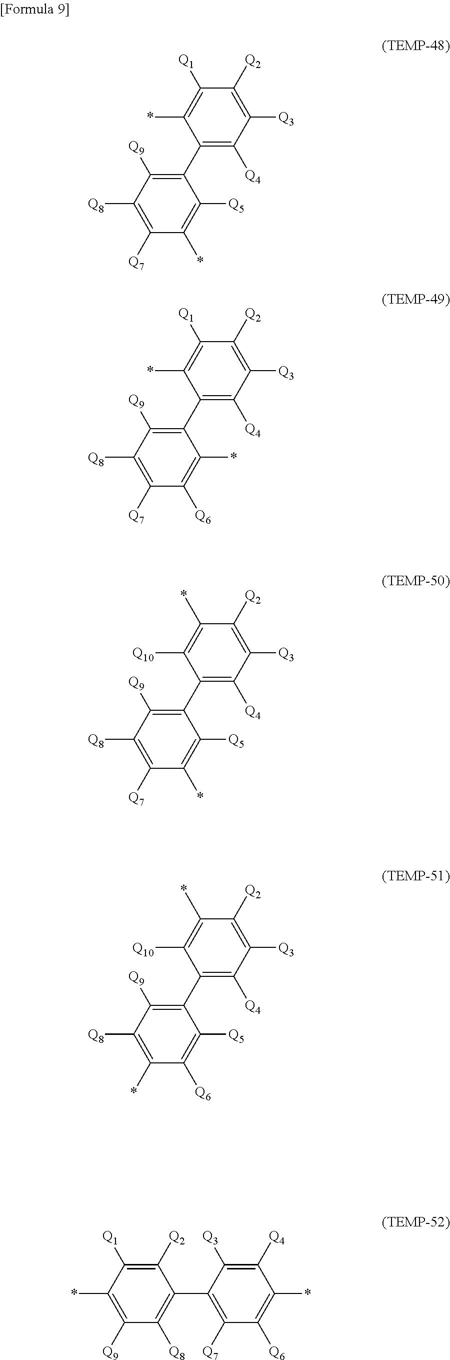

In the formulae (TEMP-42) to (TEMP-52), Q₁ to Q₁₀ each independently are a hydrogen atom or a substituent.

In the formulae (TEMP-42) to (TEMP-52), * represents a bonding position.

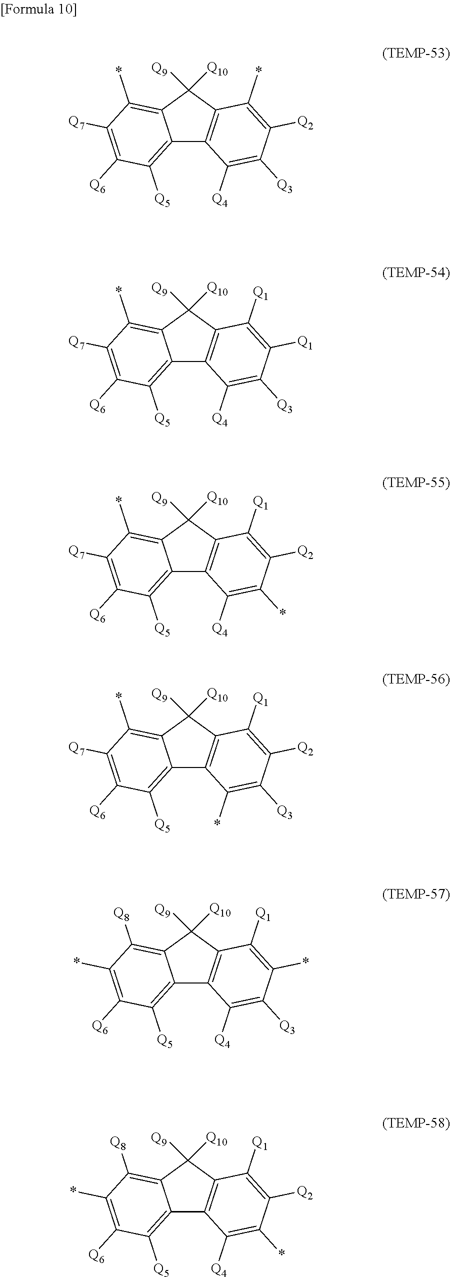

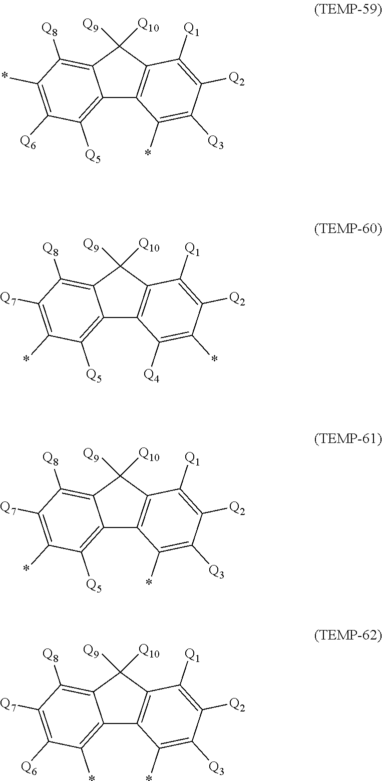

In the formulae (TEMP-53) to (TEMP-62), Q₁ to Q₁₀ each independently are a hydrogen atom or a substituent.

In the formulae, Q₉ and Q₁₀ may be mutually bonded through a single bond to form a ring.

In the formulae (TEMP-53) to (TEMP-62), * represents a bonding position.

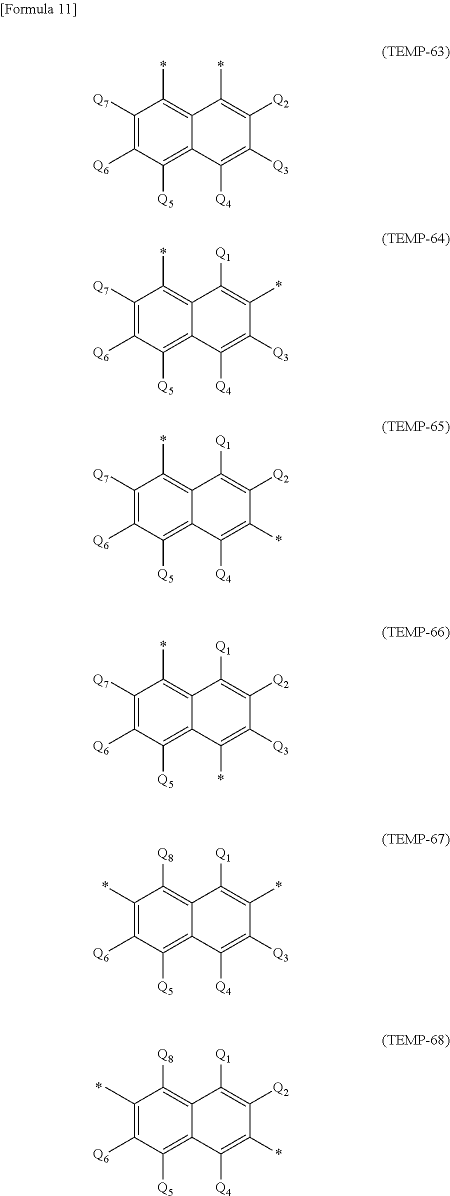

In the formulae (TEMP-63) to (TEMP-68), Q₁ to Q₈ each independently are a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

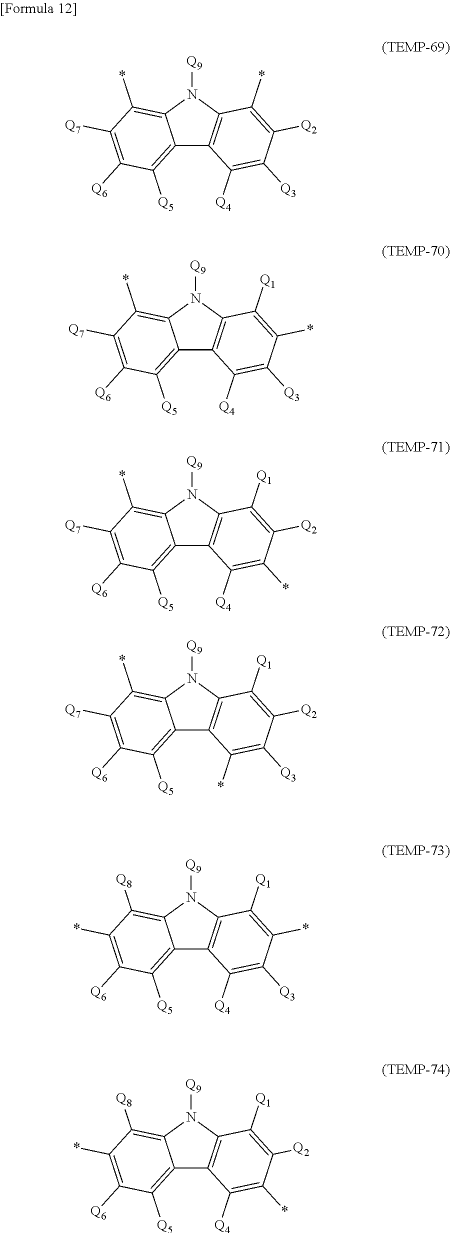

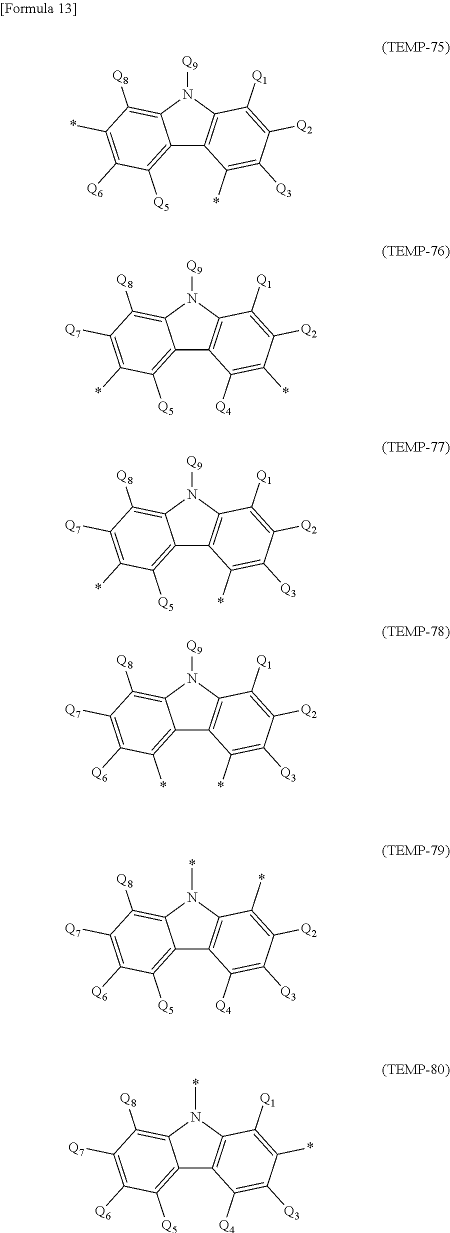

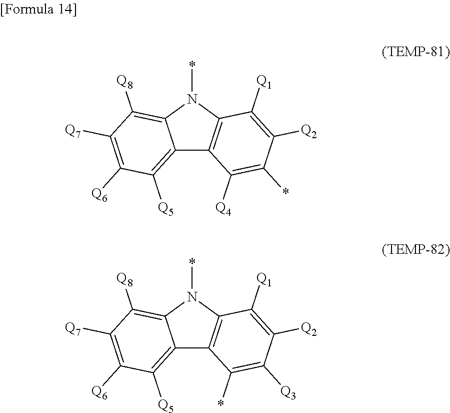

In the formulae (TEMP-69) to (TEMP-82), Q₁ to Q₉ each independently are a hydrogen atom or a substituent.

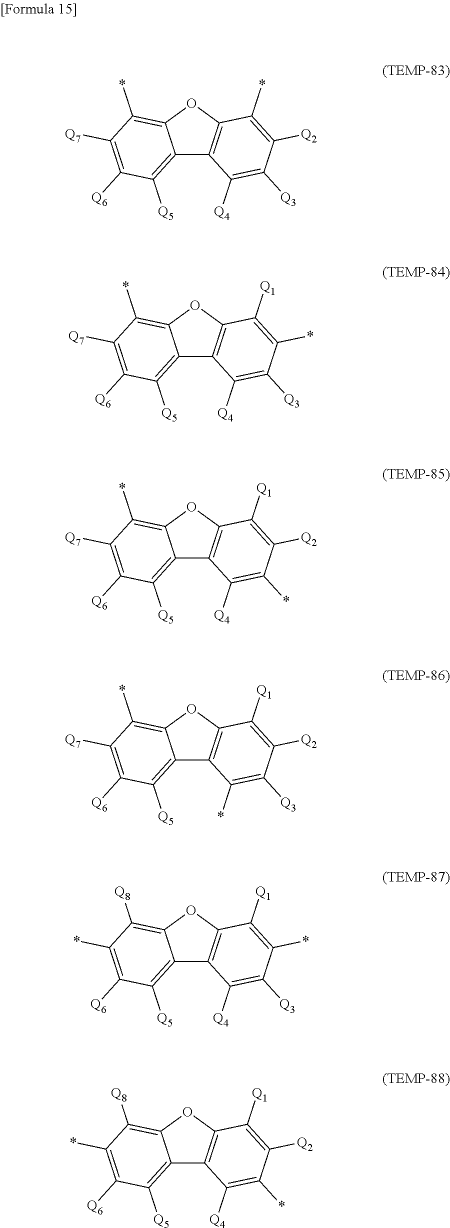

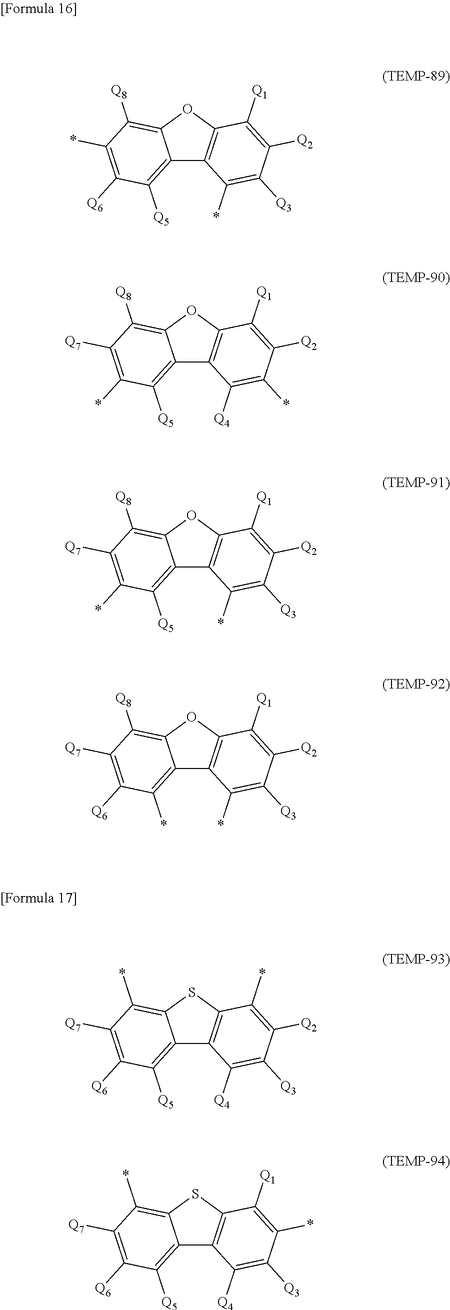

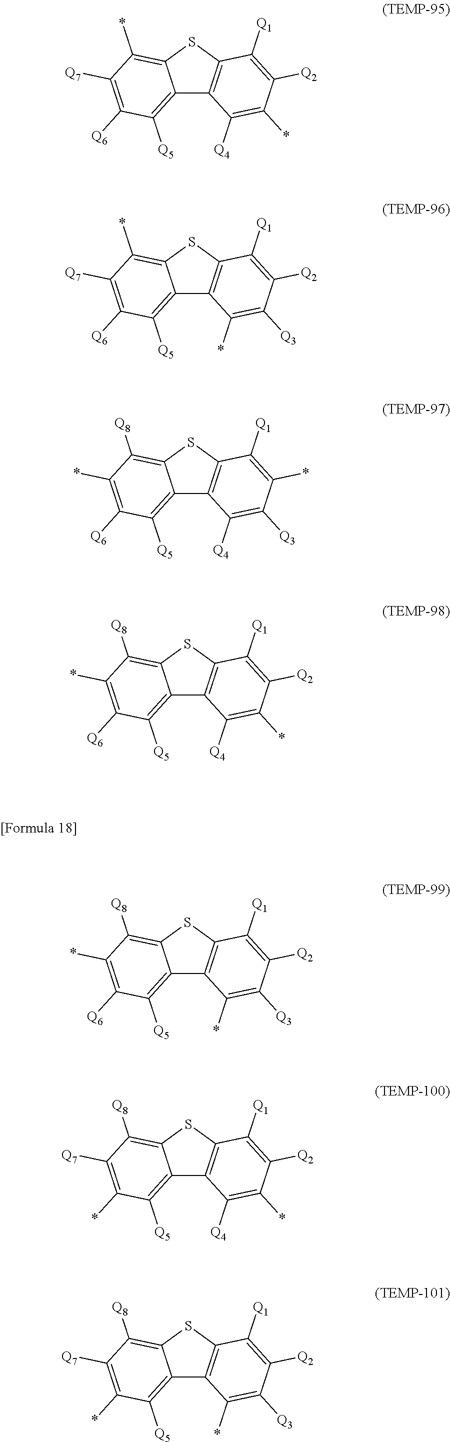

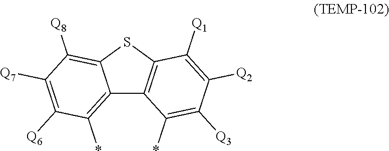

In the formulae (TEMP-83) to (TEMP-102), Q₁ to Q₈ each independently are a hydrogen atom or a substituent.

The substituent mentioned herein has been described above.

### Instance of “Bonded to Form Ring”

Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring, “at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (these instances will be sometimes collectively referred to as an instance of “bonded to form a ring” hereinafter) will be described below. An anthracene compound having a basic skeleton in a form of an anthracene ring and represented by a formula (TEMP-103) below will be used as an example for the description.

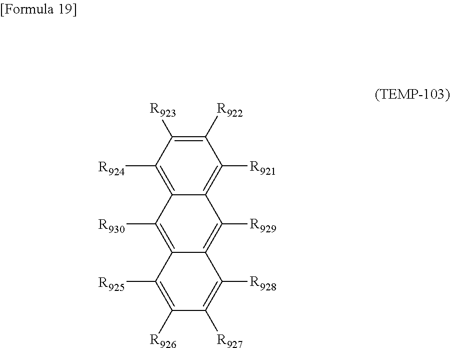

For instance, when “at least one combination of adjacent two or more of R₉₂₁ to R₉₃₀ are mutually bonded to form a ring,” the combination of adjacent ones of R₉₂₁ to R₉₃₀ (i.e. the combination at issue) is a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, or a combination of R₉₂₉ and R₉₂₁.

The term “at least one combination” means that two or more of the above combinations of adjacent two or more of R921 to R930 may simultaneously form rings. For instance, when R₉₂₁ and R₉₂₂ are mutually bonded to form a ring Q_{A} and R₉₂₅ and R₉₂₆ are simultaneously mutually bonded to form a ring Q_{B}, the anthracene compound represented by the formula (TEMP-103) is represented by a formula (TEMP-104) below.

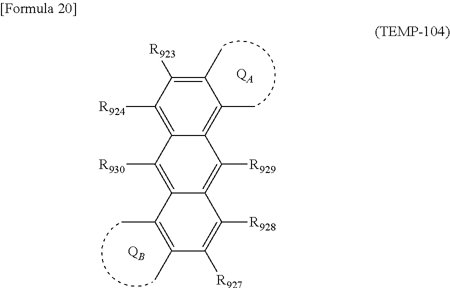

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring Q_{A} and R₉₂₂ and R₉₂₃ are mutually bonded to form a ring Q_{C}, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring Q_{A} and the ring Q_{C} share R₉₂₂.

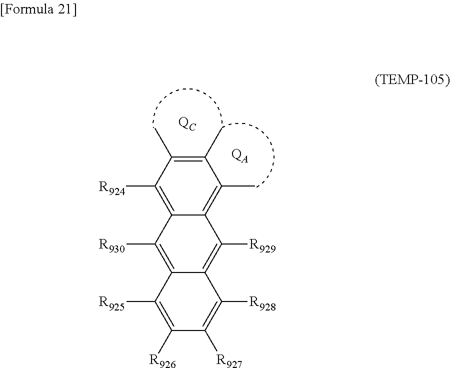

The formed “monocyclic ring” or “fused ring” may be, in terms of the formed ring in itself, a saturated ring or an unsaturated ring. When the “combination of adjacent two” form a “monocyclic ring” or a “fused ring,” the “monocyclic ring” or “fused ring” may be a saturated ring or an unsaturated ring. For instance, the ring Q_{A} and the ring Q_{B} formed in the formula (TEMP-104) are each independently a “monocyclic ring” or a “fused ring.” Further, the ring Q_{A} and the ring Q_{C} formed in the formula (TEMP-105) are each a “fused ring.” The ring Q_{A} and the ring Q_{C} in the formula (TEMP-105) are fused to form a fused ring. When the ring Q_{A} in the formula (TEMP-104) is a benzene ring, the ring Q_{A} is a monocyclic ring. When the ring Q_{A} in the formula (TEMP-104) is a naphthalene ring, the ring Q_{A} is a fused ring.

The “unsaturated ring” represents an aromatic hydrocarbon ring or an aromatic heterocycle. The “saturated ring” represents an aliphatic hydrocarbon ring or a non-aromatic heterocycle.

Specific examples of the aromatic hydrocarbon ring include a ring formed by terminating a bond of a group in the specific example of the specific example group G1 with a hydrogen atom.

Specific examples of the aromatic heterocycle include a ring formed by terminating a bond of an aromatic heterocyclic group in the specific example of the specific example group G2 with a hydrogen atom.

Specific examples of the aliphatic hydrocarbon ring include a ring formed by terminating a bond of a group in the specific example of the specific example group G6 with a hydrogen atom.

The phrase “to form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded to R₉₂₁, a carbon atom of the anthracene skeleton bonded to R₉₂₂, and one or more optional atoms. Specifically, when the ring Q_{A} is a monocyclic unsaturated ring formed by R₉₂₁ and R₉₂₂, the ring formed by a carbon atom of the anthracene skeleton bonded to R₉₂₁, a carbon atom of the anthracene skeleton bonded to R₉₂₂, and four carbon atoms is a benzene ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes an optional element other than carbon atom, the resultant ring is a heterocycle.

The number of “one or more optional atoms” forming the monocyclic ring or fused ring is, unless otherwise specified herein, preferably in a range from 2 to 15, more preferably in a range from 3 to 12, further preferably in a range from 3 to 5.

Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

When the “saturated ring” or the “unsaturated ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance of “bonded to form a ring”).

### Substituent for Substituted or Unsubstituted Group

In an exemplary embodiment herein, a substituent for the substituted or unsubstituted group (sometimes referred to as an “optional substituent” hereinafter) is, for instance, a group selected from the group consisting of an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms; R₉₀₁ to R₉₀₇ each independently are a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different; and
when two or more R₉₀₇ are present, the two or more R₉₀₇ are mutually the same or different.

In an exemplary embodiment, a substituent for the substituted or unsubstituted group is selected from the group consisting of an alkyl group having 1 to 50 carbon atoms, an aryl group having 6 to 50 ring carbon atoms, and a heterocyclic group having 5 to 50 ring atoms.

In an exemplary embodiment, a substituent for the substituted or unsubstituted group is selected from the group consisting of an alkyl group having 1 to 18 carbon atoms, an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted unsaturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

Herein, numerical ranges represented by “AA to BB” represent a range whose lower limit is the value (AA) recited before “to” and whose upper limit is the value (BB) recited after “to.”

### First Exemplary Embodiment

### Organic Electroluminescence Device

An organic electroluminescence device according to the exemplary embodiment includes a first emitting layer and a second emitting layer, in which the first emitting layer contains a first host material, the second emitting layer contains a second host material, the first host material and the second host material are mutually different, the first emitting layer at least contains a compound that emits fluorescence having a main peak wavelength of 500 nm or less, the second emitting layer at least contains a compound that emits fluorescence having a main peak wavelength of 500 nm or less, the compound that emits fluorescence having a main peak wavelength of 500 nm or less and contained in the first emitting layer is the same as or different from the compound that emits fluorescence having a main peak wavelength of 500 nm or less and contained in the second emitting layer, and a triplet energy T₁(H1) of the first host material and a triplet energy T₁(H2) of the second host material satisfy a numerical formula (Numerical Formula 1).

$${T}_{1} ( H 1 ) > {T}_{1} ( H 2 ) \qquad ( Numerical Formula 1 )$$

According to the exemplary embodiment, an organic electroluminescence device with improved luminous efficiency can be provided.

Conventionally, Triplet-Triplet-Annihilation (sometimes referred to as TTA) is known as a technique for enhancing the luminous efficiency of the organic electroluminescence device. TTA is a mechanism in which triplet excitons collide with one another to generate singlet excitons. It should be noted that the TTA mechanism is also occasionally referred to as a TTF mechanism as described in Patent Literature 4.

The TTF phenomenon will be described. Holes injected from an anode and electrons injected from a cathode are recombined in an emitting layer to generate excitons. As for the spin state, as is conventionally known, singlet excitons account for 25% and triplet excitons account for 75%. In a conventionally known fluorescent device, light is emitted when singlet excitons of 25% are relaxed to the ground state. The remaining triplet excitons of 75% are returned to the ground state without emitting light through a thermal deactivation process. Accordingly, the theoretical limit value of the internal quantum efficiency of a conventional fluorescent device is believed to be 25%.

Meanwhile, the behavior of triplet excitons generated within an organic substance has been theoretically examined. According to S. M. Bachilo et al. (J. Phys. Chem. A, 104, 7711 (2000)), assuming that high-order excitons such as quintet excitons are quickly returned to triplet excitons, triplet excitons (hereinafter abbreviated as 3A*) collide with one another with an increase in the density thereof, whereby a reaction shown by the following formula occurs. In the formula, ¹A represents the ground state and ¹A* represents the lowest singlet excitons.

$${}^{3} {A}^{*} + {}^{3} {A}^{*} \to {( 4 / 9 )}^{1} A + {( 1 / 9 )}^{1} {A}^{*} + {( 13 / 9 )}^{3} {A}^{*}$$

In other words, 5³A*→4¹A+1A* is satisfied, and it is expected that, among triplet excitons initially generated, which account for 75%, one fifth thereof (i.e., 20%) is changed to singlet excitons. Accordingly, the amount of singlet excitons which contribute to emission is 40%, which is a value obtained by adding 15% (75%×(⅕)=15%) to 25%, which is the amount ratio of initially generated singlet excitons. At this time, a ratio of luminous intensity derived from TTF (TTF ratio) relative to the total luminous intensity is 15/40, i.e., 37.5%. Assuming that singlet excitons are generated by collision of initially generated triplet excitons accounting for 75% (i.e., one singlet exciton is generated from two triplet excitons), a significantly high internal quantum efficiency of 62.5% is obtained, which is a value obtained by adding 37.5% (75%×(½)=37.5%) to 25% (the amount ratio of initially generated singlet excitons). At this time, the TTF ratio is 37.5/62.5=60%.

In the organic electroluminescence device of the exemplary embodiment, it is considered that triplet excitons generated by recombination of holes and electrons in the first emitting layer and present on an interface between the first emitting layer and organic layer(s) in direct contact therewith are not likely to be quenched even under the presence of excessive carriers on the interface between the first emitting layer and the organic layer(s). For instance, the presence of a recombination region locally on an interface between the first emitting layer and a hole transporting layer or an electron blocking layer is considered to cause quenching by excessive electrons. Meanwhile, the presence of a recombination region locally on an interface between the first emitting layer and an electron transporting layer or a hole blocking layer is considered to cause quenching by excessive holes.

The organic EL device of the exemplary embodiment includes at least two emitting layers (i.e., the first emitting layer and the second emitting layer) satisfying a predetermined relationship, in which a triplet energy T₁(H1) of the first host material in the first emitting layer and a triplet energy T₁(H2) of the second host material in the second emitting layer satisfy the relationship of the above numerical formula (Numerical Formula 1).

By including the first emitting layer and the second emitting layer so as to satisfy the numerical formula (Numerical Formula 1), triplet excitons generated in the first emitting layer can transfer to the second emitting layer without being quenched by excessive carriers and be inhibited from back-transferring from the second emitting layer to the first emitting layer. Consequently, the second emitting layer exhibits the TTF mechanism to efficiently generate singlet excitons, thereby improving luminous efficiency.

Accordingly, the organic electroluminescence device includes, as different regions, the first emitting layer mainly generating triplet excitons and the second emitting layer mainly exhibiting the TTF mechanism using triplet excitons having transferred from the first emitting layer, and a difference in triplet energy is provided by using a compound having a smaller triplet energy than that of the first host material in the first emitting layer as the second host material in the second emitting layer, thereby improving the luminous efficiency.

In the organic EL device according to the exemplary embodiment, the triplet energy T₁(H1) of the first host material and the triplet energy T₁(H2) of the second host material preferably satisfy a relationship of a numerical formula (Numerical Formula 5) below.

$${T}_{1} ( H 1 ) - {T}_{1} ( H 2 ) > 0.03 eV \qquad ( Numerical Formula 5 )$$

Herein, the “host material” refers to, for instance, a material that accounts for “50 mass % or more of the layer.” Accordingly, for instance, the first emitting layer contains the first host material at 50 mass % or more with respect to a total mass of the first emitting layer. For instance, the second emitting layer contains the second host material at 50 mass % or more with respect to a total mass of the second emitting layer.

### Emission Wavelength of Organic EL Device

The organic electroluminescence device of the exemplary embodiment preferably emit light having a main peak wavelength of 500 nm or less when the device is driven.

The organic electroluminescence device of the exemplary embodiment more preferably emits light having a main peak wavelength in a range from 430 nm to 480 nm when the device is driven.

The main peak wavelength of the light emitted from the organic EL device when being driven is measured as follows. Voltage is applied on the organic EL devices such that a current density becomes 10 mA/cm², where spectral radiance spectrum is measured by a spectroradiometer CS-2000 (manufactured by Konica Minolta, Inc.). A peak wavelength of an emission spectrum, at which the luminous intensity of the resultant spectral radiance spectrum is at the maximum, is measured and defined as the main peak wavelength (unit: nm).

### First Emitting Layer

The first emitting layer contains the first host material. The first host material and the second host material contained in the second emitting layer are different compounds.

The first emitting layer at least contains a compound that emits fluorescence having a main peak wavelength of 500 nm or less. This “compound that emits fluorescence having a main peak wavelength of 500 nm or less” may be the first host material or a compound different from the first host material.

In the organic EL device of the exemplary embodiment, it is preferable that the first emitting layer further contains a first dopant material and the first dopant material is a fluorescent compound.

In the organic EL device of the exemplary embodiment, the first dopant material is preferably a compound not including an azine ring structure in a molecule.

In the organic EL device of the exemplary embodiment, the first dopant material is preferably not a boron-containing complex, and the first dopant material is more preferably not a complex.

In the organic EL device of the exemplary embodiment, the first emitting layer preferably does not contain a metal complex. In the organic EL device of the exemplary embodiment, the first emitting layer also preferably does not contain a boron-containing complex.

In the organic EL device of the exemplary embodiment, the first emitting layer preferably does not contain a phosphorescent material (dopant material).

The first emitting layer also preferably does not contain a heavy-metal complex and a phosphorescent rare earth metal complex. Examples of the heavy-metal complex herein include iridium complex, osmium complex, and platinum complex.

In the organic EL device of the exemplary embodiment, the first dopant material is preferably the compound that emits fluorescence having a main peak wavelength of 500 nm or less.

A measurement method of a main peak wavelength of the compound is as follows. A toluene solutions of each of measurement target compounds at a concentration of 5 μmol/L is prepared and put in a quartz cell. An emission spectrum (ordinate axis: emission intensity, abscissa axis: wavelength) of each sample is measured at a normal temperature (300K). The emission spectrum can be measured using a spectrophotometer (machine name: F-7000) manufactured by Hitachi High-Tech Science Corporation. It should be noted that the machine for measuring the emission spectrum is not limited to the machine used herein.

A peak wavelength of the emission spectrum exhibiting the maximum luminous intensity is defined as a main peak wavelength. It should be noted that the main peak wavelength is sometimes referred to as a fluorescence main peak wavelength (FL-peak) herein.

In an emission spectrum of the first dopant material, where a peak exhibiting a maximum luminous intensity is defined as a maximum peak and a height of the maximum peak is defined as 1, heights of other peaks appearing in the emission spectrum are preferably less than 0.6. It should be noted that the peaks in the emission spectrum are defined as local maximum values.

Moreover, in the emission spectrum of the first dopant material, the number of peaks is preferably less than three.

In the organic EL device of the exemplary embodiment, the first emitting layer preferably emits light having a main peak wavelength of 500 nm or less when the device is driven.

The main peak wavelength of light radiated from the organic EL device when being driven is measured by a method described in the section of later-described Examples.

In the organic EL device according to the exemplary embodiment, singlet energy S₁(H1) of the first host material and singlet energy S₁(D1) of the first dopant material preferably satisfy a relationship of the following numerical formula (Numerical Formula 2).

$${S}_{1} ( H 1 ) > {S}_{1} ( D 1 ) \qquad ( Numerical Formula 2 )$$

The singlet energy S₁ means an energy difference between the lowest singlet state and the ground state.

When the first host material and the first dopant material satisfy the relationship of the above numerical formula (Numerical Formula 2), singlet excitons generated on the first host material easily energy-transfer from the first host material to the first dopant material, thereby contributing to fluorescence of the first dopant material.

In the organic EL device according to the exemplary embodiment, the triplet energy T₁(H1) of the first host material and a triplet energy T₁(D1) of the first dopant material preferably satisfy a relationship of the following numerical formula (Numerical Formula 2A).

$${T}_{1} ( D 1 ) > {T}_{1} ( H 1 ) \qquad ( Numerical Formula 2 A )$$

When the first host material and the first dopant material satisfy the relationship of the numerical formula (Numerical Formula 2A), triplet excitons generated in the first emitting layer are transferred not onto the first dopant material having higher triplet energy but onto the first host material, thereby being easily transferred to the second emitting layer.

The organic EL device of the exemplary embodiment preferably satisfies a relationship of a numerical formula (Numerical Formula 2B) below.

$${T}_{1} ( D 1 ) > {T}_{1} ( H 1 ) > {T}_{1} ( H 2 ) \qquad ( Numerical Formula 2 B )$$

### Triplet Energy T₁

A method of measuring triplet energy T₁ is exemplified by a method below.

A measurement target compound is dissolved in EPA (diethylether:isopentane:ethanol=5:5:2 in volume ratio) so as to fall within a range from 10⁻⁵ mol/L to 10⁻⁴ mol/L, and the obtained solution is put in a quartz cell to provide a measurement sample. A phosphorescence spectrum (ordinate axis: phosphorescent luminous intensity, abscissa axis: wavelength) of the measurement sample is measured at a low temperature (77K). A tangent is drawn to the rise of the phosphorescence spectrum close to the short-wavelength region. An energy amount is calculated by a conversion equation (F1) below on a basis of a wavelength value λ_{edge} [nm] at an intersection of the tangent and the abscissa axis. The calculated energy amount is defined as triplet energy T₁.

$${T}_{1} [ eV ] = 1239.85 / {λ}_{edge} \qquad Conversion Equation ( F1 )$$

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength region of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted in the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 (manufactured by Hitachi High-Technologies Corporation) is usable. Any device for phosphorescence measurement is usable. A combination of a cooling unit, a low temperature container, an excitation light source and a light-receiving unit may be used for phosphorescence measurement.

### Singlet Energy S₁

A method of measuring a singlet energy S₁ with use of a solution (occasionally referred to as a solution method) is exemplified by a method below.

A toluene solution of a measurement target compound at a concentration ranging from 10⁻⁵ mol/L to 10⁻⁴ mol/L is prepared and put in a quartz cell. An absorption spectrum (ordinate axis: absorption intensity, abscissa axis: wavelength) of the thus-obtained sample is measured at a normal temperature (300K). A tangent is drawn to the fall of the absorption spectrum on the long-wavelength side, and a wavelength value λedge (nm) at an intersection of the tangent and the abscissa axis is assigned to a conversion equation (F2) below to calculate singlet energy.

$${S}_{1} [ eV ] = 1239.85 / {λ}_{edge} \qquad Conversion Equation ( F2 )$$

Any device for measuring absorption spectrum is usable. For instance, a spectrophotometer (U3310 manufactured by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The maximum absorbance of 0.2 or less is not counted as the above-mentioned local maximum absorbance closest to the long-wavelength region.

In the organic EL device of the exemplary embodiment, an electron mobility pH1 of the first host material and an electron mobility pH2 of the second host material preferably satisfy a relationship of a numerical formula (Numerical Formula 6) below.

$$μ H 2 > μ H 1 \qquad ( Numerical Formula 6 )$$

When the first host material and the second host material satisfy the relationship of the numerical formula (Numerical Formula 6), a recombination ability of holes and electrons in the first emitting layer is improved.

The electron mobility can be measured according to impedance spectroscopy.

A measurement target layer having a thickness in a range from 100 nm to 200 nm is held between the anode and the cathode, to which a small alternating voltage of 100 mV or less is applied while a bias DC voltage is applied. A value of an alternating current (absolute value and phase) which flows at this time is measured. This measurement is performed while changing a frequency of the alternating voltage, and complex impedance (Z) is calculated from the current value and the voltage value. A frequency dependency of the imaginary part (ImM) of the modulus M=iωZ (i: imaginary unit, ω: angular frequency) is obtained. The reciprocal number of a frequency ω at which the ImM becomes the maximum is defined as a response time of electrons carried in the measurement target layer. The electron mobility is calculated by the following equation.

$$Electron Mobility = {( Film Thickness of Measurement Target Layer )}^{2} / \\ ( Response Time \cdot Voltage )$$

In the organic EL device of the exemplary embodiment, the first dopant material is preferably contained at more than 1.1 mass % in the first emitting layer. Specifically, the first emitting layer preferably contains the first dopant material at more than 1.1 mass % with respect to a total mass of the first emitting layer, more preferably at more than 1.2 mass % with respect to the total mass of the first emitting layer, further preferably at more than 1.5 mass % with respect to the total mass of the first emitting layer.

The first emitting layer preferably contains the first dopant material at 10 mass % or less with respect to the total mass of the first emitting layer, more preferably at 7 mass % or less with respect to the total mass of the first emitting layer, further preferably at 5 mass % or less with respect to the total mass of the first emitting layer.

In the organic EL device of the exemplary embodiment, the first emitting layer preferably contains the first emitting compound as the first host material at 60 mass % or more with respect to the total mass of the first emitting layer, more preferably at 70 mass % or more with respect to the total mass of the first emitting layer, further preferably at 80 mass % or more with respect to the total mass of the first emitting layer, more further preferably at 90 mass % or more with respect to the total mass of the first emitting layer, still further more preferably at 95 mass % or more with respect to the total mass of the first emitting layer.

The first emitting layer preferably contains the first host material at 99 mass % or less with respect to the total mass of the first emitting layer.

In a case where the first emitting layer contains the first host material and the first dopant material, an upper limit of a total of the content ratios of the first host material and the first dopant material is 100 mass %.

It is not excluded that the first emitting layer of the exemplary embodiment further contains a material(s) other than the first host material and the first dopant material.

The first emitting layer may include a single type of the first host material or may include two or more types of the first host material. The first emitting layer may include a single type of the first dopant material or may include two or more types of the first dopant material.

A film thickness of the first emitting layer of the organic EL device in the exemplary embodiment is preferably 3 nm or more, more preferably 5 nm or more. The film thickness of the first emitting layer being 3 nm or more is a film thickness enough for causing recombination of holes and electrons in the first emitting layer.

The film thickness of the first emitting layer of the organic EL device in the exemplary embodiment is preferably 15 nm or less, more preferably 10 nm or less. The film thickness of the first emitting layer being 15 nm or less is a film thickness thin enough for transfer of triplet excitons to the second emitting layer.

The film thickness of the first emitting layer of the organic EL device in the exemplary embodiment is more preferably in a range from 3 nm to 15 nm.

### Second Emitting Layer

The second emitting layer contains the second host material. The second host material is a different compound from the first host material contained in the first emitting layer.

The second emitting layer at least contains a compound that emits fluorescence having a main peak wavelength of 500 nm or less. This “compound that emits fluorescence having a main peak wavelength of 500 nm or less” may be the second host material or a compound different from the second host material.

A measurement method of the main peak wavelength of the compound is as described above.

In the organic EL device of the exemplary embodiment, it is preferable that the second emitting layer further contains the second dopant material and the second dopant material is a fluorescent compound.

In the organic EL device of the exemplary embodiment, the second dopant material is preferably the compound that emits fluorescence having a main peak wavelength of 500 nm or less.

In the organic EL device of the exemplary embodiment, the second emitting layer preferably emits light having a main peak wavelength of 500 nm or less when the device is driven.

In the organic EL device of the exemplary embodiment, a full width at half maximum of the main peak of the second dopant material is preferably in a range from 1 nm to 20 nm.

In the organic EL device of the exemplary embodiment, a Stokes shift of the second dopant material preferably exceeds 7 nm.

When the Stokes shift of the second dopant material exceeds 7 nm, a reduction in the luminous efficiency due to self-absorption is likely to be inhibited.

The self-absorption is a phenomenon that emitted light is absorbed by the same compound to reduce luminous efficiency. The self-absorption is notably observed in a compound having a small Stokes shift (i.e., a large overlap between an absorption spectrum and a fluorescence spectrum). Accordingly, in order to reduce the self-absorption, it is preferable to use a compound having a large Stokes shift (i.e., a small overlap between the absorption spectrum and the fluorescence spectrum). The Stokes shift can be measured by a method described in Examples.

In the organic EL device of the exemplary embodiment, a triplet energy T₁(D2) of the second dopant material and the triplet energy T₁(H2) of the second host material preferably satisfy a relationship of a numerical formula (Numerical Formula 3) below.

$${T}_{1} ( D 2 ) > {T}_{1} ( H 2 ) \qquad ( Numerical Formula 3 )$$

In the organic EL device according to the exemplary embodiment, when the second dopant material and the second host material satisfy the relationship of the numerical formula (Numerical Formula 3), in transfer of triplet excitons generated in the first emitting layer to the second emitting layer, the triplet excitons energy-transfer not to the second dopant material having higher triplet energy but to molecules of the second host material. In addition, triplet excitons generated by recombination of holes and electrons on the second host material do not transfer to the second dopant material having higher triplet energy. Triplet excitons generated by recombination on molecules of the second dopant material quickly energy-transfer to molecules of the second host material.

Triplet excitons in the second host material do not transfer to the second dopant material but efficiently collide with one another on the second host material to generate singlet excitons by the TTF phenomenon.

In the organic EL device of the exemplary embodiment, a singlet energy S₁(H2) of the second host material and a singlet energy S₁(D2) of the second dopant material preferably satisfy a relationship of a numerical formula (Numerical Formula 4) below.

$${S}_{1} ( H 2 ) > {S}_{1} ( D 2 ) \qquad ( Numerical Formula 4 )$$

In the organic EL device according to the exemplary embodiment, when the second dopant material and the second host material satisfy the relationship of the numerical formula (Numerical formula 4), due to the singlet energy of the second dopant material being lower than the singlet energy of the second host material, singlet excitons generated by the TTF phenomenon energy-transfer from the second host material to the second dopant material, thereby contributing to fluorescence of the second dopant material.

In the organic EL device of the exemplary embodiment, it is preferable that the second dopant material is a compound not having an azine ring structure in a molecule.

In the organic EL device of the exemplary embodiment, the second dopant material is preferably not a boron-containing complex, and the second dopant material is more preferably not a complex.

In the organic EL device of the exemplary embodiment, it is preferable that the second emitting layer does not contain a metal complex. Further, in the organic EL device of the exemplary embodiment, it is also preferable that the second emitting layer does not contain a boron-containing complex.

In the organic EL device of the exemplary embodiment, it is preferable that the second emitting layer does not contain a phosphorescent material (dopant material).

Further, it is preferable that the second emitting layer does not contain a heavy metal complex and a phosphorescent rare earth metal complex. Examples of the heavy-metal complex herein include iridium complex, osmium complex, and platinum complex.

In the organic EL device of the exemplary embodiment, the second dopant material is preferably contained at more than 1.1 mass % in the second emitting layer. That is, the second emitting layer preferably contains the second dopant material at more than 1.1 mass % with respect to a total mass of the second emitting layer, more preferably at more than 1.2 mass % with respect to the total mass of the second emitting layer, further preferably at more than 1.5 mass % with respect to the total mass of the second emitting layer.

The second emitting layer preferably contains the second dopant material at 10 mass % or less with respect to the total mass of the second emitting layer, more preferably at 7 mass % or less with respect to the total mass of the second emitting layer, further preferably at 5 mass % or less with respect to the total mass of the second emitting layer.

The second emitting layer preferably contains a second compound as the second host material at 60 mass % or more with respect to the total mass of the second emitting layer, more preferably at 70 mass % or more with respect to the total mass of the second emitting layer, further preferably at 80 mass % or more with respect to the total mass of the second emitting layer, further more preferably at 90 mass % or more with respect to the total mass of the second emitting layer, still further preferably at 95 mass % or more with respect to the total mass of the second emitting layer.

The second emitting layer preferably contains the second host material at 99 mass % or less with respect to the total mass of the second emitting layer.

It should be noted that when the second emitting layer contains the second host material and the second dopant material, an upper limit of the total of the respective content ratios of the second host material and the second dopant material is 100 mass %.

It is not excluded that the second emitting layer according to the exemplary embodiment further contains a material(s) other than the second host material and the second dopant material.

The second emitting layer may include a single type of the second host material or may include two or more types of the second host material. The second emitting layer may include a single type of the second dopant material or may include two or more types of the second dopant material.

In the organic EL device according to the exemplary embodiment, the film thickness of the second emitting layer is preferably 5 nm or more, more preferably 15 nm or more. When the film thickness of the second emitting layer is 5 nm or more, it is easy to inhibit triplet excitons having transferred from the first emitting layer to the second emitting layer from returning to the first emitting layer. Further, when the film thickness of the second emitting layer is 5 nm or more, triplet excitons can be sufficiently separated from the recombination portion on the first emitting layer.

In the organic EL device according to the exemplary embodiment, the film thickness of the second emitting layer is preferably 20 nm or less. When the film thickness of the second emitting layer is 20 nm or less, the density of the triplet excitons in the second emitting layer is improved to cause the TTF phenomenon more easily.

In the organic EL device according to the exemplary embodiment, the film thickness of the second emitting layer is preferably in a range from 5 nm to 20 nm.

In the organic EL device of the exemplary embodiment, a triplet energy T₁(DX) of the compound contained in the first emitting layer and emitting fluorescence having a main peak wavelength of 500 nm or less or the compound contained in the second emitting layer and emitting fluorescence having a main peak wavelength of 500 nm or less, a triplet energy T₁(H1) of the first host material, and a triplet energy T₁(H2) of the second host material preferably satisfy a relationship of a numerical formula (Numerical Formula 10) below.

$$2.6 eV > {T}_{1} ( DX ) > {T}_{1} ( H 1 ) > {T}_{1} ( H 2 ) \qquad ( Numerical Formula 10 )$$

When the first emitting layer contains the first dopant material, the triplet energy T₁(D1) of the first dopant material preferably satisfies a relationship of a numerical formula (Numerical Formula 10A) below.

$$2.6 eV > {T}_{1} ( D 1 ) > {T}_{1} ( H 1 ) > {T}_{1} ( H 2 ) \qquad ( Numerical Formula 10 A )$$

When the second emitting layer contains the second dopant material, the triplet energy T₁(D2) of the second dopant material preferably satisfies a relationship of a numerical formula (Numerical Formula 10B) below.

$$2.6 eV > {T}_{1} ( D 2 ) > {T}_{1} ( H 1 ) > {T}_{1} ( H 2 ) \qquad ( Numerical Formula 10 B )$$

In the organic EL device of the exemplary embodiment, the triplet energy T₁(DX) of the compound contained in the first emitting layer and emitting fluorescence having a main peak wavelength of 500 nm or less or the compound contained in the second emitting layer and emitting fluorescence having a main peak wavelength of 500 nm or less, and the triplet energy T₁(H1) of the first host material preferably satisfy a relationship of a numerical formula (Numerical Formula 11) below.

$$0 eV < {T}_{1} ( DX ) - {T}_{1} ( H 1 ) < 0.6 eV \qquad ( Numerical Formula 11 )$$

When the first emitting layer contains the first dopant material, the triplet energy T₁(D1) of the first dopant material preferably satisfies a relationship of a numerical formula (Numerical Formula 11A) below.

$$0 eV < {T}_{1} ( D 1 ) - {T}_{1} ( H 1 ) < 0.6 eV \qquad ( Numerical Formula 11 A )$$

When the second emitting layer contains the second dopant material, the triplet energy T₁(D2) of the second dopant material preferably satisfies a relationship of a numerical formula (Numerical Formula 11B) below.

$$0 eV < {T}_{1} ( D 2 ) - {T}_{1} ( H 2 ) < 0.8 eV \qquad ( Numerical Formula 11 B )$$

In the organic EL device according to the present exemplary embodiment, the triplet energy T₁(H1) of the first host material preferably satisfies a relationship of a numerical formula (Numerical Formula 12) below.

$${T}_{1} ( H 1 ) > 2. eV \qquad ( Numerical Formula 12 )$$

In the organic EL device according to the present exemplary embodiment, the triplet energy T₁(H1) of the first host material also preferably satisfies a relationship of a numerical formula (Numerical Formula 12A) below, or also preferably satisfies a relationship of a numerical formula (Numerical Formula 12B) below.

$${T}_{1} ( H 1 ) > 2.1 eV . \qquad ( Numerical Formula 12 A )$$

$${T}_{1} ( H 1 ) > 2.15 eV \qquad ( Numerical Formula 12 B )$$

In the organic EL device according to the present exemplary embodiment, when the triplet energy T₁(H1) of the first host material satisfies the relationship of the numerical formula (Numerical Formula 12A) or the numerical formula (Numerical Formula 12B), triplet excitons generated in the first emitting layer are easily transferred to the second emitting layer, and also easily inhibited from back-transferring from the second emitting layer to the first emitting layer. Consequently, singlet excitons are efficiently generated, thereby improving luminous efficiency.

In the organic EL device according to the present exemplary embodiment, the triplet energy T₁(H1) of the first host material also preferably satisfies a relationship of a numerical formula (Numerical Formula 12C) below, or also preferably satisfies a relationship of a numerical formula (Numerical Formula 12D) below.

$$2.08 eV > {T}_{1} ( H 1 ) > 1.87 eV \qquad ( Numerical Formula 12 C )$$

$$2.05 eV > {T}_{1} ( H 1 ) > 1.9 eV \qquad ( Numerical Formula 12 D )$$

In the organic EL device according to the present exemplary embodiment, when the triplet energy T₁(H1) of the first host material satisfies the relationship of the numerical formula (Numerical Formula 12C) or the numerical formula (Numerical Formula 12D), energy of the triplet excitons generated in the first emitting layer is reduced, so that the organic EL device is expected to have a longer lifetime.

In the organic EL device of the exemplary embodiment, a triplet energy T₁(F1) of the compound contained in the first emitting layer and that emits fluorescence having a main peak wavelength of 500 nm or less also preferably satisfies a relationship of a numerical formula (Numerical Formula 14A) below, or also preferably satisfies a relationship of a numerical formula (Numerical Formula 14B) below.

$$2.6 eV > {T}_{1} ( F 1 ) \qquad ( Numerical Formula 14 A )$$

$$2.5 eV > {T}_{1} ( F 1 ) \qquad ( Numerical Formula 14 B )$$

The organic EL device has a longer lifetime by containing the compound that satisfies the numerical formula (Numerical Formula 14A) or the numerical formula (Numerical Formula 14B) in the first emitting layer.

In the organic EL device of the exemplary embodiment, a triplet energy T₁(F2) of the compound contained in the second emitting layer and that emits fluorescence having a main peak wavelength of 500 nm or less also preferably satisfies a relationship of a numerical formula (Numerical Formula 14C) below, or also preferably satisfies a relationship of a numerical formula (Numerical Formula 14D) below.

$$2.6 eV > {T}_{1} ( F 2 ) \qquad ( Numerical Formula 14 C )$$

$$2.5 eV > {T}_{1} ( F 2 ) \qquad ( Numerical Formula 14 D )$$

The second emitting layer contains the compound that satisfies the relationship of the numerical formula (Numerical Formula 14C) or the numerical formula (Numerical Formula 14D), so that the organic EL device has a longer lifetime.

In the organic EL device according to the present exemplary embodiment, the triplet energy T₁(H2) of the second host material preferably satisfies a relationship of a numerical formula (Numerical Formula 13) below.

$${T}_{1} ( H 2 ) \geq 1.9 eV . \qquad ( Numerical Formula 13 )$$

### Additional Layers of Organic EL Device

The organic EL device according to the exemplary embodiment may include one or more organic layer(s) in addition to the first emitting layer and the second emitting layer. Examples of the organic layer include at least one layer selected from the group consisting of a hole injecting layer, a hole transporting layer, an emitting layer, an electron injecting layer, an electron transporting layer, a hole blocking layer, and an electron blocking layer.

In the organic EL device of the exemplary embodiment, the organic layer may consist of the first emitting layer and the second emitting layer, however, may further includes at least one layer selected from the group consisting of a hole injecting layer, a hole transporting layer, an electron injecting layer, an electron transporting layer, a hole blocking layer, and an electron blocking layer.

It is preferable that the organic EL device of the exemplary embodiment further includes an anode and a cathode, the first emitting layer is provided between the anode and the cathode, and the second emitting layer is provided between the first emitting layer and the cathode.

The organic electroluminescence device of the exemplary embodiment also preferably includes the anode, the first emitting layer, the second emitting layer, and the cathode in this order.

### Hole Transporting Layer

The organic EL device of the exemplary embodiment preferably includes a hole transporting layer between the anode and the first emitting layer.

### Electron Transporting Layer

The organic EL device of the exemplary embodiment preferably includes an electron transporting layer between the second emitting layer and the cathode.

The FIGURE schematically shows an exemplary structure of the organic EL device of the exemplary embodiment.

An organic EL device 1 includes a light-transmissive substrate 2, an anode 3, a cathode 4, and an organic layer 10 provided between the anode 3 and the cathode 4. The organic layer 10 includes a hole injecting layer 6, a hole transporting layer 7, a first emitting layer 51, a second emitting layer 52, an electron transporting layer 8, and an electron injecting layer 9, which are sequentially laminated on the anode 3.

It should be noted that the scope of the invention is by no means limited by the arrangement of the organic EL device shown in the FIGURE. In the organic EL device with another arrangement, for instance, the organic layer includes the hole injecting layer, hole transporting layer, second emitting layer, first emitting layer, electron transporting layer, and electron injecting layer which are laminated on the anode in this order from the anode.

The organic EL device according to the exemplary embodiment may further include a third emitting layer.

It is preferable that the third emitting layer contains a third host material; the first host material, the second host material, and the third host material are mutually different, the third emitting layer at least contains a compound that emits fluorescence having a main peak wavelength of 500 nm or less; the compound that is contained in the first emitting layer and emits fluorescence having a main peak wavelength of 500 nm or less, the compound that is contained in the second emitting layer and emits fluorescence having a main peak wavelength of 500 nm or less, and the compound that is contained in the third emitting layer and emits fluorescence having a main peak wavelength of 500 nm or less are mutually the same or different; and a triplet energy T₁(H1) of the first host material and a triplet energy T₁(H3) of the third host material satisfy a relationship of a numerical formula (Numerical Formula 1A).

$${T}_{1} ( H 1 ) > {T}_{1} ( H 3 ) \qquad ( Numerical Formula 1 A )$$

When the organic EL device of the exemplary embodiment includes the third emitting layer, a triplet energy T₁(H2) of the second host material and a triplet energy T₁(H3) of the third host material preferably satisfy a relationship of a numerical formula (Numerical Formula 1B) below.

$${T}_{1} ( H 2 ) > {T}_{1} ( H 3 ) \qquad ( Numerical Formula 1 B )$$

In the organic EL device of the exemplary embodiment, the first emitting layer and the second emitting layer are preferably in direct contact with each other.

Herein, a layer arrangement that “the first emitting layer and the second emitting layer are in direct contact with each other” can include one of embodiments (LS₁), (LS2), and (LS3) below.

(LS₁) An embodiment in which a region containing both the first host material and the second host material is generated in a process of vapor-depositing the compound of the first emitting layer and vapor-depositing the compound of the second emitting layer, and is present on the interface between the first emitting layer and the second emitting layer.

(LS2) An embodiment in which in a case of containing an emitting compound in the first emitting layer and the second emitting layer, a region containing all of the first host material, the second host material and the emitting compound is generated in a process of vapor-depositing the compound of the first emitting layer and vapor-depositing the compound of the second emitting layer, and is present on the interface between the first emitting layer and the second emitting layer.

(LS3) An embodiment in which in a case of containing an emitting compound in the first emitting layer and the second emitting layer, a region containing the emitting compound, a region containing the first host material or a region containing the second host material is generated in a process of vapor-depositing the compound of the first emitting layer and vapor-depositing the compound of the second emitting layer, and is present on the interface between the first emitting layer and the second emitting layer.

When the organic EL device according to the exemplary embodiment includes the third emitting layer, it is preferable that the first emitting layer and the second emitting layer are in direct contact with each other and the second emitting layer and the third emitting layer are in direct contact with each other.

Herein, a layer arrangement that the second emitting layer and the third emitting layer are in direct contact with each other can include one of embodiments (LS4), (LS5) and (LS6) below.

(LS4) An embodiment in which a region containing both the second host material and the third host material is generated in a process of vapor-depositing the compound of the second emitting layer and vapor-depositing the compound of the third emitting layer, and is present on the interface between the second emitting layer and the third emitting layer.

(LS5) An embodiment in which in a case of containing an emitting compound in the second emitting layer and the third emitting layer, a region containing all of the second host material, the third host material and the emitting compound is generated in a process of vapor-depositing the compound of the second emitting layer and vapor-depositing the compound of the third emitting layer, and is present on the interface between the second emitting layer and the third emitting layer.

(LS6) An embodiment in which in a case of containing an emitting compound in the second emitting layer and the third emitting layer, a region containing the emitting compound, a region containing the second host material or a region containing the third host material is generated in a process of vapor-depositing the compound of the second emitting layer and vapor-depositing the compound of the third emitting layer, and is present on the interface between the second emitting layer and the third emitting layer.

It is also preferable that the organic EL device of the exemplary embodiment further includes a diffusion layer.

When the organic EL device of the exemplary embodiment includes the diffusion layer, it is preferable that the diffusion layer is interposed between the first emitting layer and the second emitting layer.

An arrangement of the organic EL device 1 will be further described. It should be noted that the reference numerals will be sometimes omitted below.

### Substrate

The substrate is used as a support for the organic EL device. For instance, glass, quartz, plastics and the like are usable for the substrate. A flexible substrate is also usable. The flexible substrate is a bendable substrate, which is exemplified by a plastic substrate. Examples of the material for the plastic substrate include polycarbonate, polyarylate, polyethersulfone, polypropylene, polyester, polyvinyl fluoride, polyvinyl chloride, polyimide, and polyethylene naphthalate. Moreover, an inorganic vapor deposition film is also usable.

### Anode

Metal, an alloy, an electrically conductive compound, a mixture thereof, or the like having a large work function (specifically, 4.0 eV or more) is preferably used as the anode formed on the substrate. Specific examples of the material include ITO (Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the organic layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, a mixture thereof, or the like having a small work function (specifically, 3.8 eV or less) for the cathode. Examples of the material for the cathode include elements belonging to Groups 1 and 2 in the periodic table of the elements, specifically, the alkali metal such as lithium (Li) and cesium (Cs), the alkaline earth metal such as magnesium (Mg), calcium (Ca) and strontium (Sr), alloys (e.g., MgAg and AlLi) including the alkali metal or the alkaline earth metal, the rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method and the like.

### Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the highly hole-injectable substance further include: an aromatic amine compound, which is a low-molecule organic compound, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,3-f:20,30-h]quinoxaline-2,3,6,7,10,11-hexacarbonitrile (HAT-CN).

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine](abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) are also usable.

### Hole Transporting Layer

The hole transporting layer is a layer containing a highly hole-transporting substance. An aromatic amine compound, carbazole derivative, anthracene derivative and the like are usable for the hole transporting layer. Specific examples of a material for the hole transporting layer include an aromatic amine compound such as 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(V·s) or more.

For the hole transporting layer, a carbazole derivative such as CBP, 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be not only a single layer but also a laminate of two or more layers formed of the above substance(s).

### Electron Transporting Layer

The electron transporting layer is a layer containing a highly electron-transporting substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinato)aluminum (abbreviation: Almq₃), bis(10-hydroxybenzo[h]quinolinato)beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In the exemplary embodiment, a benzimidazole compound is suitably usable. The above-described substances mostly have an electron mobility of 10⁻⁶ cm²/Vs or more. It should be noted that any substance other than the above substance may be used for the electron transporting layer as long as the substance exhibits a higher electron transportability than the hole transportability. The electron transporting layer may be provided in the form of a single layer or a laminate of two or more layers of the above substance(s).

Further, a high polymer compound is usable for the electron transporting layer. For instance, poly[(9,9-dihexylfluorene-2,7-diyl)-co-(pyridine-3,5-diyl)] (abbreviation: PF-Py), poly[(9,9-dioctylfluorene-2,7-diyl)-co-(2,2′-bipyridine-6,6′-diyl)] (abbreviation: PF-BPy) and the like are usable.

### Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Layer Formation Method

A method for forming each layer of the organic EL device in the present exemplary embodiment is subject to no limitation except for the above particular description. However, known methods of dry film-forming such as vacuum deposition, sputtering, plasma or ion plating and wet film-forming such as spin coating, dipping, flow coating or ink-jet are applicable.

### Film Thickness

A film thickness of each of the organic layers of the organic EL device in the exemplary embodiment is not limited unless otherwise specified in the above. In general, the thickness preferably ranges from several nanometers to 1 μm because excessively small film thickness is likely to cause defects (e.g. pin holes) and excessively large thickness leads to the necessity of applying high voltage and consequent reduction in efficiency.

### First Host Material, Second Host Material, and Third Host Material

In the organic EL device of the exemplary embodiment, examples of the first host material, the second host material, and the third host material includes the first compound represented by a formula (1), formula (1X), formula (12X), formula (13X), formula (14X), formula (15X) or formula (16X) below and the second compound represented by a formula (2) below. Moreover, the first compound is usable as the first host material and the second host material. In this case, the compound represented by the formula (1) or the compound represented by the formula (1X), formula (12X), formula (13X), formula (14X), formula (15X) or formula (16X) which are used as the second host material is sometimes referred to as the second compound for convenience.

### First Compound

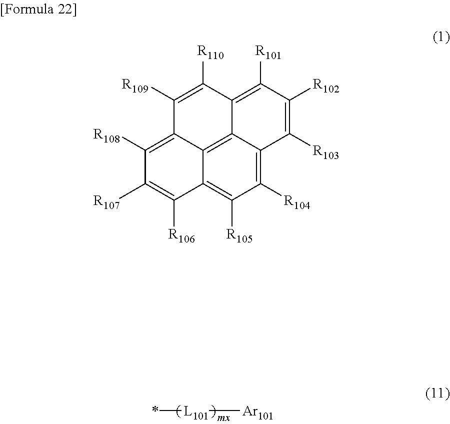

In the formula (1):
R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11) above;
at least one of R₁₀₁ to R₁₁₀ is a group represented by the formula (11);
when a plurality of groups represented by the formula (11) are present, the plurality of groups represented by the formula (11) are mutually the same or different;
L₁₀₁ is a single bond, a substituted or unsubstituted arylene group having 6 to 50 ring carbon atoms, or a substituted or unsubstituted divalent heterocyclic group having 5 to 50 ring atoms;
Ar₁₀₁ is a substituted or unsubstituted aryl group having 6 to 50 ring carbon atoms, or a substituted or unsubstituted heterocyclic group having 5 to 50 ring atoms;
mx is 0, 1, 2, 3, 4 or 5; and
when two or more L₁₀₁ are present, two or more L₁₀₁ are mutually the same or different;
when two or more Ar₁₀₁ are present, two or more Ar₁₀₁ are mutually the same or different; and
* in the formula (11) represents a bonding position to a pyrene ring in the formula (1).

In the first compound according to the exemplary embodiment, R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In the organic EL device according to the exemplary embodiment, the group represented by the formula (11) is preferably a group represented by a formula (111) below.

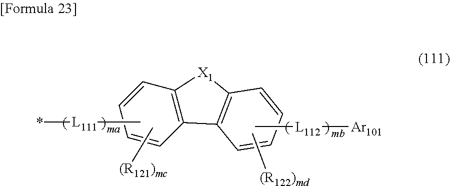

In the formula (111):
X₁ is CR₁₂₃R₁₂₄, an oxygen atom, a sulfur atom, or NR₁₂₅;
L₁₁₁ and L₁₁₂ are each independently a single bond, a substituted or unsubstituted arylene group having 6 to 50 ring carbon atoms, or a substituted or unsubstituted divalent heterocyclic group having 5 to 50 ring atoms;
ma is 0, 1, 2, 3, or 4;
mb is 0, 1, 2, 3, or 4;
ma+mb is 0, 1, 2, 3, or 4;
Ar₁₀₁ represents the same as Ar₁₀₁ in the formula (11);
R₁₂₁, R₁₂₂, R₁₂₃, R₁₂₄, and R₁₂₅ are each dependently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
mc is 3;
three R₁₂₁ are mutually the same or different;
md is 3; and
three R₁₂₂ are mutually the same or different.

Among positions *1 to *8 of carbon atoms in the cyclic structure represented by a formula (111a) below in the group represented by the formula (111), L₁₁₁ is bonded to one of positions *1 to *4, R₁₂₁ is bonded to three positions of the rest of *1 to *4, L₁₁₂ is bonded to one of positions *5 to *8, and R₁₂₂ is bonded to three positions of the rest of *5 to *8.

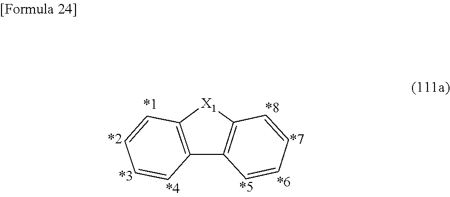

For instance, in the group represented by the formula (111), when L₁₁₁ and L₁₁₂ are bonded to *2 and *7 positions, respectively, of the carbon atom of the cyclic structure represented by the formula (111a), the group represented by the formula (111) is represented by a formula (111 b) below.

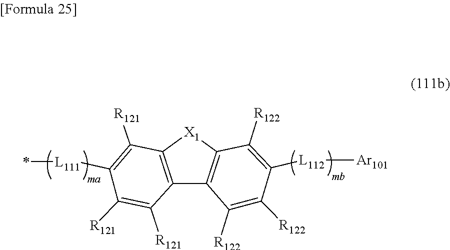

In the formula (111b): X₁, L₁₁₁, L₁₁₂, ma, mb, Ar₁₀₁, R₁₂₁, R₁₂₂, R₁₂₃, R₁₂₄, and R₁₂₅ each independently represent the same as X₁, L₁₁₁, L₁₁₂, ma, mb, Ar₁₀₁, R₁₂₁, R₁₂₂, R₁₂₃, R₁₂₄, and R₁₂₅ in the formula (111);
a plurality of R₁₂₁ are mutually the same or different; and
a plurality of R₁₂₂ are mutually the same or different.

In the organic EL device of the exemplary embodiment, the group represented by the formula (111) is preferably a group represented by the formula (111b).

In the organic EL device according to the exemplary embodiment, it is preferable that ma is 0, 1, or 2, and
mb is 0, 1, or 2.

In the organic EL device according to the exemplary embodiment, it is preferable that ma is 0 or 1, and
mb is 0 or 1.

In the organic EL device according to the exemplary embodiment, Ar₁₀₁ is preferably a substituted or unsubstituted aryl group having 6 to 50 ring carbon atoms.

In the organic EL device according to the exemplary embodiment, Ar₁₀₁ is preferably a substituted or unsubstituted phenyl group, a substituted or unsubstituted naphthyl group, a substituted or unsubstituted biphenyl group, a substituted or unsubstituted terphenyl group, a substituted or unsubstituted pyrenyl group, a substituted or unsubstituted phenanthryl group, or a substituted or unsubstituted fluorenyl group.

In the organic EL device according to the exemplary embodiment, Ar₁₀₁ is also preferably a group represented by a formula (12), a formula (13), or a formula (14) below.

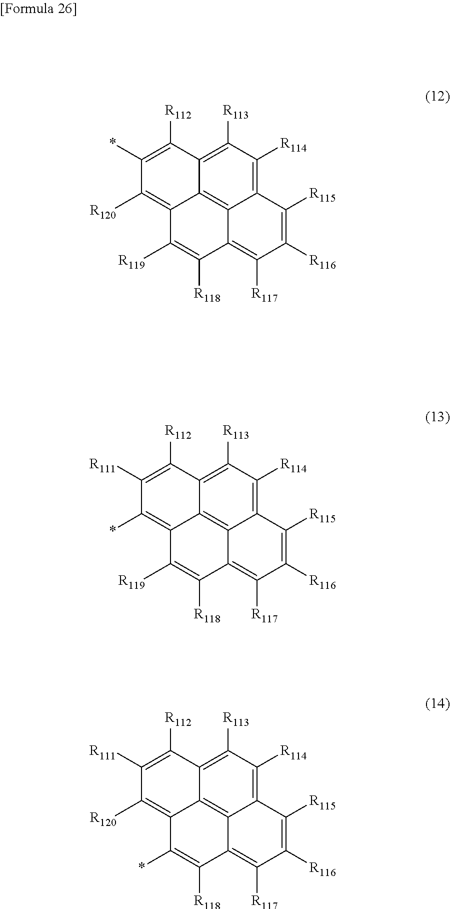

In the formulae (12), (13), and (14):
R₁₁₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₁₂₄, a group represented by —COOR₁₂₅ a halogen atom, a cyano group, a nitro group, a substituted or unsubstituted aryl group having 6 to 50 ring carbon atoms, or a substituted or unsubstituted heterocyclic group having 5 to 50 ring atoms; and
* in the formulae (12), (13) and (14) represents a bonding position to L₁₀₁ in the formula (11) or a bonding position to L₁₁₂ in the formula (111) or (111b).

In the organic EL device according to the exemplary embodiment, the first compound is preferably represented by a formula (101) below.

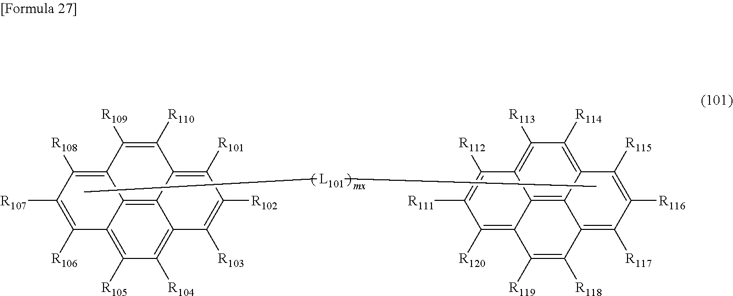

In the formula (101):
R₁₀₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
one of R₁₀₁ to R₁₁₀ represents a bonding position to L₁₀₁, and one of R₁₁₁ to R₁₂₀ represents a bonding position to L₁₀₁;
L₁₀₁ is a single bond, a substituted or unsubstituted arylene group having 6 to 50 ring carbon atoms, or a substituted or unsubstituted divalent heterocyclic group having 5 to 50 ring atoms;
mx is 0, 1, 2, 3, 4 or 5; and
when two or more L₁₀₁ are present, the two or more L₁₀₁ are mutually the same or different.

In the organic EL device according to the exemplary embodiment, L₁₀₁ is preferably a single bond or a substituted or unsubstituted arylene group having 6 to 50 ring carbon atoms.

In the organic EL device according to the exemplary embodiment, the first compound is preferably represented by a formula (102) below.

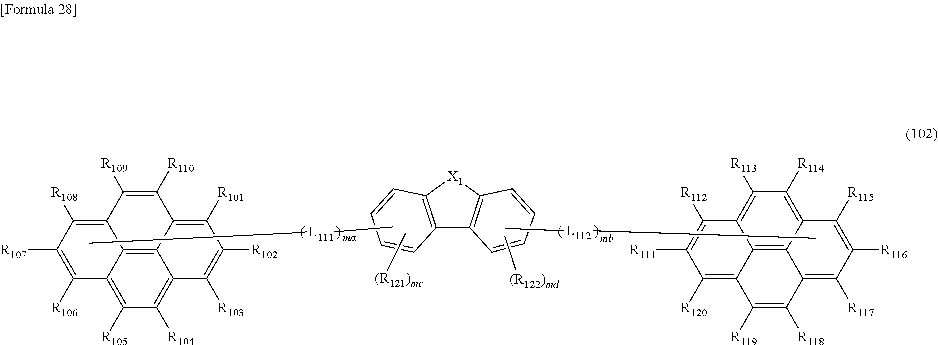

In the formula (102):
R₁₀₁ to R₁₂₀ each independently represent the same as R₁₀₁ to R₁₂₀ of the formula (101);
one of R₁₀₁ to R₁₁₀ represents a bonding position to L₁₁₁, and one of R₁₁₁ to R₁₂₀ represents a bonding position to L₁₁₂;
X₁ is CR₁₂₃R₁₂₄, an oxygen atom, a sulfur atom, or NR₁₂₅;
L₁₁₁ and L₁₁₂ are each independently a single bond, a substituted or unsubstituted arylene group having 6 to 50 ring carbon atoms, or a substituted or unsubstituted divalent heterocyclic group having 5 to 50 ring atoms;
ma is 0, 1, 2, 3, or 4;
mb is 0, 1, 2, 3, or 4;
ma+mb is 0, 1, 2, 3, or 4;
R₁₂₁, R₁₂₂, R₁₂₃, R₁₂₄, and R₁₂₅ are each dependently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
mc is 3;
three R₁₂₁ are mutually the same or different;
md is 3; and
three R₁₂₂ are mutually the same or different.

In the compound represented by the formula (102), it is preferable that: ma is 0, 1, or 2, and mb is 0, 1, or 2.

In the compound represented by the formula (102), it is preferable that: ma is 0 or 1, and mb is 0 or 1.

In the organic EL device according to the exemplary embodiment, two or more of R₁₀₁ to R₁₁₀ are preferably groups represented by the formula (11).

In the organic EL device according to the exemplary embodiment, it is preferable that two or more of R₁₀₁ to R₁₁₀ are groups represented by the formula (11) and Ar₁₀₁ is a substituted or unsubstituted aryl group having 6 to 50 ring carbon atoms.

In the organic EL device according to the exemplary embodiment, it is preferable that Ar₁₀₁ is not a substituted or unsubstituted pyrenyl group;
L₁₀₁ is not a substituted or unsubstituted pyrenylene group; and
the substituted or unsubstituted aryl group having 6 to 50 ring carbon atoms for R₁₀₁ to R₁₁₀ not being the group represented by the formula (11) is not a substituted or unsubstituted pyrenyl group.

In the organic EL device according to the exemplary embodiment, it is preferable that R₁₀₁ to R₁₁₀ not being the group represented by the formula (11) are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the organic EL device according to the exemplary embodiment, it is preferable that R₁₀₁ to R₁₁₀ not being the group represented by the formula (11) are each independently a hydrogen atom, a substituted or unsubstituted alkyl group having 1 to 50 carbon atoms, or a substituted or unsubstituted cycloalkyl group having 3 to 50 ring carbon atoms.

In the organic EL device according to the exemplary embodiment, R₁₀₁ to R₁₁₀ not being the group represented by the formula (11) are each preferably a hydrogen atom.

### Compound Represented by Formula (1X)

In the organic EL device of the exemplary embodiment, the first compound is also preferably represented by a formula (1X) below.

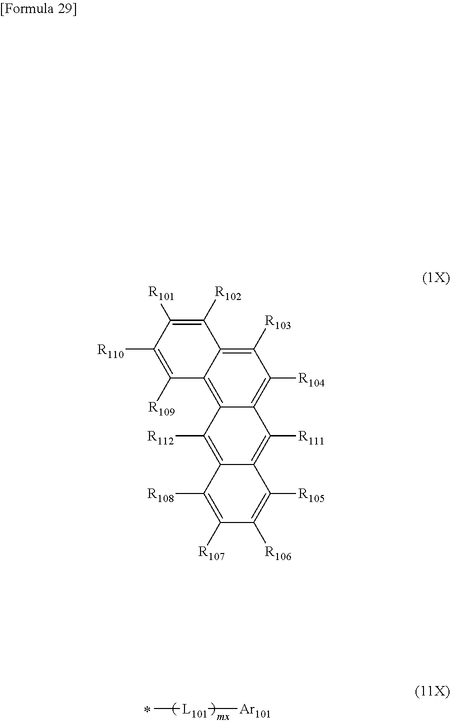

In the formula (1X):
R₁₀₁ to R₁₁₂ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by a formula (11X) above;
at least one of R₁₀₁ to R₁₁₂ is the group represented by the formula (11X);
when a plurality of groups represented by the formula (11X) are present, the plurality of groups represented by the formula (11X) are mutually the same or different;
L₁₀₁ is a single bond, a substituted or unsubstituted arylene group having 6 to 50 ring carbon atoms, or a substituted or unsubstituted divalent heterocyclic group having 5 to 50 ring atoms;
Ar₁₀₁ is a substituted or unsubstituted aryl group having 6 to 50 ring carbon atoms, or a substituted or unsubstituted heterocyclic group having 5 to 50 ring atoms;
mx is 1, 2, 3, 4 or 5;
when two or more L₁₀₁ are present, two or more L₁₀₁ are mutually the same or different;
when two or more Ar₁₀₁ are present, two or more Ar₁₀₁ are mutually the same or different; and
* in the formula (11X) represents a bonding position to a benz[a]anthracene ring in the formula (1X).

In the organic EL device of the exemplary embodiment, the group represented by the formula (11X) is preferably a group represented by a formula (111X) below.

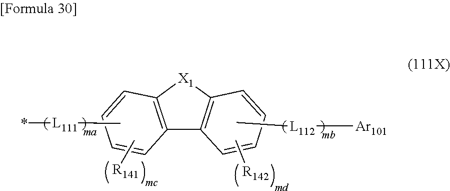

In the formula (111X);
X₁ is CR₁₄₃R₁₄₄, an oxygen atom, a sulfur atom, or NR₁₄₅;
L₁₁₁ and L₁₁₂ are each independently a single bond, a substituted or unsubstituted arylene group having 6 to 50 ring carbon atoms, or a substituted or unsubstituted divalent heterocyclic group having 5 to 50 ring atoms;
ma is 1, 2, 3, or 4;
mb is 1, 2, 3, or 4;
ma+mb is 2, 3, or 4;
Ar₁₀₁ represents the same as Ar₁₀₁ in the formula (11);
R₁₄₁, R₁₄₂, R₁₄₃, R₁₄₄, and R₁₄₅ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
mc is 3;
three R₁₄₁ are mutually the same or different;
md is 3; and
three R₁₄₂ are mutually the same or different.

Among positions *1 to *8 of carbon atoms in a cyclic structure represented by a formula (111aX) below in the group represented by the formula (111X), L₁₁₁ is bonded to one of the positions *1 to *4, R₁₄₁ is bonded to each of three positions of the rest of *1 to *4, L₁₁₂ is bonded to one of the positions *5 to *8, and R₁₄₂ is bonded to each of three positions of the rest of *5 to *8.

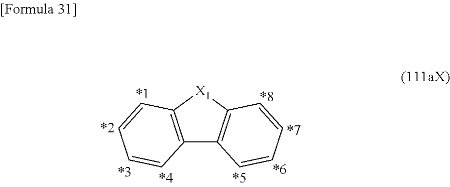

For instance, in the group represented by the formula (111X), when L₁₁₁ is bonded to a carbon atom at *2 in the cyclic structure represented by the formula (111 aX) and L₁₁₂ is bonded to a carbon atom at *7 in the cyclic structure represented by the formula (111aX), the group represented by the formula (111X) is represented by a formula (111bX) below.

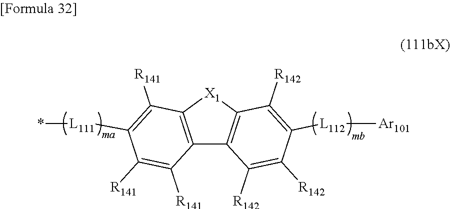

In the formula (111 bX):
X₁, L₁₁₁, L₁₁₂, ma, mb, Ar₁₀₁, R₁₄₁, R₁₄₂, R₁₄₃, R₁₄₄ and R₁₄₅ each independently represent the same as X₁, L₁₁₁, L₁₁₂, ma, mb, Ar₁₀₁, R₁₄₁, R₁₄₂, R₁₄₃, R₁₄₄ and R₁₄₅ in the formula (111X);
a plurality of R₁₄₁ are mutually the same or different; and
a plurality of R₁₄₂ are mutually the same or different.

In the organic EL device of the exemplary embodiment, the group represented by the formula (111X) is preferably a group represented by the formula (111bX).

In the compound represented by the formula (1X), it is preferable that ma is 1 or 2 and mb is 1 or 2.

In the compound represented by the formula (1X), it is preferable that ma is 1 and mb is 1.

In the compound represented by the formula (1X), Ar₁₀₁ is preferably a substituted or unsubstituted aryl group having 6 to 50 ring carbon atoms.

In the compound represented by the formula (1X), Ar₁₀₁ is preferably a substituted or unsubstituted phenyl group, a substituted or unsubstituted naphthyl group, a substituted or unsubstituted biphenyl group, a substituted or unsubstituted terphenyl group, a substituted or unsubstituted benz[a]anthryl group; a substituted or unsubstituted pyrenyl group, a substituted or unsubstituted phenanthryl group, or a substituted or unsubstituted fluorenyl group.

The compound represented by the formula (1X) is also preferably represented by a formula (101X) below.

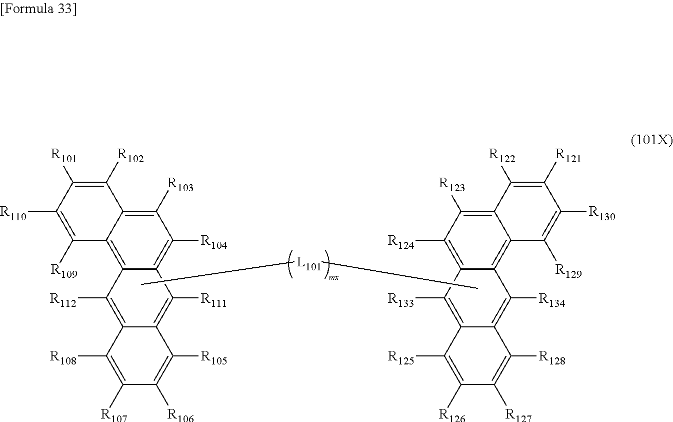

In the formula (101X):
one of R₁₁₁ and R₁₁₂ represents a bonding position to L₁₀₁ and one of R₁₃₃ and R₁₃₄ represents a bonding position to L₁₀₁;
R₁₁₁ or R₁₁₂ that is not a bonding position to R₁₀₁ to R₁₁₀, R₁₂₁ to R₁₃₀, and L₁₀₁, and R₁₃₃ or R₁₃₄ that is not a bonding position to L₁₀₁ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₁₀₁ is a single bond, a substituted or unsubstituted arylene group having 6 to 50 ring carbon atoms, or a substituted or unsubstituted divalent heterocyclic group having 5 to 50 ring atoms;
mx is 1, 2, 3, 4 or 5; and
when two or more L₁₀₁ are present, the two or more L₁₀₁ are mutually the same or different.

In the compound represented by the formula (1X), L₁₀₁ is preferably a single bond or a substituted or unsubstituted arylene group having 6 to 50 ring carbon atoms.

The compound represented by the formula (1X) is also preferably represented by a formula (102X) below.

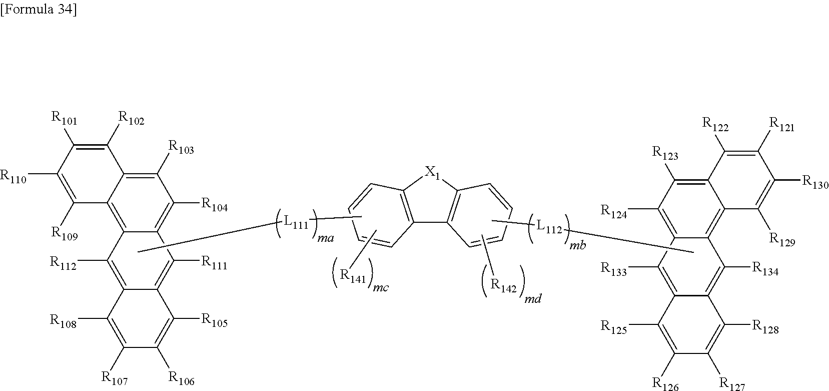

In the formula (102X):
one of R₁₁ and R₁₁₂ represents a bonding position to L₁₁₁ and one of R₁₃₃ and R₁₃₄ represents a bonding position to L₁₁₂;
R₁₁₁ or R₁₁₂ that is not a bonding position to R₁₀₁ to R₁₁₀, R₁₂₁ to R₁₃₀, and L₁₁₁, and R₁₃₃ or R₁₃₄ that is not a bonding position to R₁₁₁ or R₁₁₂ and L₁₁₂ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
X₁ is CR₁₄₃R₁₄₄, an oxygen atom, a sulfur atom, or NR₁₄₅;
L₁₁₁ and L₁₁₂ are each independently a single bond, a substituted or unsubstituted arylene group having 6 to 50 ring carbon atoms, or a substituted or unsubstituted divalent heterocyclic group having 5 to 50 ring atoms;
ma is 1, 2, 3, or 4;
mb is 1, 2, 3, or 4;
ma+mb is 2, 3, 4, or 5;
R₁₄₁, R₁₄₂, R₁₄₃, R₁₄₄, and R₁₄₅ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
mc is 3;
three R₁₄₁ are mutually the same or different;
md is 3; and
three R₁₄₂ are mutually the same or different.

In the compound represented by the formula (1X), it is preferable that ma is 1 or 2 and mb is 1 or 2 in the formula (102X).

In the compound represented by the formula (1X), it is preferable that ma is 1 and mb is 1 in the formula (102X).

In the compound represented by the formula (1X), the group represented by the formula (11X) is also preferably a group represented by a formula (11AX) or a group represented by a formula (11BX).

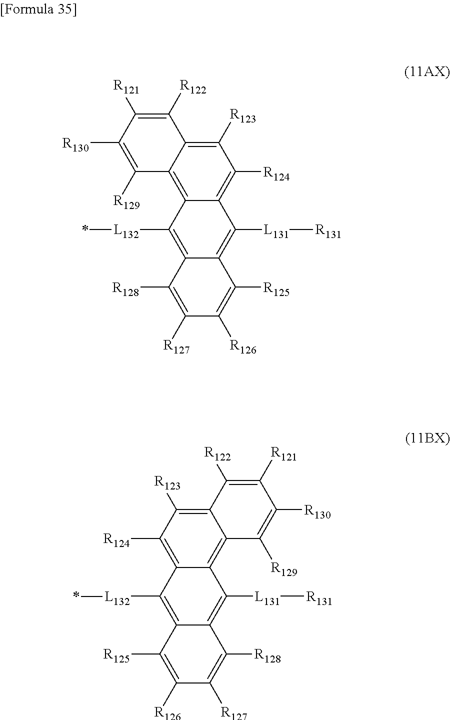

In the formulae (11AX) and (11BX):
R₁₂₁ to R₁₃₁ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms,
when a plurality of groups represented by the formula (11AX) are present, the plurality of groups represented by the formula (11AX) are mutually the same or different;
when a plurality of groups represented by the formula (11BX) are present, the plurality of groups represented by the formula (11BX) are mutually the same or different;
L₁₃₁ and L₁₃₂ are each independently a single bond, a substituted or unsubstituted arylene group having 6 to 50 ring carbon atoms, or a substituted or unsubstituted divalent heterocyclic group having 5 to 50 ring atoms; and
* in the formulae (11AX) and (11BX) each represents a bonding position to a benz[a]anthracene ring in the formula (1X).

The compound represented by the formula (1X) is also preferably represented by a formula (103X).

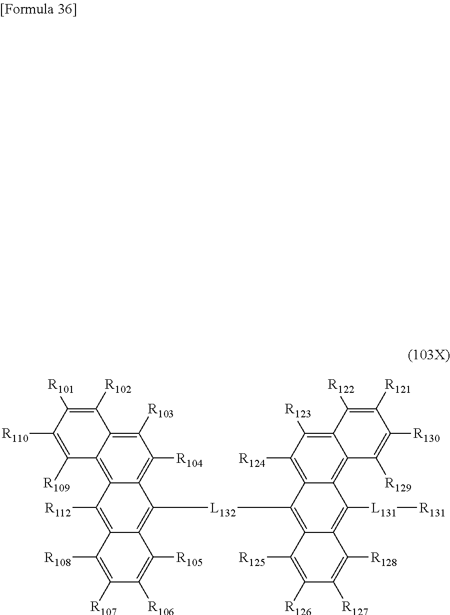

In the formula (103X):
R₁₀₁ to R₁₁₀ and R₁₁₂ respectively represent the same as R₁₀₁ to R₁₁₀ and R₁₁₂ in the formula (1X); and
R₁₂₁ to R₁₃₁, L₁₃₁ and L₁₃₂ respectively represent the same as R₁₂₁ to R₁₃₁, L₁₃₁ and L₁₃₂ in the formula (11BX).

In the compound represented by the formula (1X), L₁₃₁ is preferably a substituted or unsubstituted arylene group having 6 to 50 ring carbon atoms.

In the compound represented by the formula (1X), L₁₃₂ is preferably a substituted or unsubstituted arylene group having 6 to 50 ring carbon atoms.

In the compound represented by the formula (1X), two or more of R₁₀₁ to R₁₁₂ are preferably the groups represented by the formula (11).

In the compound represented by the formula (1X), it is preferable that two or more of R₁₀₁ to R₁₁₂ are the groups represented by the formula (11X) and Ar₁₀₁ in the formula (11X) is a substituted or unsubstituted aryl group having 6 to 50 ring carbon atoms.

In the compound represented by the formula (1X), it is also preferable that Ar₁₀₁ is not a substituted or unsubstituted benz[a]anthryl group,
L₁₀₁ is not a substituted or unsubstituted benz[a]anthrylene group, and
a substituted or unsubstituted aryl group having 6 to 50 ring carbon atoms as R₁₀₁ to R₁₁₀ that are not the group represented by the formula (11X) is not a substituted or unsubstituted benz[a]anthryl group.

In the compound represented by the formula (1X), R₁₀₁ to R₁₁₂ that are not the group represented by the formula (11X) are each independently preferably a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the compound represented by the formula (1X), R₁₀₁ to R₁₁₂ that are not the group represented by the formula (11X) are preferably a hydrogen atom, a substituted or unsubstituted alkyl group having 1 to 50 carbon atoms, or a substituted or unsubstituted cycloalkyl group having 3 to 50 ring carbon atoms.

In the compound represented by the formula (1X), R₁₀₁ to R₁₁₂ that are not the group represented by the formula (11X) are each preferably a hydrogen atom.

### Compound Represented by Formula (12X)

In the organic EL device of the exemplary embodiment, the first compound is also preferably the compound represented by the formula (12X).

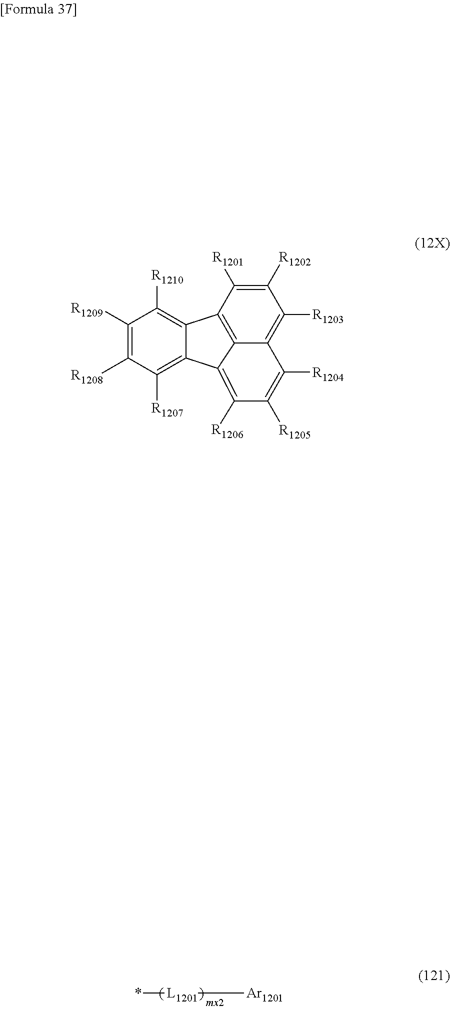

In the formula (12X):
at least one combination of adjacent two or more of R₁₂₀₁ to R₁₂₁₀ are mutually bonded to form a substituted or unsubstituted monocyclic ring, or mutually bonded to form a substituted or unsubstituted fused ring;
R₁₂₀₁ to R₁₂₁₀ neither forming the substituted or unsubstituted monocyclic ring nor forming the substituted or unsubstituted fused ring each independently represent a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (121);
a substituent for substituting the substituted or unsubstituted monocyclic ring, a substituent for substituting a substituted or unsubstituted fused ring, and at least one of R₁₂₀₁ to R₁₂₁₀ are a group represented by the formula (121);
when a plurality of groups represented by the formula (121) are present, the plurality of groups represented by the formula (121) are mutually the same or different;
L₁₂₀₁ is a single bond, a substituted or unsubstituted arylene group having 6 to 50 ring carbon atoms, or a substituted or unsubstituted divalent heterocyclic group having 5 to 50 ring atoms; and
Ar₁₂₀₁ is a substituted or unsubstituted aryl group having 6 to 50 ring carbon atoms, or a substituted or unsubstituted heterocyclic group having 5 to 50 ring atoms;
mx2 is 0, 1, 2, 3, 4, or 5;
when two or more L₁₂₀₁ are present, the two or more L₁₂₀₁ are mutually the same or different;
when two or more Ar₁₂₀₁ are present, the two or more Ar₁₂₀₁ are mutually the same or different; and
* in the formula (121) represents a bonding position to a ring represented by the formula (12X).

In the formula (12X), combinations of adjacent two of R₁₂₀₁ to R₁₂₁₀ refer to a combination of R₁₂₀₁ and R₁₂₀₂, a combination of R₁₂₀₂ and R₁₂₀₃, a combination of R₁₂₀₃ and R₁₂₀₄, a combination of R₁₂₀₄ and R₁₂₀₅, a combination of R₁₂₀₅ and R₁₂₀₆, a combination of R₁₂₀₇ and R₁₂₀₈, a combination of R₁₂₀₈ and R₁₂₀₉, and a combination of R₁₂₀₉ and R₁₂₁₀.

### Compound Represented by Formula (13X)

In the organic EL device of the exemplary embodiment, the first compound is also preferably a compound represented by a formula (13X).

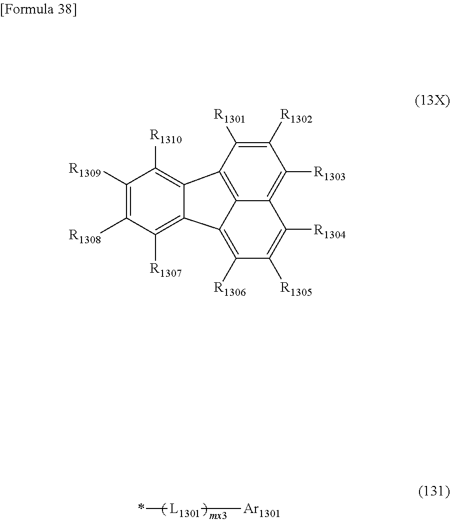

In the formula (13X):
R₁₃₀₁ to R₁₃₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (131);
at least one of R₁₃₀₁ to R₁₃₁₀ is a group represented by the formula (131);
when a plurality of groups represented by the formula (131) are present, the plurality of groups represented by the formula (131) are mutually the same or different;
L₁₃₀₁ is a single bond, a substituted or unsubstituted arylene group having 6 to 50 ring carbon atoms, or a substituted or unsubstituted divalent heterocyclic group having 5 to 50 ring atoms;
Ar₁₃₀₁ is a substituted or unsubstituted aryl group having 6 to 50 ring carbon atoms, or a substituted or unsubstituted heterocyclic group having 5 to 50 ring atoms;
mx3 is 0, 1, 2, 3, 4, or 5;
when two or more L₁₃₀₁ are present, the two or more L₁₃₀₁ are mutually the same or different;
when two or more Ar₁₃₀₁ are present, two or more Ar₁₃₀₁ are mutually the same or different; and
* in the formula (131) represents a bonding position to a fluoranthene ring in the formula (13X).

In the organic EL device of the exemplary embodiment, combinations of adjacent two or more of R₁₃₀₁ to R₁₃₁₀ that are not the group represented by the formula (131) are not bonded to each other. In the formula (13X), combinations of adjacent two of R₁₃₀₁ to R₁₃₁₀ refer to a combination of R₁₃₀₁ and R₁₃₀₂, a combination of R₁₃₀₂ and R₁₃₀₃, a combination of R₁₃₀₃ and R₁₃₀₄, a combination of R₁₃₀₄ and R₁₃₀₅, a combination of R₁₃₀₅ and R₁₃₀₆, a combination of R₁₃₀₇ and R₁₃₀₈, a combination of R₁₃₀₈ and R₁₃₀₉, and a combination of R₁₃₀₉ and R₁₃₁₀.

### Compound Represented by Formula (14X)

In the organic EL device of the exemplary embodiment, the first compound is also preferably a compound represented by a formula (14X) below.

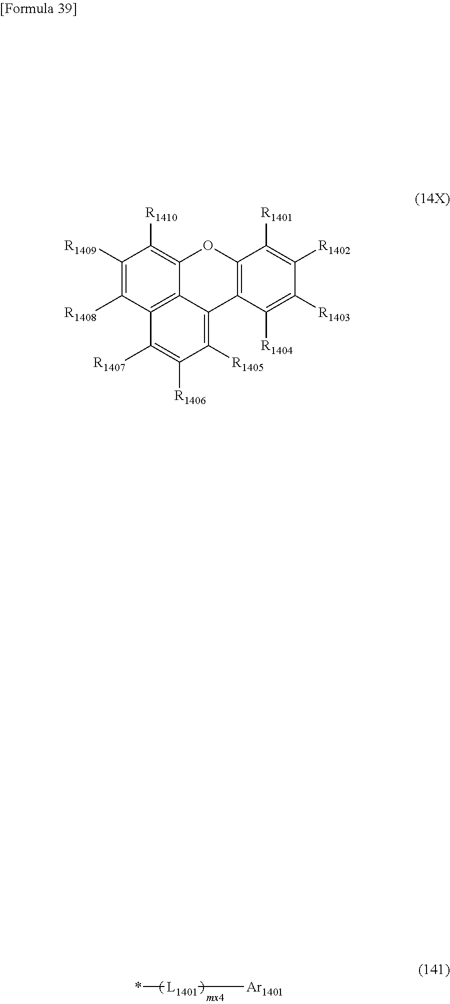

In the formula (14X):
R₁₄₀₁ to R₁₄₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (141);
at least one of R₁₄₀₁ to R₁₄₁₀ is a group represented by the formula (141);
when a plurality of groups represented by the formula (141) are present, the plurality of groups represented by the formula (141) are mutually the same or different;
L₁₄₀₁ is a single bond, a substituted or unsubstituted arylene group having 6 to 50 ring carbon atoms, or a substituted or unsubstituted divalent heterocyclic group having 5 to 50 ring atoms;
Ar₁₄₀₁ is a substituted or unsubstituted aryl group having 6 to 50 ring carbon atoms, or a substituted or unsubstituted heterocyclic group having 5 to 50 ring atoms;
mx4 is 0, 1, 2, 3, 4, or 5;
when two or more L₁₄₀₁ are present, the two or more L₁₄₀₁ are mutually the same or different;
when two or more Ar₁₄₀₁ are present, the two or more Ar₁₄₀₁ are mutually the same or different; and
* in the formula (141) represents a bonding position to a ring represented by the formula (14X).

### Compound Represented by Formula (15X)

In the organic EL device of the exemplary embodiment, the first compound is also preferably a compound represented by a formula (15X) below.

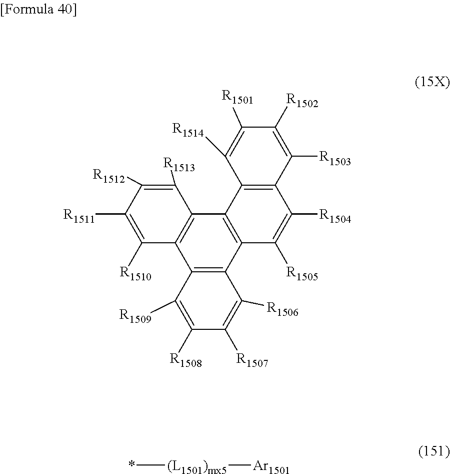

In the formula (15X):
R₁₅₀₁ to R₁₅₁₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (151);
at least one of R₁₅₀₁ to R₁₅₁₄ is a group represented by the formula (151);
when a plurality of group represented by the formula (151) are present, the plurality of groups represented by the formula (151) are mutually the same or different;
L₁₅₀₁ is a single bond, a substituted or unsubstituted arylene group having 6 to 50 ring carbon atoms, or a substituted or unsubstituted divalent heterocyclic group having 5 to 50 ring atoms;
Ar₁₅₀₁ is a substituted or unsubstituted aryl group having 6 to 50 ring carbon atoms, or a substituted or unsubstituted heterocyclic group having 5 to 50 ring atoms;
mx5 is 0, 1, 2, 3, 4, or 5;
when two or more L₁₅₀₁ are present, the two or more L₁₅₀₁ are mutually the same or different;
when two or more Ar₁₅₀₁ are present, the two or more Ar₁₅₀₁ are mutually the same or different; and
* in the formula (151) represents a bonding position to a ring represented by the formula (15X).

### Compound Represented by Formula (16X)

In the organic EL device of the exemplary embodiment, the first compound is also preferably is a compound represented by a formula (16X) below.

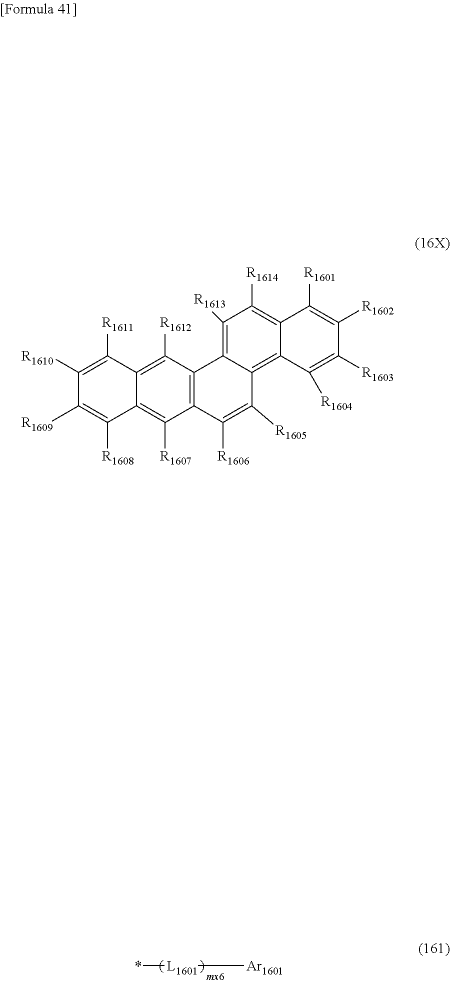

In the formula (16X):
R₁₆₀₁ to R₁₆₁₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (161);
at least one of R₁₆₀₁ to R₁₆₁₄ is a group represented by the formula (161);
when a plurality of group represented by the formula (161) are present, the plurality of groups represented by the formula (161) are mutually the same or different;
L₁₆₀₁ is a single bond, a substituted or unsubstituted arylene group having 6 to 50 ring carbon atoms, or a substituted or unsubstituted divalent heterocyclic group having 5 to 50 ring atoms;
Ar₁₆₀₁ is a substituted or unsubstituted aryl group having 6 to 50 ring carbon atoms, or a substituted or unsubstituted heterocyclic group having 5 to 50 ring atoms;
mx6 is 0, 1, 2, 3, 4, or 5;
when two or more L₁₆₀₁ are present, the two or more L₁₆₀₁ are mutually the same or different;
when two or more Ar₁₆₀₁ are present, the two or more Ar₁₆₀₁ are mutually the same or different; and
* in the formula (161) represents a bonding position to a ring represented by the formula (16X).

In the organic EL device of the exemplary embodiment, it is also preferable that the first host material has, in a molecule, a linking structure including a benzene ring and a naphthalene ring linked to each other with a single bond, in which the benzene ring and the naphthalene ring in the linking structure are each independently fused or not fused with a further monocyclic ring or fused ring, and the benzene ring and the naphthalene ring in the linking structure are further linked to each other by cross-linking at at least one site other than the single bond.

Since the first host material has the linking structure including such cross-linking, it can be expected to suppress deterioration in chromaticity of the organic EL device.

The first host material in the above case is only required to have a linking structure as the minimum unit in a molecule, the linking structure including a benzene ring and a naphthalene ring linked to each other with a single bond (referred to as a benzene-naphthalene linking structure in some cases), the linking structure being as represented by a formula (X1) or a formula (X2) below. The benzene ring may be fused with a further monocyclic ring or fused ring, and the naphthalene ring may be fused with a further monocyclic ring or fused ring. For example, also in a case where the first host material has, in a molecule, a linking structure including a naphthalene ring and a naphthalene ring linked to each other with a single bond (referred to as a naphthalene-naphthalene linking structure in some cases) and being as represented by a formula (X3), a formula (X4), or a formula (X5) below, the naphthalene-naphthalene linking structure is regarded as including the benzene-naphthalene linking structure since one of the naphthalene rings includes a benzene ring.

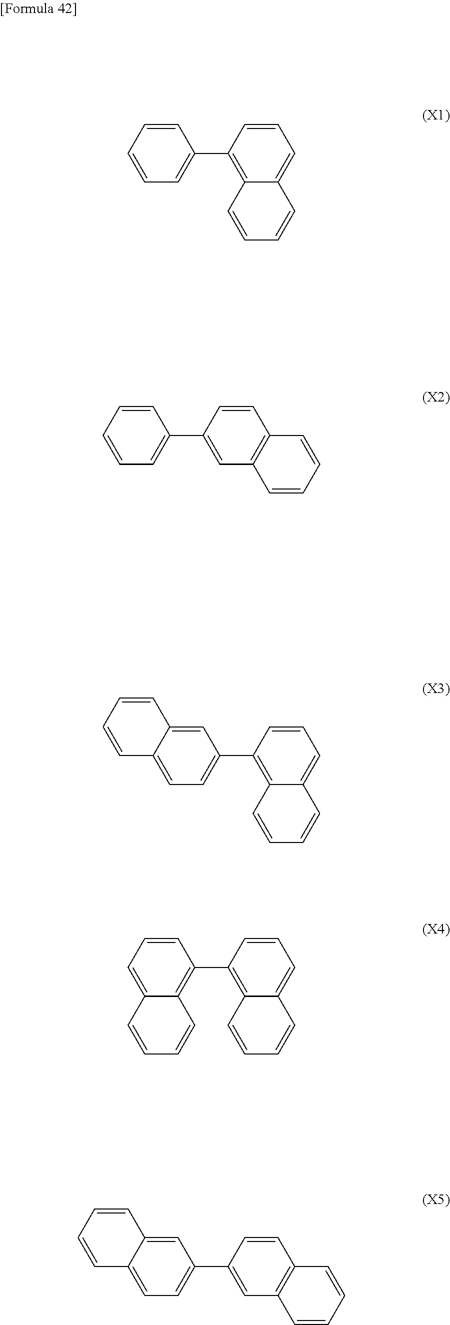

In the organic EL device according to the exemplary embodiment, the cross-linking also preferably includes a double bond. Specifically, the first host material also preferably has a structure in which the benzene ring and the naphthalene ring are further linked to each other at any other site than the single bond by the cross-linking structure including a double bond.

Assuming that the benzene ring and the naphthalene ring in the benzene-naphthalene linking structure are further linked to each other at at least one site other than the single bond by cross-linking, for example, a linking structure (fused ring) represented by a formula (X11) below is obtained in a case of the formula (X1), and a linking structure (fused ring) represented by a formula (X31) below is obtained in a case of the formula (X3).

Assuming that the benzene ring and the naphthalene ring in the benzene-naphthalene linking structure are further linked to each other at any other site than the single bond by crosslinking including a double bond, for example, a linking structure (fused ring) represented by a formula (X12) below is obtained in a case of the formula (X1), a linking structure (fused ring) represented by a formula (X21) or formula (X22) below is obtained in a case of the formula (X2), a linking structure (fused ring) represented by a formula (X41) below is obtained in a case of the formula (X4), and a linking structure (fused ring) represented by a formula (X51) below is obtained in a case of the formula (X5).

Assuming that the benzene ring and the naphthalene ring in the benzene-naphthalene linking structure are further linked to each other at at least one site other than the single bond by cross-linking including a hetero atom (e.g., an oxygen atom), for example, a linking structure (fused ring) represented by a formula (X13) below is obtained in a case of the formula (X1).

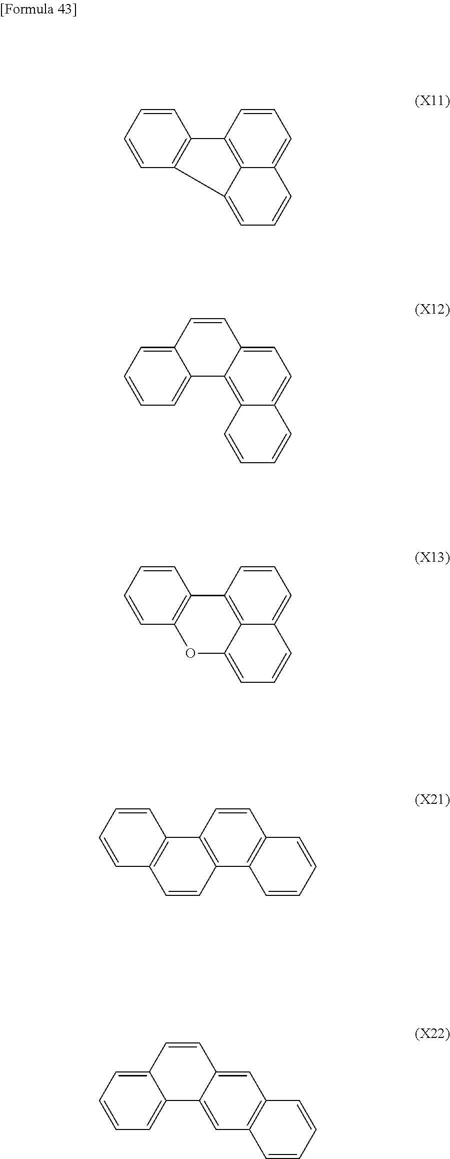

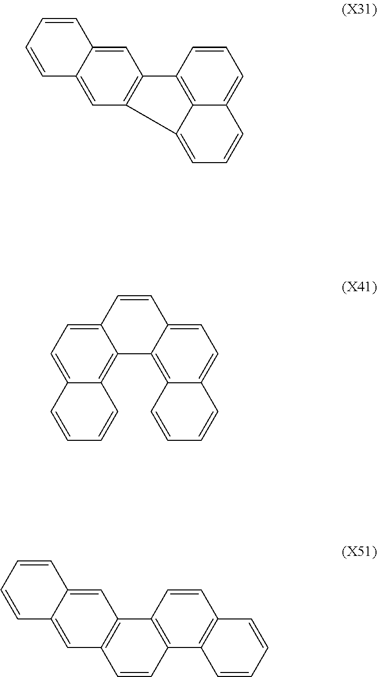

In the organic EL device according to the exemplary embodiment, it is also preferable that: the first host material has, in a molecule, a biphenyl structure in which a first benzene ring and a second benzene ring are linked to each other with a single bond; and the first benzene ring and the second benzene ring in the biphenyl structure are further linked to each other by cross-linking at at least one site other than the single bond.

In the organic EL device according to the exemplary embodiment, it is also preferable that the first benzene ring and the second benzene ring in the biphenyl structure are further linked to each other by the cross-linking at one site other than the single bond. Since the first host material has the biphenyl structure including such cross-linking, it can be expected to inhibit the deterioration in the chromaticity of the organic EL device.

In the organic EL device according to the exemplary embodiment, the cross-linking also preferably includes a double bond.

In the organic EL device according to the exemplary embodiment, it is also preferable that the cross-linking does not include a double bond.

It is also preferable that the first benzene ring and the second benzene ring in the biphenyl structure are further linked to each other by the cross-linking in two sites other than the single bond.

In the organic EL device according to the exemplary embodiment, it is also preferable that the first benzene ring and the second benzene ring in the biphenyl structure are further linked to each other by the cross-linking in two sites other than the single bond and the cross-linking includes no double bond. Since the first host material has the biphenyl structure including such cross-linking, it can be expected to inhibit the deterioration in the chromaticity of the organic EL device.

For example, assuming that the first benzene ring and the second benzene ring in the biphenyl structure represented by a formula (BP1) below are further linked to each other by cross-linking at at least one site other than the single bond, the biphenyl structure is exemplified by linking structures (fused rings) represented by formulae (BP11) to (BP15) below.

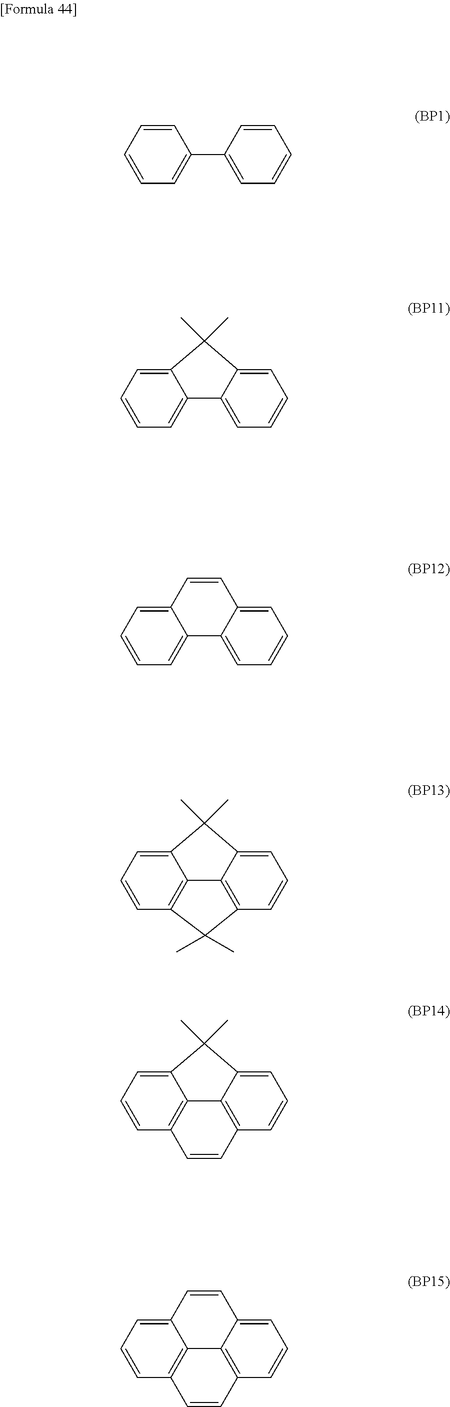

The formula (BP11) represents a linking structure in which the first benzene ring and the second benzene ring are linked to each other at one site other than the single bond by cross-linking including no double bond.

The formula (BP12) represents a linking structure in which the first benzene ring and the second benzene ring are linked to each other at one site other than the single bond by cross-linking including a double bond.

The formula (BP13) represents a linking structure in which the first benzene ring and the second benzene ring are linked to each other at two sites other than the single bond by cross-linking including no double bond.

The formula (BP14) represents a linking structure in which the first benzene ring and the second benzene ring are linked to each other at one of two sites other than the single bond by cross-linking including no double bond while being linked to each other at the other of the two sites other than the single bond by cross-linking including a double bond.

The formula (BP15) represents a linking structure in which the first benzene ring and the second benzene ring are linked to each other at two sites other than the single bond by cross-linking including double bonds.

In the first compound and the second compound, it is preferable that all groups described as “substituted or unsubstituted” groups are “unsubstituted” groups.

### Manufacturing Method of First Compound

The first compound can be manufactured by a known method. The first compound can also be manufactured based on a known method through a known alternative reaction using a known material(s) tailored for the target compound.

### Specific Examples of First Compound

Specific examples of the first compound include the following compounds. It should however be noted that the invention is not limited by the specific examples of the first compound.

In the specific examples of the compound herein, D represents a deuterium atom, Me represents a methyl group, and tBu represents a tert-butyl group.

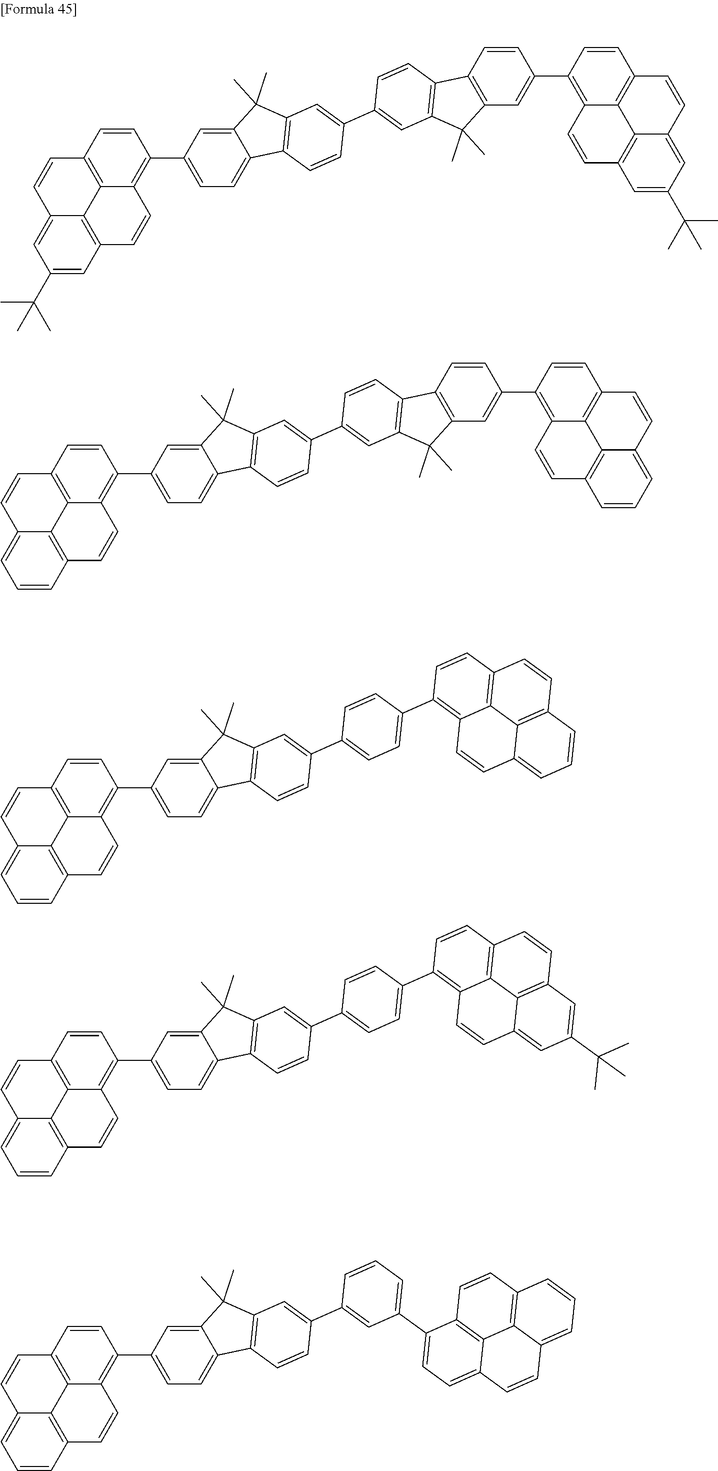

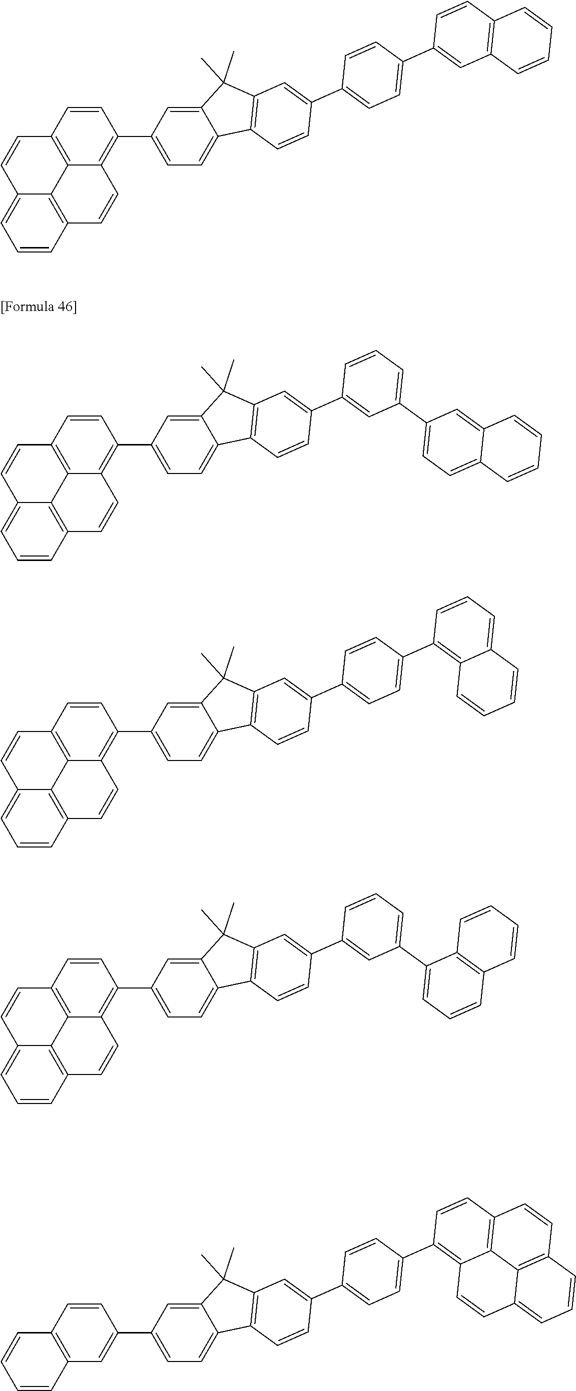

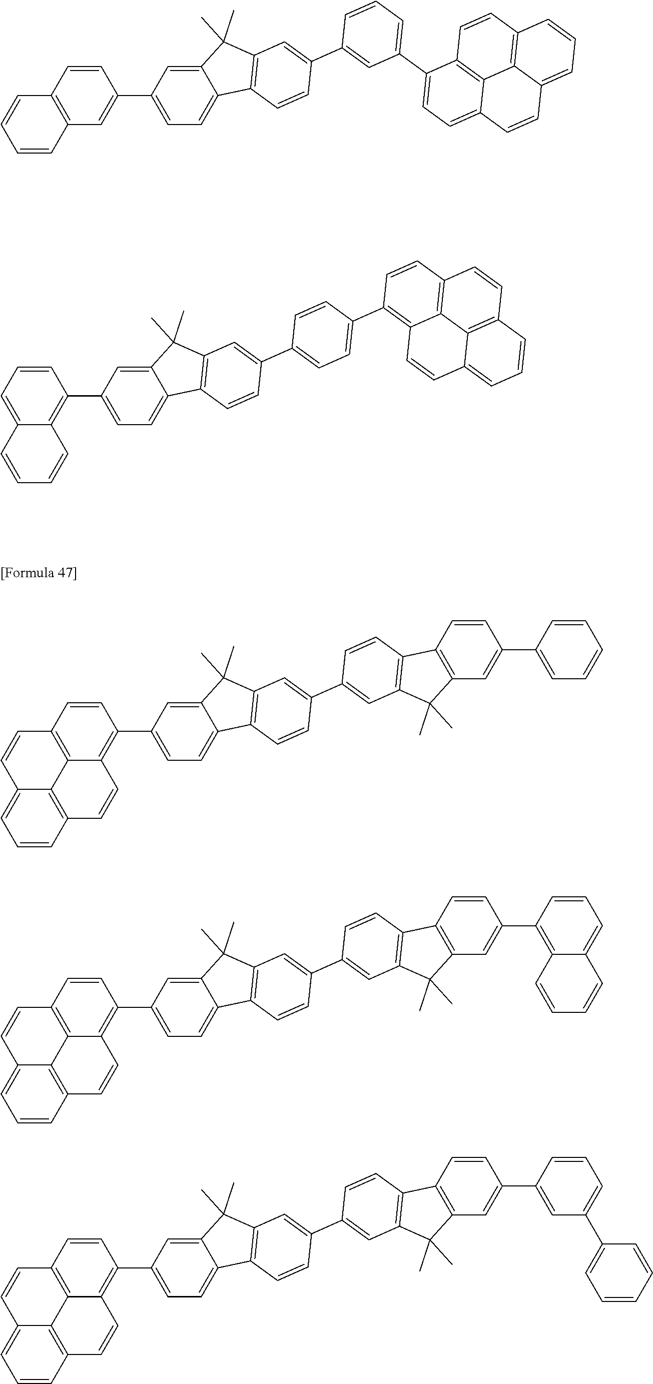

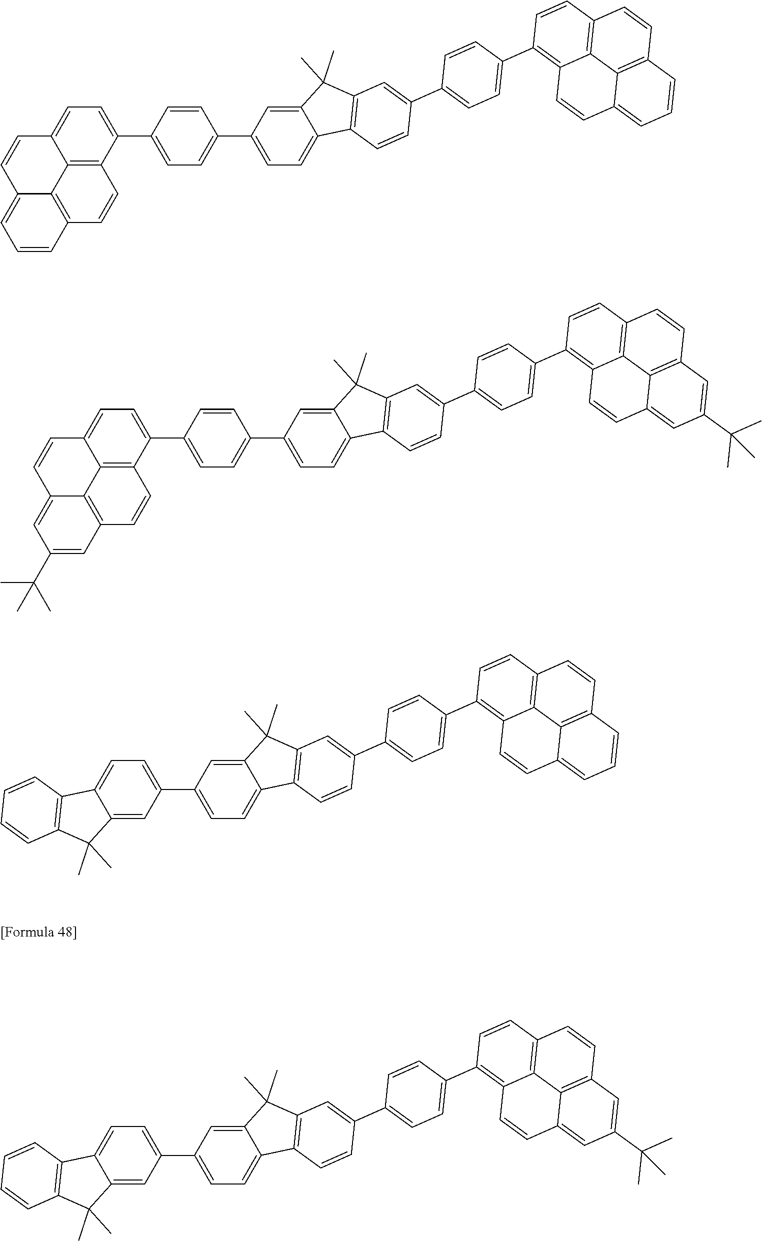

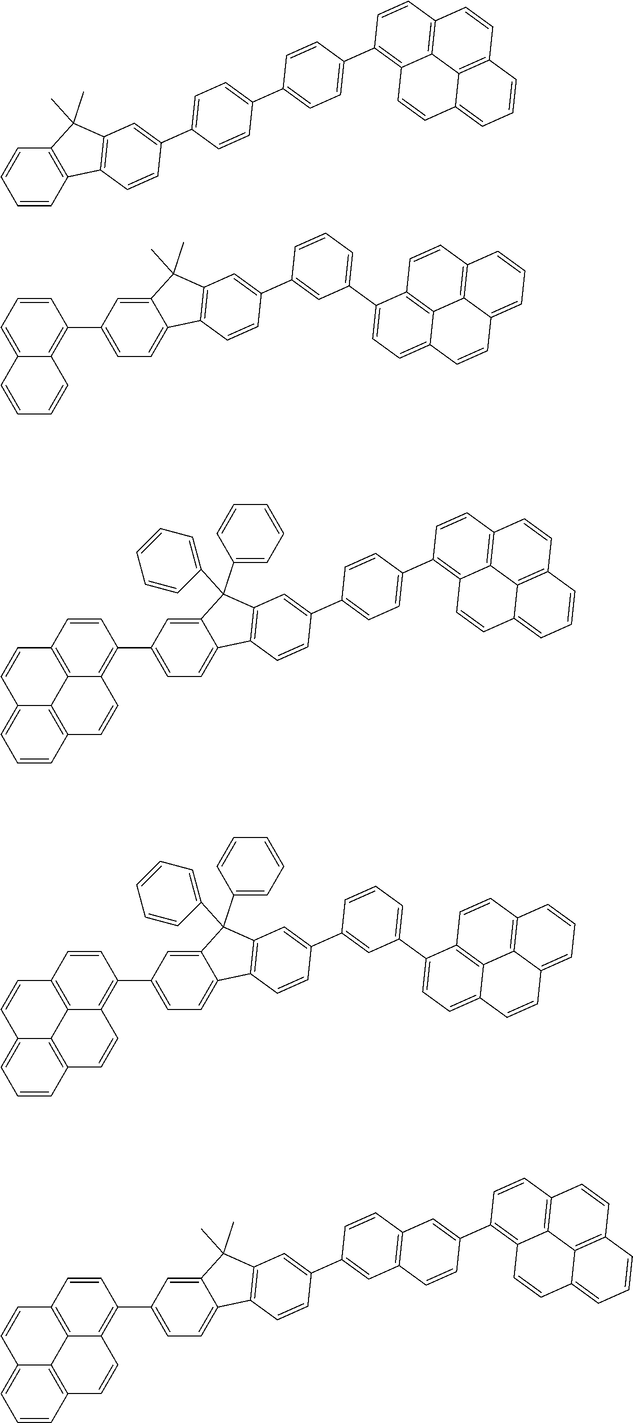

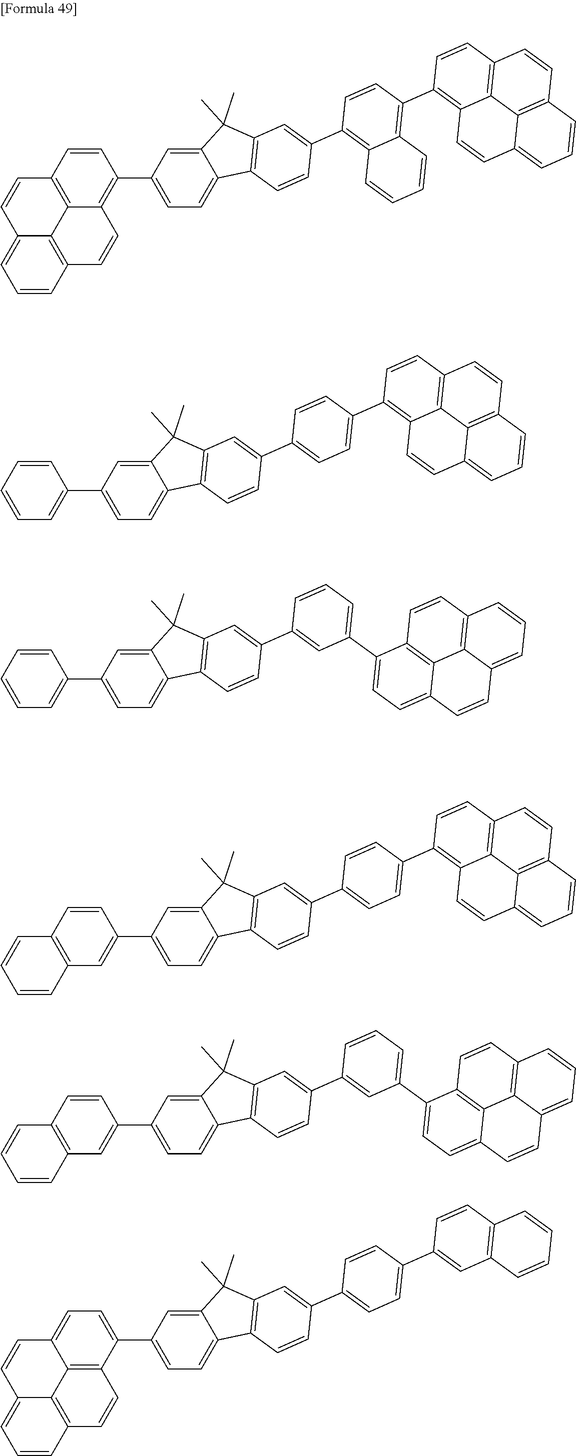

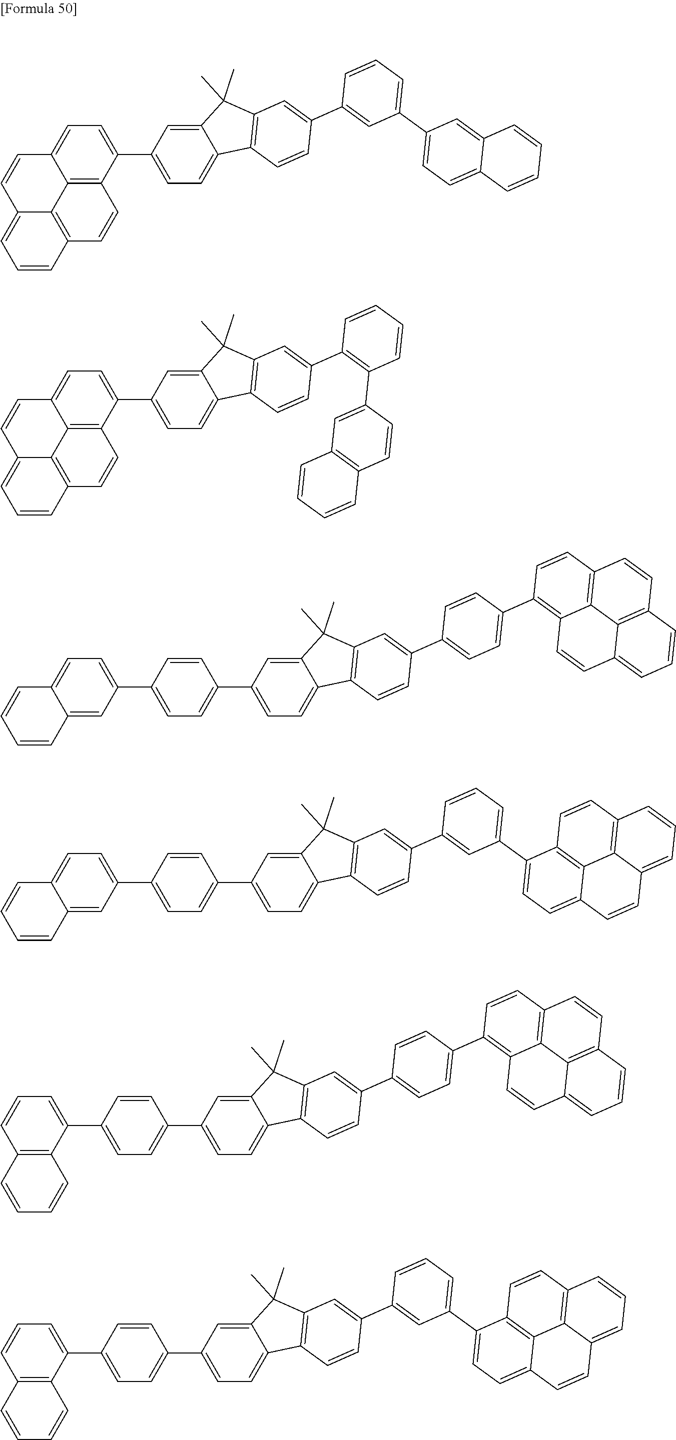

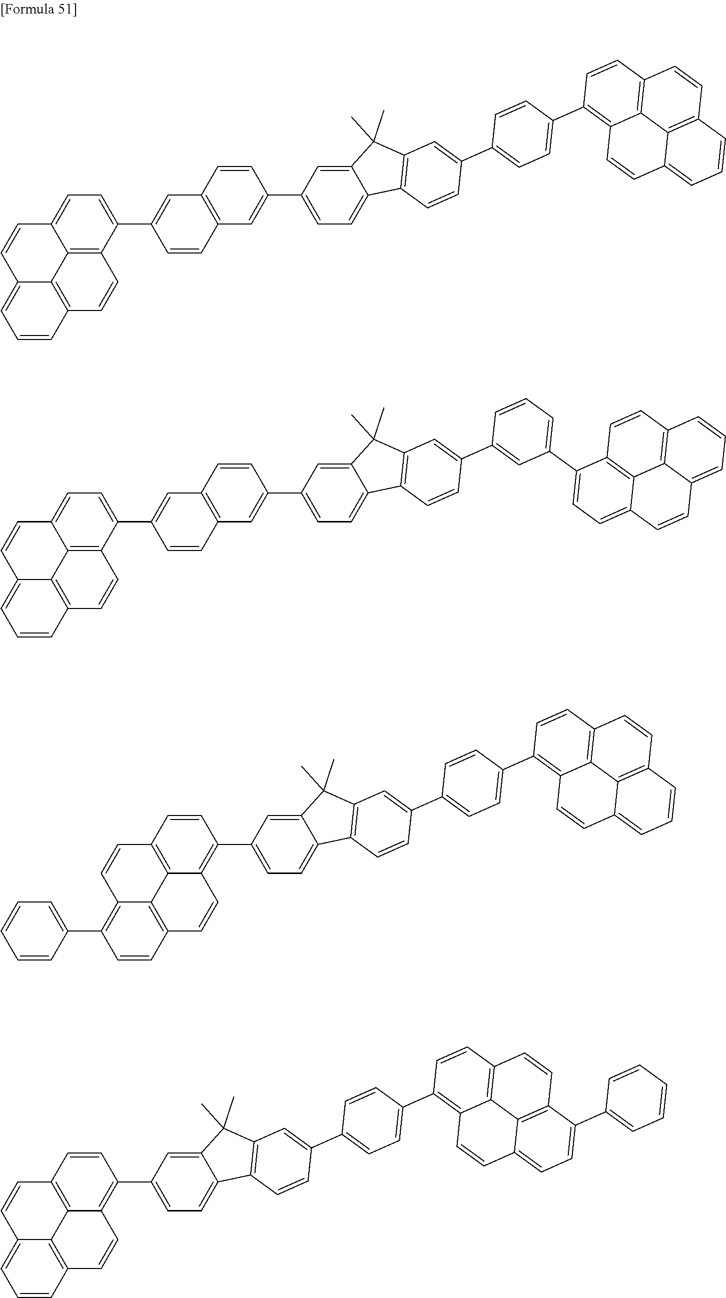

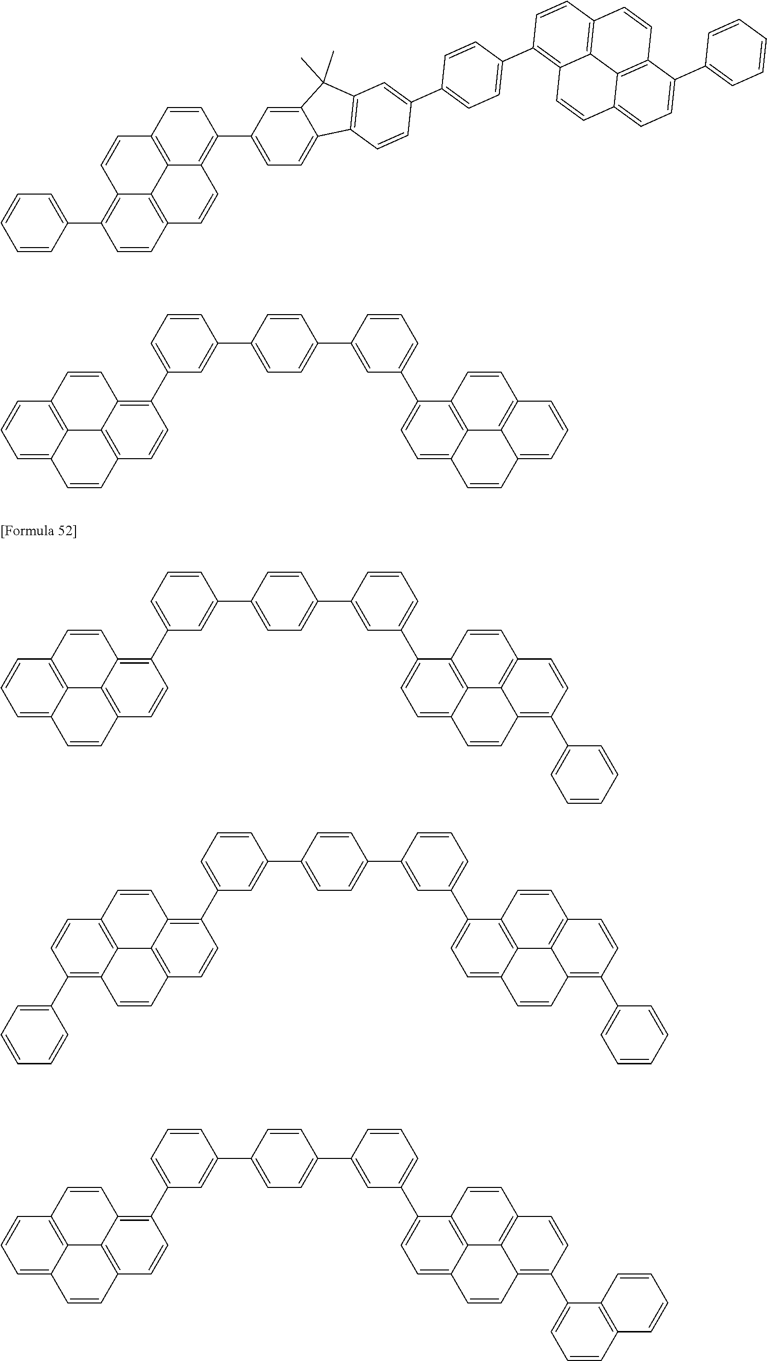

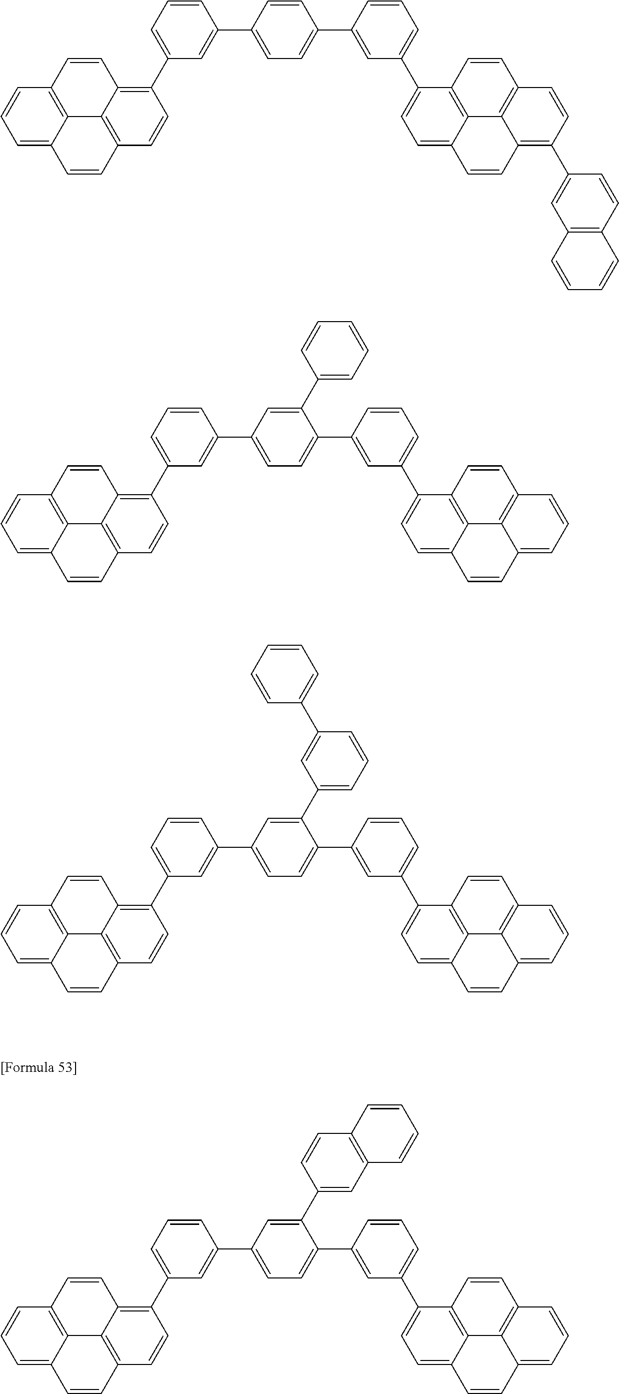

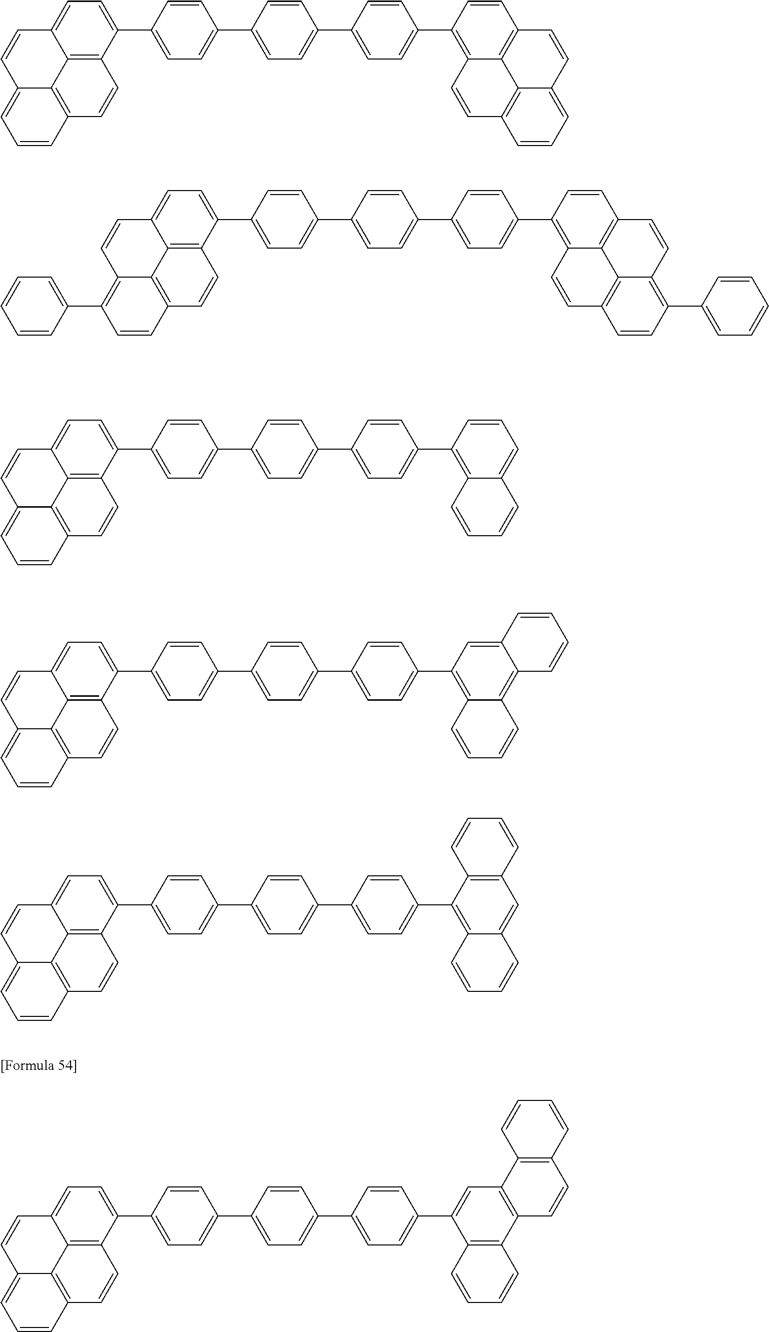

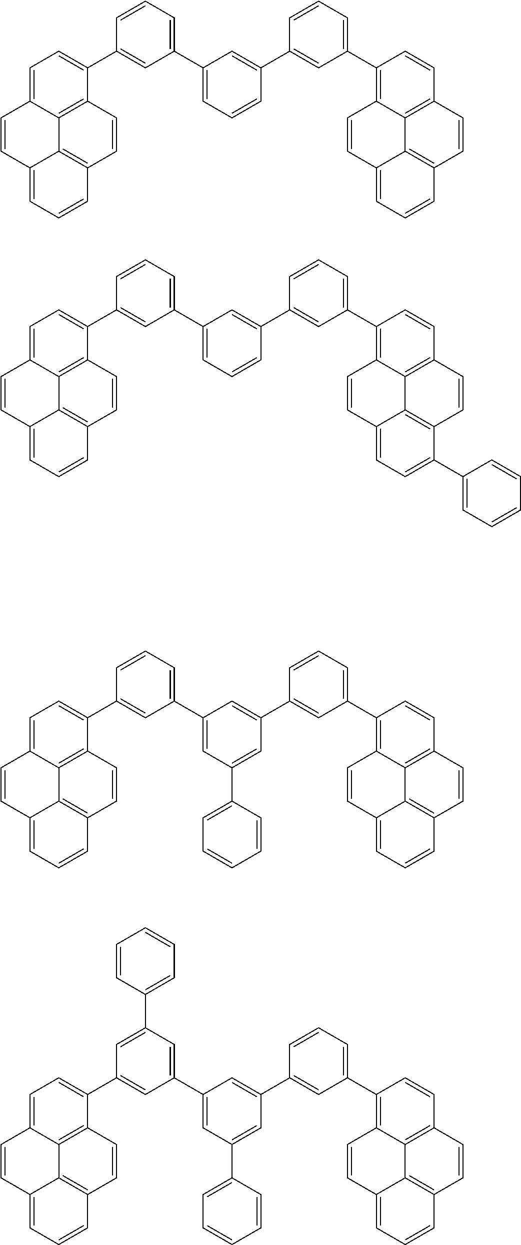

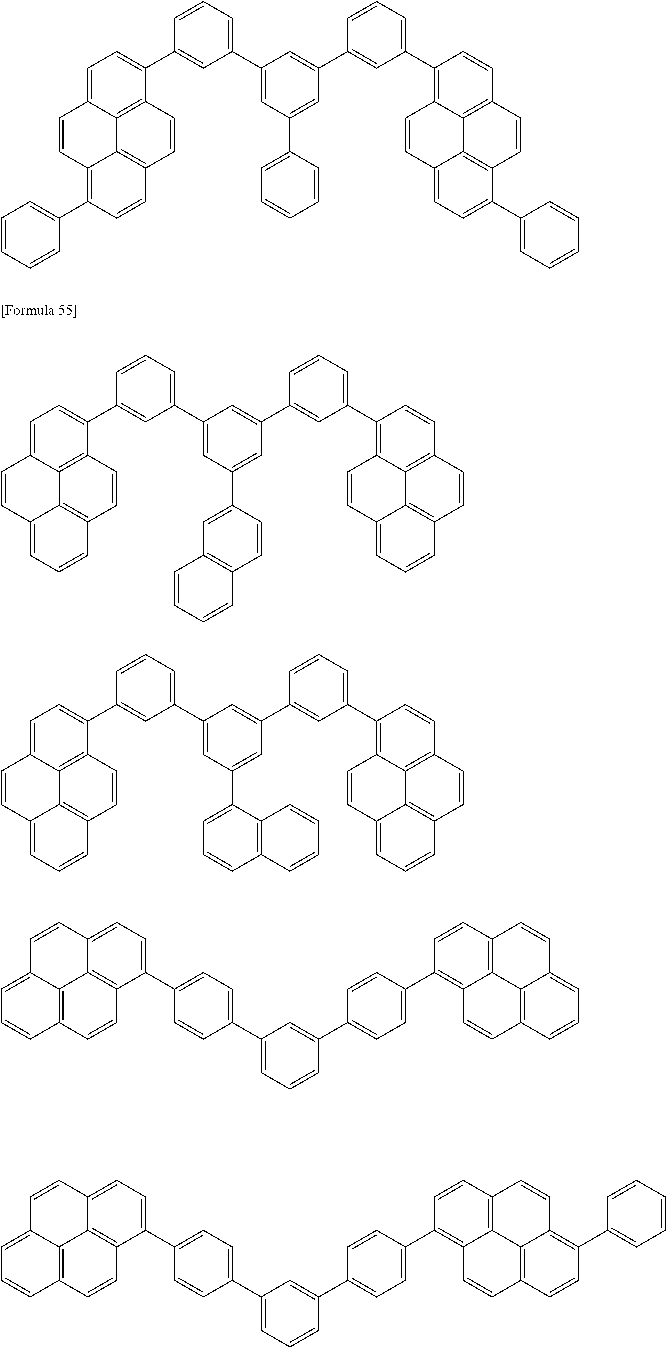

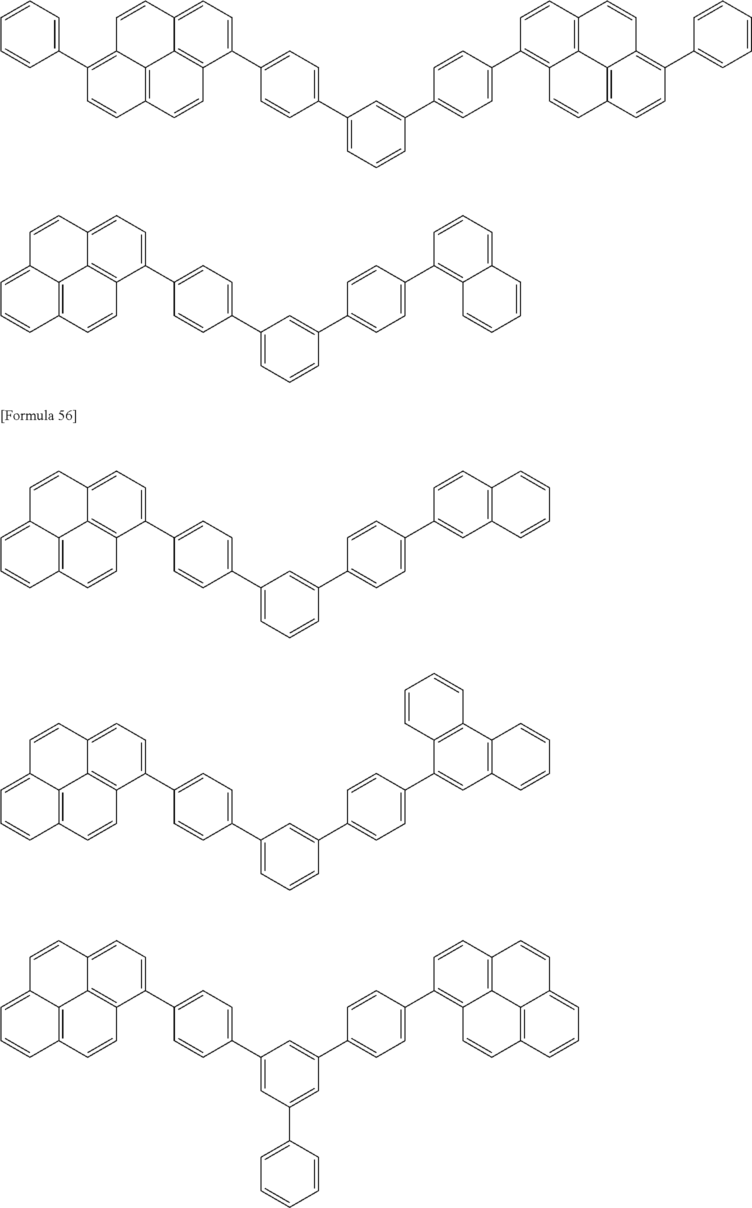

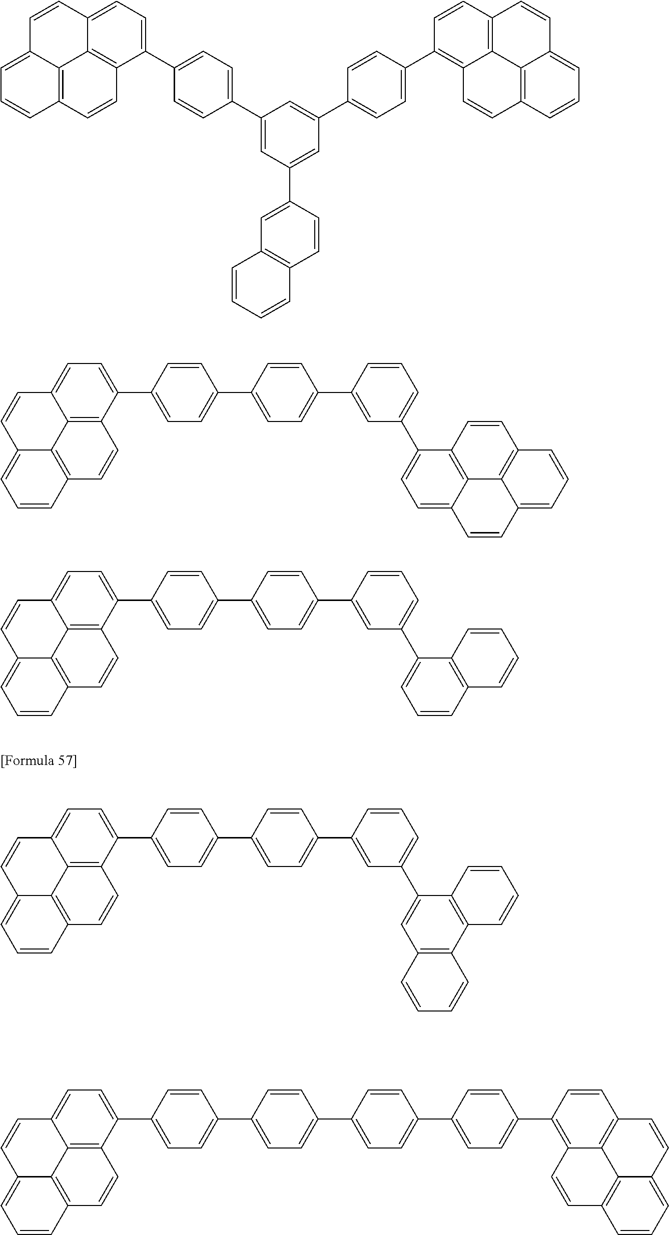

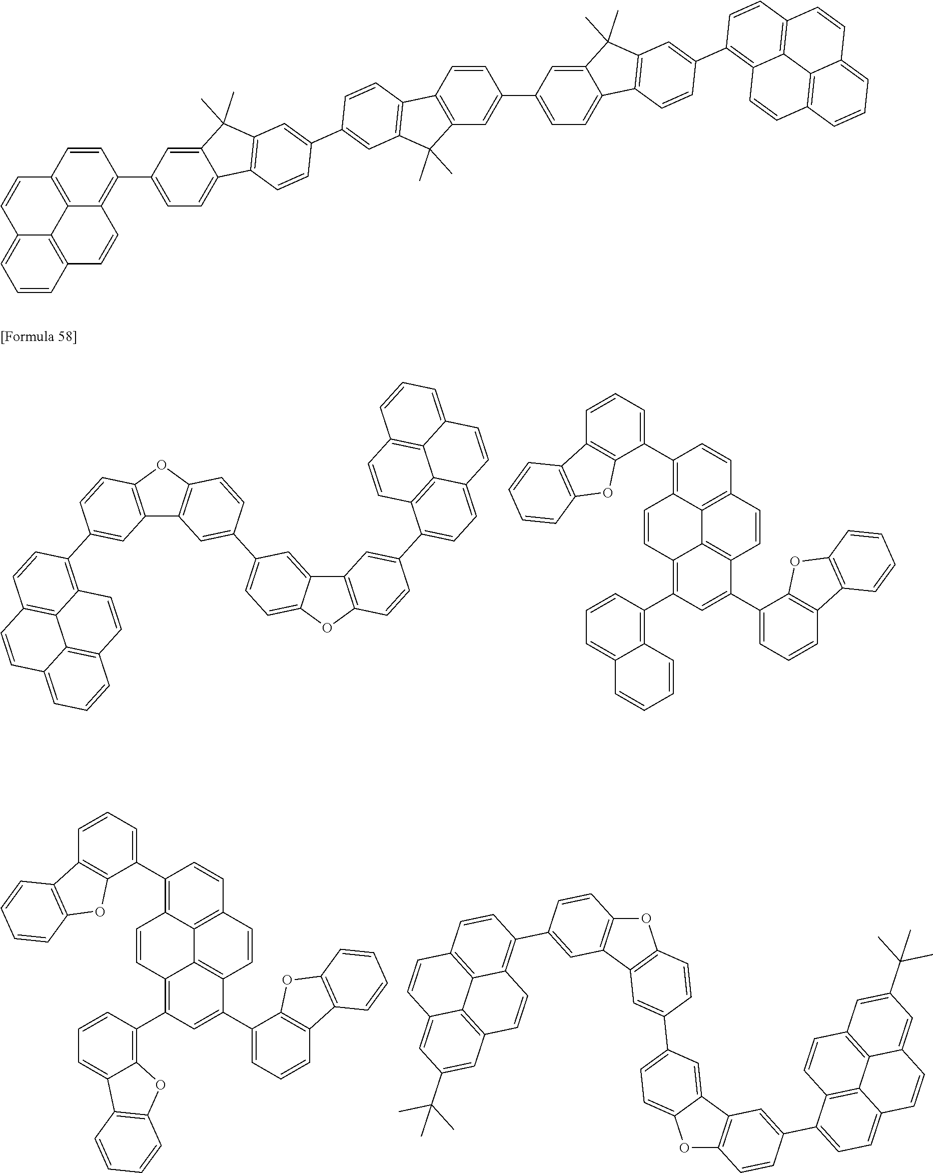

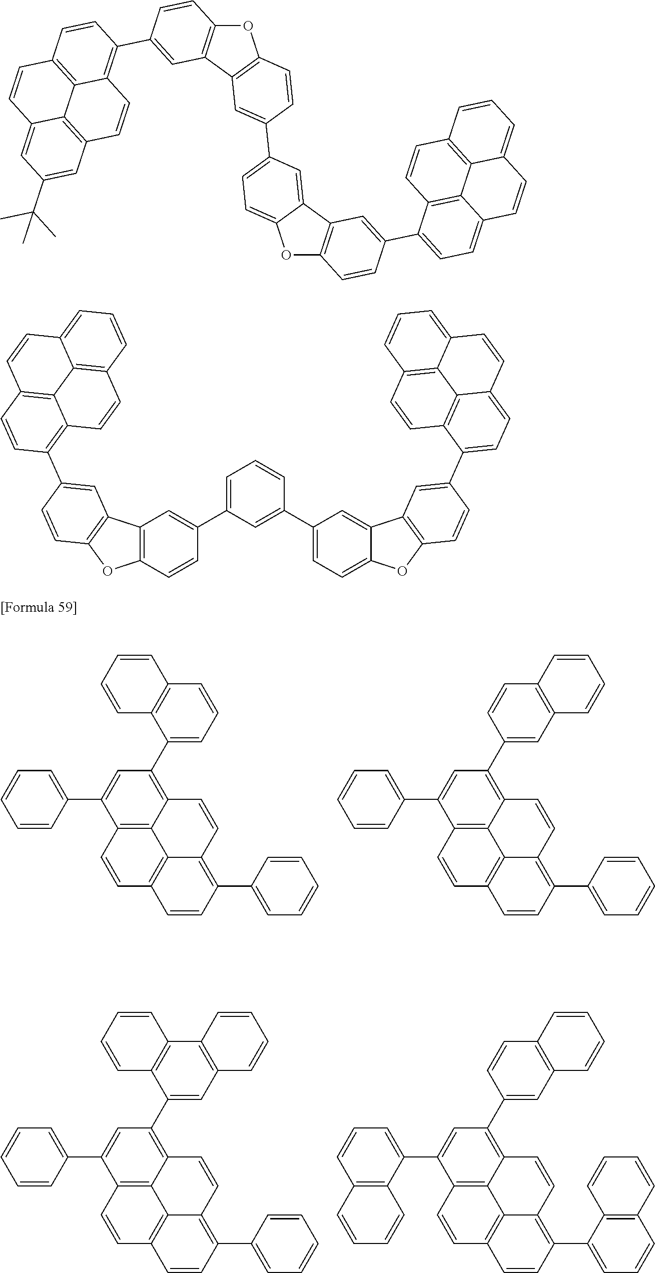

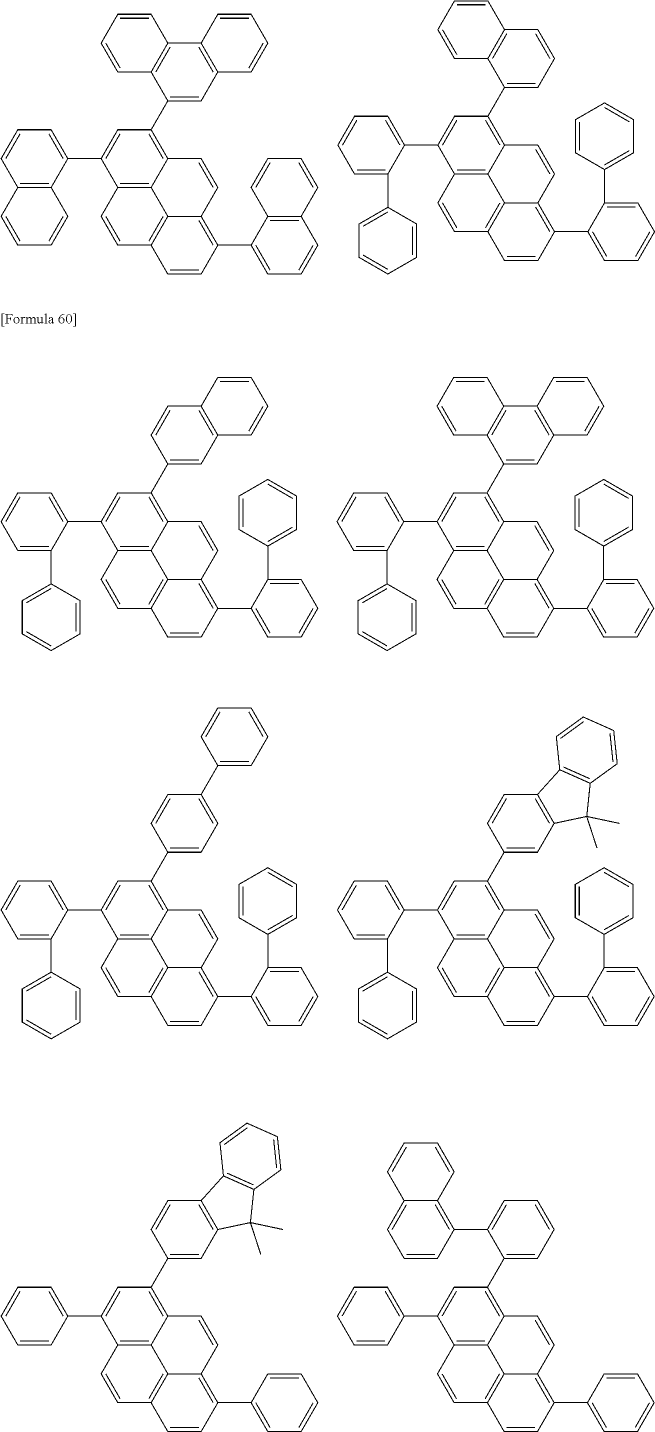

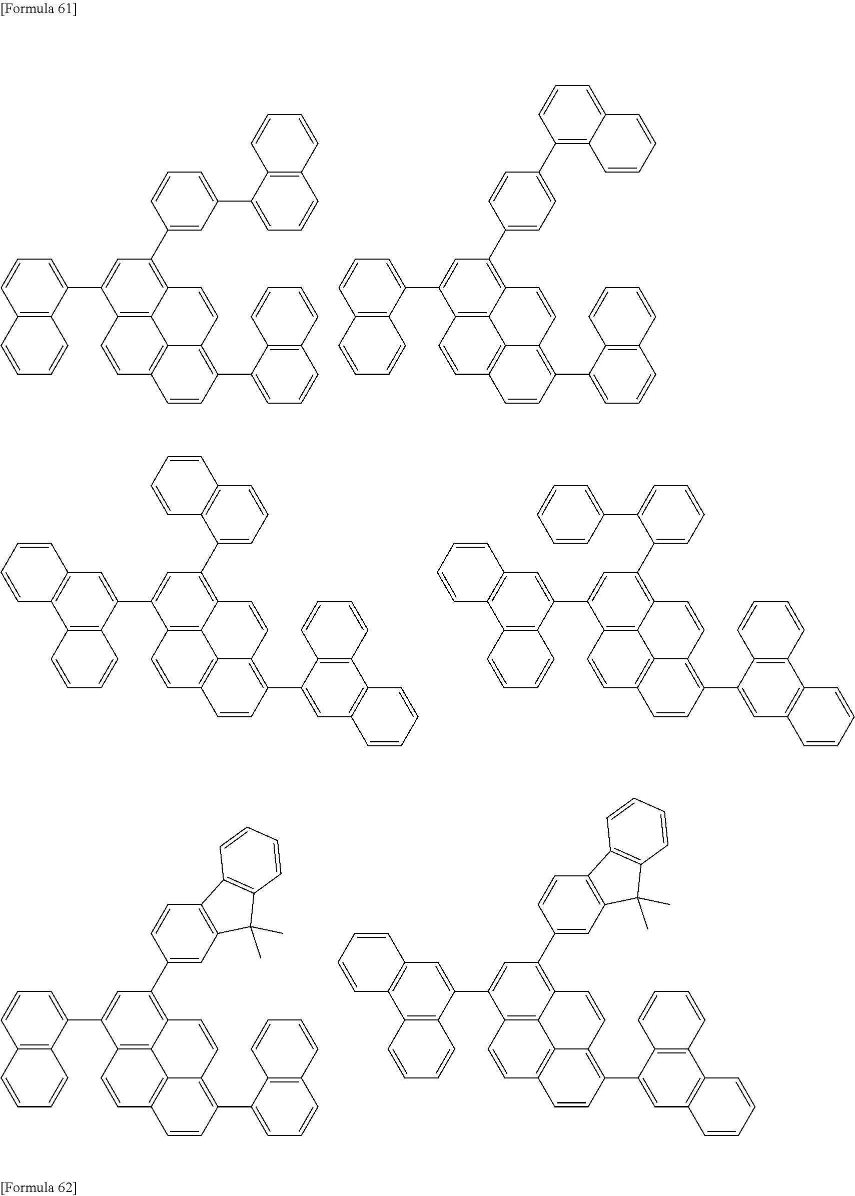

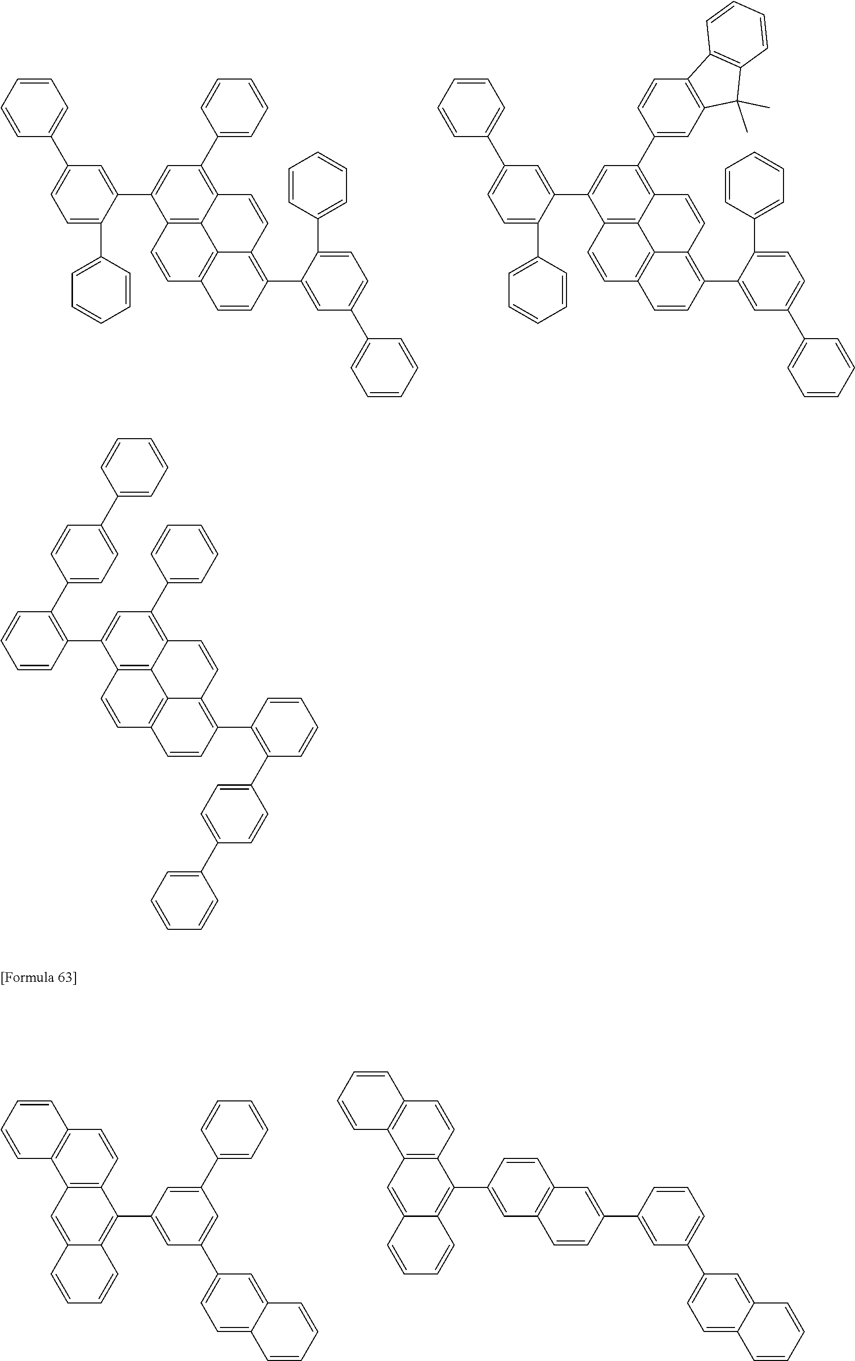

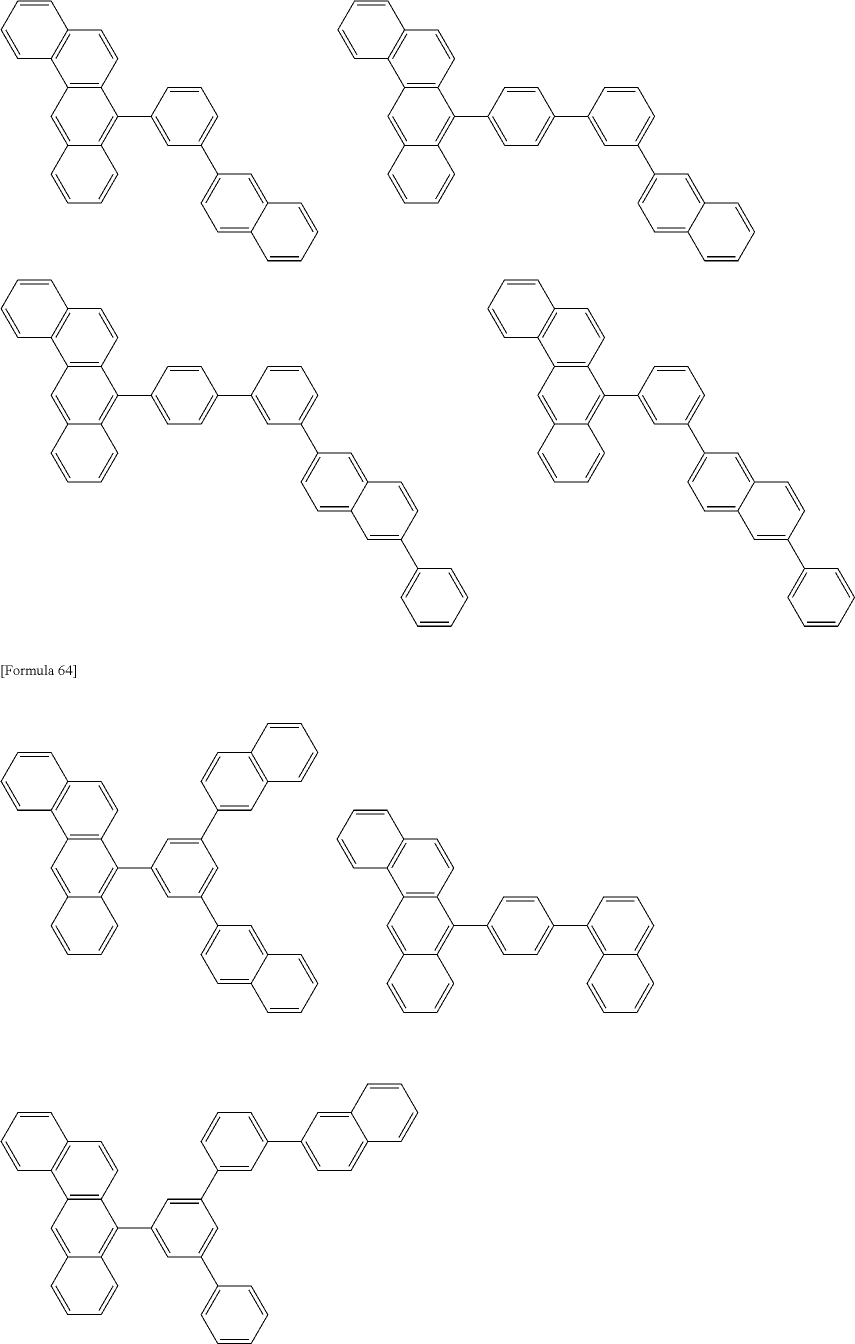

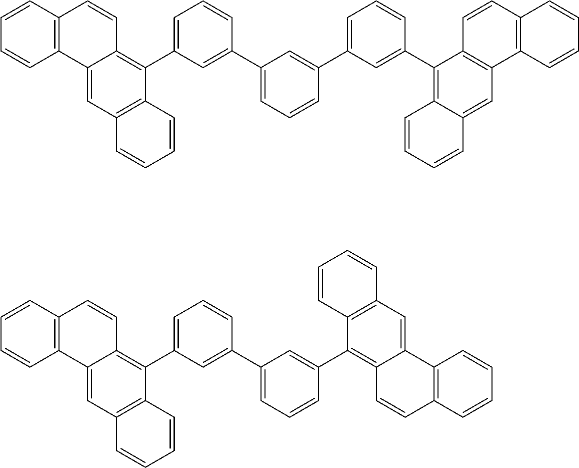

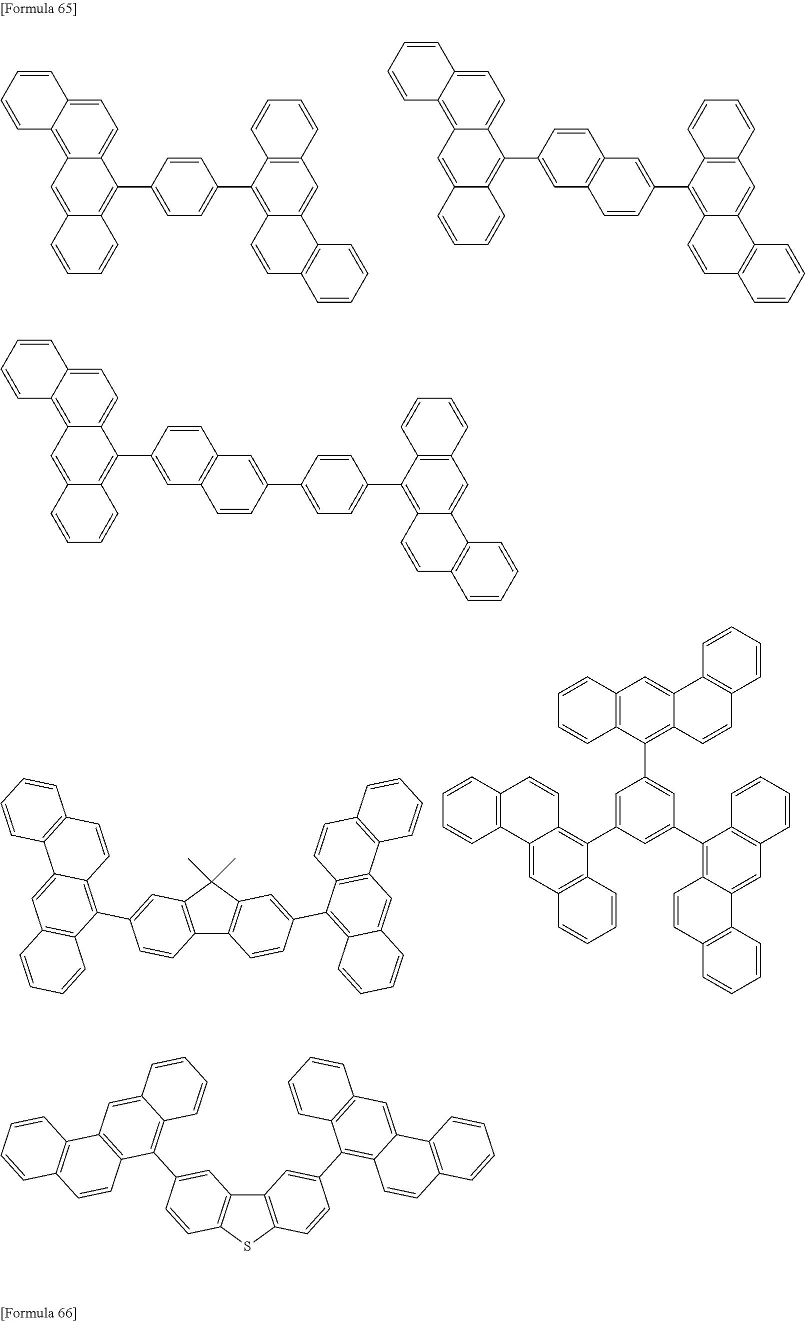

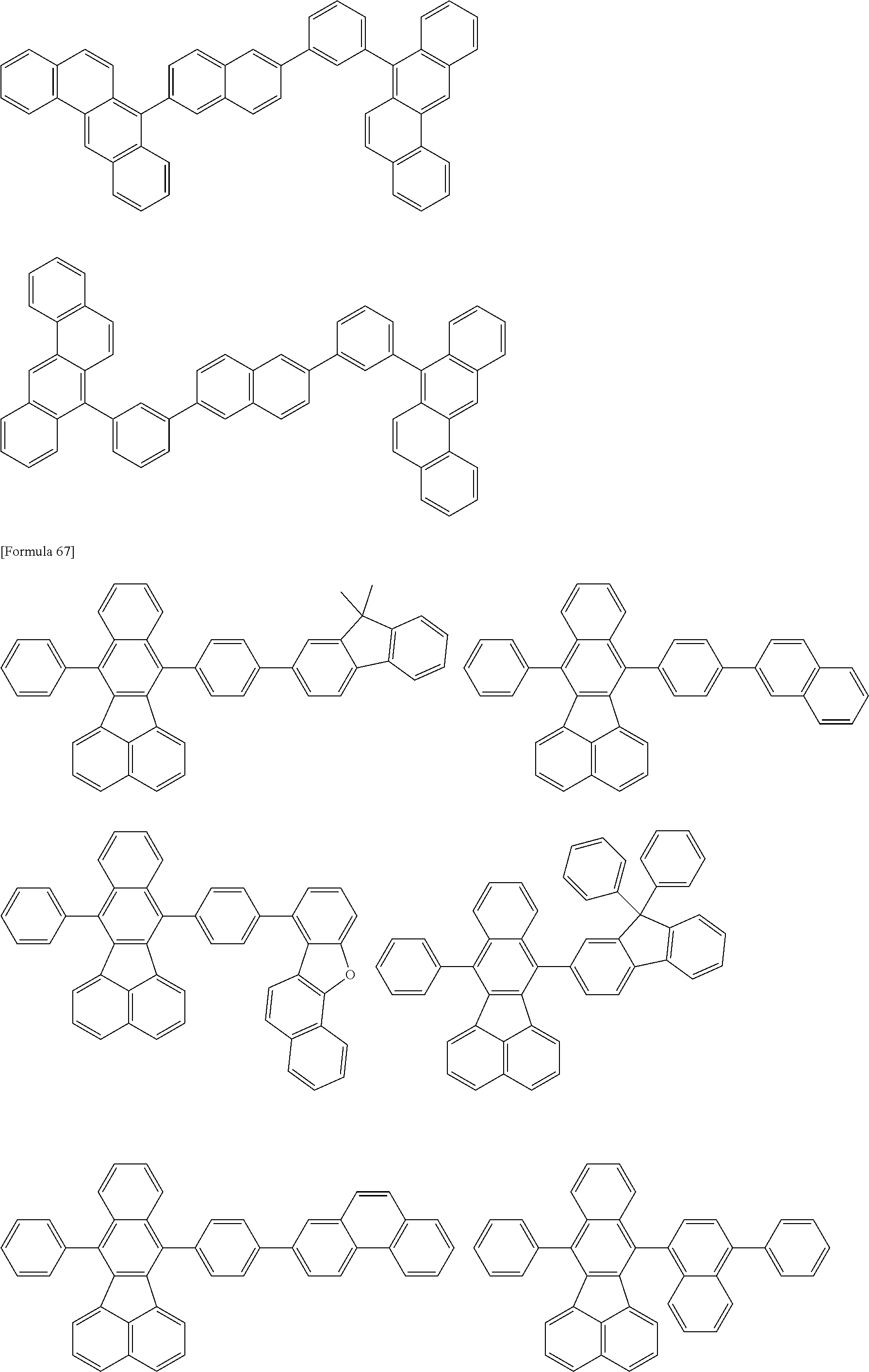

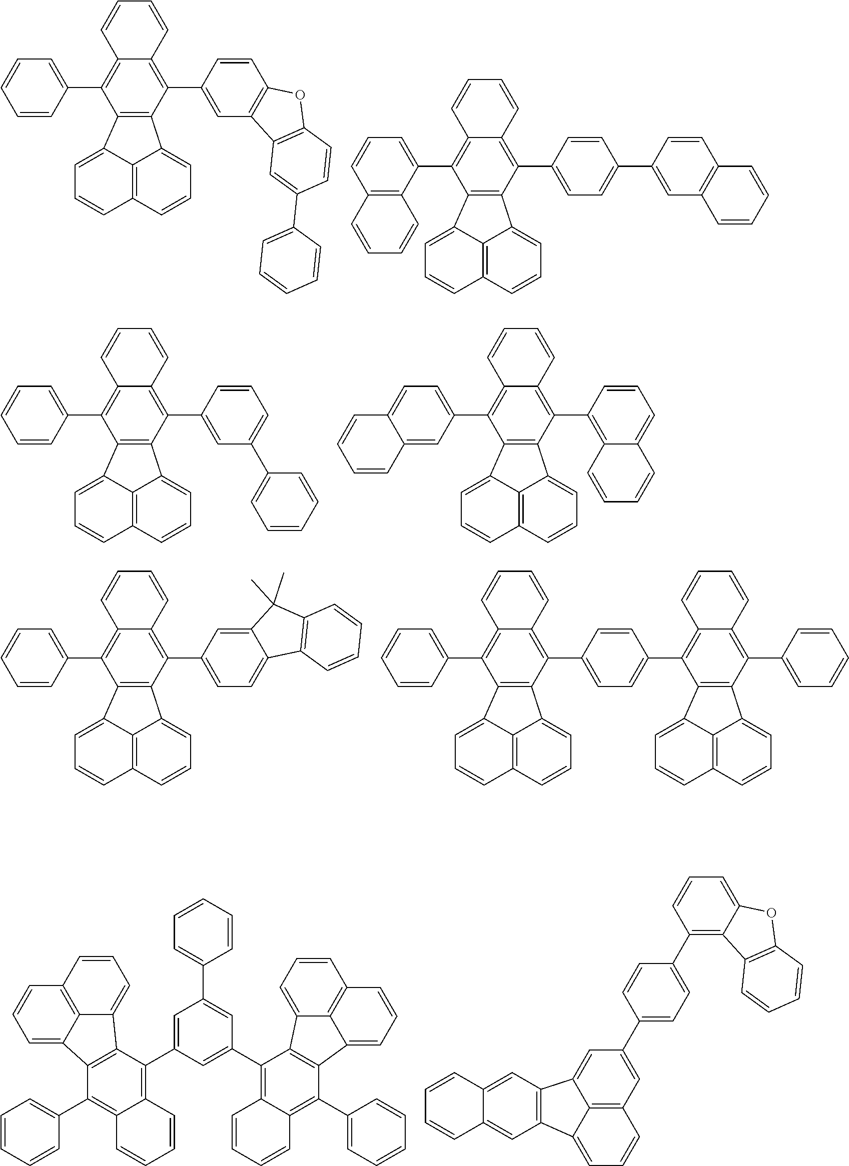

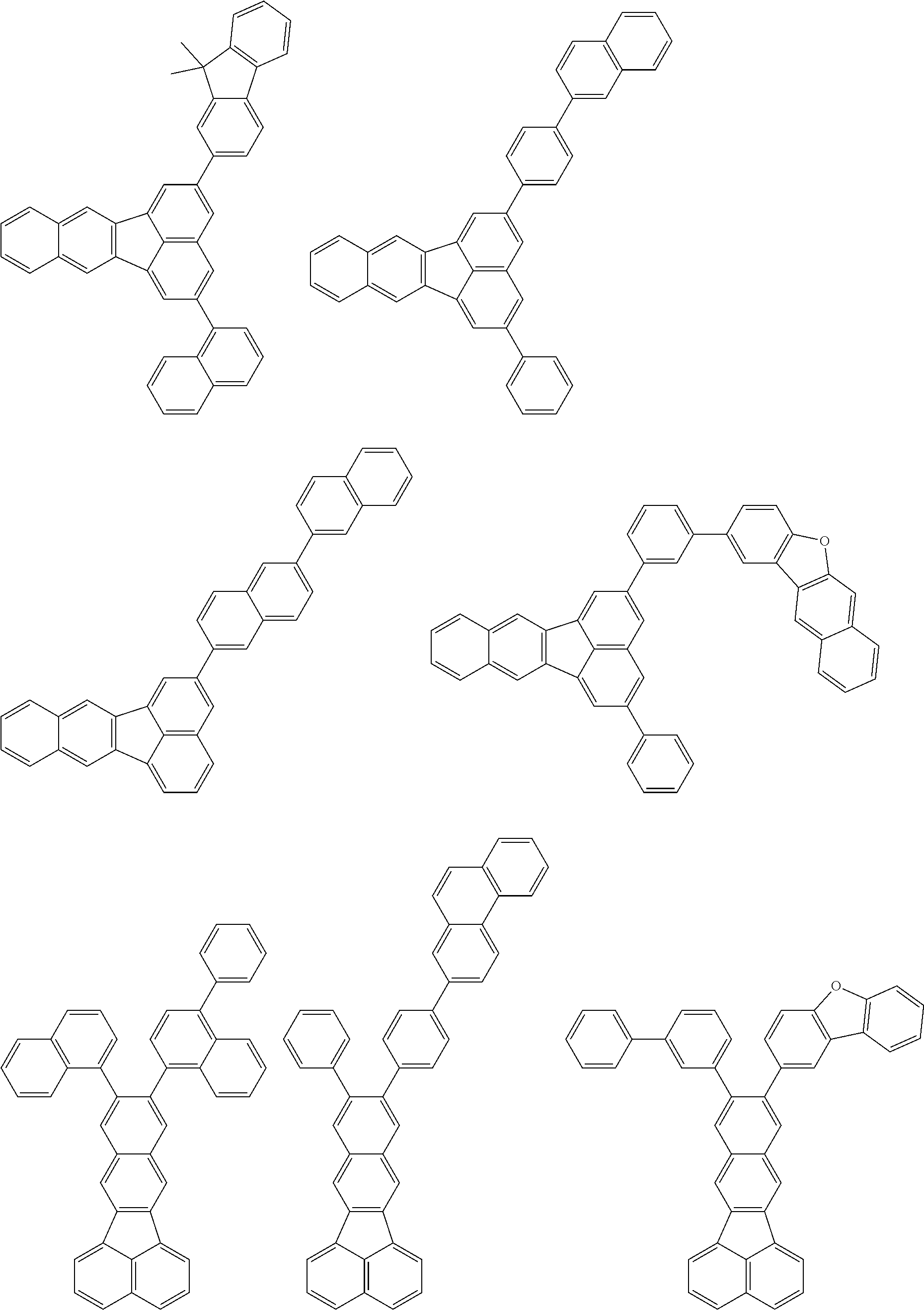

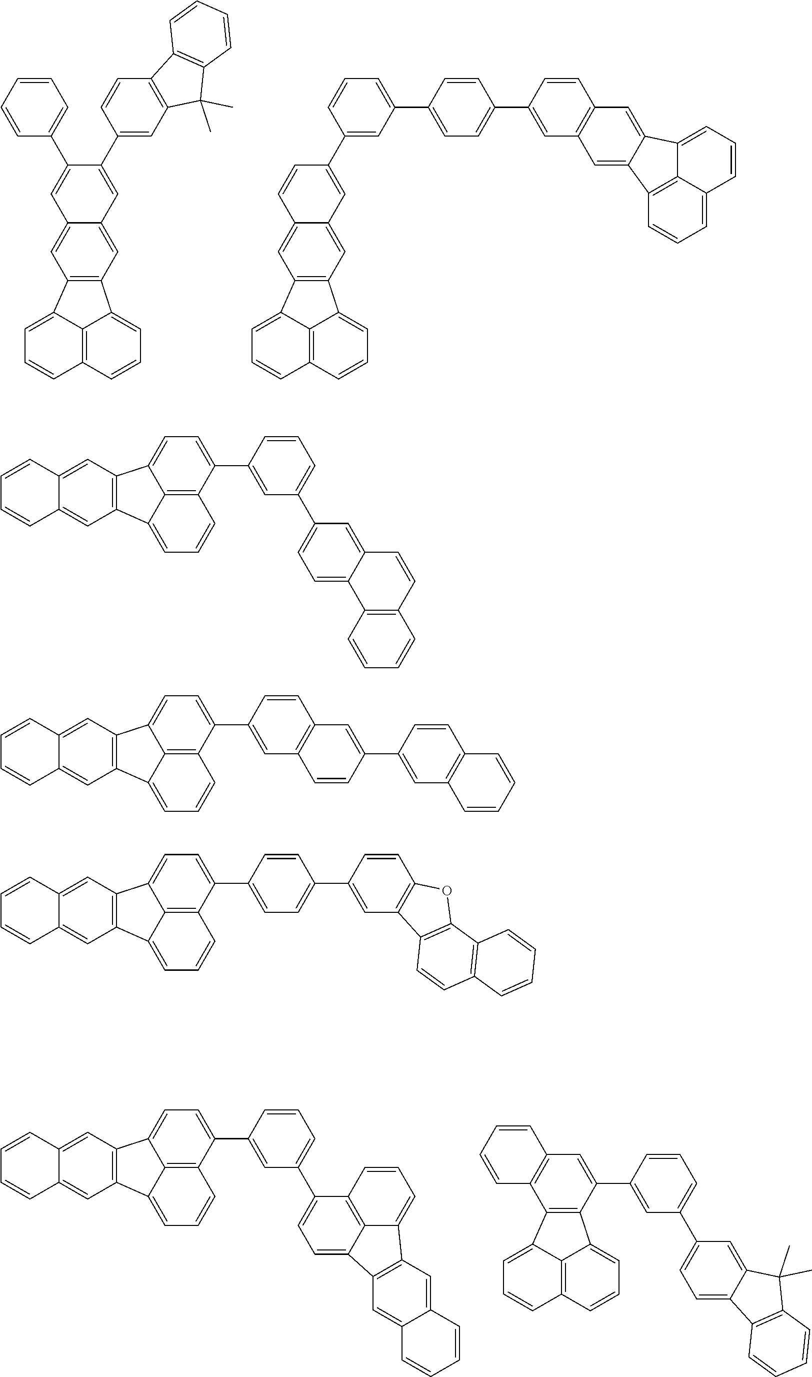

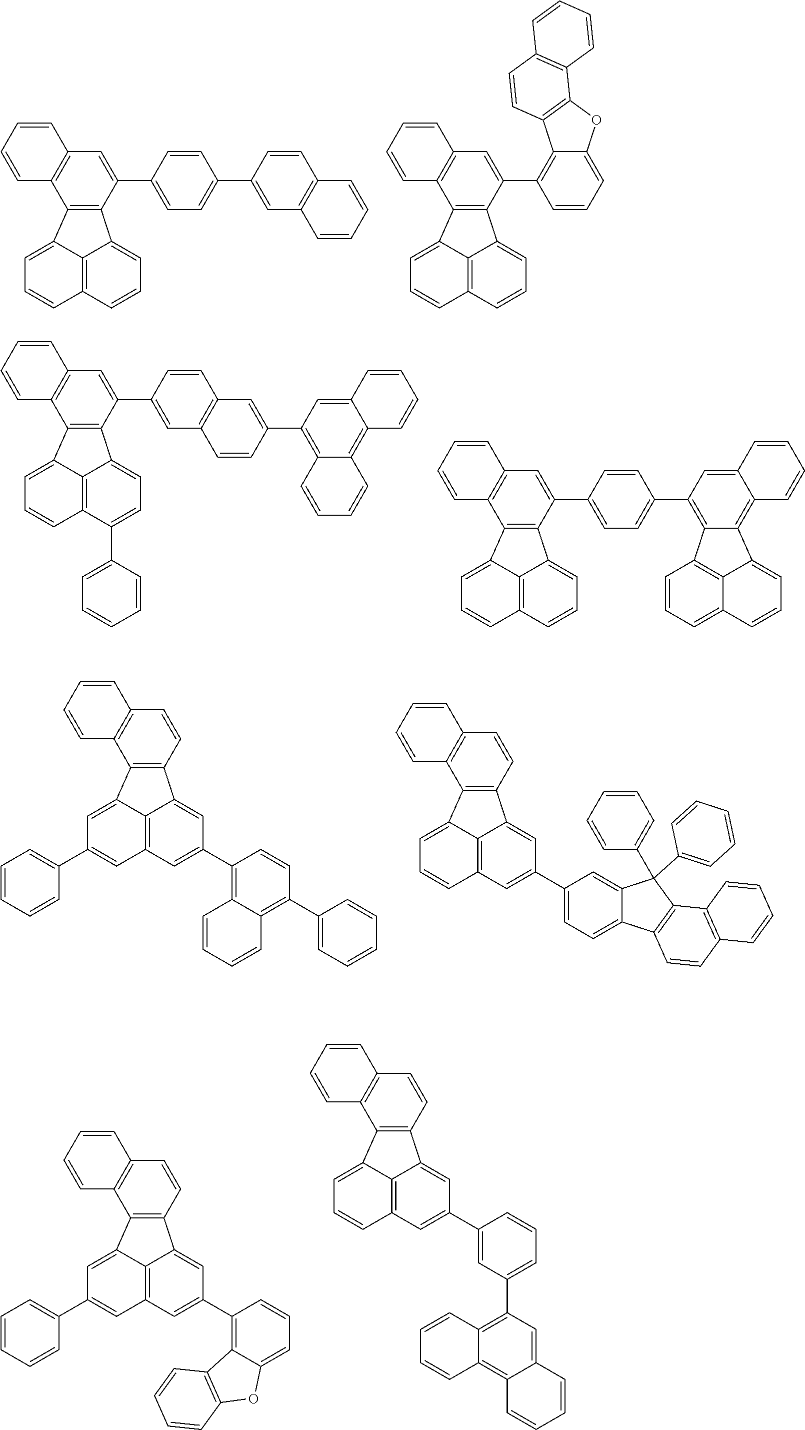

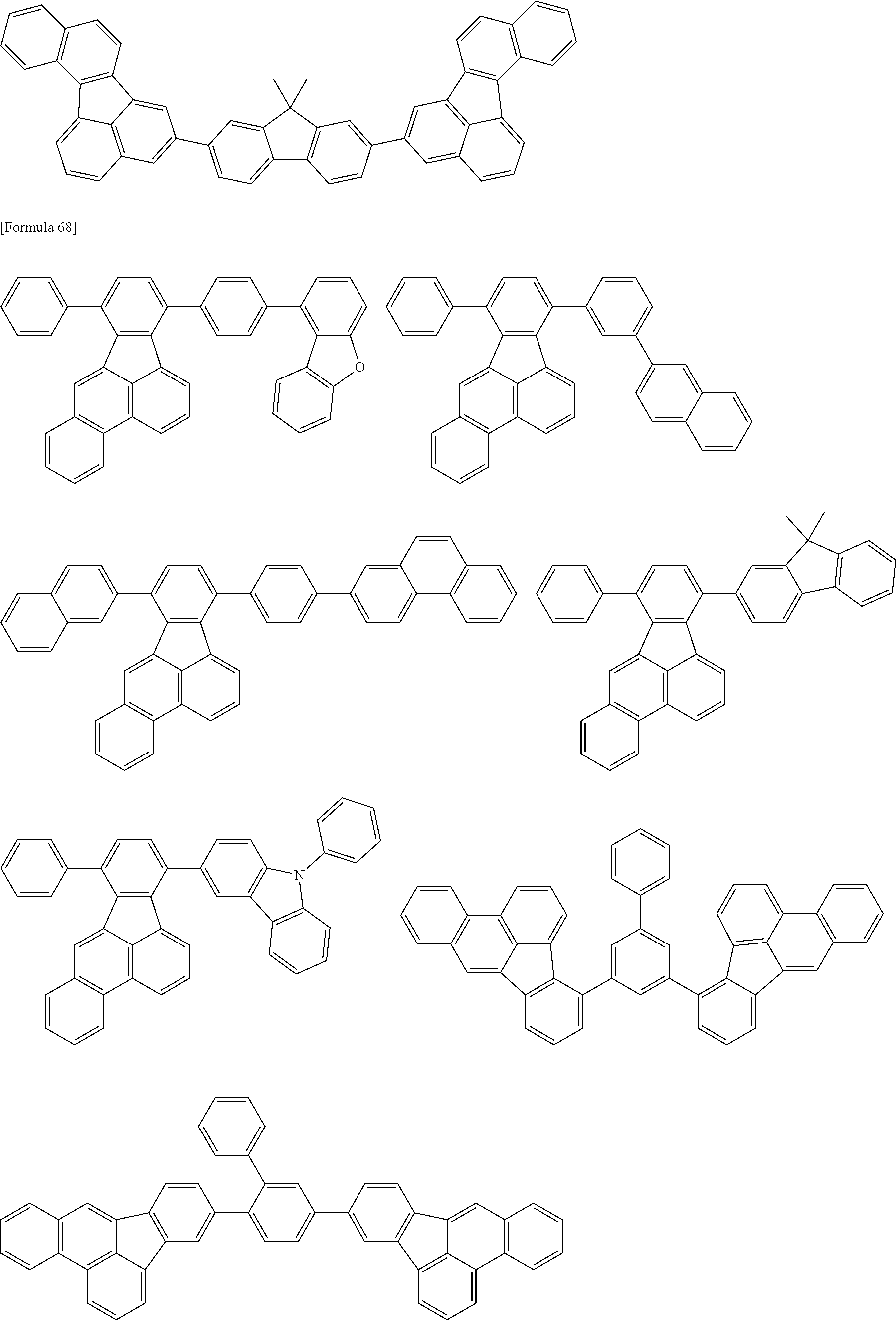

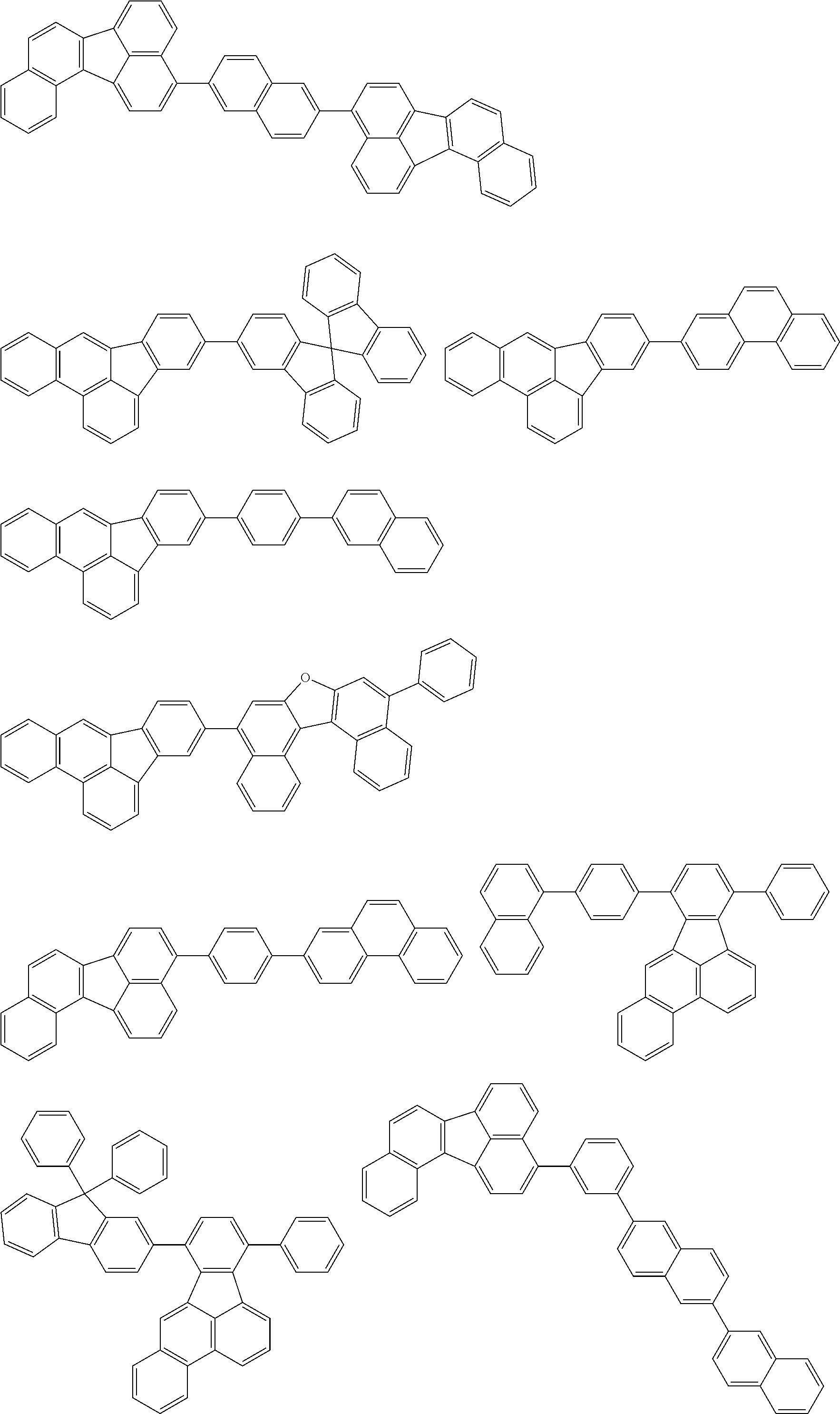

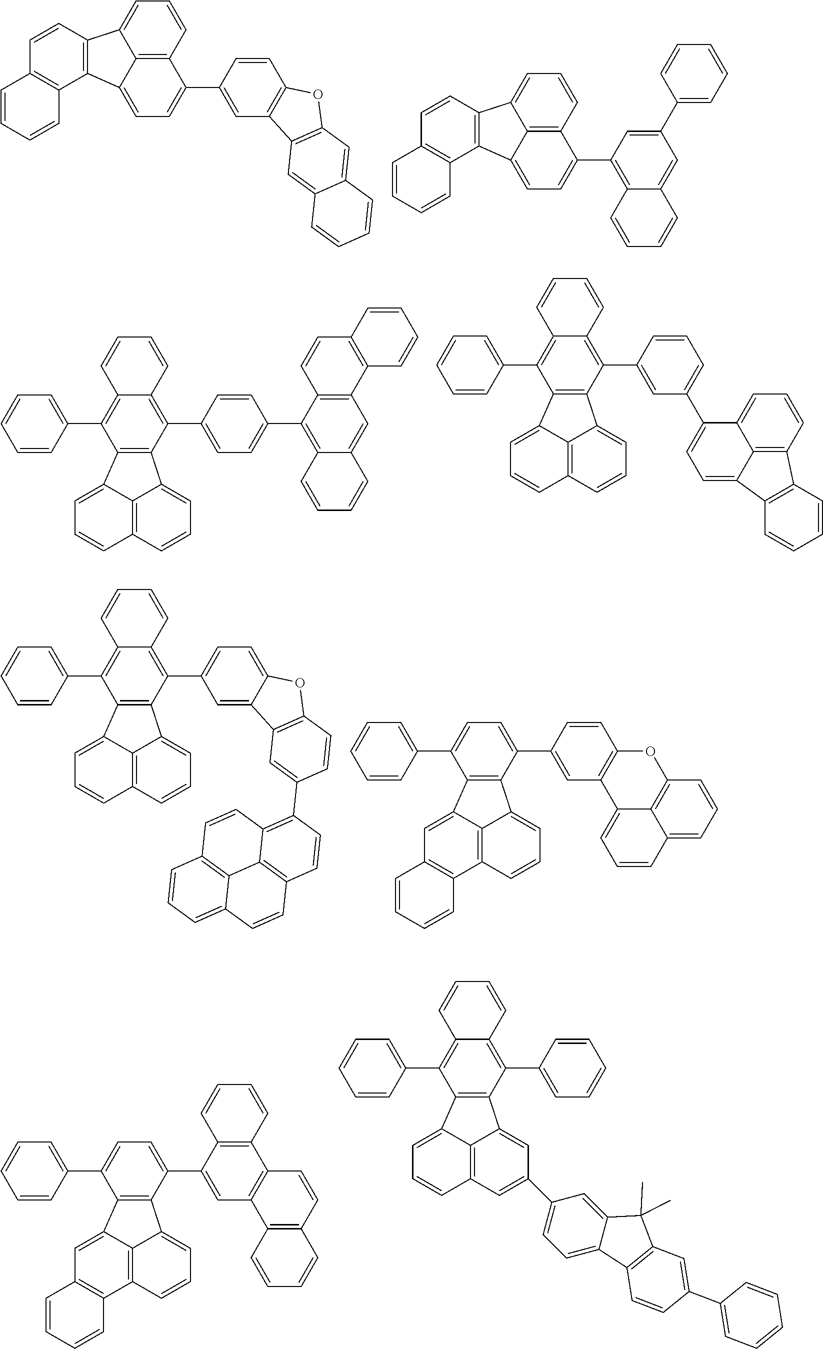

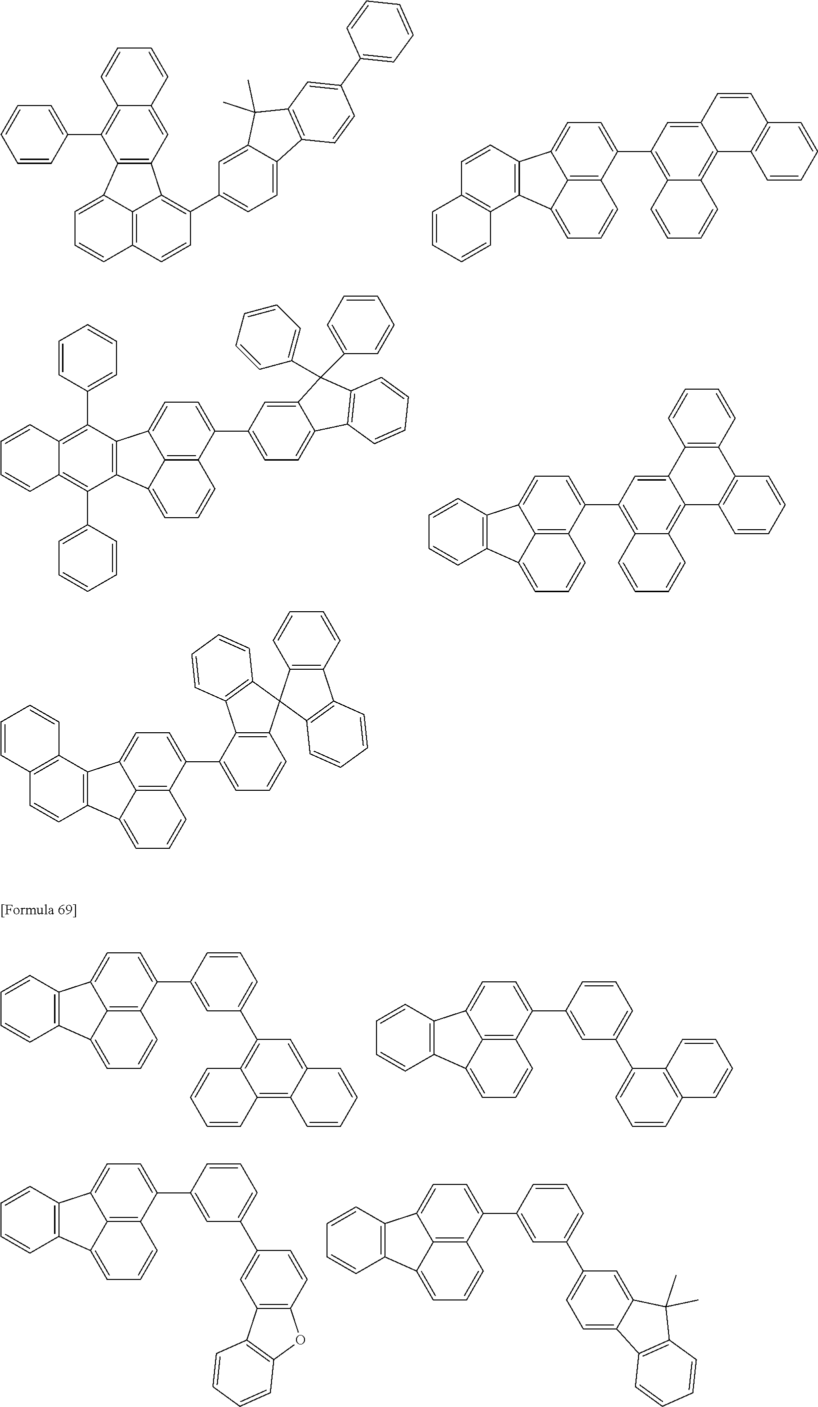

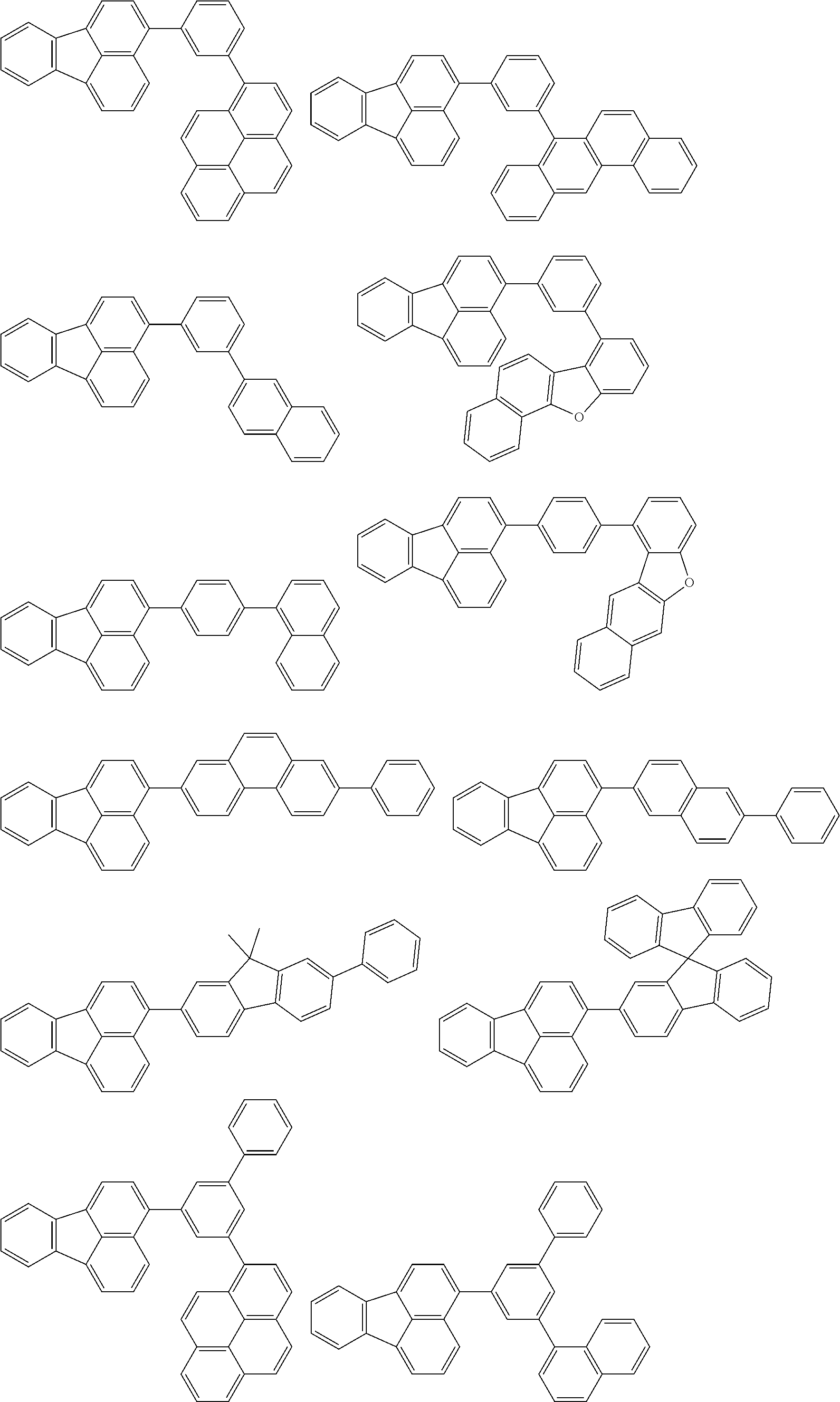

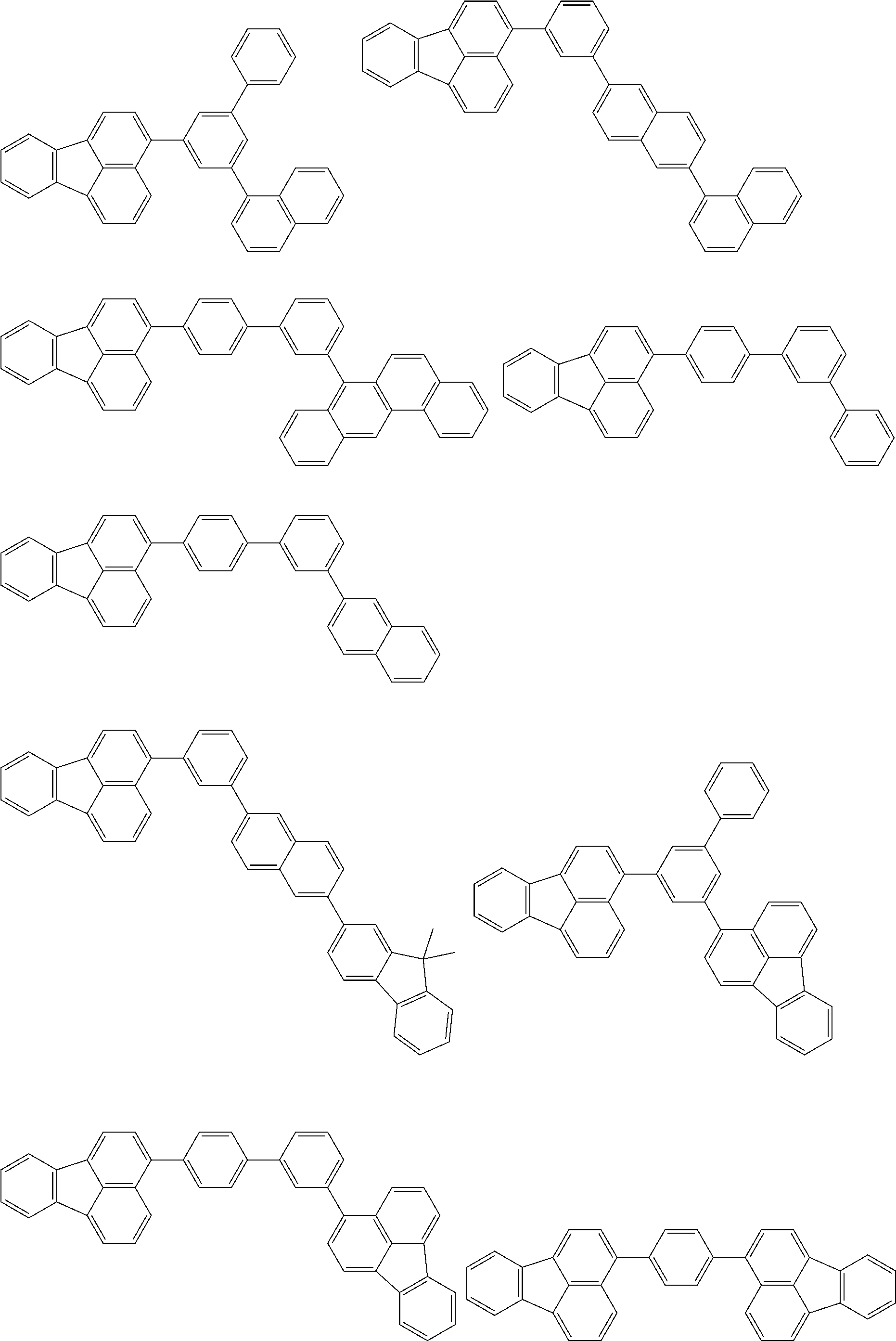

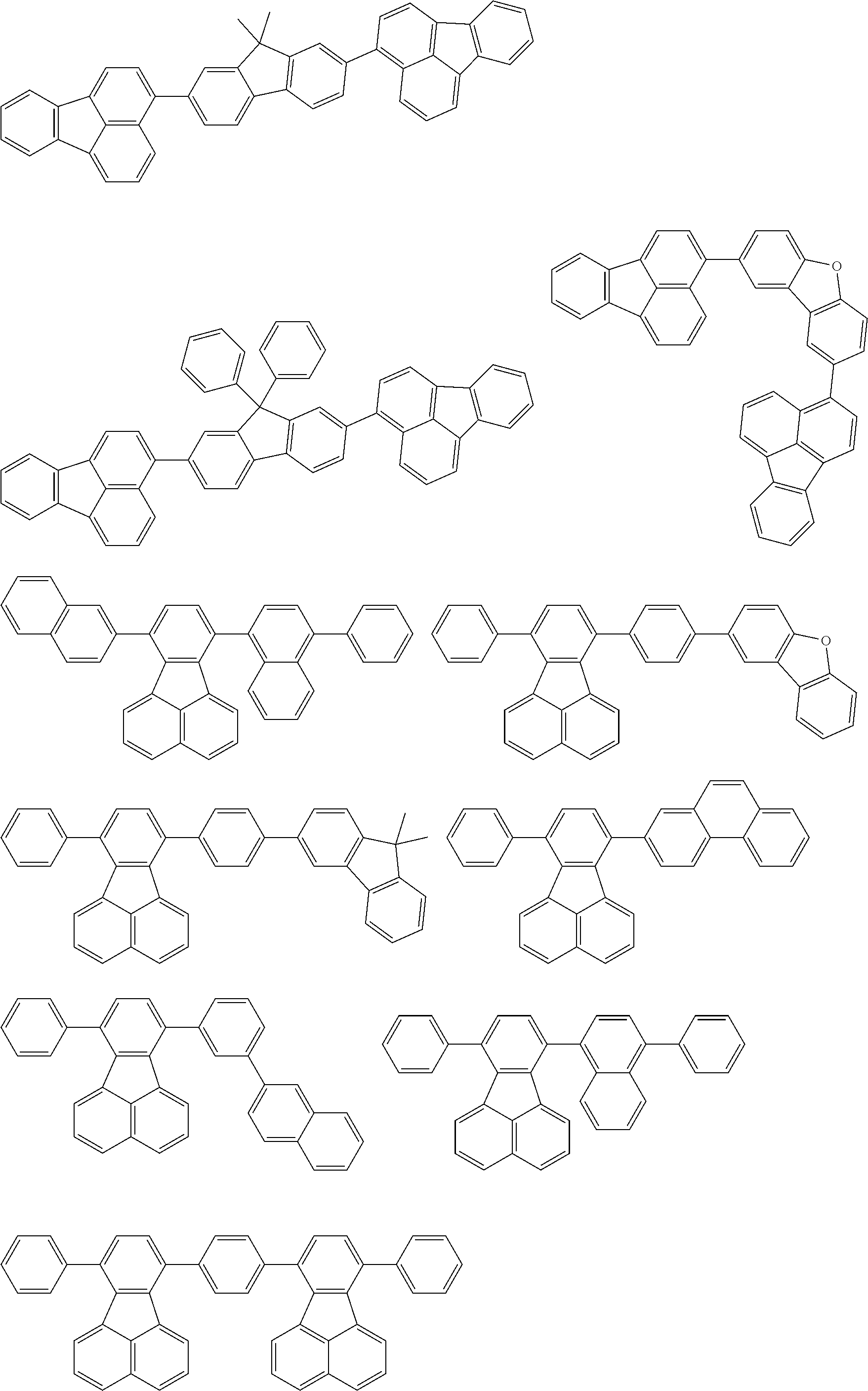

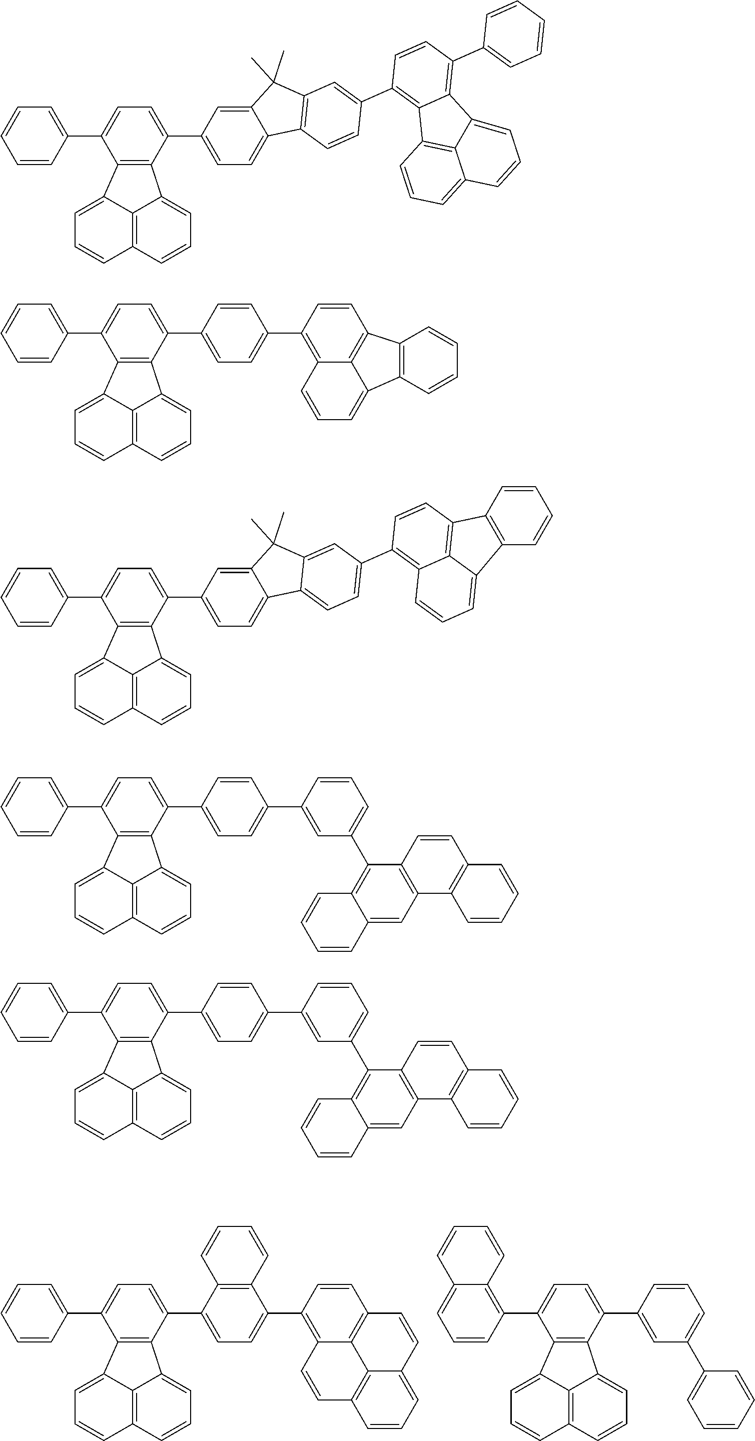

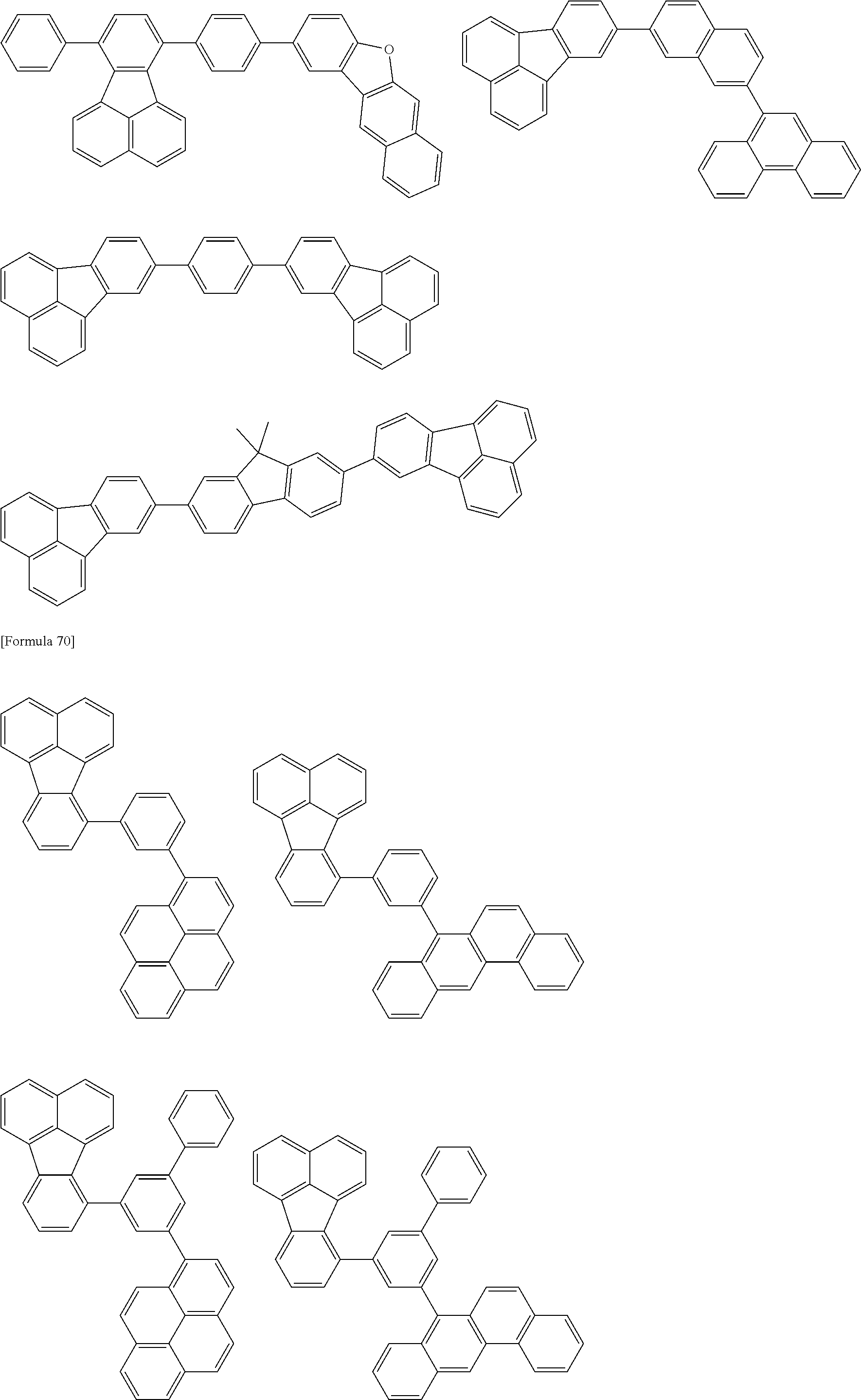

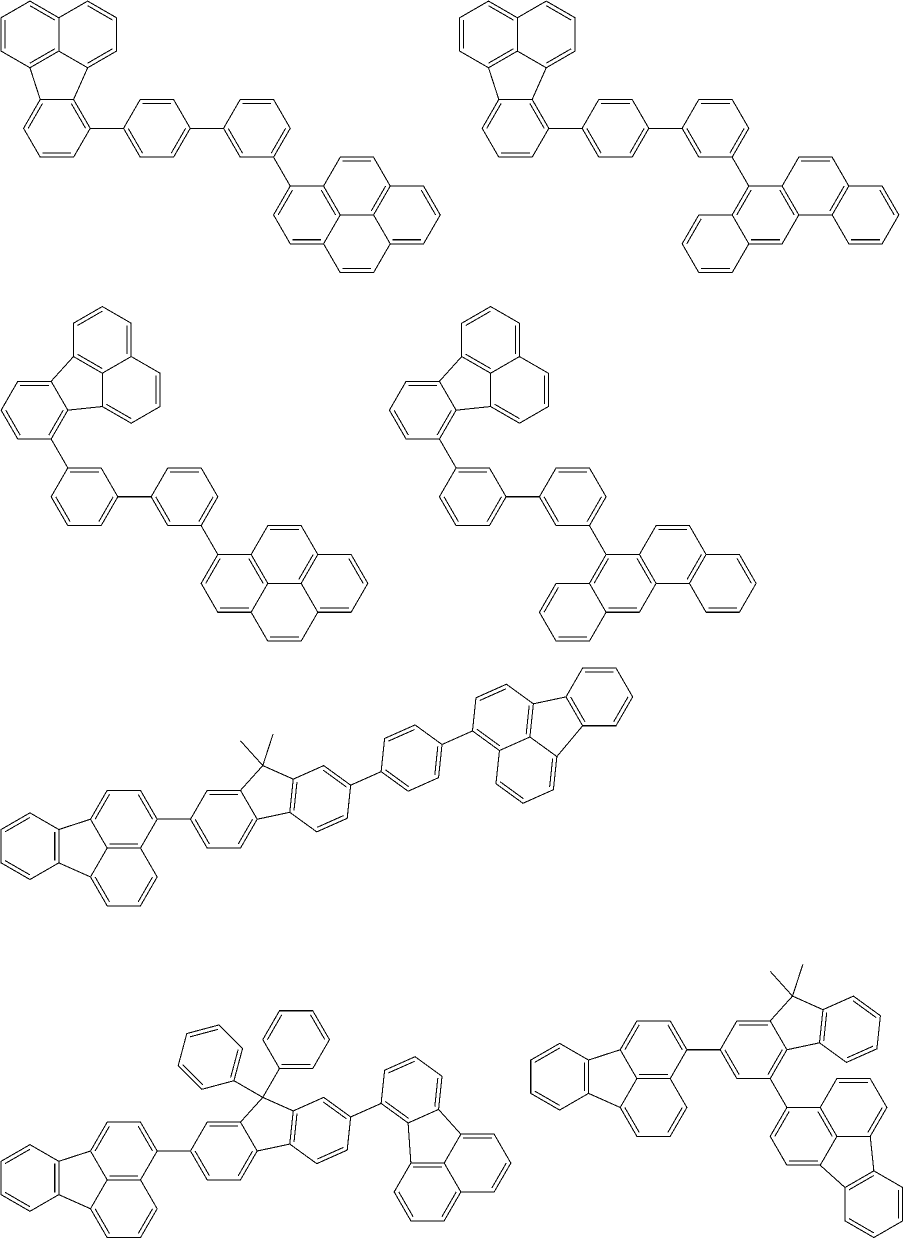

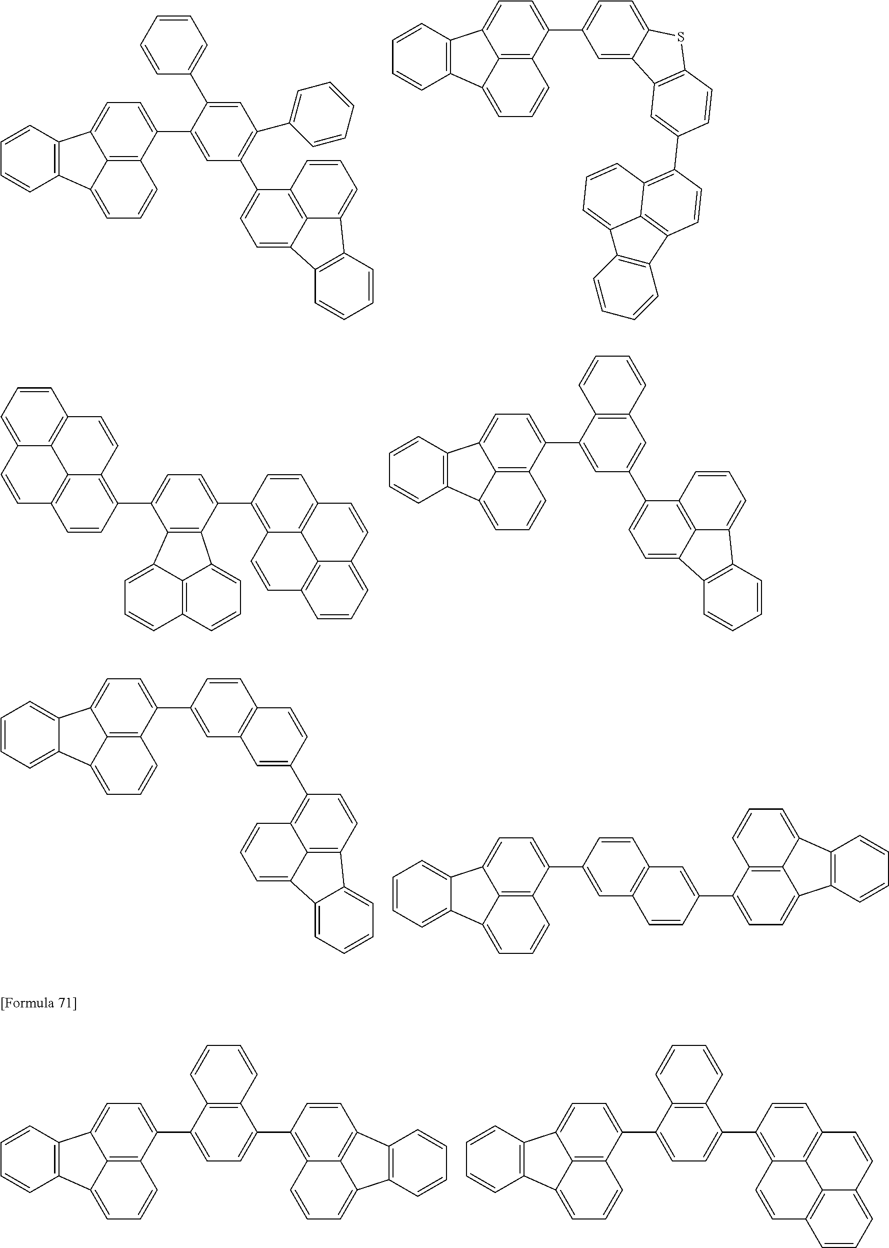

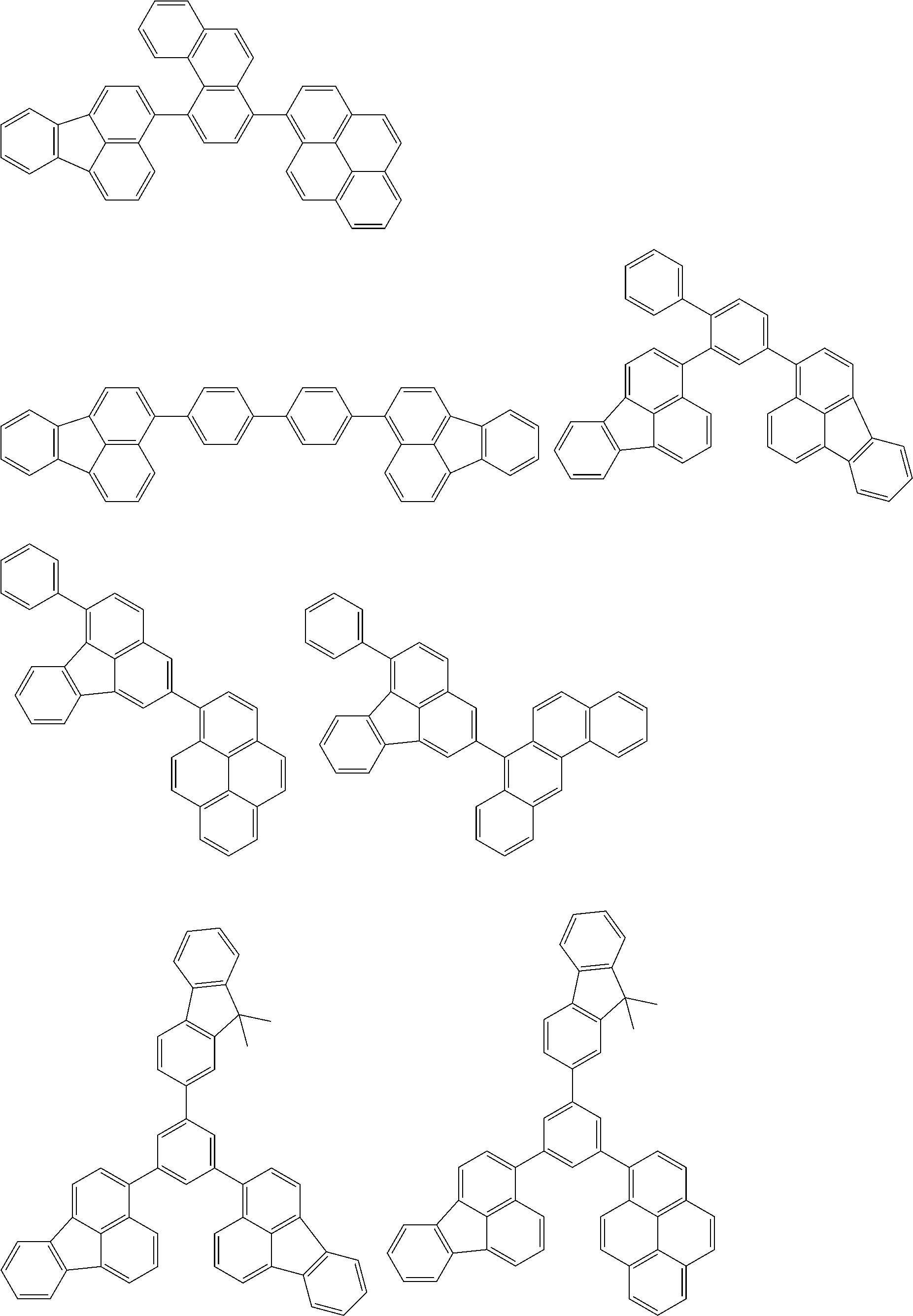

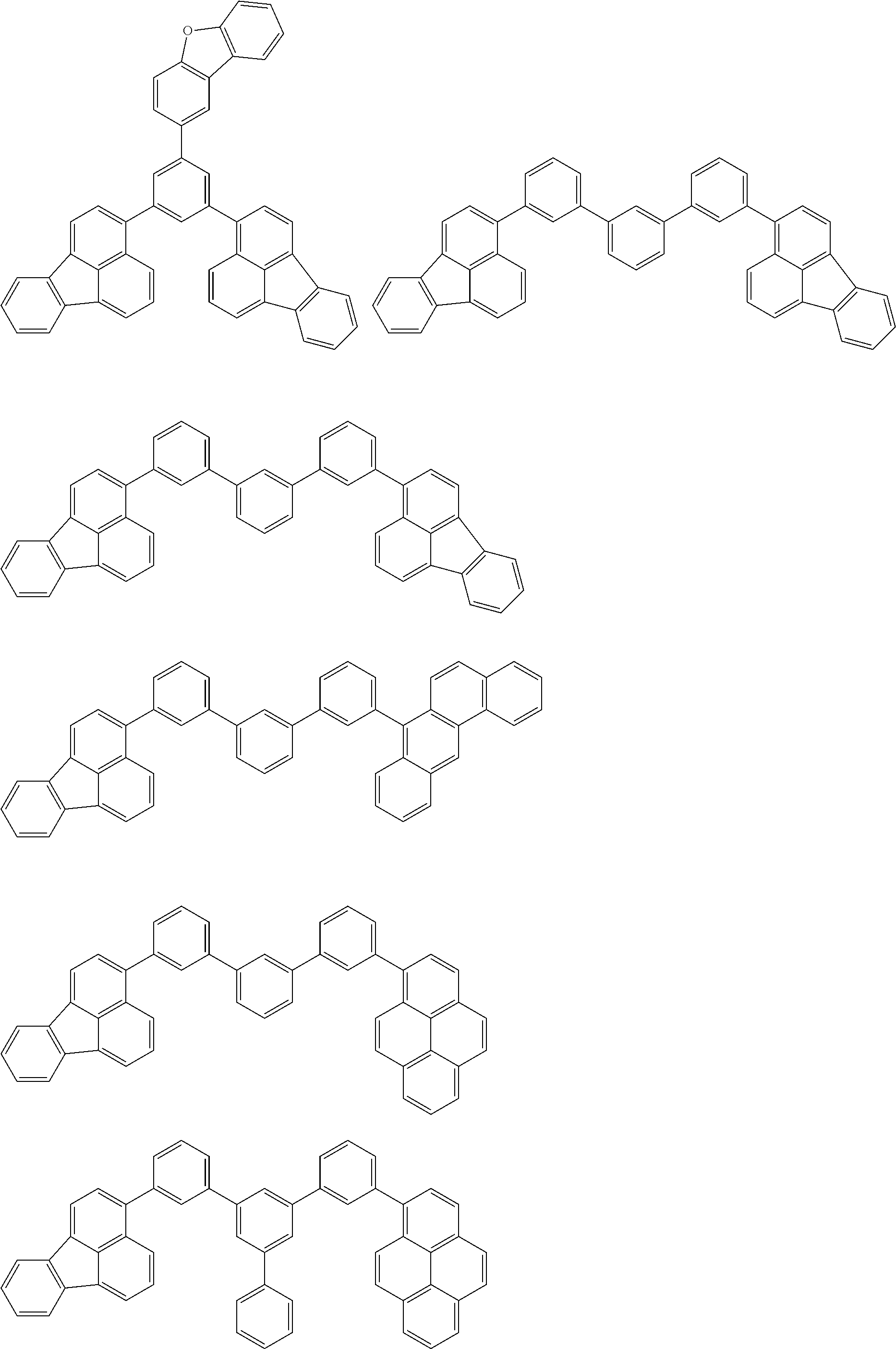

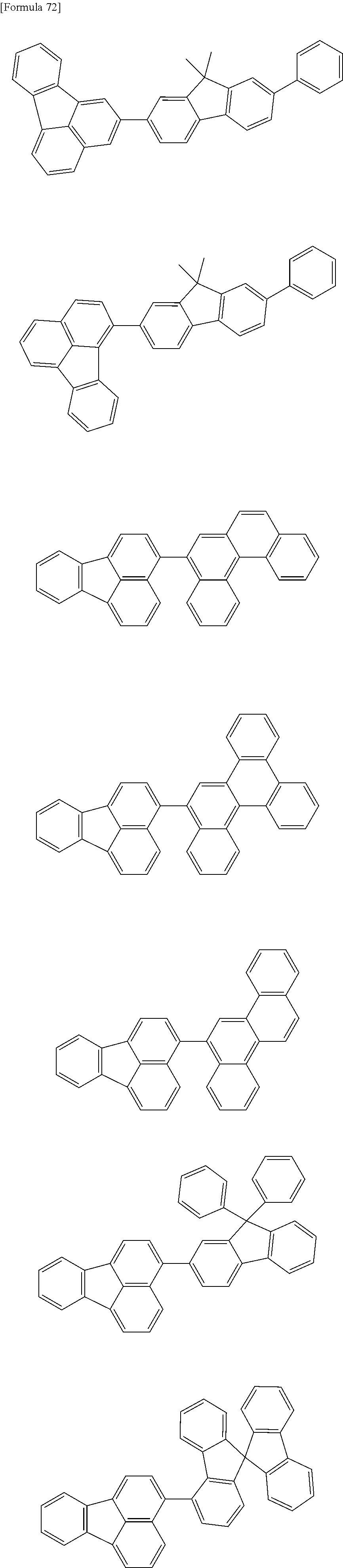

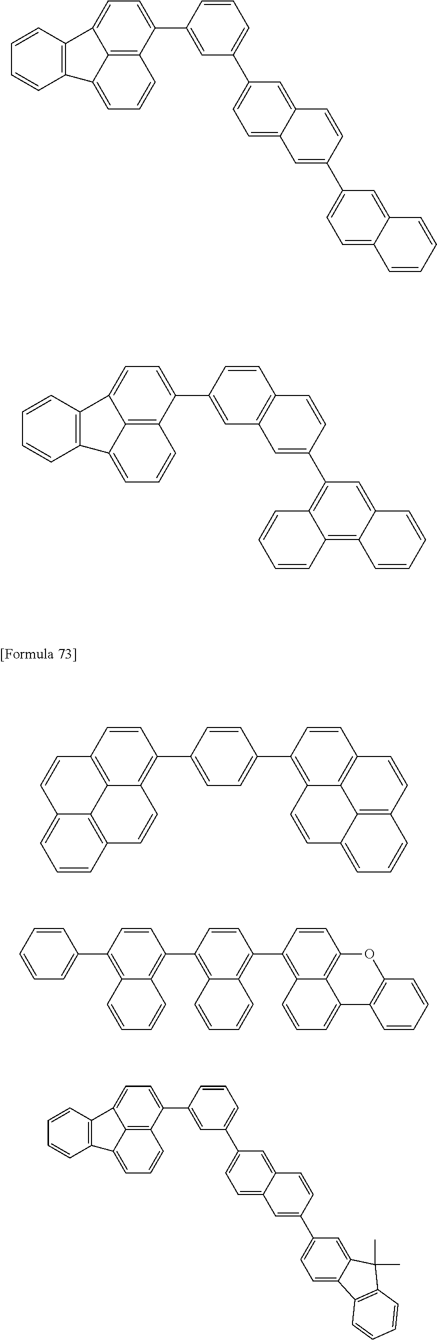

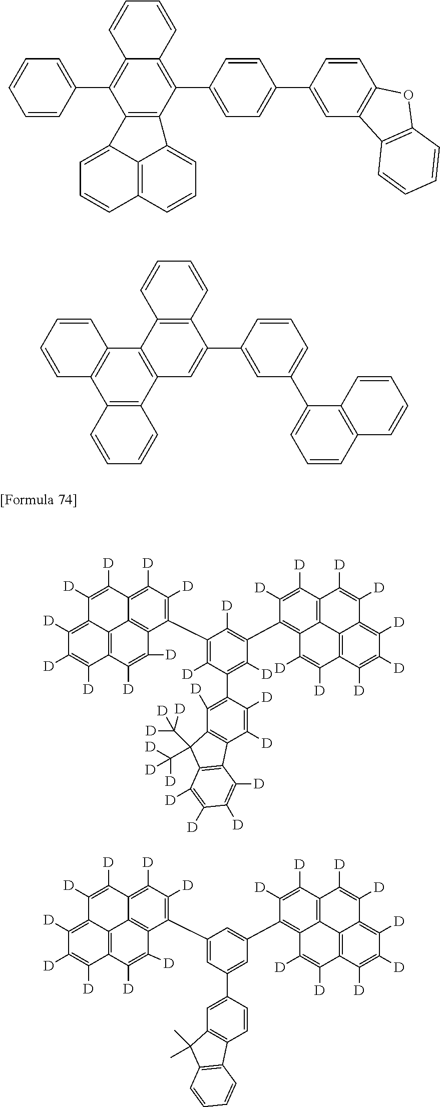

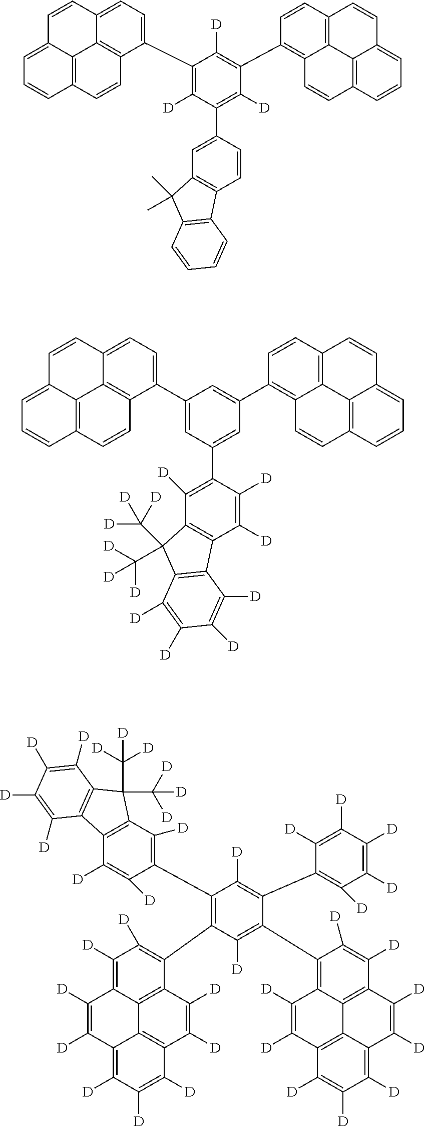

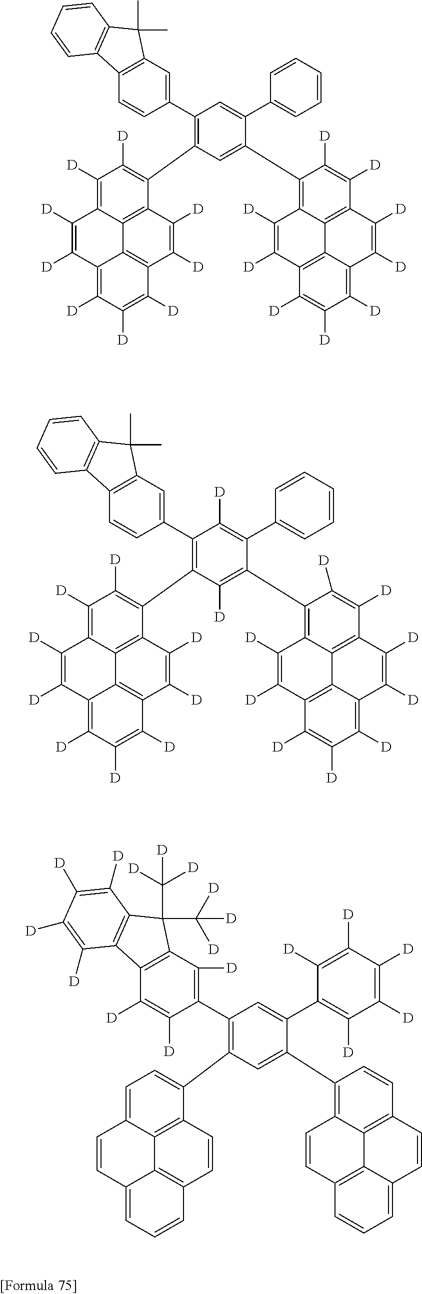

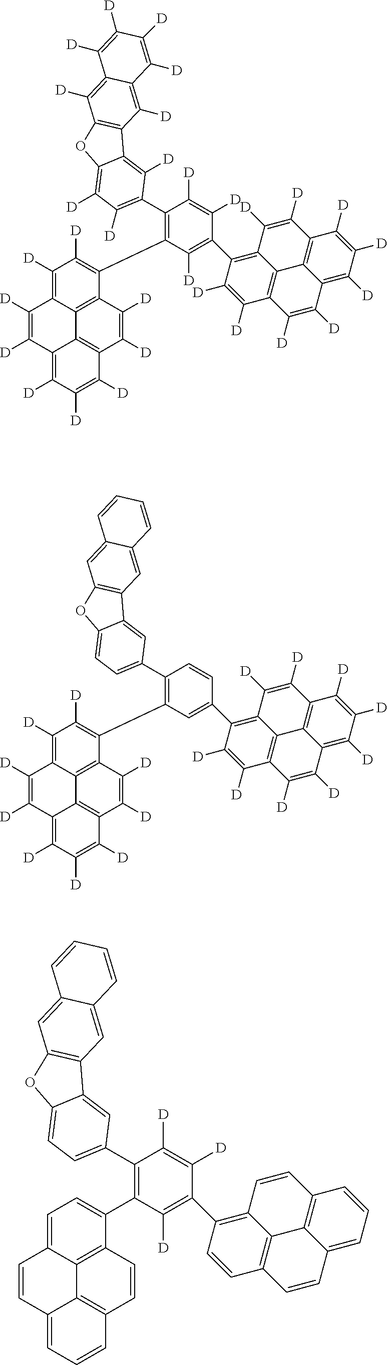

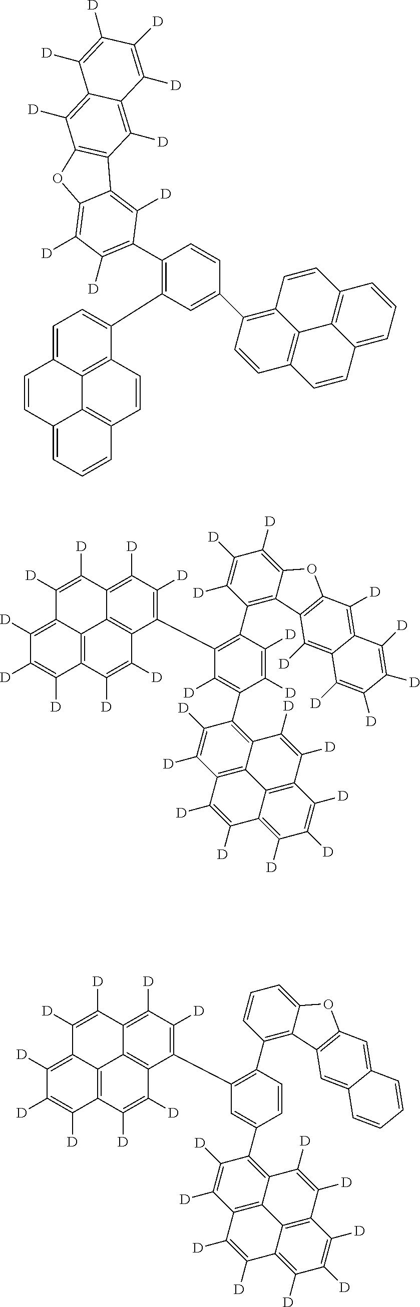

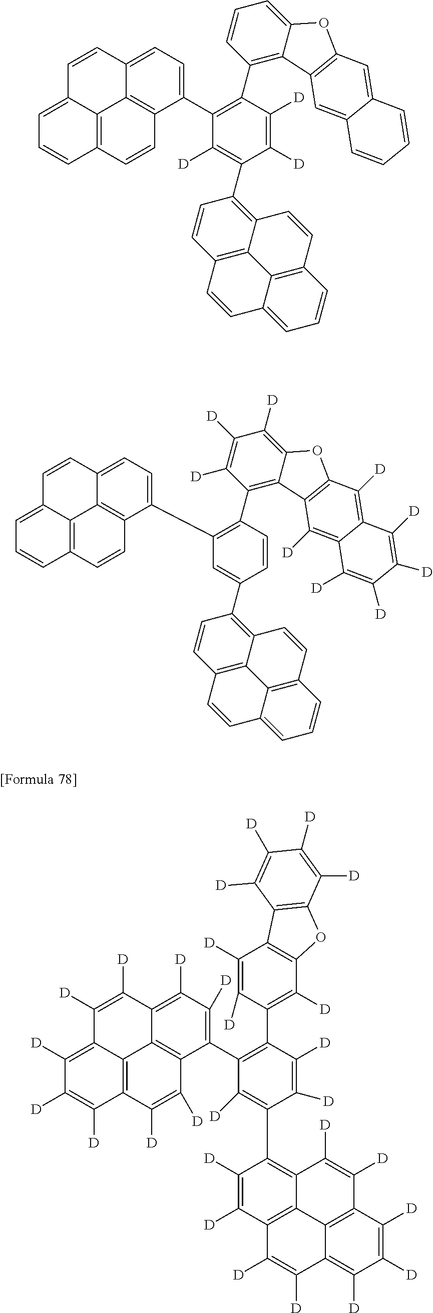 [SMILES: [2H]c1c([2H])c(-c2cccc3oc4cc5ccccc5cc4c23)c(-c2ccc3ccc4cccc5ccc2c3c45)c([2H])c1-c1ccc2ccc3cccc4ccc1c2c34 ; [2H]c1c([2H])c(-c2ccc(-c3ccc4ccc5cccc6ccc3c4c56)cc2-c2ccc3ccc4cccc5ccc2c3c45)c2c(oc3c([2H])c4c([2H])c([2H])c([2H])c([2H])c4c([2H])c32)c1[2H] ; [2H]c1c([2H])c(-c2c([2H])c([2H])c3c(oc4c([2H])c([2H])c([2H])c([2H])c43)c2[2H])c(-c2c([2H])c([2H])c3c([2H])c([2H])c4c([2H])c([2H])c([2H])c5c([2H])c([2H])c2c3c45)c([2H])c1-c1c([2H])c([2H])c2c([2H])c([2H])c3c([2H])c([2H])c([2H])c4c([2H])c([2H])c1c2c34]

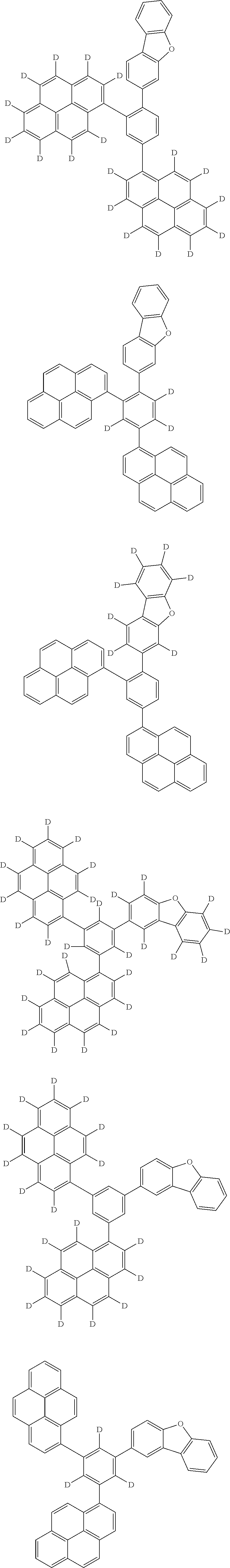

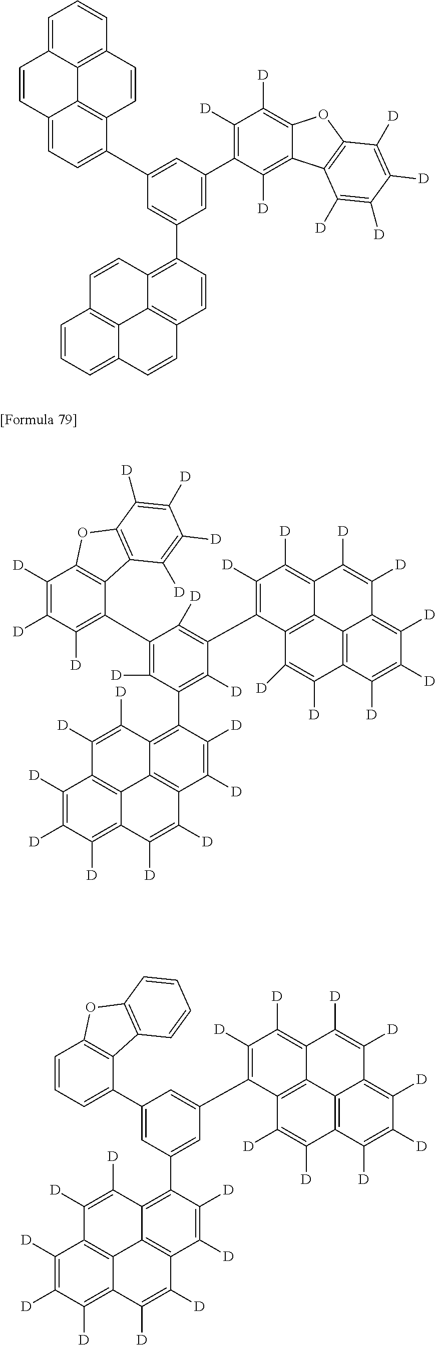

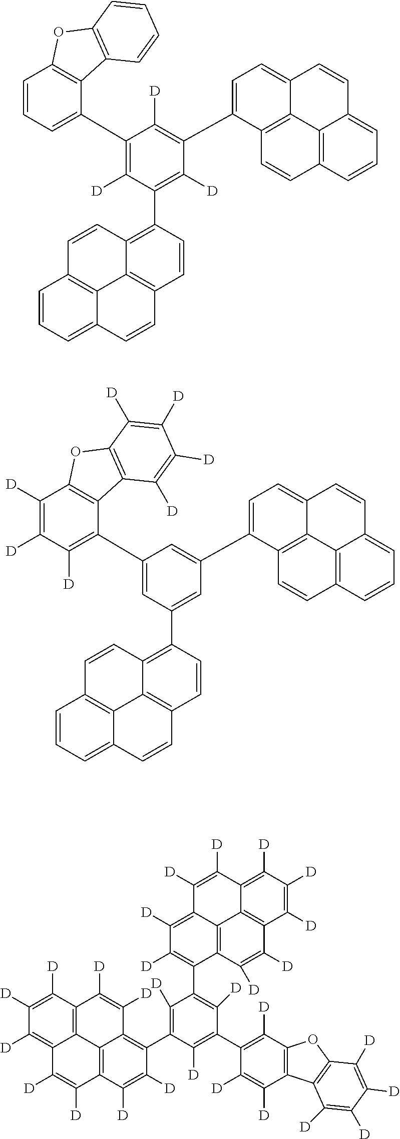 [SMILES: [2H]c1c(-c2ccc3ccc4cccc5ccc2c3c45)c([2H])c(-c2cccc3oc4ccccc4c23)c([2H])c1-c1ccc2ccc3cccc4ccc1c2c34 ; [2H]c1c([2H])c([2H])c2c(oc3c([2H])c([2H])c([2H])c(-c4cc(-c5ccc6ccc7cccc8ccc5c6c78)cc(-c5ccc6ccc7cccc8ccc5c6c78)c4)c32)c1[2H] ; [2H]c1c(-c2c([2H])c([2H])c3c(oc4c([2H])c([2H])c([2H])c([2H])c43)c2[2H])c([2H])c(-c2c([2H])c([2H])c3c([2H])c([2H])c4c([2H])c([2H])c([2H])c5c([2H])c([2H])c2c3c45)c([2H])c1-c1c([2H])c([2H])c2c([2H])c([2H])c3c([2H])c([2H])c([2H])c4c([2H])c([2H])c1c2c34]

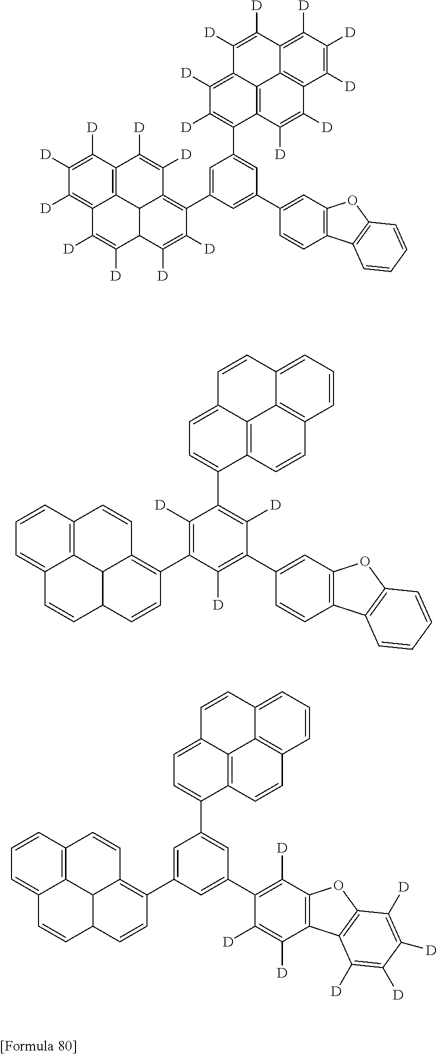 [SMILES: [2H]C1=C([2H])c2c([2H])c([2H])c([2H])c3c2C2C1=C(c1cc(-c4ccc5c(c4)oc4ccccc45)cc(-c4c([2H])c([2H])c5c([2H])c([2H])c6c([2H])c([2H])c([2H])c7c([2H])c([2H])c4c5c67)c1)C([2H])=C([2H])C2C([2H])=C3[2H] ; [2H]c1c(C2=C3C=Cc4cccc5c4C3C(C=C2)C=C5)c([2H])c(-c2ccc3ccc4cccc5ccc2c3c45)c([2H])c1-c1ccc2c(c1)oc1ccccc12 ; [2H]c1c([2H])c([2H])c2c(oc3c([2H])c(-c4cc(C5=C6C=Cc7cccc8c7C6C(C=C5)C=C8)cc(-c5ccc6ccc7cccc8ccc5c6c78)c4)c([2H])c([2H])c32)c1[2H]]

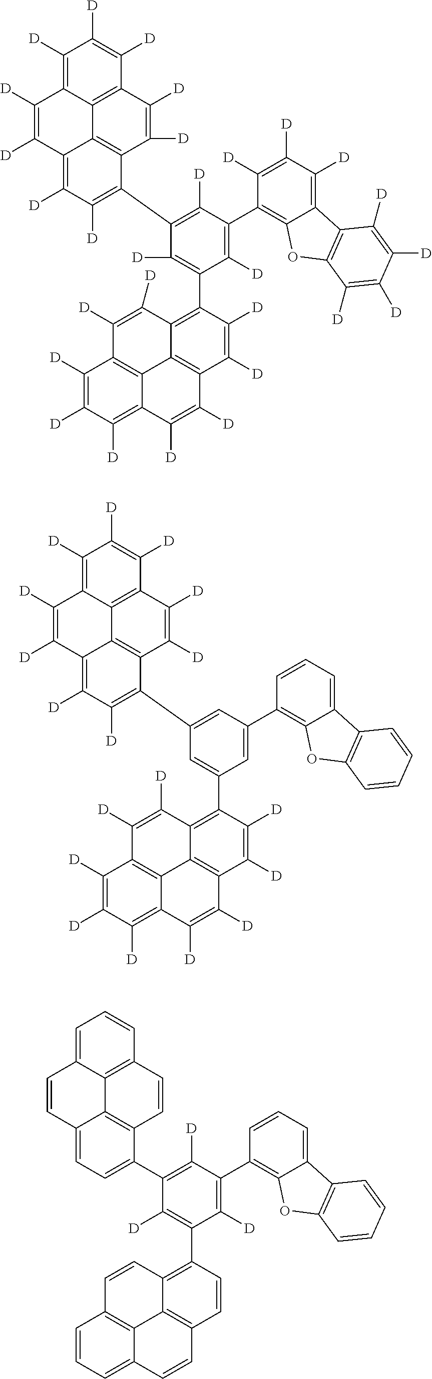

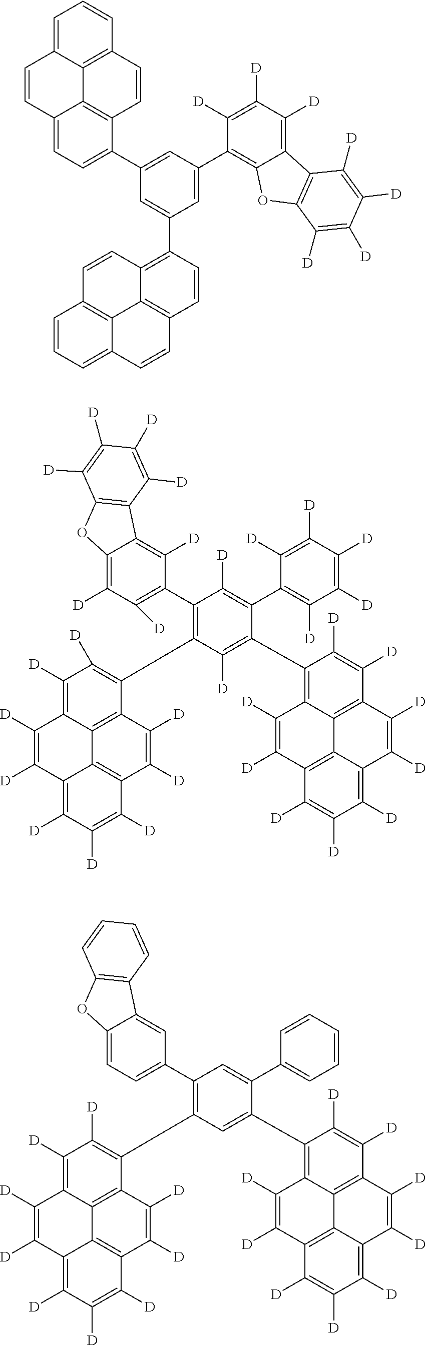

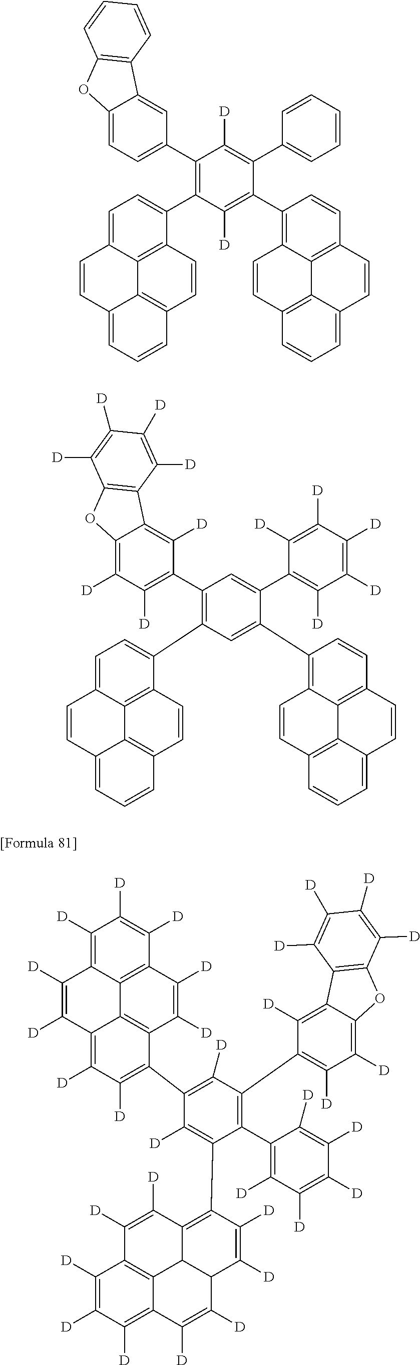

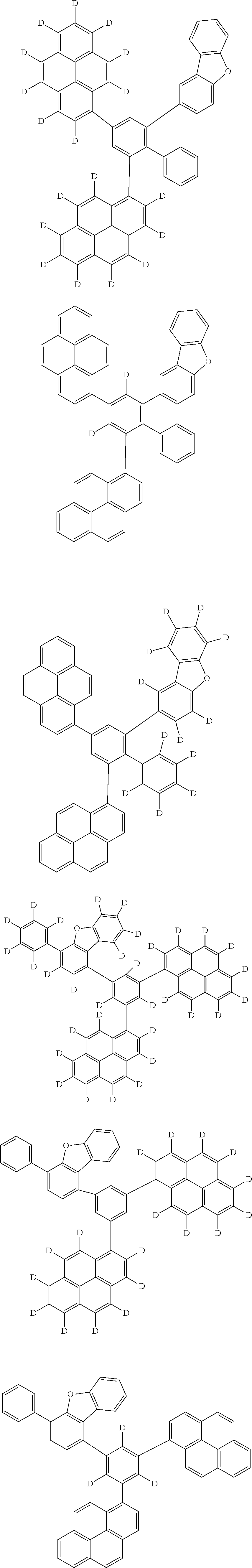

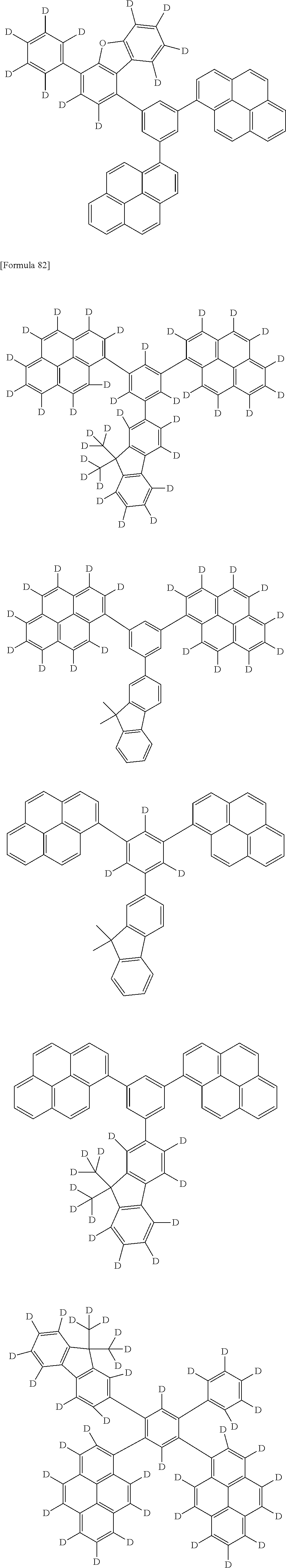

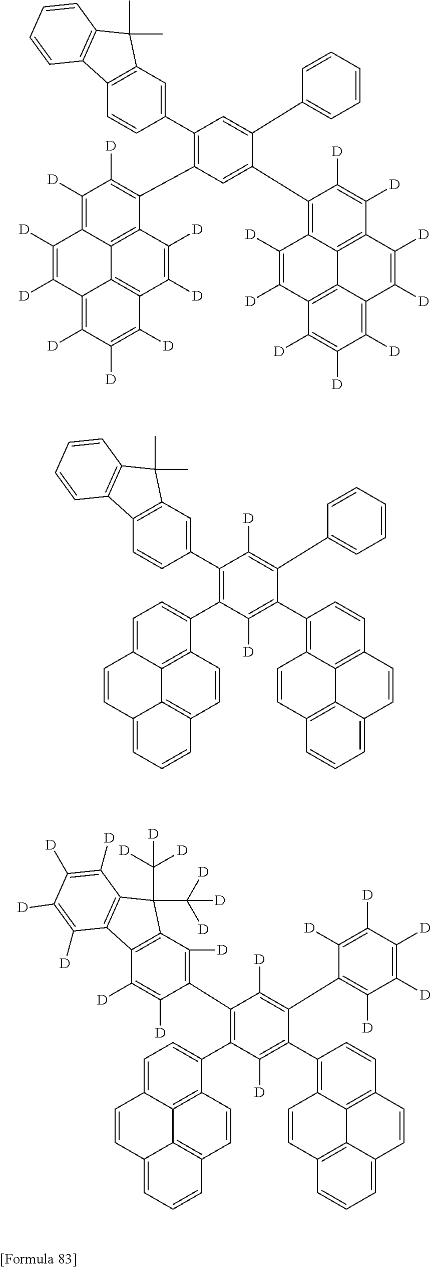

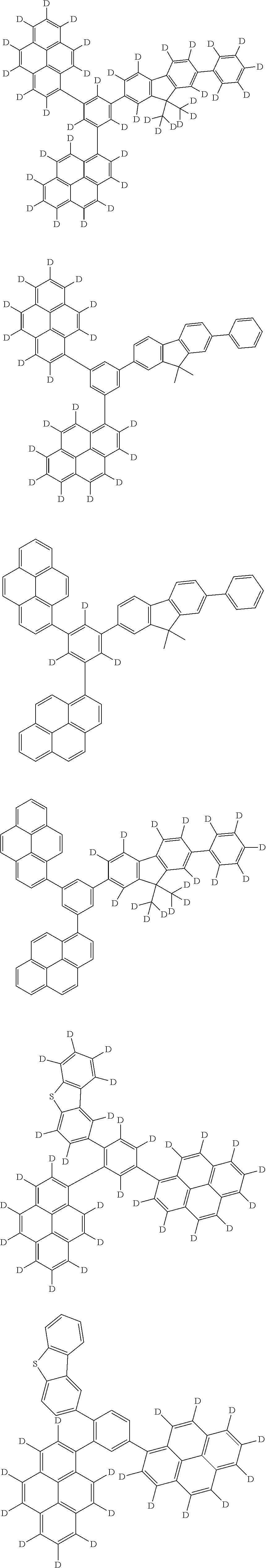

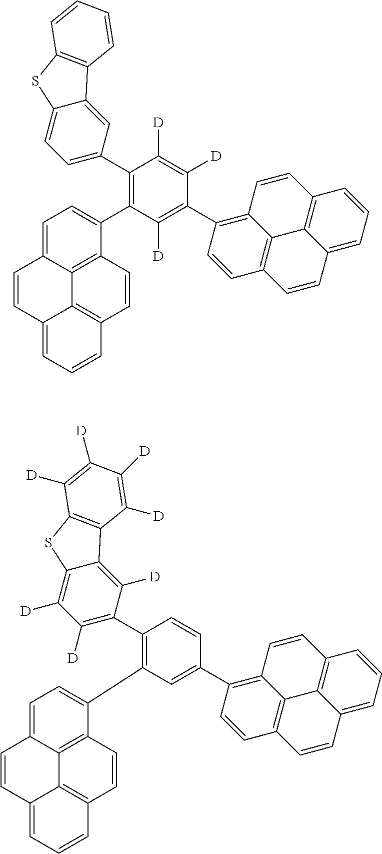 [SMILES: [2H]c1c([2H])c(-c2ccc3sc4ccccc4c3c2)c(-c2ccc3ccc4cccc5ccc2c3c45)c([2H])c1-c1ccc2ccc3cccc4ccc1c2c34 ; [2H]c1c([2H])c([2H])c2c(sc3c([2H])c([2H])c(-c4ccc(-c5ccc6ccc7cccc8ccc5c6c78)cc4-c4ccc5ccc6cccc7ccc4c5c67)c([2H])c32)c1[2H]]

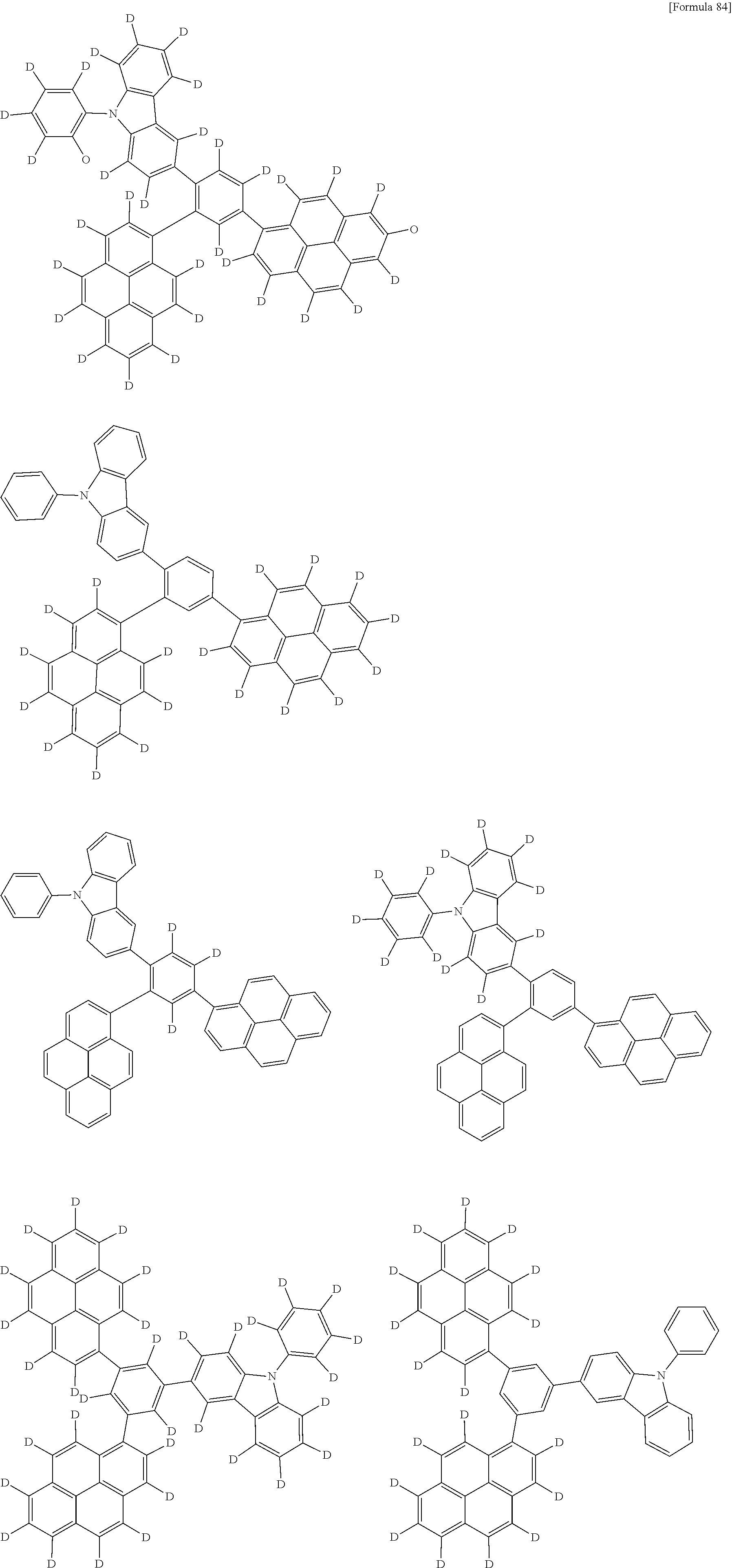

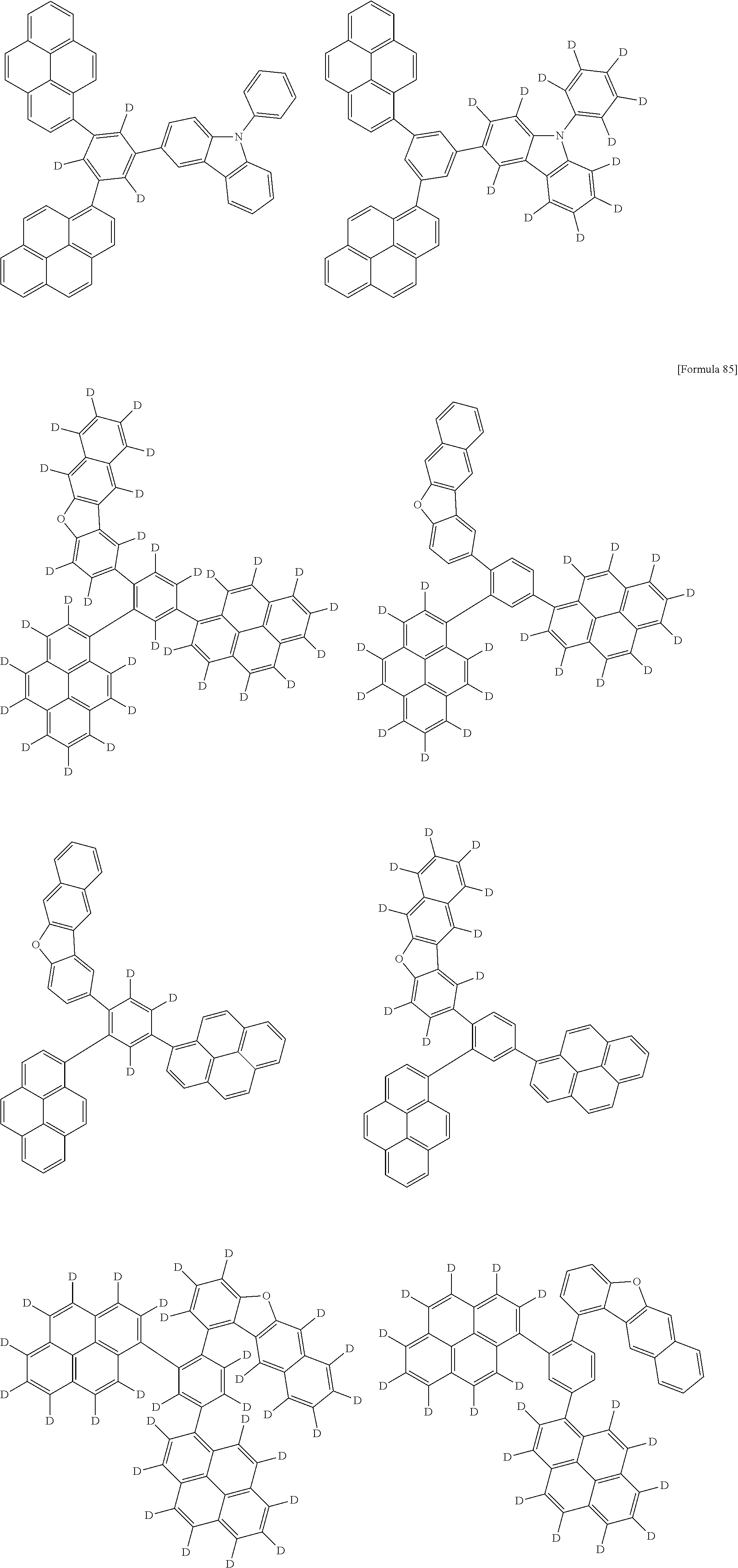

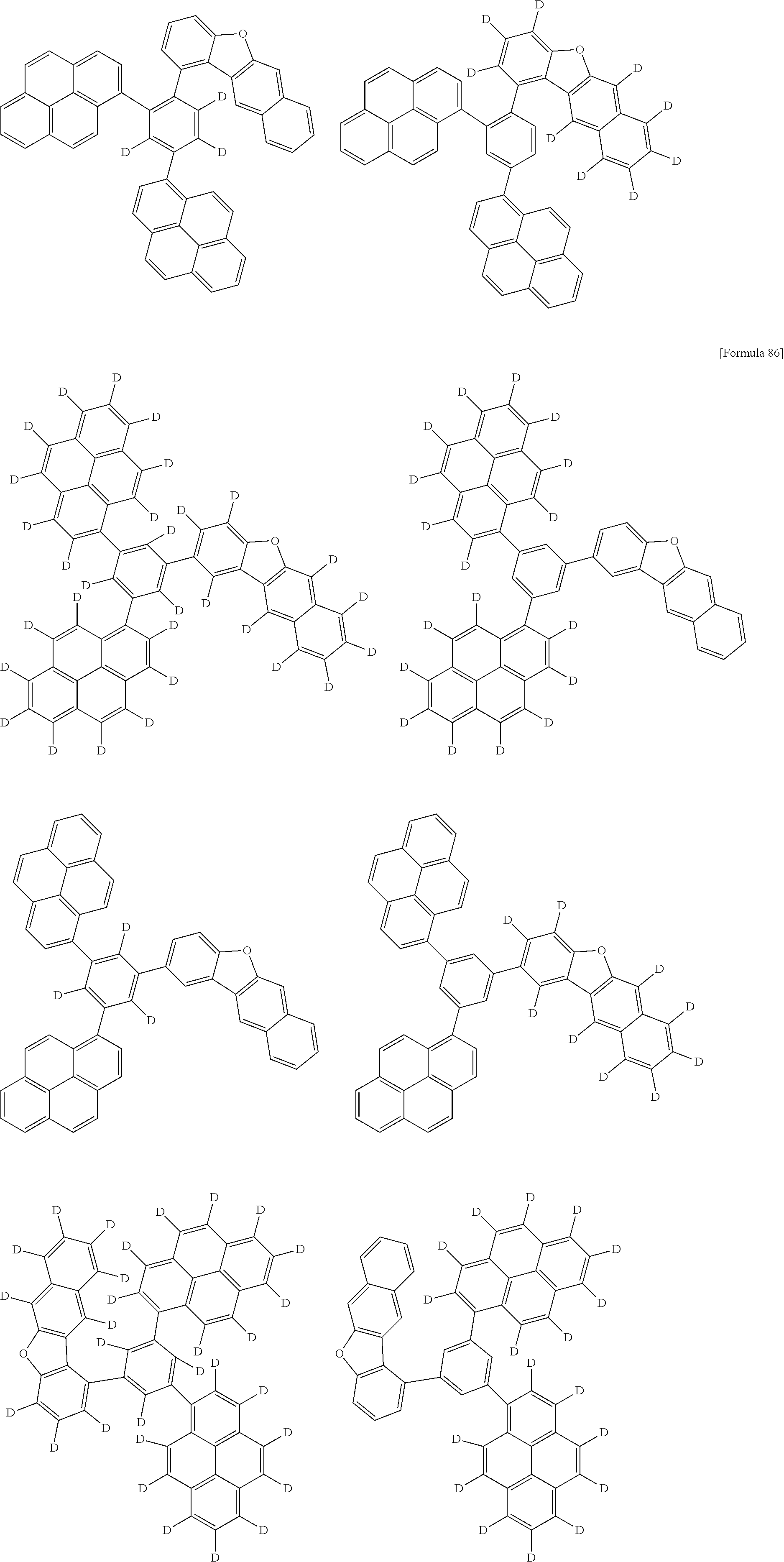

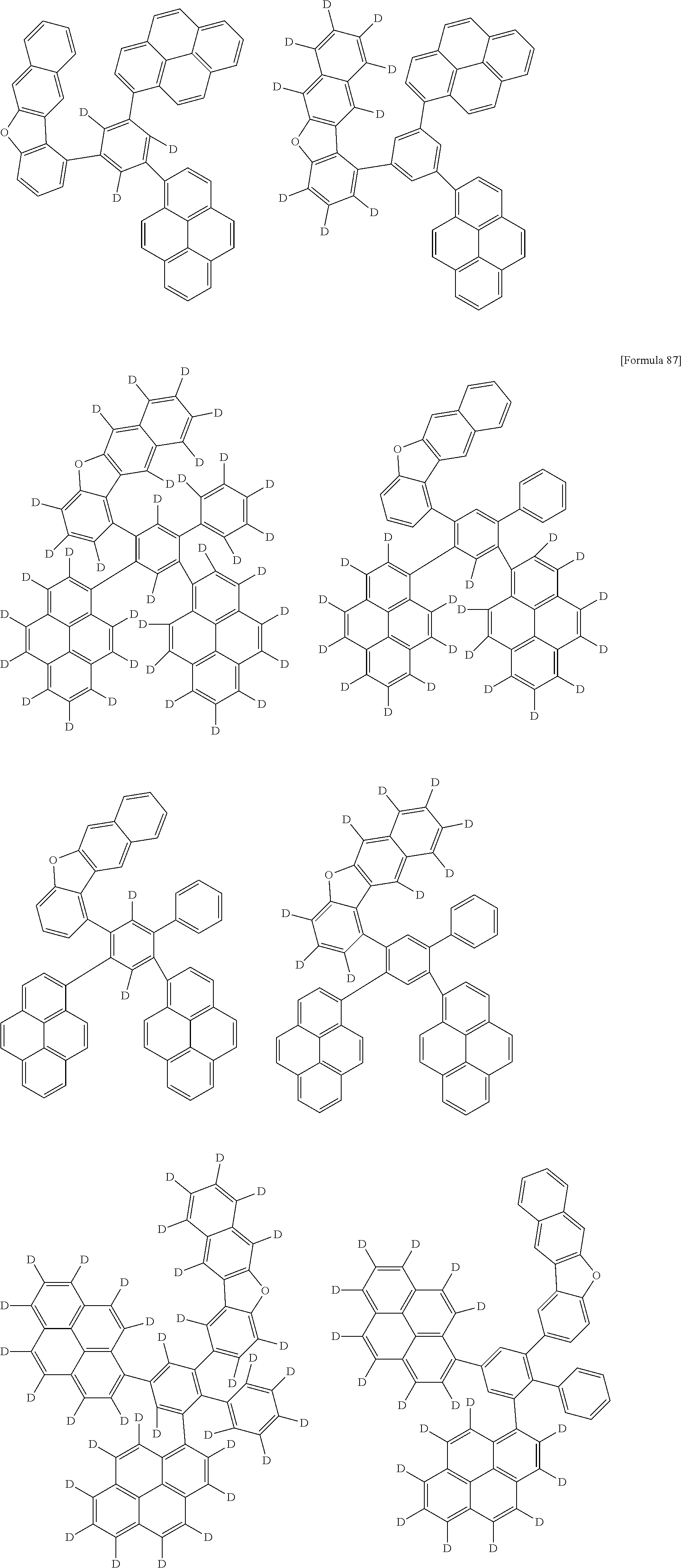

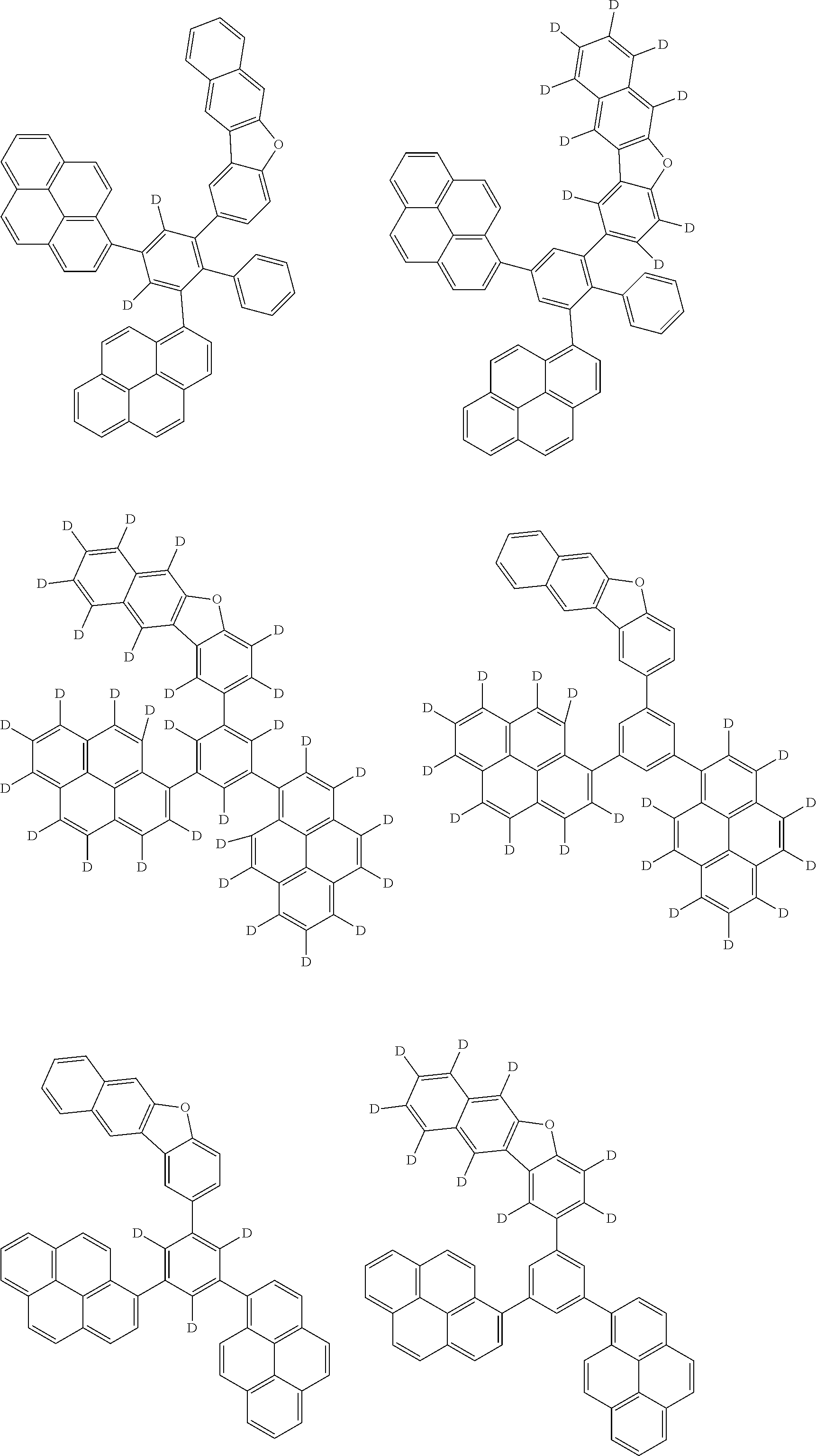

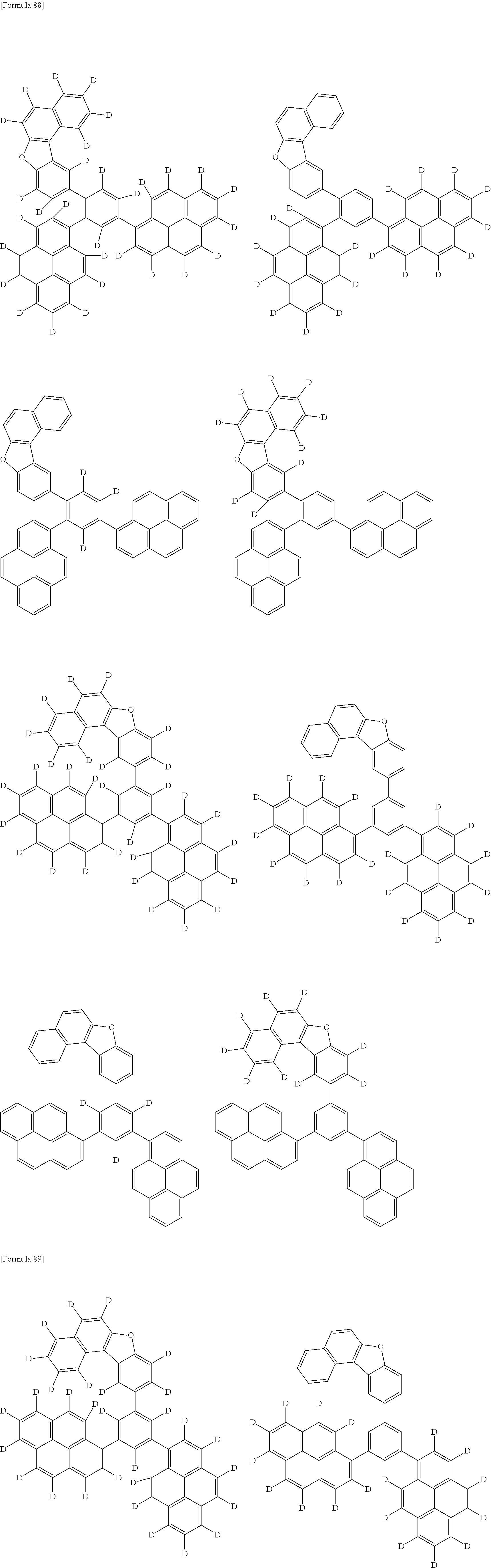

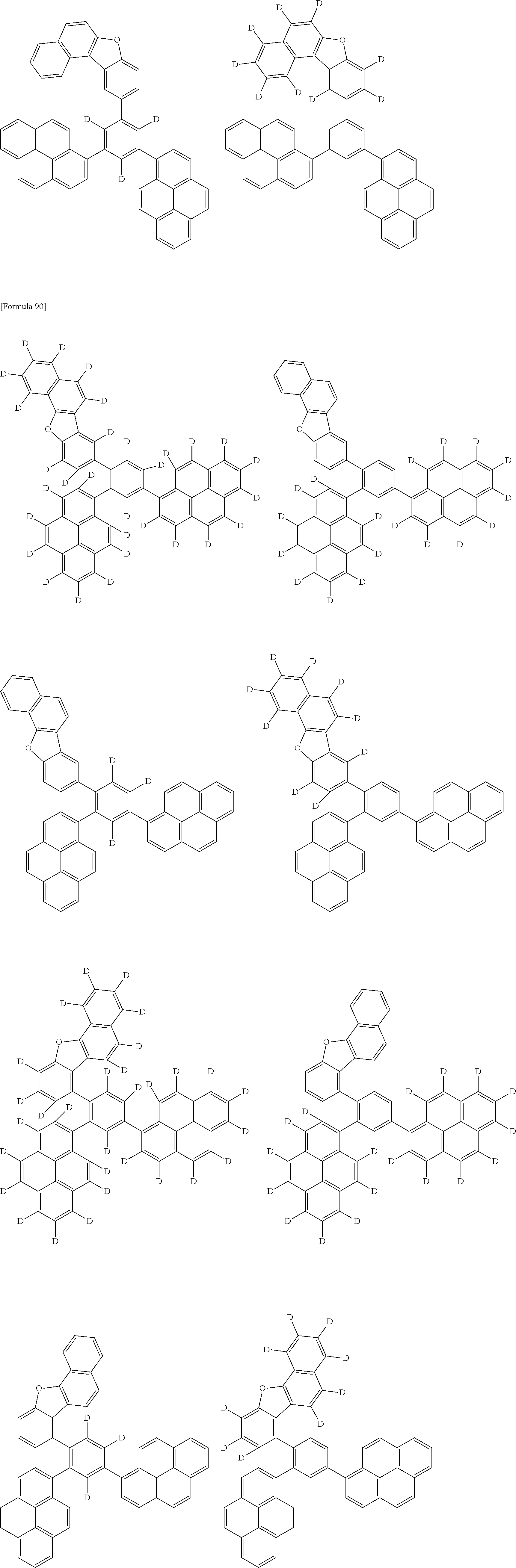

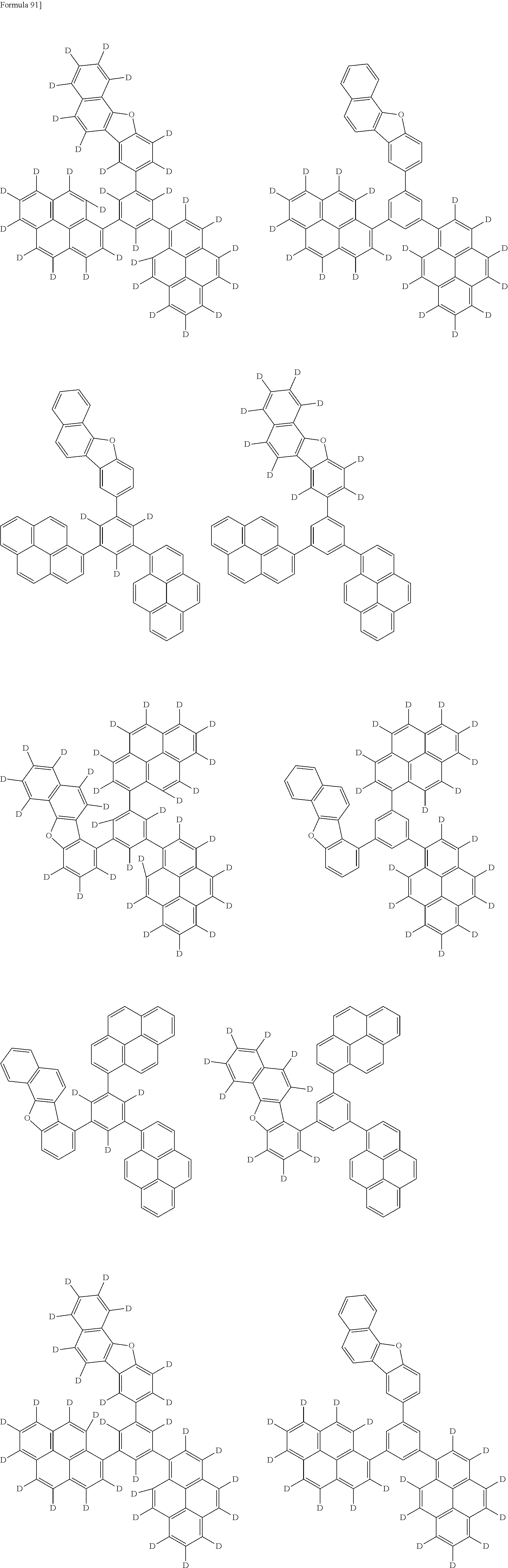

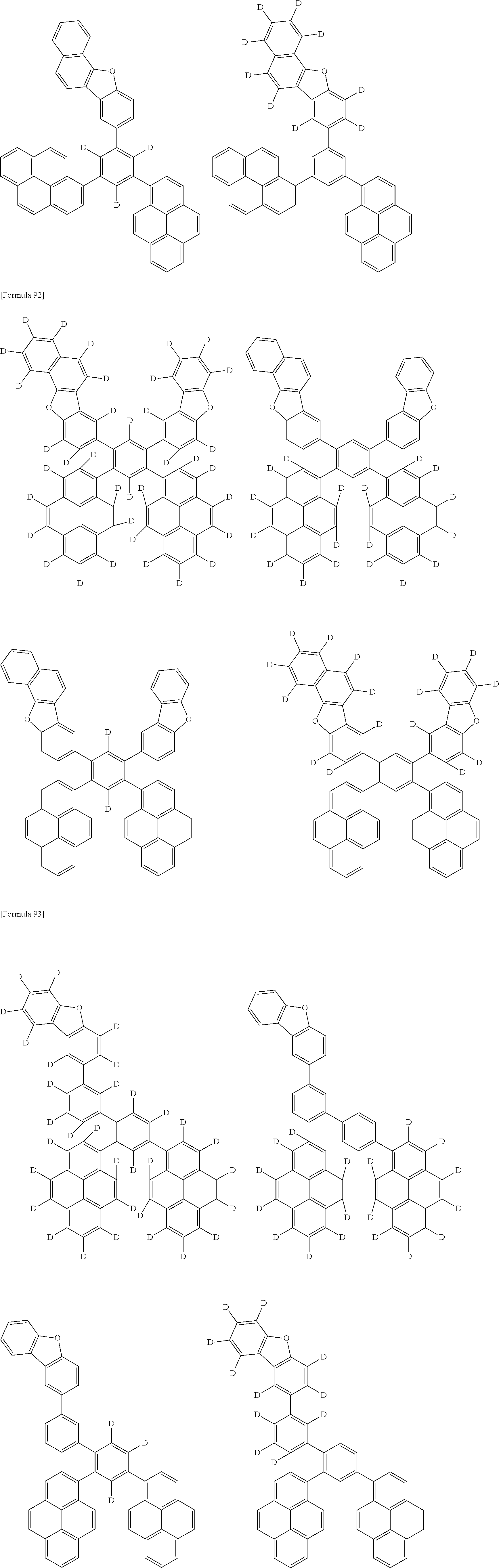

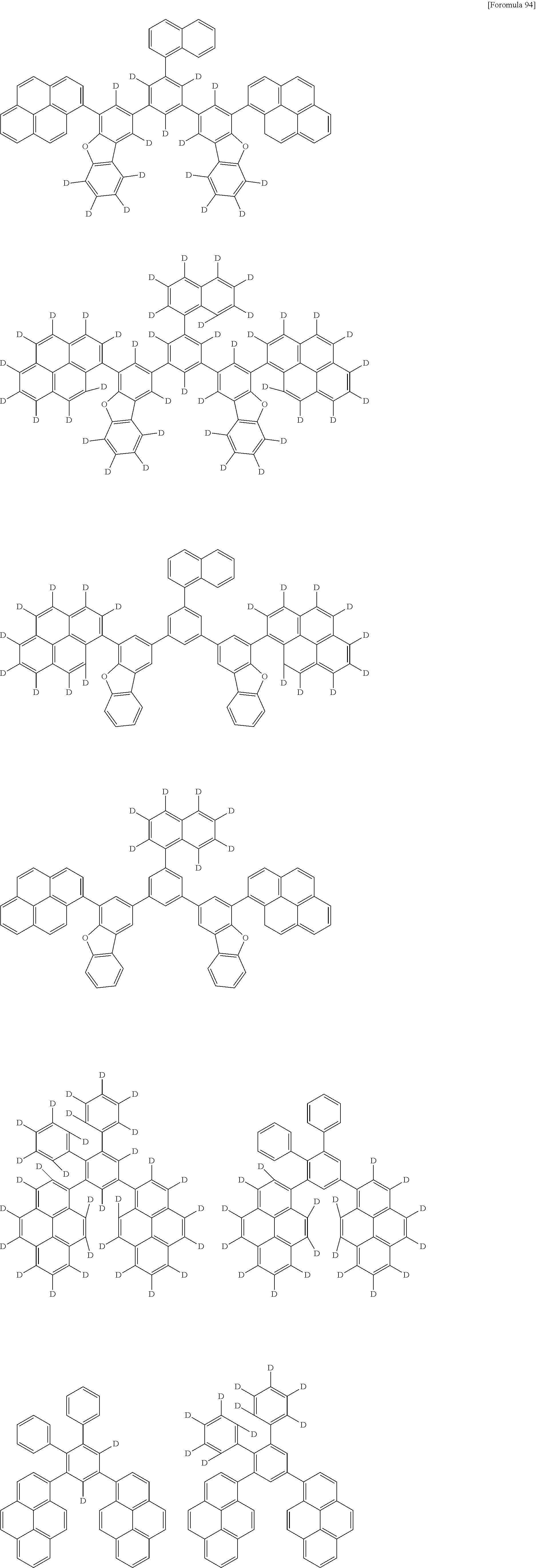

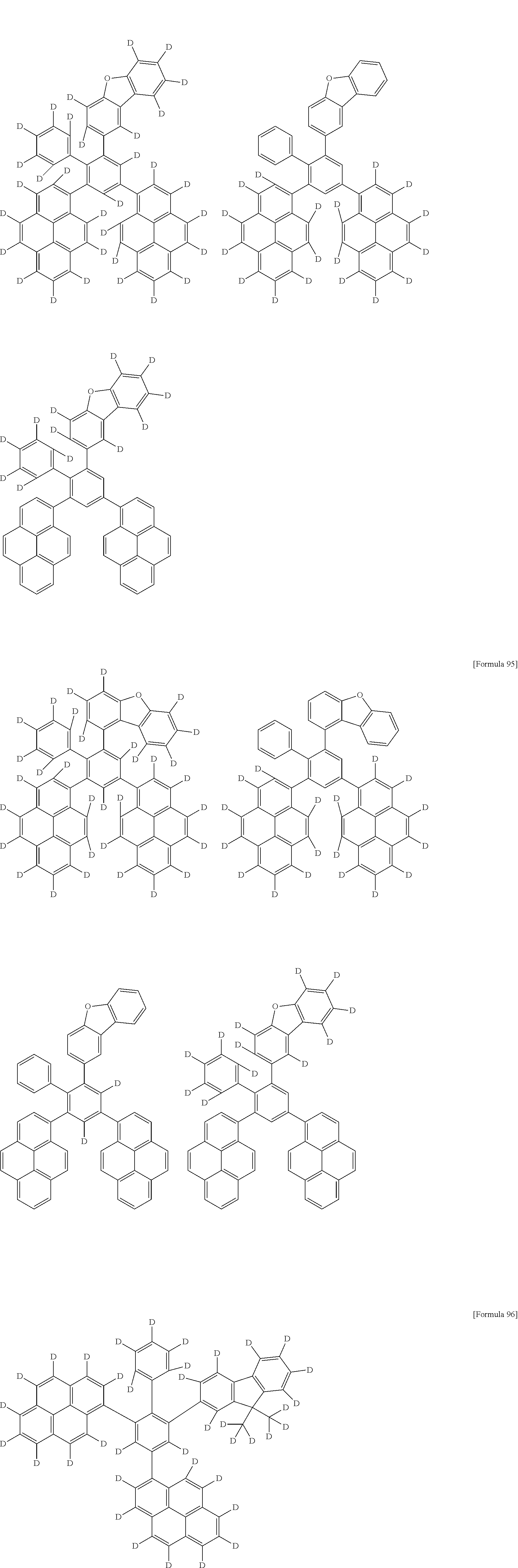

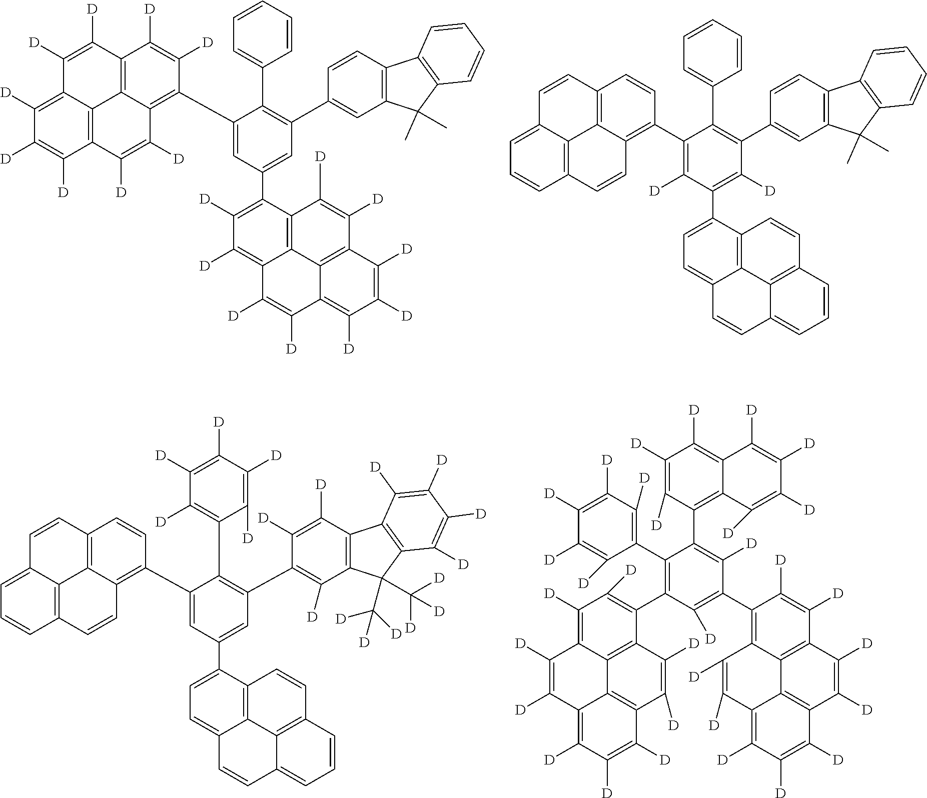

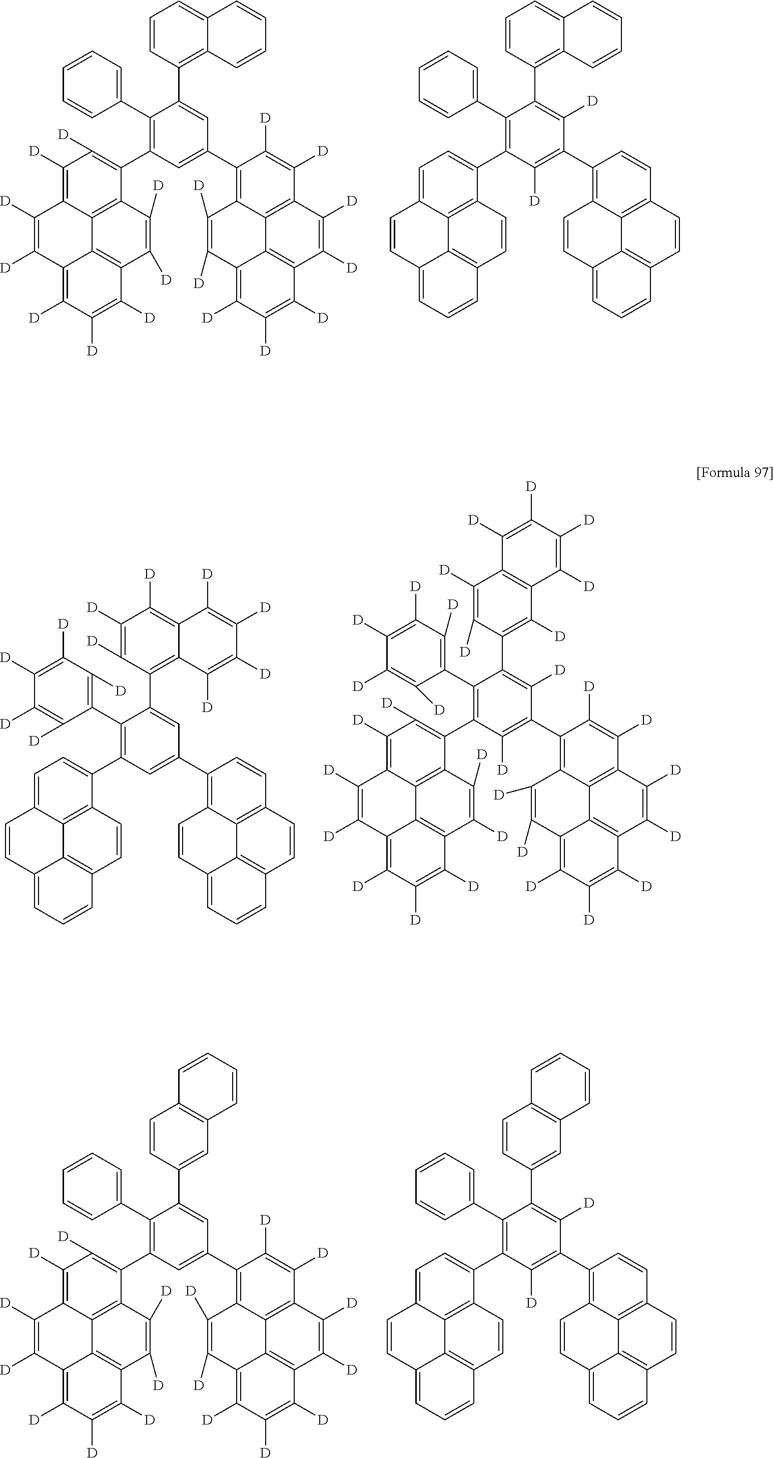

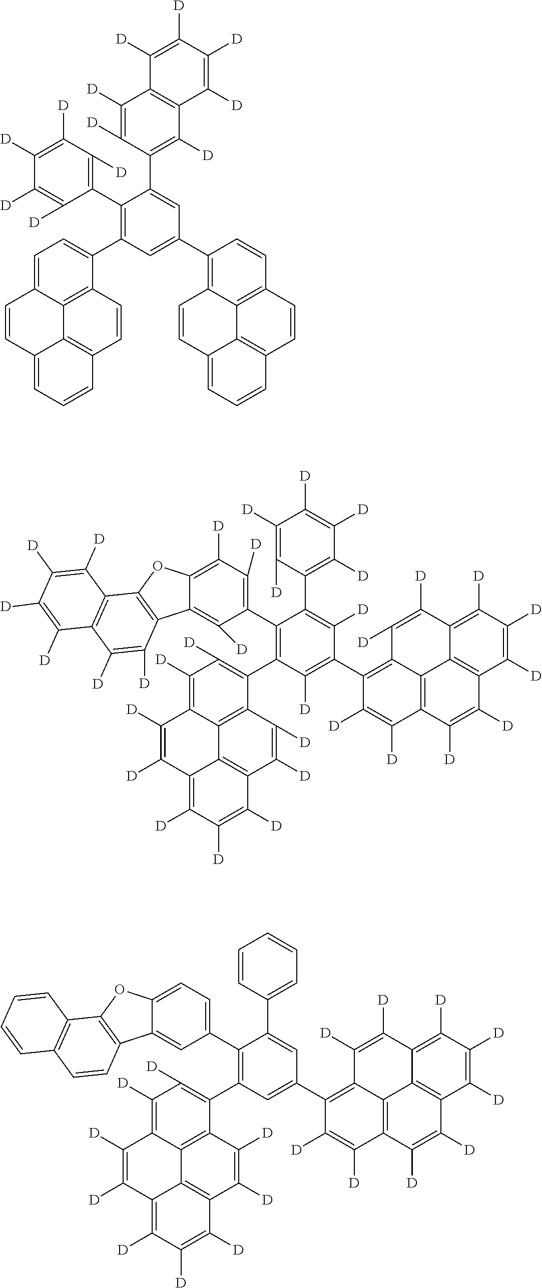

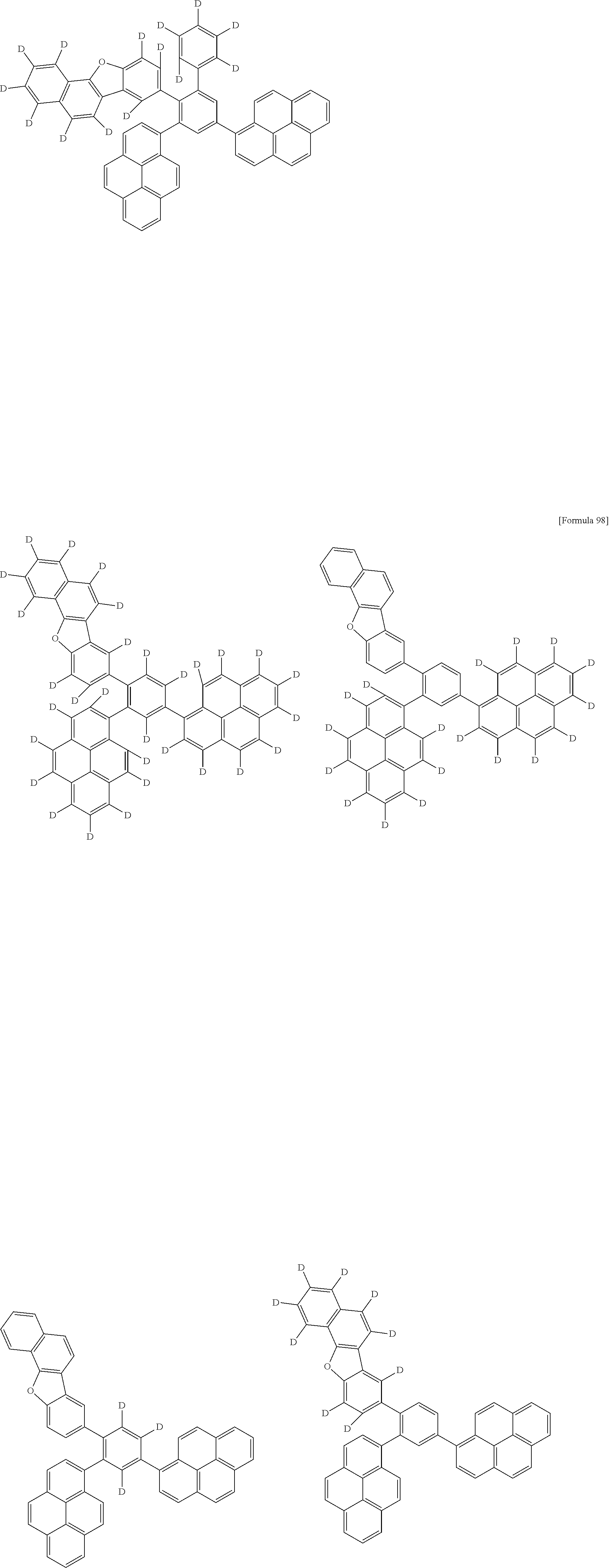

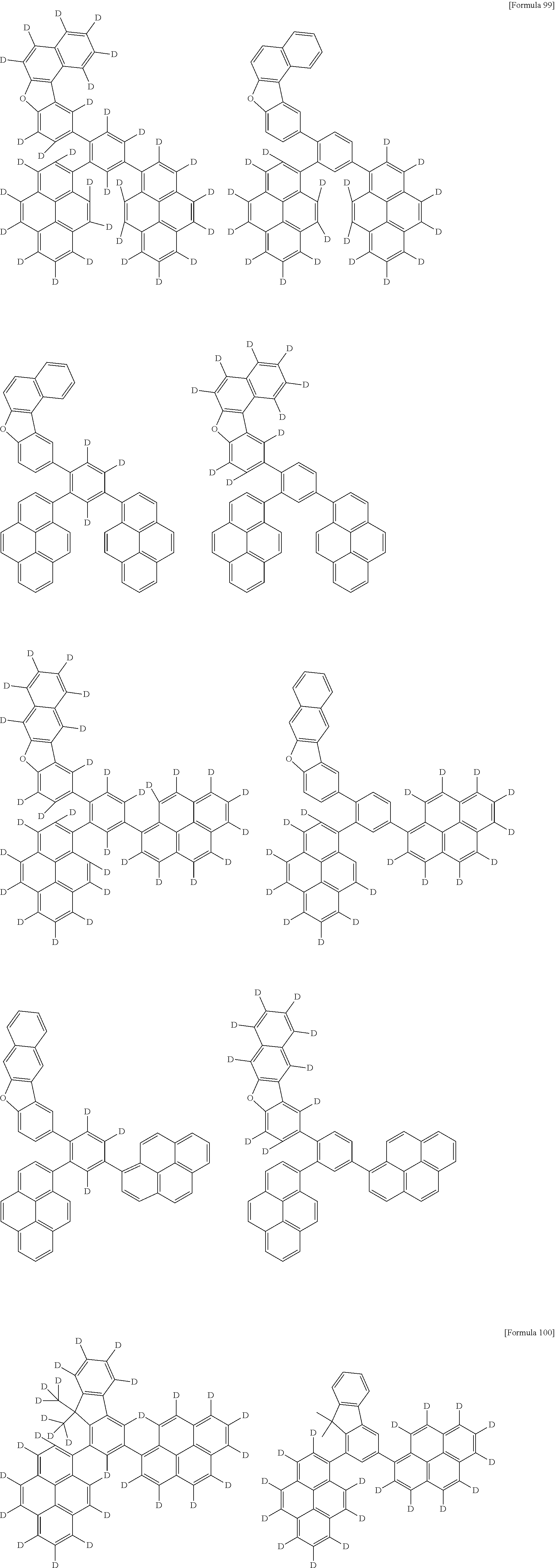

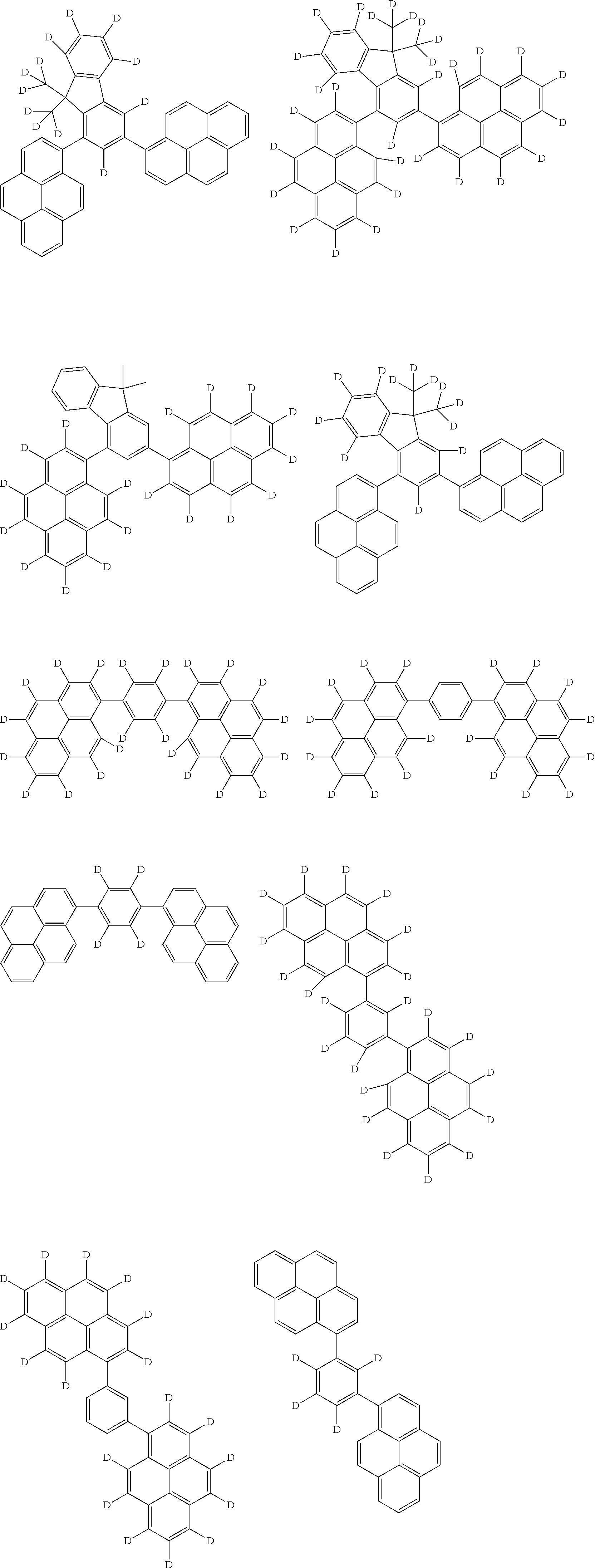

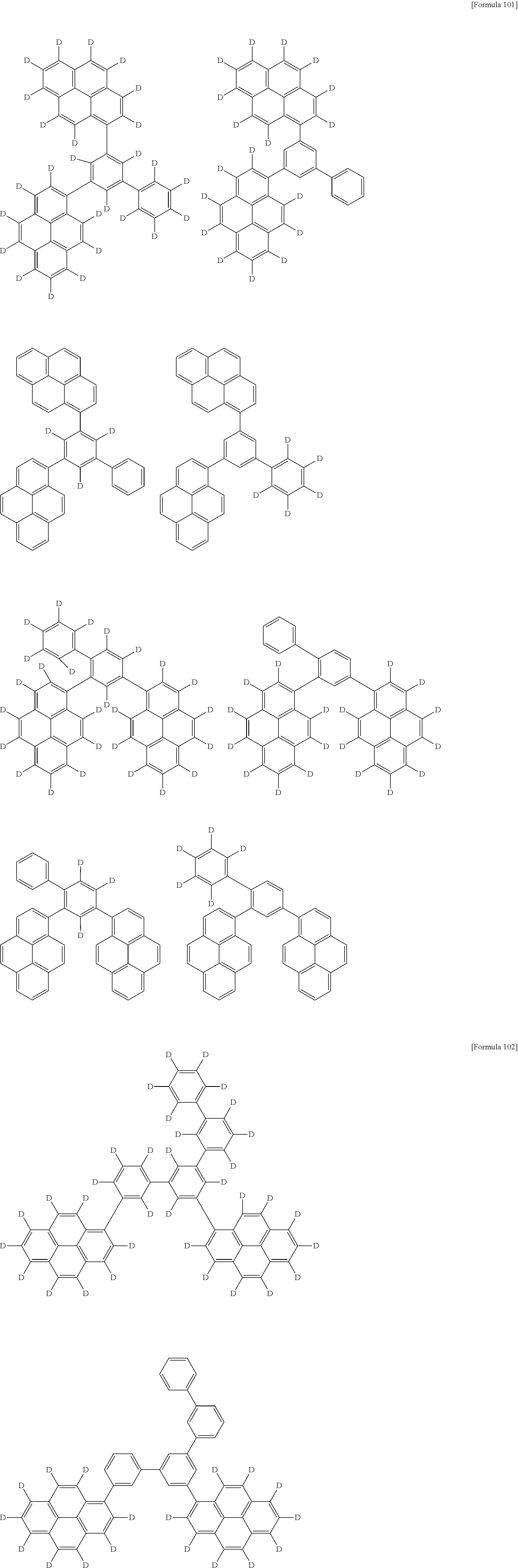

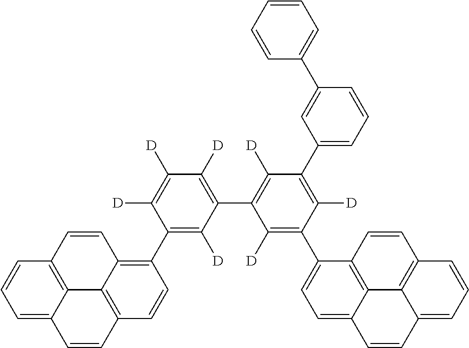 [SMILES: [2H]c1c([2H])c(-c2c([2H])c(-c3cccc(-c4ccccc4)c3)c([2H])c(-c3ccc4ccc5cccc6ccc3c4c56)c2[2H])c([2H])c(-c2ccc3ccc4cccc5ccc2c3c45)c1[2H]]

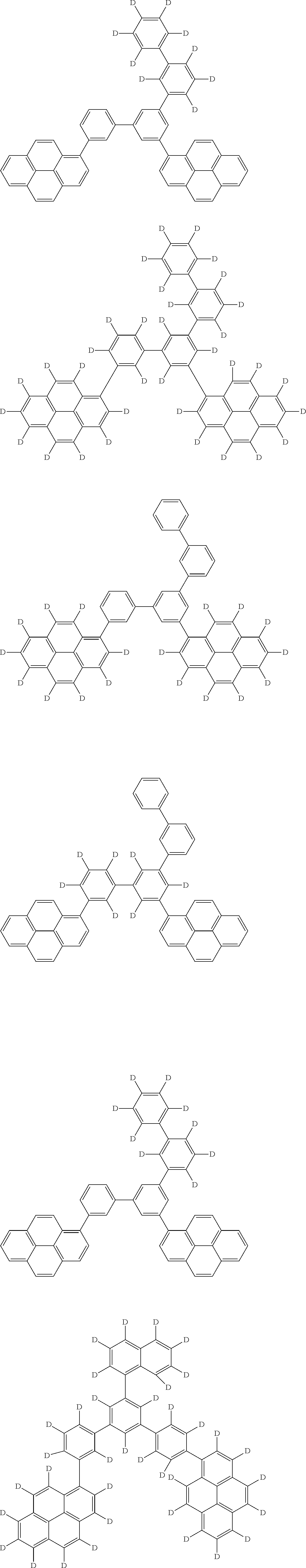

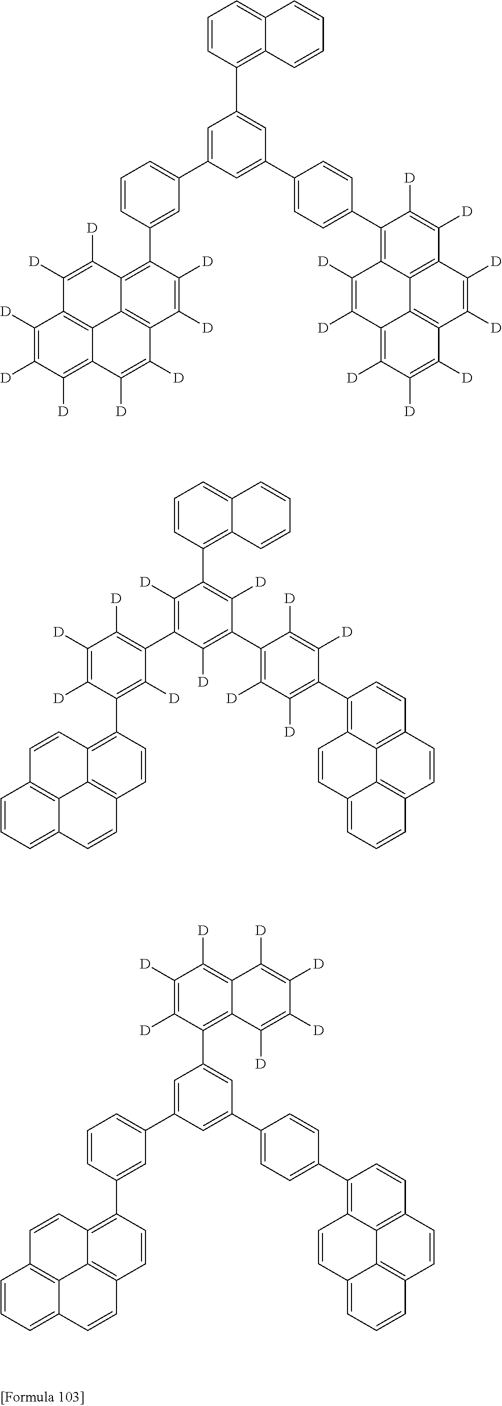

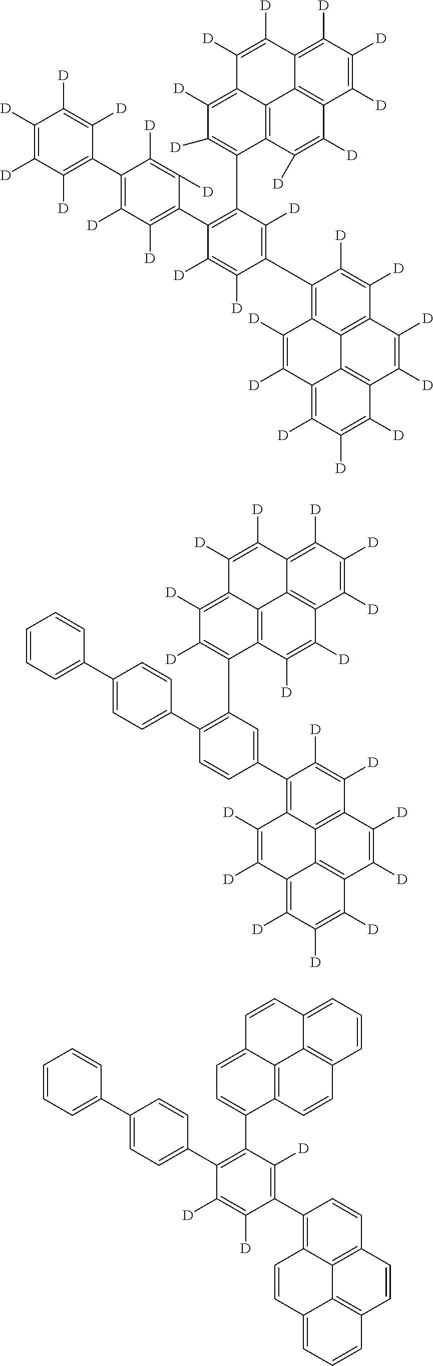

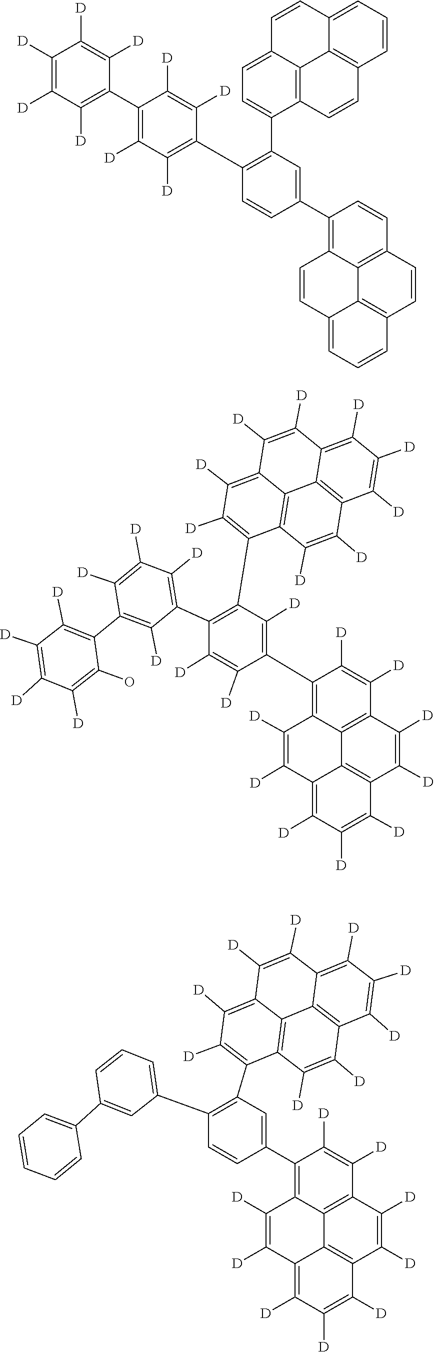

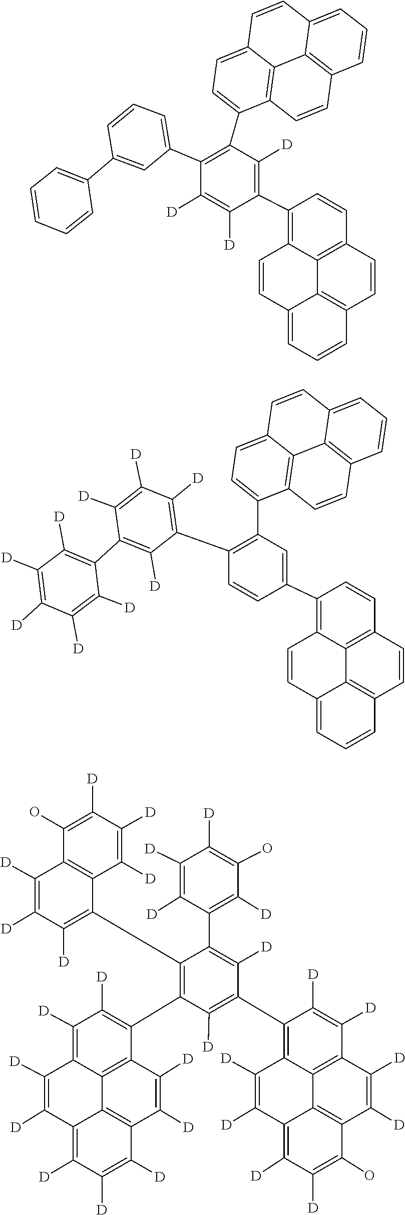

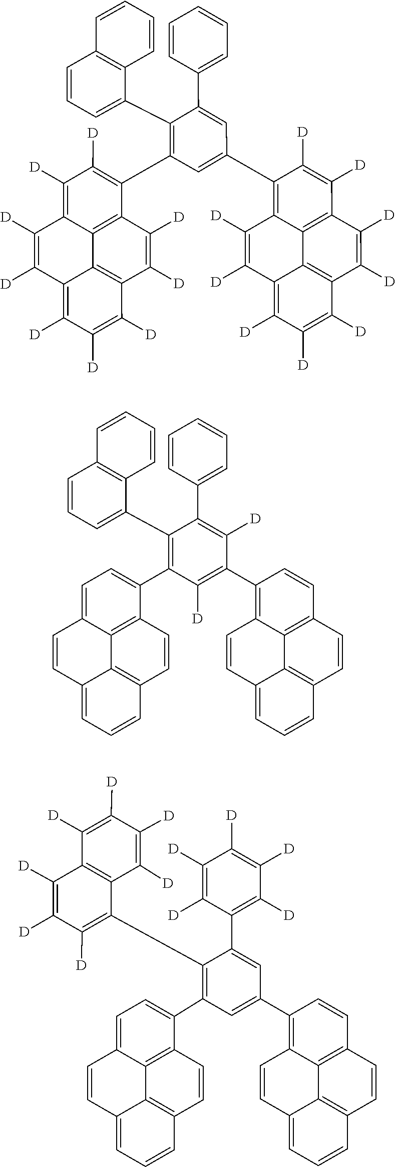

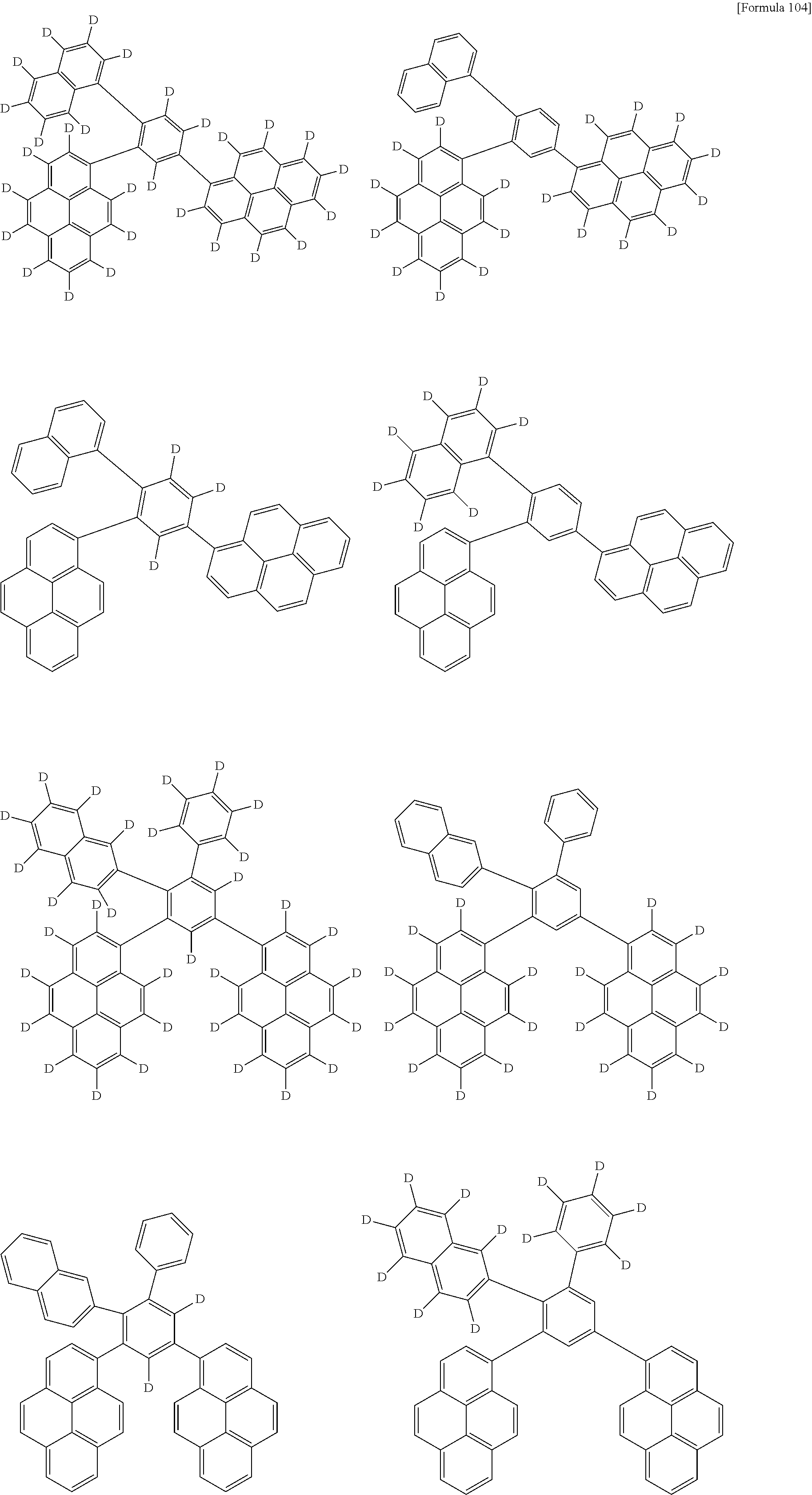

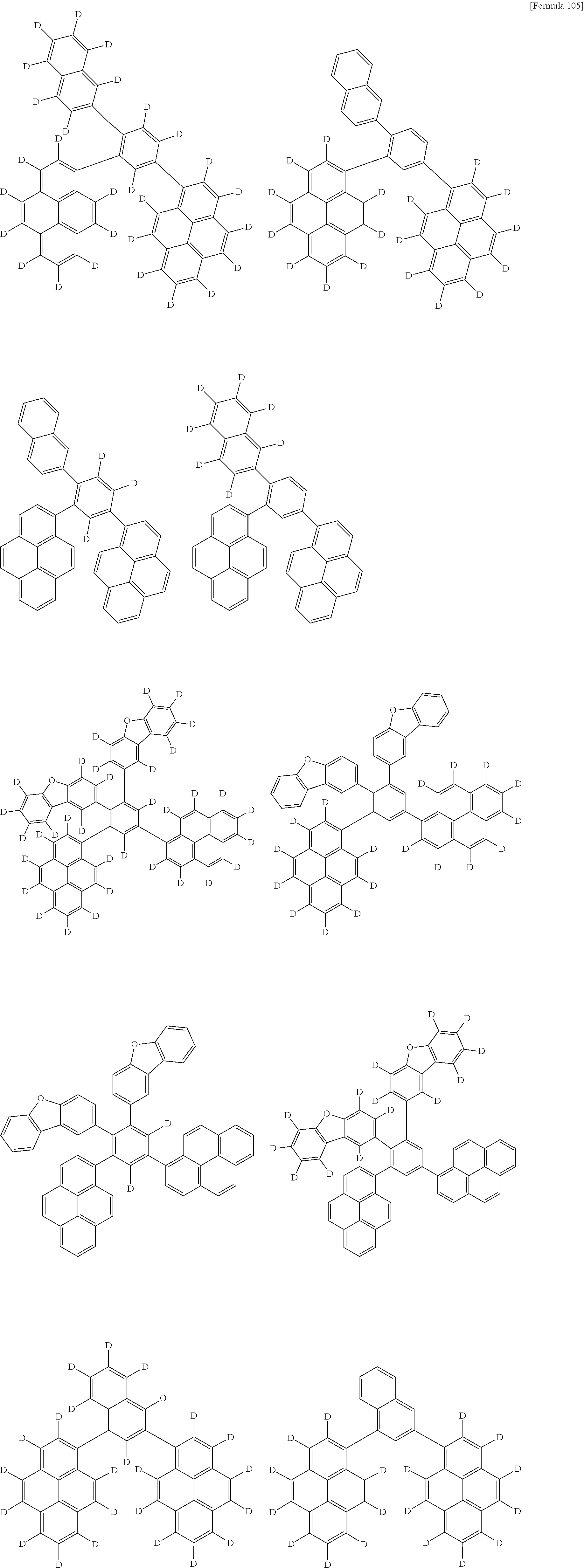

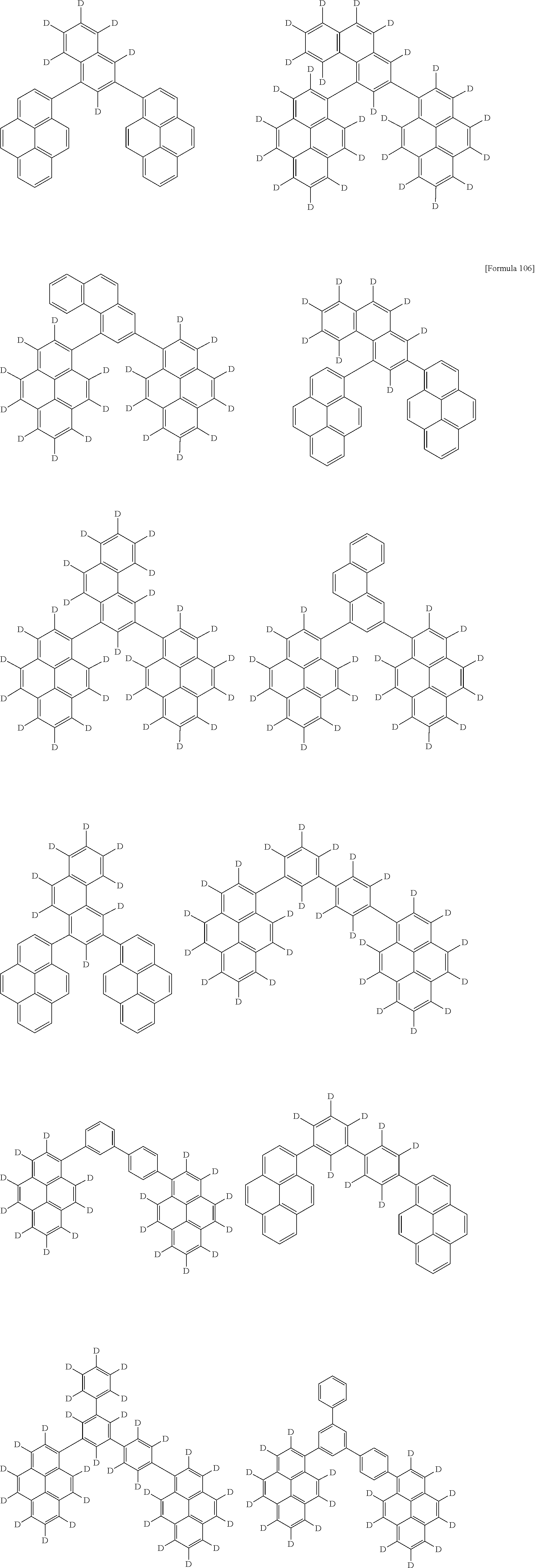

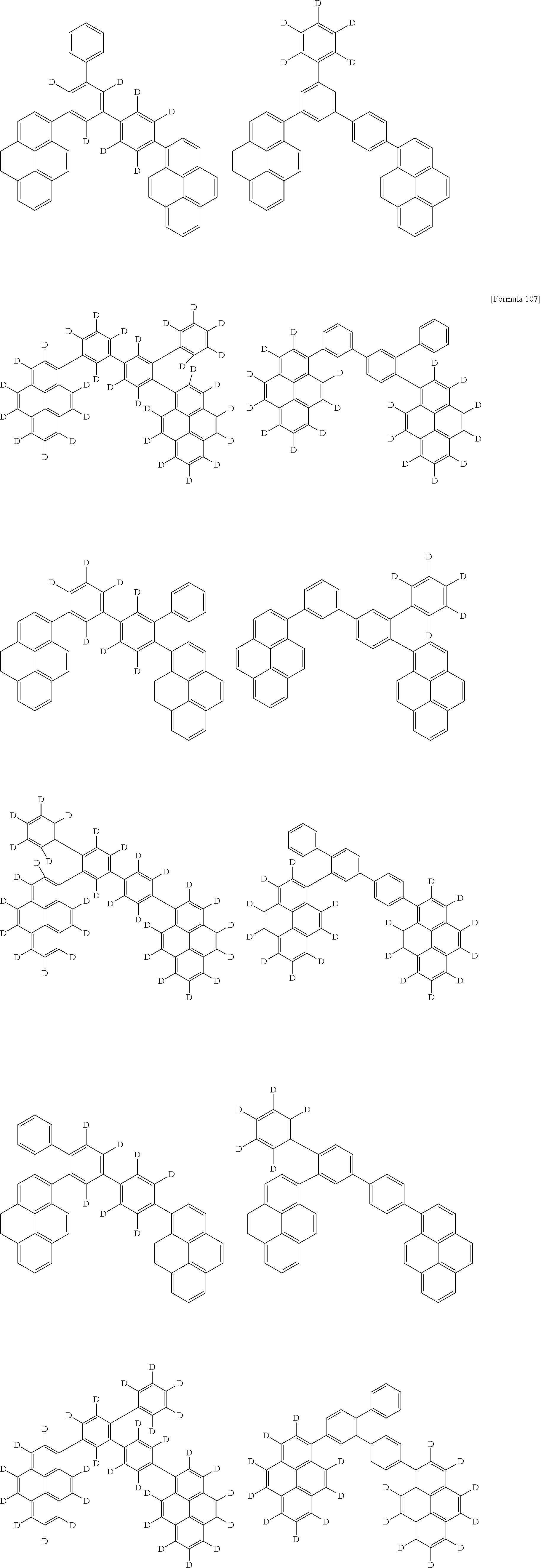

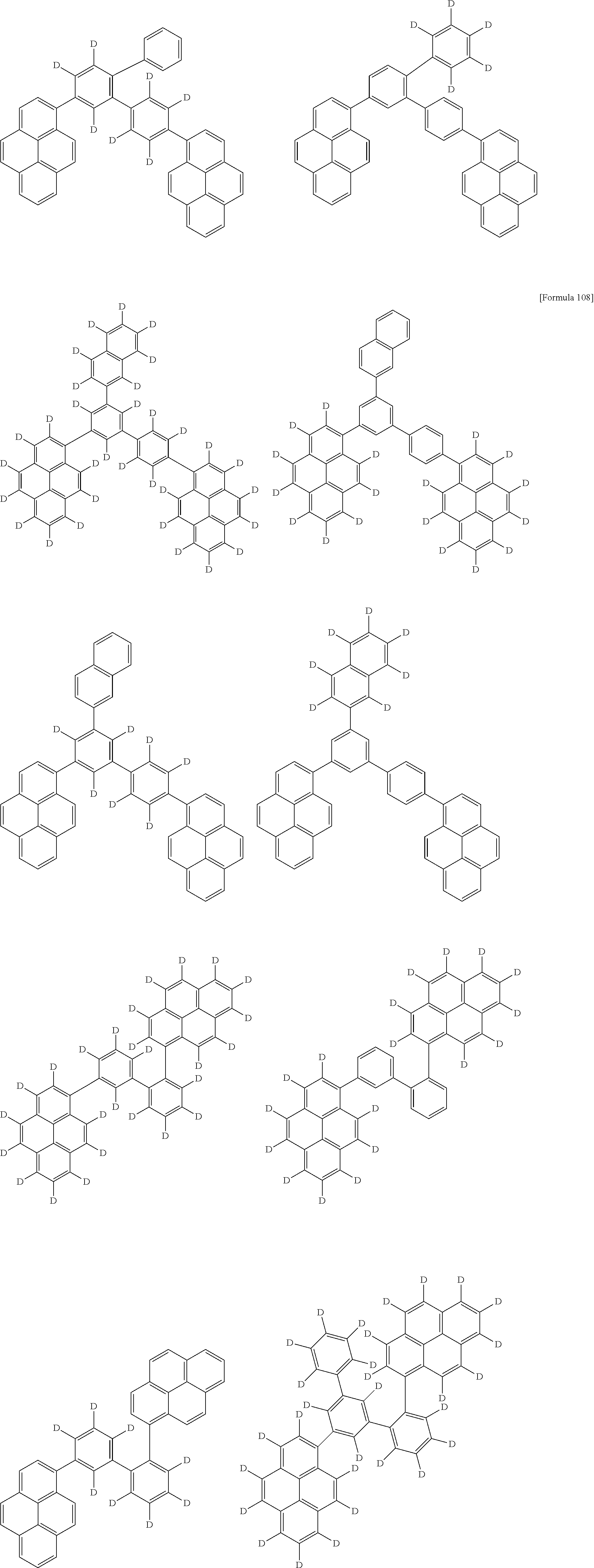

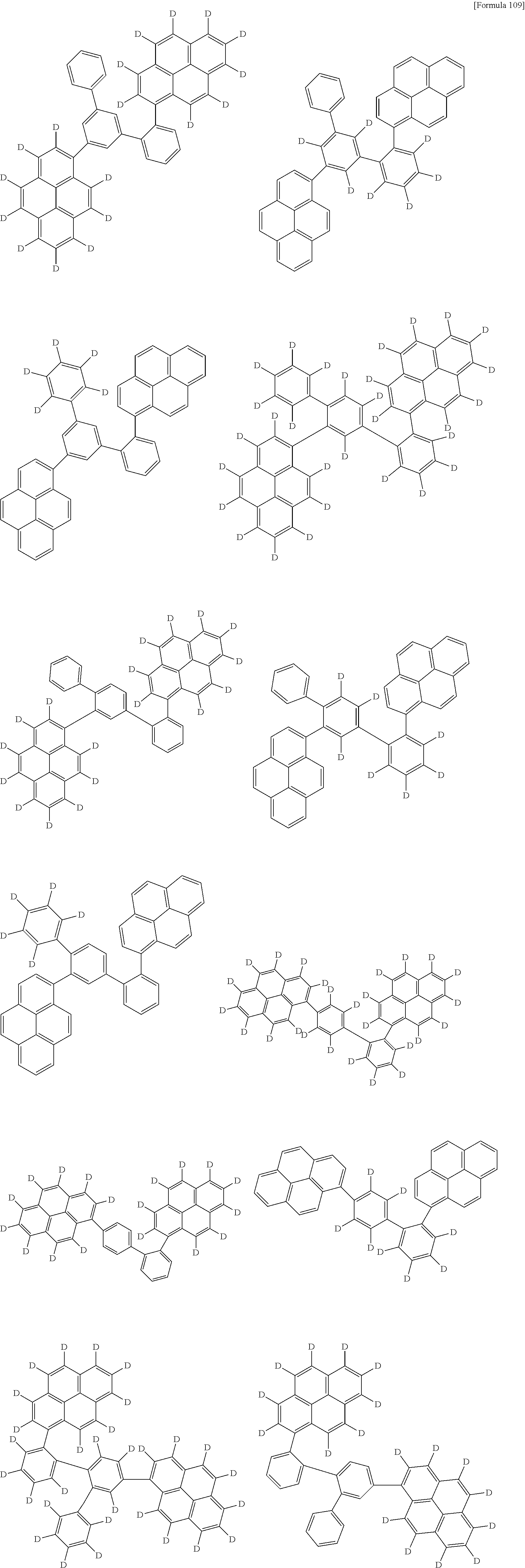

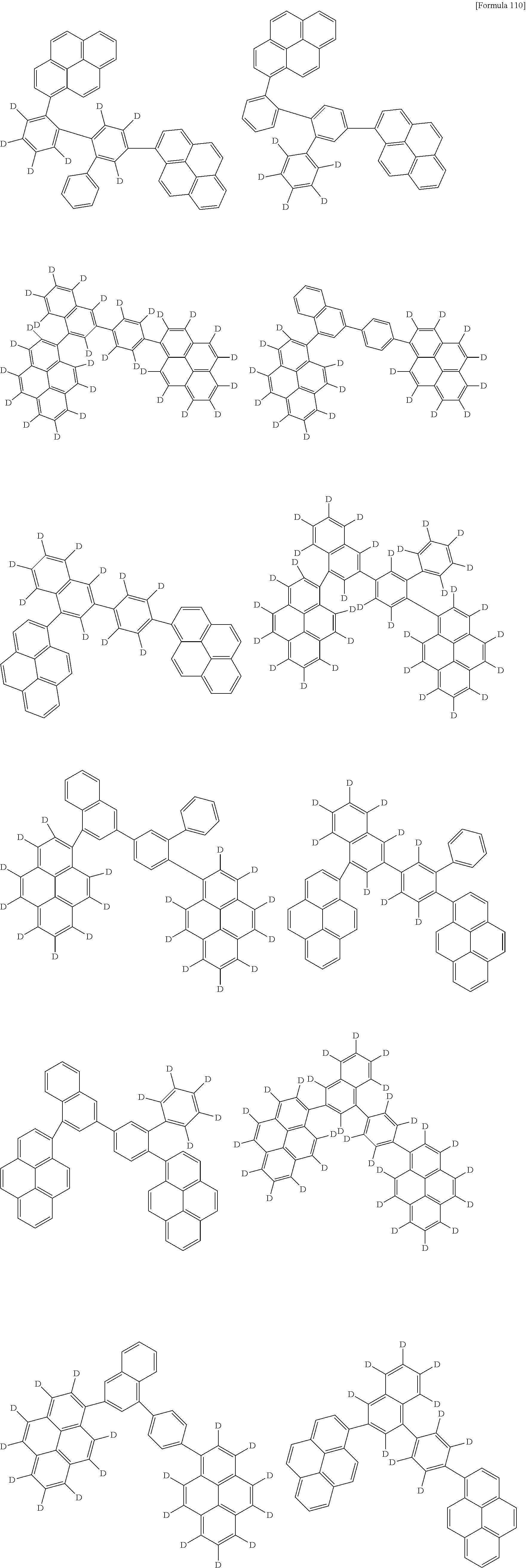

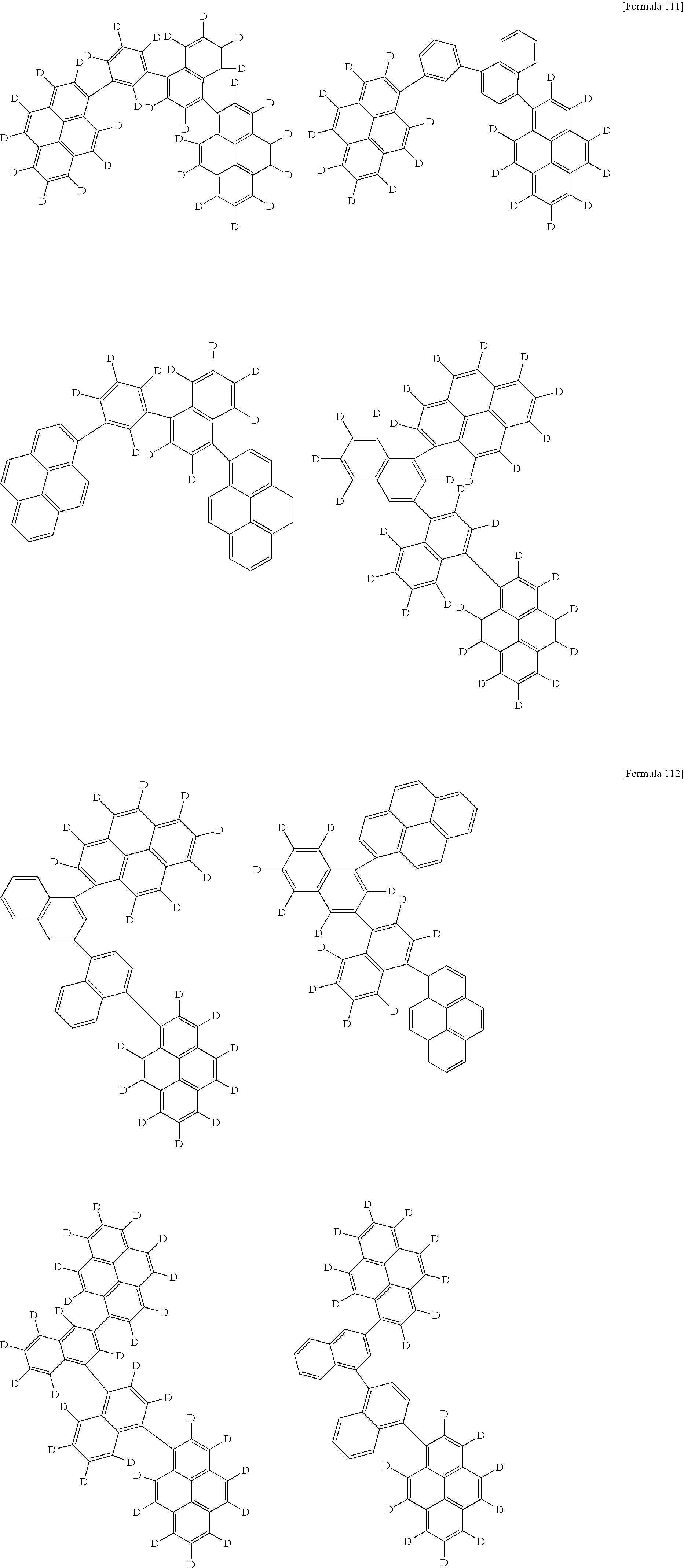

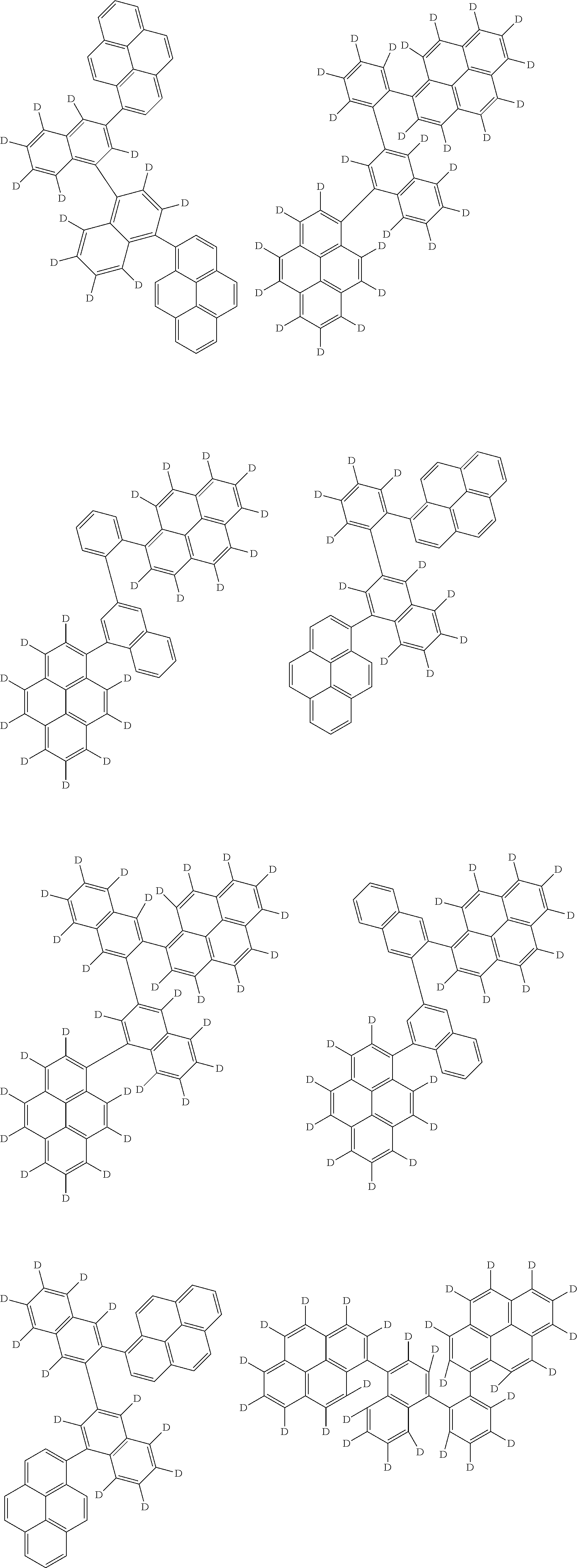

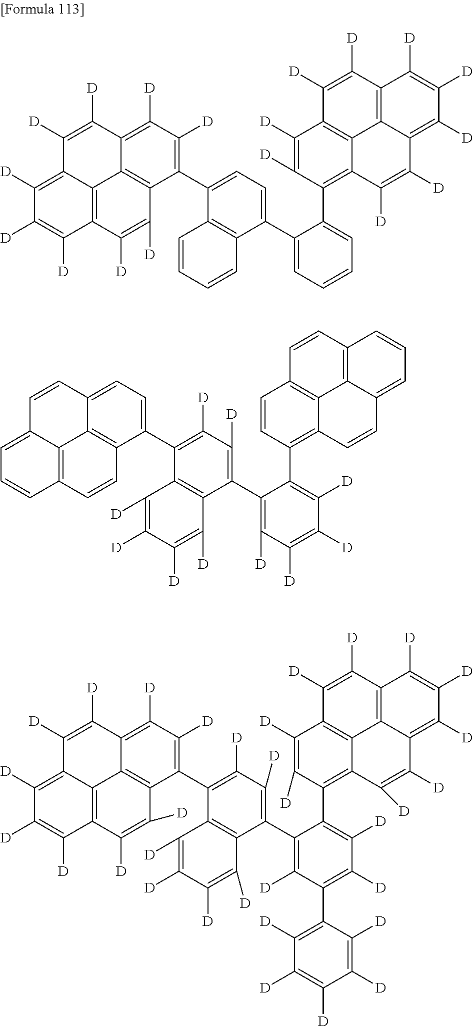

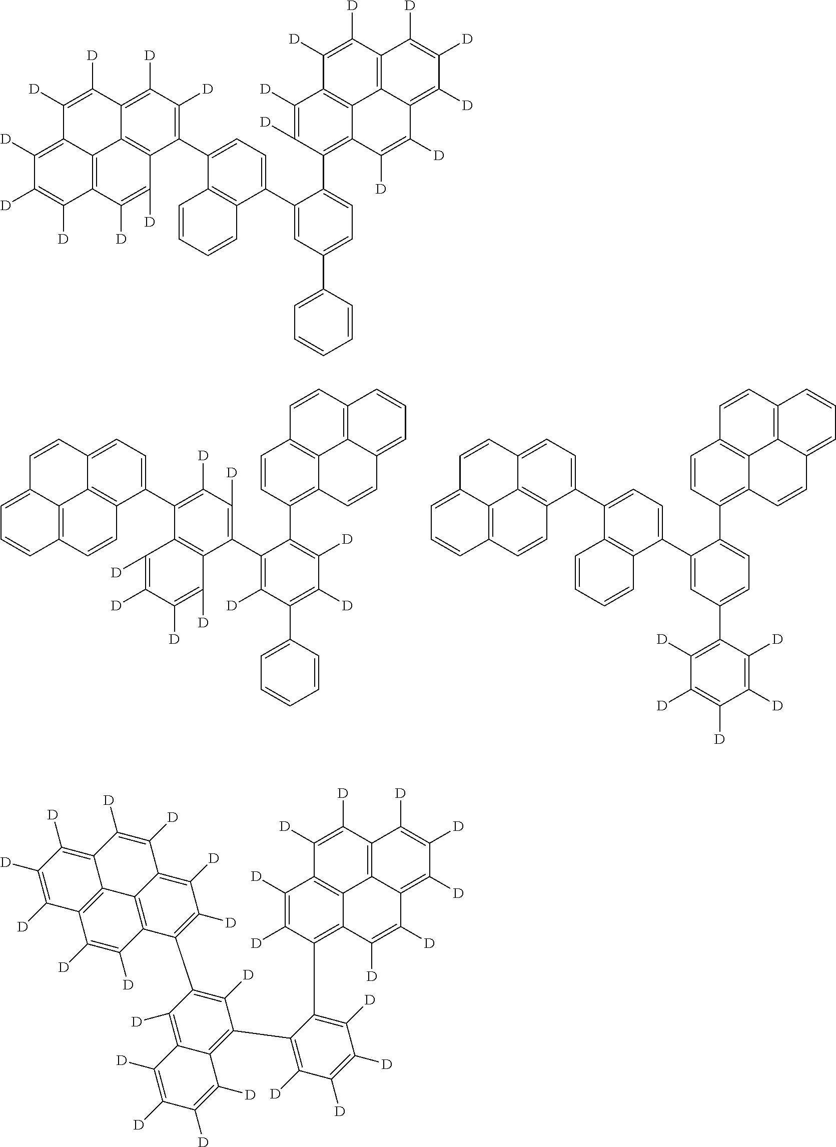

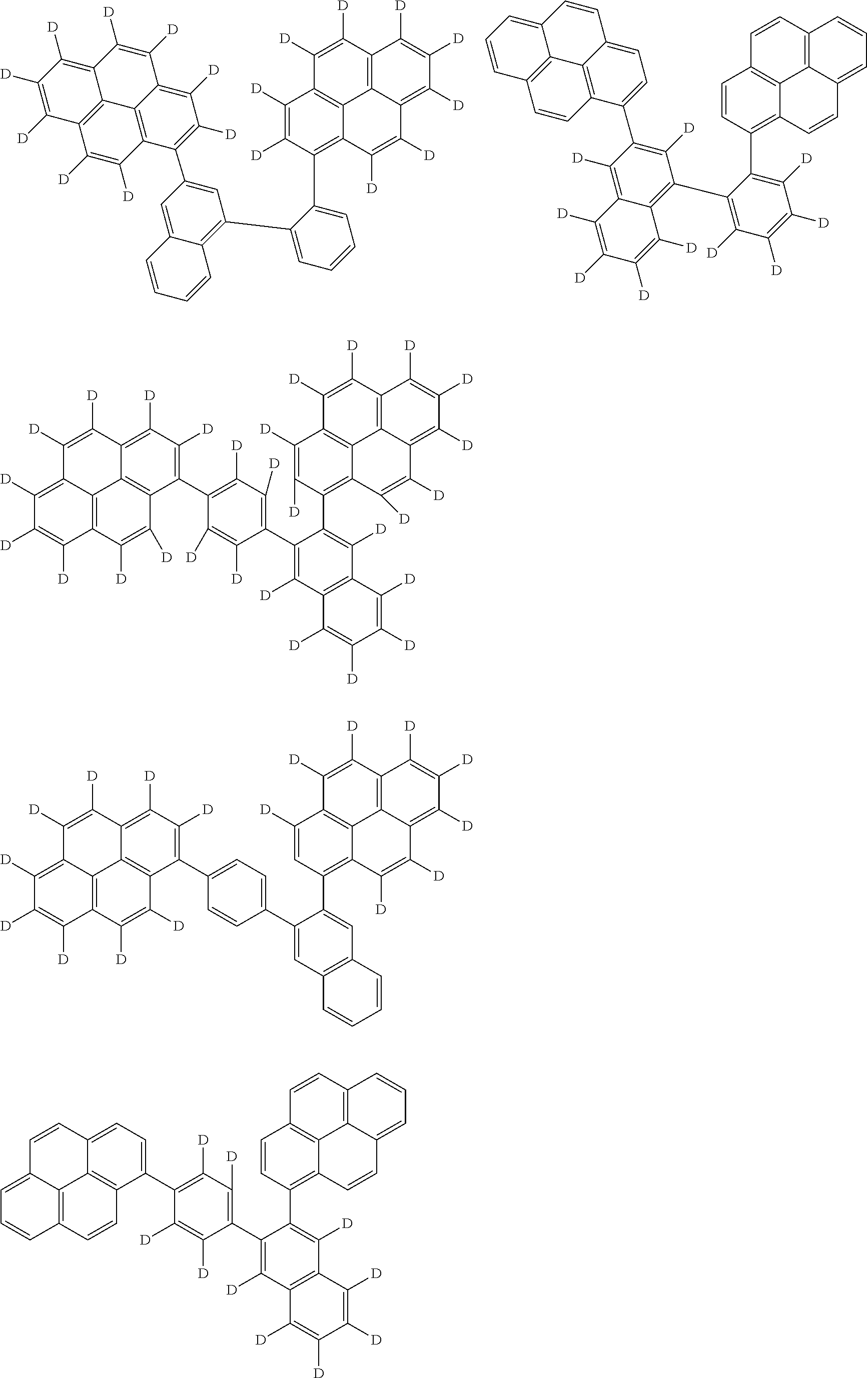

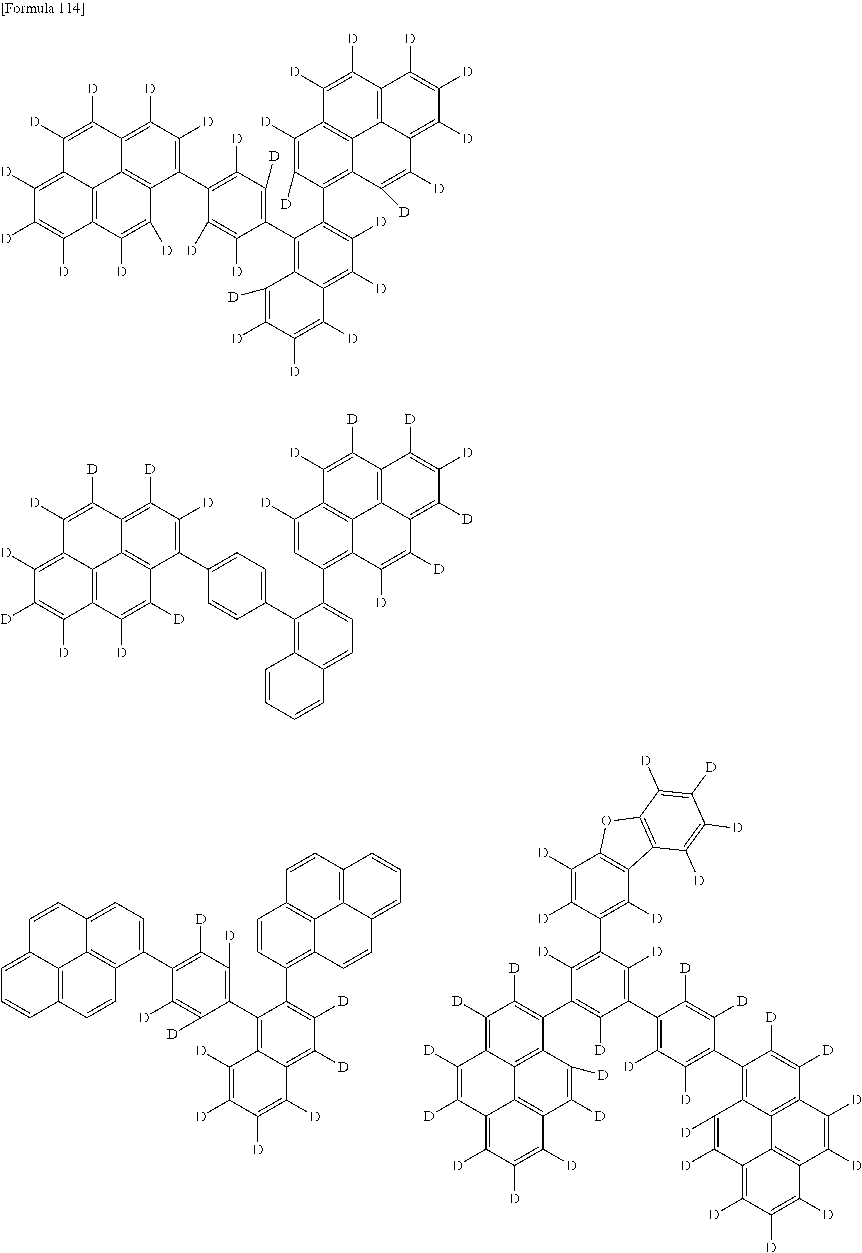

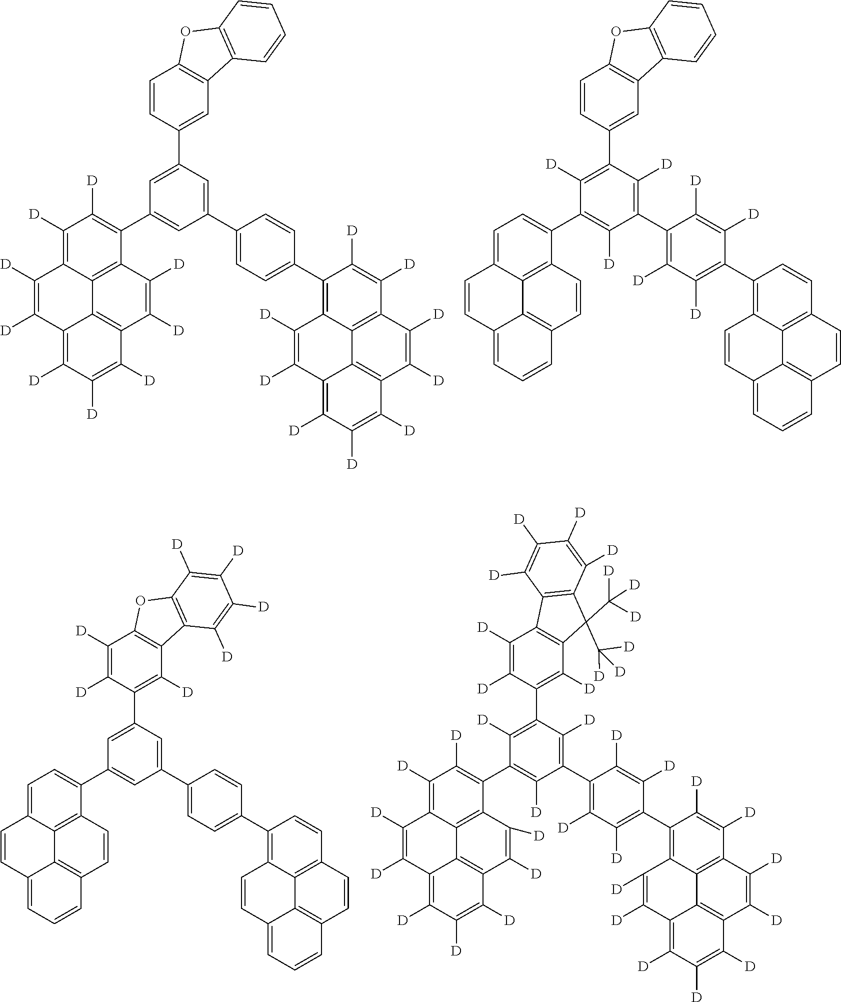

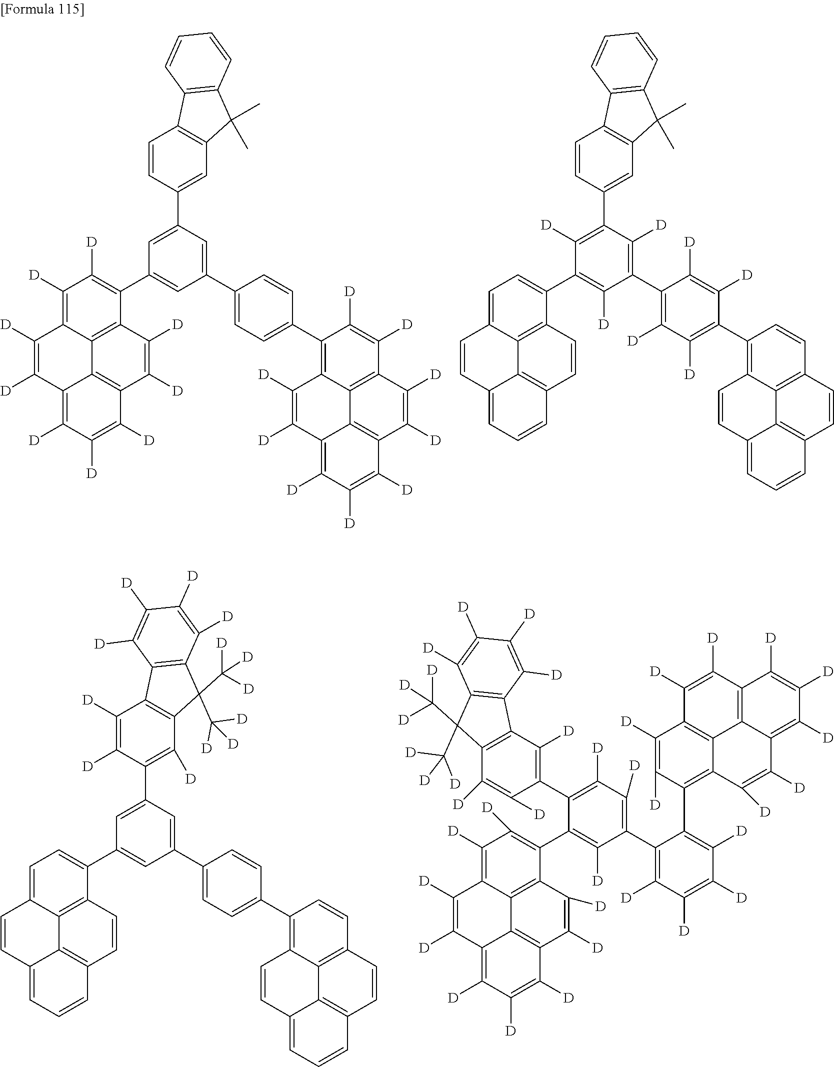

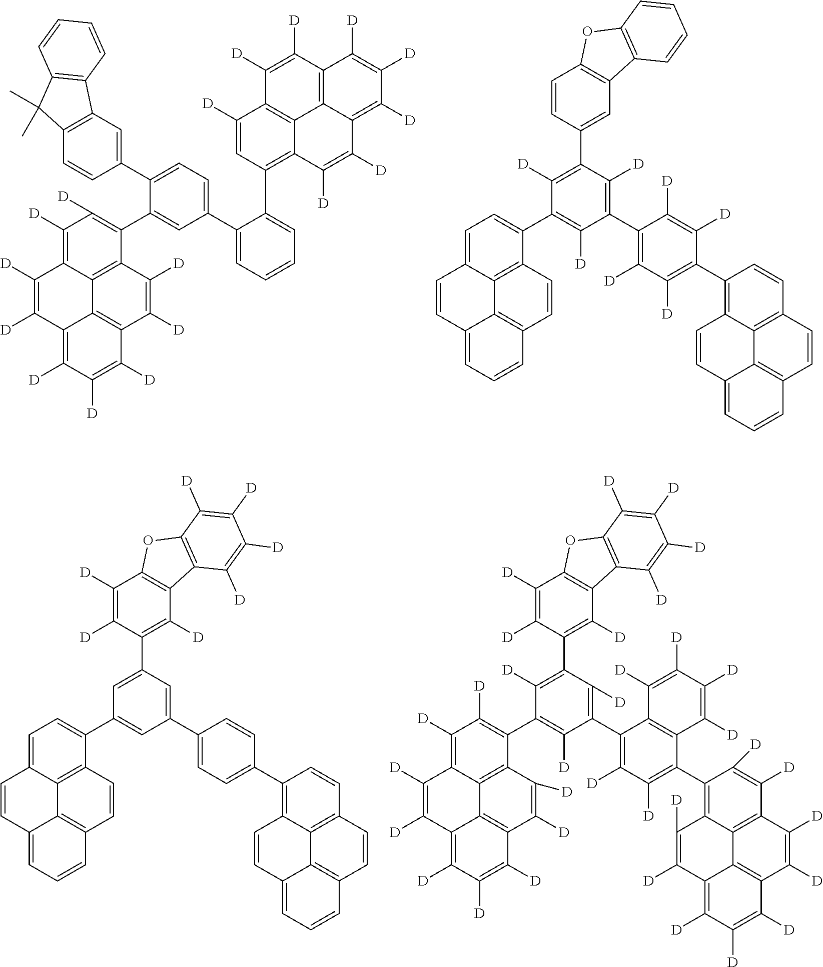

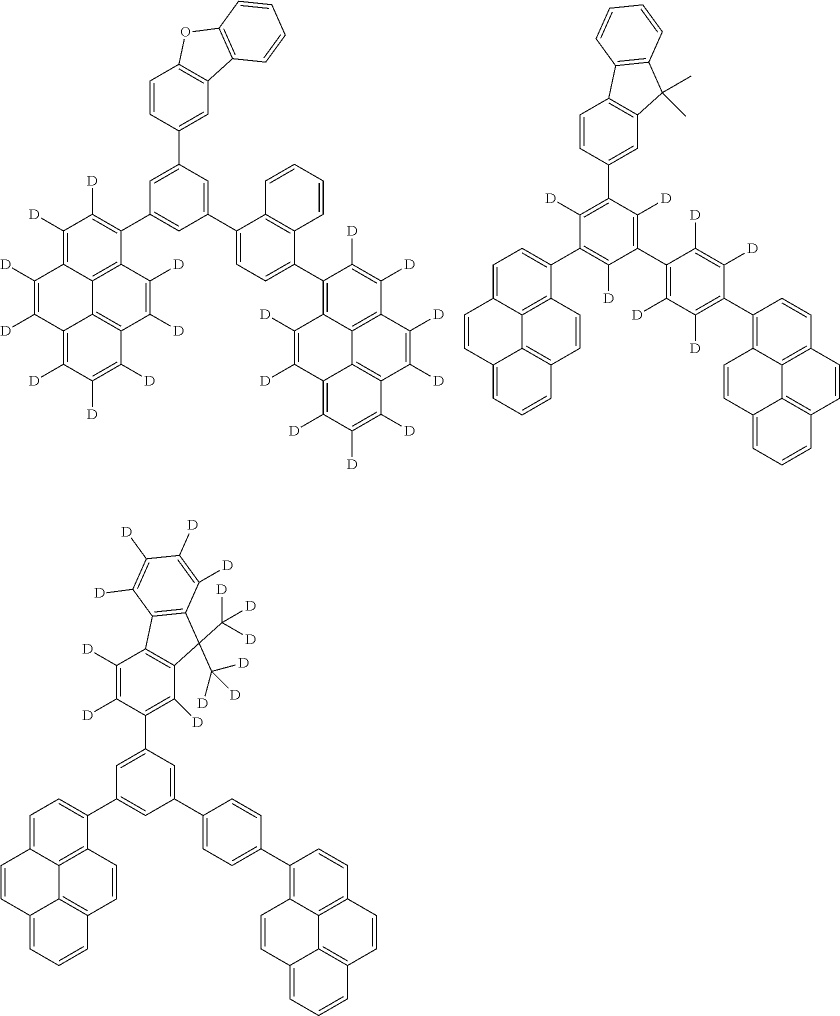

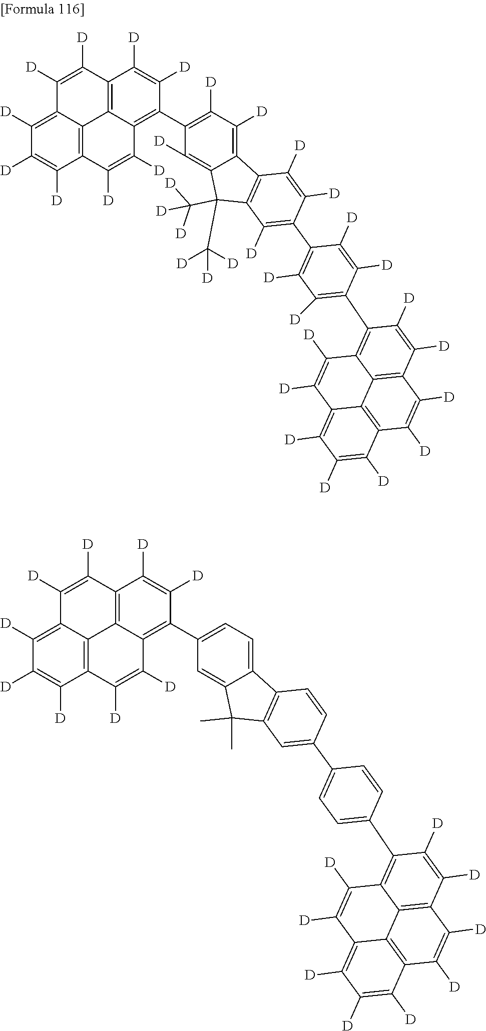 [SMILES: [2H]c1c([2H])c(-c2c([2H])c([2H])c3c([2H])c([2H])c4c([2H])c([2H])c([2H])c5c([2H])c([2H])c2c3c45)c([2H])c([2H])c1-c1c([2H])c([2H])c2c(c1[2H])C(C([2H])([2H])[2H])(C([2H])([2H])[2H])c1c([2H])c(-c3c([2H])c([2H])c4c([2H])c([2H])c5c([2H])c([2H])c([2H])c6c([2H])c([2H])c3c4c56)c([2H])c([2H])c1-2 ; [2H]c1c([2H])c2c([2H])c([2H])c3c([2H])c([2H])c(-c4ccc(-c5ccc6c(c5)C(C)(C)c5cc(-c7c([2H])c([2H])c8c([2H])c([2H])c9c([2H])c([2H])c([2H])c%10c([2H])c([2H])c7c8c9%10)ccc5-6)cc4)c4c([2H])c([2H])c(c1[2H])c2c34]

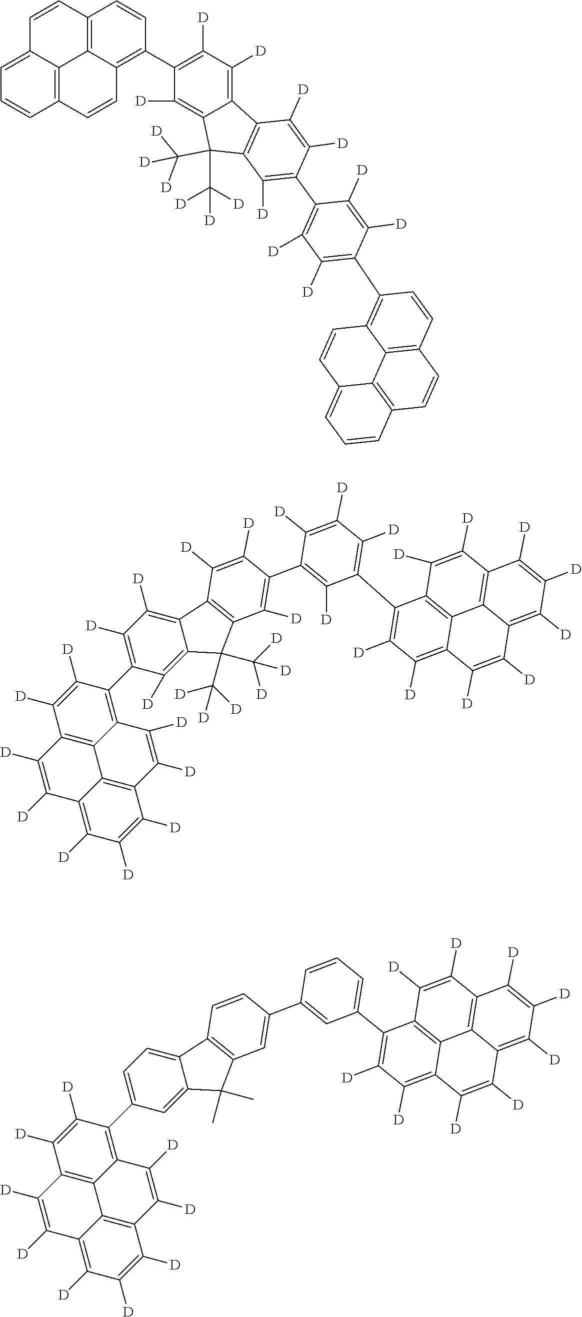

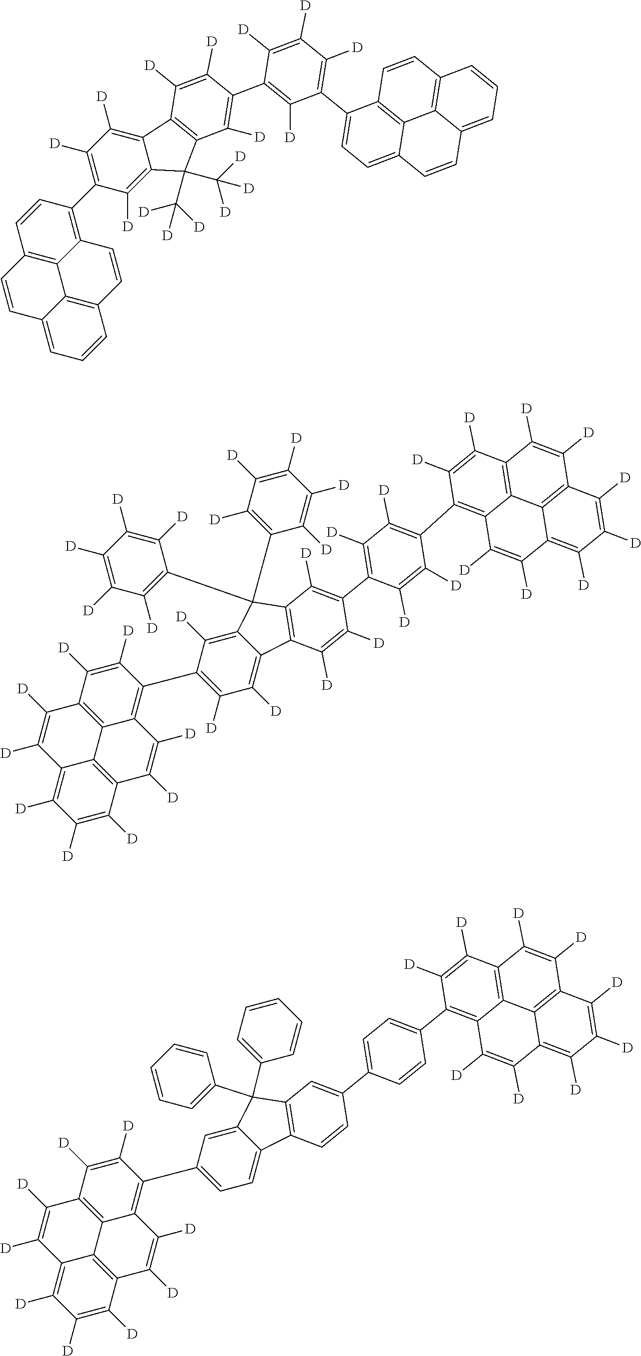

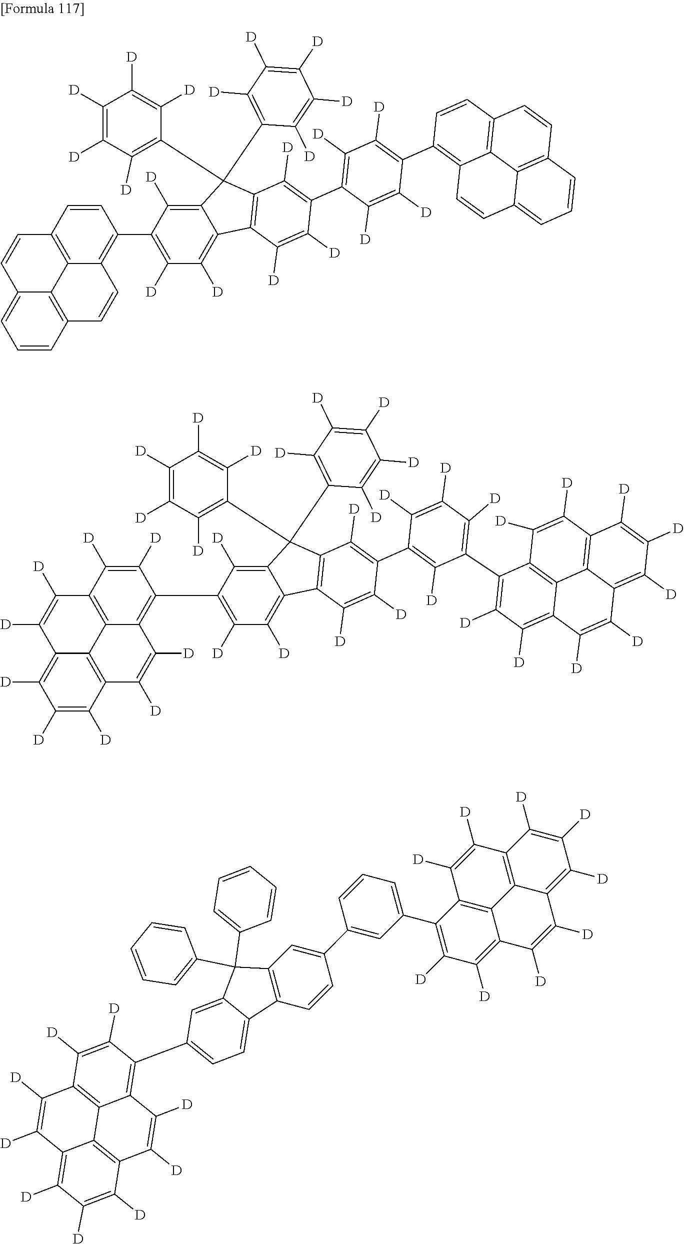

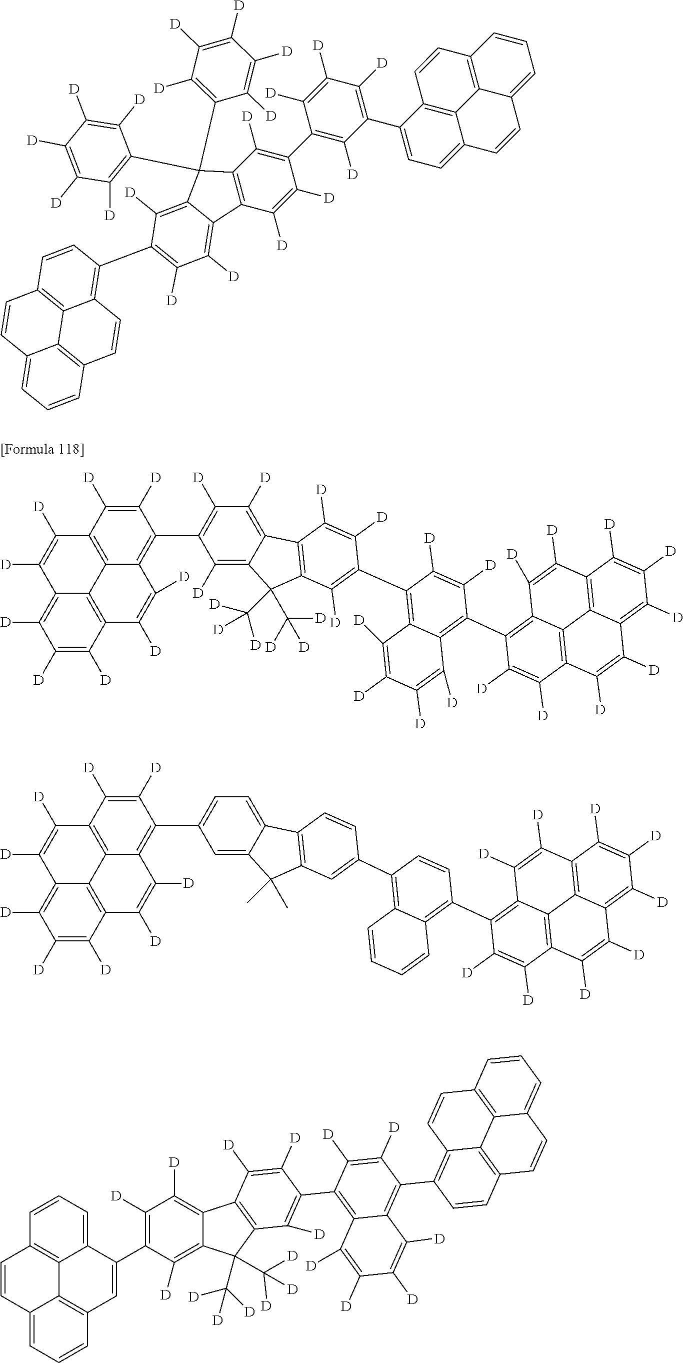

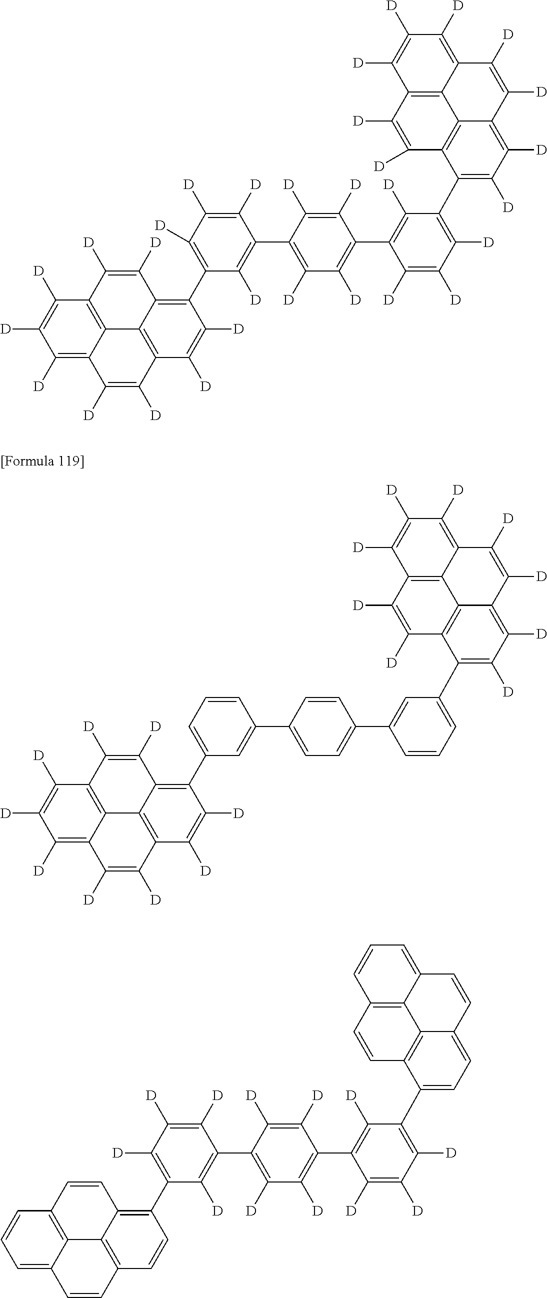

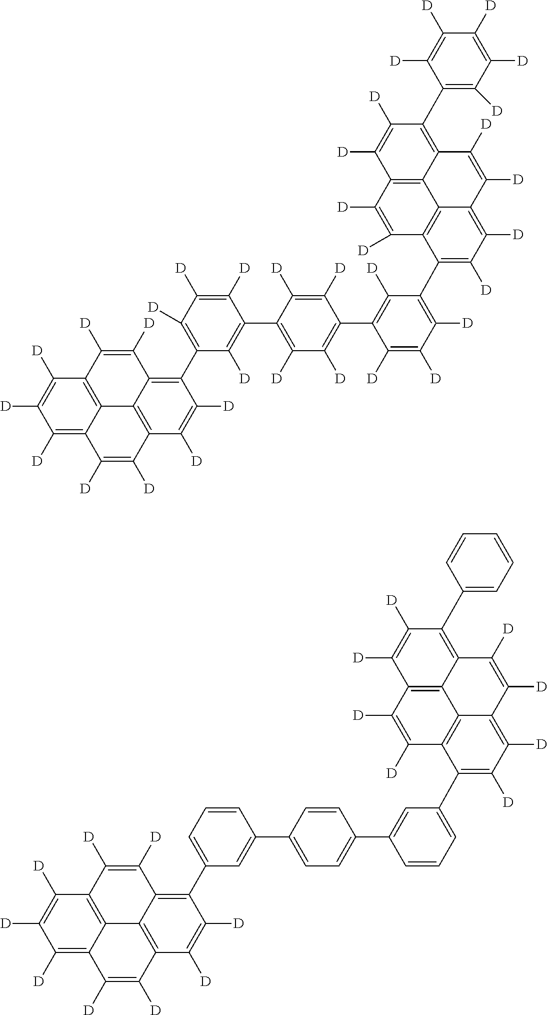

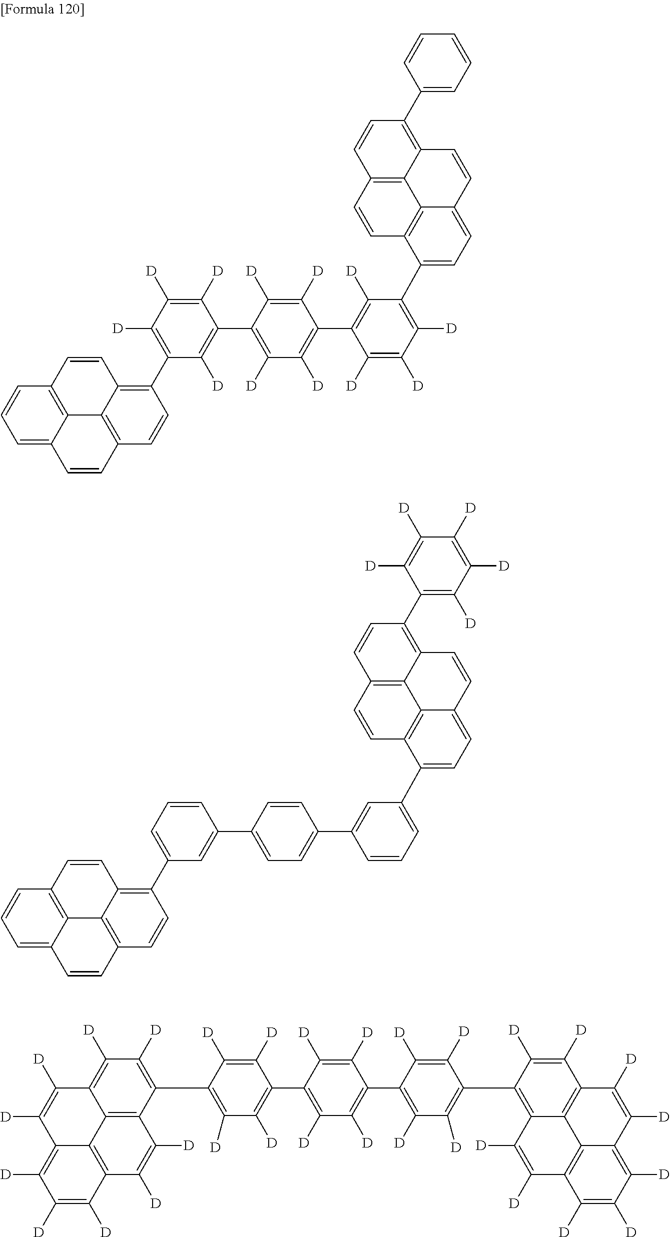

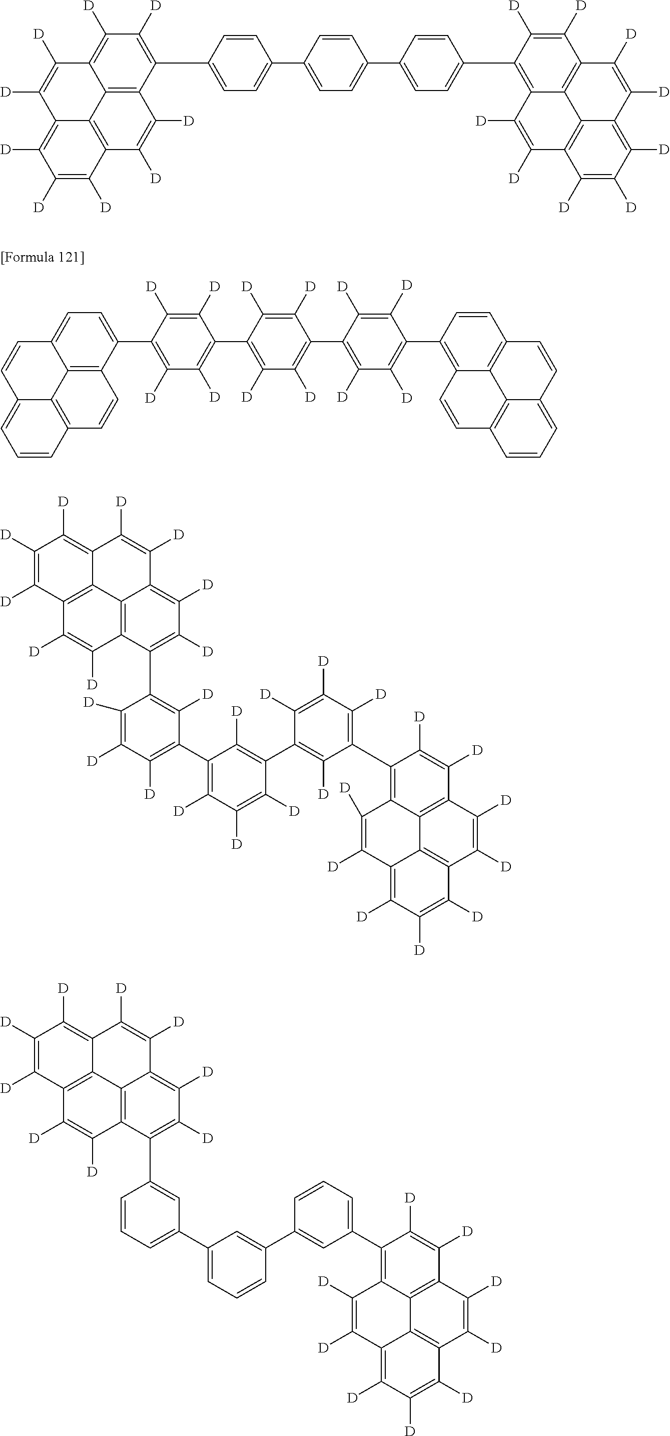

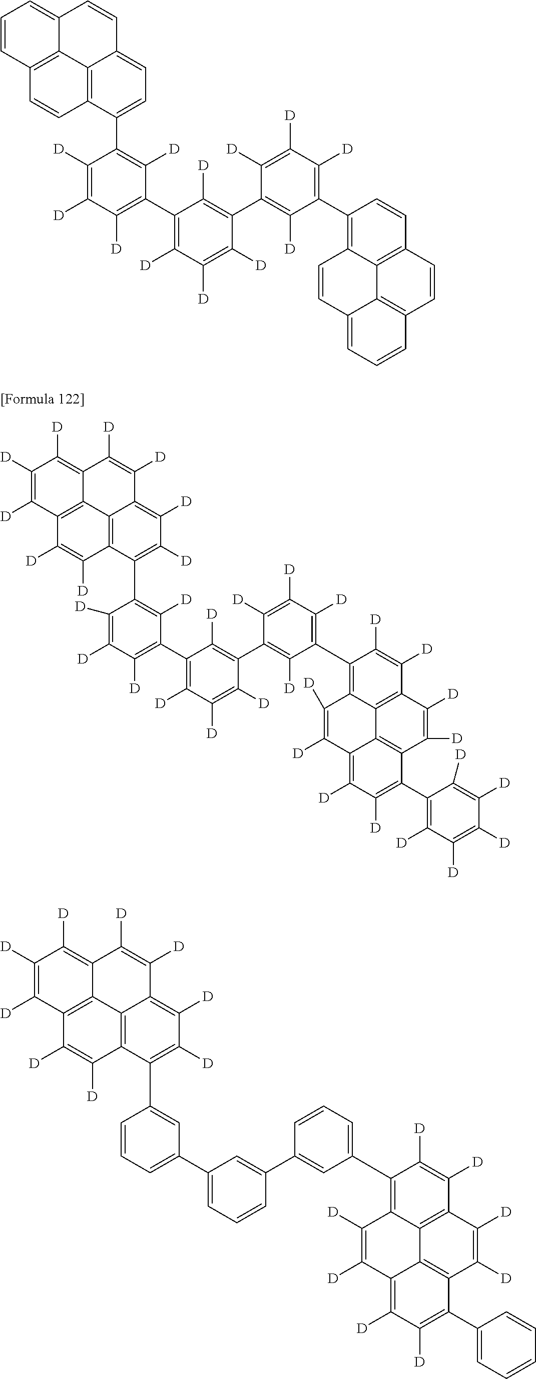

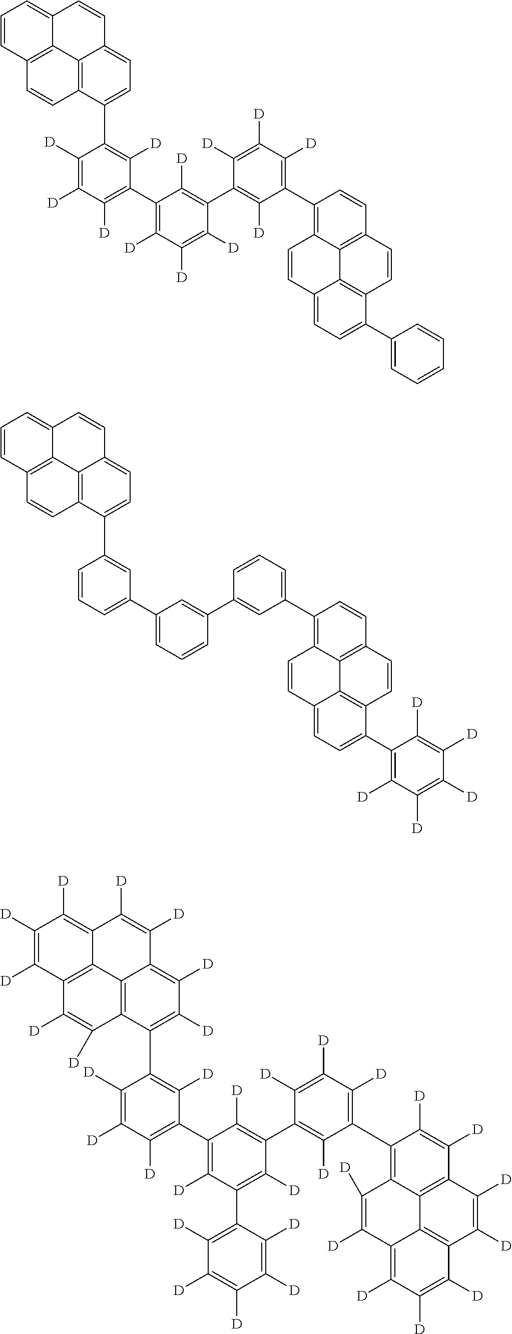

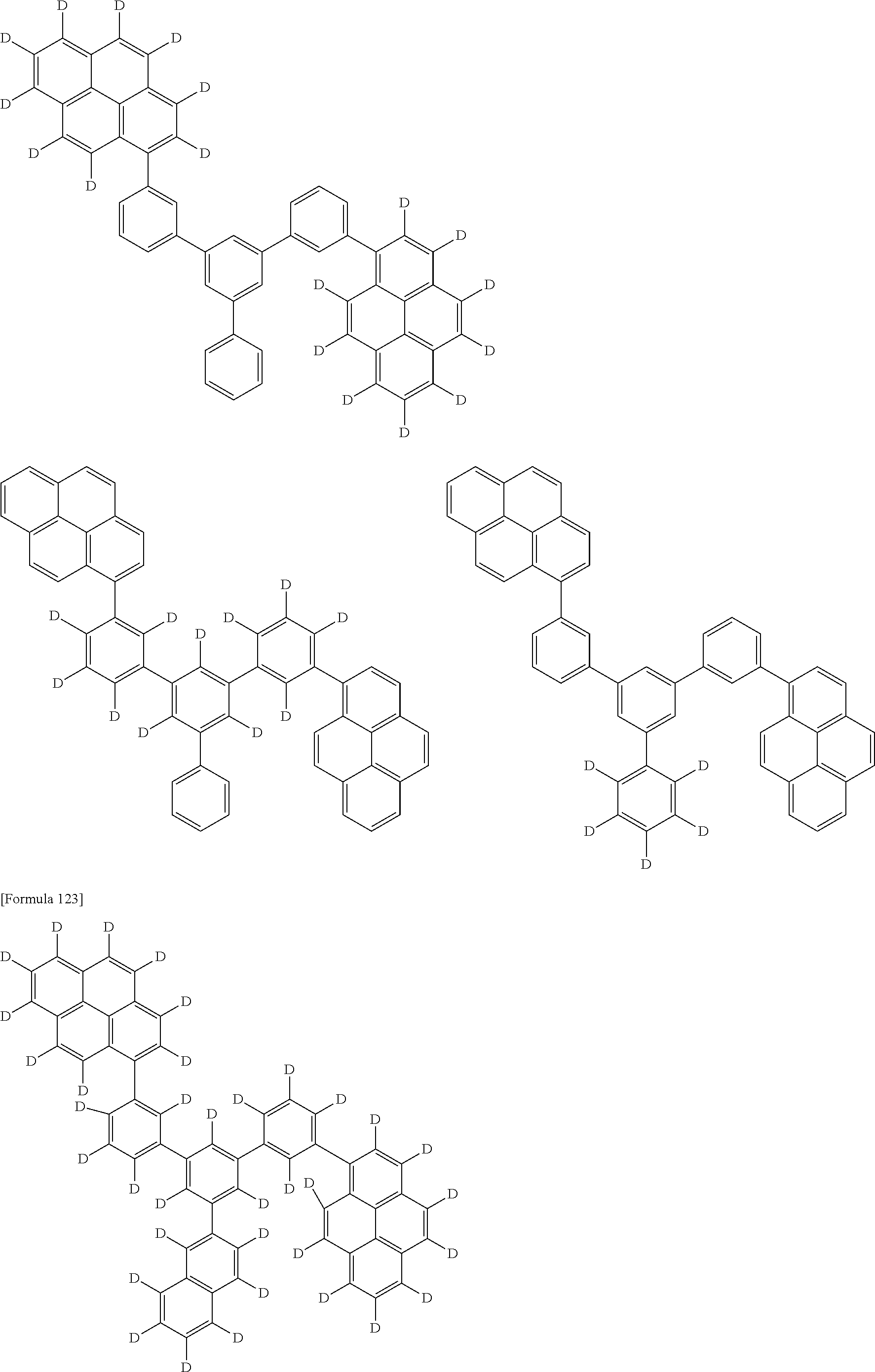

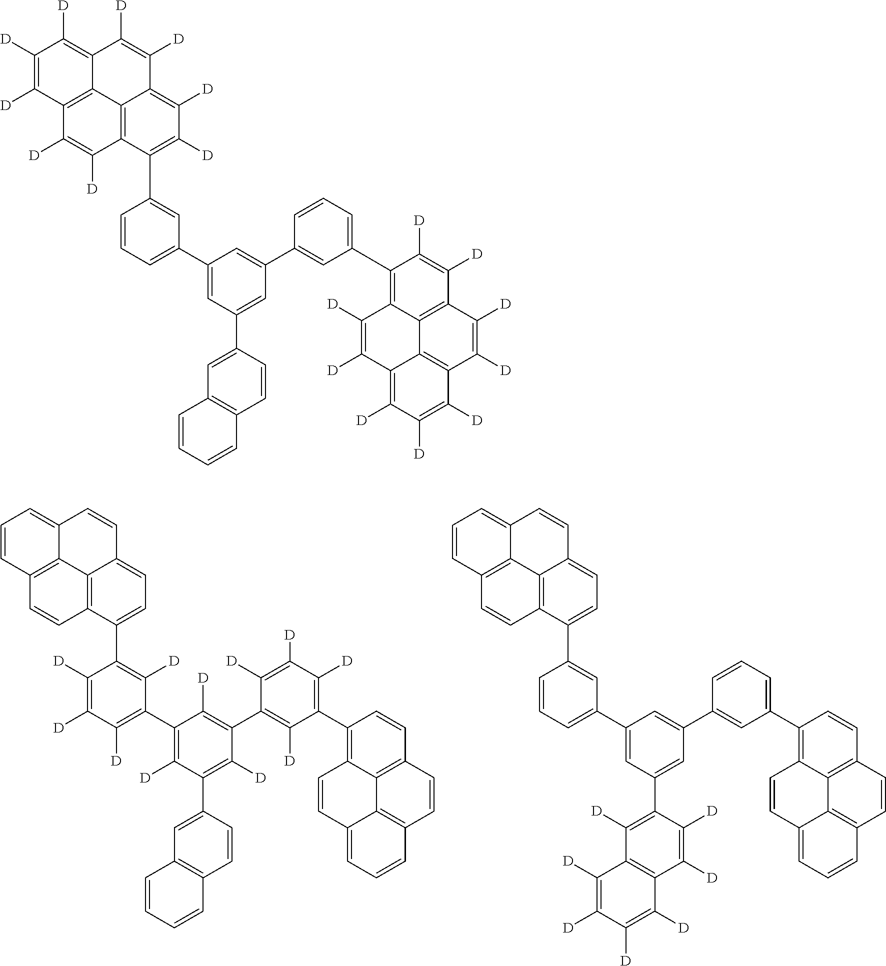

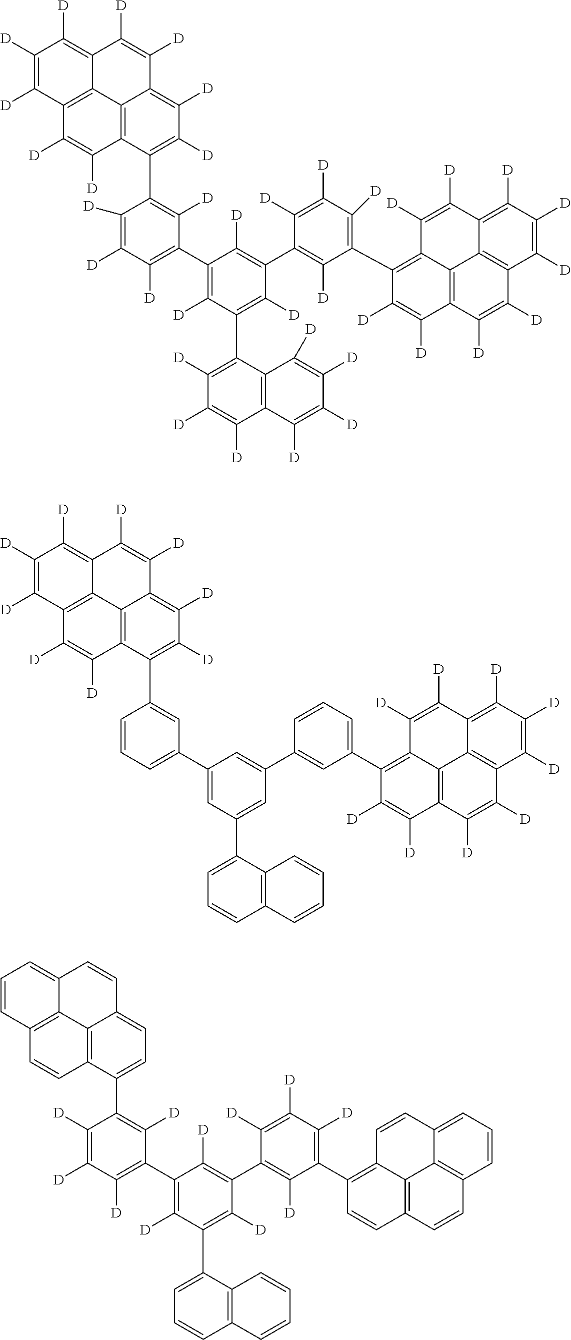

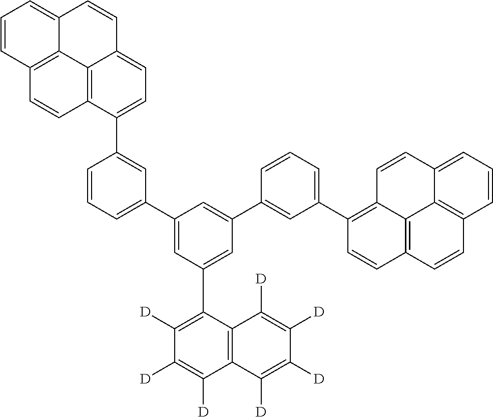 [SMILES: [2H]c1c([2H])c([2H])c2c(-c3cc(-c4cccc(-c5ccc6ccc7cccc8ccc5c6c78)c4)cc(-c4cccc(-c5ccc6ccc7cccc8ccc5c6c78)c4)c3)c([2H])c([2H])c([2H])c2c1[2H]]

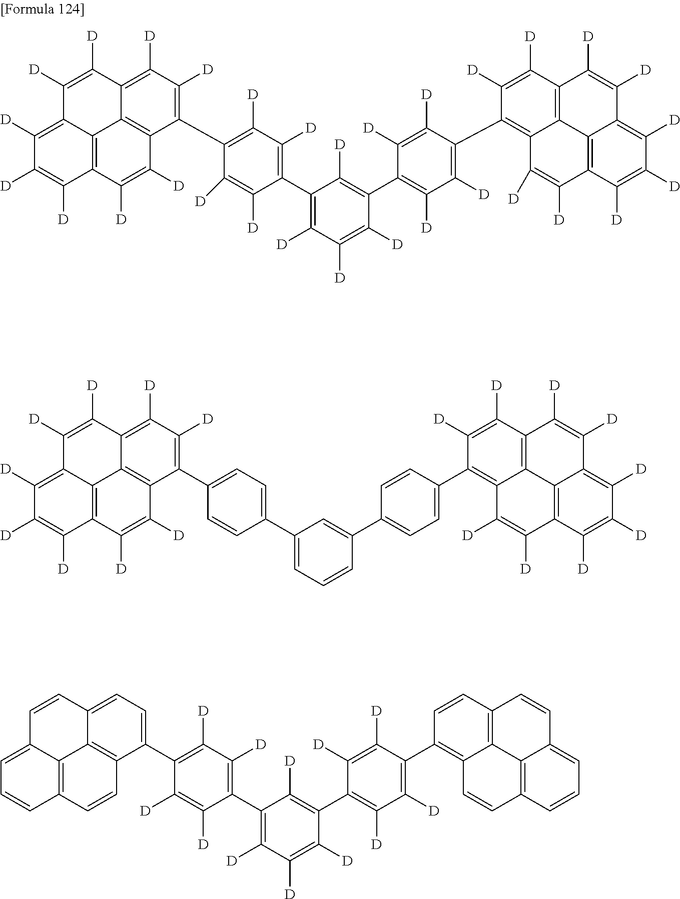

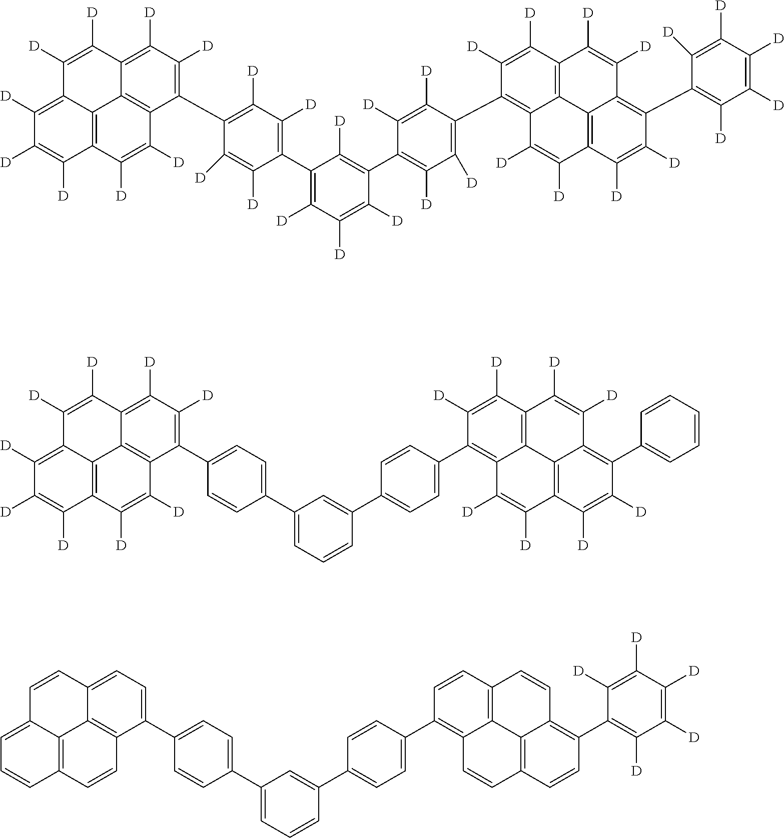

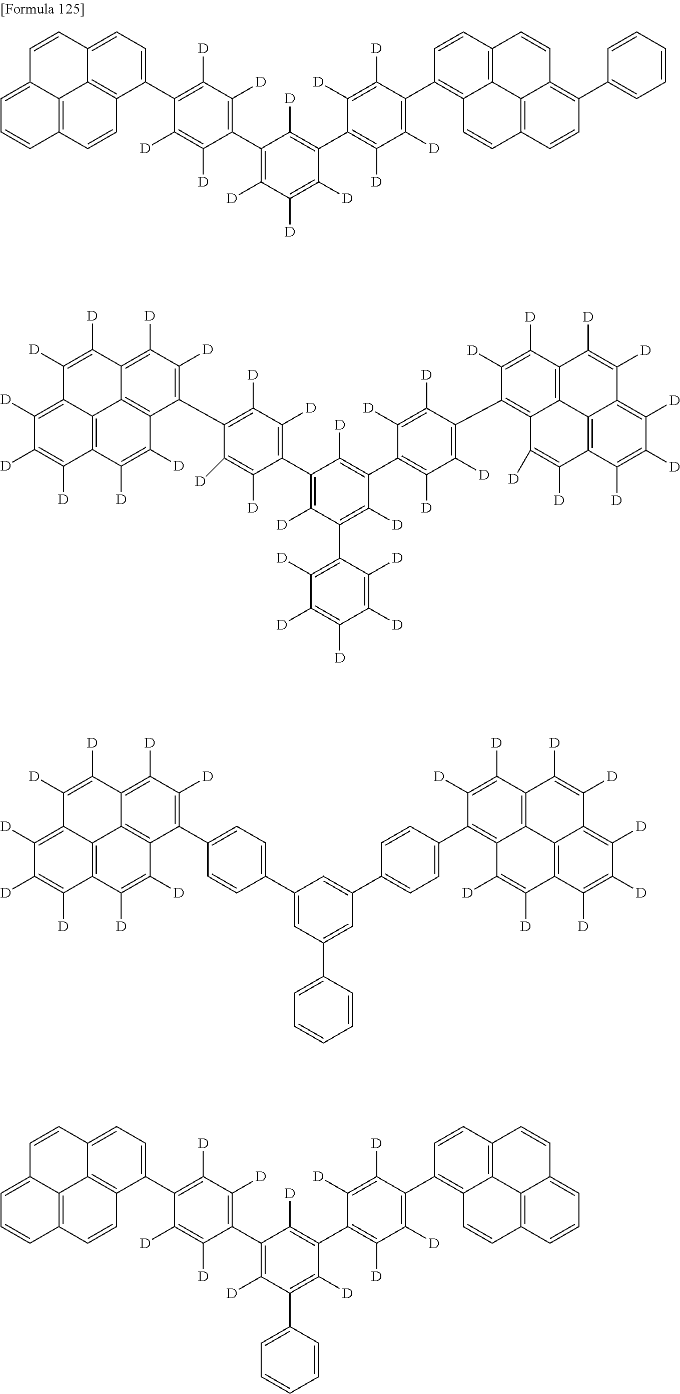

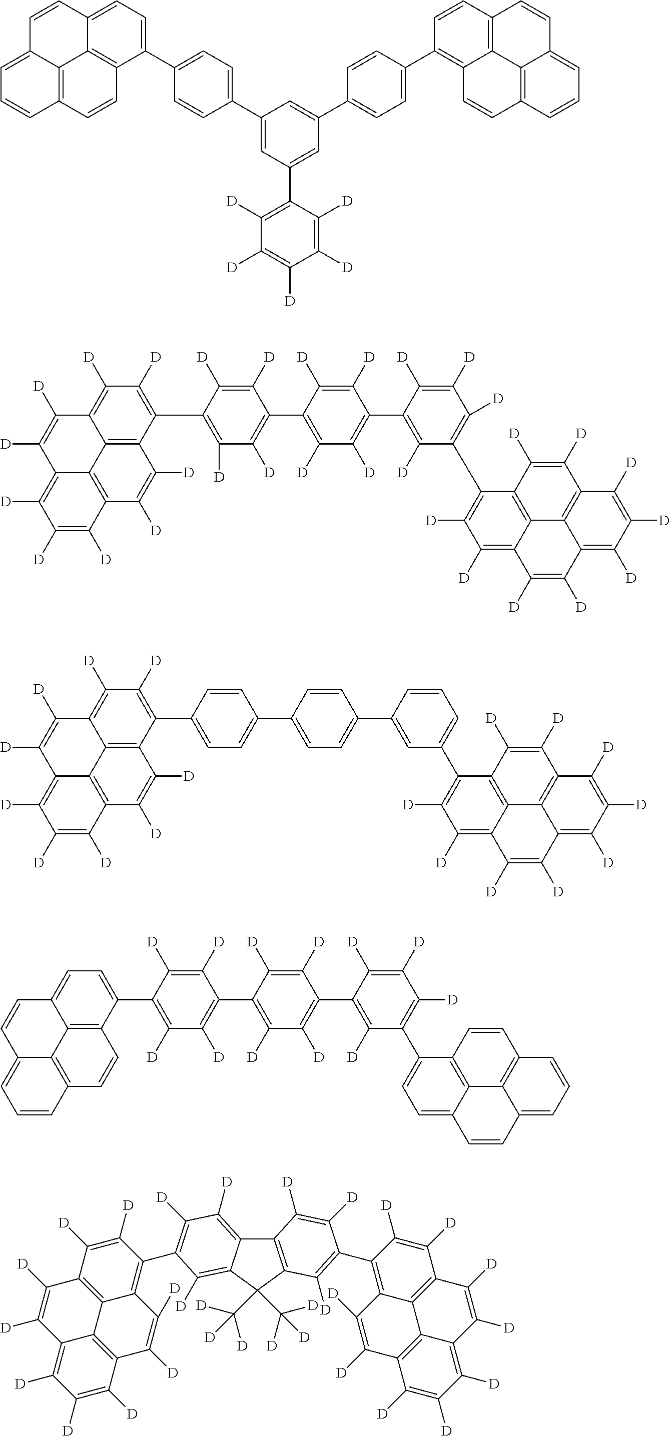

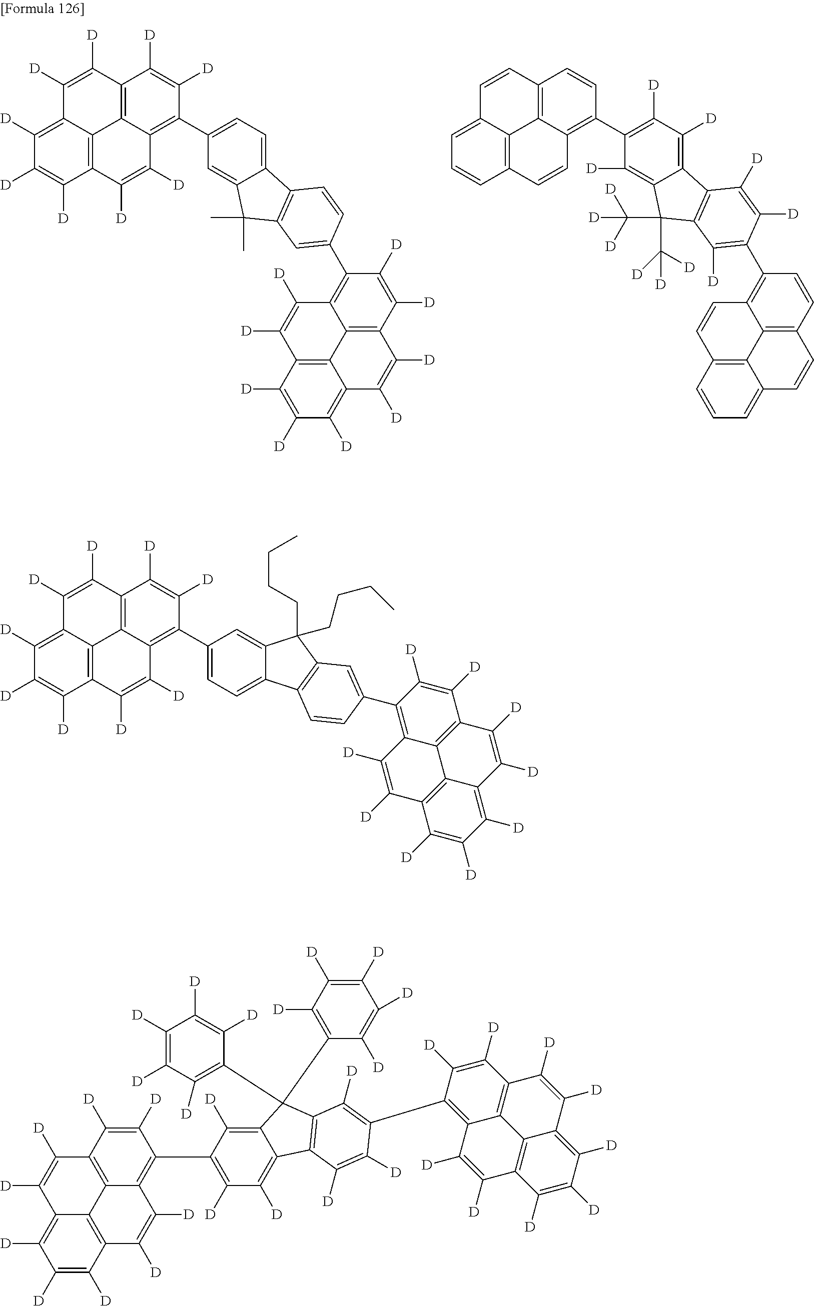

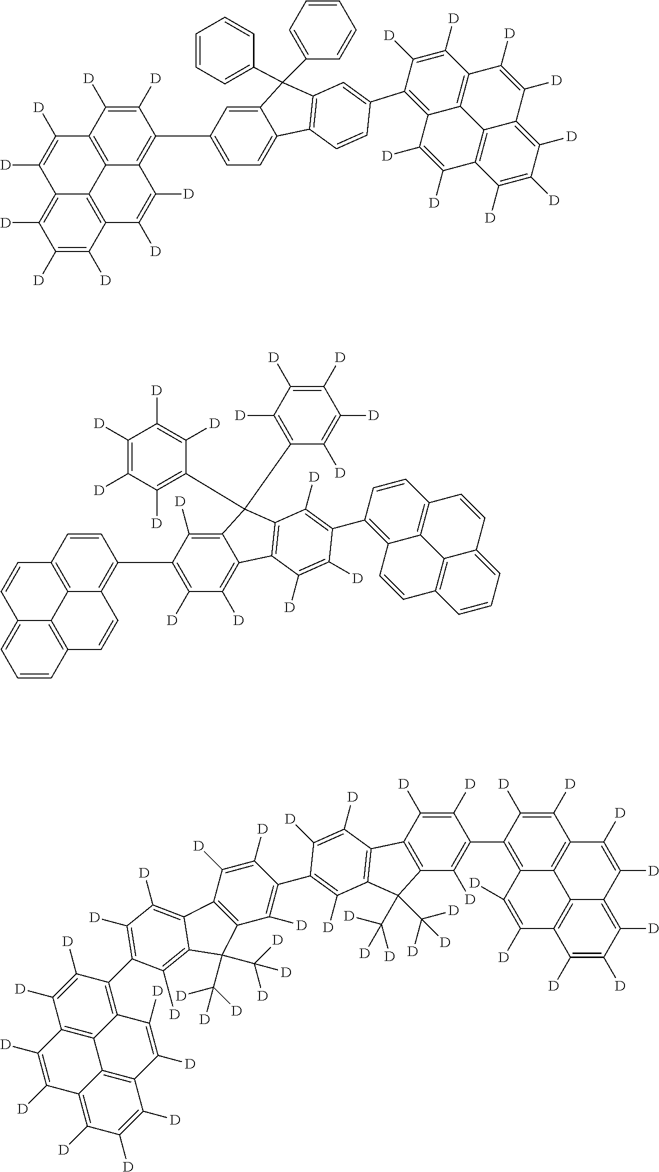

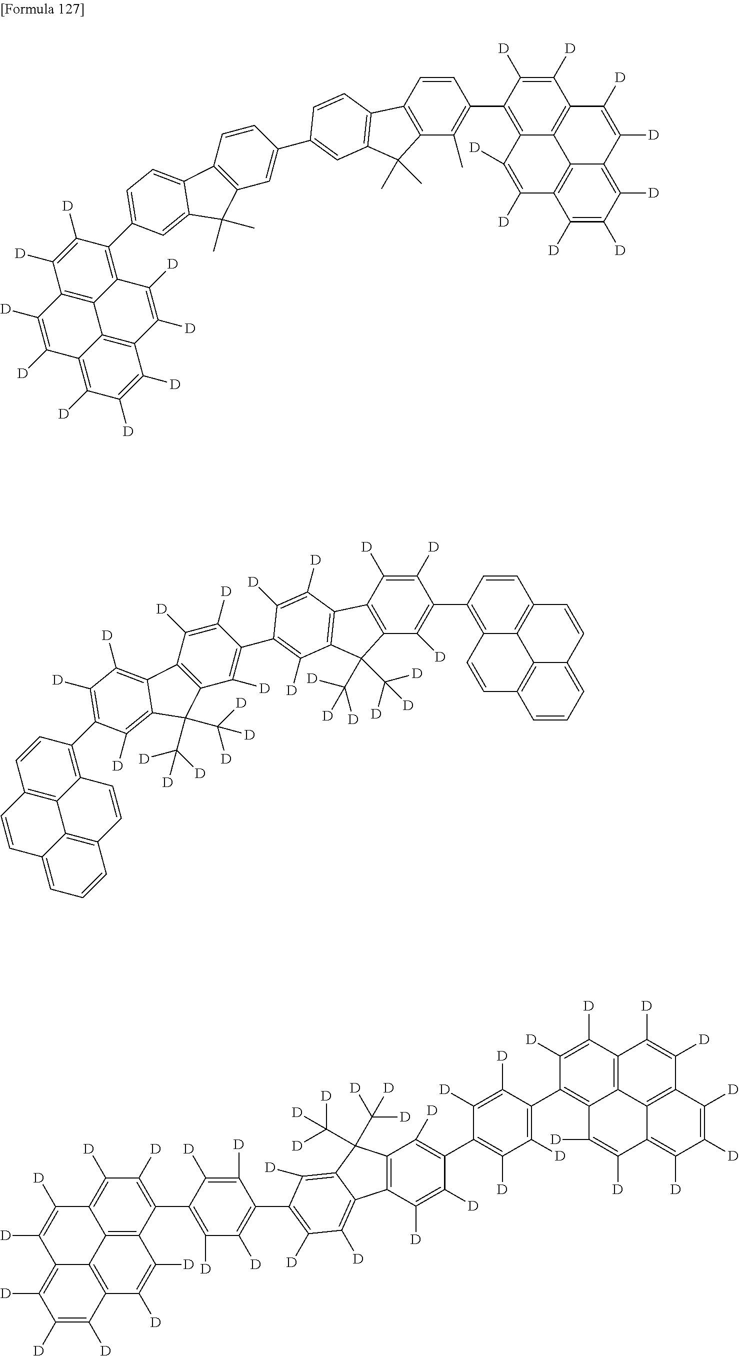

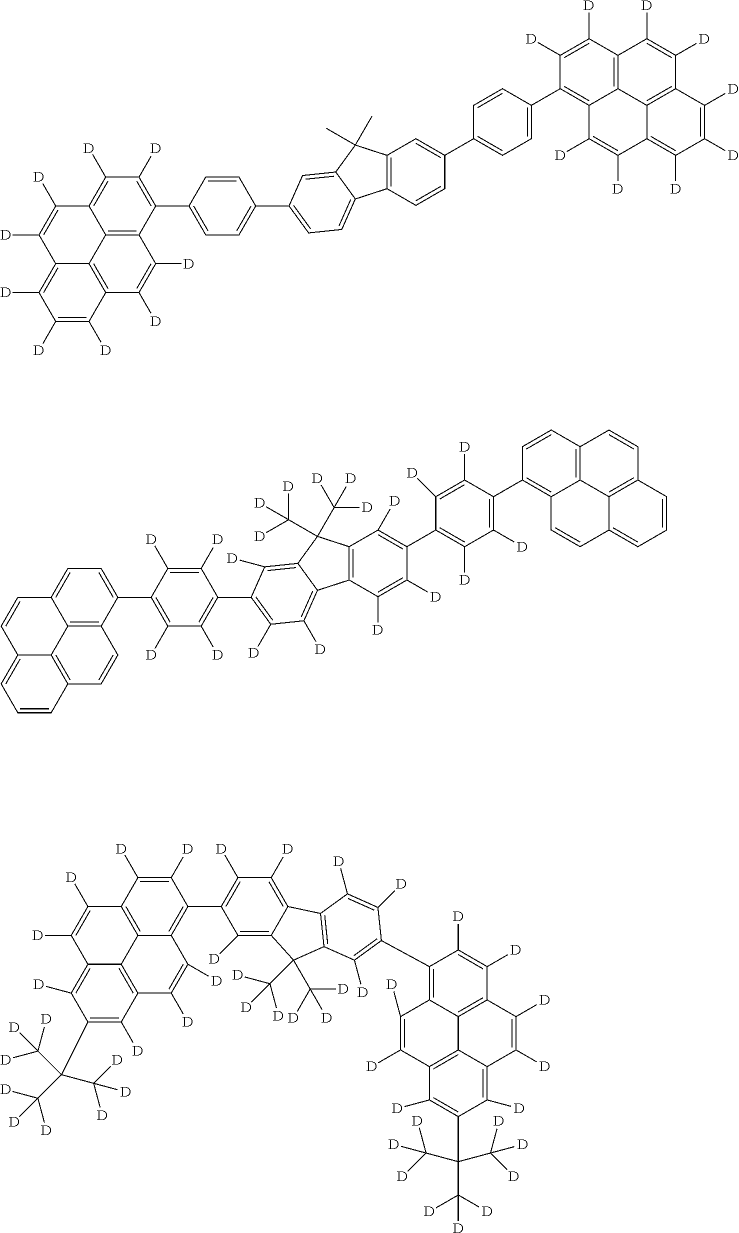

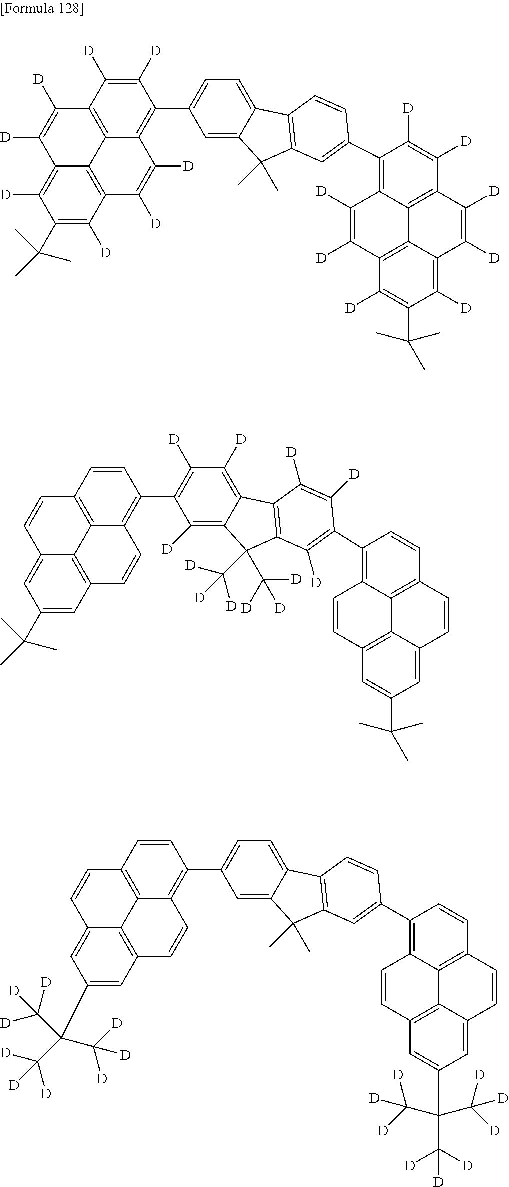

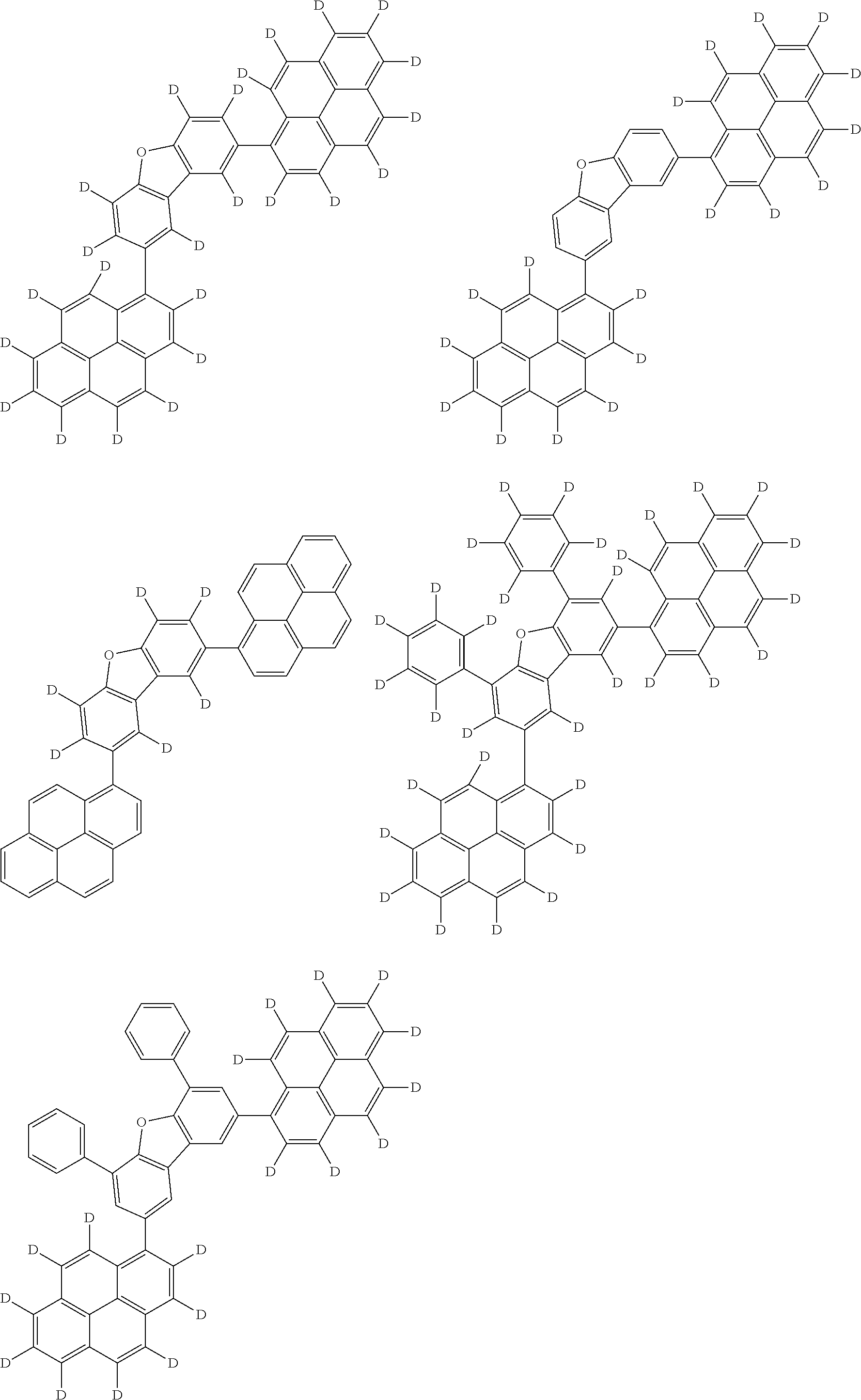

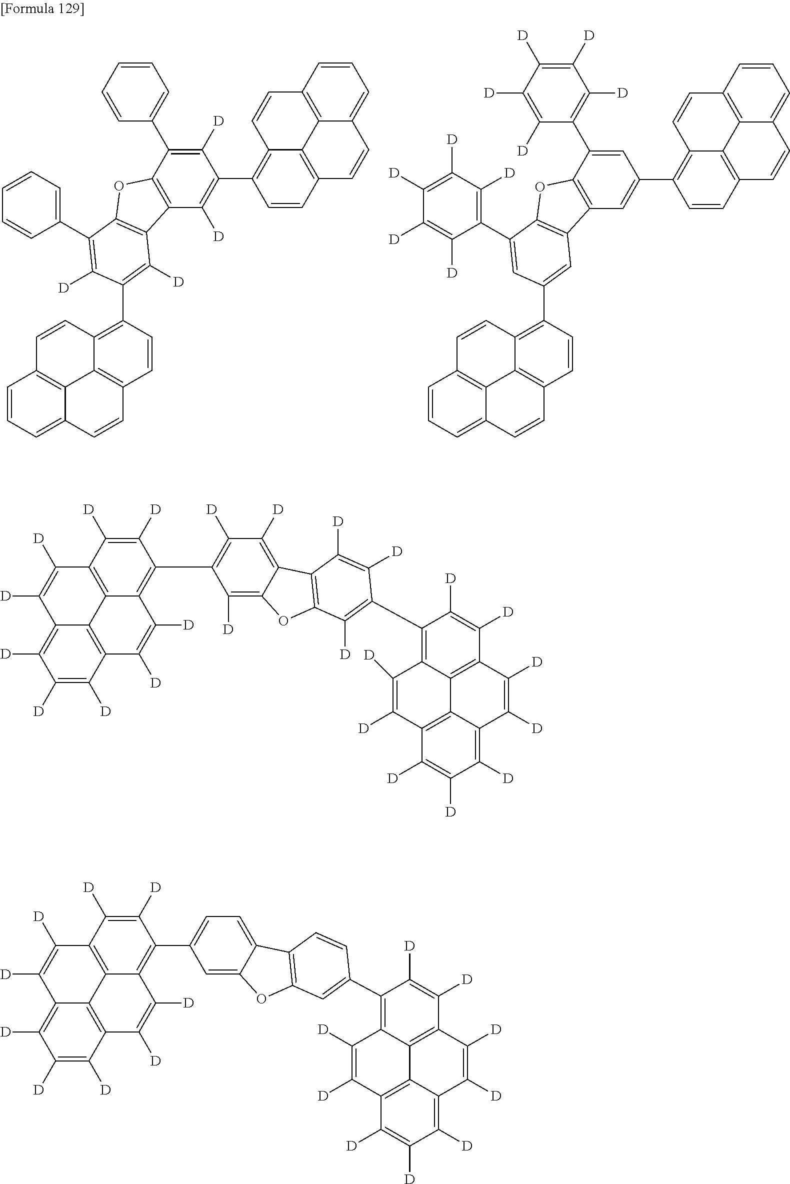

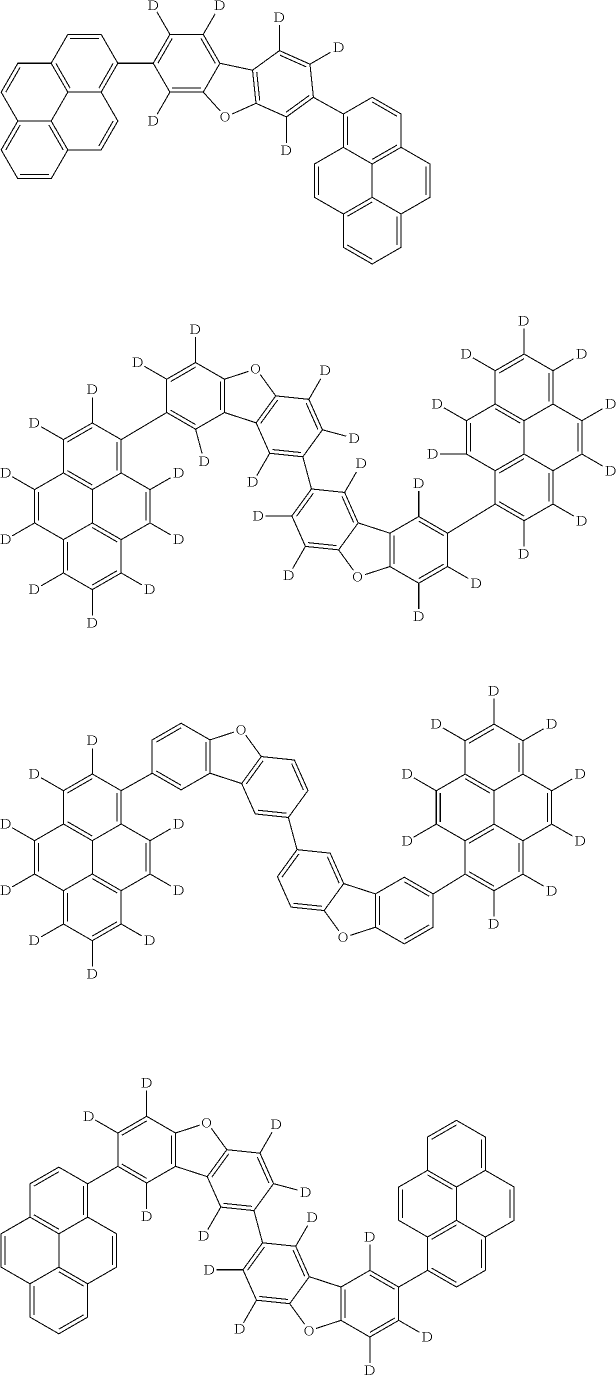

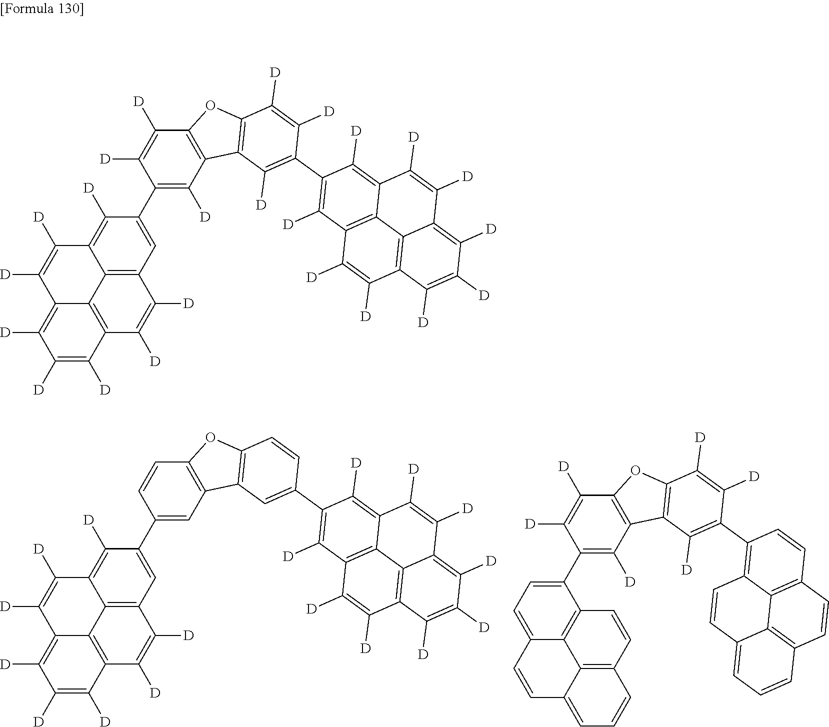

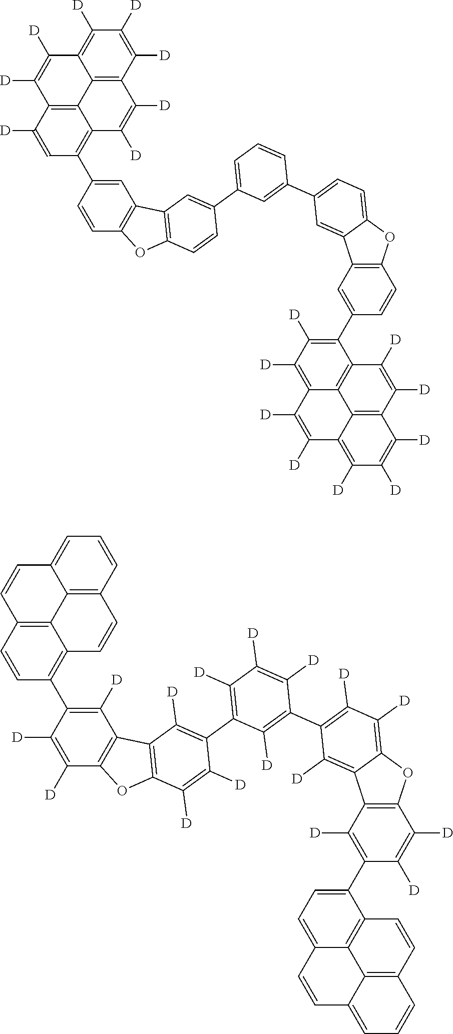 [SMILES: [2H]c1c([2H])c2c([2H])c([2H])c3c([2H])cc(-c4ccc5oc6ccc(-c7cccc(-c8ccc9oc%10ccc(-c%11c([2H])c([2H])c%12c([2H])c([2H])c%13c([2H])c([2H])c([2H])c%14c([2H])c([2H])c%11c%12c%13%14)cc%10c9c8)c7)cc6c5c4)c4c([2H])c([2H])c(c1[2H])c2c34 ; [2H]c1c([2H])c(-c2c([2H])c([2H])c3oc4c([2H])c([2H])c(-c5ccc6ccc7cccc8ccc5c6c78)c([2H])c4c3c2[2H])c([2H])c(-c2c([2H])c([2H])c3oc4c([2H])c([2H])c(-c5ccc6ccc7cccc8ccc5c6c78)c([2H])c4c3c2[2H])c1[2H]]

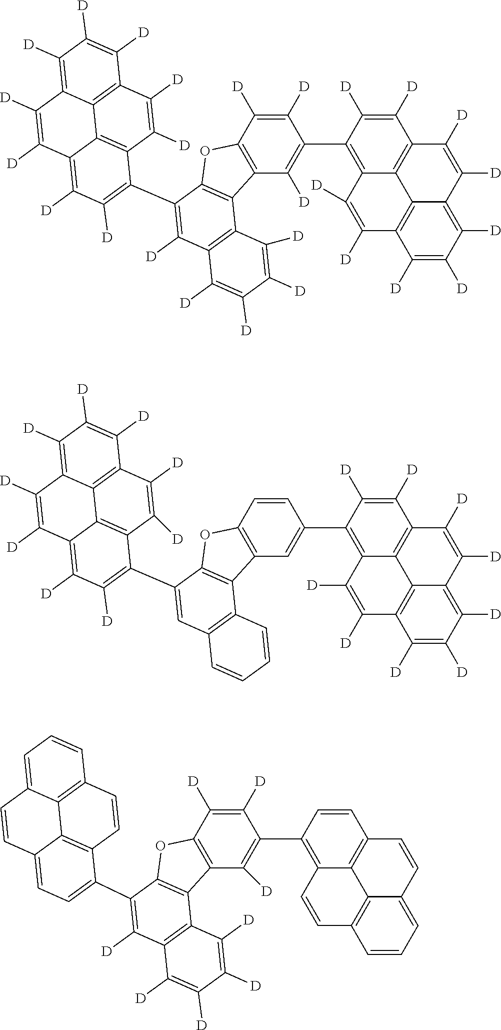

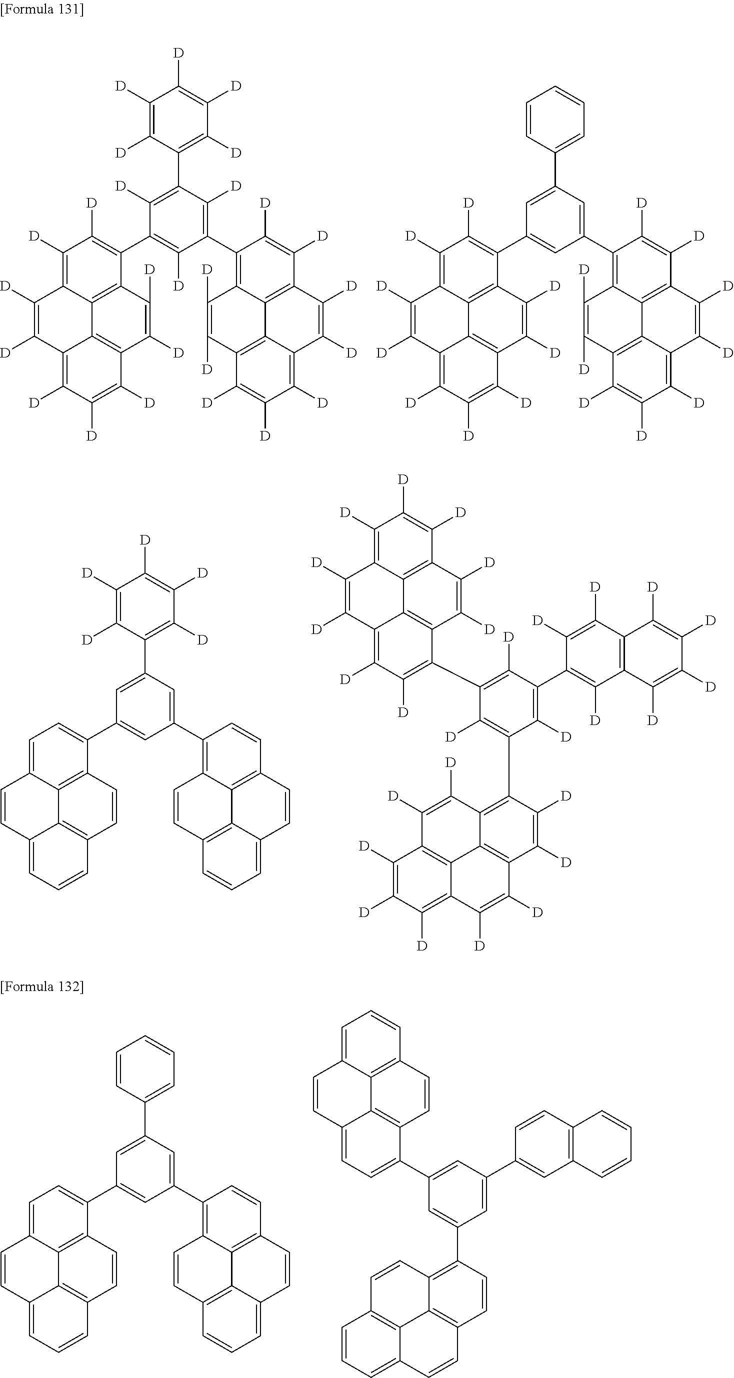

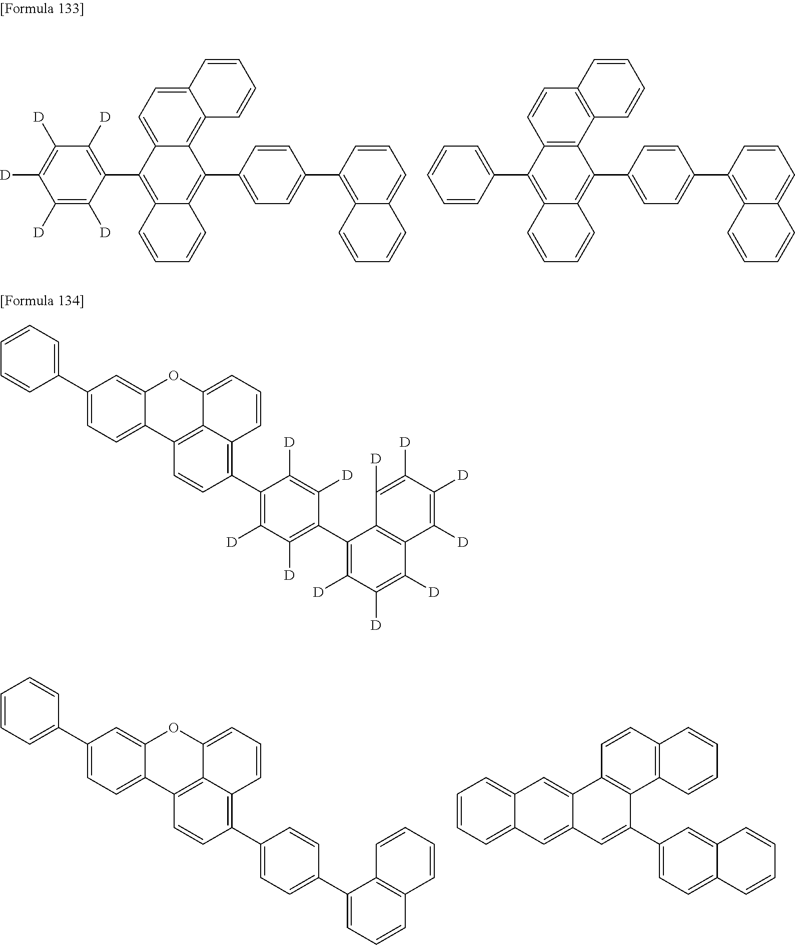

### Second Compound

In the organic EL device according to the exemplary embodiment, the second compound is a compound represented by the formula (2) below.

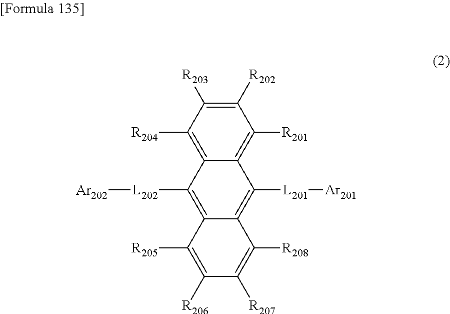

In the formula (2):
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₂₀₁ to L₂₀₂ are each independently a single bond, a substituted or unsubstituted arylene group having 6 to 50 ring carbon atoms, or a substituted or unsubstituted divalent heterocyclic group having 5 to 50 ring atoms; and
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms.

In the second compound according to the exemplary embodiment: R₉₀₁, R₉₀₂, R₉₀₃, R₉₀₄, R₉₀₅, R₉₀₆, R₉₀₇, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In the organic EL device according to the exemplary embodiment, it is preferable that R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group or a nitro group;
L₂₀₁ to L₂₀₂ are each independently a single bond, a substituted or unsubstituted arylene group having 6 to 50 ring carbon atoms, or a substituted or unsubstituted divalent heterocyclic group having 5 to 50 ring atoms; and
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms.

In the organic EL device according to the exemplary embodiment, it is preferable that L₂₀₁ to L₂₀₂ are each independently a single bond or a substituted or unsubstituted arylene group having 6 to 50 ring carbon atoms; and
Ar₂₀₁ and Ar₂₀₂ are each independently a substituted or unsubstituted aryl group having 6 to 50 ring carbon atoms.

In the organic EL device according to the exemplary embodiment, it is preferable that Ar₂₀₁ and Ar₂₀₂ are each independently a phenyl group, a naphthyl group, phenanthryl group, a biphenyl group, a terphenyl group, a diphenylfluorenyl group, a dimethylfluorenyl group, benzodiphenylfluorenyl group, a benzodimethylfluorenyl group, a dibenzofuranyl group, dibenzothienyl group, a naphthobenzofuranyl group, or a naphthobenzothienyl group.

In the organic EL device according to the exemplary embodiment, the second compound represented by the formula (2) is preferably a compound represented by a formula (201), (202), (203), (204), (205), (206), (207), (208) or (209) below.

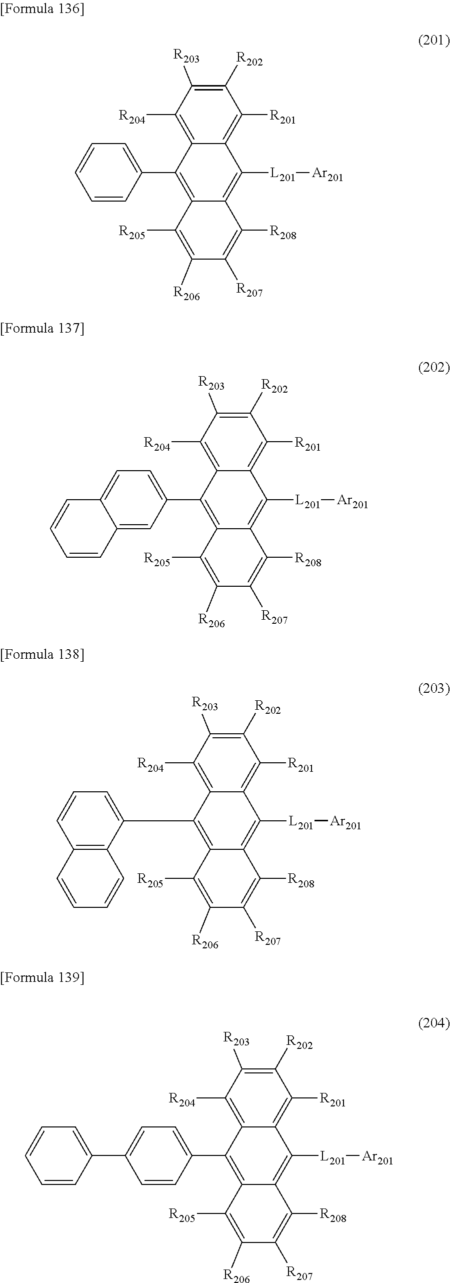

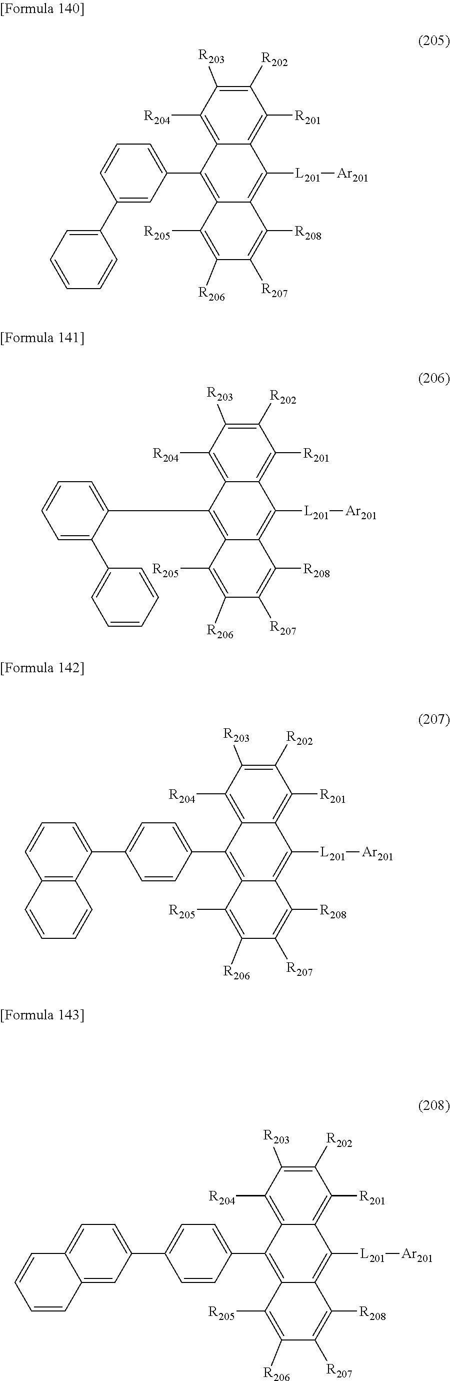

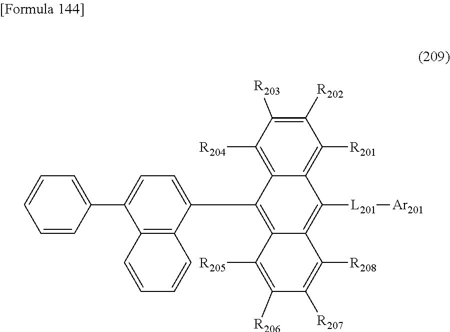

In the formulae (201) to (209):
L₂₀₁ and Ar₂₀₁ represent the same as L₂₀₁ and Ar₂₀₁ in the formula (2); and
R₂₀₁ to R₂₀₈ each independently represent the same as R₂₀₁ to R₂₀₈ in the formula (2).

It is also preferable that the second compound represented by the formula (2) is a compound represented by a formula (221), a formula (222), a formula (223), a formula (224), a formula (225), a formula (226), a formula (227), a formula (228), or a formula (229) below.

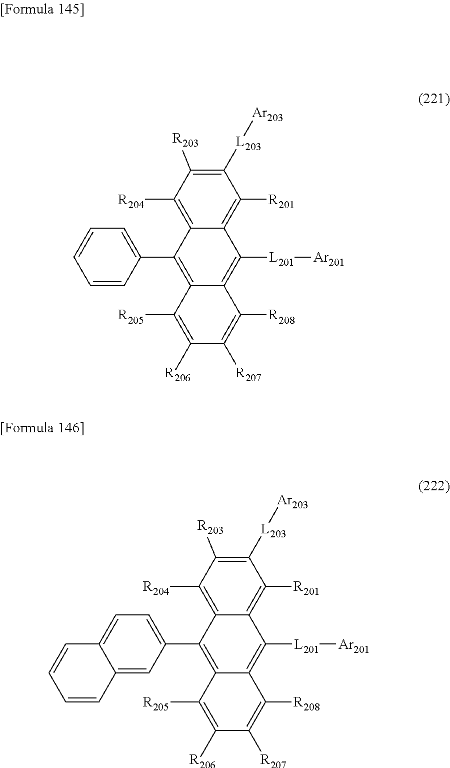

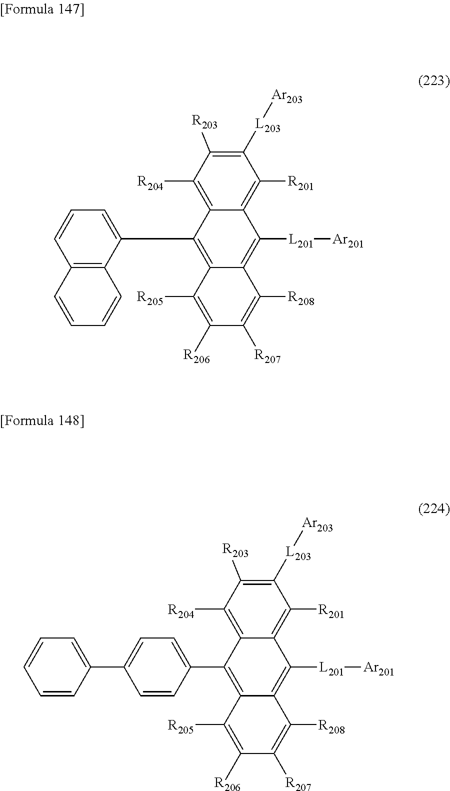

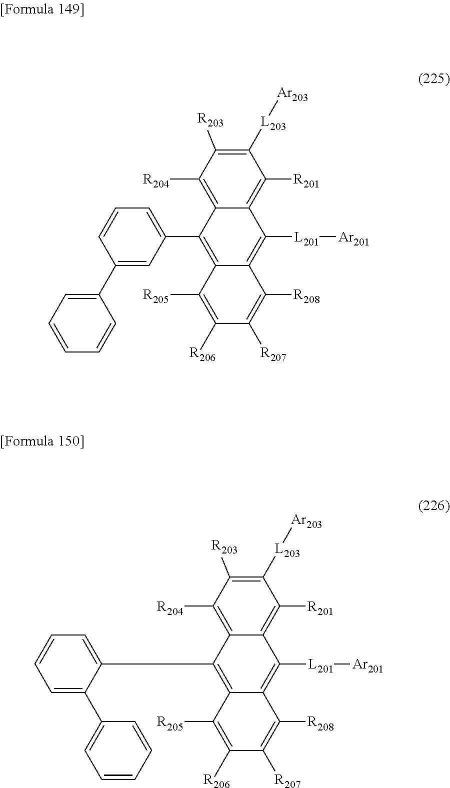

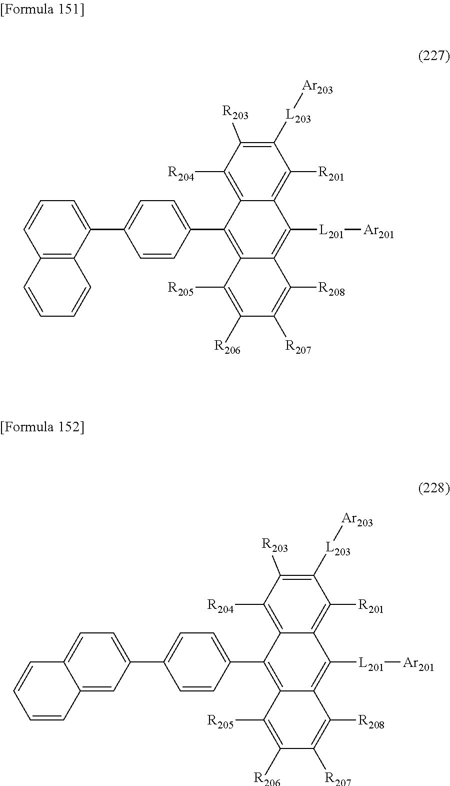

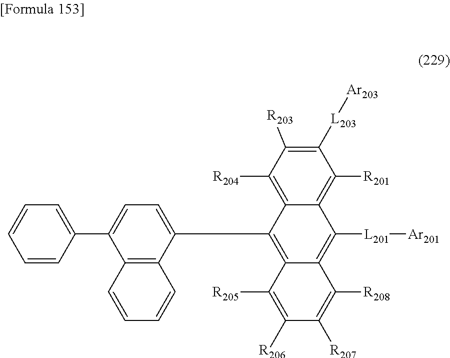

In the formulae (221), (222), (223), (224), (225), (226), (227), (228) and (229):
R₂₀₁ and R₂₀₃ to R₂₀₈ each independently represent the same as R₂₀₁ and R₂₀₃ to R₂₀₈ in the formula (2);
L₂₀₁ and Ar₂₀₁ represent the same as L₂₀₁ and Ar₂₀₁ in the formula (2); and
L₂₀₃ represents the same as L₂₀₁ in the formula (2);
L₂₀₃ and L₂₀₁ are mutually the same or different;
Ar₂₀₃ represents the same as Ar₂₀₁ in the formula (2); and
Ar₂₀₃ and Ar₂₀₁ are mutually the same or different.

The second compound represented by the formula (2) is also preferably a compound represented by a formula (241), (242), (243), (244), (245), (246), (247), (248) or (249).

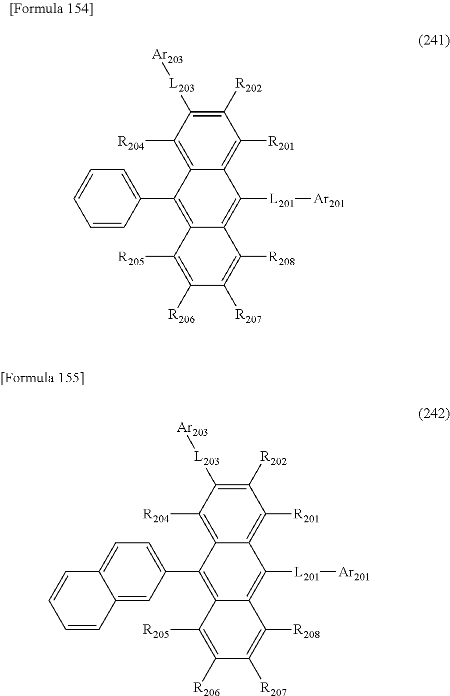

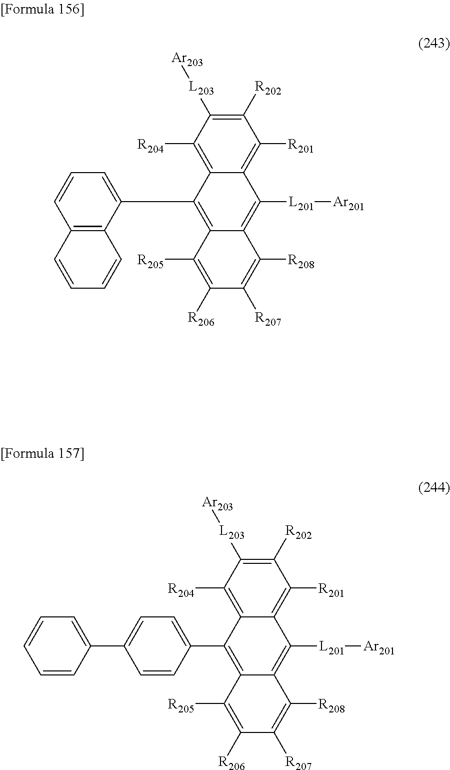

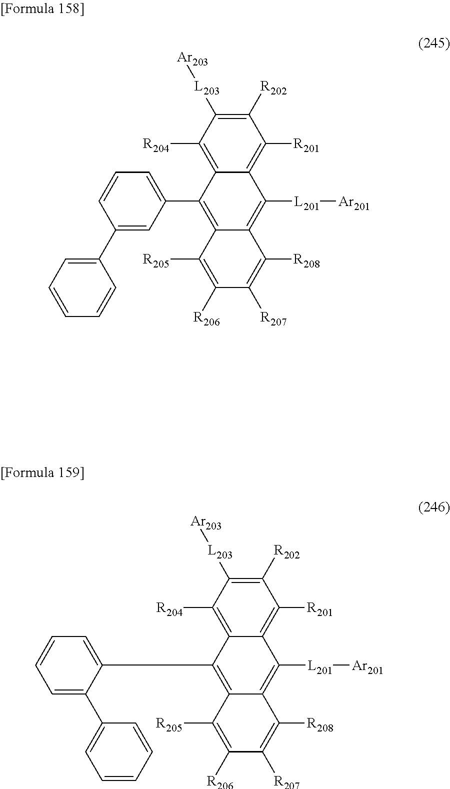

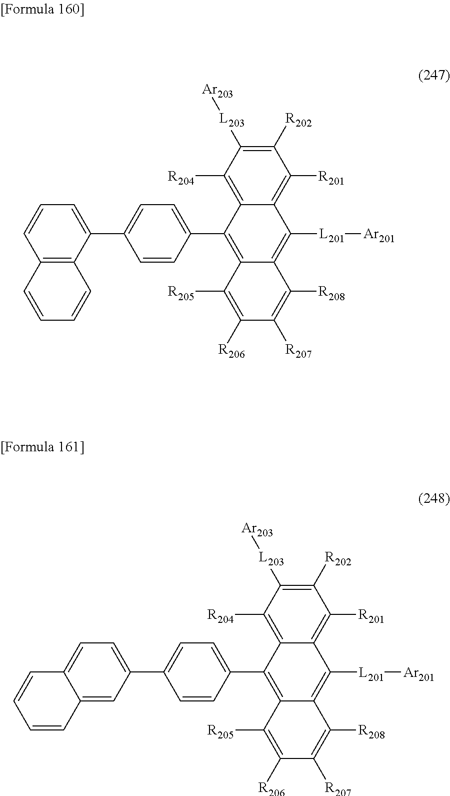

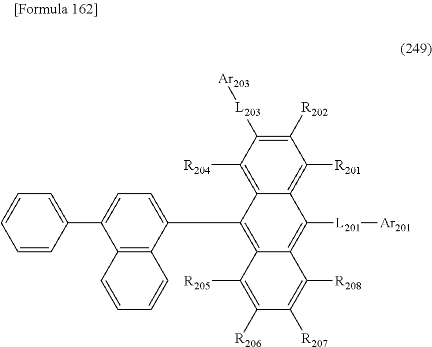

In the formulae (241), (242), (243), (244), (245), (246), (247), (248) and (249):
R₂₀₁, R₂₀₂, and R₂₀₄ to R₂₀₈ each independently represent the same as R₂₀₁, R₂₀₂, and R₂₀₄ to R₂₀₈ in the formula (2);
L₂₀₃ represents the same as L₂₀₁ in the formula (2);
L₂₀₃ and L₂₀₁ are mutually the same or different;
Ar₂₀₃ represents the same as Ar₂₀₁ in the formula (2); and
Ar₂₀₃ and Ar₂₀₁ are mutually the same or different.

In the second compound represented by the formula (2), R₂₀₁ to R₂₀₈ that are not represented by the formula (21) are each independently preferably a hydrogen atom, a substituted or unsubstituted alkyl group having 1 to 50 carbon atoms, a substituted or unsubstituted cycloalkyl group having 3 to 50 ring carbon atoms, or a group represented by —Si(R₉₀₁)(R₉₀₂)(R₉₀₃).

L₁₀₁ is preferably a single bond or an unsubstituted arylene group having 6 to 22 ring carbon atoms, and
Ar₁₀₁ is preferably a substituted or unsubstituted aryl group having 6 to 22 ring carbon atoms.

In the organic EL device according to the exemplary embodiment, R₂₀₁ to R₂₀₈ that are substituents on an anthracene skeleton in the second compound represented by the formula (2) are preferably hydrogen atoms in terms of preventing inhibition of intermolecular interaction to inhibit a decrease in electron mobility. However, R₂₀₁ to R₂₀₈ may be a substituted or unsubstituted aryl group having 6 to 50 ring carbon atoms or a substituted or unsubstituted heterocyclic group having 5 to 50 ring atoms.

Assuming that R₂₀₁ to R₂₀₈ each are a bulky substituent such as an alkyl group and a cycloalkyl group, intermolecular interaction may be inhibited to decrease the electron mobility of the second compound relative to that of the first host material, so that a relationship of μH2>μH1 shown by the numerical formula (Numerical Formula 6) may not be satisfied. When the second compound is used in the second emitting layer, it can be expected that satisfying the relationship of μH2>μH1 inhibits a decrease in a recombination ability between holes and electrons in the first emitting layer and a decrease in a luminous efficiency. It should be noted that as the substituent, namely, a haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O)R₈₀₁, group represented by —COOR₈₀₂, halogen atom, cyano group, and nitro group are likely to be bulky, and an alkyl group and cycloalkyl group are likely to be further bulky.

In the second compound represented by the formula (2), R₂₀₁ to R₂₀₈, which are the substituents on the anthracene skeleton, are each preferably not a bulky substituent and preferably not an alkyl group and cycloalkyl group. More preferably, R₂₀₁ to R₂₀₈ are not an alkyl group, cycloalkyl group, haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O)R₈₀₁, group represented by —COOR₈₀₂, halogen atom, cyano group, and nitro group.

In the organic EL device according to the exemplary embodiment, it is also preferable that in the second compound represented by the formula (2), R₂₀₁ to R₂₀₈ are each independently a hydrogen atom, a substituted or unsubstituted alkyl group having 1 to 50 carbon atoms, a substituted or unsubstituted cycloalkyl group having 3 to 50 ring carbon atoms, or a group represented by —Si(R₉₀₁)(R₉₀₂)(R₉₀₃).

In the organic EL device according to the exemplary embodiment, R₂₀₁ to R₂₀₈ in the second compound represented by the formula (2) are each preferably a hydrogen atom.

In the second compound, examples of a substituent for a “substituted or unsubstituted” group on R₂₀₁ to R₂₀₈ also preferably do not include the above-described substituent that is likely to be bulky, especially a substituted or unsubstituted alkyl group and a substituted or unsubstituted cycloalkyl group. Since the examples of the substituent for the “substituted or unsubstituted” group on R₂₀₁ to R₂₀₈ do not include a substituted or unsubstituted alkyl group and a substituted or unsubstituted cycloalkyl group, inhibition of intermolecular interaction to be caused by presence of a bulky substituent such as an alkyl group and a cycloalkyl group can be prevented, thereby preventing a decrease in the electron mobility. Moreover, when the second compound described above is used in the second emitting layer, a decrease in a recombination ability between holes and electrons in the first emitting layer and a decrease in the luminous efficiency can be inhibited.

It is more preferably that R₂₀₁ to R₂₀₈, which are the substituents on the anthracene skeleton, are not bulky substituents, and R₂₀₁ to R₂₀₈ as substituents are unsubstituted. Assuming that R₂₀₁ to R₂₀₈, which are the substituents on the anthracene skeleton, are not bulky substituents and R₂₀₁ to R₂₀₈ that are not bulky substituents are bonded with a substituent, the substituent is preferably not a bulky substituent; the substituent bonded to R₂₀₁ to R₂₀₈ serving as substituents is preferably not an alkyl group and cycloalkyl group, more preferably not an alkyl group, cycloalkyl group, haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O)R₈₀₁, group represented by —COOR₈₀₂, halogen atom, cyano group, and nitro group.

In the second compound, the groups specified to be “substituted or unsubstituted” are each preferably an “unsubstituted” group.

### Manufacturing Method of Second Compound

The second compound can be manufactured by a known method. The second compound can also be manufactured based on a known method through a known alternative reaction using a known material(s) tailored for the target compound.

### Specific Examples of Second Compound

Specific examples of the second compound include the following compounds. It should however be noted that the invention is not limited by the specific examples of the second compound.

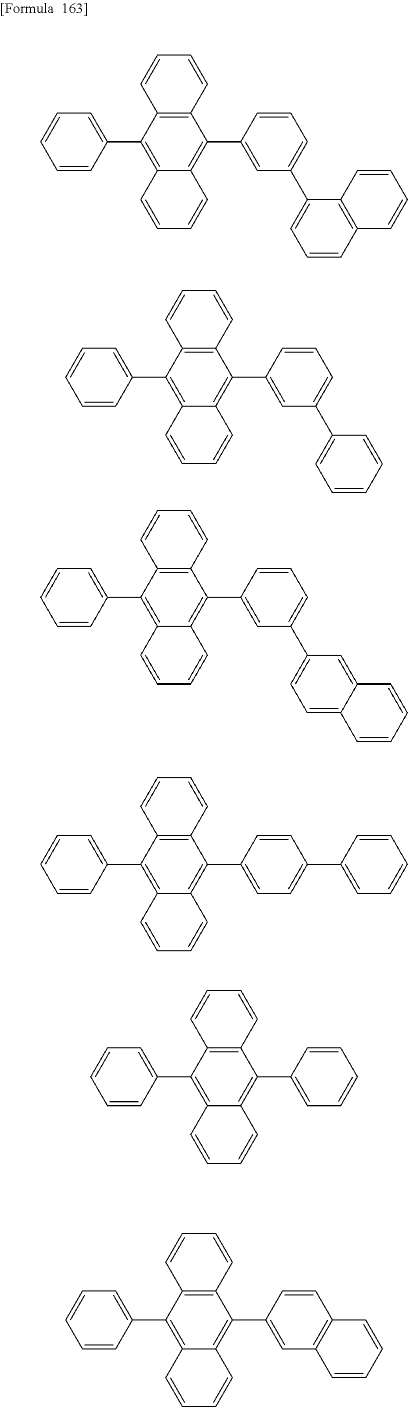

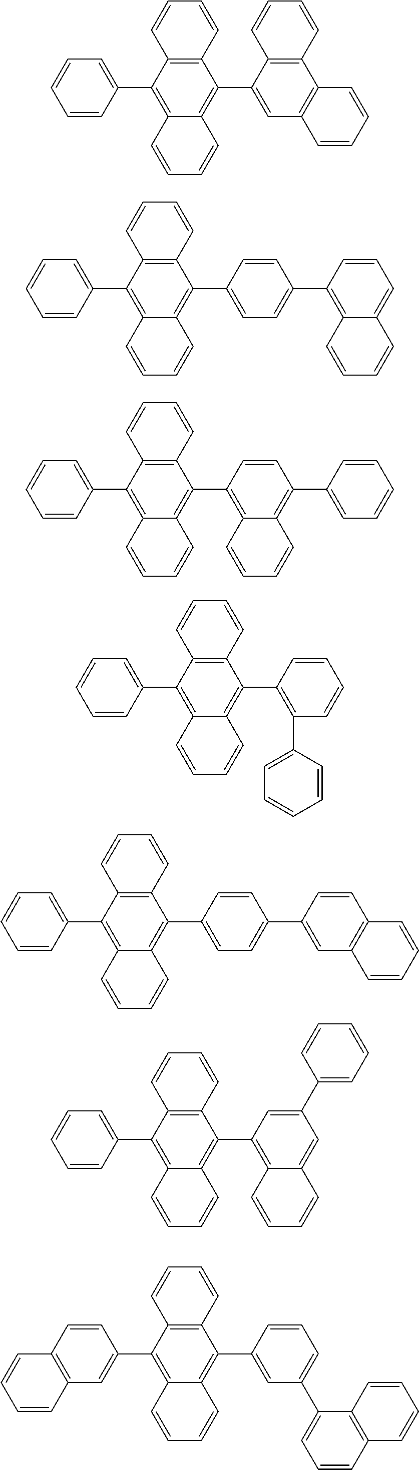

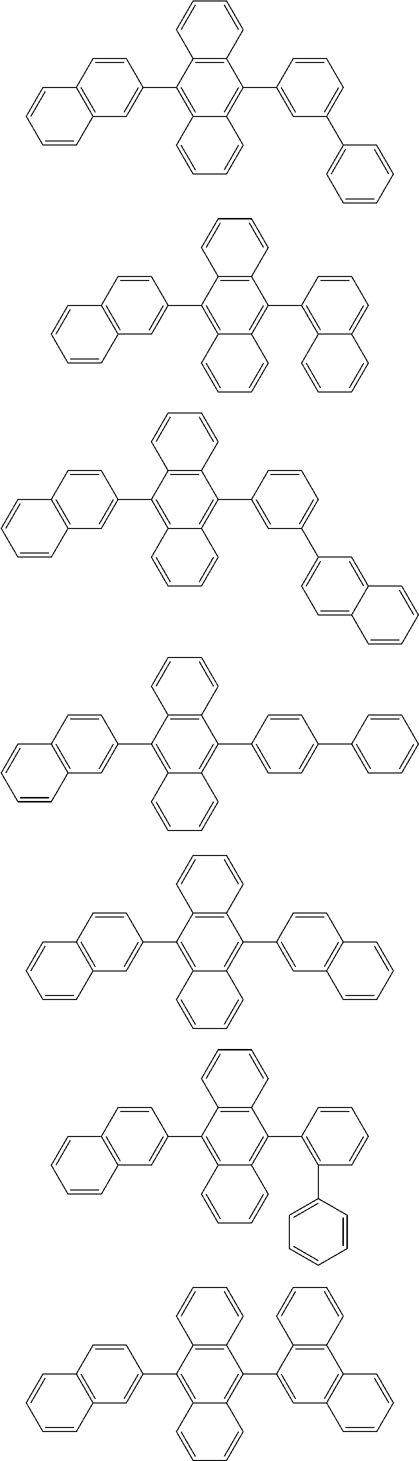

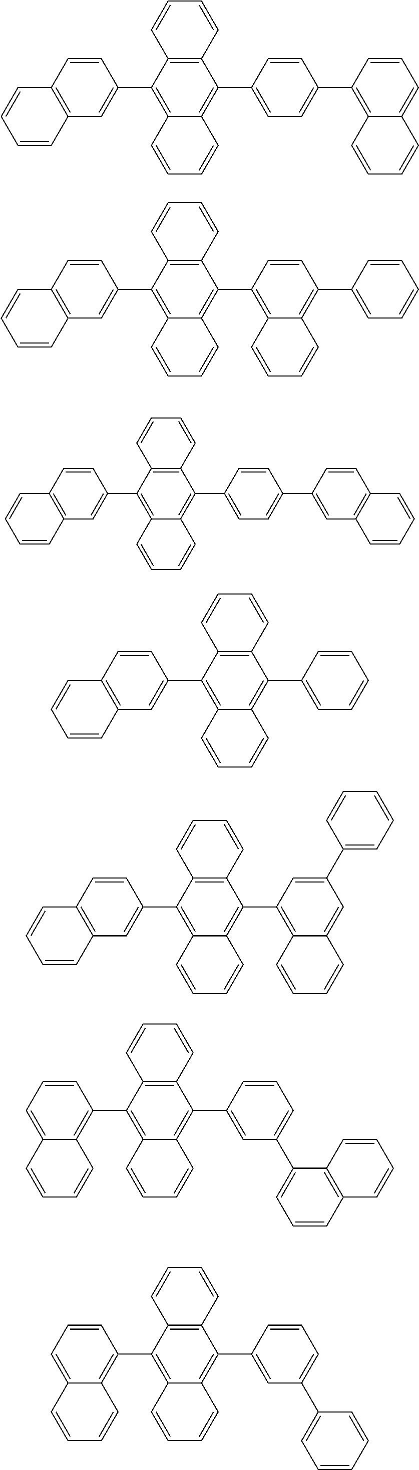

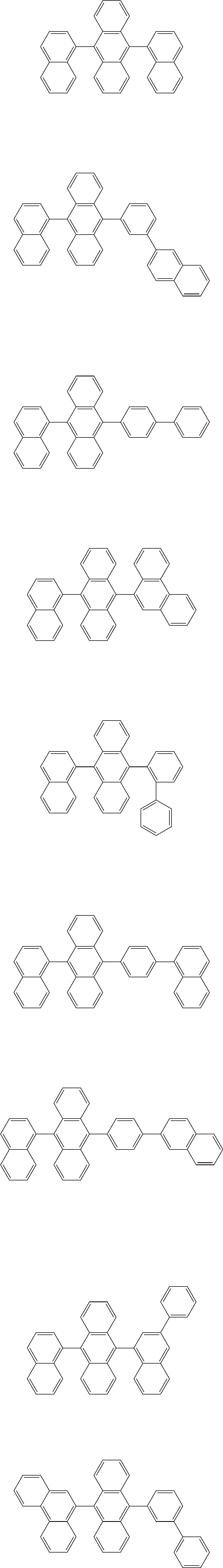

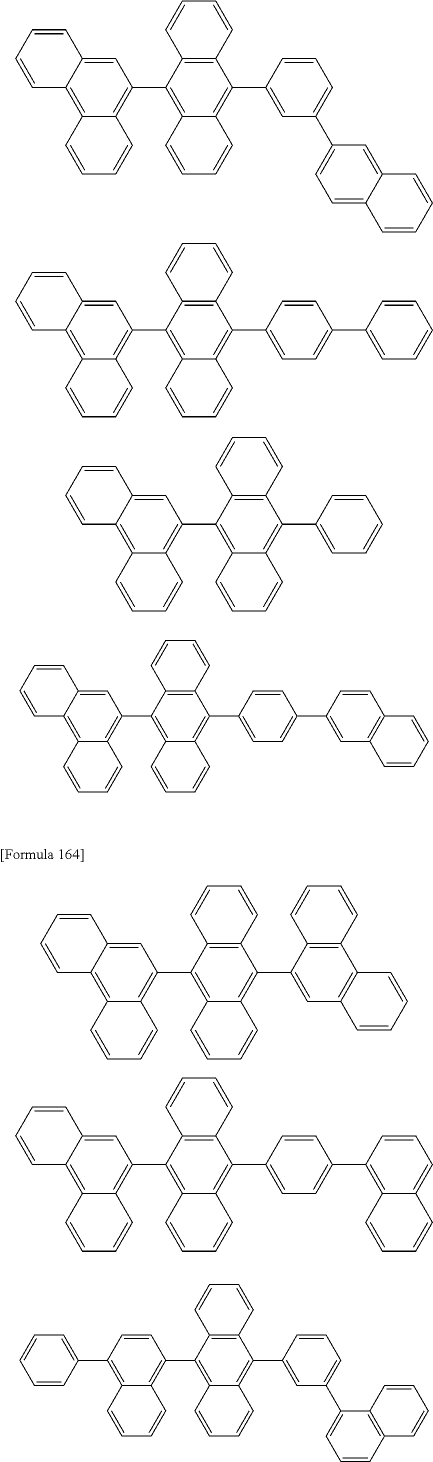

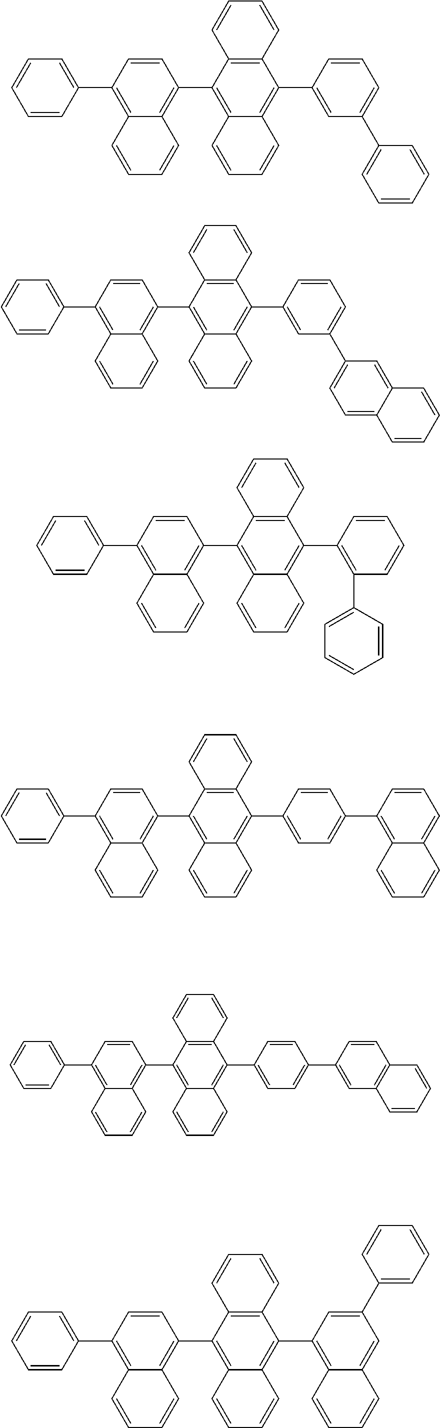

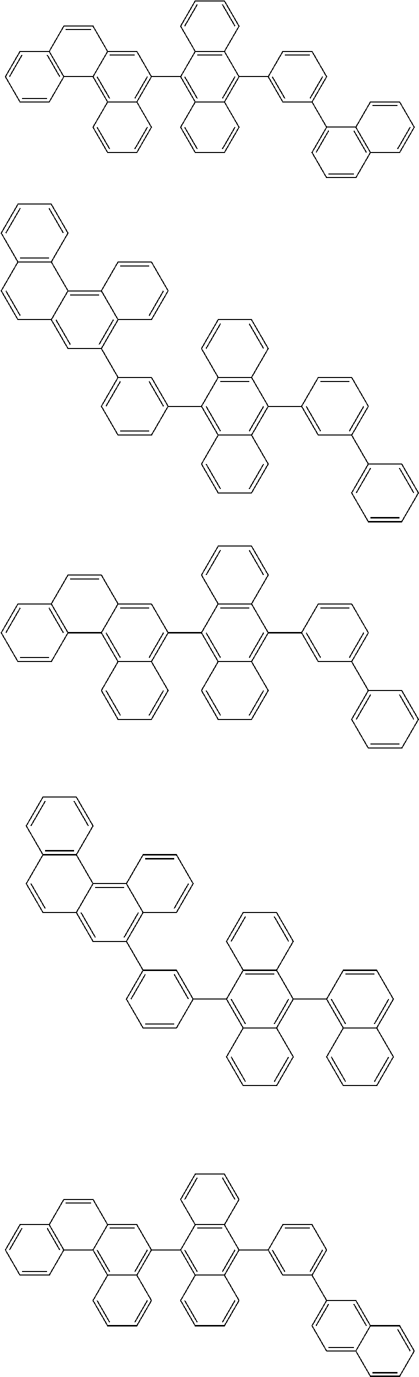

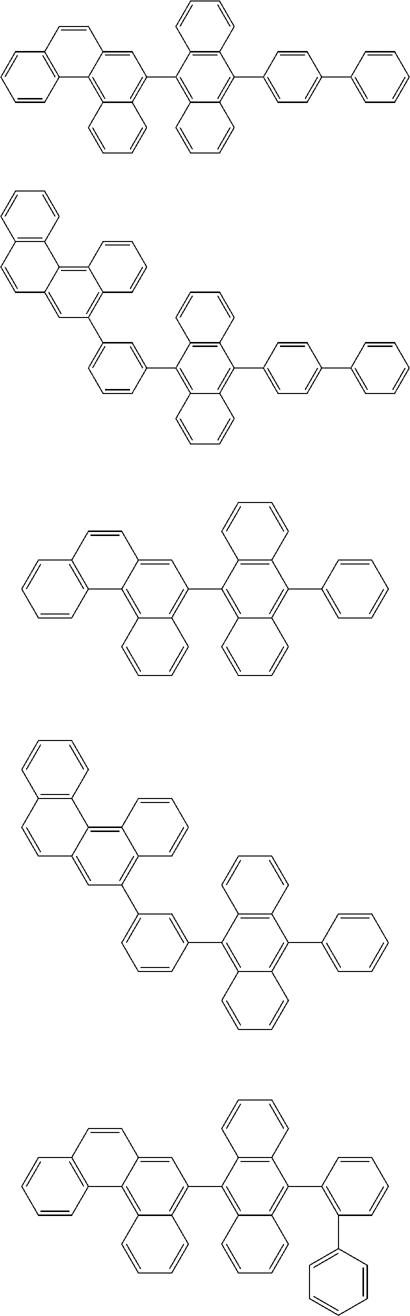

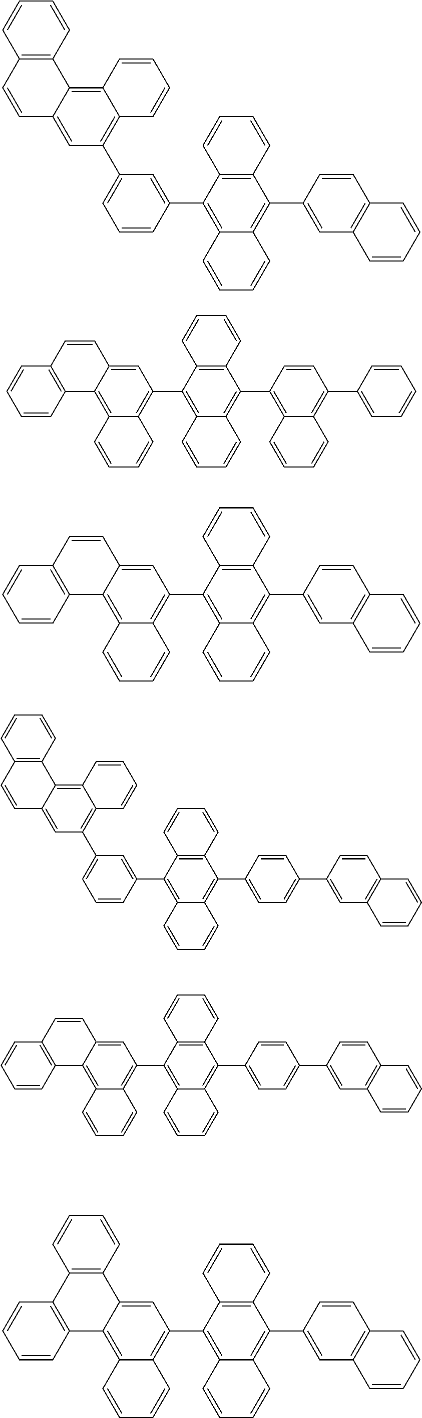

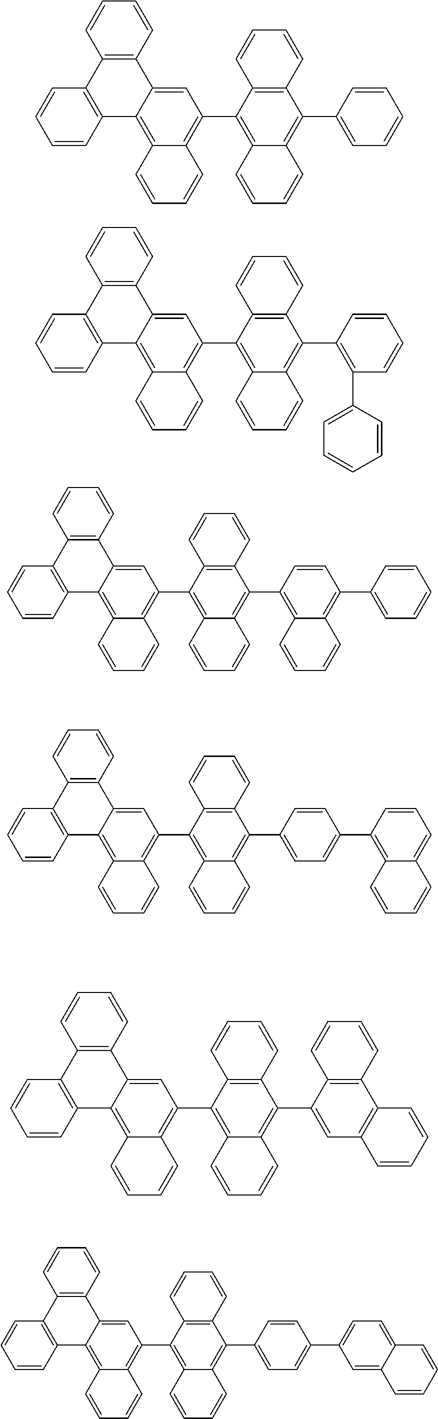

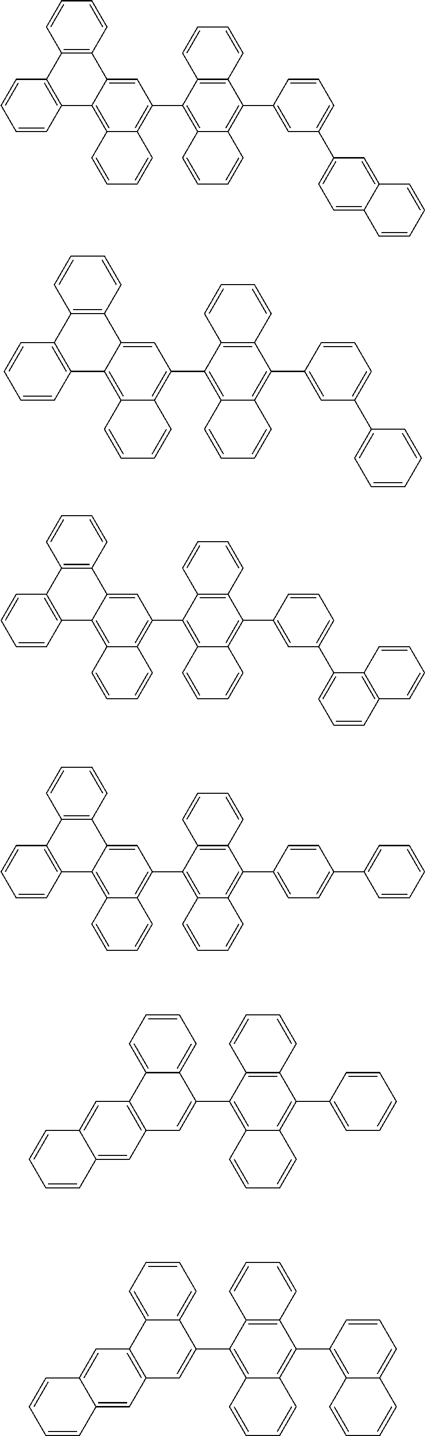

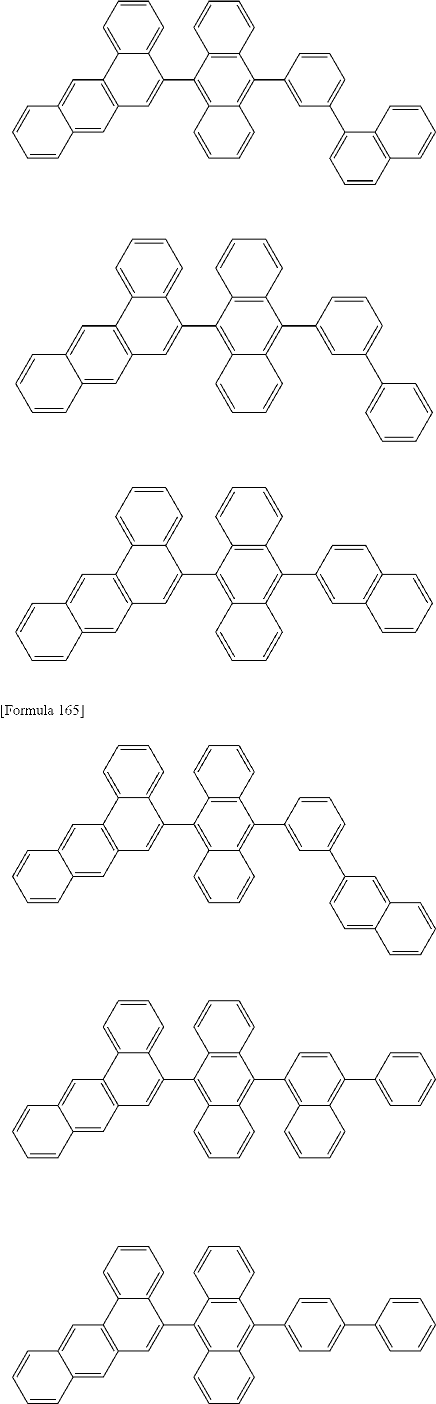

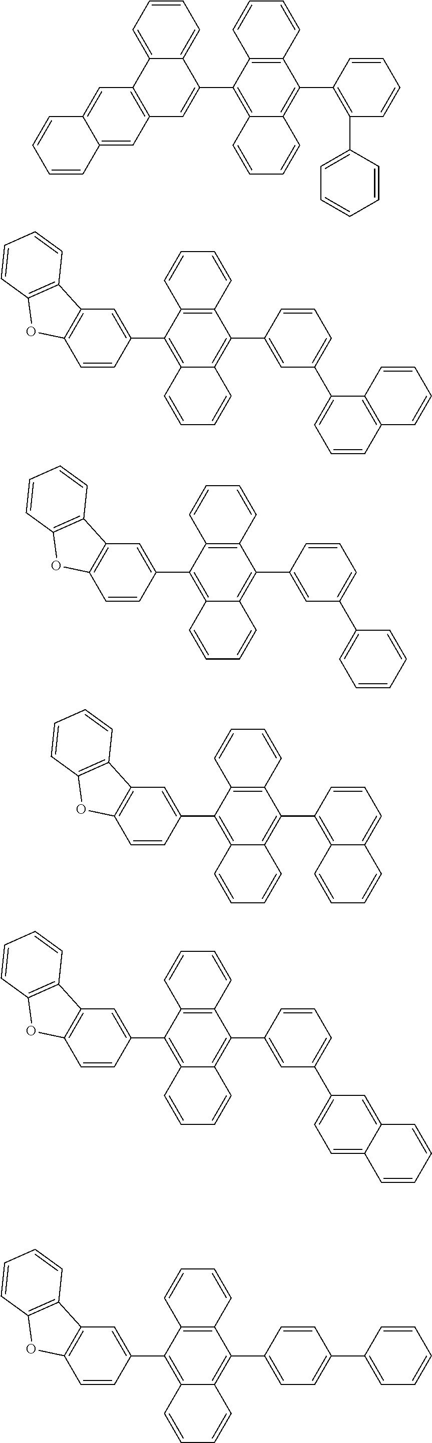

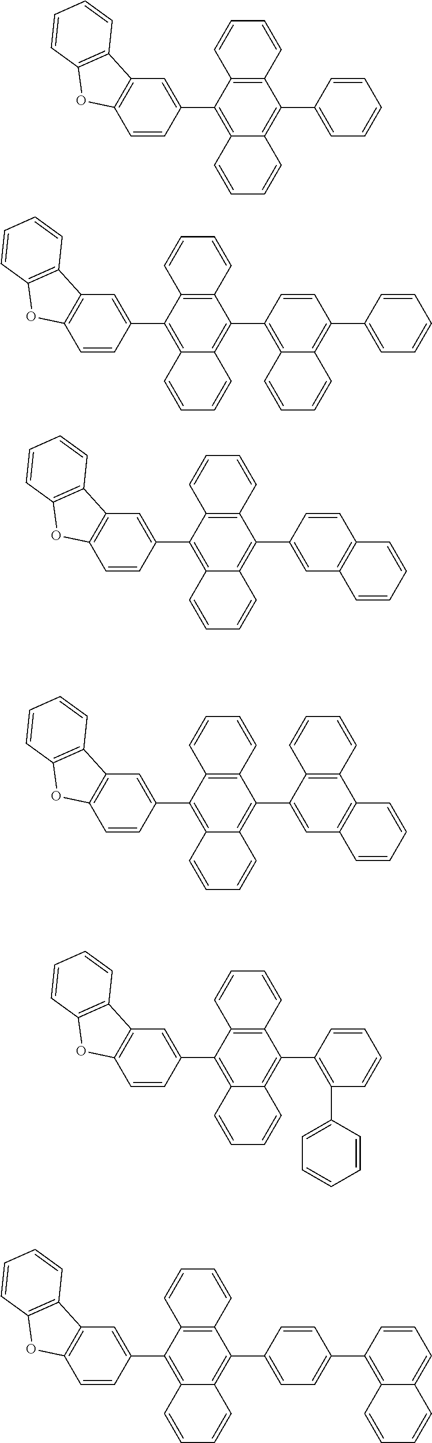

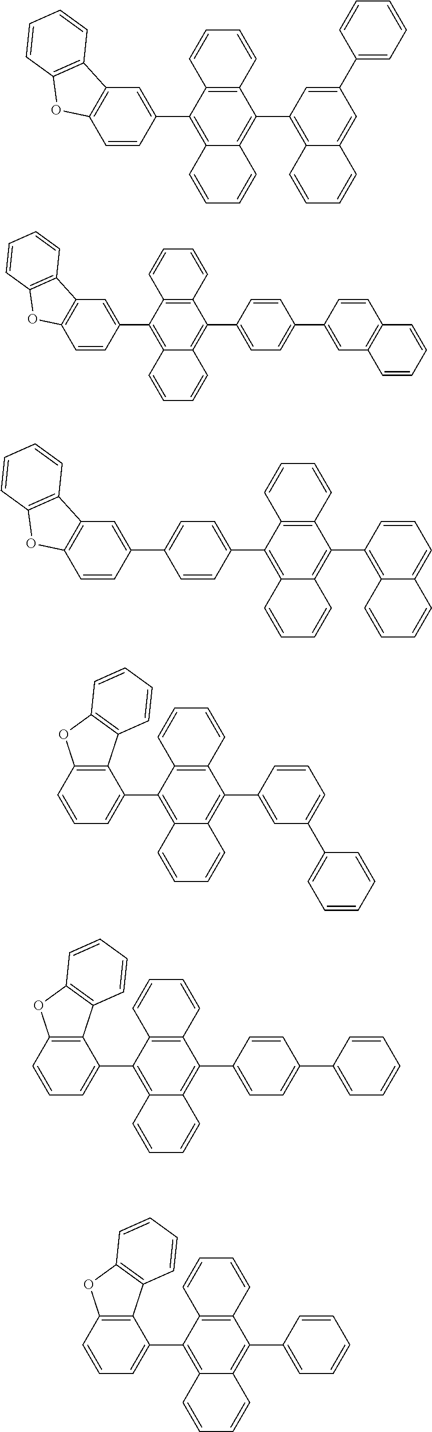 [SMILES: c1ccc(-c2cc(-c3c4ccccc4c(-c4ccc5oc6ccccc6c5c4)c4ccccc34)c3ccccc3c2)cc1 ; c1ccc2cc(-c3ccc(-c4c5ccccc5c(-c5ccc6oc7ccccc7c6c5)c5ccccc45)cc3)ccc2c1 ; c1ccc2c(-c3c4ccccc4c(-c4ccc(-c5ccc6oc7ccccc7c6c5)cc4)c4ccccc34)cccc2c1 ; c1ccc(-c2cccc(-c3c4ccccc4c(-c4cccc5oc6ccccc6c45)c4ccccc34)c2)cc1 ; c1ccc(-c2ccc(-c3c4ccccc4c(-c4cccc5oc6ccccc6c45)c4ccccc34)cc2)cc1 ; c1ccc(-c2c3ccccc3c(-c3cccc4oc5ccccc5c34)c3ccccc23)cc1]

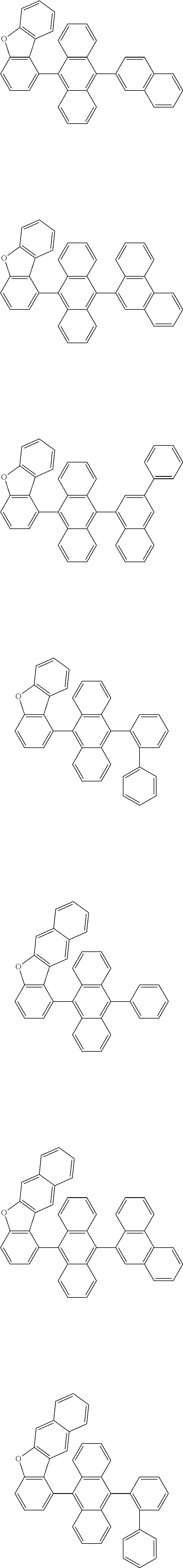

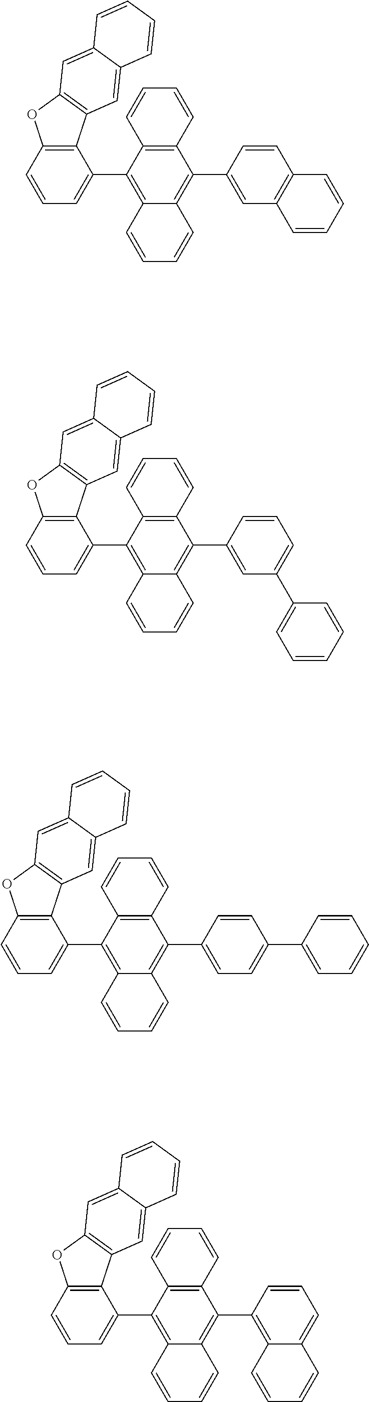 [SMILES: c1ccc2cc(-c3c4ccccc4c(-c4cccc5oc6cc7ccccc7cc6c45)c4ccccc34)ccc2c1 ; c1ccc(-c2cccc(-c3c4ccccc4c(-c4cccc5oc6cc7ccccc7cc6c45)c4ccccc34)c2)cc1 ; c1ccc(-c2ccc(-c3c4ccccc4c(-c4cccc5oc6cc7ccccc7cc6c45)c4ccccc34)cc2)cc1 ; c1ccc2cc3c(cc2c1)oc1cccc(-c2c4ccccc4c(-c4cccc5ccccc45)c4ccccc24)c13]

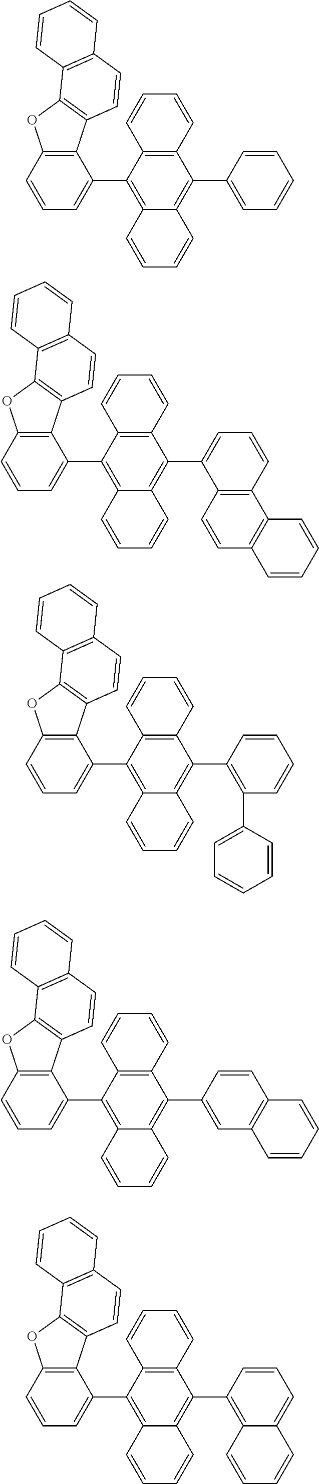 [SMILES: c1ccc(-c2c3ccccc3c(-c3cccc4oc5c6ccccc6ccc5c34)c3ccccc23)cc1 ; c1ccc2c(c1)ccc1c(-c3c4ccccc4c(-c4cccc5oc6c7ccccc7ccc6c45)c4ccccc34)cccc12 ; c1ccc(-c2ccccc2-c2c3ccccc3c(-c3cccc4oc5c6ccccc6ccc5c34)c3ccccc23)cc1 ; c1ccc2cc(-c3c4ccccc4c(-c4cccc5oc6c7ccccc7ccc6c45)c4ccccc34)ccc2c1 ; c1ccc2c(-c3c4ccccc4c(-c4cccc5oc6c7ccccc7ccc6c45)c4ccccc34)cccc2c1]

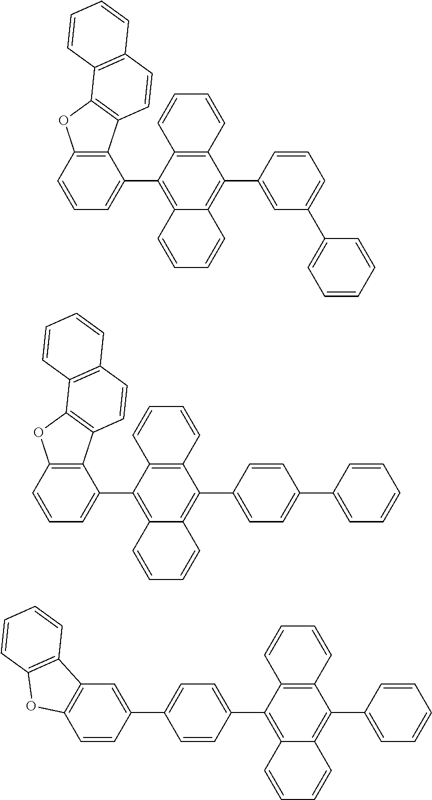 [SMILES: c1ccc(-c2cccc(-c3c4ccccc4c(-c4cccc5oc6c7ccccc7ccc6c45)c4ccccc34)c2)cc1 ; c1ccc(-c2ccc(-c3c4ccccc4c(-c4cccc5oc6c7ccccc7ccc6c45)c4ccccc34)cc2)cc1 ; c1ccc(-c2c3ccccc3c(-c3ccc(-c4ccc5oc6ccccc6c5c4)cc3)c3ccccc23)cc1]

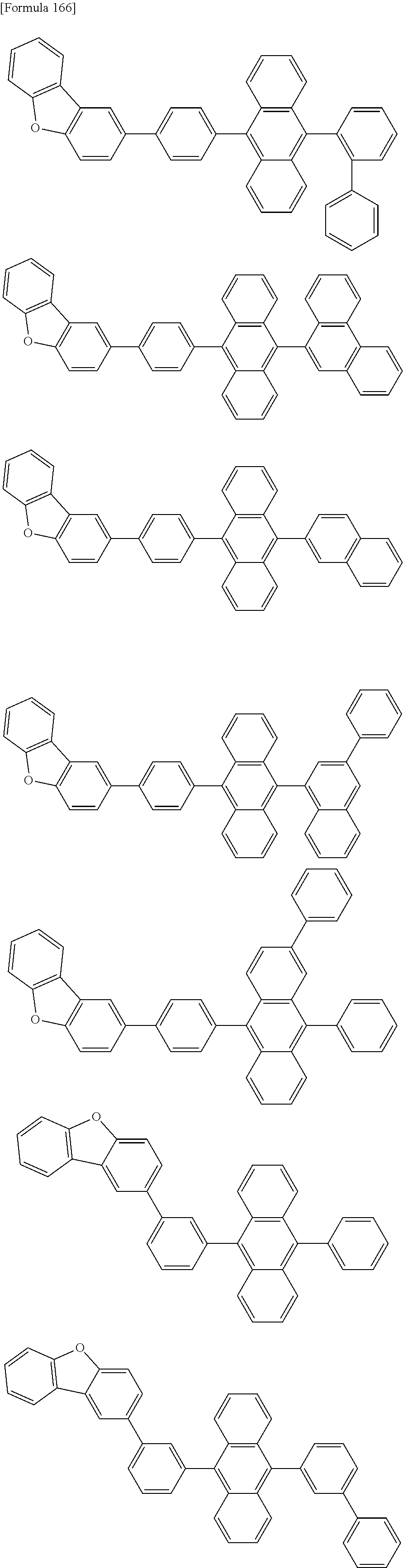

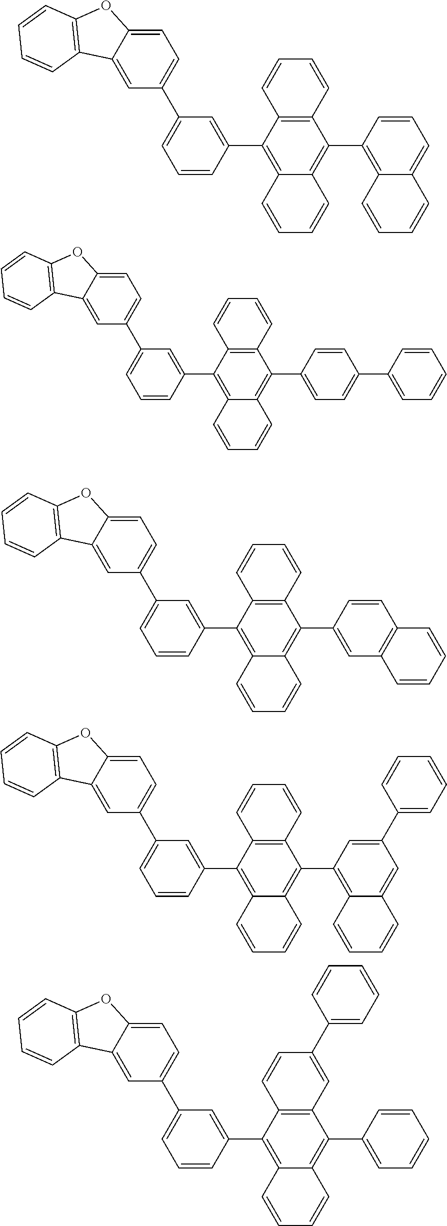

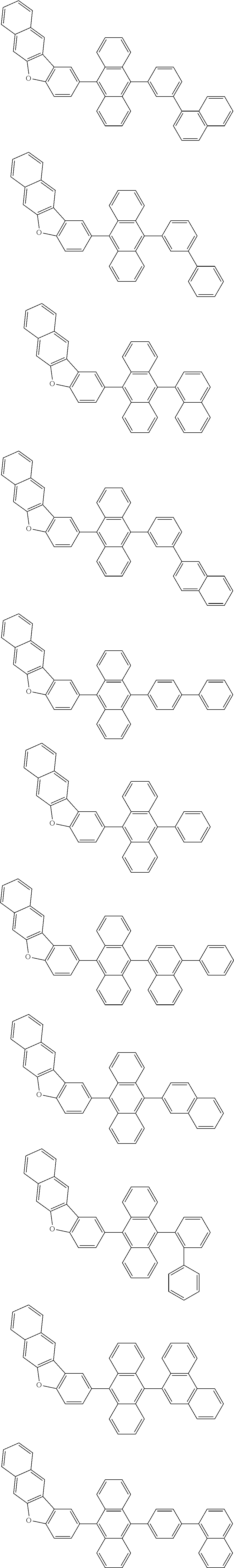

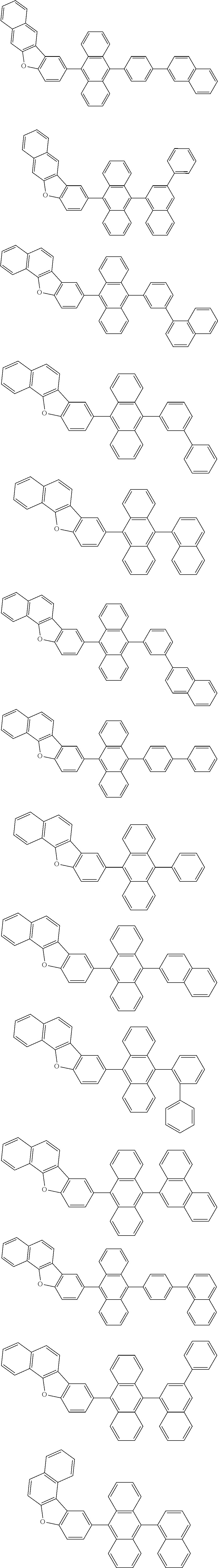

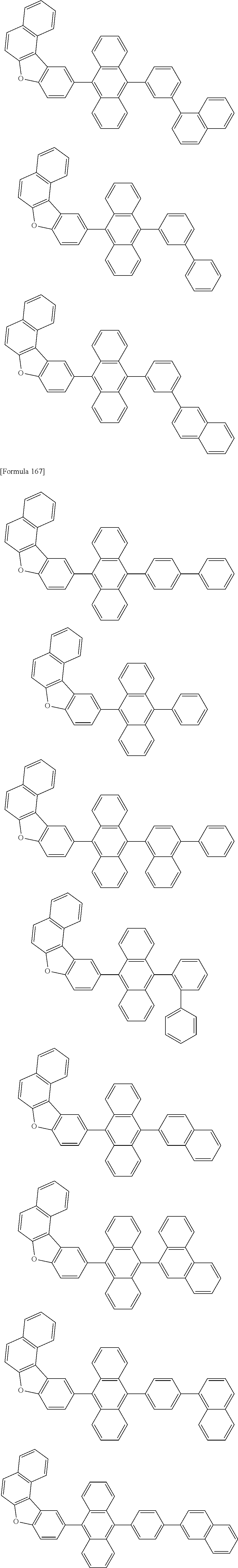

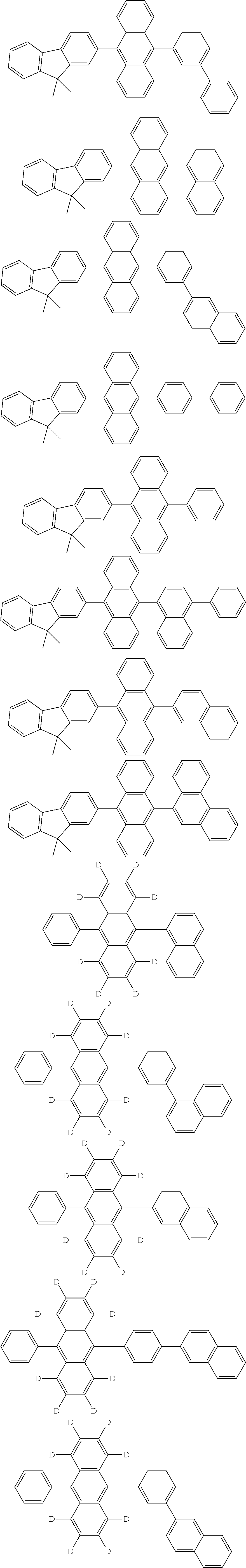

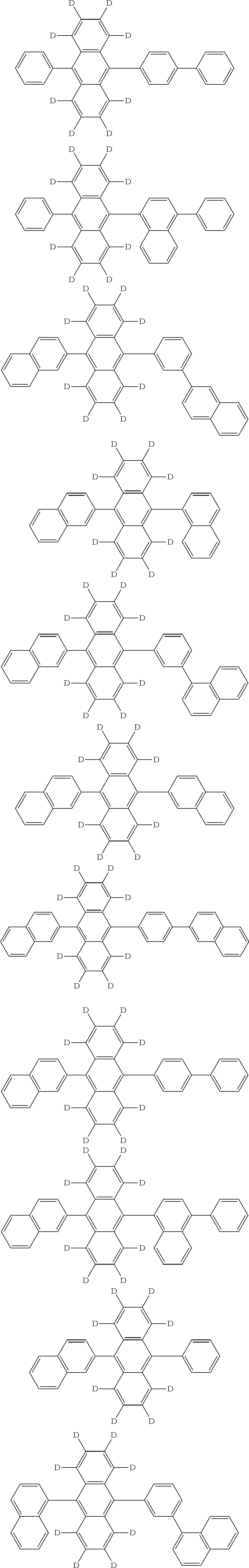

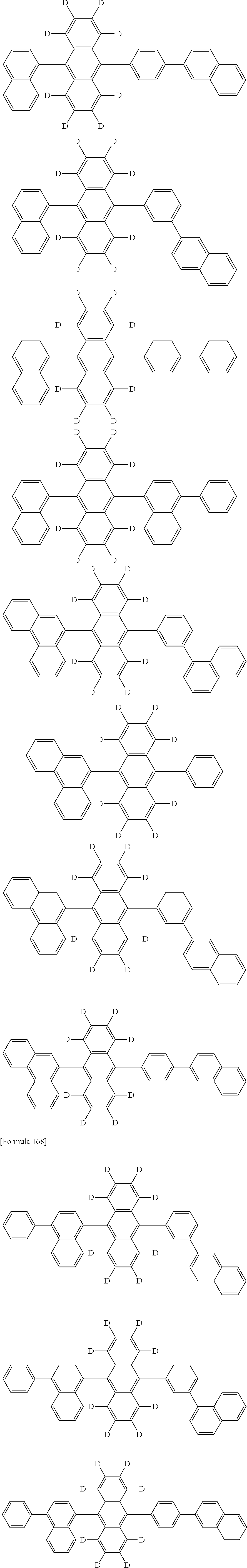

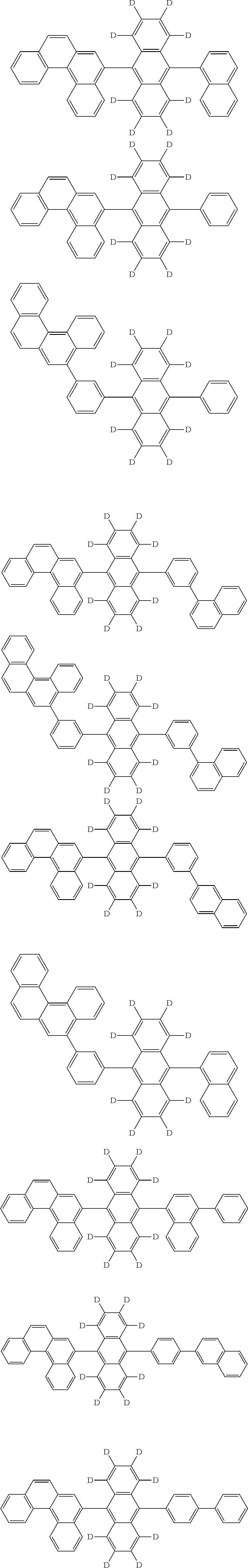

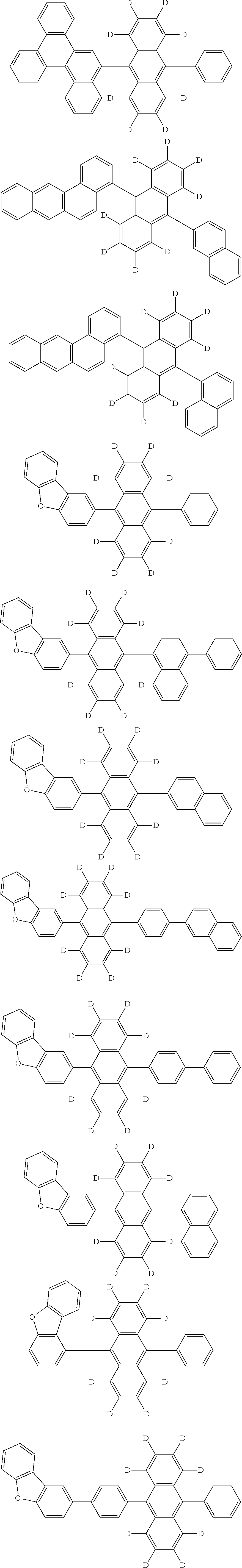

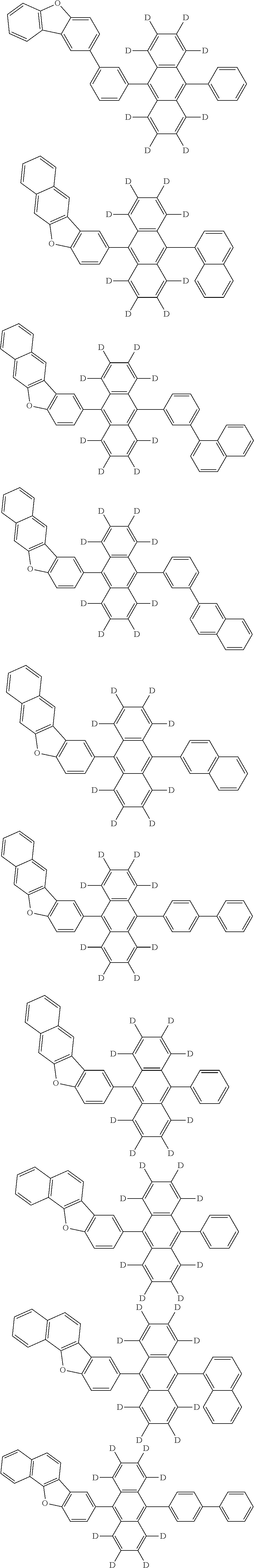

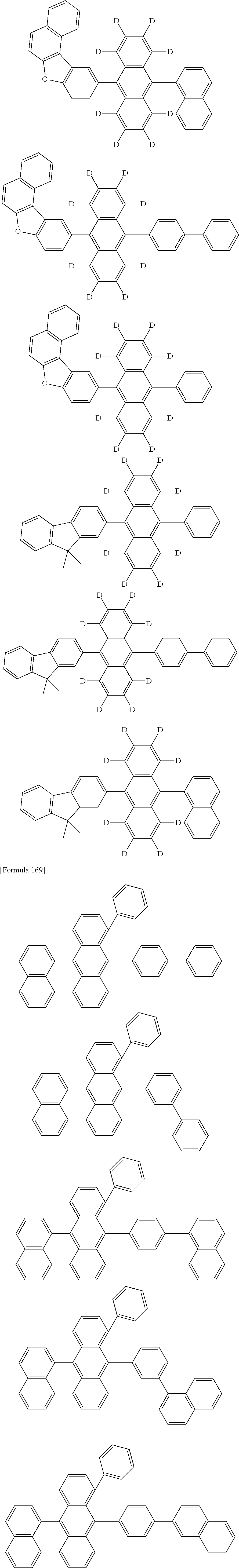

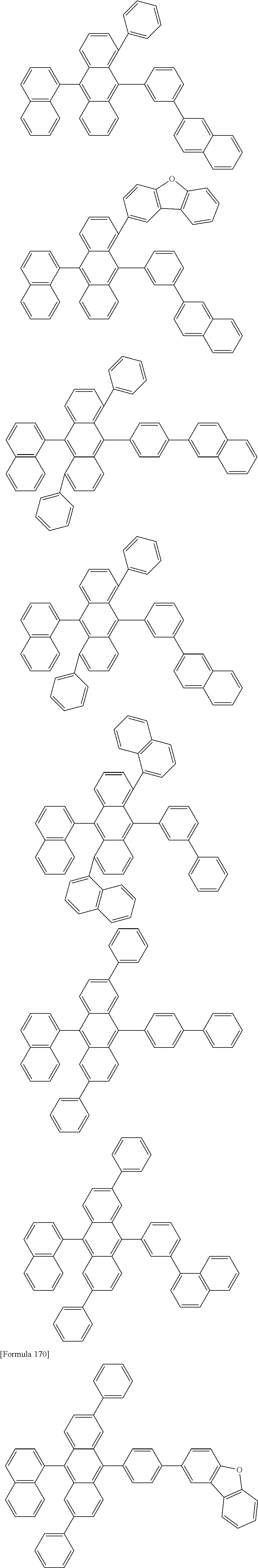

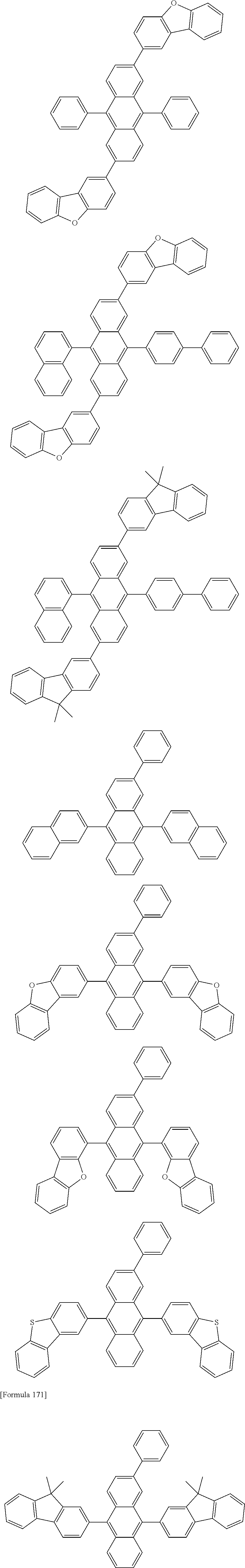

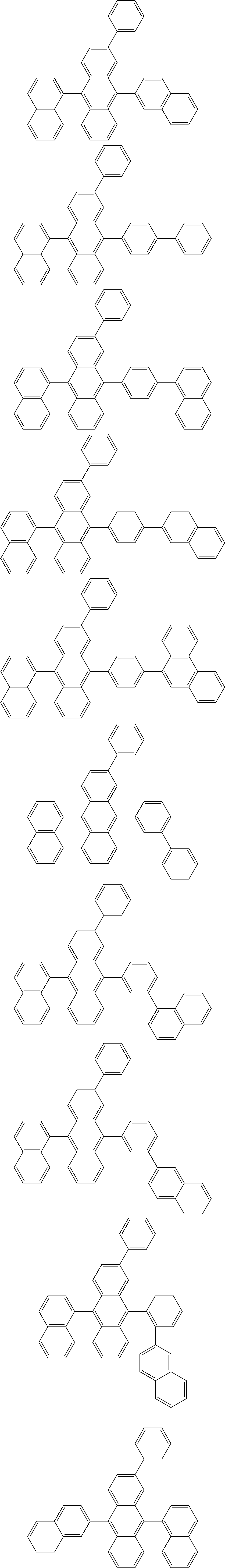

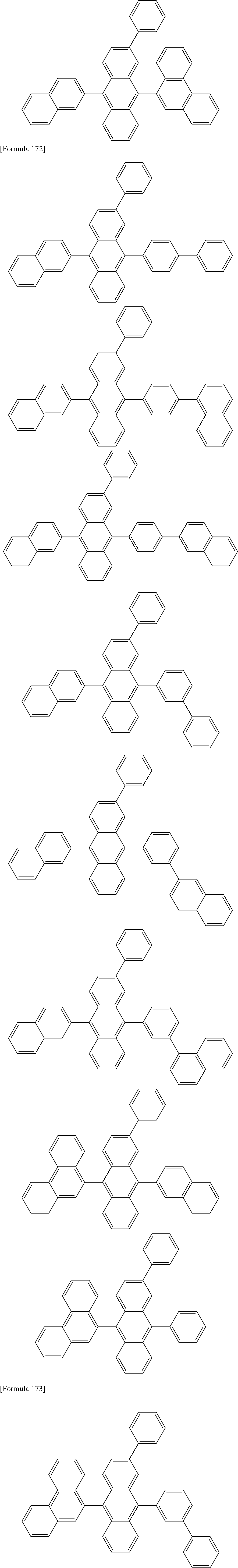

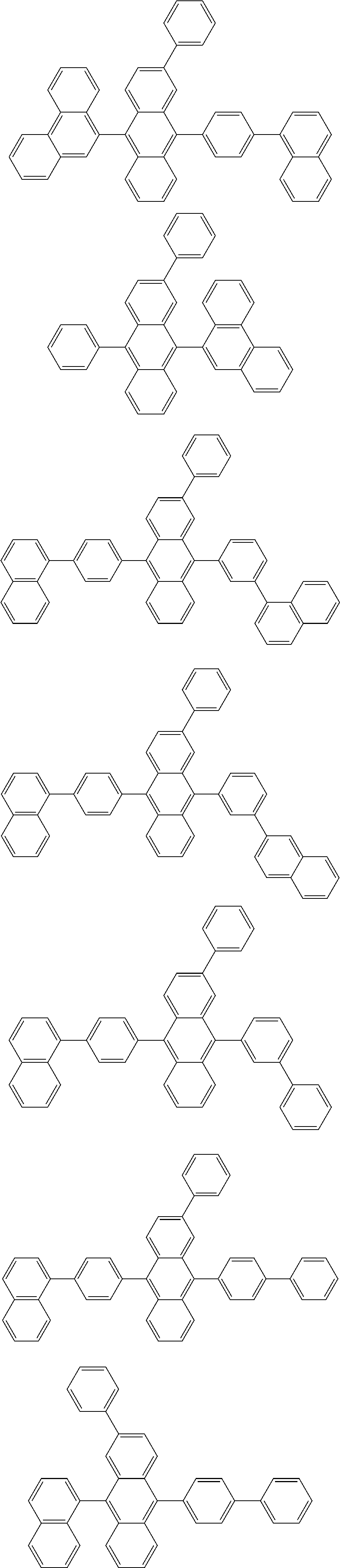

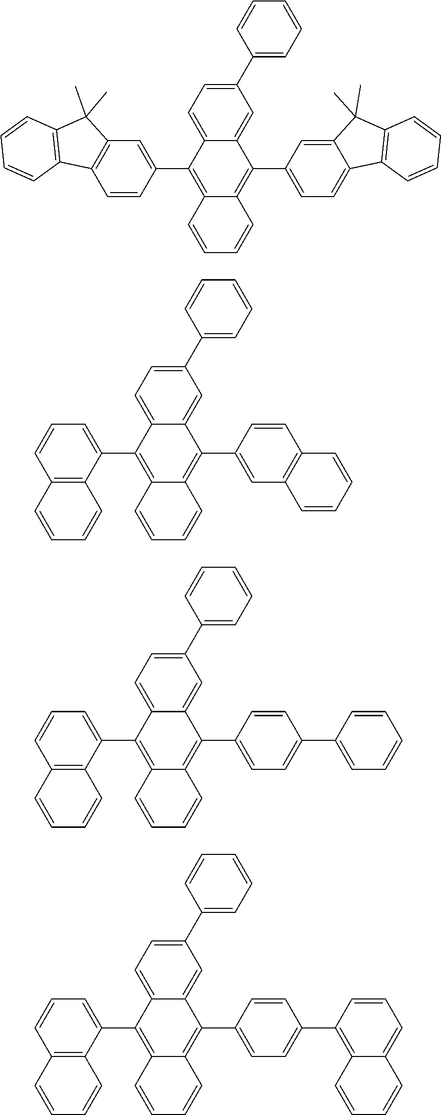

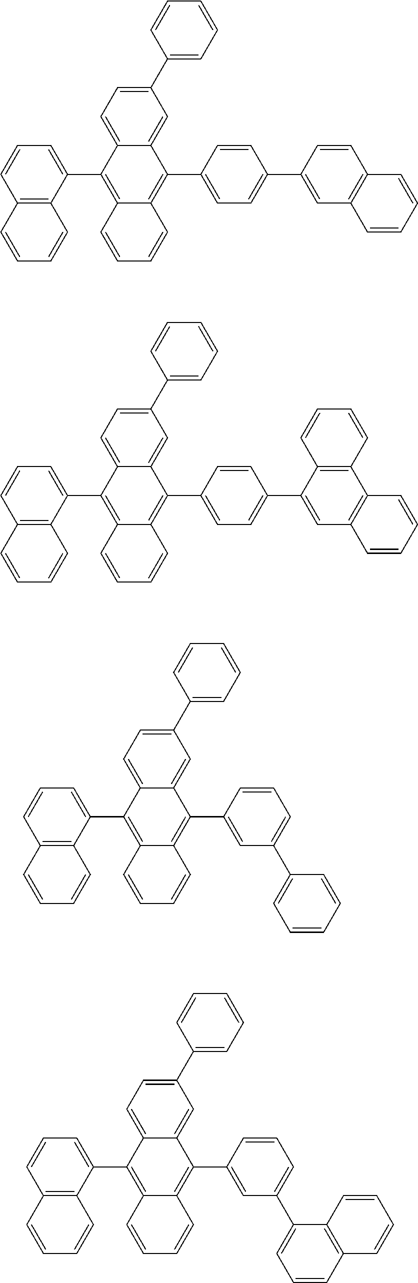

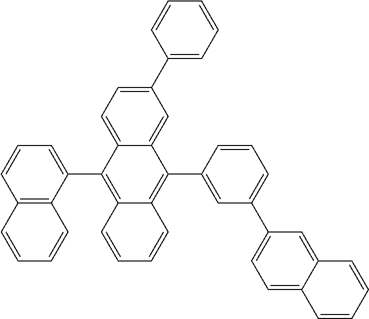 [SMILES: c1ccc(-c2ccc3c(-c4cccc5ccccc45)c4ccccc4c(-c4cccc(-c5ccc6ccccc6c5)c4)c3c2)cc1]

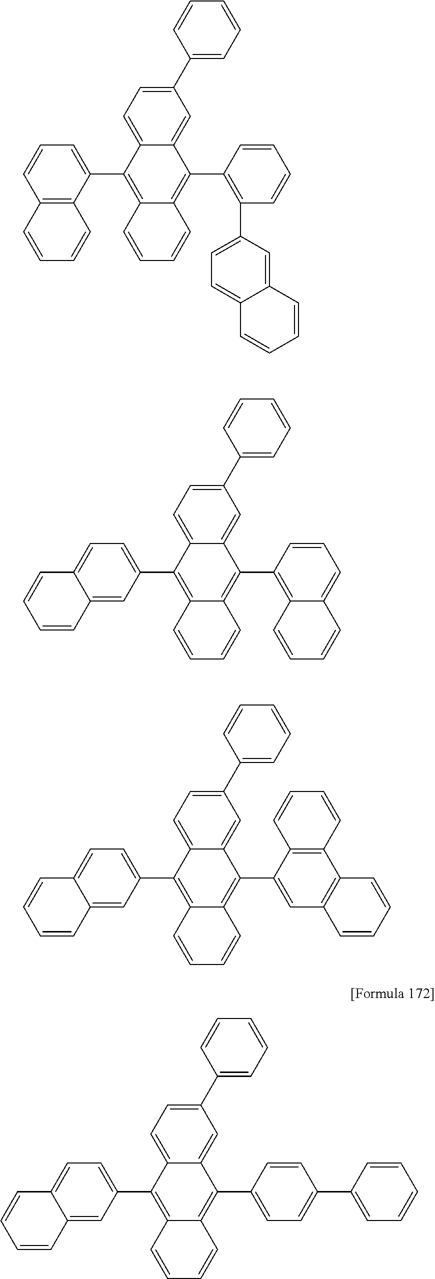

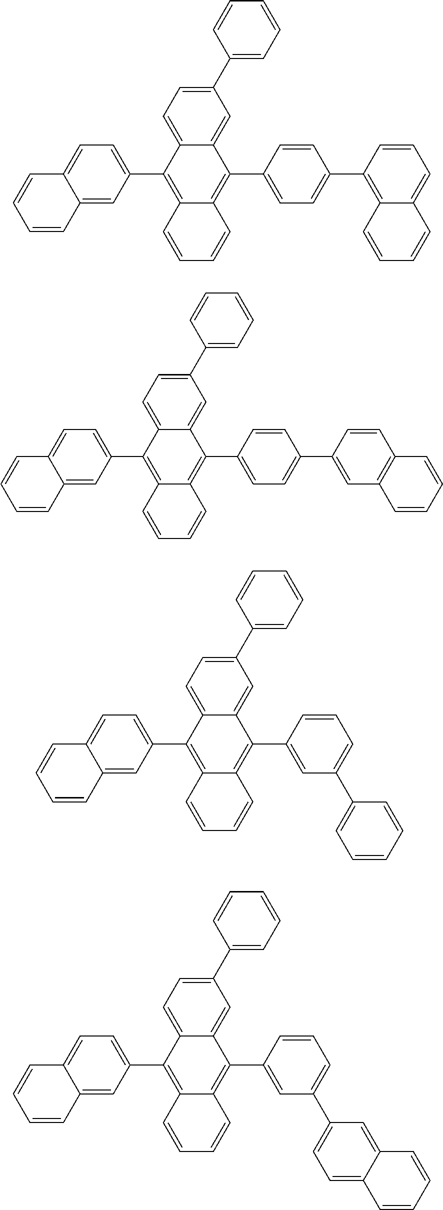

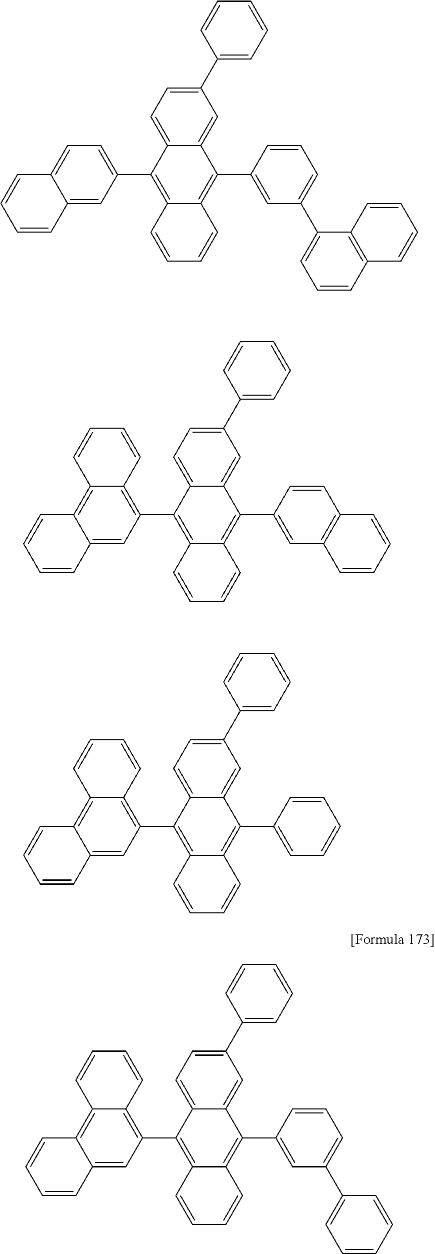

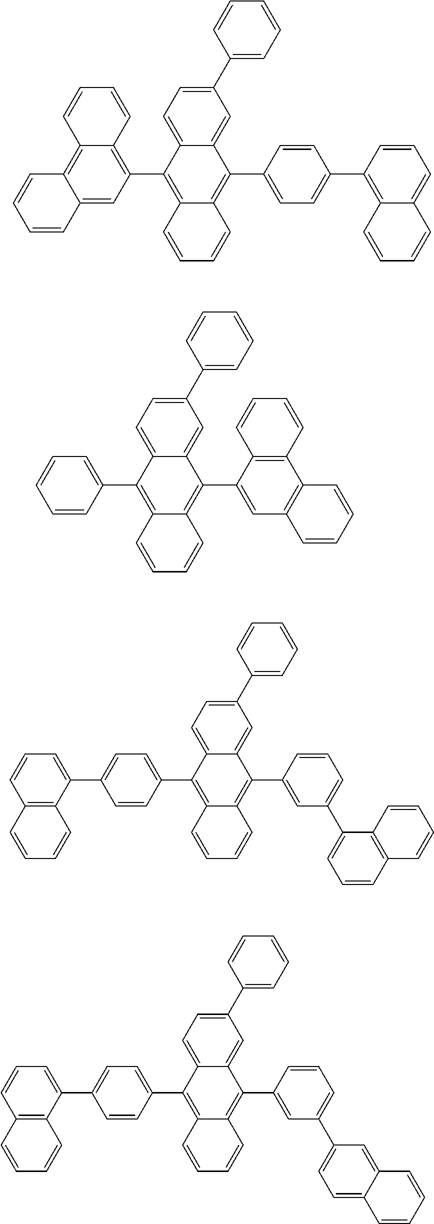

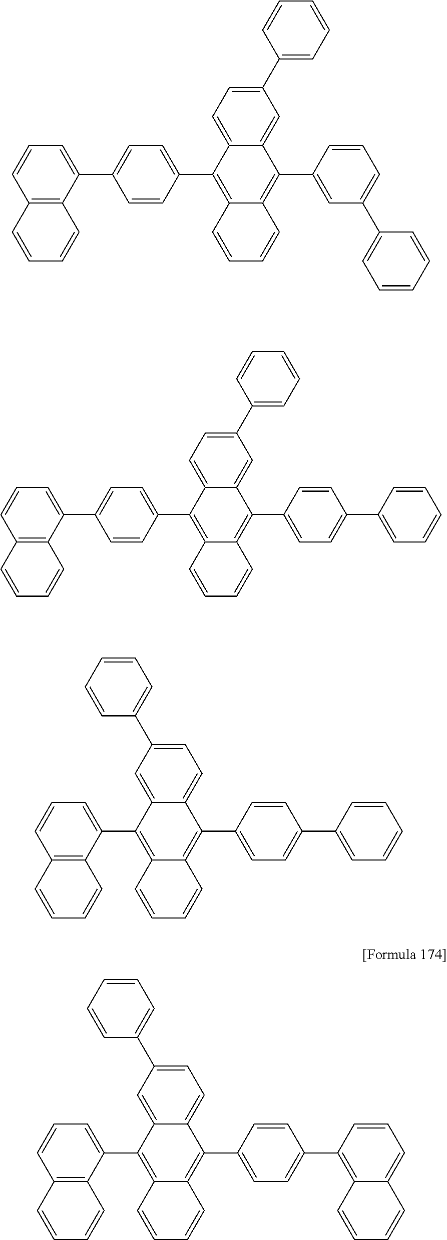

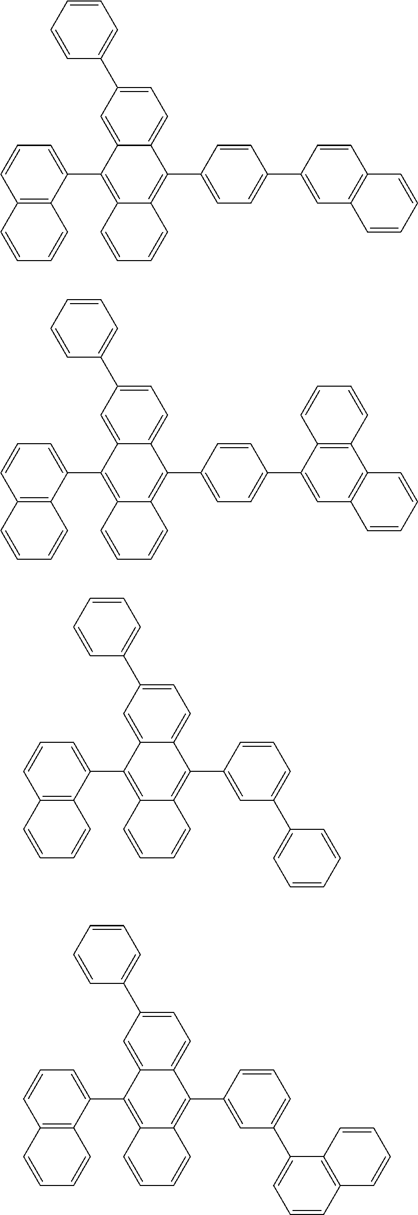

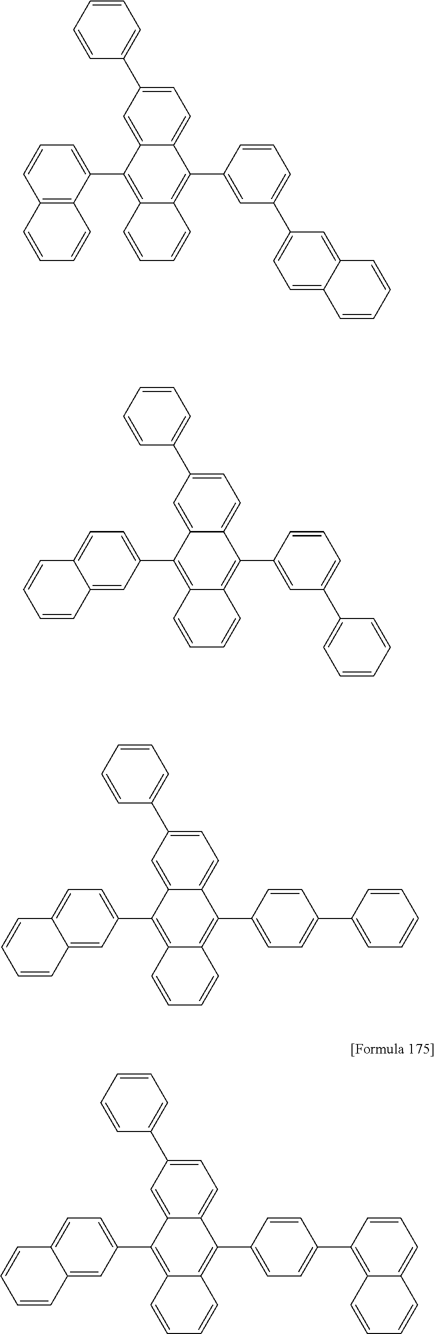

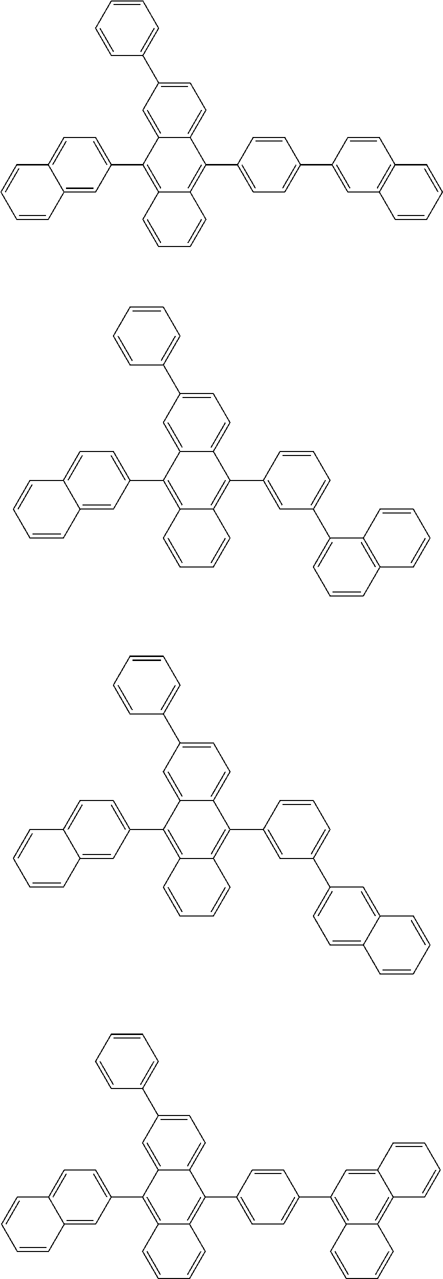

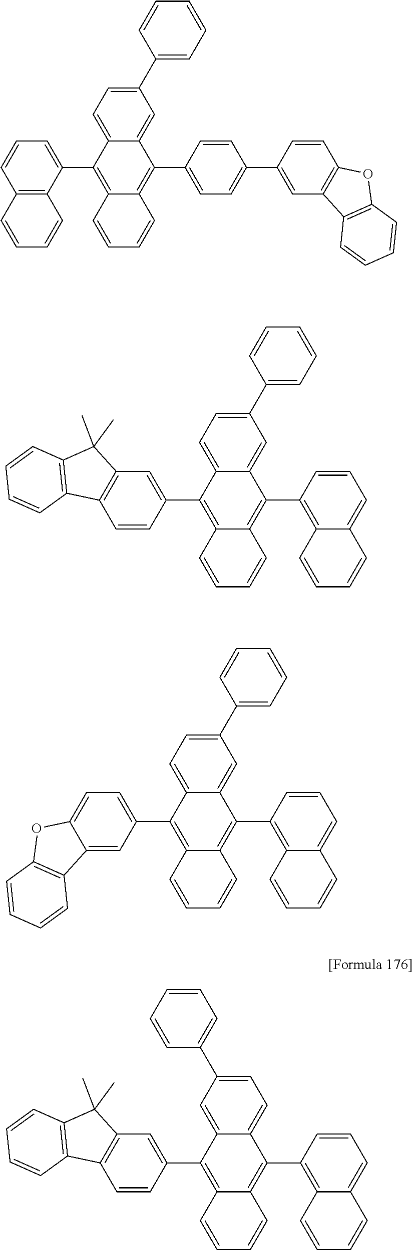

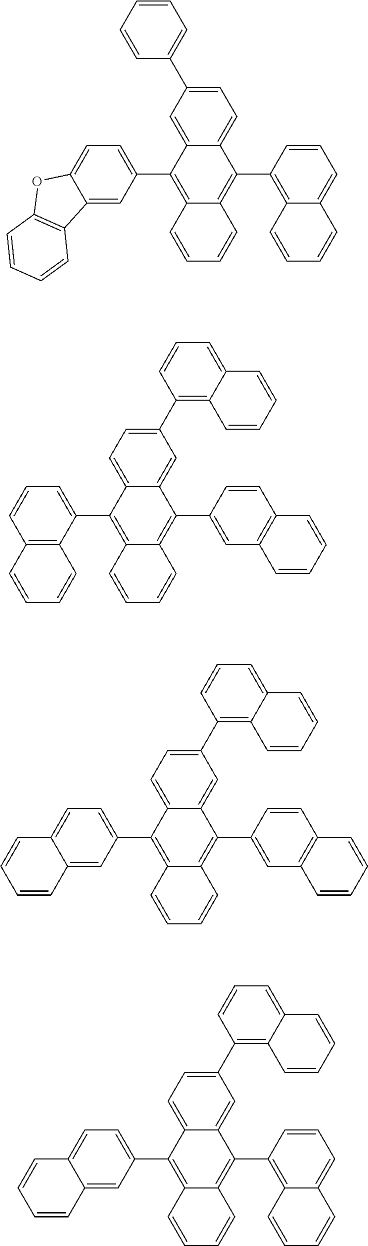

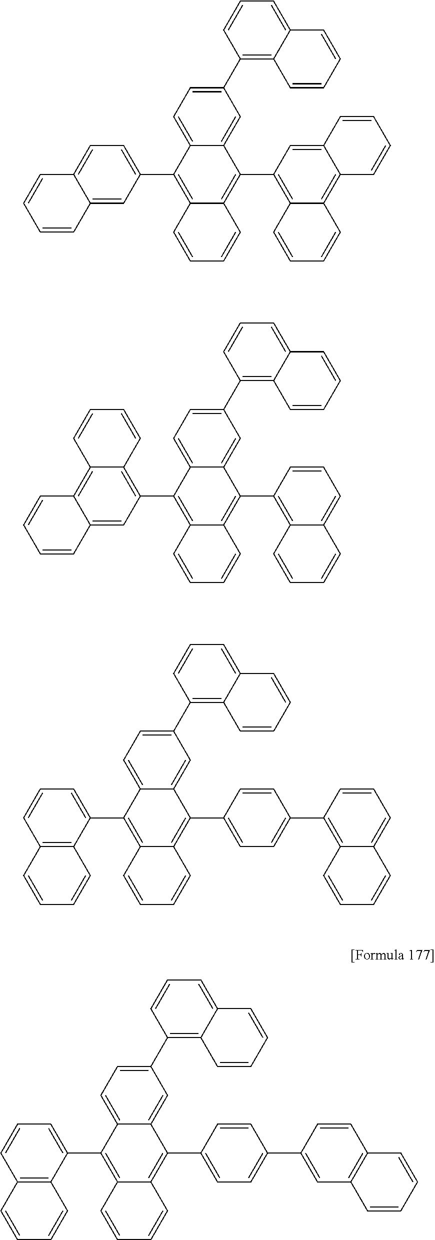

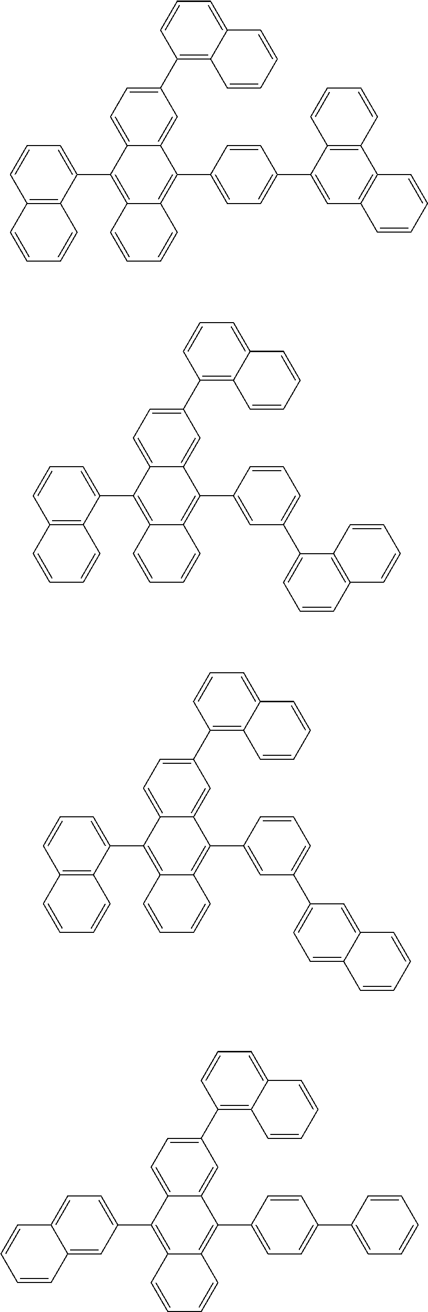

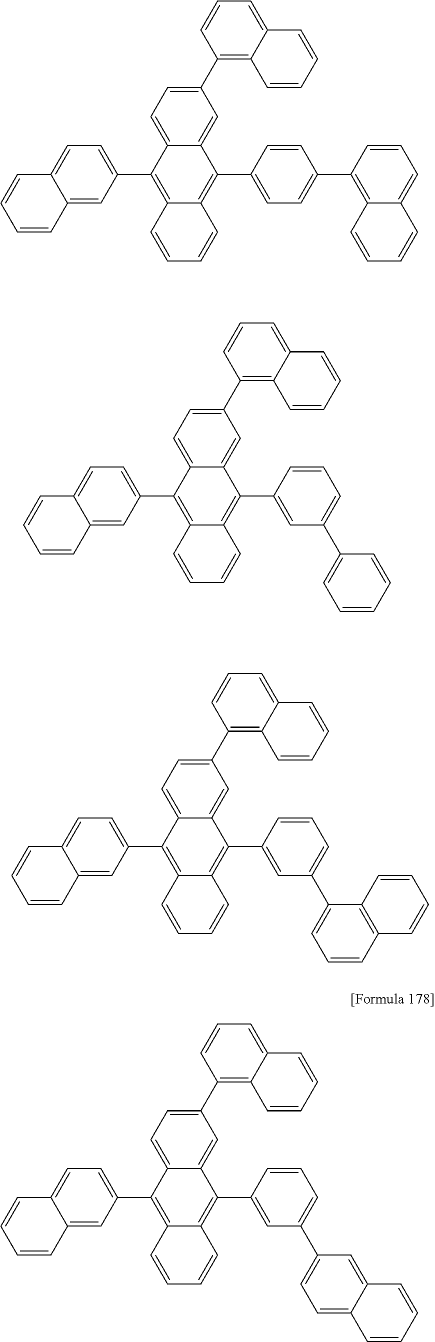

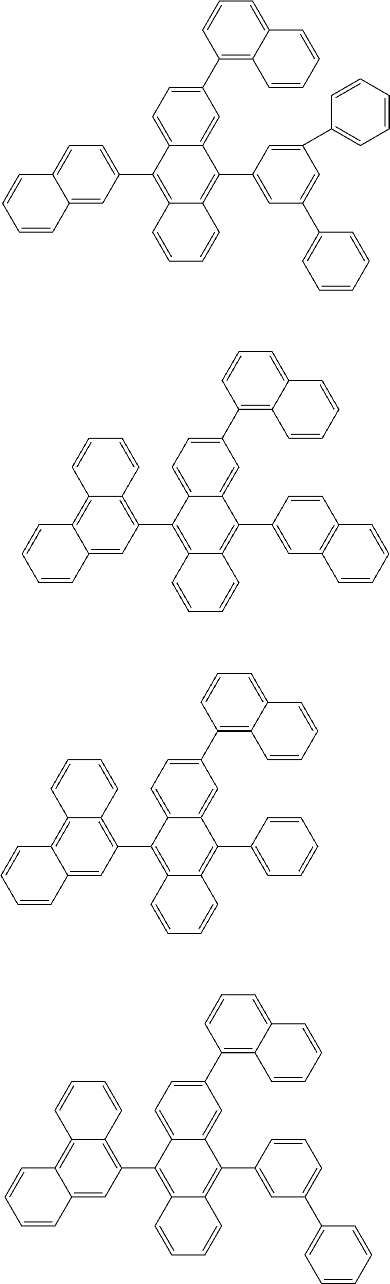

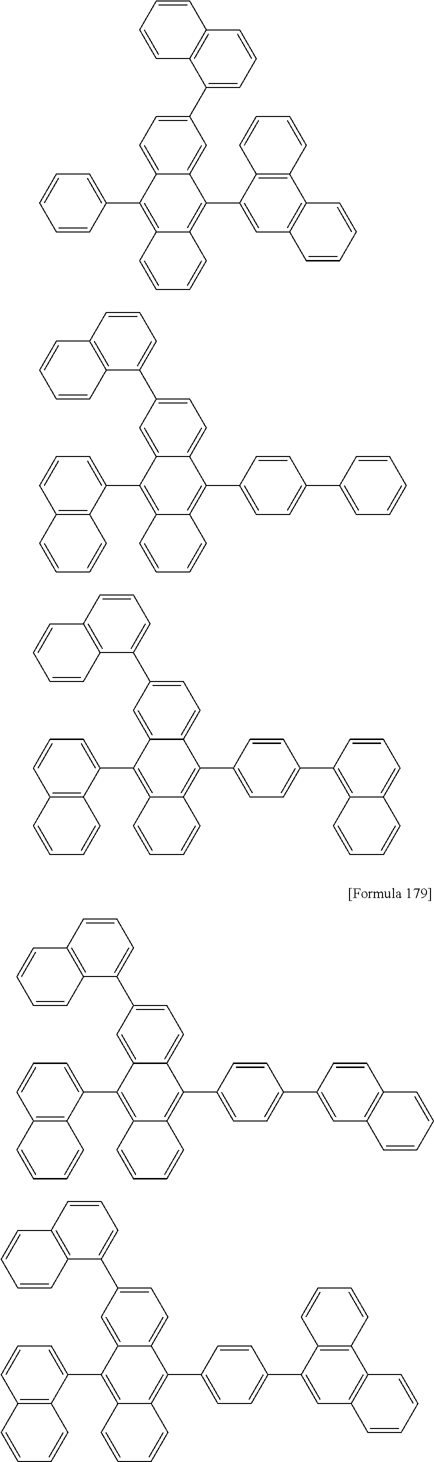

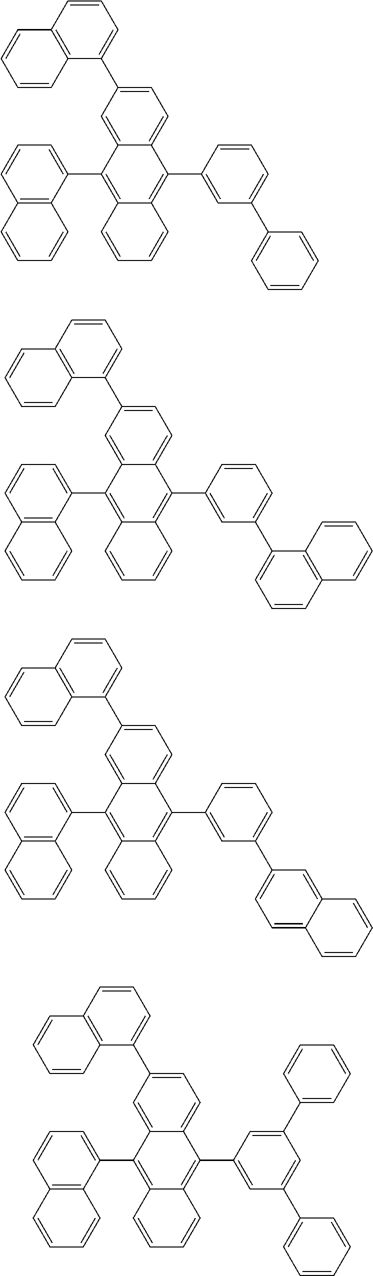

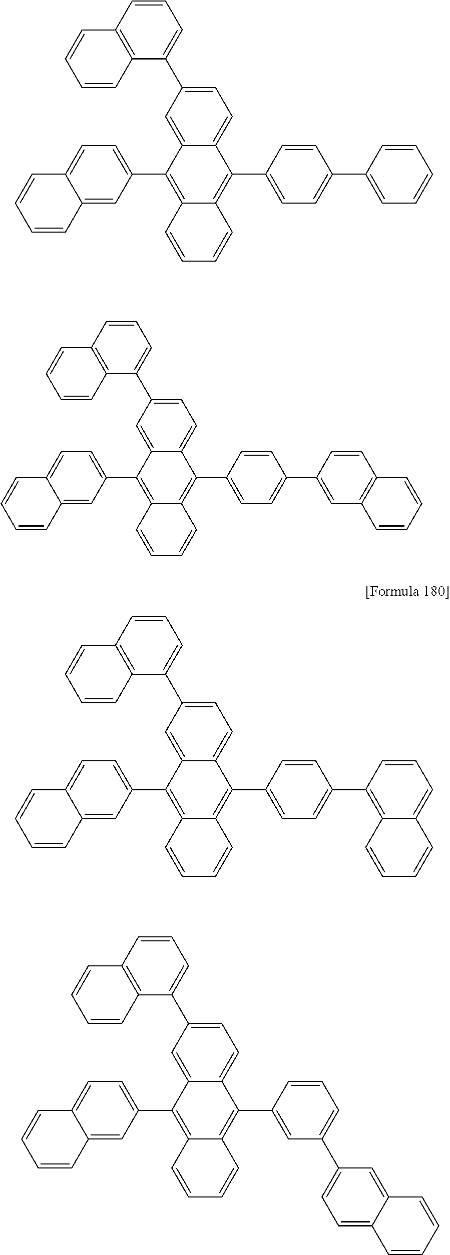

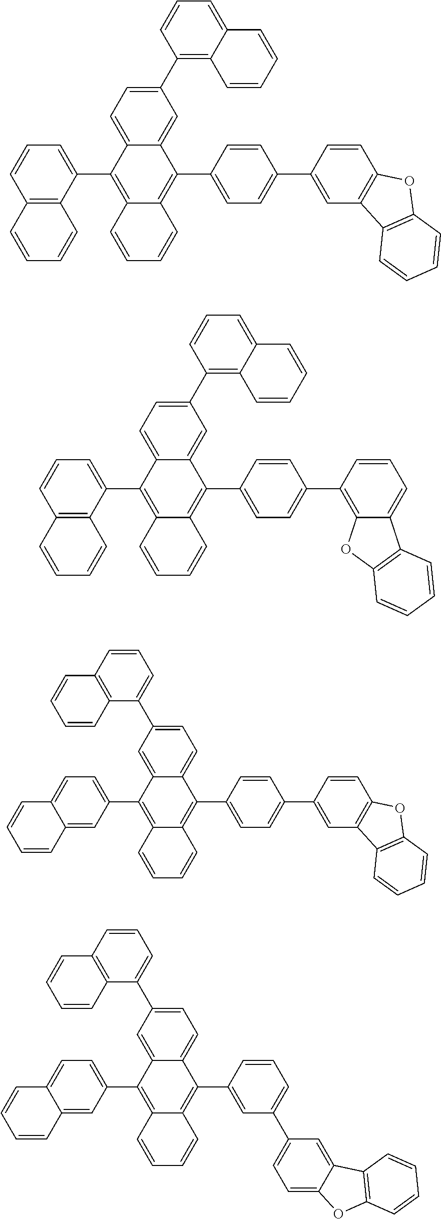 [SMILES: c1ccc2c(-c3ccc4c(-c5cccc6ccccc56)c5ccccc5c(-c5ccc(-c6ccc7oc8ccccc8c7c6)cc5)c4c3)cccc2c1 ; c1ccc2c(-c3ccc4c(-c5cccc6ccccc56)c5ccccc5c(-c5ccc(-c6cccc7c6oc6ccccc67)cc5)c4c3)cccc2c1 ; c1ccc2cc(-c3c4ccccc4c(-c4ccc(-c5ccc6oc7ccccc7c6c5)cc4)c4ccc(-c5cccc6ccccc56)cc34)ccc2c1 ; c1cc(-c2ccc3oc4ccccc4c3c2)cc(-c2c3ccccc3c(-c3ccc4ccccc4c3)c3cc(-c4cccc5ccccc45)ccc23)c1]

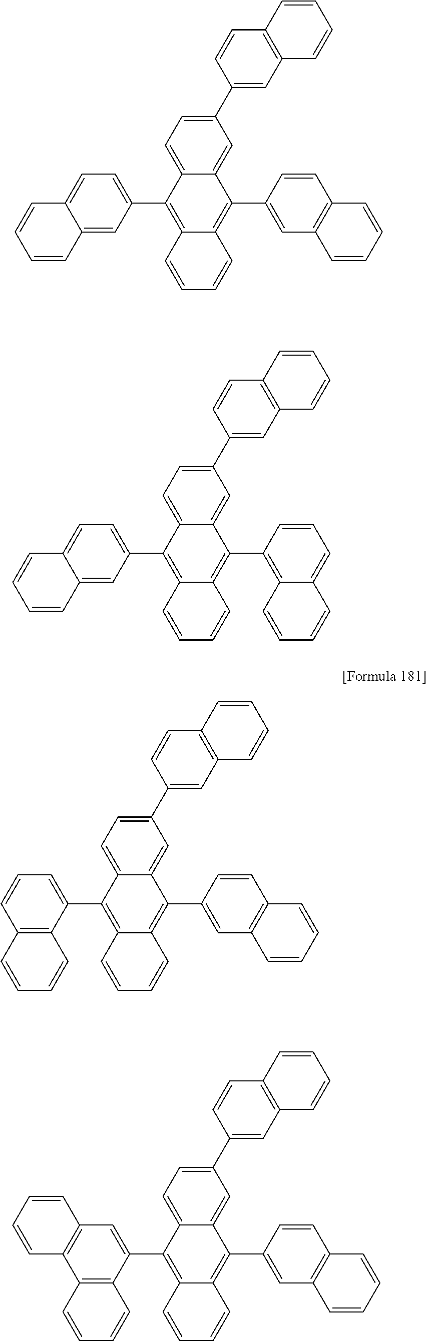

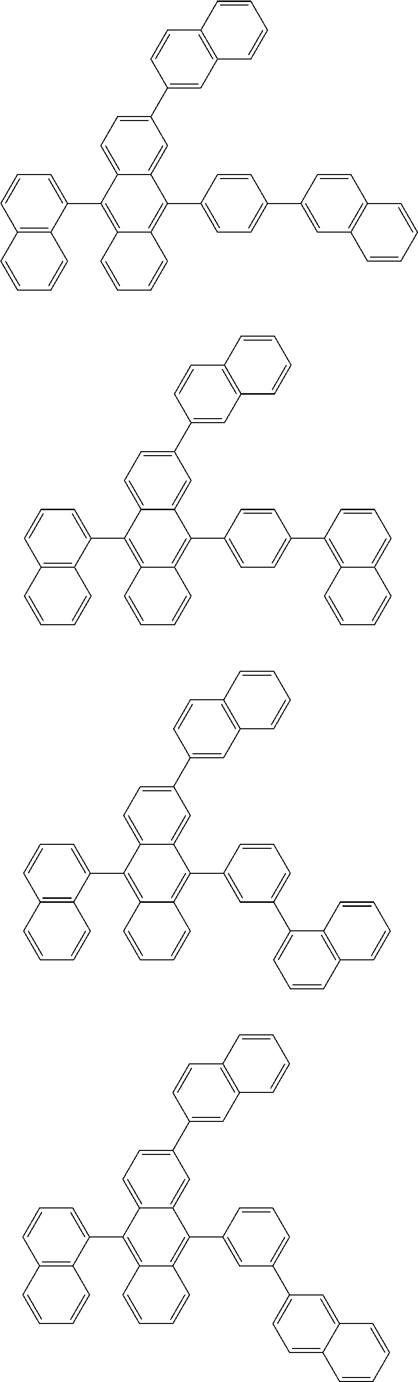

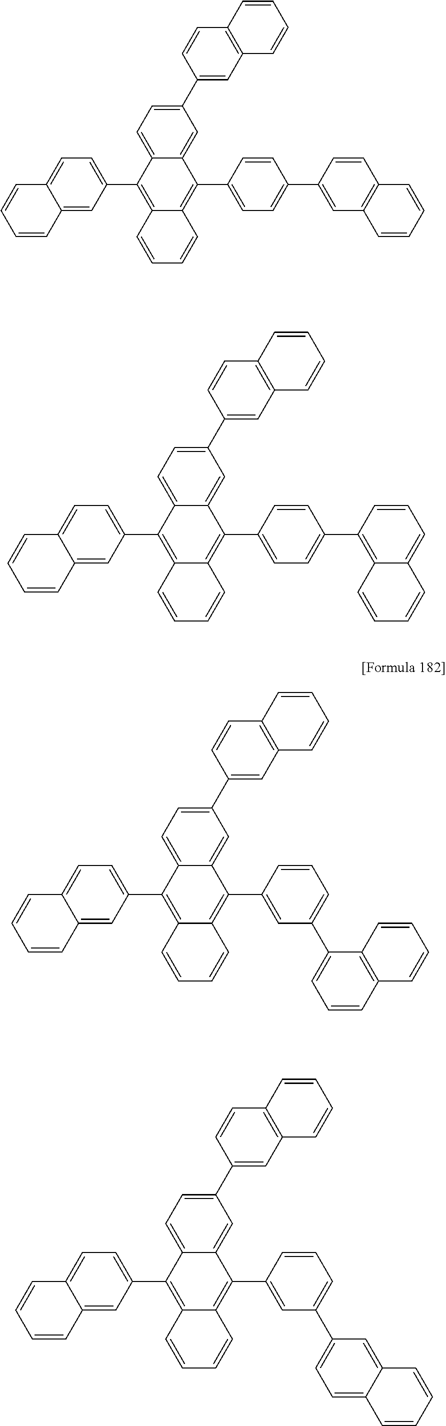

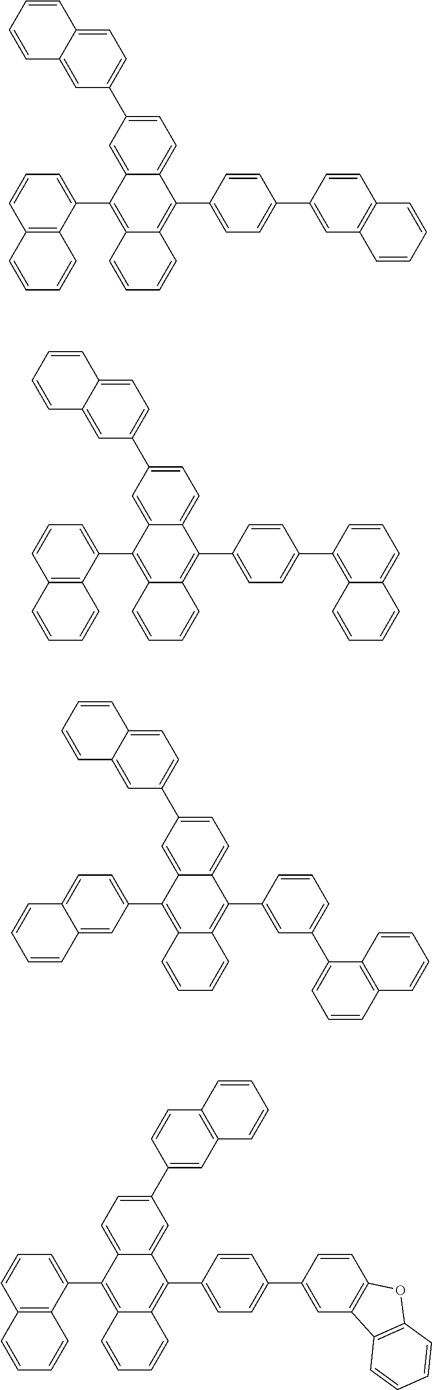

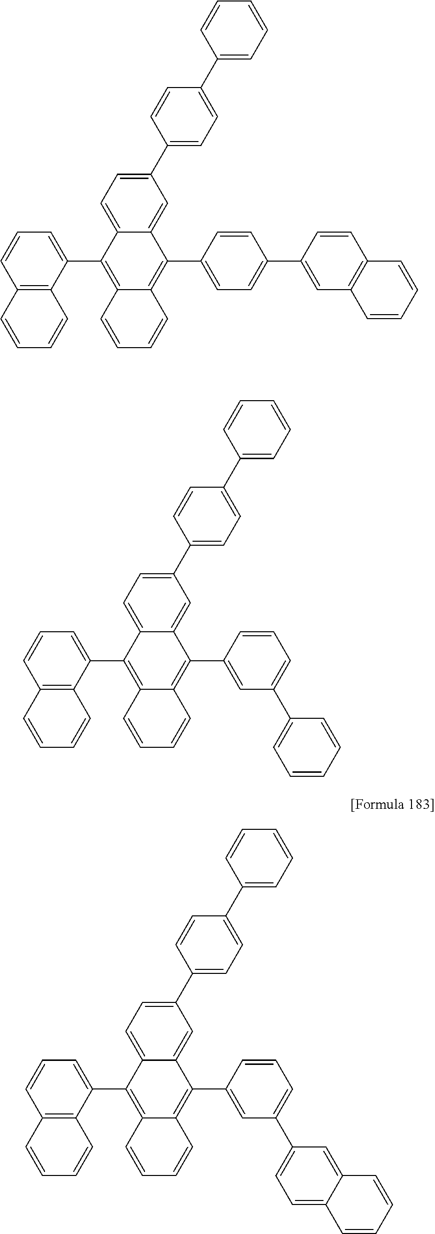

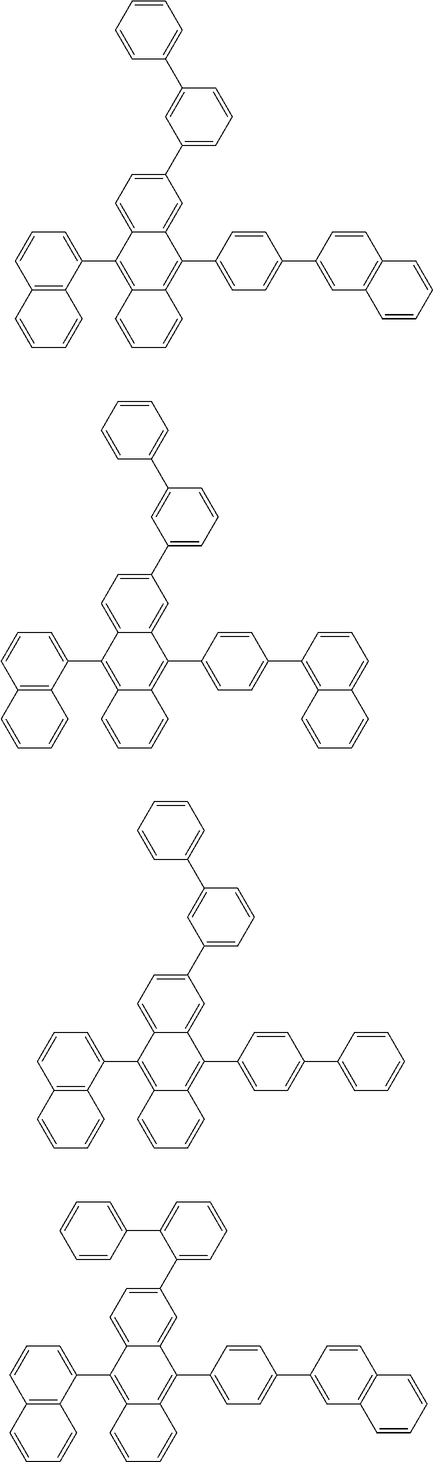

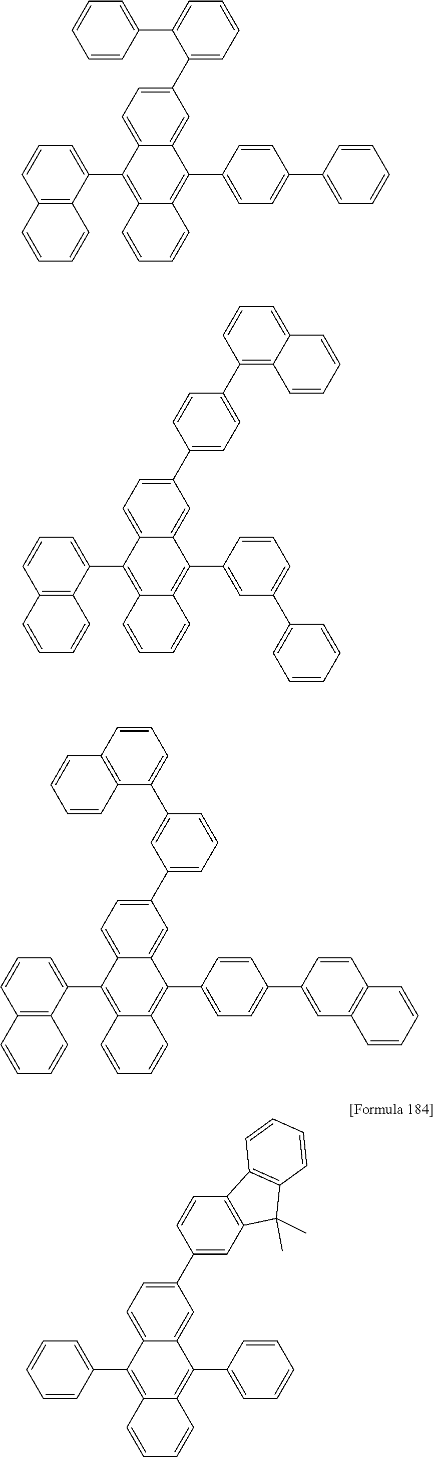

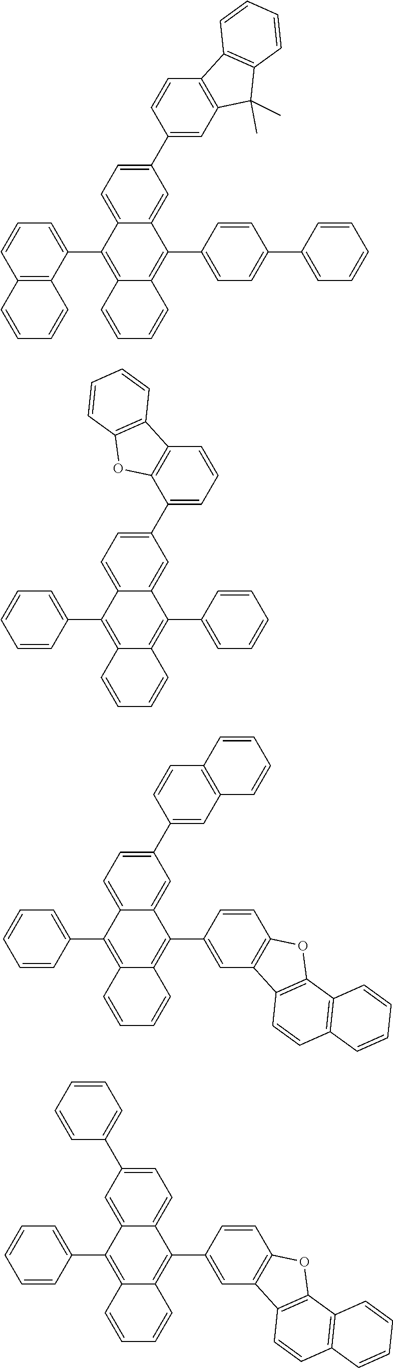

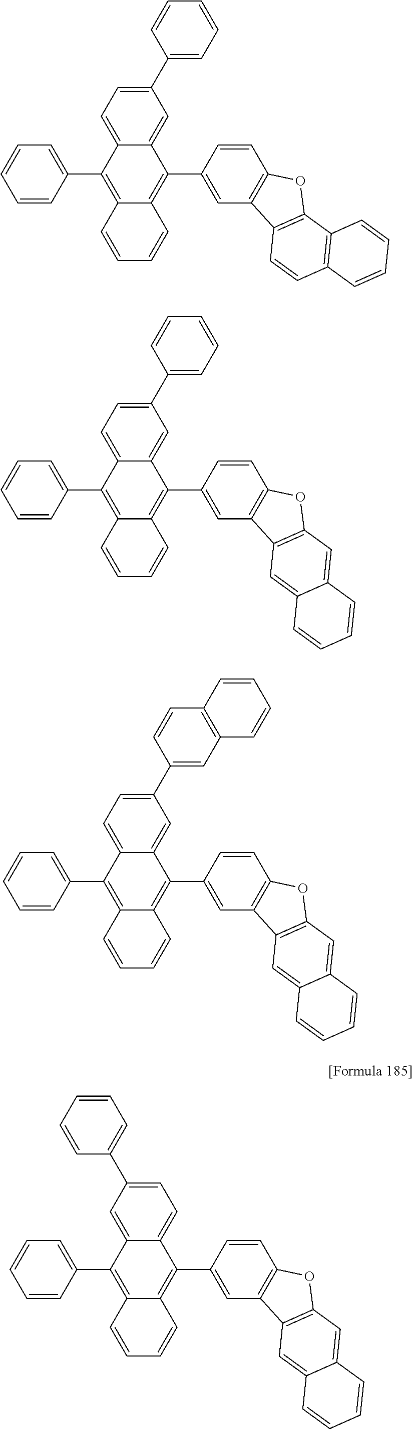 [SMILES: c1ccc(-c2ccc3c(-c4ccccc4)c4ccccc4c(-c4ccc5oc6c7ccccc7ccc6c5c4)c3c2)cc1 ; c1ccc(-c2ccc3c(-c4ccccc4)c4ccccc4c(-c4ccc5oc6cc7ccccc7cc6c5c4)c3c2)cc1 ; c1ccc(-c2c3ccccc3c(-c3ccc4oc5cc6ccccc6cc5c4c3)c3cc(-c4ccc5ccccc5c4)ccc23)cc1 ; c1ccc(-c2ccc3c(-c4ccc5oc6cc7ccccc7cc6c5c4)c4ccccc4c(-c4ccccc4)c3c2)cc1]

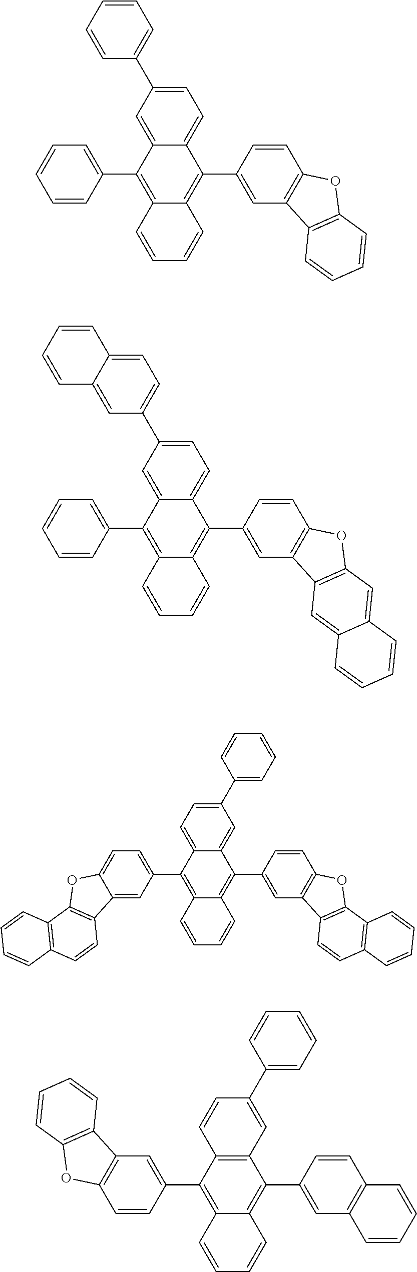 [SMILES: c1ccc(-c2ccc3c(-c4ccc5oc6ccccc6c5c4)c4ccccc4c(-c4ccccc4)c3c2)cc1 ; c1ccc(-c2c3ccccc3c(-c3ccc4oc5cc6ccccc6cc5c4c3)c3ccc(-c4ccc5ccccc5c4)cc23)cc1 ; c1ccc(-c2ccc3c(-c4ccc5oc6c7ccccc7ccc6c5c4)c4ccccc4c(-c4ccc5oc6c7ccccc7ccc6c5c4)c3c2)cc1 ; c1ccc(-c2ccc3c(-c4ccc5oc6ccccc6c5c4)c4ccccc4c(-c4ccc5ccccc5c4)c3c2)cc1]

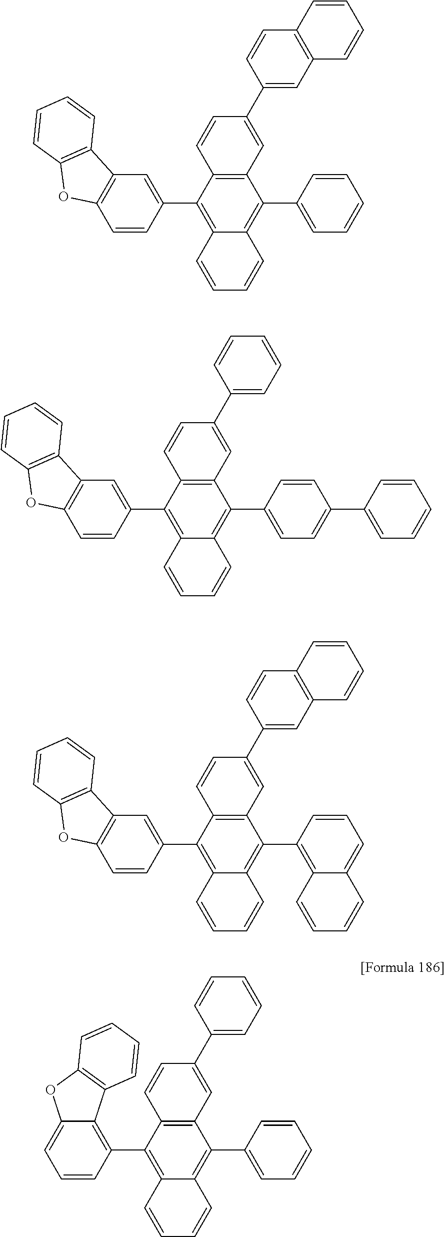

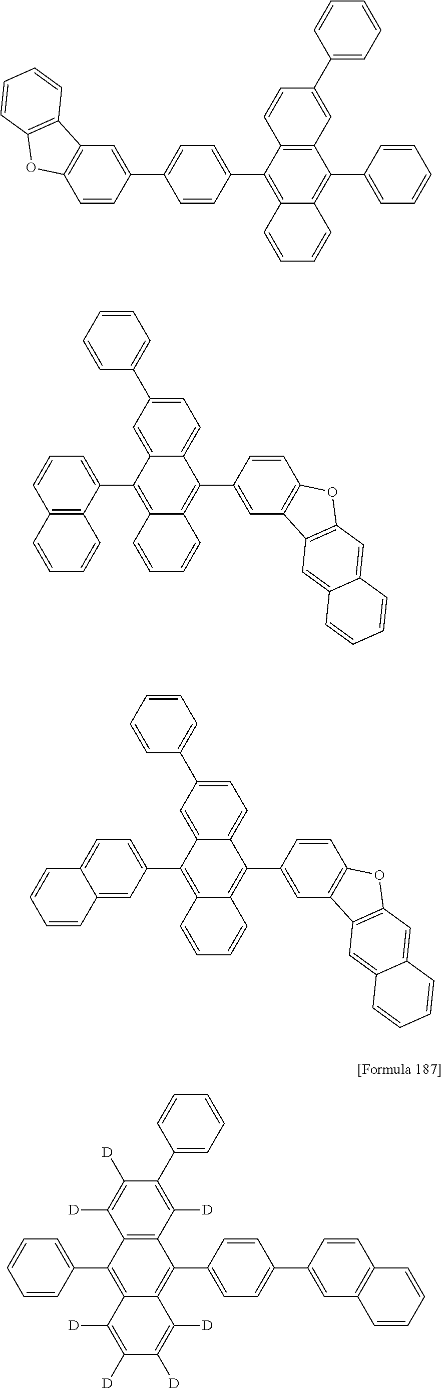 [SMILES: c1ccc(-c2ccc3c(-c4ccc(-c5ccc6oc7ccccc7c6c5)cc4)c4ccccc4c(-c4ccccc4)c3c2)cc1 ; c1ccc(-c2ccc3c(-c4ccc5oc6cc7ccccc7cc6c5c4)c4ccccc4c(-c4cccc5ccccc45)c3c2)cc1 ; c1ccc(-c2ccc3c(-c4ccc5oc6cc7ccccc7cc6c5c4)c4ccccc4c(-c4ccc5ccccc5c4)c3c2)cc1 ; [2H]c1c([2H])c([2H])c2c(-c3ccc(-c4ccc5ccccc5c4)cc3)c3c([2H])c(-c4ccccc4)c([2H])c([2H])c3c(-c3ccccc3)c2c1[2H]]

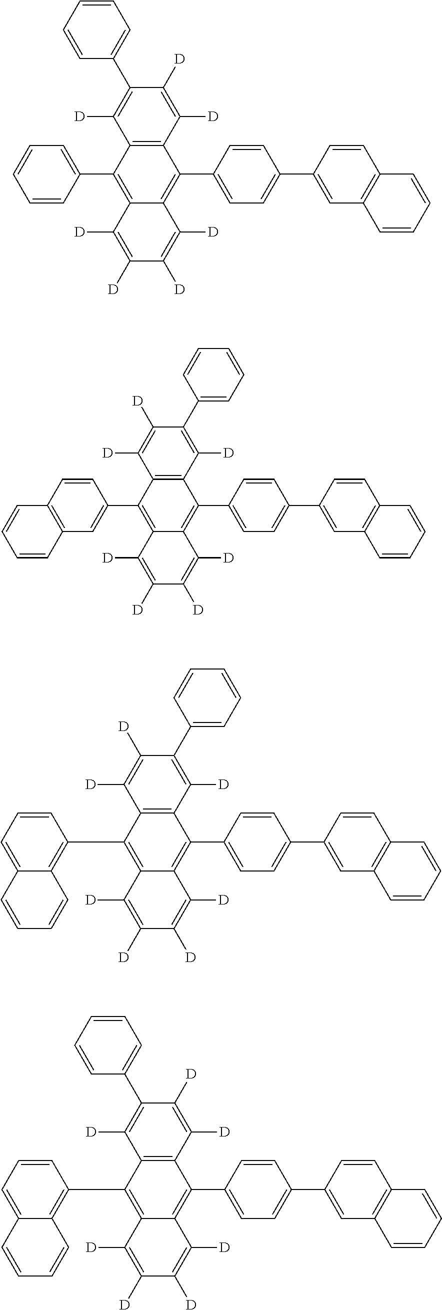 [SMILES: [2H]c1c([2H])c([2H])c2c(-c3ccccc3)c3c([2H])c(-c4ccccc4)c([2H])c([2H])c3c(-c3ccc(-c4ccc5ccccc5c4)cc3)c2c1[2H] ; [2H]c1c([2H])c([2H])c2c(-c3ccc(-c4ccc5ccccc5c4)cc3)c3c([2H])c(-c4ccccc4)c([2H])c([2H])c3c(-c3ccc4ccccc4c3)c2c1[2H] ; [2H]c1c([2H])c([2H])c2c(-c3cccc4ccccc34)c3c([2H])c([2H])c(-c4ccccc4)c([2H])c3c(-c3ccc(-c4ccc5ccccc5c4)cc3)c2c1[2H] ; [2H]c1c([2H])c([2H])c2c(-c3cccc4ccccc34)c3c([2H])c(-c4ccccc4)c([2H])c([2H])c3c(-c3ccc(-c4ccc5ccccc5c4)cc3)c2c1[2H]]

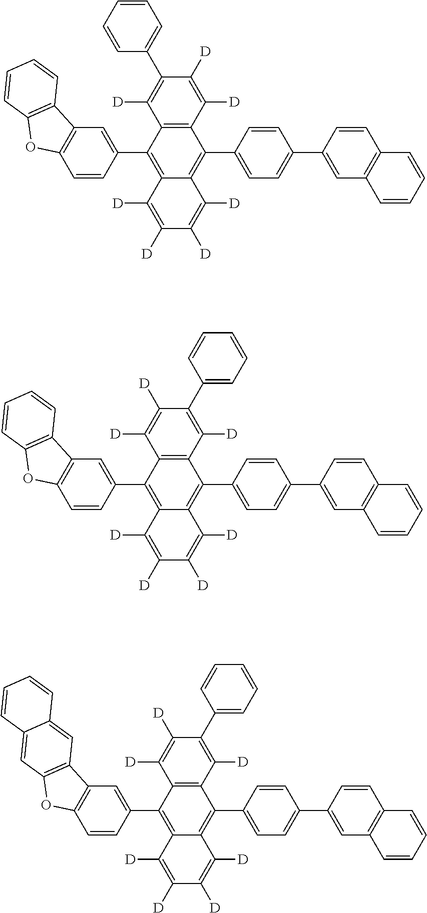 [SMILES: [2H]c1c([2H])c([2H])c2c(-c3ccc4oc5ccccc5c4c3)c3c([2H])c(-c4ccccc4)c([2H])c([2H])c3c(-c3ccc(-c4ccc5ccccc5c4)cc3)c2c1[2H] ; [2H]c1c([2H])c([2H])c2c(-c3ccc(-c4ccc5ccccc5c4)cc3)c3c([2H])c(-c4ccccc4)c([2H])c([2H])c3c(-c3ccc4oc5ccccc5c4c3)c2c1[2H] ; [2H]c1c([2H])c([2H])c2c(-c3ccc(-c4ccc5ccccc5c4)cc3)c3c([2H])c(-c4ccccc4)c([2H])c([2H])c3c(-c3ccc4oc5cc6ccccc6cc5c4c3)c2c1[2H]]

### First Dopant Material, Second Dopant Material, and Third Dopant Material

In the organic EL device of the exemplary embodiment, the first dopant material, the second dopant material, and the third dopant material are exemplified by a third compound and a fourth compound.

The third compound and the fourth compound are each independently at least one compound selected from the group consisting of a compound represented by a formula (3) below, a compound represented by a formula (4) below, a compound represented by a formula (5) below, a compound represented by a formula (6) below, a compound represented by a formula (7) below, a compound represented by a formula (8) below, the compound represented by the formula (9), and a compound represented by a formula (10) below.

### Compound Represented by Formula (3)

A compound represented by the formula (3) will be described.

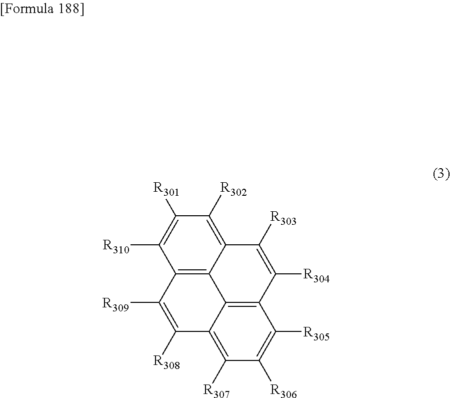

In the formula (3):
at least one combination of adjacent two or more of R₃₀₁ to R₃₁₀ are mutually bonded to form a substituted or unsubstituted monocyclic ring, mutually bonded to form a substituted or unsubstituted fused ring, or not mutually bonded;
at least one of R₃₀₁ to R₃₁₀ is a monovalent group represented by a formula (31) below; and
R₃₀₁ to R₃₁₀ forming neither the monocyclic ring nor the fused ring and not being the monovalent group represented by the formula (31) are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

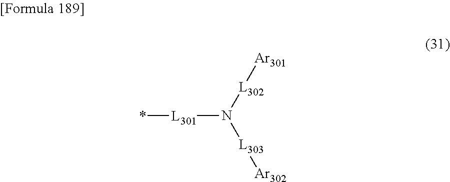

In the formula (31):
Ar₃₀₁ and Ar₃₀₂ are each independently a substituted or unsubstituted aryl group having 6 to 50 ring carbon atoms, or a substituted or unsubstituted heterocyclic group having 5 to 50 ring atoms;
L₃₀₁ to L₃₀₃ are each independently a single bond, a substituted or unsubstituted arylene group having 6 to 30 ring carbon atoms, or a substituted or unsubstituted divalent heterocyclic group having 5 to 30 ring atoms; and
* represents a bonding position with a pyrene ring in the formula (3).

In the third compound and the fourth compound, R₉₀₁, R₉₀₂, R₉₀₃, R₉₀₄, R₉₀₅, R₉₀₆, and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

Two of R₃₀₁ to R₃₁₀ in the formula (3) are preferably a group represented by the formula (31).

In an exemplary embodiment, a compound represented by the formula (3) is a compound represented by a formula (33) below.

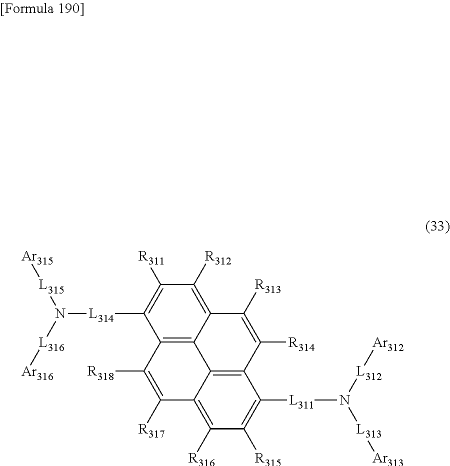

In the formula (33):
R₃₁₁ to R₃₁₈ each independently represent the same as R₃₀₁ to R₃₁₀ in the formula (3) that are not a monovalent group represented by the formula (31);
L₃₁₁ to L₃₁₆ are each independently a single bond, a substituted or unsubstituted arylene group having 6 to 30 ring carbon atoms, or a substituted or unsubstituted divalent heterocyclic group having 5 to 30 ring atoms; and
Ar₃₁₂, Ar₃₁₃, Ar₃₁₅, and Ar₃₁₆ are each independently a substituted or unsubstituted aryl group having 6 to 50 ring carbon atoms, or a substituted or unsubstituted heterocyclic group having 5 to 50 ring atoms.

In the formula (31), L₃₀₁ is preferably a single bond, and L₃₀₂ and L₃₀₃ are preferably a single bond.

In an exemplary embodiment, a compound represented by the formula (3) is represented by a formula (34) or a formula (35) below.

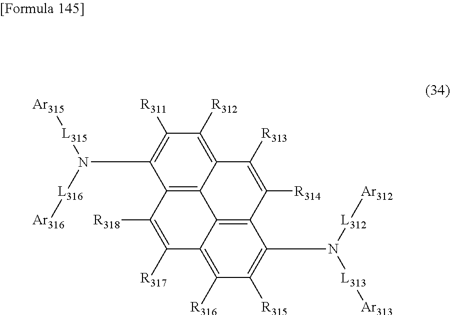

In the formula (34):
R₃₁₁ to R₃₁₈ each independently represent the same as R₃₀₁ to R₃₁₀ in the formula (3) that are not a monovalent group represented by the formula (31);
L₃₁₂, L₃₁₃, L₃₁₅, and L₃₁₆ each independently represent the same as L₃₁₂, L₃₁₃, L₃₁₅, and L₃₁₆ in the formula (33); and
Ar₃₁₂, Ar₃₁₃, Ar₃₁₅, and Ar₃₁₆ each independently represent the same as Ar₃₁₂, Ar₃₁₃, Ar₃₁₅, and Ar₃₁₆ in the formula (33).

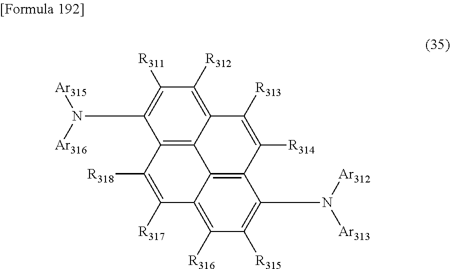

In the formula (35):
R₃₁₁ to R₃₁₈ each independently represent the same as R₃₀₁ to R₃₁₀ in the formula (3) that are not a monovalent group represented by the formula (31); and
Ar₃₁₂, Ar₃₁₃, Ar₃₁₅, and Ar₃₁₆ each independently represent the same as Ar₃₁₂, Ar₃₁₃, Ar₃₁₅, and Ar₃₁₆ in the formula (33).

In the formula (31), at least one of Ar₃₀₁ or Ar₃₀₂ is preferably a group represented by a formula (36).

In the formulae (33) to (35), at least one of Ar₃₁₂ or Ar₃₁₃ is preferably a group represented by the formula (36).

In the formulae (33) to (35), at least one of Ar₃₁₅ or Ar₃₁₆ is preferably a group represented by the formula (36).

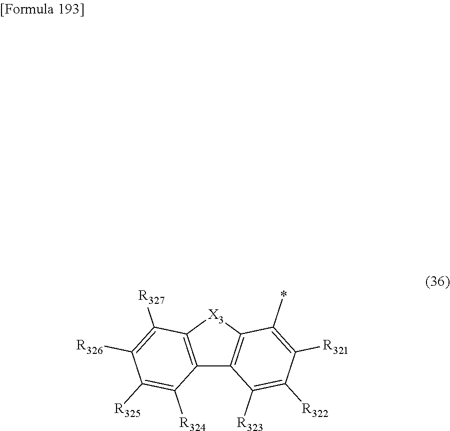

In the formula (36):
X₃ represents an oxygen atom or a sulfur atom;
at least one combination of adjacent two or more of R₃₂₁ to R₃₂₇ are mutually bonded to form a substituted or unsubstituted monocyclic ring, mutually bonded to form a substituted or unsubstituted fused ring, or not mutually bonded;
R₃₂₁ to R₃₂₇ not forming the monocyclic ring and not forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
* represents a bonding position with L₃₀₂, L₃₀₃, L₃₁₂, L₃₁₃, L₃₁₅, or L₃₁₆.

X₃ is preferably an oxygen atom.

At least one of R₃₂₁ to R₃₂₇ is preferably a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the formula (31), it is preferable that Ar₃₀₁ is a group represented by the formula (36) and Ar₃₀₂ is a substituted or unsubstituted aryl group having 6 to 50 ring carbon atoms.

In the formulae (33) to (35), it is preferable that Ar₃₁₂ is a group represented by the formula (36) and Ar₃₁₃ is a substituted or unsubstituted aryl group having 6 to 50 ring carbon atoms.

In the formulae (33) to (35), it is preferable that Ar₃₁₅ is a group represented by the formula (36) and Ar₃₁₆ is a substituted or unsubstituted aryl group having 6 to 50 ring carbon atoms.

In an exemplary embodiment, a compound represented by the formula (3) is represented by a formula (37) below.

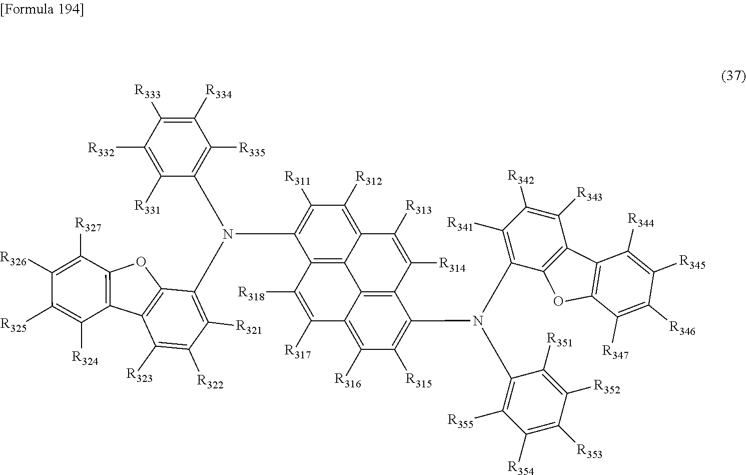

In the formula (37):
R₃₁₁ to R₃₁₈ each independently represent the same as R₃₀₁ to R₃₁₀ in the formula (3) that are not a monovalent group represented by the formula (31);
at least one combination of adjacent two or more of R₃₂₁ to R₃₂₇ are mutually bonded to form a substituted or unsubstituted monocyclic ring, mutually bonded to form a substituted or unsubstituted fused ring, or not mutually bonded;
at least one combination of adjacent two or more of R₃₄₁ to R₃₄₇ are mutually bonded to form a substituted or unsubstituted monocyclic ring, mutually bonded to form a substituted or unsubstituted fused ring, or not mutually bonded;
R₃₂₁ to R₃₂₇ and R₃₄₁ to R₃₄₇ not forming the monocyclic ring and not forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
R₃₃₁ to R₃₃₅ and R₃₅₁ to R₃₅₅ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

### Examples of Compound Represented by Formula (3)

Specific examples of a compound represented by the formula (3) are compounds shown below.

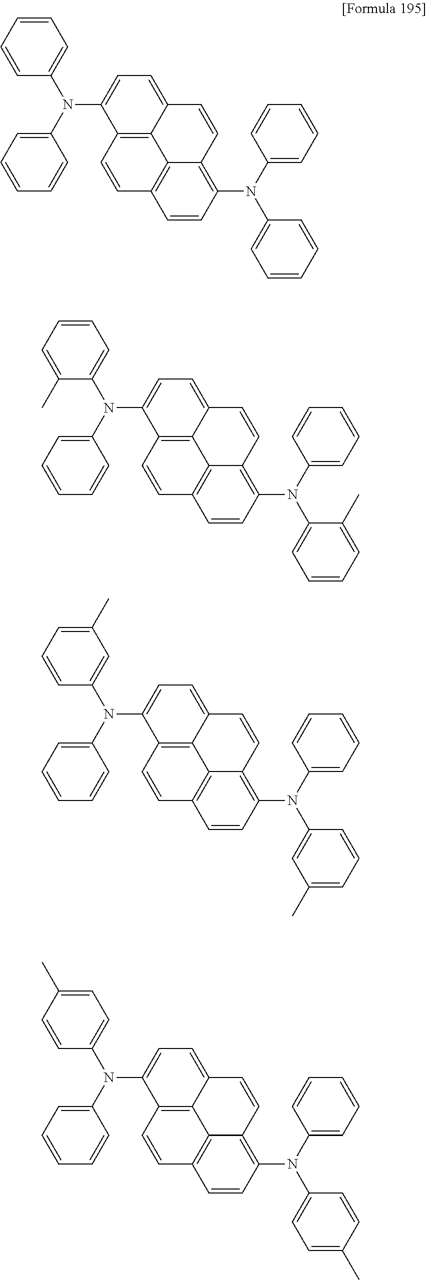 [SMILES: c1ccc(N(c2ccccc2)c2ccc3ccc4c(N(c5ccccc5)c5ccccc5)ccc5ccc2c3c54)cc1 ; Cc1ccccc1N(c1ccccc1)c1ccc2ccc3c(N(c4ccccc4)c4ccccc4C)ccc4ccc1c2c43 ; Cc1cccc(N(c2ccccc2)c2ccc3ccc4c(N(c5ccccc5)c5cccc(C)c5)ccc5ccc2c3c54)c1 ; Cc1ccc(N(c2ccccc2)c2ccc3ccc4c(N(c5ccccc5)c5ccc(C)cc5)ccc5ccc2c3c54)cc1]

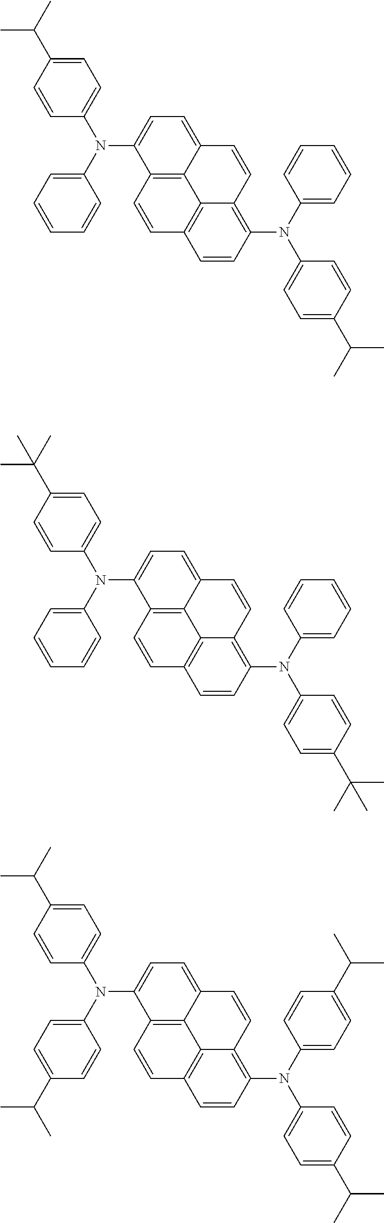

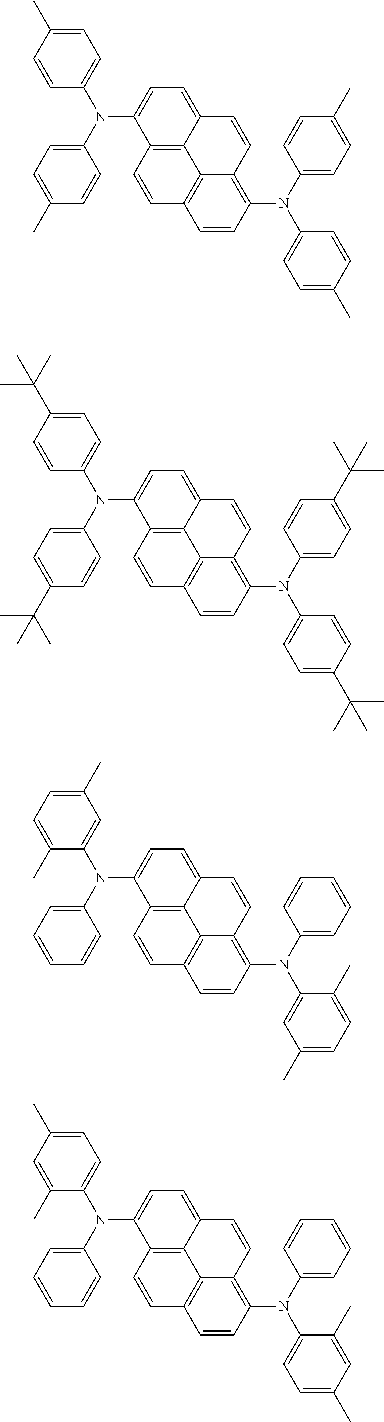

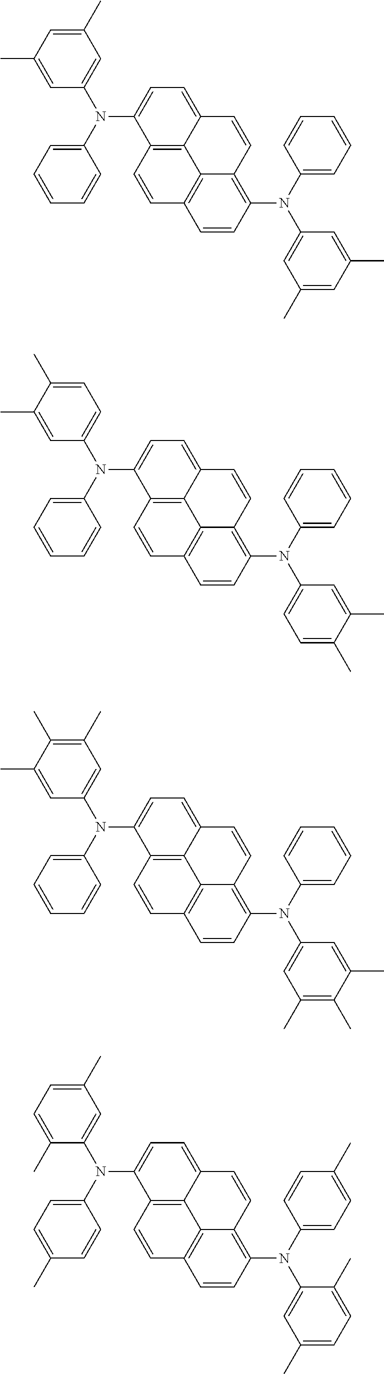

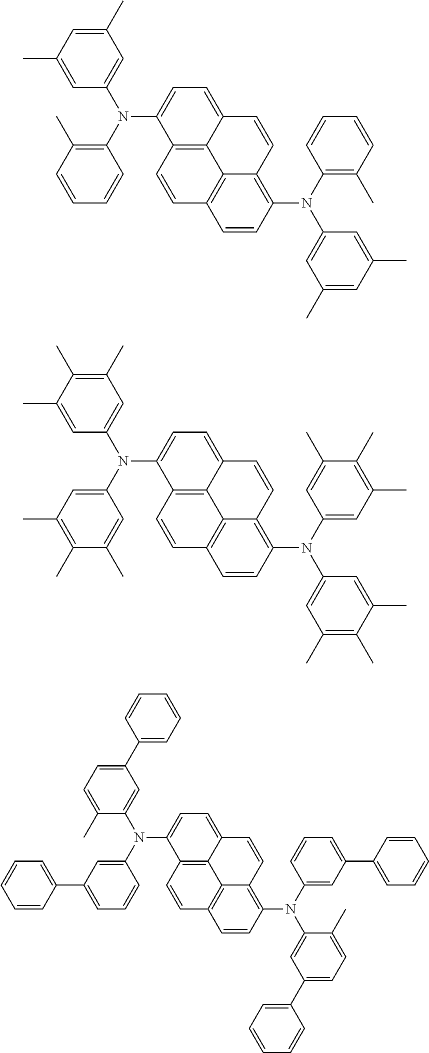

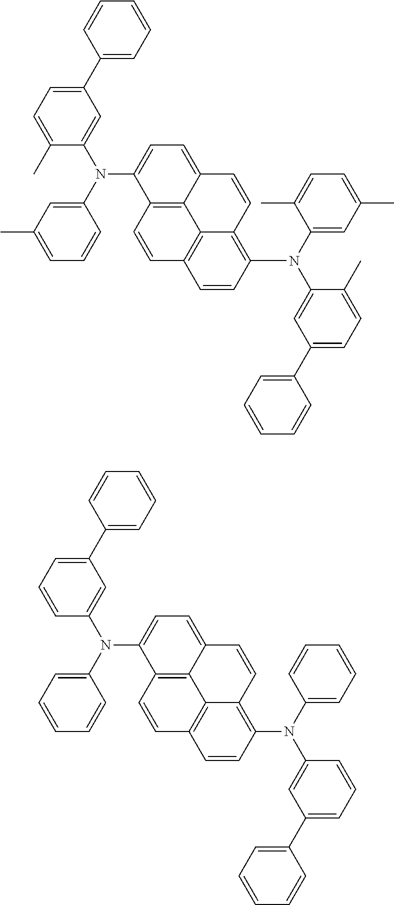

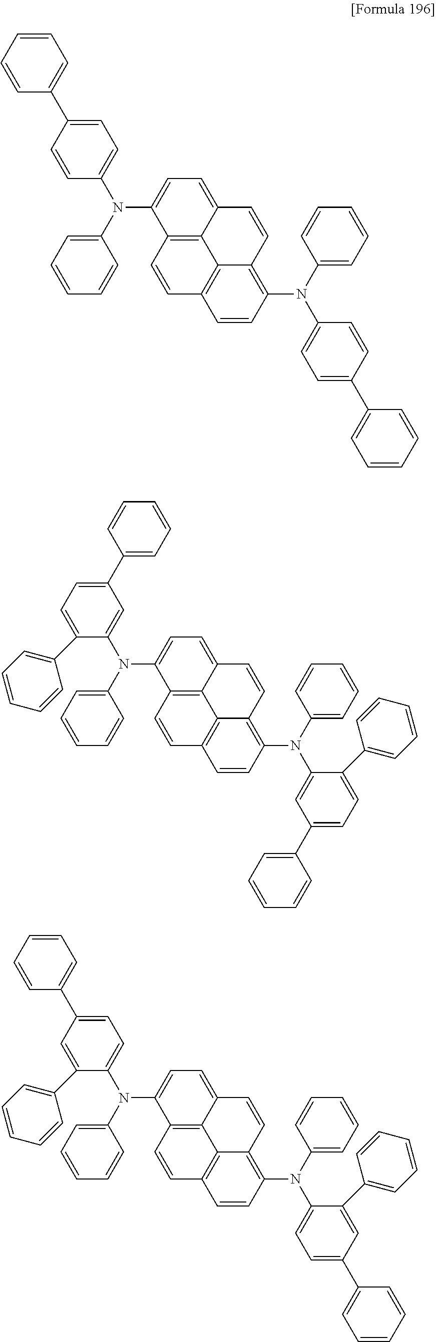 [SMILES: c1ccc(-c2ccc(N(c3ccccc3)c3ccc4ccc5c(N(c6ccccc6)c6ccc(-c7ccccc7)cc6)ccc6ccc3c4c65)cc2)cc1 ; c1ccc(-c2ccc(-c3ccccc3)c(N(c3ccccc3)c3ccc4ccc5c(N(c6ccccc6)c6cc(-c7ccccc7)ccc6-c6ccccc6)ccc6ccc3c4c65)c2)cc1 ; c1ccc(-c2ccc(N(c3ccccc3)c3ccc4ccc5c(N(c6ccccc6)c6ccc(-c7ccccc7)cc6-c6ccccc6)ccc6ccc3c4c65)c(-c3ccccc3)c2)cc1]

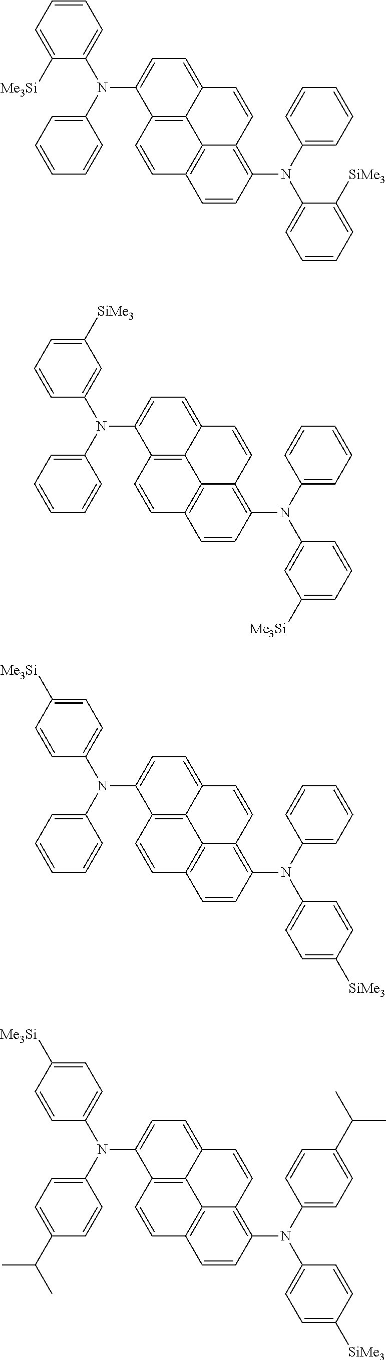 [SMILES: C[Si](C)(C)c1ccccc1N(c1ccccc1)c1ccc2ccc3c(N(c4ccccc4)c4ccccc4[Si](C)(C)C)ccc4ccc1c2c43 ; C[Si](C)(C)c1cccc(N(c2ccccc2)c2ccc3ccc4c(N(c5ccccc5)c5cccc([Si](C)(C)C)c5)ccc5ccc2c3c54)c1 ; C[Si](C)(C)c1ccc(N(c2ccccc2)c2ccc3ccc4c(N(c5ccccc5)c5ccc([Si](C)(C)C)cc5)ccc5ccc2c3c54)cc1 ; CC(C)c1ccc(N(c2ccc([Si](C)(C)C)cc2)c2ccc3ccc4c(N(c5ccc(C(C)C)cc5)c5ccc([Si](C)(C)C)cc5)ccc5ccc2c3c54)cc1]

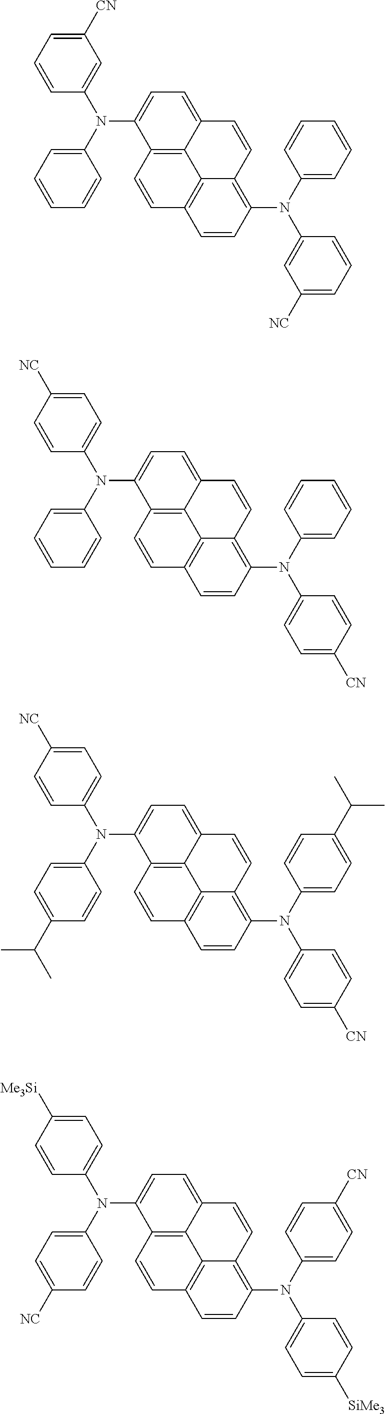 [SMILES: N#Cc1cccc(N(c2ccccc2)c2ccc3ccc4c(N(c5ccccc5)c5cccc(C#N)c5)ccc5ccc2c3c54)c1 ; N#Cc1ccc(N(c2ccccc2)c2ccc3ccc4c(N(c5ccccc5)c5ccc(C#N)cc5)ccc5ccc2c3c54)cc1 ; CC(C)c1ccc(N(c2ccc(C#N)cc2)c2ccc3ccc4c(N(c5ccc(C#N)cc5)c5ccc(C(C)C)cc5)ccc5ccc2c3c54)cc1 ; C[Si](C)(C)c1ccc(N(c2ccc(C#N)cc2)c2ccc3ccc4c(N(c5ccc(C#N)cc5)c5ccc([Si](C)(C)C)cc5)ccc5ccc2c3c54)cc1]

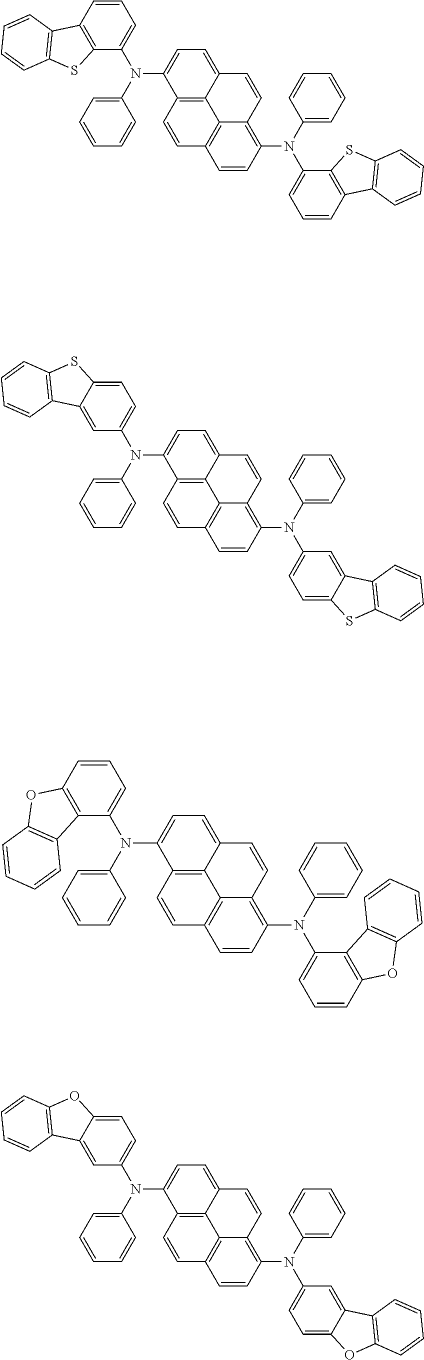 [SMILES: c1ccc(N(c2ccc3ccc4c(N(c5ccccc5)c5cccc6c5sc5ccccc56)ccc5ccc2c3c54)c2cccc3c2sc2ccccc23)cc1 ; c1ccc(N(c2ccc3sc4ccccc4c3c2)c2ccc3ccc4c(N(c5ccccc5)c5ccc6sc7ccccc7c6c5)ccc5ccc2c3c54)cc1 ; c1ccc(N(c2ccc3ccc4c(N(c5ccccc5)c5cccc6oc7ccccc7c56)ccc5ccc2c3c54)c2cccc3oc4ccccc4c23)cc1 ; c1ccc(N(c2ccc3oc4ccccc4c3c2)c2ccc3ccc4c(N(c5ccccc5)c5ccc6oc7ccccc7c6c5)ccc5ccc2c3c54)cc1]

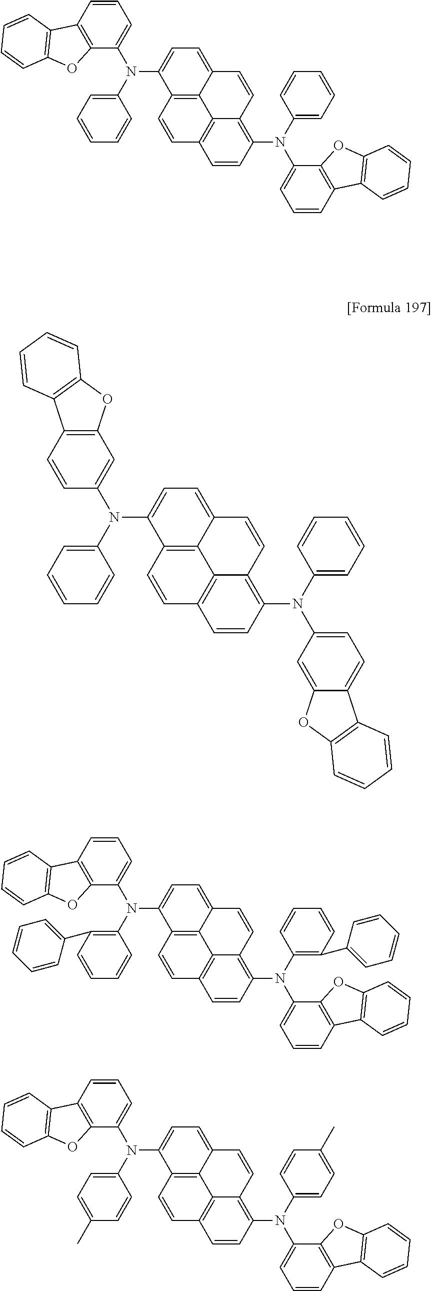 [SMILES: c1ccc(N(c2ccc3ccc4c(N(c5ccccc5)c5cccc6c5oc5ccccc56)ccc5ccc2c3c54)c2cccc3c2oc2ccccc23)cc1 ; c1ccc(N(c2ccc3c(c2)oc2ccccc23)c2ccc3ccc4c(N(c5ccccc5)c5ccc6c(c5)oc5ccccc56)ccc5ccc2c3c54)cc1 ; c1ccc(-c2ccccc2N(c2ccc3ccc4c(N(c5ccccc5-c5ccccc5)c5cccc6c5oc5ccccc56)ccc5ccc2c3c54)c2cccc3c2oc2ccccc23)cc1 ; Cc1ccc(N(c2ccc3ccc4c(N(c5ccc(C)cc5)c5cccc6c5oc5ccccc56)ccc5ccc2c3c54)c2cccc3c2oc2ccccc23)cc1]

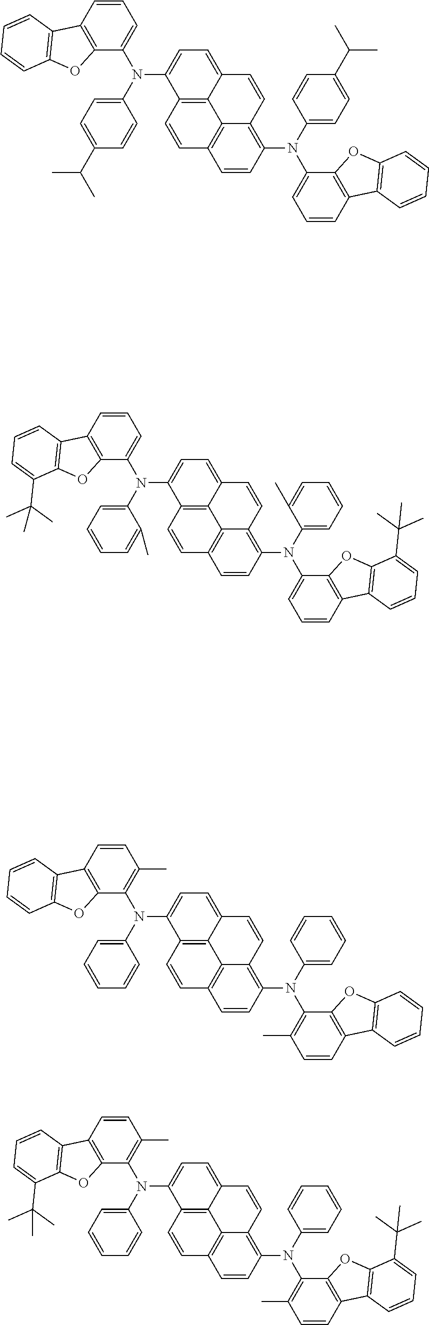

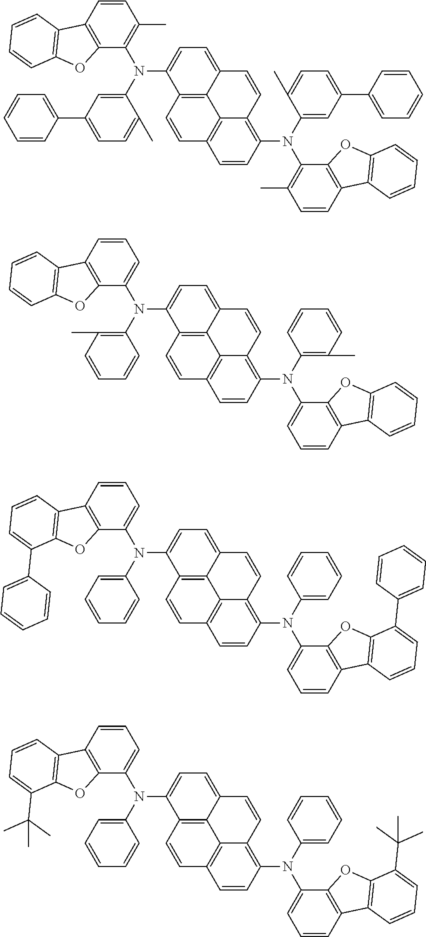 [SMILES: Cc1ccc(-c2ccccc2)cc1N(c1ccc2ccc3c(N(c4cc(-c5ccccc5)ccc4C)c4c(C)ccc5c4oc4ccccc45)ccc4ccc1c2c43)c1c(C)ccc2c1oc1ccccc12 ; Cc1ccccc1N(c1ccc2ccc3c(N(c4ccccc4C)c4cccc5c4oc4ccccc45)ccc4ccc1c2c43)c1cccc2c1oc1ccccc12 ; c1ccc(-c2cccc3c2oc2c(N(c4ccccc4)c4ccc5ccc6c(N(c7ccccc7)c7cccc8c7oc7c(-c9ccccc9)cccc78)ccc7ccc4c5c76)cccc23)cc1 ; CC(C)(C)c1cccc2c1oc1c(N(c3ccccc3)c3ccc4ccc5c(N(c6ccccc6)c6cccc7c6oc6c(C(C)(C)C)cccc67)ccc6ccc3c4c65)cccc12]

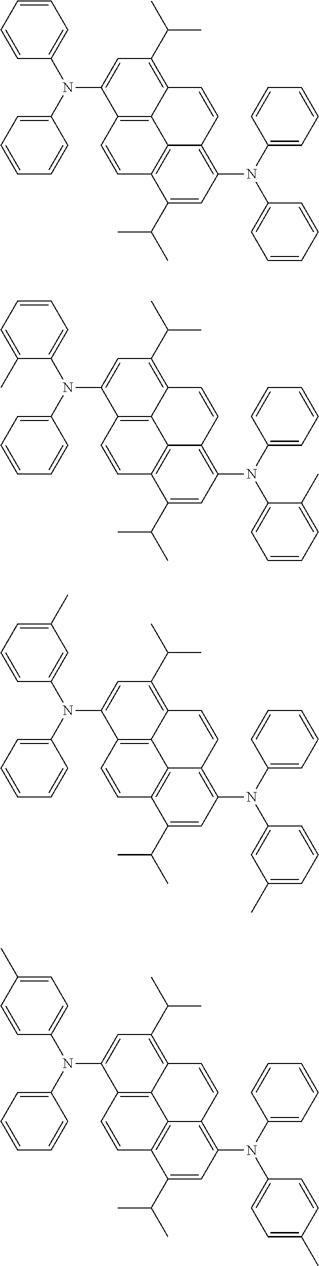

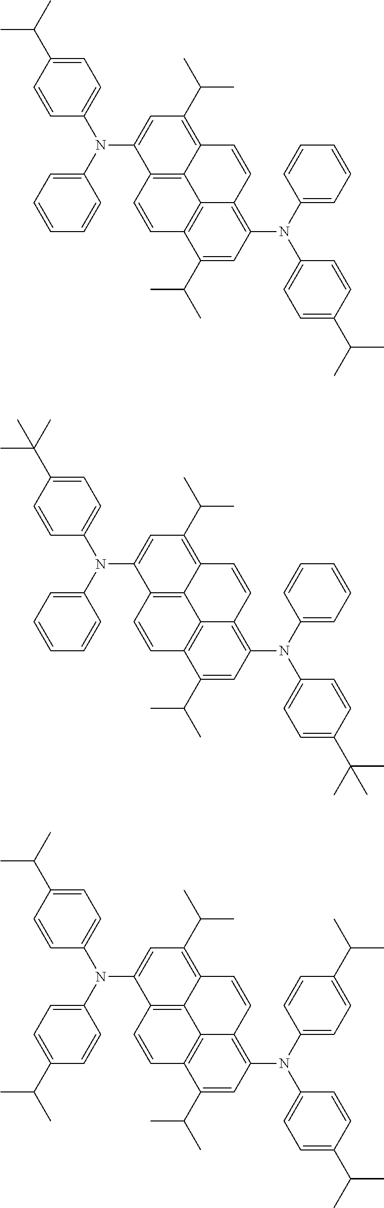

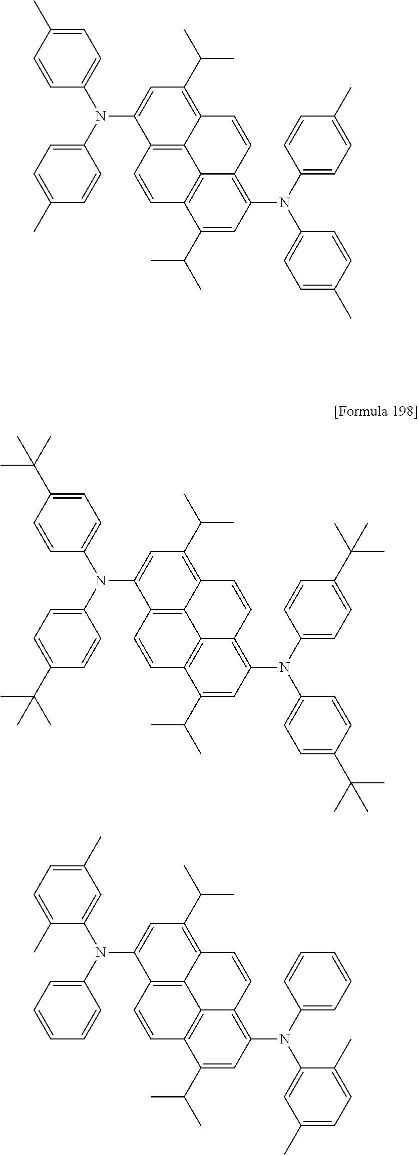

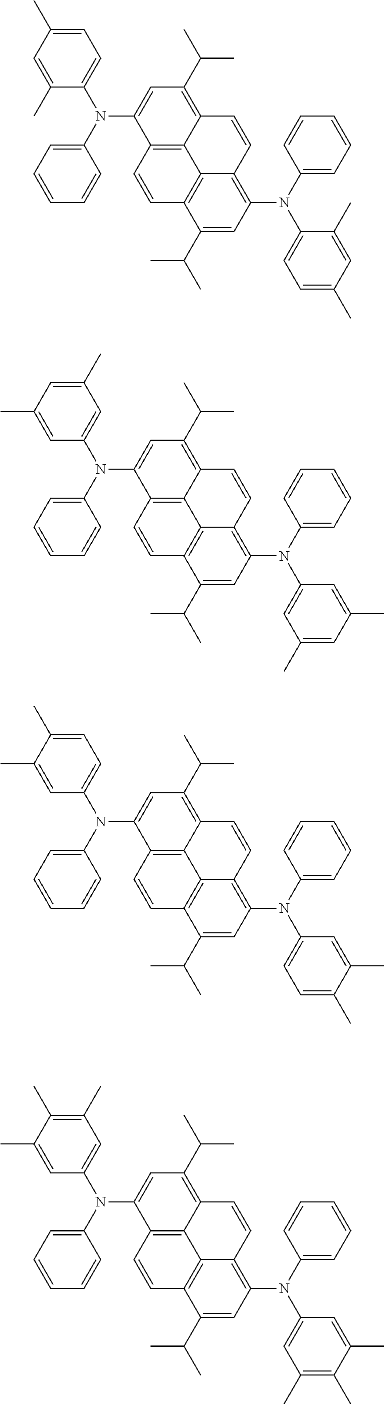

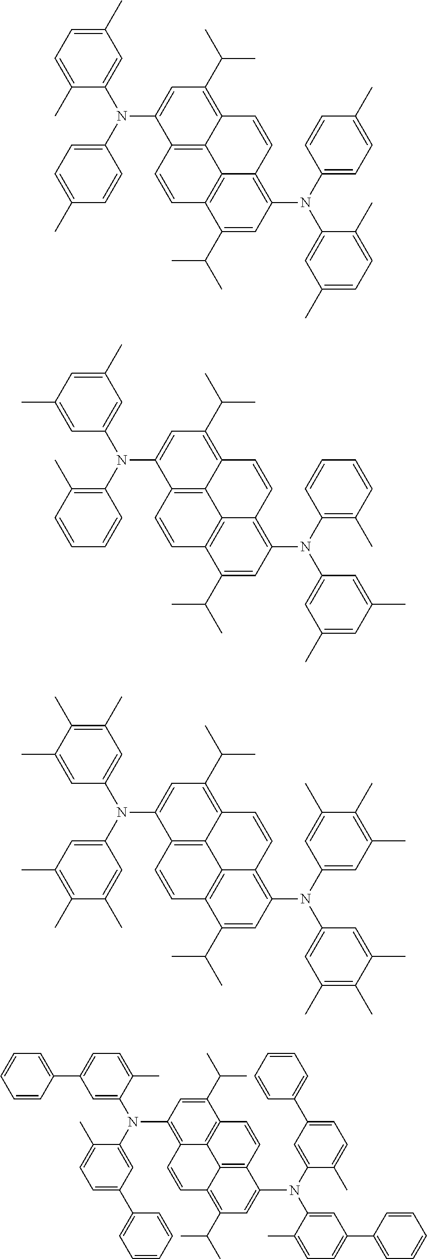

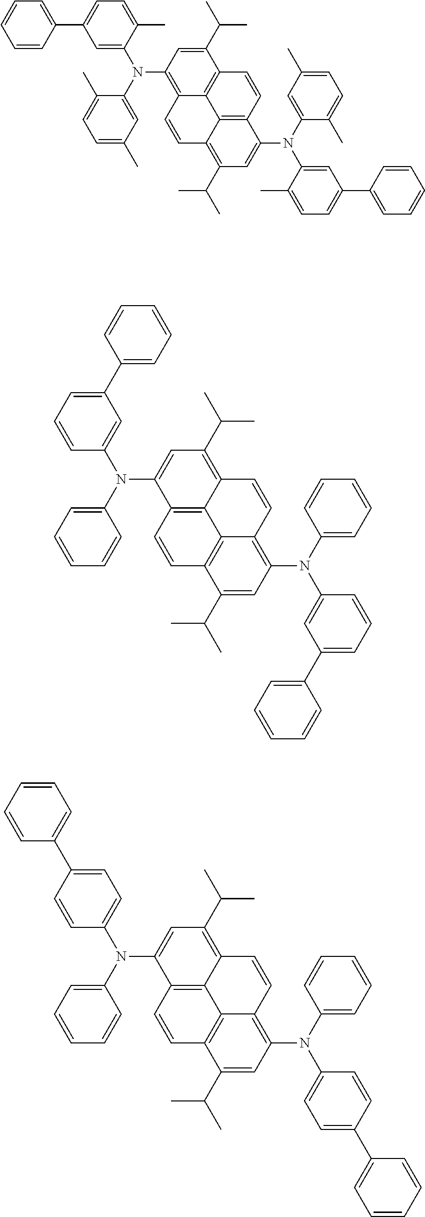 [SMILES: Cc1ccc(C)c(N(c2cc(-c3ccccc3)ccc2C)c2cc(C(C)C)c3ccc4c(N(c5cc(C)ccc5C)c5cc(-c6ccccc6)ccc5C)cc(C(C)C)c5ccc2c3c54)c1 ; CC(C)c1cc(N(c2ccccc2)c2cccc(-c3ccccc3)c2)c2ccc3c(C(C)C)cc(N(c4ccccc4)c4cccc(-c5ccccc5)c4)c4ccc1c2c34 ; CC(C)c1cc(N(c2ccccc2)c2ccc(-c3ccccc3)cc2)c2ccc3c(C(C)C)cc(N(c4ccccc4)c4ccc(-c5ccccc5)cc4)c4ccc1c2c34]

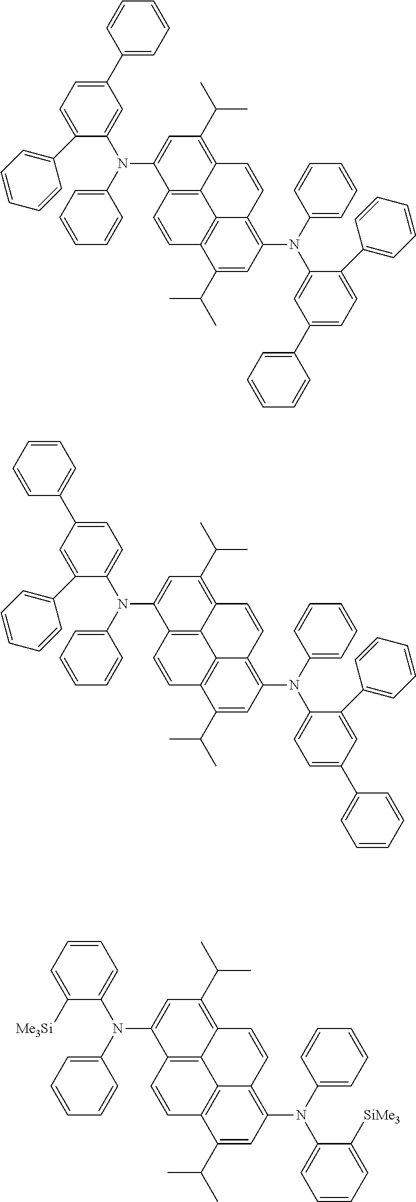 [SMILES: CC(C)c1cc(N(c2ccccc2)c2cc(-c3ccccc3)ccc2-c2ccccc2)c2ccc3c(C(C)C)cc(N(c4ccccc4)c4cc(-c5ccccc5)ccc4-c4ccccc4)c4ccc1c2c34 ; CC(C)c1cc(N(c2ccccc2)c2ccc(-c3ccccc3)cc2-c2ccccc2)c2ccc3c(C(C)C)cc(N(c4ccccc4)c4ccc(-c5ccccc5)cc4-c4ccccc4)c4ccc1c2c34 ; CC(C)c1cc(N(c2ccccc2)c2ccccc2[Si](C)(C)C)c2ccc3c(C(C)C)cc(N(c4ccccc4)c4ccccc4[Si](C)(C)C)c4ccc1c2c34]

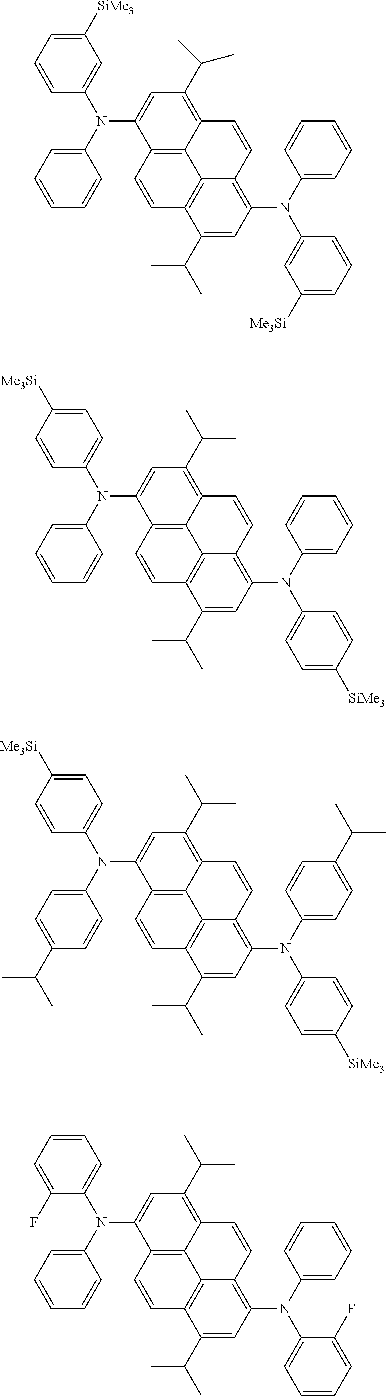 [SMILES: CC(C)c1cc(N(c2ccccc2)c2cccc([Si](C)(C)C)c2)c2ccc3c(C(C)C)cc(N(c4ccccc4)c4cccc([Si](C)(C)C)c4)c4ccc1c2c34 ; CC(C)c1cc(N(c2ccccc2)c2ccc([Si](C)(C)C)cc2)c2ccc3c(C(C)C)cc(N(c4ccccc4)c4ccc([Si](C)(C)C)cc4)c4ccc1c2c34 ; CC(C)c1ccc(N(c2ccc([Si](C)(C)C)cc2)c2cc(C(C)C)c3ccc4c(N(c5ccc(C(C)C)cc5)c5ccc([Si](C)(C)C)cc5)cc(C(C)C)c5ccc2c3c54)cc1 ; CC(C)c1cc(N(c2ccccc2)c2ccccc2F)c2ccc3c(C(C)C)cc(N(c4ccccc4)c4ccccc4F)c4ccc1c2c34]

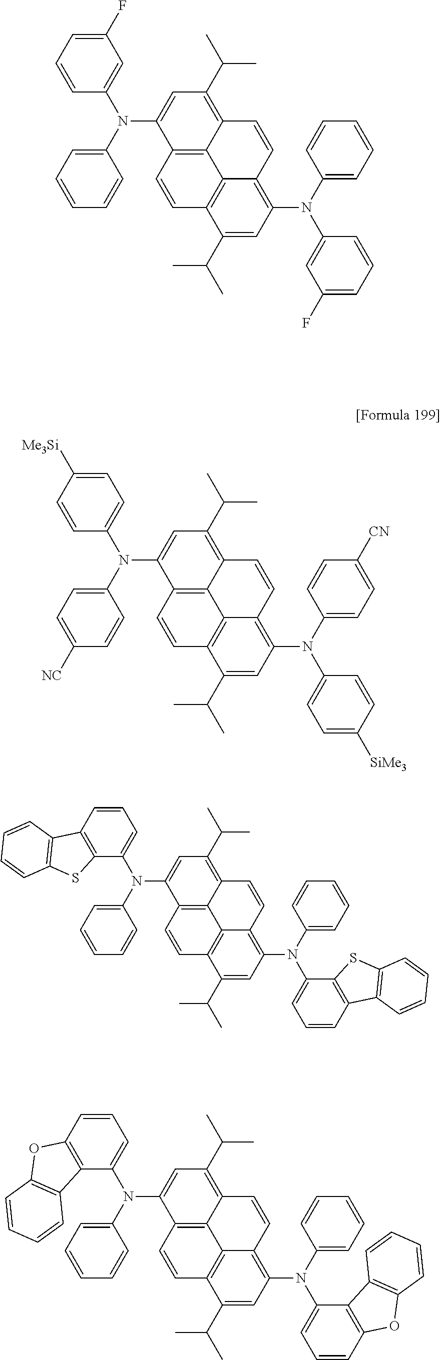 [SMILES: CC(C)c1cc(N(c2ccccc2)c2cccc(F)c2)c2ccc3c(C(C)C)cc(N(c4ccccc4)c4cccc(F)c4)c4ccc1c2c34 ; CC(C)c1cc(N(c2ccc(C#N)cc2)c2ccc([Si](C)(C)C)cc2)c2ccc3c(C(C)C)cc(N(c4ccc(C#N)cc4)c4ccc([Si](C)(C)C)cc4)c4ccc1c2c34 ; CC(C)c1cc(N(c2ccccc2)c2cccc3c2sc2ccccc23)c2ccc3c(C(C)C)cc(N(c4ccccc4)c4cccc5c4sc4ccccc45)c4ccc1c2c34 ; CC(C)c1cc(N(c2ccccc2)c2cccc3oc4ccccc4c23)c2ccc3c(C(C)C)cc(N(c4ccccc4)c4cccc5oc6ccccc6c45)c4ccc1c2c34]

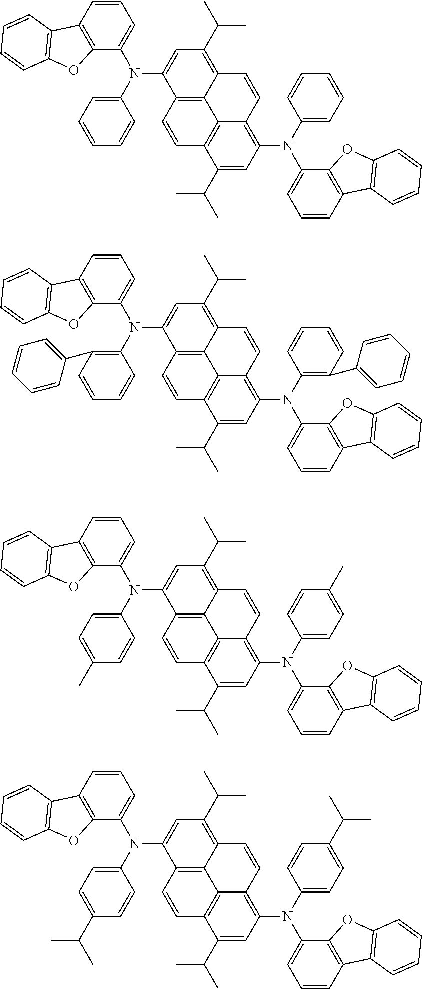 [SMILES: CC(C)c1cc(N(c2ccccc2)c2cccc3c2oc2ccccc23)c2ccc3c(C(C)C)cc(N(c4ccccc4)c4cccc5c4oc4ccccc45)c4ccc1c2c34 ; CC(C)c1cc(N(c2ccccc2-c2ccccc2)c2cccc3c2oc2ccccc23)c2ccc3c(C(C)C)cc(N(c4ccccc4-c4ccccc4)c4cccc5c4oc4ccccc45)c4ccc1c2c34 ; Cc1ccc(N(c2cc(C(C)C)c3ccc4c(N(c5ccc(C)cc5)c5cccc6c5oc5ccccc56)cc(C(C)C)c5ccc2c3c54)c2cccc3c2oc2ccccc23)cc1 ; CC(C)c1ccc(N(c2cc(C(C)C)c3ccc4c(N(c5ccc(C(C)C)cc5)c5cccc6c5oc5ccccc56)cc(C(C)C)c5ccc2c3c54)c2cccc3c2oc2ccccc23)cc1]

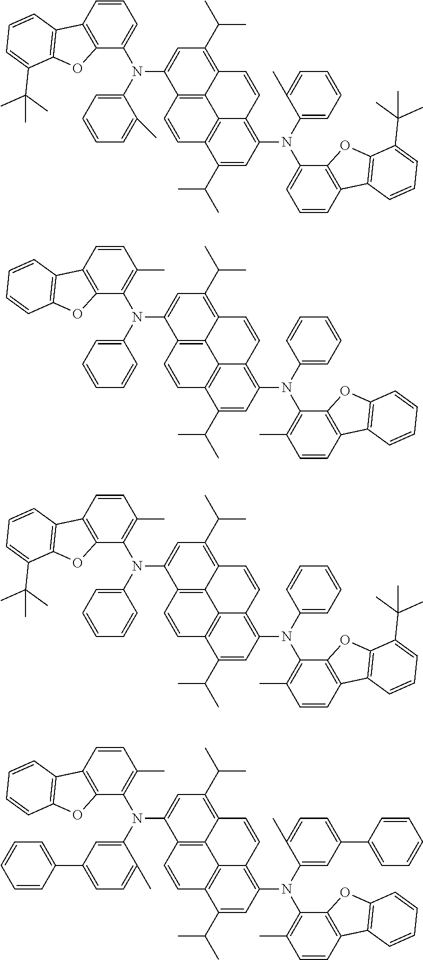

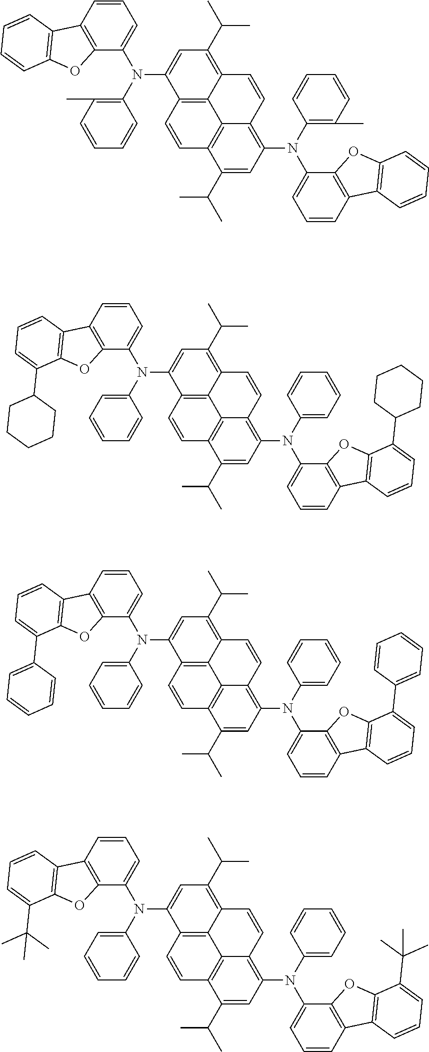 [SMILES: Cc1ccccc1N(c1cc(C(C)C)c2ccc3c(N(c4ccccc4C)c4cccc5c4oc4ccccc45)cc(C(C)C)c4ccc1c2c43)c1cccc2c1oc1ccccc12 ; CC(C)c1cc(N(c2ccccc2)c2cccc3c2oc2c(C4CCCCC4)cccc23)c2ccc3c(C(C)C)cc(N(c4ccccc4)c4cccc5c4oc4c(C6CCCCC6)cccc45)c4ccc1c2c34 ; CC(C)c1cc(N(c2ccccc2)c2cccc3c2oc2c(-c4ccccc4)cccc23)c2ccc3c(C(C)C)cc(N(c4ccccc4)c4cccc5c4oc4c(-c6ccccc6)cccc45)c4ccc1c2c34 ; CC(C)c1cc(N(c2ccccc2)c2cccc3c2oc2c(C(C)(C)C)cccc23)c2ccc3c(C(C)C)cc(N(c4ccccc4)c4cccc5c4oc4c(C(C)(C)C)cccc45)c4ccc1c2c34]

### Compound Represented by Formula (4)

The compound represented by the formula (4) will be described below.

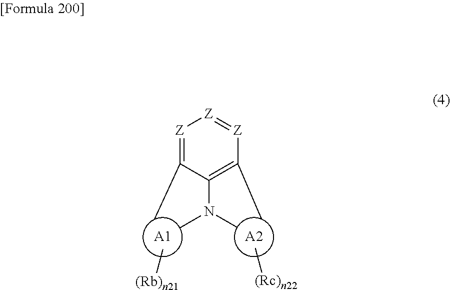

In the formula (4): Z are each independently CRa or a nitrogen atom;
A1 ring and A2 ring are each independently a substituted or unsubstituted aromatic hydrocarbon ring having 6 to 50 ring carbon atoms or a substituted or unsubstituted heterocycle having 5 to 50 ring atoms;
when a plurality of Ra are present, at least one combination of adjacent two or more of Ra are mutually bonded to form a substituted or unsubstituted monocyclic ring, mutually bonded to form a substituted or unsubstituted fused ring, or not mutually bonded;
n21 and n22 are each independently 0, 1, 2, 3 or 4;
when a plurality of Rb are present, at least one combination of adjacent two or more of Rb are mutually bonded to form a substituted or unsubstituted monocyclic ring, mutually bonded to form a substituted or unsubstituted fused ring, or not mutually bonded;
when a plurality of Rc are present, at least one combination of adjacent two or more of Rc are mutually bonded to form a substituted or unsubstituted monocyclic ring, mutually bonded to form a substituted or unsubstituted fused ring, or not mutually bonded; and
Ra, Rb, and Rc not forming the monocyclic ring and not forming the fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

The “aromatic hydrocarbon ring” for the A1 ring and A2 ring has the same structure as the compound formed by introducing a hydrogen atom to the “aryl group” described above.

Ring atoms of the “aromatic hydrocarbon ring” for the A1 ring and the A2 ring include two carbon atoms on a fused bicyclic structure at the center of the formula (4).

Specific examples of the “substituted or unsubstituted aromatic hydrocarbon ring having 6 to 50 ring carbon atoms” include a compound formed by introducing a hydrogen atom to the “aryl group” described in the specific example group G1.

The “heterocycle” for the A1 ring and A2 ring has the same structure as the compound formed by introducing a hydrogen atom to the “heterocyclic group” described above.

Ring atoms of the “heterocycle” for the A1 ring and the A2 ring include two carbon atoms on a fused bicyclic structure at the center of the formula (4).

Specific examples of the “substituted or unsubstituted heterocycle having 5 to 50 ring atoms” include a compound formed by introducing a hydrogen atom to the “heterocyclic group” described in the specific example group G2.

Rb is bonded to any one of carbon atoms forming the aromatic hydrocarbon ring for the A1 ring or any one of the atoms forming the heterocycle for the A1 ring.

Rc is bonded to any one of carbon atoms forming the aromatic hydrocarbon ring for the A2 ring or any one of the atoms forming the heterocycle for the A2 ring.

At least one of Ra, Rb, or Rc is preferably a group represented by the formula (4a) below. More preferably, at least two of Ra, Rb, and Rc are groups represented by the formula (4a).

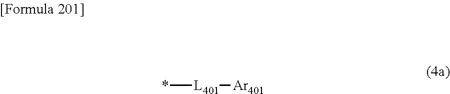

In the formula (4a): L₄₀₁ is preferably a single bond, a substituted or unsubstituted arylene group having 6 to 30 ring carbon atoms, or a substituted or unsubstituted divalent heterocyclic group having 5 to 30 ring atoms; and
Ar₄₀₁ is a substituted or unsubstituted aryl group having 6 to 50 ring carbon atoms, a substituted or unsubstituted heterocyclic group having 5 to 50 ring atoms, or a group represented by a formula (4b) below.

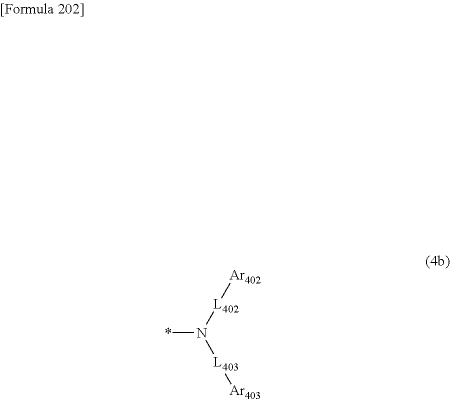

In the formula (4b): L₄₀₂ and L₄₀₃ are each independently a single bond, a substituted or unsubstituted arylene group having 6 to 30 ring carbon atoms, or a substituted or unsubstituted divalent heterocyclic group having 5 to 30 ring atoms;
a combination of Ar₄₀₂ and Ar₄₀₃ are mutually bonded to form a substituted or unsubstituted monocyclic ring, mutually bonded to form a substituted or unsubstituted fused ring, or not mutually bonded; and
Ar₄₀₂ and Ar₄₀₃ not forming the monocyclic ring and not forming the fused ring are each independently a substituted or unsubstituted aryl group having 6 to 50 ring carbon atoms, or a substituted or unsubstituted heterocyclic group having 5 to 50 ring atoms.

In an exemplary embodiment, the compound represented by the formula (4) is represented by a formula (42) below.

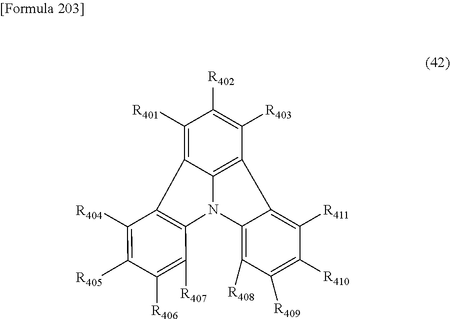

In the formula (42): at least one combination of adjacent two or more of R₄₀₁ to R₄₁₁ are mutually bonded to form a substituted or unsubstituted monocyclic ring, mutually bonded to form a substituted or unsubstituted fused ring, or not mutually bonded; and
R₄₀₁ to R₄₁₁ neither forming the monocyclic ring nor forming the fused ring each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

At least one of R₄₀₁ to R₄₁₁ is preferably a group represented by the formula (4a). More preferably, at least two of R₄₀₁ to R₄₁₁ are groups represented by the formula (21a).

R₄₀₄ and R₄₁₁ are preferably groups represented by the formula (4a).

In an exemplary embodiment, the compound represented by the formula (4) is a compound formed by bonding a moiety represented by a formula (4-1) or a formula (4-2) below to the A1 ring.

Further, in an exemplary embodiment, the compound represented by the formula (42) is a compound formed by bonding the moiety represented by the formula (4-1) or the formula (4-2) to the ring bonded with R₄₀₄ to R₄₀₇.

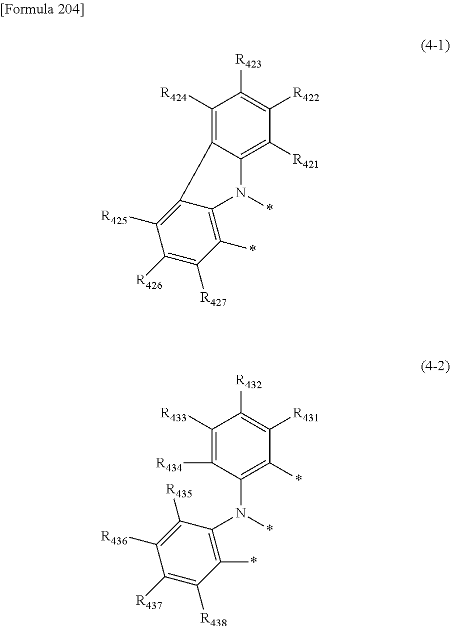

In the formula (4-1), two * are each independently bonded to the ring carbon atom of the aromatic hydrocarbon ring or the ring atom of the heterocycle for the A1 ring in the formula (4) or bonded to one of R₄₀₄ to R₄₀₇ in the formula (42).

In the formula (4-2), three * are each independently bonded to the ring carbon atom of the aromatic hydrocarbon ring or the ring atom of the heterocycle for the A1 ring in the formula (4) or bonded to one of R₄₀₄ to R₄₀₇ in the formula (42).

At least one combination of adjacent two or more of R₄₂₁ to R₄₂₇ are mutually bonded to form a substituted or unsubstituted monocyclic ring, mutually bonded to form a substituted or unsubstituted fused ring, or not mutually bonded.

At least one combination of adjacent two or more of R₄₃₁ to R₄₃₈ are mutually bonded to form a substituted or unsubstituted monocyclic ring, mutually bonded to form a substituted or unsubstituted fused ring, or not mutually bonded.

R₄₂₁ to R₄₂₇ and R₄₃₁ to R₄₃₈ neither forming the monocyclic ring nor forming the fused ring each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, the compound represented by the formula (4) is a compound represented by a formula (41-3), a formula (41-4) or a formula (41-5) below.

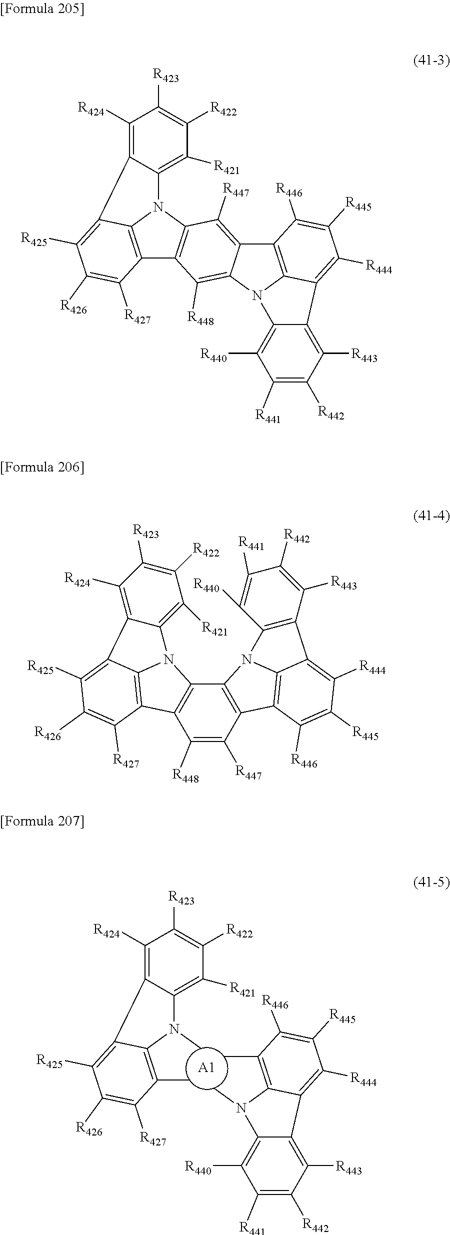

In the formulae (41-3), (41-4), and (41-5):
A1 ring is as defined for the formula (4);
R₄₂₁ to R₄₂₇ each independently represent the same as R₄₂₁ to R₄₂₇ in the formula (4-1); and
R₄₄₀ to R₄₄₈ each independently represent the same as R₄₀₁ to R₄₁₁ in the formula (42).

In an exemplary embodiment, a substituted or unsubstituted aromatic hydrocarbon ring having 6 to 50 ring carbon atoms for the A1 ring in the formula (41-5) is a substituted or unsubstituted naphthalene ring, or a substituted or unsubstituted fluorene ring.

In an exemplary embodiment, a substituted or unsubstituted heterocycle having 5 to 50 ring atoms for the A1 ring in the formula (41-5) is a substituted or unsubstituted dibenzofuran ring, a substituted or unsubstituted carbazole ring, or a substituted or unsubstituted dibenzothiophene ring.

In an exemplary embodiment, the compound represented by the formula (4) or the formula (42) is a compound selected from the group consisting of compounds represented by formulae (461) to (467) below.

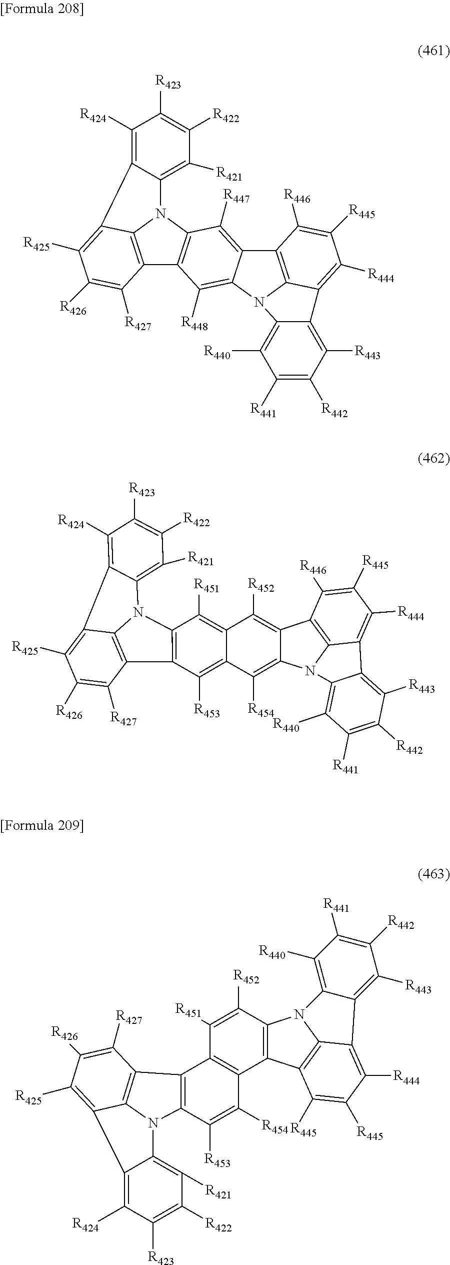

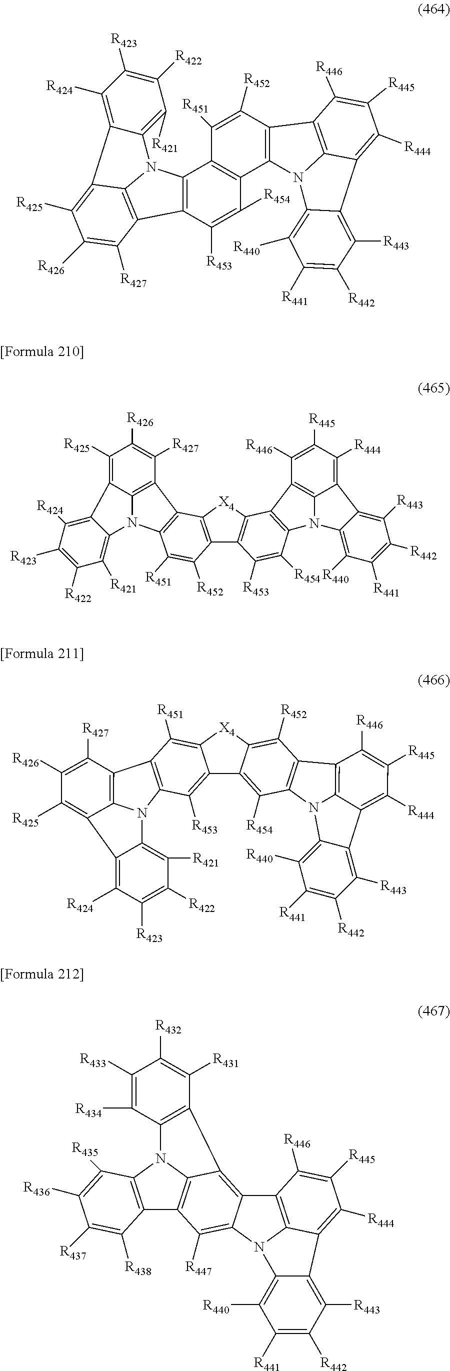

In the formulae (461), (462), (463), (464), (465), (466), and (467):
R₄₂₁ to R₄₂₇ each independently represent the same as R₄₂₁ to R₄₂₇ in the formula (4-1);
R₄₃₁ to R₄₃₈ each independently represent the same as R₄₃₁ to R₄₃₈ in the formula (4-2);
R₄₄₀ to R₄₄₈ and R₄₅₁ to R₄₅₄ each independently represent the same as R₄₀₁ to R₄₁₁ in the formula (42);
X₄ is an oxygen atom, NR₈₀₁, or C(R₈₀₂)(R₈₀₃);
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

In an exemplary embodiment, in the compound represented by the formula (42), at least one combination of adjacent two or more of R₄₀₁ to R₄₁₁ are mutually bonded to form a substituted or unsubstituted monocyclic ring or a substituted or unsubstituted fused ring. The compound represented by the formula (42) in the exemplary embodiment is described in detail as a compound represented by a formula (45).

### Compound Represented by Formula (45)

The compound represented by the formula (45) will be described.

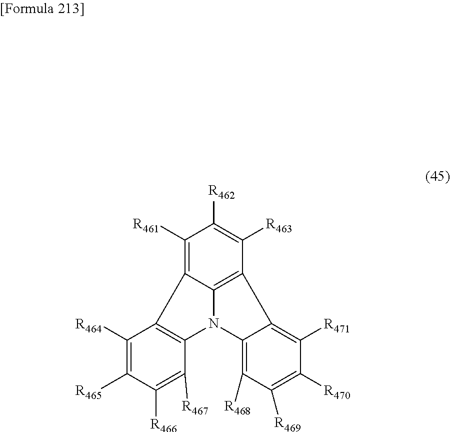

In the formula (45), two or more of combinations selected from the group consisting of a combination of R₄₆₁ and R₄₆₂, a combination of R₄₆₂ and R₄₆₃, a combination of R₄₆₄ and R₄₆₅, a combination of R₄₆₅ and R₄₆₆, a combination of R₄₆₆ and R₄₆₇, a combination of R₄₆₈ and R₄₆₉, a combination of R₄₆₉ and R₄₇₀, and a combination of R₄₇₀ and R₄₇₁ are mutually bonded to form a substituted or unsubstituted monocyclic ring or a substituted or unsubstituted fused ring.

However, the combination of R₄₆₁ and R₄₆₂ and the combination of R₄₆₂ and R₄₆₃, the combination of R₄₆₄ and R₄₆₅ and the combination of R₄₆₅ and R₄₆₆, the combination of R₄₆₅ and R₄₆₆ and the combination of R₄₆₆ and R₄₆₇, the combination of R₄₆₈ and R₄₆₉ and the combination of R₄₆₉ and R₄₇₀, and the combination of R₄₆₉ and R₄₇₀ and the combination of R₄₇₀ and R₄₇₁ do not form a ring at the same time.

At least two rings formed by R₄₆₁ to R₄₇₁ are mutually the same or different.

R₄₆₁ to R₄₇₁ neither forming the monocyclic ring nor forming the fused ring each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (45), Rₙ and Rₙ₊₁ (n being an integer selected from 461, 462, 464 to 466, and 468 to 470) are mutually bonded to form a substituted or unsubstituted monocyclic ring or fused ring together with two ring carbon atoms bonded with Rₙ and Rₙ₊₁. The ring is preferably formed of atoms selected from the group consisting of a carbon atom, an oxygen atom, a sulfur atom, and a nitrogen atom, and is made of 3 to 7, more preferably 5 or 6 atoms.

The number of the above cyclic structures in the compound represented by the formula (45) is, for instance, 2, 3, or 4. The two or more of the cyclic structures may be present on the same benzene ring on the basic skeleton represented by the formula (45) or may be present on different benzene rings. For instance, when three cyclic structures are present, each of the cyclic structures may be present on corresponding one of the three benzene rings of the formula (45).

Examples of the above cyclic structures in the compound represented by the formula (45) include structures represented by formulae (451) to (460) below.

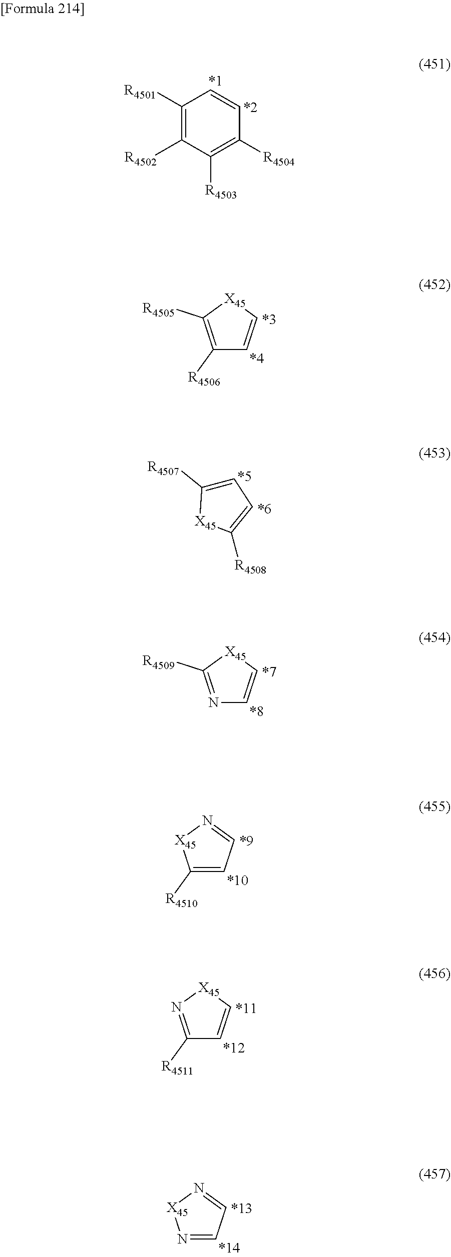

In the formulae (451) to (457):
each combination of *1 and *2, *3 and *4, *5 and *6, *7 and *8, *9 and *10, *11 and *12, and *13 and *14 represent the two ring carbon atoms respectively bonded with Rₙ and Rₙ₊₁;
the ring carbon atom bonded with Rₙ may be any one of the two ring carbon atoms represented by *1 and *2, *3 and *4, *5 and *6, *7 and *8, *9 and *10, *11 and *12, and *13 and *14;
X₄₅ is C(R₄₅₁₂)(R₄₅₁₃), NR₄₅₁₄, an oxygen atom, or a sulfur atom;
at least one combination of adjacent two or more of R₄₅₀₁ to R₄₅₀₆ and R₄₅₁₂ to R₄₅₁₃ are mutually bonded to form a substituted or unsubstituted monocyclic ring, mutually bonded to form a substituted or unsubstituted fused ring, or not mutually bonded; and
R₄₅₀₁ to R₄₅₁₄ not forming the monocyclic ring and not forming the fused ring represent the same as R₄₆₁ to R₄₇₁ in the formula (45).

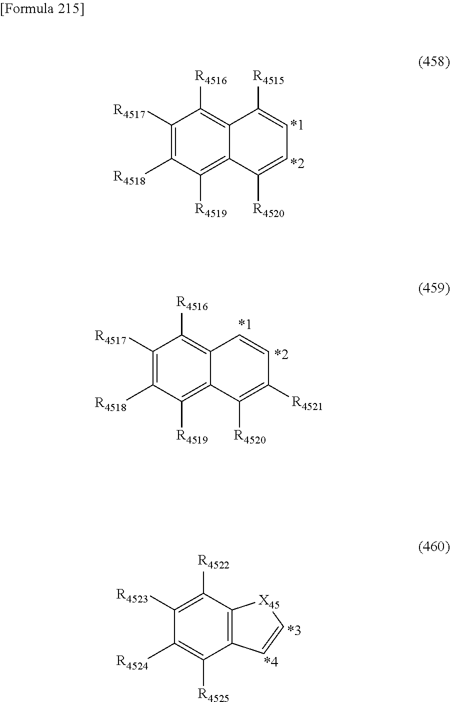

In the formulae (458) to (460):
each combination of *1 and *2, and *3 and *4 represent the two ring carbon atoms each bonded with Rₙ and Rₙ₊₁;
the ring carbon atom bonded with Rₙ may be any one of the two ring carbon atoms represented by *1 and *2, or *3 and *4;
X₄₅ is C(R₄₅₁₂)(R₄₅₁₃), NR₄₅₁₄, an oxygen atom, or a sulfur atom;
at least one combination of adjacent two or more of R₄₅₁₂, R₄₅₁₃ and R₄₅₁₅ to R₄₅₂₅ are mutually bonded to form a substituted or unsubstituted monocyclic ring, mutually bonded to form a substituted or unsubstituted fused ring, or not mutually bonded; and
R₄₅₁₂, R₄₅₁₃, R₄₅₁₅ to R₄₅₂₁ and R₄₅₂₂ to R₄₅₂₅ not forming the monocyclic ring and not forming the fused ring, and R₄₅₁₄ represent the same as R₄₆₁ to R₄₇₁ in the formula (45).

In the formula (45), it is preferable that at least one of R₄₆₂, R₄₆₄, R₄₆₅, R₄₇₀ or R₄₇₁ (preferably, at least one of R₄₆₂, R₄₆₅ or R₄₇₀, more preferably R₄₆₂) is a group not forming the cyclic structure.
(i) A substituent, if present, of the cyclic structure formed by Rₙ and Rₙ₊₁ of the formula (45),
(ii) R₄₆₁ to R₄₇₁ not forming the cyclic structure in the formula (45), and
(iii) R₄₅₀₁ to R₄₅₁₄ and R₄₅₁₅ to R₄₅₂₅ in the formulae (451) to (460) are each independently preferably selected from the group consisting of a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), a substituted or unsubstituted aryl group having 6 to 50 ring carbon atoms, a substituted or unsubstituted heterocyclic group having 5 to 50 ring atoms, or groups represented by formulae (461) to (464).

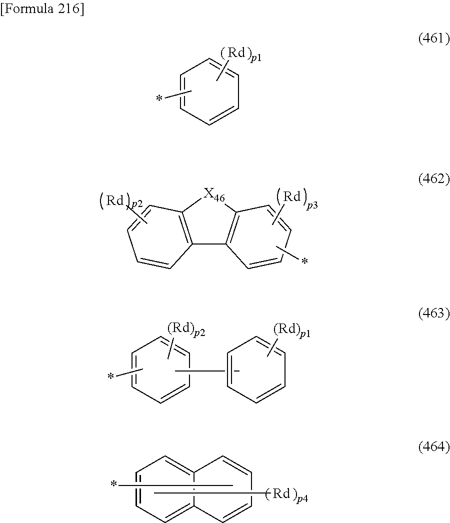

In the formulae (461) to (464): R_{d} each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
X₄₆ represents C(R₈₀₁)(R₈₀₂), NR₈₀₃, an oxygen atom or a sulfur atom;
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different; and
when a plurality of R₈₀₂ are present, the plurality of R₈₀₂ are mutually the same or different;
when a plurality of R₈₀₃ are present, the plurality of R₈₀₃ are mutually the same or different;
p1 is 5;
p2 is 4;
p3 is 3;
p4 is 7; and
* in the formulae (461) to (464) each independently represents a bonding position with a ring structure.

In the third and fourth compounds, R₉₀₁ to R₉₀₇ represent the same as those as described above.

In an exemplary embodiment, the compound represented by the formula (45) is represented by one of formulae (45-1) to (45-6) below.

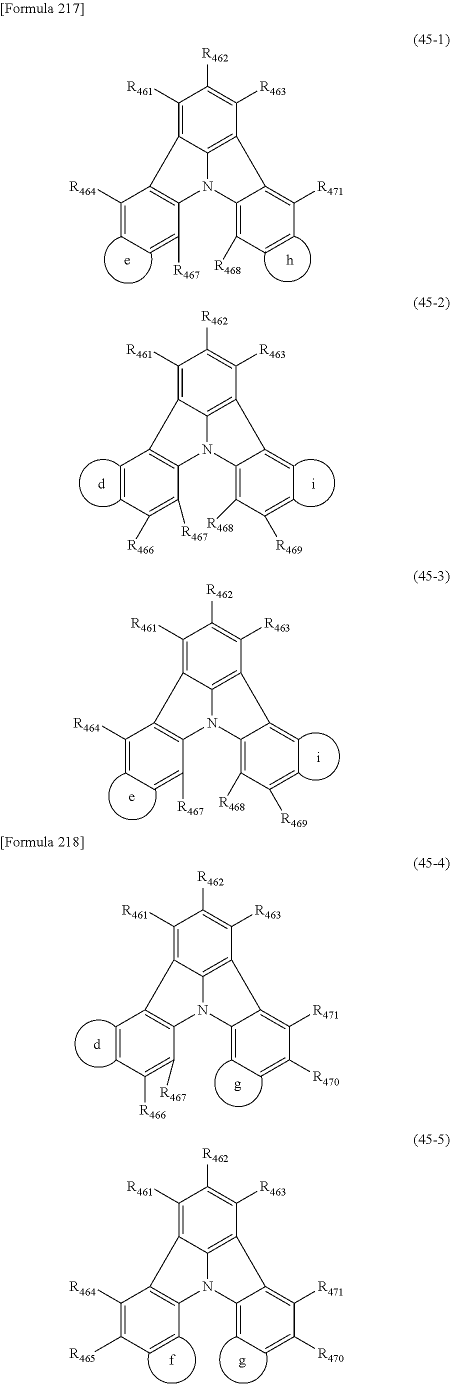

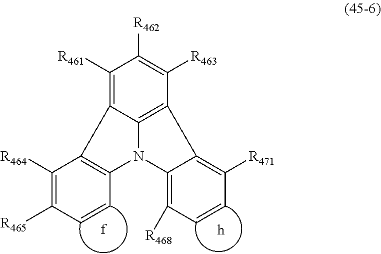

In the formulae (45-1) to (45-6):
rings d to i are each dependently a substituted or unsubstituted monocyclic ring or a substituted or unsubstituted fused ring; and
R₄₆₁ to R₄₇₁ each independently represent the same as R₄₆₁ to R₄₇₁ in the formula (45).

In an exemplary embodiment, the compound represented by the formula (45) is represented by one of formulae (45-7) to (45-12) below.

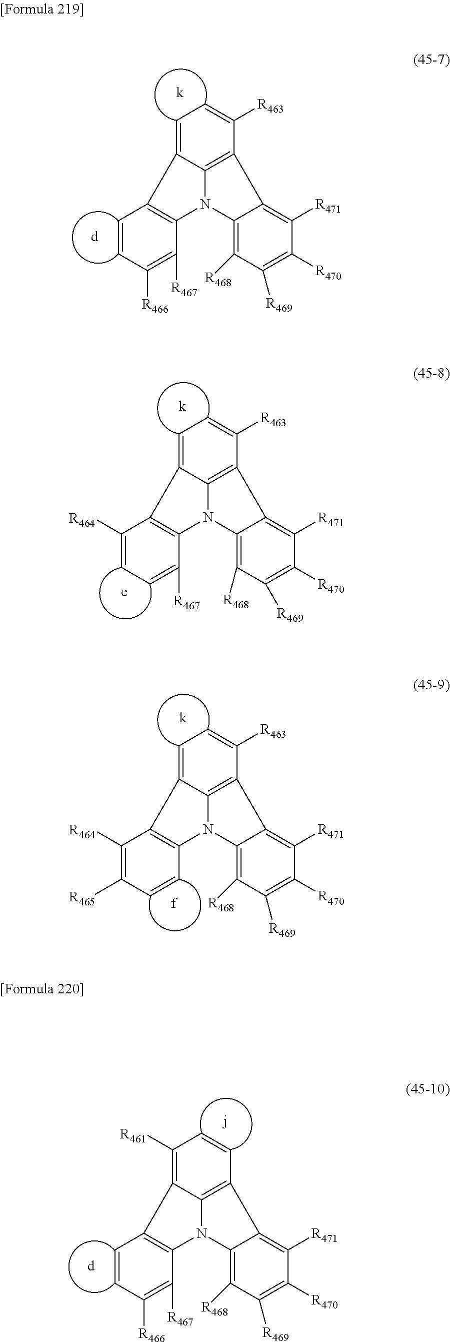

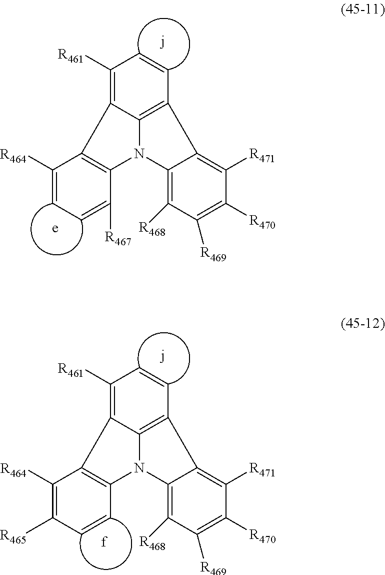

In the formulae (45-7) to (45-12):
rings d to f, k and j are each dependently a substituted or unsubstituted monocyclic ring or a substituted or unsubstituted fused ring; and
R₄₆₁ to R₄₇₁ each independently represent the same as R₄₆₁ to R₄₇₁ in the formula (45).

In an exemplary embodiment, the compound represented by the formula (45) is represented by one of formulae (45-13) to (45-21) below.

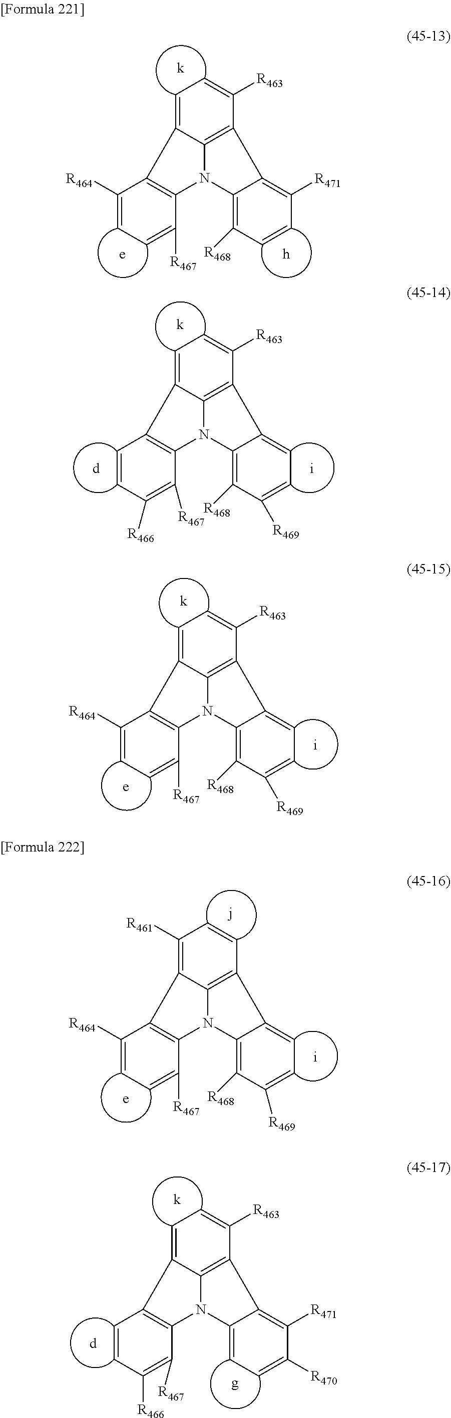

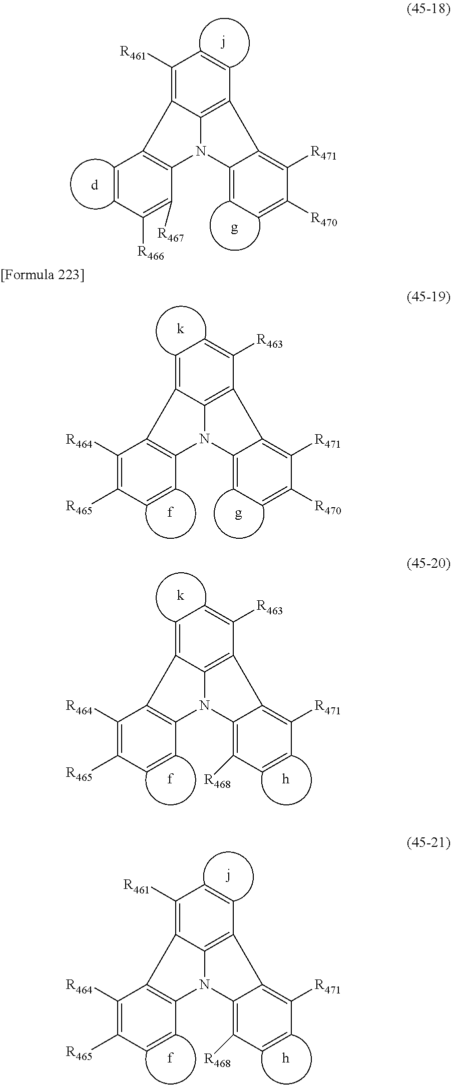

In the formulae (45-13) to (45-21):
rings d to k are each dependently a substituted or unsubstituted monocyclic ring or a substituted or unsubstituted fused ring, and
R₄₆₁ to R₄₇₁ each independently represent the same as R₄₆₁ to R₄₇₁ in the formula (45).

When the ring g or the ring h further has a substituent, examples of the substituent include a substituted or unsubstituted alkyl group having 1 to 50 carbon atoms, a substituted or unsubstituted aryl group having 6 to 50 ring carbon atoms, a group represented by the formula (461), a group represented by the formula (463), and a group represented by the formula (464).

In an exemplary embodiment, the compound represented by the formula (45) is represented by one of formulae (45-22) to (45-25) below.

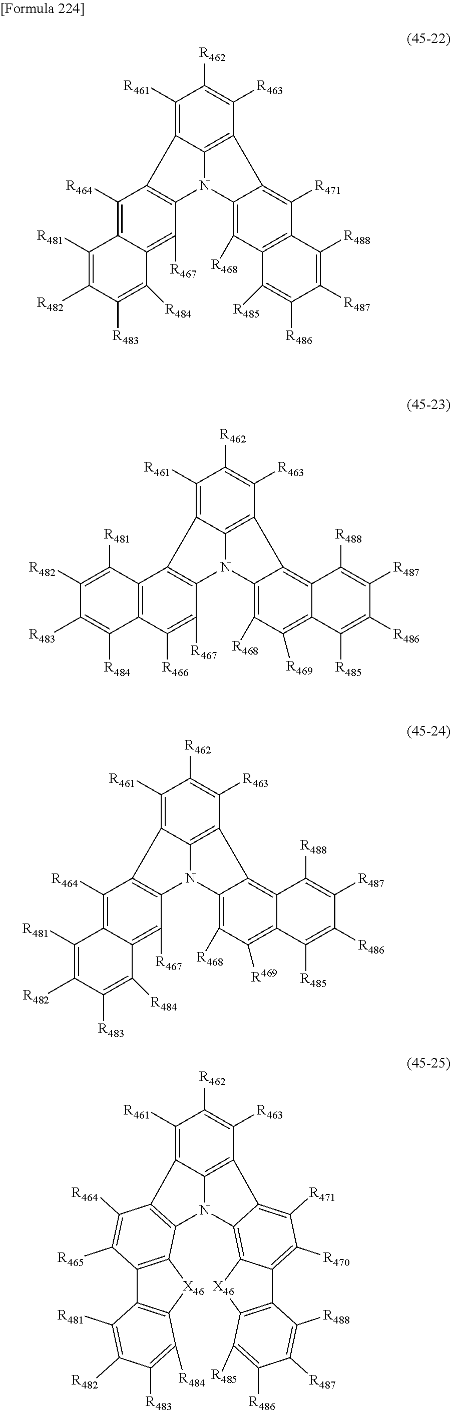

In the formulae (45-22) to (45-25):
X₄₆ and X₄₇ are each independently G(R₈₀₁)(R₈₀₂), NR₈₀₃, an oxygen atom or a sulfur atom;
R₄₆₁ to R₄₇₁ and R₄₈₁ to R₄₈₈ respectively represent the same as R₄₆₁ to R₄₇₁ of the formula (45);
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, a substituted or unsubstituted heterocyclic group having 5 to 50 ring atoms;
a substituted or unsubstituted alkyl group having 1 to 50 carbon atoms, a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

In an exemplary embodiment, the compound represented by the formula (45) is represented by a formula (45-26) below.

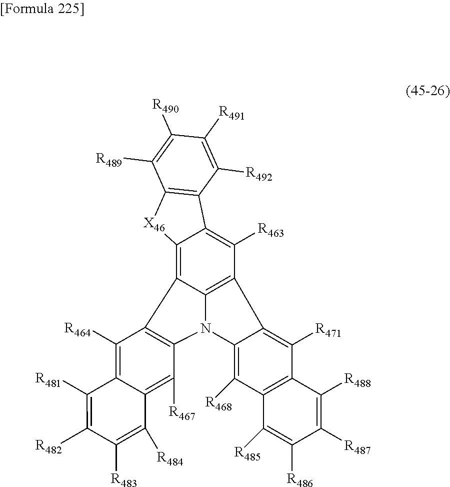

In the formula (45-26): X₄₆ represents C(R₈₀₁)(R₈₀₂), NR₈₀₃, an oxygen atom or a sulfur atom;
R₄₆₃, R₄₆₄, R₄₆₇, R₄₆₈, R₄₇₁, and R₄₈₁ to R₄₉₂ each independently represent the same as R₄₆₁ to R₄₇₁ in the formula (45);
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

### Specific Examples of Compound Represented by Formula (4)

Specific examples of the compound represented by the formula (4) include compounds shown below. In the specific examples below, Ph represents a phenyl group, and D represents a deuterium atom.

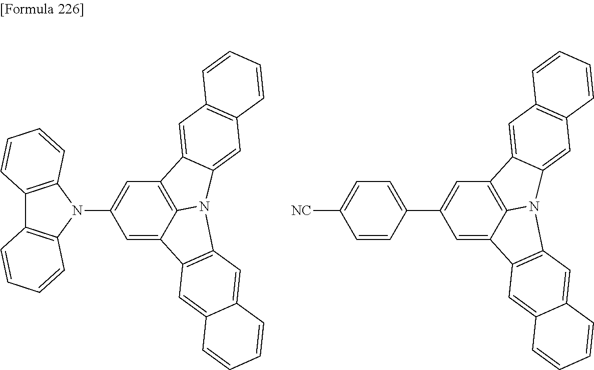 [SMILES: c1ccc2cc3c(cc2c1)c1cc(-n2c4ccccc4c4ccccc42)cc2c4cc5ccccc5cc4n3c12 ; N#Cc1ccc(-c2cc3c4cc5ccccc5cc4n4c5cc6ccccc6cc5c(c2)c34)cc1]

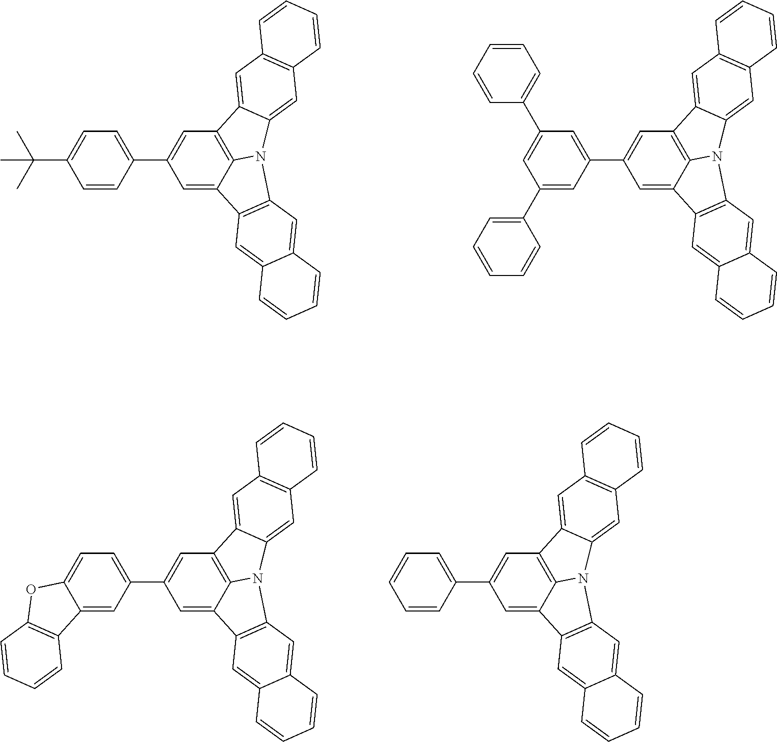 [SMILES: CC(C)(C)c1ccc(-c2cc3c4cc5ccccc5cc4n4c5cc6ccccc6cc5c(c2)c34)cc1 ; c1ccc(-c2cc(-c3ccccc3)cc(-c3cc4c5cc6ccccc6cc5n5c6cc7ccccc7cc6c(c3)c45)c2)cc1 ; c1ccc2cc3c(cc2c1)c1cc(-c2ccc4oc5ccccc5c4c2)cc2c4cc5ccccc5cc4n3c12 ; c1ccc(-c2cc3c4cc5ccccc5cc4n4c5cc6ccccc6cc5c(c2)c34)cc1]

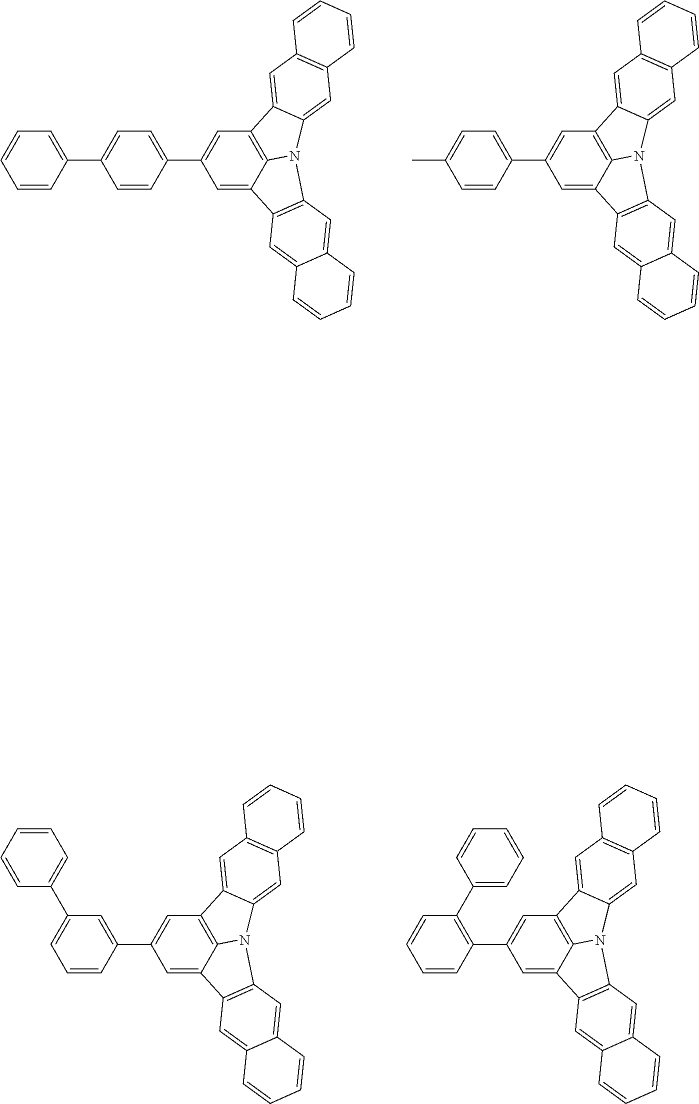 [SMILES: c1ccc(-c2ccc(-c3cc4c5cc6ccccc6cc5n5c6cc7ccccc7cc6c(c3)c45)cc2)cc1 ; Cc1ccc(-c2cc3c4cc5ccccc5cc4n4c5cc6ccccc6cc5c(c2)c34)cc1 ; c1ccc(-c2cccc(-c3cc4c5cc6ccccc6cc5n5c6cc7ccccc7cc6c(c3)c45)c2)cc1 ; c1ccc(-c2ccccc2-c2cc3c4cc5ccccc5cc4n4c5cc6ccccc6cc5c(c2)c34)cc1]

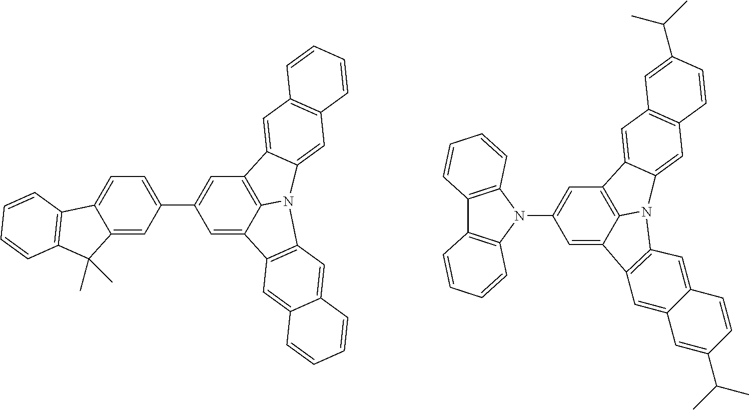 [SMILES: CC1(C)c2ccccc2-c2ccc(-c3cc4c5cc6ccccc6cc5n5c6cc7ccccc7cc6c(c3)c45)cc21 ; CC(C)c1ccc2cc3c(cc2c1)c1cc(-n2c4ccccc4c4ccccc42)cc2c4cc5cc(C(C)C)ccc5cc4n3c12]

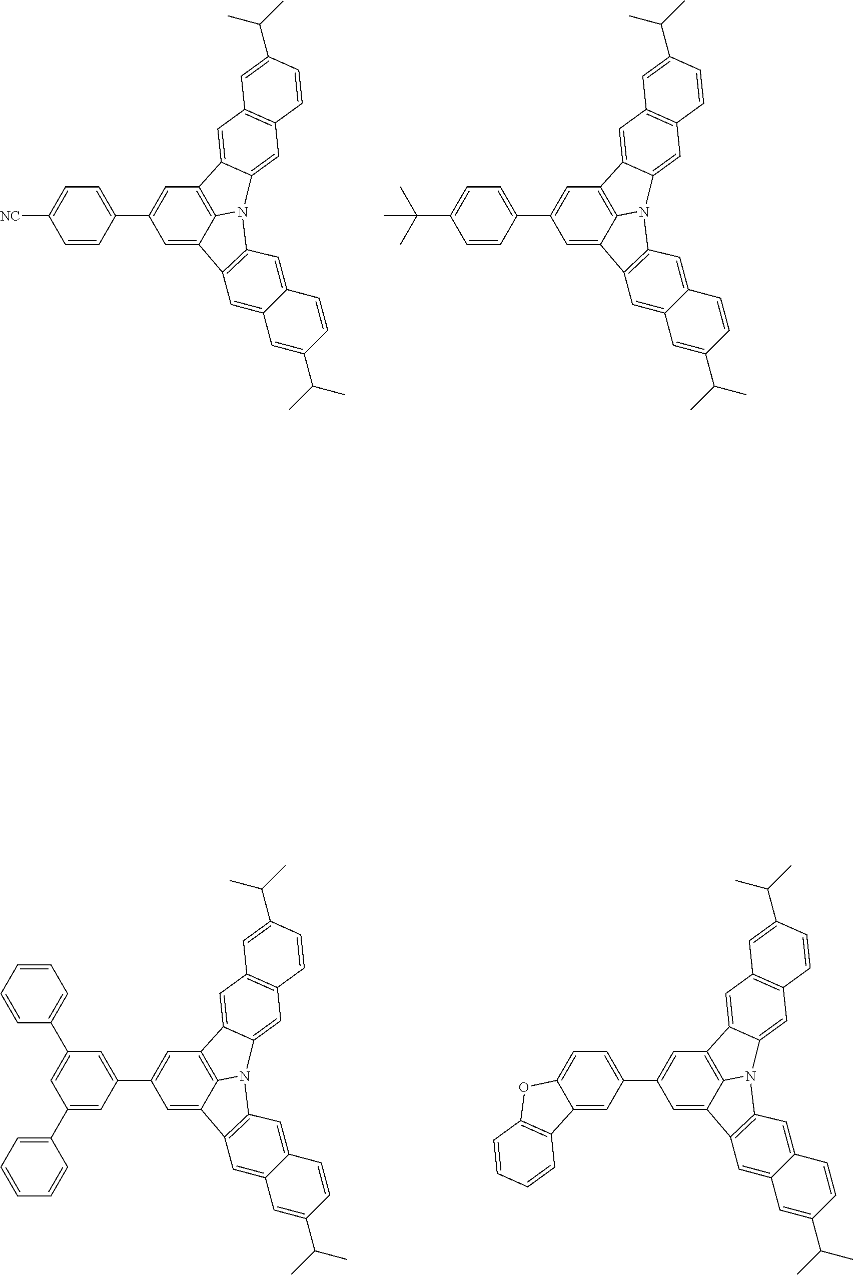 [SMILES: CC(C)c1ccc2cc3c(cc2c1)c1cc(-c2ccc(C#N)cc2)cc2c4cc5cc(C(C)C)ccc5cc4n3c12 ; CC(C)c1ccc2cc3c(cc2c1)c1cc(-c2ccc(C(C)(C)C)cc2)cc2c4cc5cc(C(C)C)ccc5cc4n3c12 ; CC(C)c1ccc2cc3c(cc2c1)c1cc(-c2cc(-c4ccccc4)cc(-c4ccccc4)c2)cc2c4cc5cc(C(C)C)ccc5cc4n3c12 ; CC(C)c1ccc2cc3c(cc2c1)c1cc(-c2ccc4oc5ccccc5c4c2)cc2c4cc5cc(C(C)C)ccc5cc4n3c12]

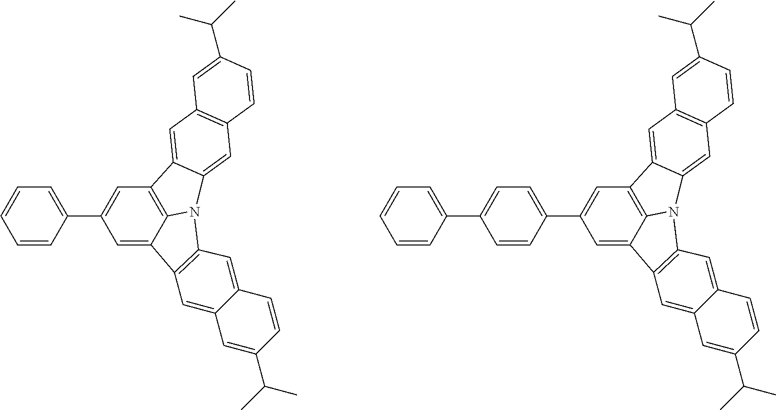 [SMILES: CC(C)c1ccc2cc3c(cc2c1)c1cc(-c2ccccc2)cc2c4cc5cc(C(C)C)ccc5cc4n3c12 ; CC(C)c1ccc2cc3c(cc2c1)c1cc(-c2ccc(-c4ccccc4)cc2)cc2c4cc5cc(C(C)C)ccc5cc4n3c12]

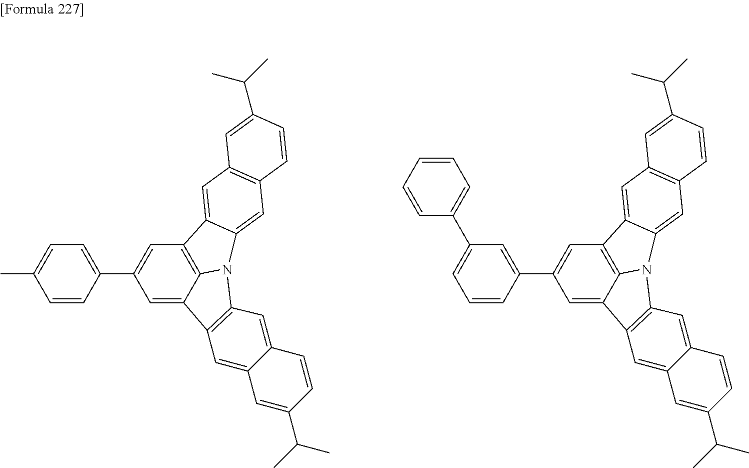 [SMILES: Cc1ccc(-c2cc3c4cc5cc(C(C)C)ccc5cc4n4c5cc6ccc(C(C)C)cc6cc5c(c2)c34)cc1 ; CC(C)c1ccc2cc3c(cc2c1)c1cc(-c2cccc(-c4ccccc4)c2)cc2c4cc5cc(C(C)C)ccc5cc4n3c12]

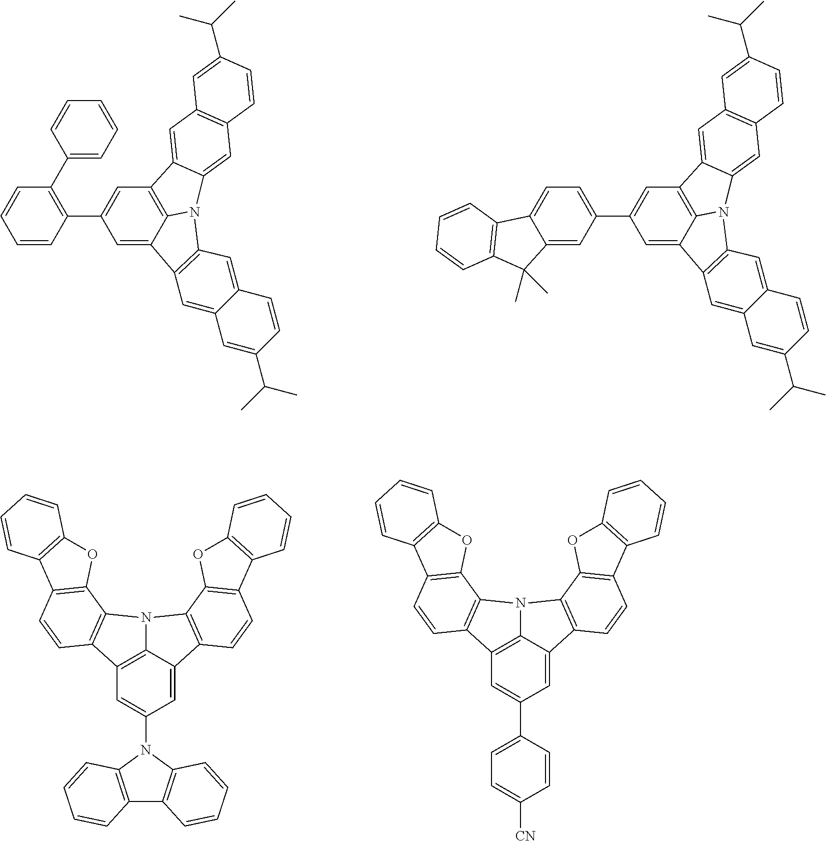 [SMILES: CC(C)c1ccc2cc3c(cc2c1)c1cc(-c2ccccc2-c2ccccc2)cc2c4cc5cc(C(C)C)ccc5cc4n3c12 ; CC(C)c1ccc2cc3c(cc2c1)c1cc(-c2ccc4c(c2)C(C)(C)c2ccccc2-4)cc2c4cc5cc(C(C)C)ccc5cc4n3c12 ; c1ccc2c(c1)oc1c2ccc2c3cc(-n4c5ccccc5c5ccccc54)cc4c5ccc6c7ccccc7oc6c5n(c34)c21 ; N#Cc1ccc(-c2cc3c4ccc5c6ccccc6oc5c4n4c3c(c2)c2ccc3c5ccccc5oc3c24)cc1]

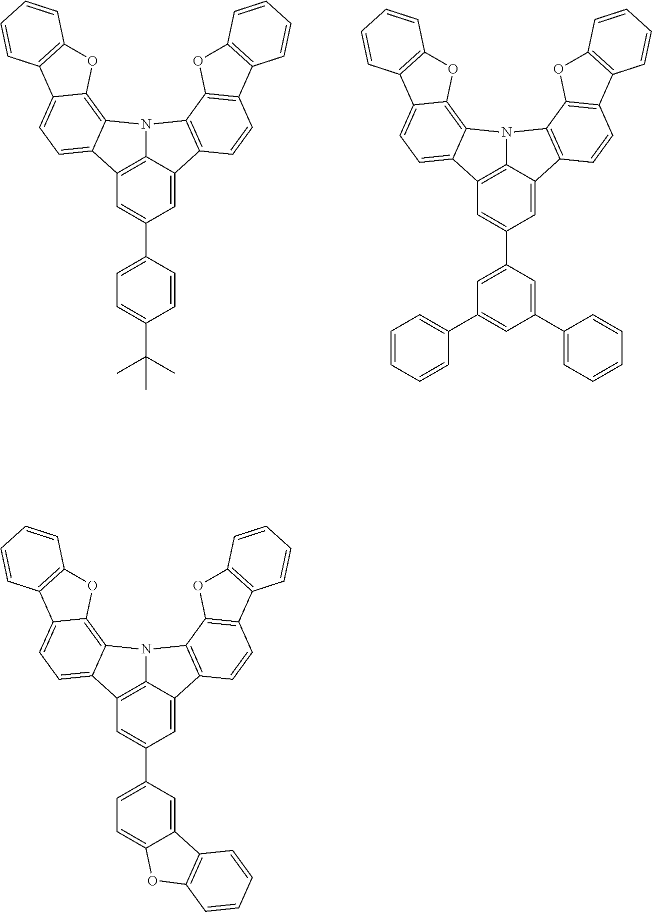 [SMILES: CC(C)(C)c1ccc(-c2cc3c4ccc5c6ccccc6oc5c4n4c3c(c2)c2ccc3c5ccccc5oc3c24)cc1 ; c1ccc(-c2cc(-c3ccccc3)cc(-c3cc4c5ccc6c7ccccc7oc6c5n5c4c(c3)c3ccc4c6ccccc6oc4c35)c2)cc1 ; c1ccc2c(c1)oc1ccc(-c3cc4c5ccc6c7ccccc7oc6c5n5c4c(c3)c3ccc4c6ccccc6oc4c35)cc12]

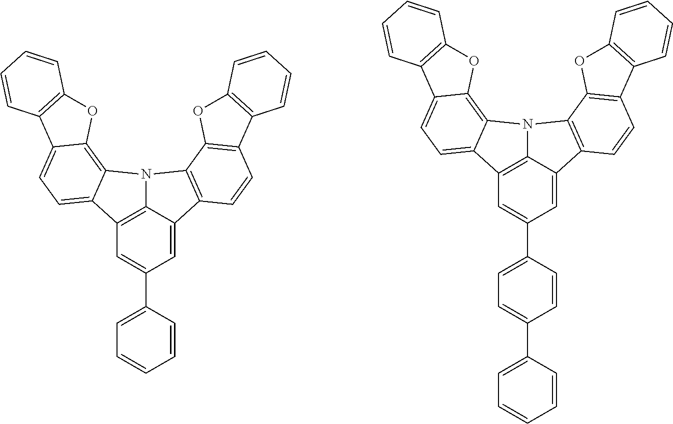 [SMILES: c1ccc(-c2cc3c4ccc5c6ccccc6oc5c4n4c3c(c2)c2ccc3c5ccccc5oc3c24)cc1 ; c1ccc(-c2ccc(-c3cc4c5ccc6c7ccccc7oc6c5n5c4c(c3)c3ccc4c6ccccc6oc4c35)cc2)cc1]

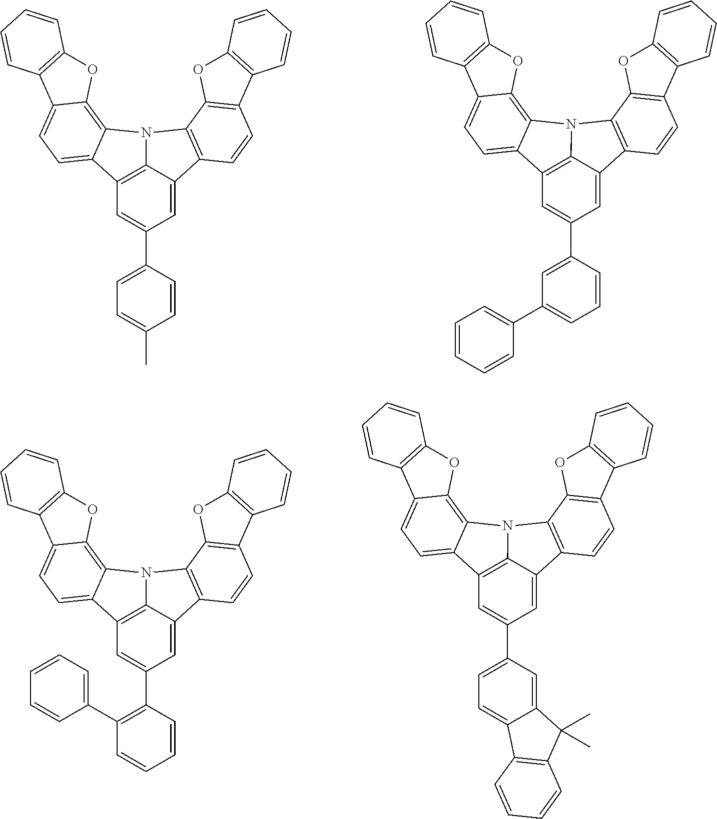 [SMILES: Cc1ccc(-c2cc3c4ccc5c6ccccc6oc5c4n4c3c(c2)c2ccc3c5ccccc5oc3c24)cc1 ; c1ccc(-c2cccc(-c3cc4c5ccc6c7ccccc7oc6c5n5c4c(c3)c3ccc4c6ccccc6oc4c35)c2)cc1 ; c1ccc(-c2ccccc2-c2cc3c4ccc5c6ccccc6oc5c4n4c3c(c2)c2ccc3c5ccccc5oc3c24)cc1 ; CC1(C)c2ccccc2-c2ccc(-c3cc4c5ccc6c7ccccc7oc6c5n5c4c(c3)c3ccc4c6ccccc6oc4c35)cc21]

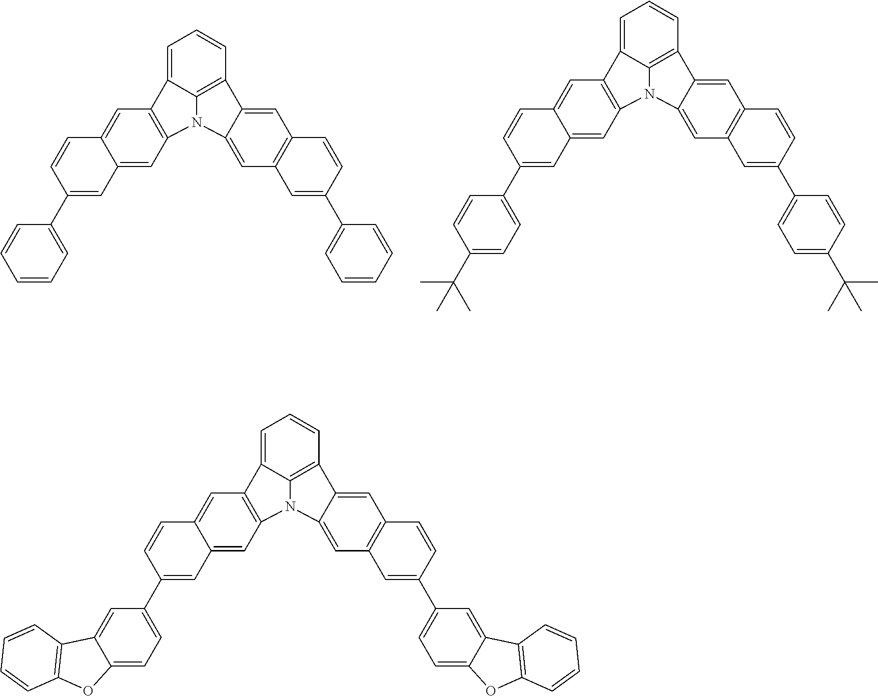

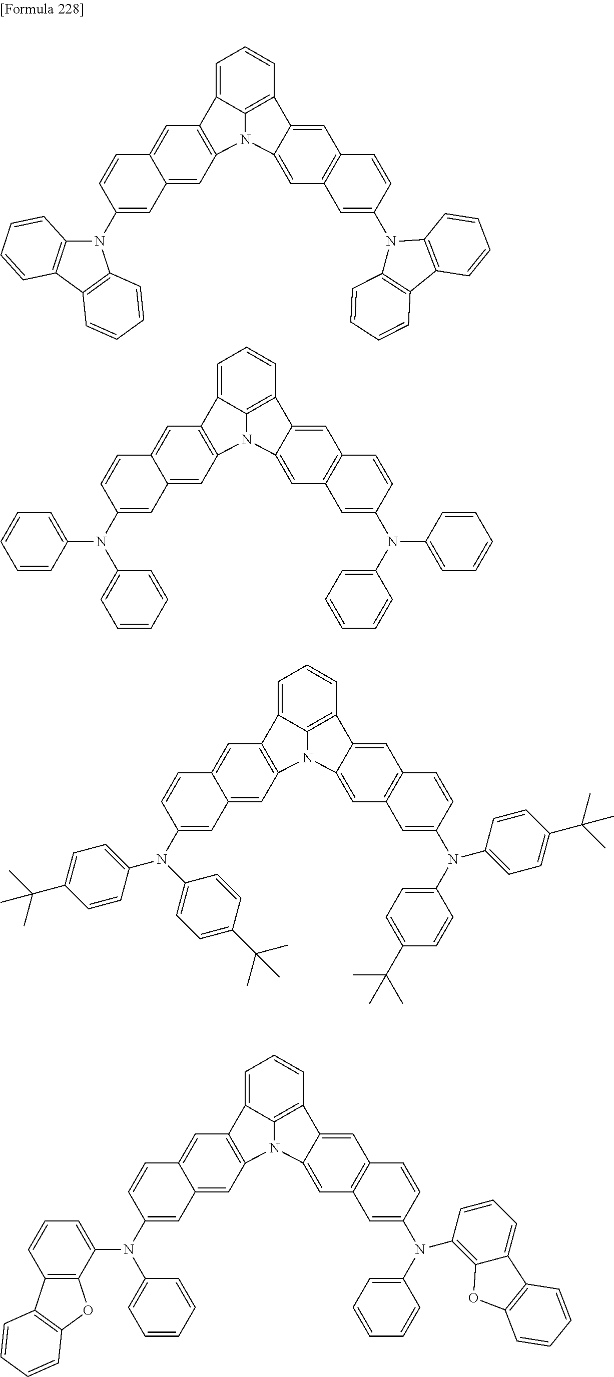 [SMILES: c1ccc2c(c1)c1ccccc1n2-c1ccc2cc3c4cccc5c6cc7ccc(-n8c9ccccc9c9ccccc98)cc7cc6n(c3cc2c1)c45 ; c1ccc(N(c2ccccc2)c2ccc3cc4c5cccc6c7cc8ccc(N(c9ccccc9)c9ccccc9)cc8cc7n(c4cc3c2)c56)cc1 ; CC(C)(C)c1ccc(N(c2ccc(C(C)(C)C)cc2)c2ccc3cc4c5cccc6c7cc8ccc(N(c9ccc(C(C)(C)C)cc9)c9ccc(C(C)(C)C)cc9)cc8cc7n(c4cc3c2)c56)cc1 ; c1ccc(N(c2ccc3cc4c5cccc6c7cc8ccc(N(c9ccccc9)c9cccc%10c9oc9ccccc9%10)cc8cc7n(c4cc3c2)c56)c2cccc3c2oc2ccccc23)cc1]

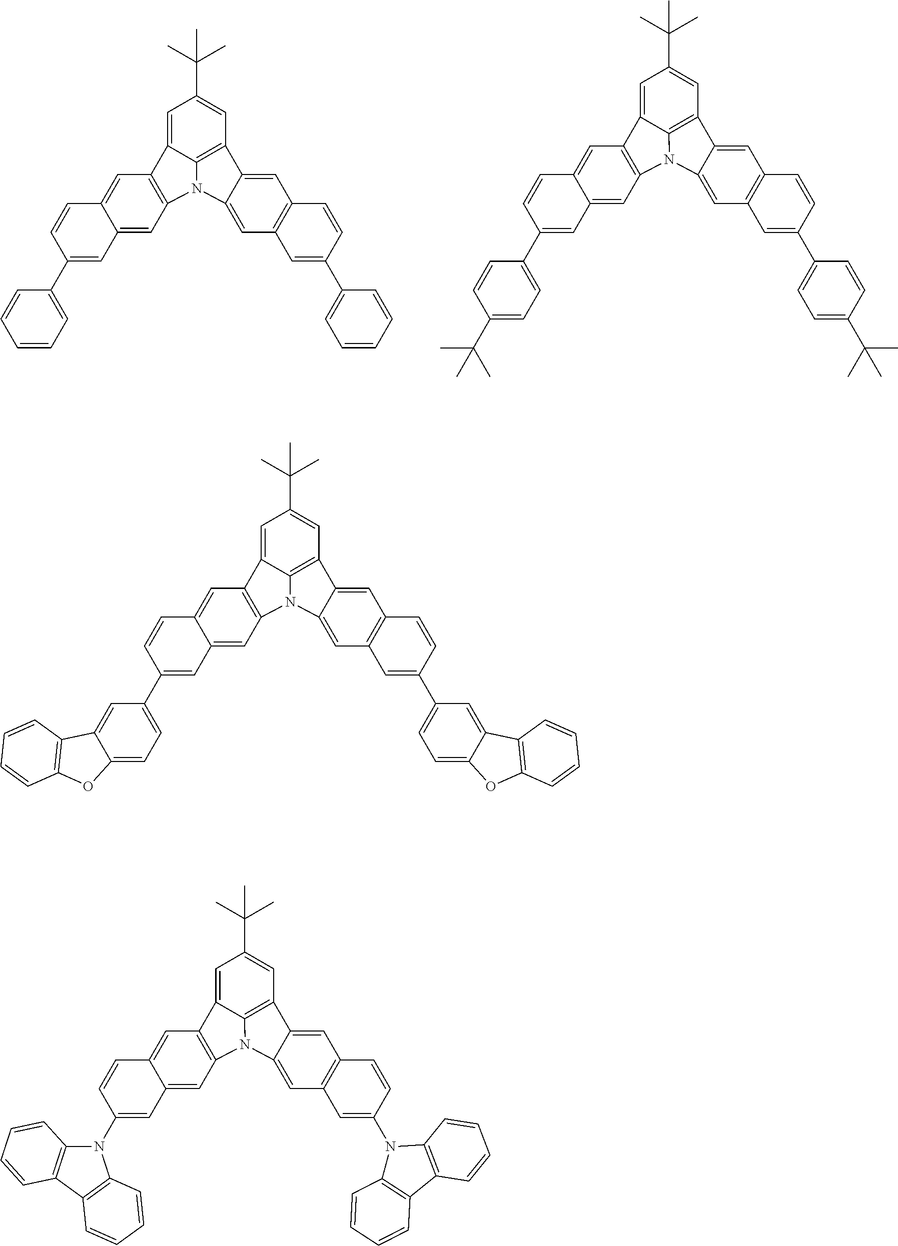 [SMILES: CC(C)(C)c1cc2c3cc4ccc(-c5ccccc5)cc4cc3n3c4cc5cc(-c6ccccc6)ccc5cc4c(c1)c23 ; CC(C)(C)c1ccc(-c2ccc3cc4c5cc(C(C)(C)C)cc6c7cc8ccc(-c9ccc(C(C)(C)C)cc9)cc8cc7n(c4cc3c2)c56)cc1 ; CC(C)(C)c1cc2c3cc4ccc(-c5ccc6oc7ccccc7c6c5)cc4cc3n3c4cc5cc(-c6ccc7oc8ccccc8c7c6)ccc5cc4c(c1)c23 ; CC(C)(C)c1cc2c3cc4ccc(-n5c6ccccc6c6ccccc65)cc4cc3n3c4cc5cc(-n6c7ccccc7c7ccccc76)ccc5cc4c(c1)c23]

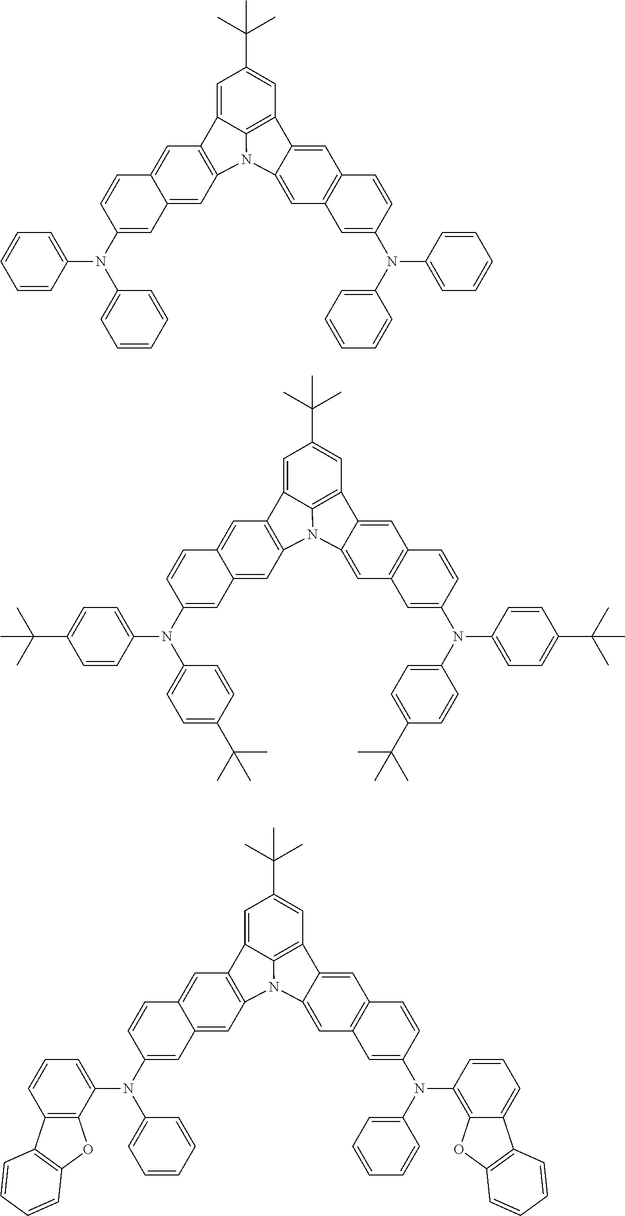

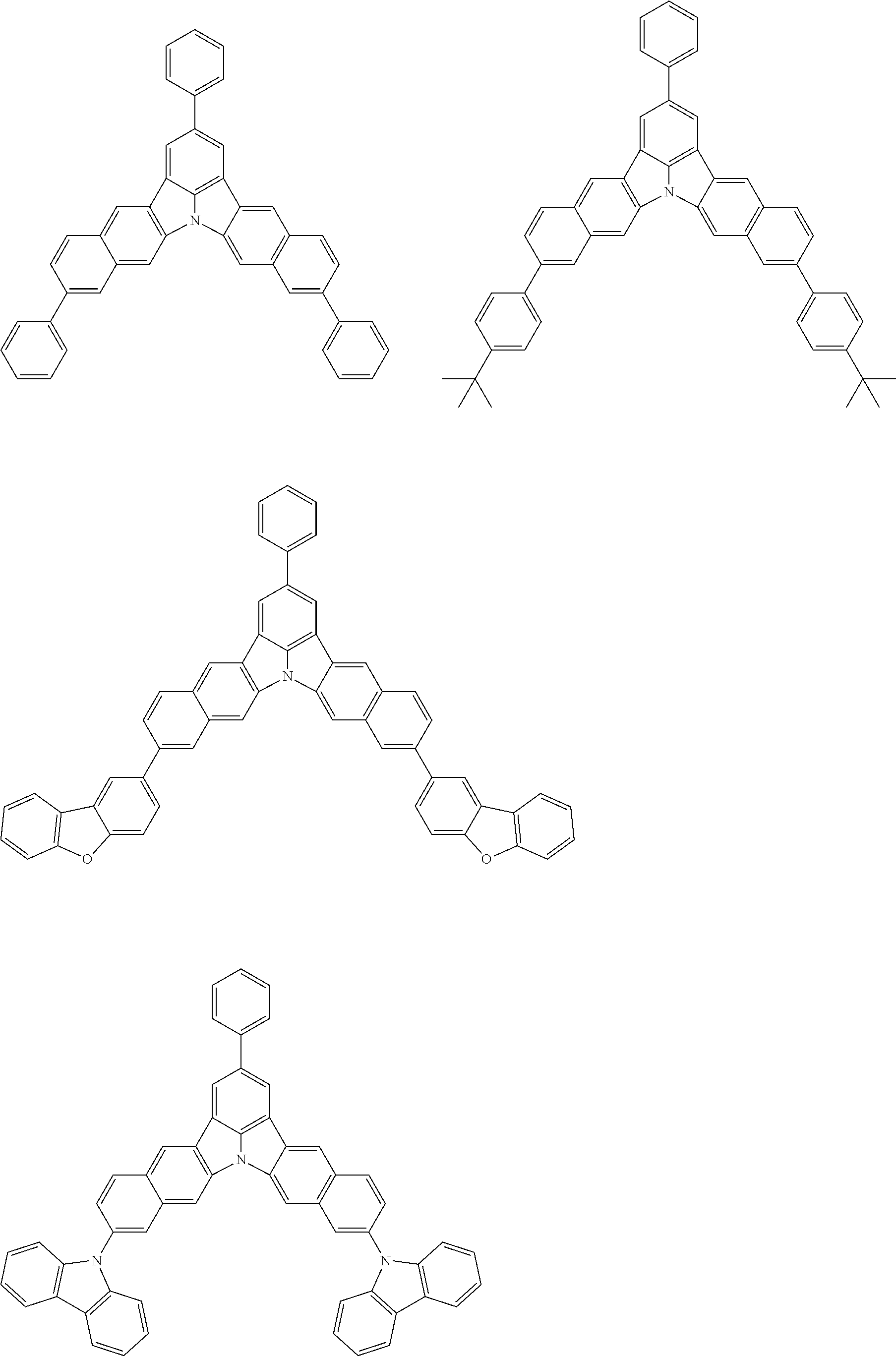 [SMILES: c1ccc(-c2ccc3cc4c5cc(-c6ccccc6)cc6c7cc8ccc(-c9ccccc9)cc8cc7n(c4cc3c2)c56)cc1 ; CC(C)(C)c1ccc(-c2ccc3cc4c5cc(-c6ccccc6)cc6c7cc8ccc(-c9ccc(C(C)(C)C)cc9)cc8cc7n(c4cc3c2)c56)cc1 ; c1ccc(-c2cc3c4cc5ccc(-c6ccc7oc8ccccc8c7c6)cc5cc4n4c5cc6cc(-c7ccc8oc9ccccc9c8c7)ccc6cc5c(c2)c34)cc1 ; c1ccc(-c2cc3c4cc5ccc(-n6c7ccccc7c7ccccc76)cc5cc4n4c5cc6cc(-n7c8ccccc8c8ccccc87)ccc6cc5c(c2)c34)cc1]

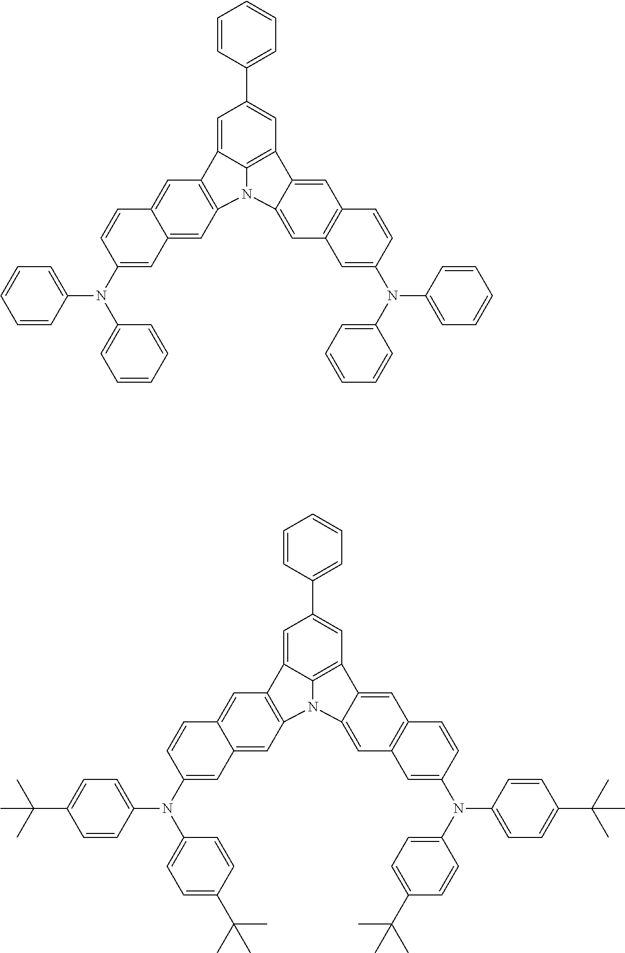 [SMILES: c1ccc(-c2cc3c4cc5ccc(N(c6ccccc6)c6ccccc6)cc5cc4n4c5cc6cc(N(c7ccccc7)c7ccccc7)ccc6cc5c(c2)c34)cc1 ; CC(C)(C)c1ccc(N(c2ccc(C(C)(C)C)cc2)c2ccc3cc4c5cc(-c6ccccc6)cc6c7cc8ccc(N(c9ccc(C(C)(C)C)cc9)c9ccc(C(C)(C)C)cc9)cc8cc7n(c4cc3c2)c56)cc1]

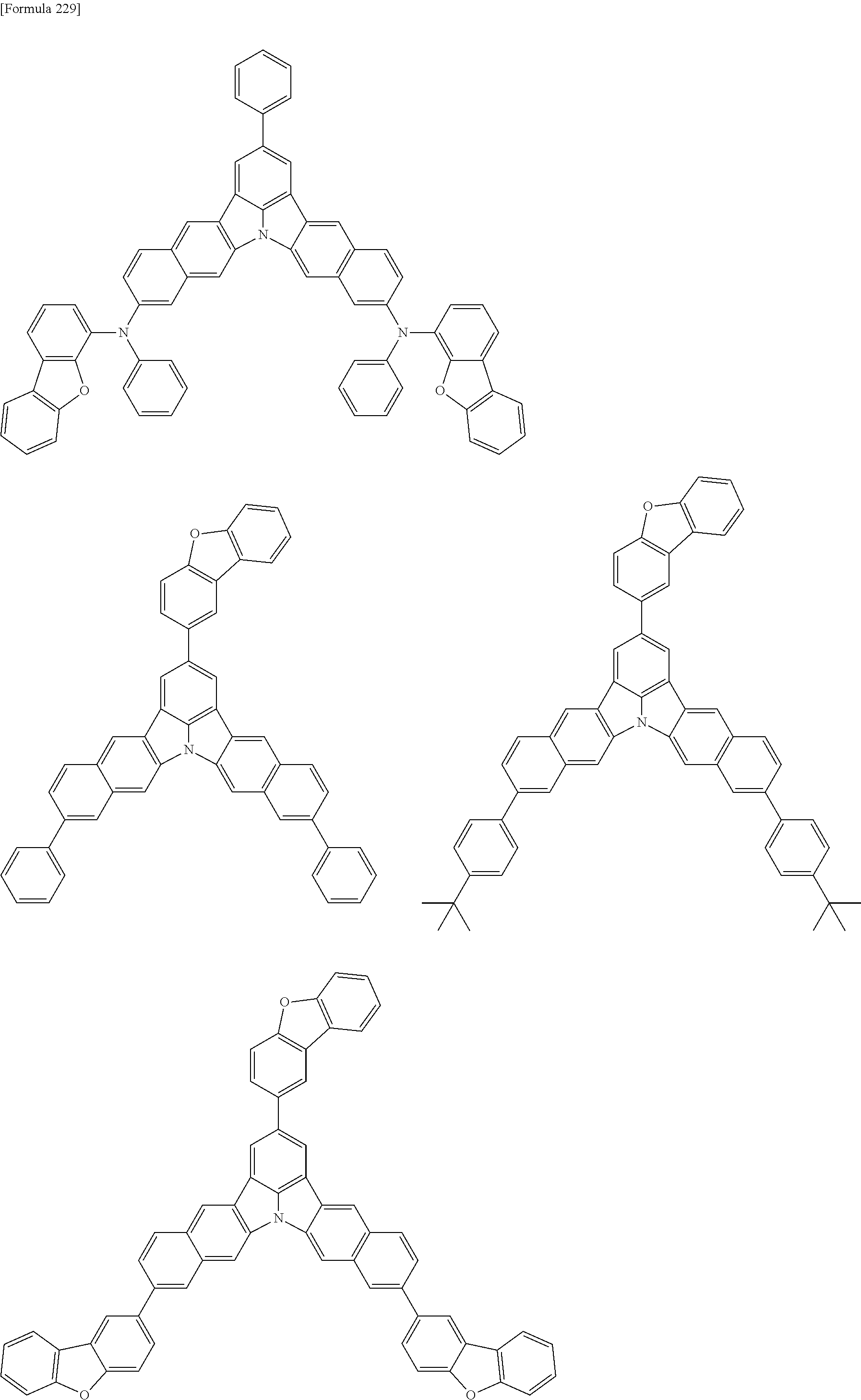 [SMILES: c1ccc(-c2cc3c4cc5ccc(N(c6ccccc6)c6cccc7c6oc6ccccc67)cc5cc4n4c5cc6cc(N(c7ccccc7)c7cccc8c7oc7ccccc78)ccc6cc5c(c2)c34)cc1 ; c1ccc(-c2ccc3cc4c5cc(-c6ccc7oc8ccccc8c7c6)cc6c7cc8ccc(-c9ccccc9)cc8cc7n(c4cc3c2)c56)cc1 ; CC(C)(C)c1ccc(-c2ccc3cc4c5cc(-c6ccc7oc8ccccc8c7c6)cc6c7cc8ccc(-c9ccc(C(C)(C)C)cc9)cc8cc7n(c4cc3c2)c56)cc1 ; c1ccc2c(c1)oc1ccc(-c3ccc4cc5c6cc(-c7ccc8oc9ccccc9c8c7)cc7c8cc9ccc(-c%10ccc%11oc%12ccccc%12c%11c%10)cc9cc8n(c5cc4c3)c67)cc12]

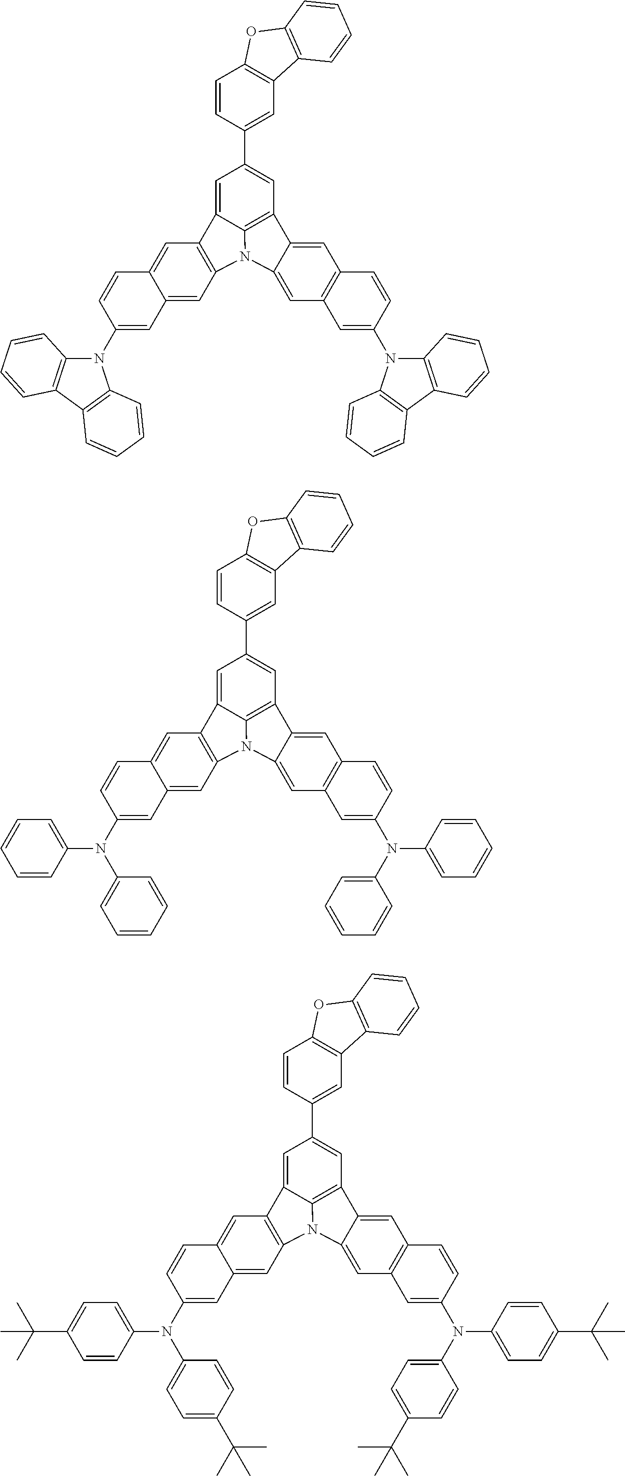

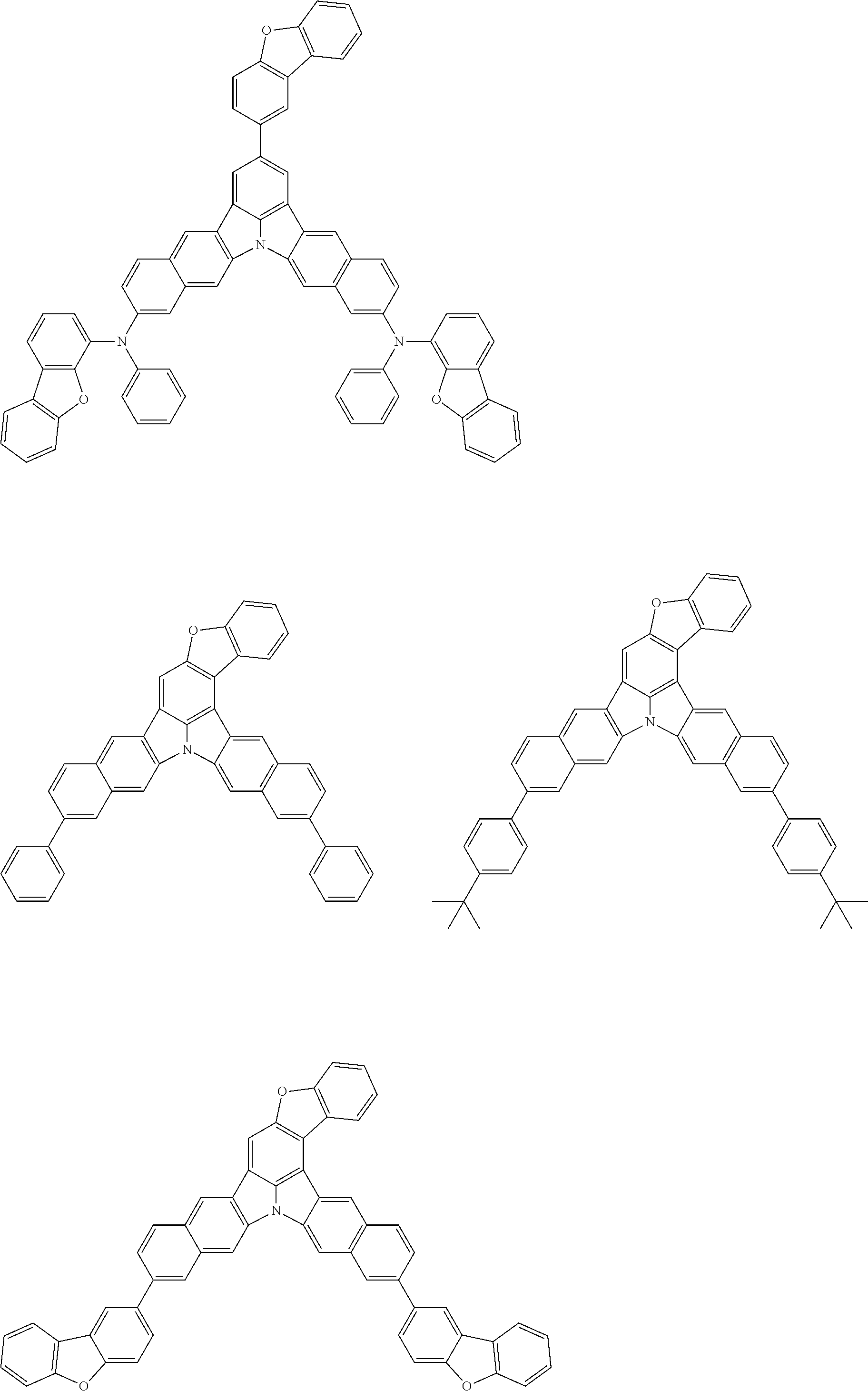 [SMILES: c1ccc(N(c2ccc3cc4c5cc(-c6ccc7oc8ccccc8c7c6)cc6c7cc8ccc(N(c9ccccc9)c9cccc%10c9oc9ccccc9%10)cc8cc7n(c4cc3c2)c56)c2cccc3c2oc2ccccc23)cc1 ; c1ccc(-c2ccc3cc4c5cc6oc7ccccc7c6c6c7cc8ccc(-c9ccccc9)cc8cc7n(c4cc3c2)c56)cc1 ; CC(C)(C)c1ccc(-c2ccc3cc4c5cc6oc7ccccc7c6c6c7cc8ccc(-c9ccc(C(C)(C)C)cc9)cc8cc7n(c4cc3c2)c56)cc1 ; c1ccc2c(c1)oc1ccc(-c3ccc4cc5c6cc7oc8ccccc8c7c7c8cc9ccc(-c%10ccc%11oc%12ccccc%12c%11c%10)cc9cc8n(c5cc4c3)c67)cc12]

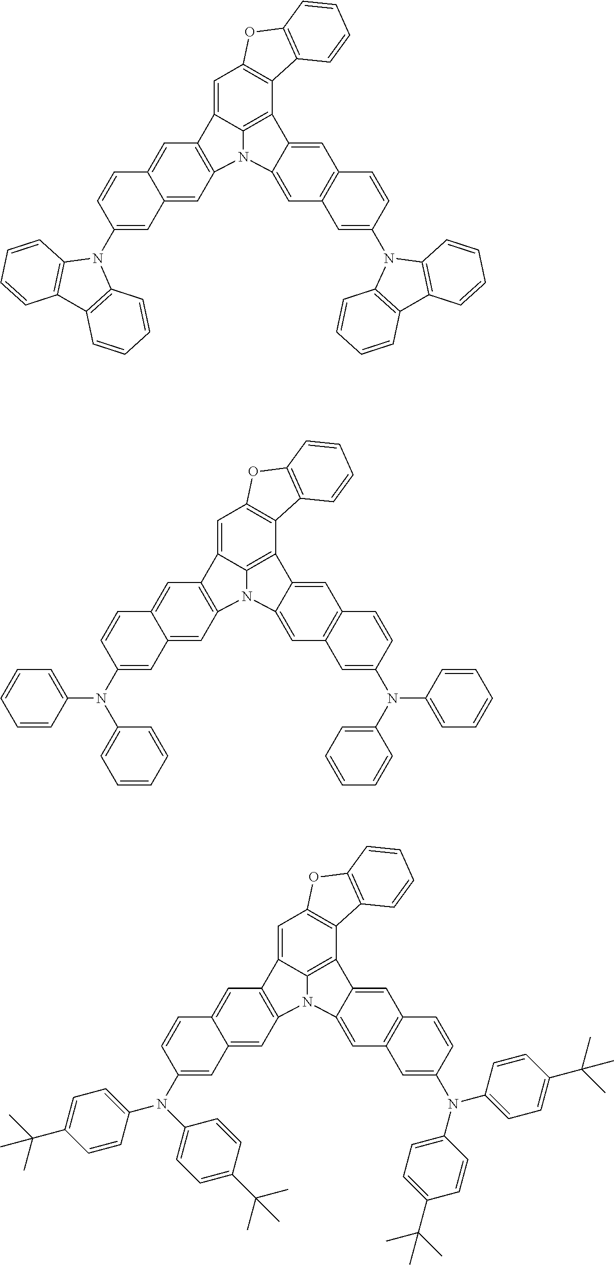 [SMILES: c1ccc2c(c1)oc1cc3c4cc5ccc(-n6c7ccccc7c7ccccc76)cc5cc4n4c5cc6cc(-n7c8ccccc8c8ccccc87)ccc6cc5c(c12)c34 ; c1ccc(N(c2ccccc2)c2ccc3cc4c5cc6oc7ccccc7c6c6c7cc8ccc(N(c9ccccc9)c9ccccc9)cc8cc7n(c4cc3c2)c56)cc1 ; CC(C)(C)c1ccc(N(c2ccc(C(C)(C)C)cc2)c2ccc3cc4c5cc6oc7ccccc7c6c6c7cc8ccc(N(c9ccc(C(C)(C)C)cc9)c9ccc(C(C)(C)C)cc9)cc8cc7n(c4cc3c2)c56)cc1]

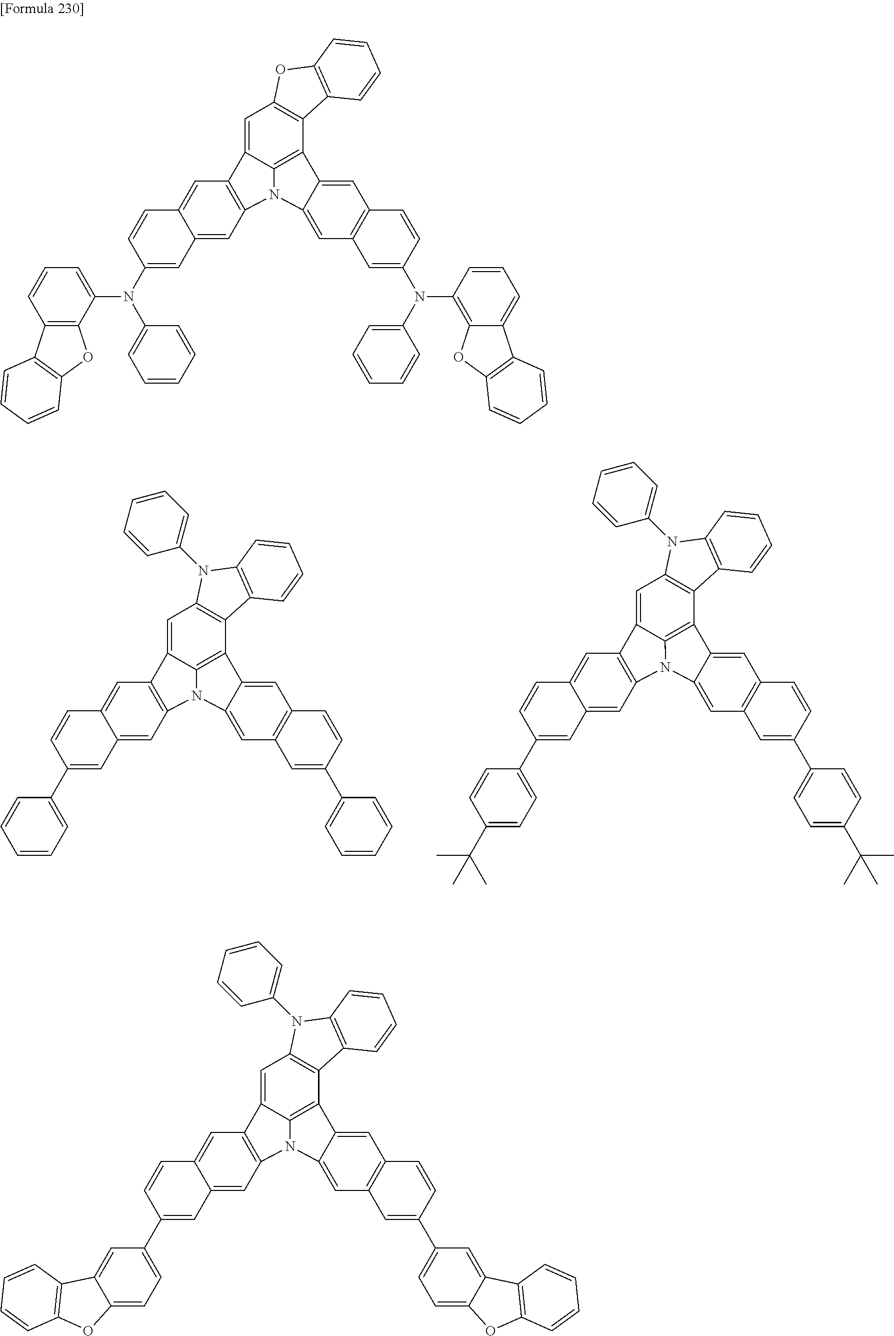 [SMILES: c1ccc(N(c2ccc3cc4c5cc6oc7ccccc7c6c6c7cc8ccc(N(c9ccccc9)c9cccc%10c9oc9ccccc9%10)cc8cc7n(c4cc3c2)c56)c2cccc3c2oc2ccccc23)cc1 ; c1ccc(-c2ccc3cc4c5cc6c(c7ccccc7n6-c6ccccc6)c6c7cc8ccc(-c9ccccc9)cc8cc7n(c4cc3c2)c56)cc1 ; CC(C)(C)c1ccc(-c2ccc3cc4c5cc6c(c7ccccc7n6-c6ccccc6)c6c7cc8ccc(-c9ccc(C(C)(C)C)cc9)cc8cc7n(c4cc3c2)c56)cc1 ; c1ccc(-n2c3ccccc3c3c4c5cc6ccc(-c7ccc8oc9ccccc9c8c7)cc6cc5n5c6cc7cc(-c8ccc9oc%10ccccc%10c9c8)ccc7cc6c(cc32)c45)cc1]

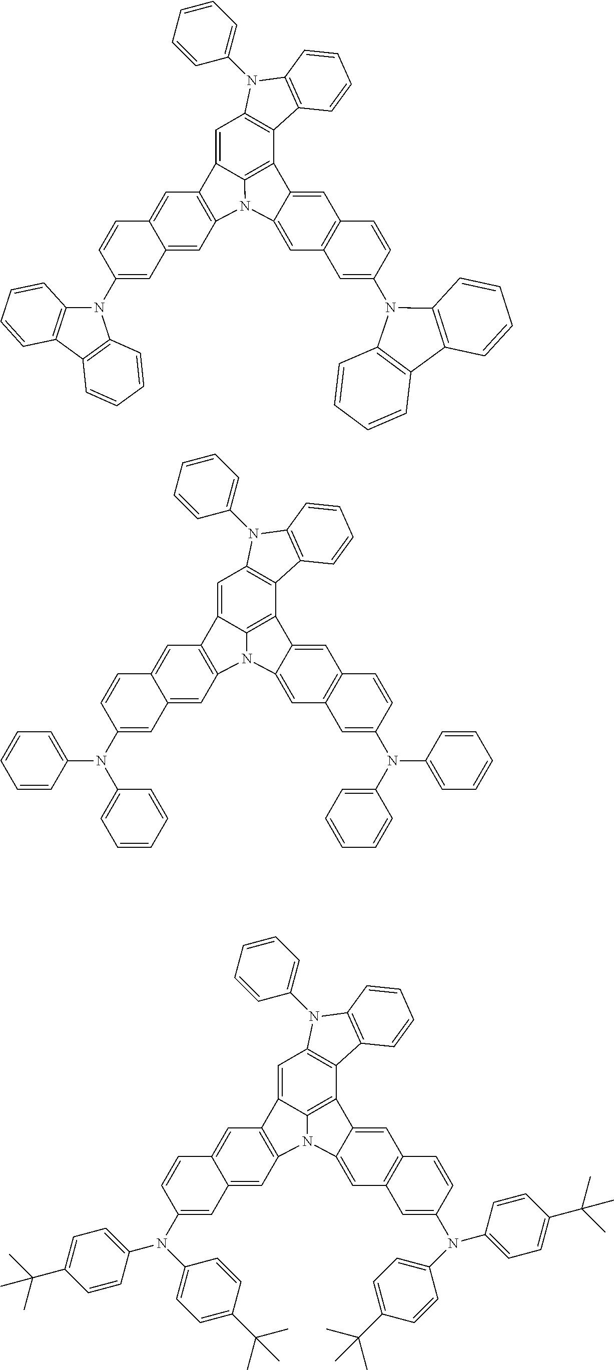 [SMILES: c1ccc(-n2c3ccccc3c3c4c5cc6ccc(-n7c8ccccc8c8ccccc87)cc6cc5n5c6cc7cc(-n8c9ccccc9c9ccccc98)ccc7cc6c(cc32)c45)cc1 ; c1ccc(N(c2ccccc2)c2ccc3cc4c5cc6c(c7ccccc7n6-c6ccccc6)c6c7cc8ccc(N(c9ccccc9)c9ccccc9)cc8cc7n(c4cc3c2)c56)cc1 ; CC(C)(C)c1ccc(N(c2ccc(C(C)(C)C)cc2)c2ccc3cc4c5cc6c(c7ccccc7n6-c6ccccc6)c6c7cc8ccc(N(c9ccc(C(C)(C)C)cc9)c9ccc(C(C)(C)C)cc9)cc8cc7n(c4cc3c2)c56)cc1]

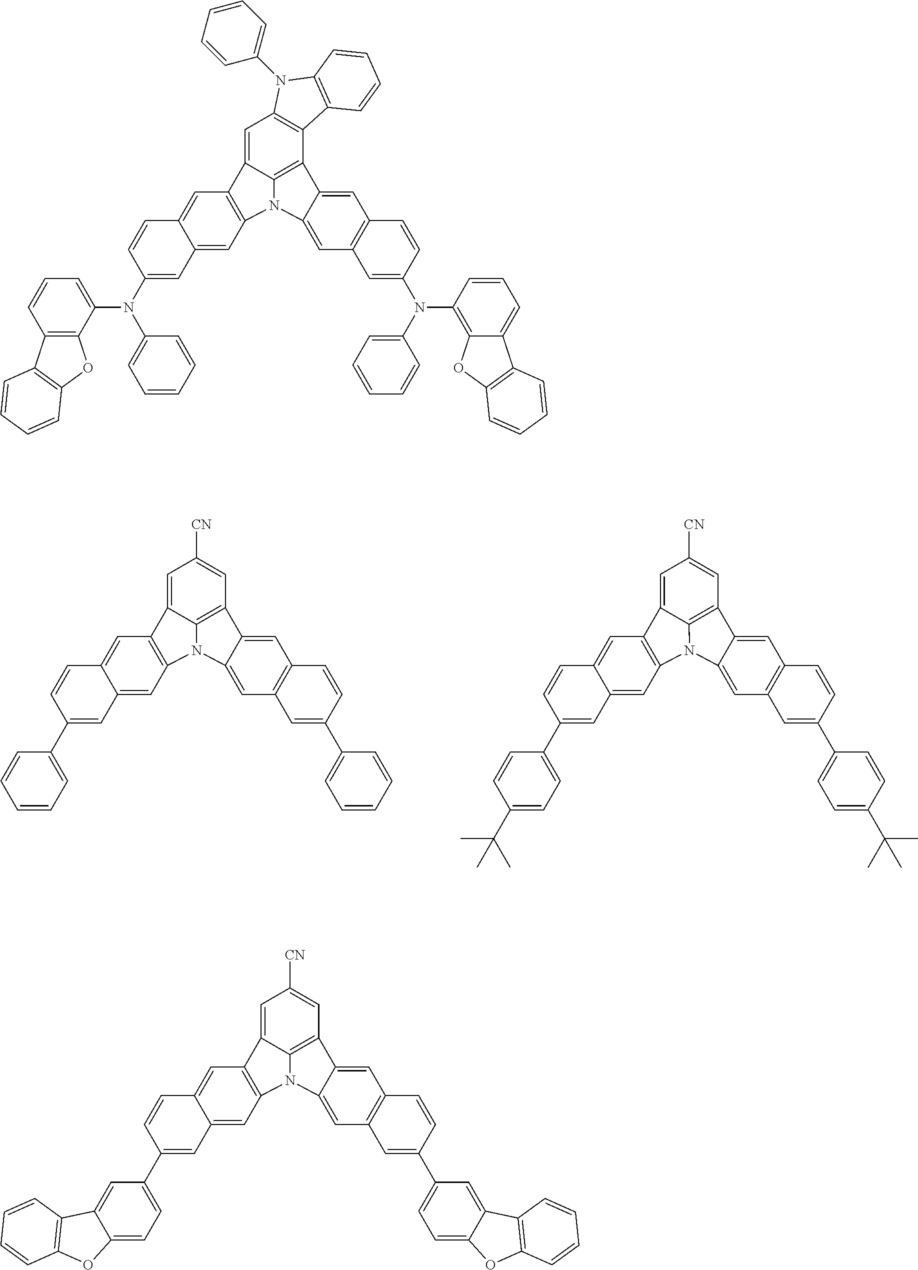 [SMILES: c1ccc(N(c2ccc3cc4c5cc6c(c7ccccc7n6-c6ccccc6)c6c7cc8ccc(N(c9ccccc9)c9cccc%10c9oc9ccccc9%10)cc8cc7n(c4cc3c2)c56)c2cccc3c2oc2ccccc23)cc1 ; N#Cc1cc2c3cc4ccc(-c5ccccc5)cc4cc3n3c4cc5cc(-c6ccccc6)ccc5cc4c(c1)c23 ; CC(C)(C)c1ccc(-c2ccc3cc4c5cc(C#N)cc6c7cc8ccc(-c9ccc(C(C)(C)C)cc9)cc8cc7n(c4cc3c2)c56)cc1 ; N#Cc1cc2c3cc4ccc(-c5ccc6oc7ccccc7c6c5)cc4cc3n3c4cc5cc(-c6ccc7oc8ccccc8c7c6)ccc5cc4c(c1)c23]

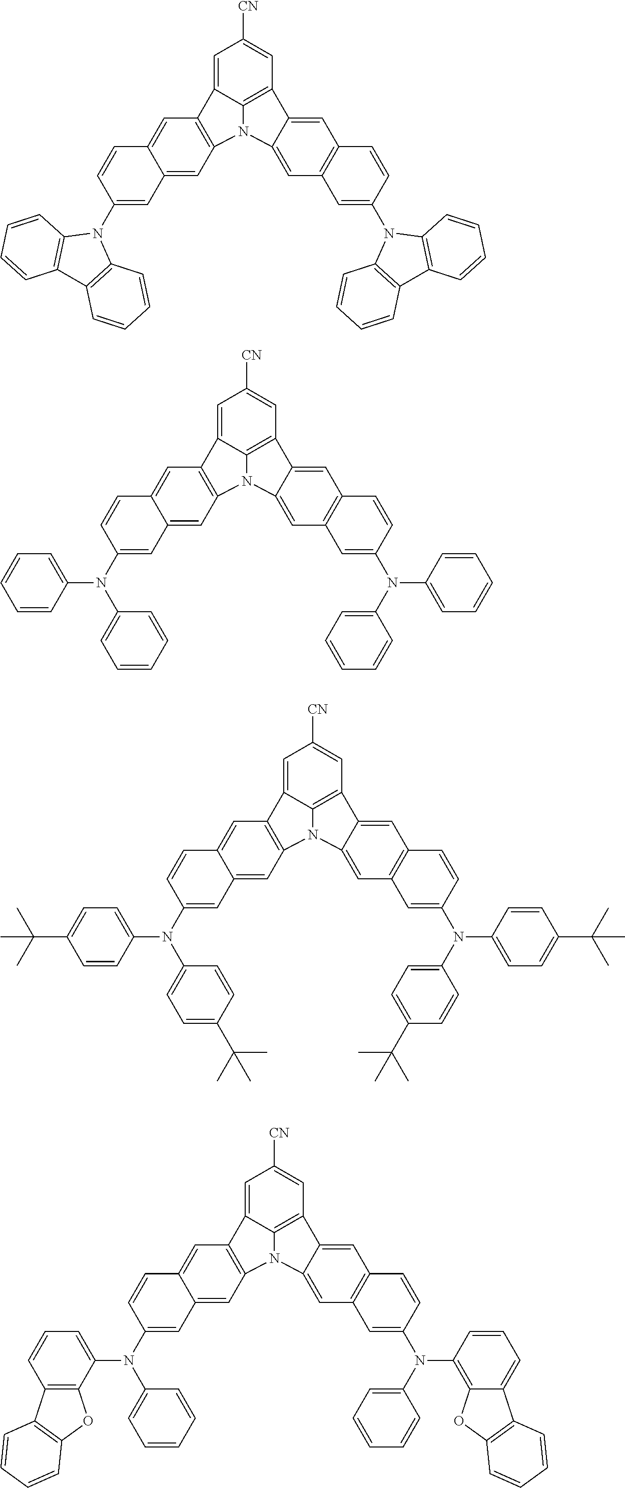 [SMILES: N#Cc1cc2c3cc4ccc(-n5c6ccccc6c6ccccc65)cc4cc3n3c4cc5cc(-n6c7ccccc7c7ccccc76)ccc5cc4c(c1)c23 ; N#Cc1cc2c3cc4ccc(N(c5ccccc5)c5ccccc5)cc4cc3n3c4cc5cc(N(c6ccccc6)c6ccccc6)ccc5cc4c(c1)c23 ; CC(C)(C)c1ccc(N(c2ccc(C(C)(C)C)cc2)c2ccc3cc4c5cc(C#N)cc6c7cc8ccc(N(c9ccc(C(C)(C)C)cc9)c9ccc(C(C)(C)C)cc9)cc8cc7n(c4cc3c2)c56)cc1 ; N#Cc1cc2c3cc4ccc(N(c5ccccc5)c5cccc6c5oc5ccccc56)cc4cc3n3c4cc5cc(N(c6ccccc6)c6cccc7c6oc6ccccc67)ccc5cc4c(c1)c23]

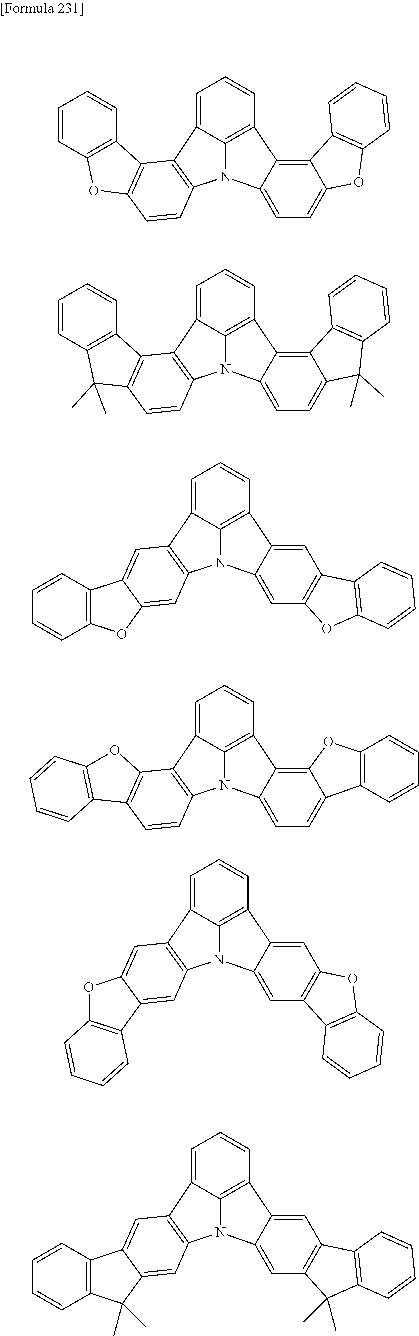 [SMILES: c1ccc2c(c1)oc1ccc3c(c4cccc5c6c7c(ccc6n3c45)oc3ccccc37)c12 ; CC1(C)c2ccccc2-c2c1ccc1c2c2cccc3c4c5c(ccc4n1c23)C(C)(C)c1ccccc1-5 ; c1ccc2c(c1)oc1cc3c(cc12)c1cccc2c4cc5c(cc4n3c12)oc1ccccc15 ; c1ccc2c(c1)oc1c2ccc2c1c1cccc3c4c5oc6ccccc6c5ccc4n2c13 ; c1ccc2c(c1)oc1cc3c4cccc5c6cc7oc8ccccc8c7cc6n(c3cc12)c45 ; CC1(C)c2ccccc2-c2cc3c4cccc5c6cc7c(cc6n(c3cc21)c45)C(C)(C)c1ccccc1-7]

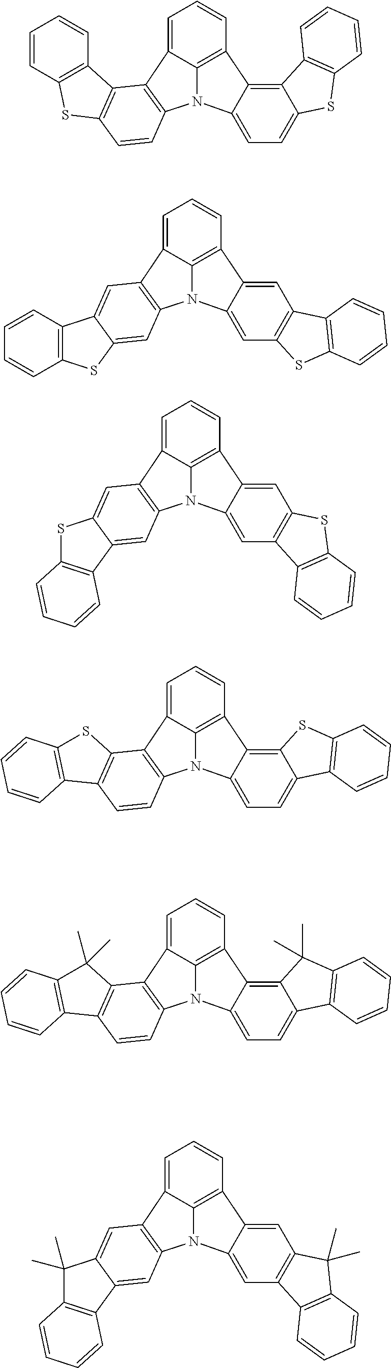

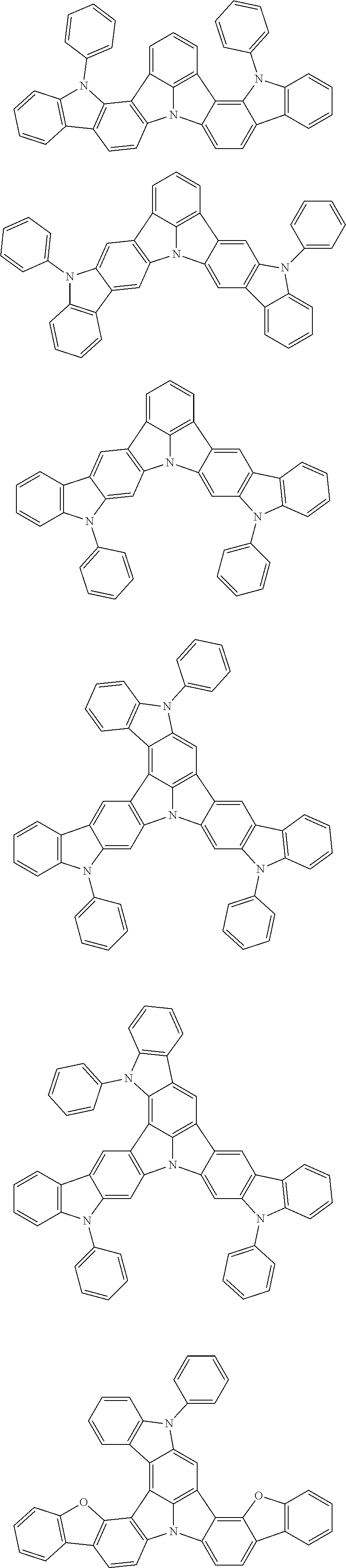

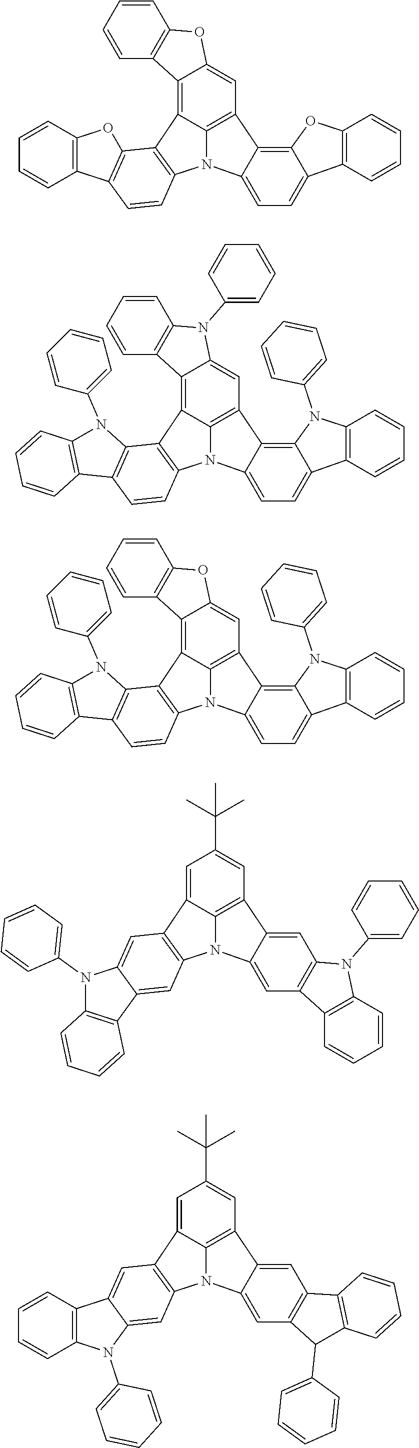

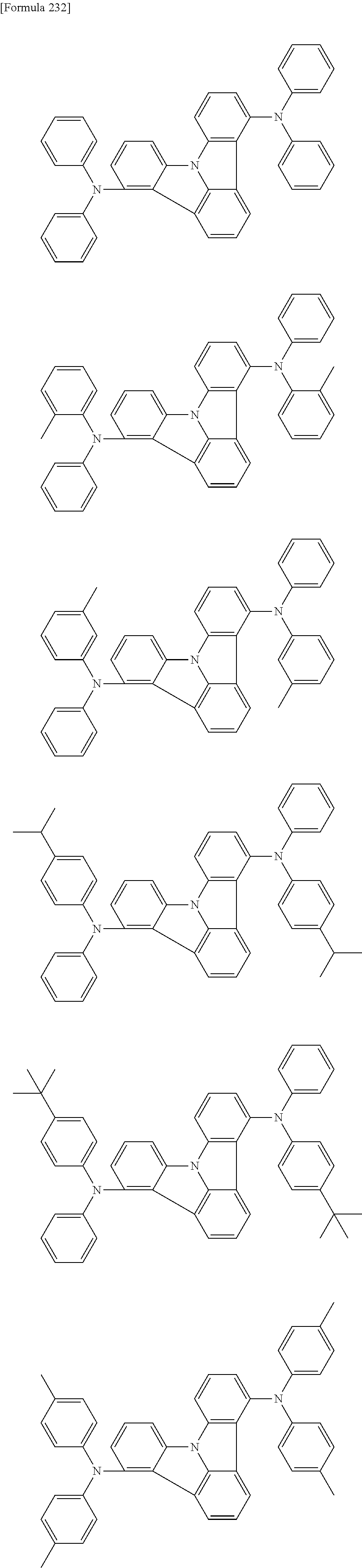

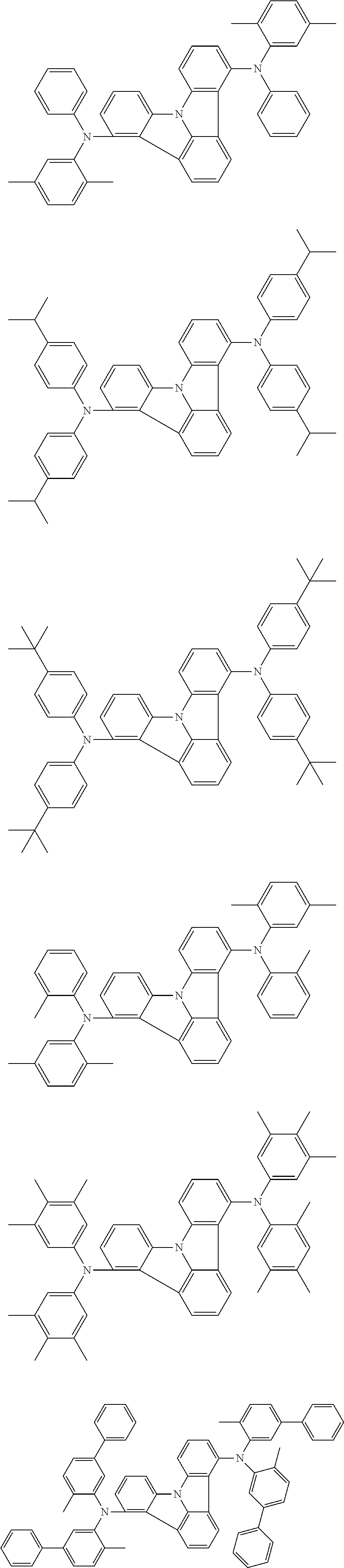

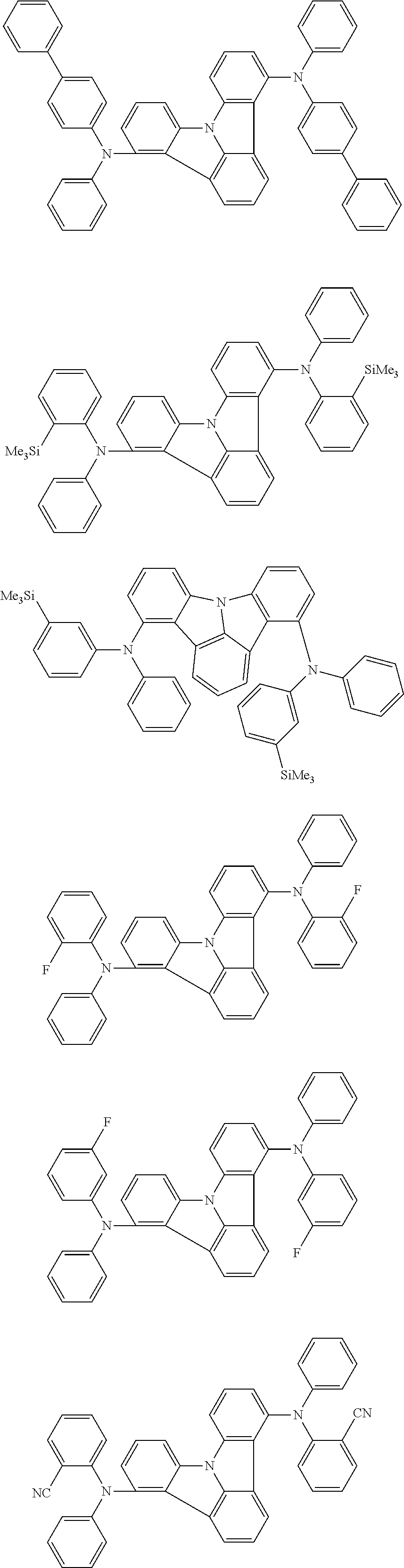

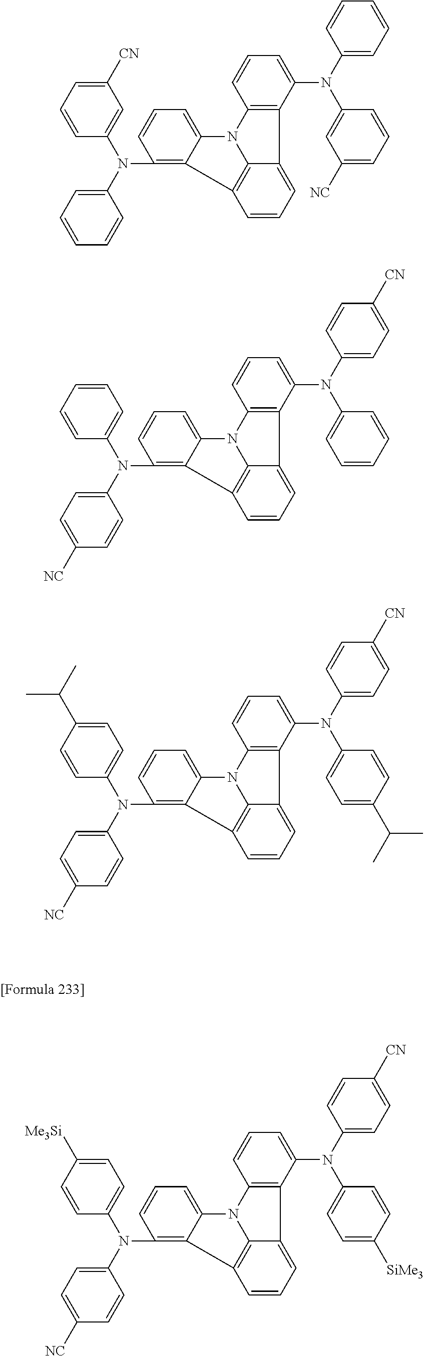 [SMILES: N#Cc1cccc(N(c2ccccc2)c2cccc3c2c2cccc4c5c(N(c6ccccc6)c6cccc(C#N)c6)cccc5n3c24)c1 ; N#Cc1ccc(N(c2ccccc2)c2cccc3c2c2cccc4c5c(N(c6ccccc6)c6ccc(C#N)cc6)cccc5n3c24)cc1 ; CC(C)c1ccc(N(c2ccc(C#N)cc2)c2cccc3c2c2cccc4c5c(N(c6ccc(C#N)cc6)c6ccc(C(C)C)cc6)cccc5n3c24)cc1 ; C[Si](C)(C)c1ccc(N(c2ccc(C#N)cc2)c2cccc3c2c2cccc4c5c(N(c6ccc(C#N)cc6)c6ccc([Si](C)(C)C)cc6)cccc5n3c24)cc1]

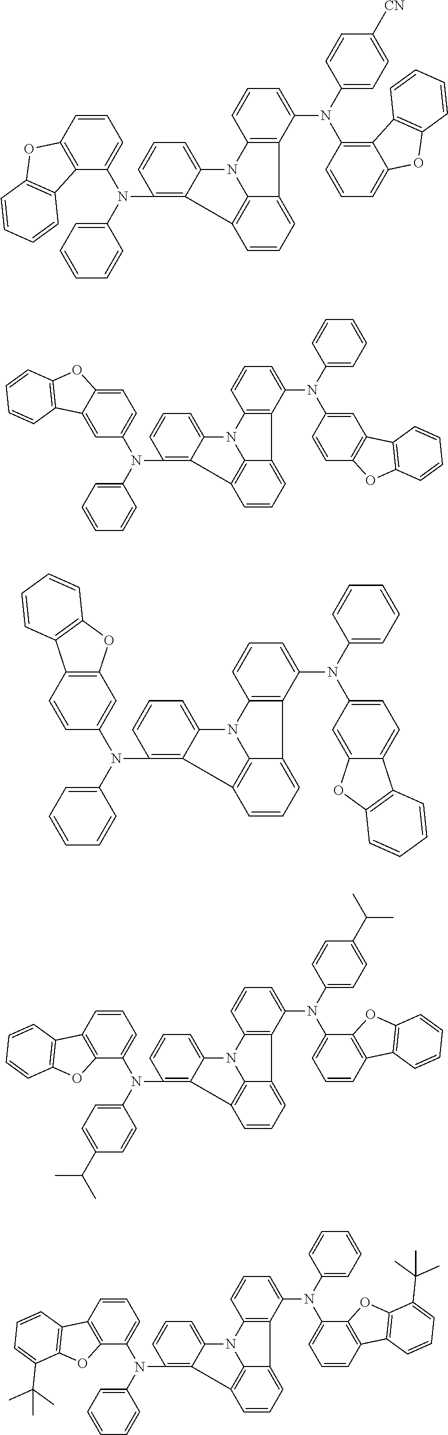

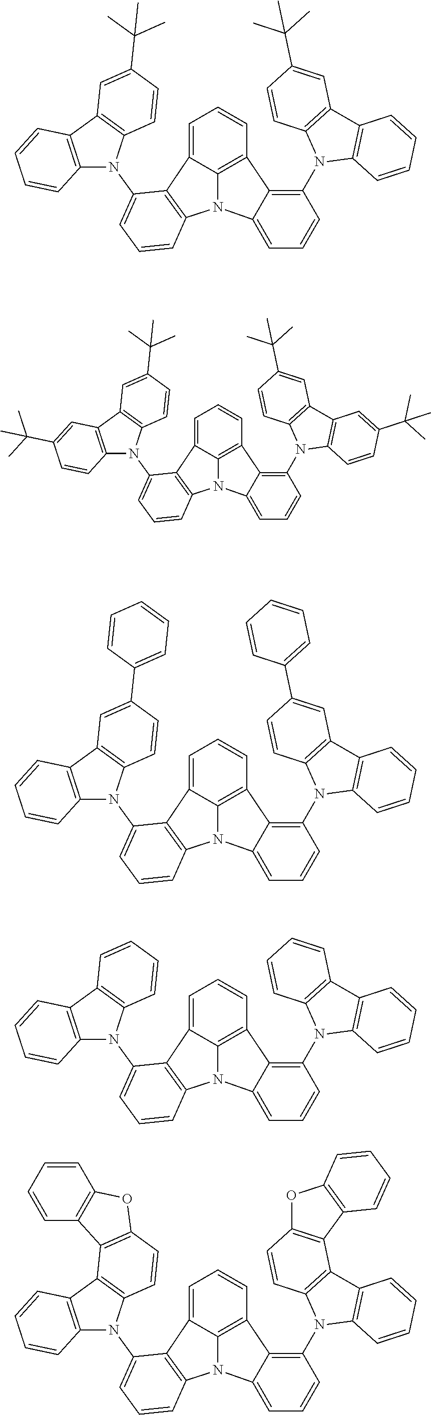 [SMILES: CC(C)(C)c1ccc2c(c1)c1ccccc1n2-c1cccc2c1c1cccc3c4c(-n5c6ccccc6c6cc(C(C)(C)C)ccc65)cccc4n2c13 ; CC(C)(C)c1ccc2c(c1)c1cc(C(C)(C)C)ccc1n2-c1cccc2c1c1cccc3c4c(-n5c6ccc(C(C)(C)C)cc6c6cc(C(C)(C)C)ccc65)cccc4n2c13 ; c1ccc(-c2ccc3c(c2)c2ccccc2n3-c2cccc3c2c2cccc4c5c(-n6c7ccccc7c7cc(-c8ccccc8)ccc76)cccc5n3c24)cc1 ; c1ccc2c(c1)c1ccccc1n2-c1cccc2c1c1cccc3c4c(-n5c6ccccc6c6ccccc65)cccc4n2c13 ; c1ccc2c(c1)oc1ccc3c(c4ccccc4n3-c3cccc4c3c3cccc5c6c(-n7c8ccccc8c8c9c(ccc87)oc7ccccc79)cccc6n4c35)c12]

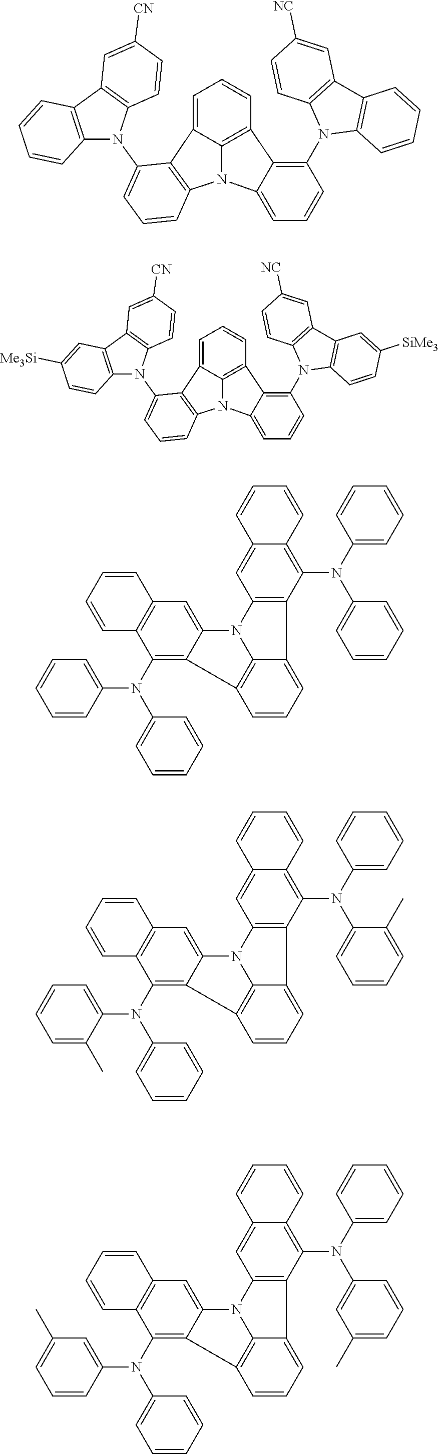 [SMILES: N#Cc1ccc2c(c1)c1ccccc1n2-c1cccc2c1c1cccc3c4c(-n5c6ccccc6c6cc(C#N)ccc65)cccc4n2c13 ; C[Si](C)(C)c1ccc2c(c1)c1cc(C#N)ccc1n2-c1cccc2c1c1cccc3c4c(-n5c6ccc(C#N)cc6c6cc([Si](C)(C)C)ccc65)cccc4n2c13 ; c1ccc(N(c2ccccc2)c2c3ccccc3cc3c2c2cccc4c5c(N(c6ccccc6)c6ccccc6)c6ccccc6cc5n3c24)cc1 ; Cc1ccccc1N(c1ccccc1)c1c2ccccc2cc2c1c1cccc3c4c(N(c5ccccc5)c5ccccc5C)c5ccccc5cc4n2c13 ; Cc1cccc(N(c2ccccc2)c2c3ccccc3cc3c2c2cccc4c5c(N(c6ccccc6)c6cccc(C)c6)c6ccccc6cc5n3c24)c1]

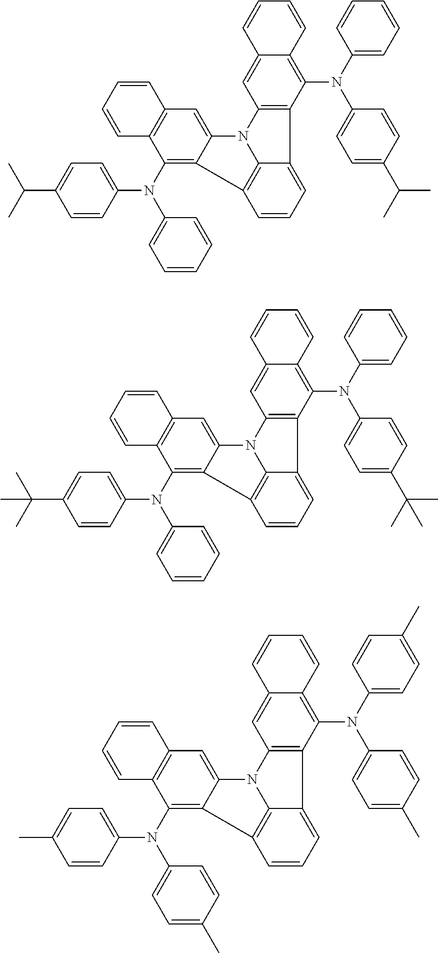

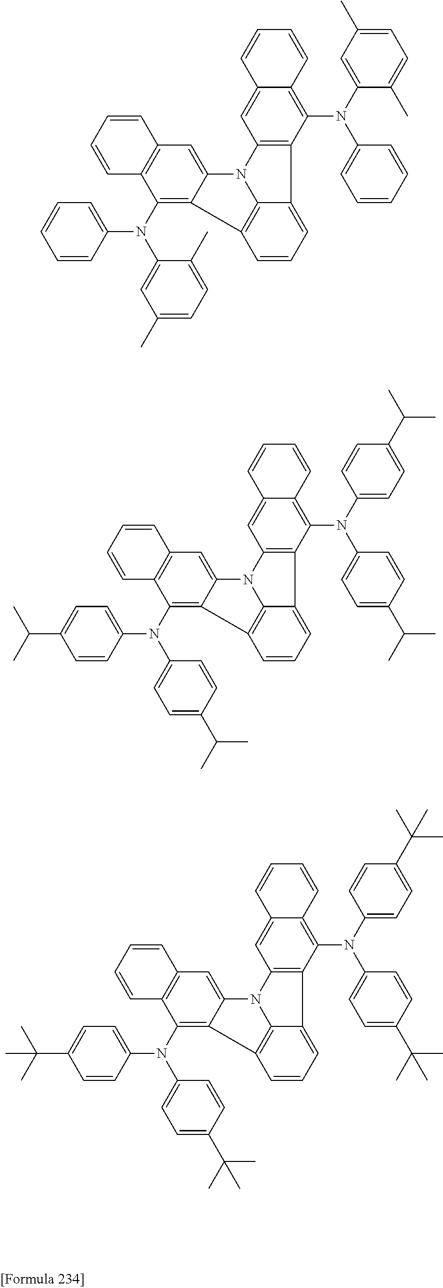

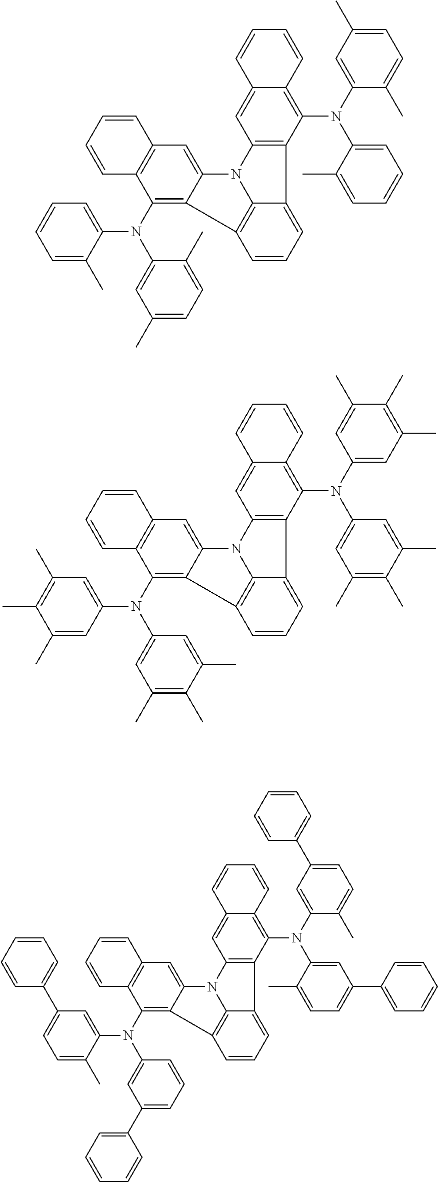

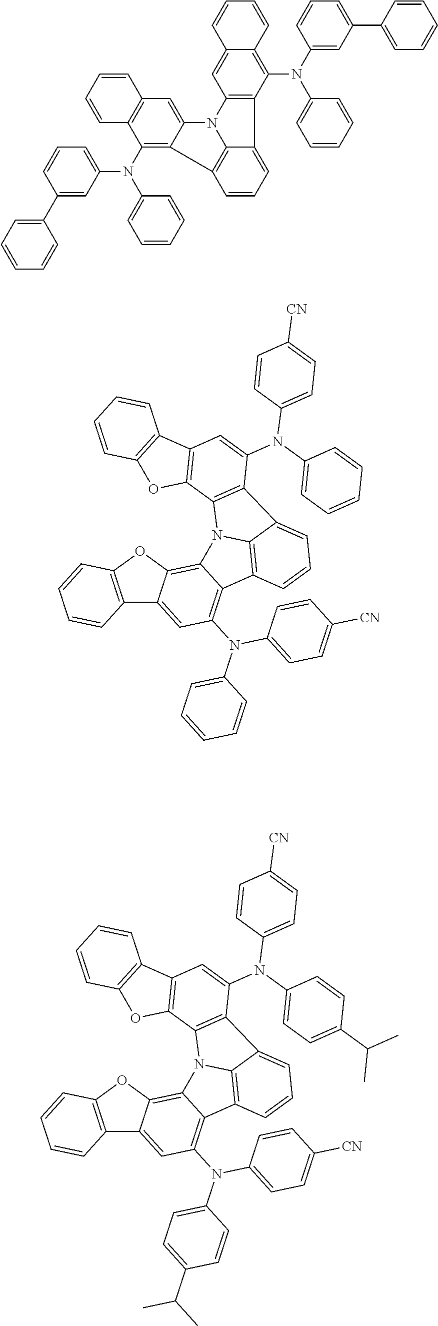 [SMILES: c1ccc(-c2cccc(N(c3ccccc3)c3c4ccccc4cc4c3c3cccc5c6c(N(c7ccccc7)c7cccc(-c8ccccc8)c7)c7ccccc7cc6n4c35)c2)cc1 ; N#Cc1ccc(N(c2ccccc2)c2cc3c4ccccc4oc3c3c2c2cccc4c5c(N(c6ccccc6)c6ccc(C#N)cc6)cc6c7ccccc7oc6c5n3c24)cc1 ; CC(C)c1ccc(N(c2ccc(C#N)cc2)c2cc3c4ccccc4oc3c3c2c2cccc4c5c(N(c6ccc(C#N)cc6)c6ccc(C(C)C)cc6)cc6c7ccccc7oc6c5n3c24)cc1]

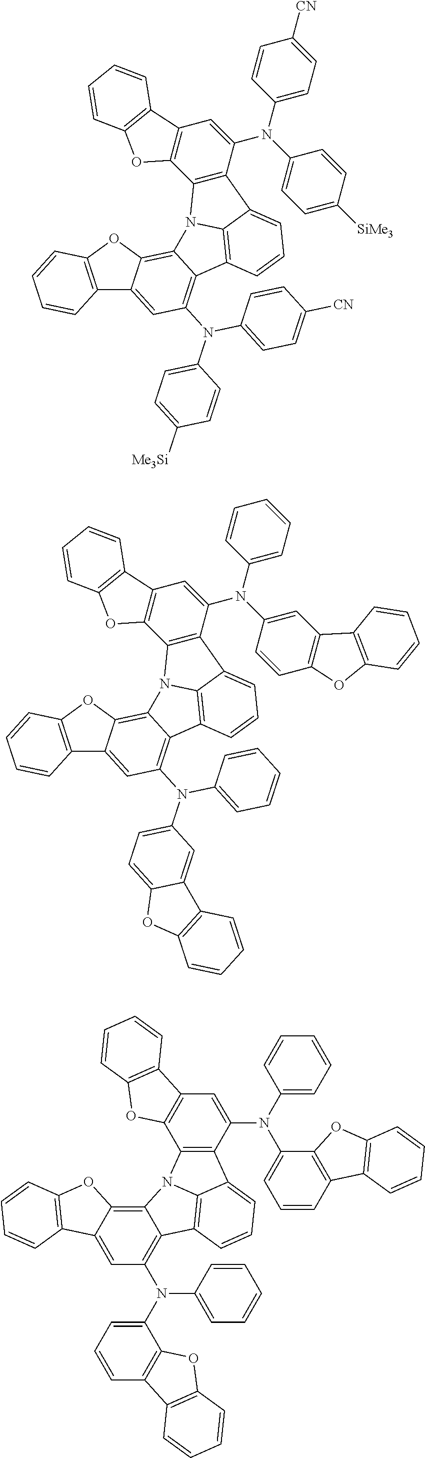 [SMILES: C[Si](C)(C)c1ccc(N(c2ccc(C#N)cc2)c2cc3c4ccccc4oc3c3c2c2cccc4c5c(N(c6ccc(C#N)cc6)c6ccc([Si](C)(C)C)cc6)cc6c7ccccc7oc6c5n3c24)cc1 ; c1ccc(N(c2ccc3oc4ccccc4c3c2)c2cc3c4ccccc4oc3c3c2c2cccc4c5c(N(c6ccccc6)c6ccc7oc8ccccc8c7c6)cc6c7ccccc7oc6c5n3c24)cc1 ; c1ccc(N(c2cccc3c2oc2ccccc23)c2cc3c4ccccc4oc3c3c2c2cccc4c5c(N(c6ccccc6)c6cccc7c6oc6ccccc67)cc6c7ccccc7oc6c5n3c24)cc1]

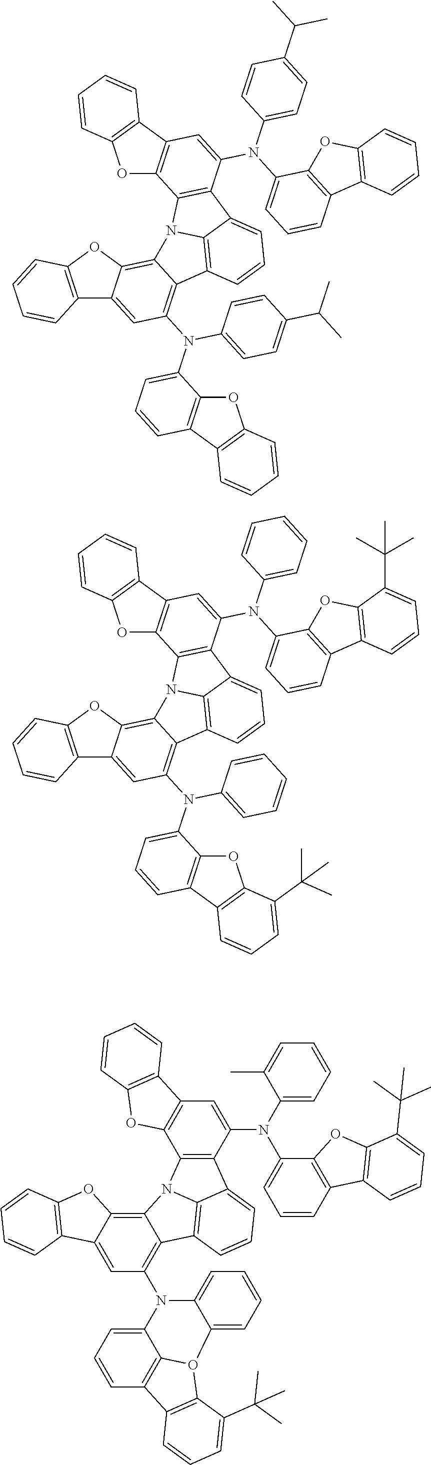

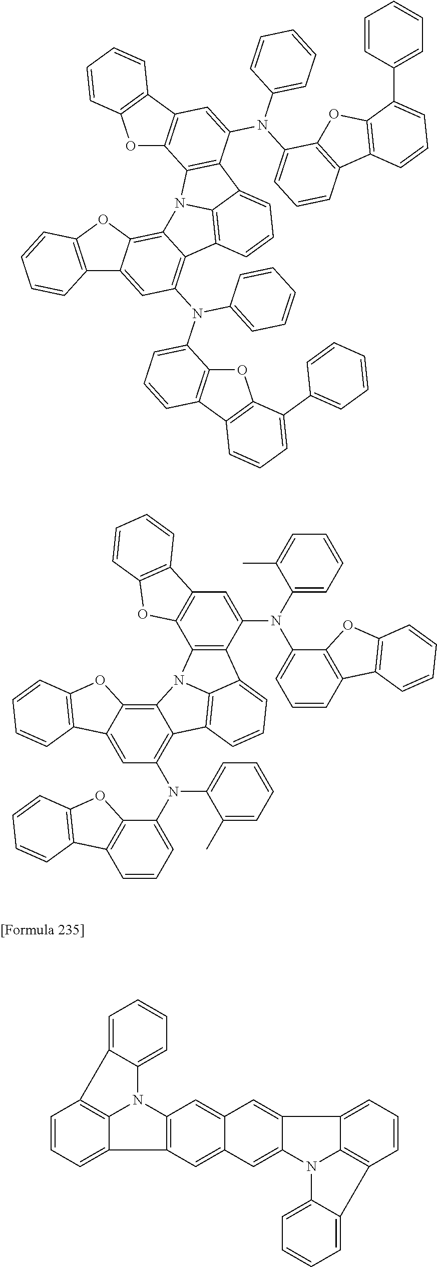 [SMILES: c1ccc(-c2cccc3c2oc2c(N(c4ccccc4)c4cc5c6ccccc6oc5c5c4c4cccc6c7c(N(c8ccccc8)c8cccc9c8oc8c(-c%10ccccc%10)cccc89)cc8c9ccccc9oc8c7n5c46)cccc23)cc1 ; Cc1ccccc1N(c1cccc2c1oc1ccccc12)c1cc2c3ccccc3oc2c2c1c1cccc3c4c(N(c5ccccc5C)c5cccc6c5oc5ccccc56)cc5c6ccccc6oc5c4n2c13 ; c1ccc2c(c1)c1cccc3c4cc5cc6c(cc5cc4n2c13)c1cccc2c3ccccc3n6c21]

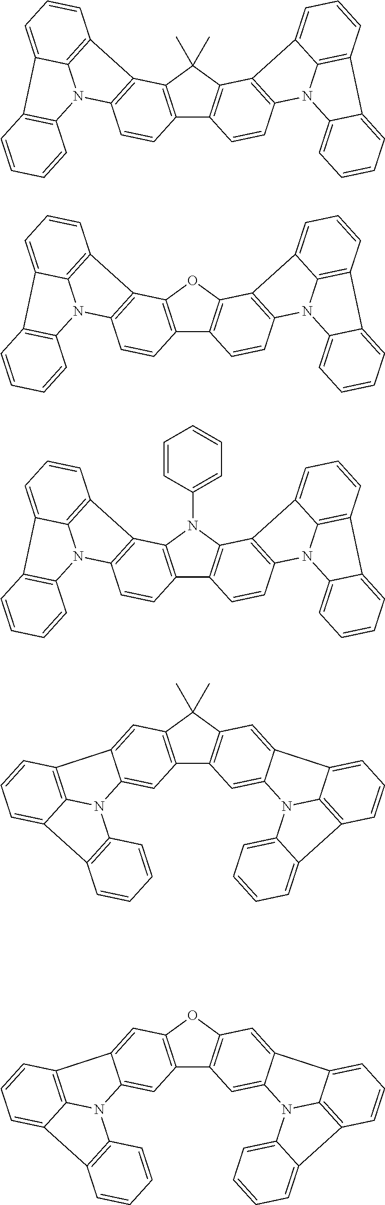

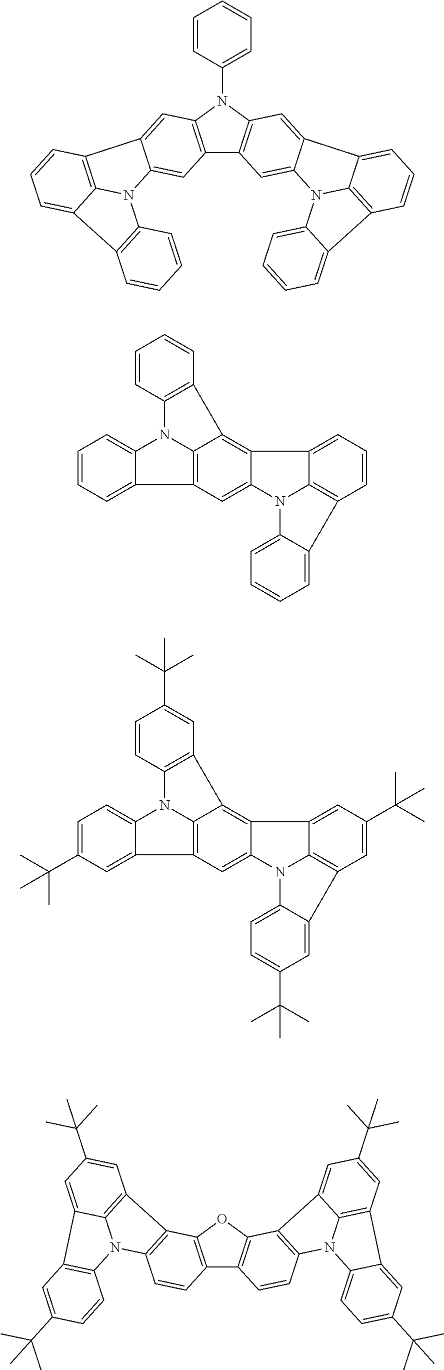

### Compound Represented by Formula (5)

The compound represented by the formula (5) will be described below. The compound represented by the formula (5) corresponds to the compound represented by the above-described formula (41-3).

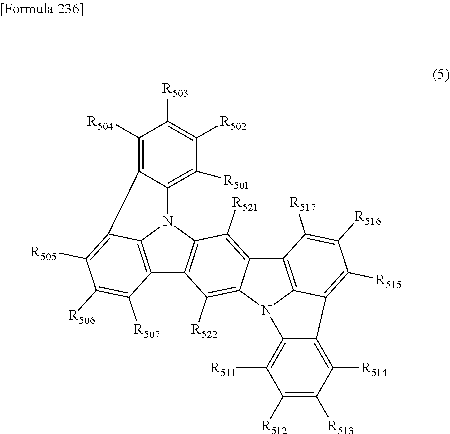

In the formula (5): at least one combination of adjacent two or more of R₅₀₁ to R₅₀₇ and R₅₁₁ to R₅₁₇ are mutually bonded to form a substituted or unsubstituted monocyclic ring, mutually bonded to form a substituted or unsubstituted fused ring, or not mutually bonded;
R₅₀₁ to R₅₀₇ and R₅₁₁ to R₅₁₇ neither forming the monocyclic ring nor forming the fused ring each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
R₅₂₁ and R₅₂₂ each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

“A combination of adjacent two or more of R₅₀₁ to R₅₀₇ and R₅₁₁ to R₅₁₇” refers to, for instance, a combination of R₅₀₁ and R₅₀₂, a combination of R₅₀₂ and R₅₀₃, a combination of R₅₀₃ and R₅₀₄, a combination of R₅₀₅ and R₅₀₆, a combination of R₅₀₆ and R₅₀₇, and a combination of R₅₀₁, R₅₀₂, and R₅₀₃.

In an exemplary embodiment, at least one, preferably two of R₅₀₁ to R₅₀₇ and R₅₁₁ to R₅₁₇ are groups represented by —N(R₉₀₆)(R₉₀₇).

In an exemplary embodiment, R₅₀₁ to R₅₀₇ and R₅₁₁ to R₅₁₇ are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, the compound represented by the formula (5) is a compound represented by a formula (52).

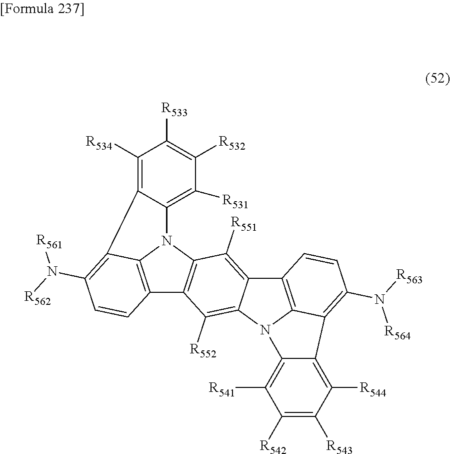

In the formula (52): at least one combination of adjacent two or more of R₅₃₁ to R₅₃₄ and R₅₄₁ to R₅₄₄ are mutually bonded to form a substituted or unsubstituted monocyclic ring, mutually bonded to form a substituted or unsubstituted fused ring, or not mutually bonded;
R₅₃₁ to R₅₃₄ and R₅₄₁ to R₅₄₄ neither forming the monocyclic ring nor forming the fused ring and R₅₅₁ and R₅₅₂ each independently represent a hydrogen atom, a substituted or unsubstituted aryl group having 6 to 50 ring carbon atoms, or a substituted or unsubstituted heterocyclic group having 5 to 50 ring atoms; and
R₅₆₁ to R₅₆₄ are each independently a substituted or unsubstituted aryl group having 6 to 50 ring carbon atoms, or a substituted or unsubstituted heterocyclic group having 5 to 50 ring atoms.

In an exemplary embodiment, the compound represented by the formula (5) is a compound represented by a formula (53).

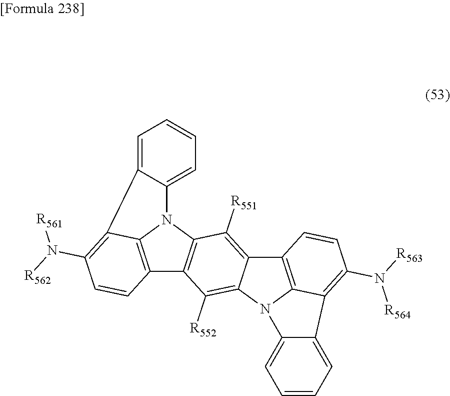

In the formula (53), R₅₅₁, R₅₅₂ and R₅₆₁ to R₅₆₄ each independently represent the same as R₅₅₁, R₅₅₂ and R₅₆₁ to R₅₆₄ in the formula (52).

In an exemplary embodiment, R₅₆₁ to R₅₆₄ in the formulae (52) and (53) are each independently a substituted or unsubstituted aryl group having 6 to 50 ring carbon atoms (preferably a phenyl group).

In an exemplary embodiment, R₅₂₁ and R₅₂₂ in the formula (5) and R₅₅₁ and R₅₅₂ in the formulae (52) and (53) are hydrogen atoms.

In an exemplary embodiment, the substituent for “substituted or unsubstituted” in the formulae (5), (52) and (53) is a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

### Specific Examples of Compound Represented by Formula (5)

Specific examples of the compound represented by the formula (5) include compounds shown below.

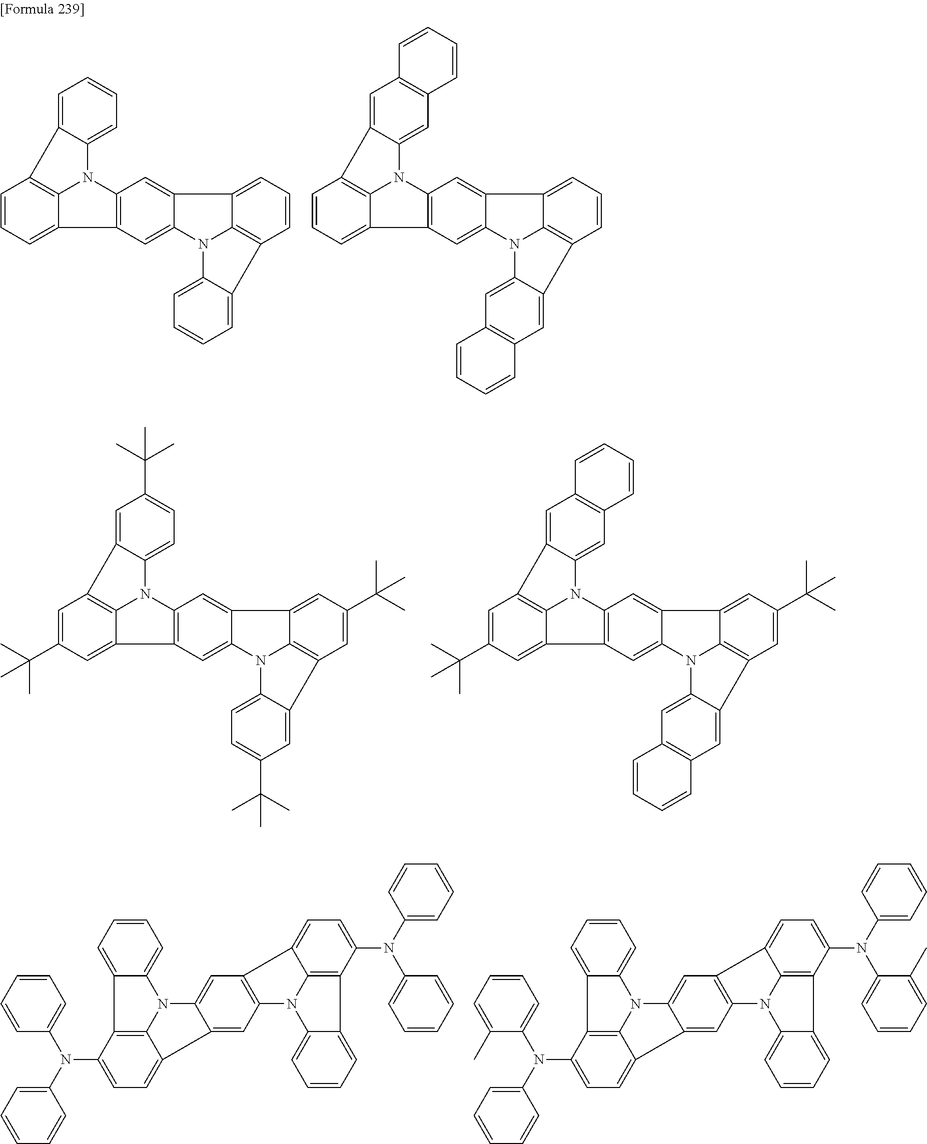

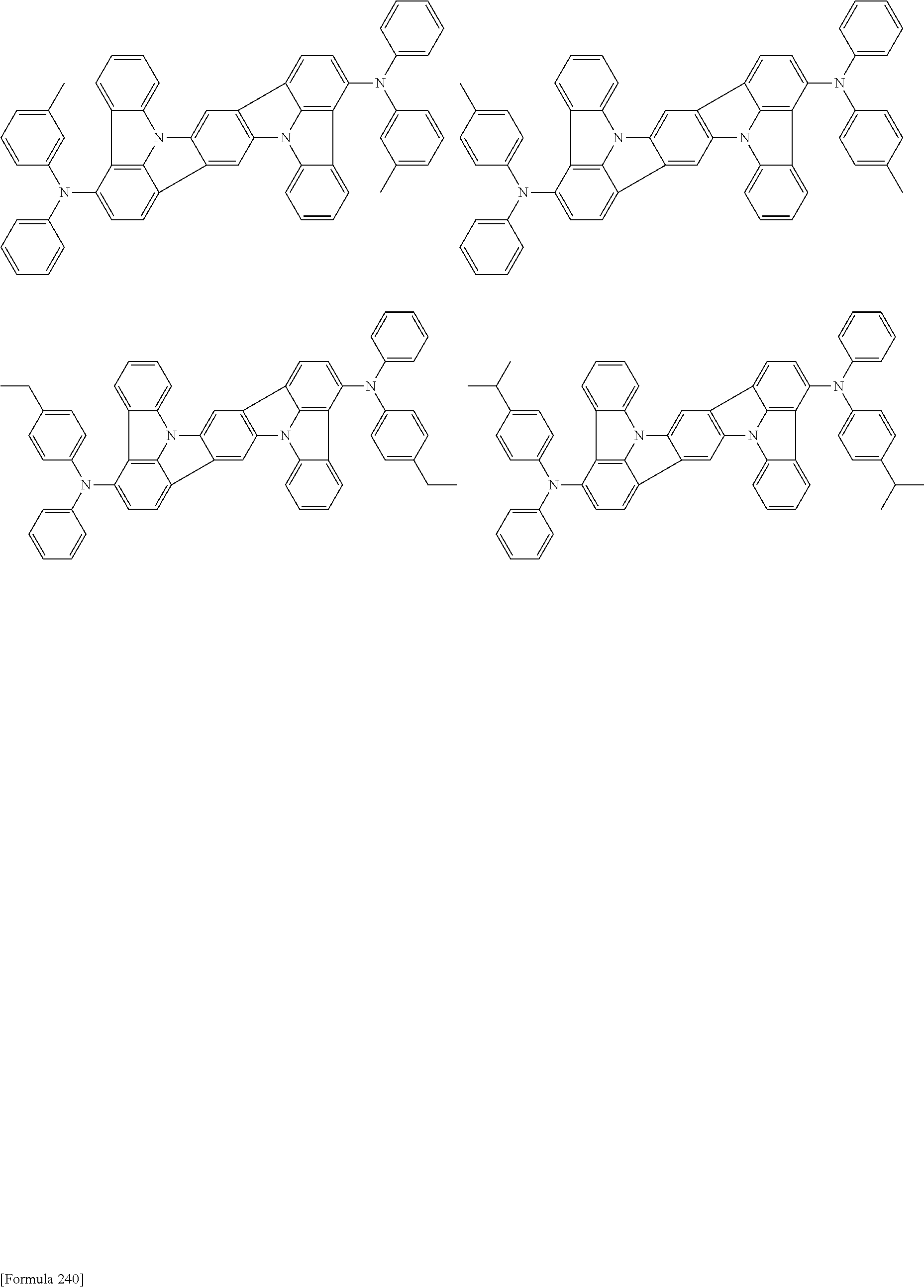

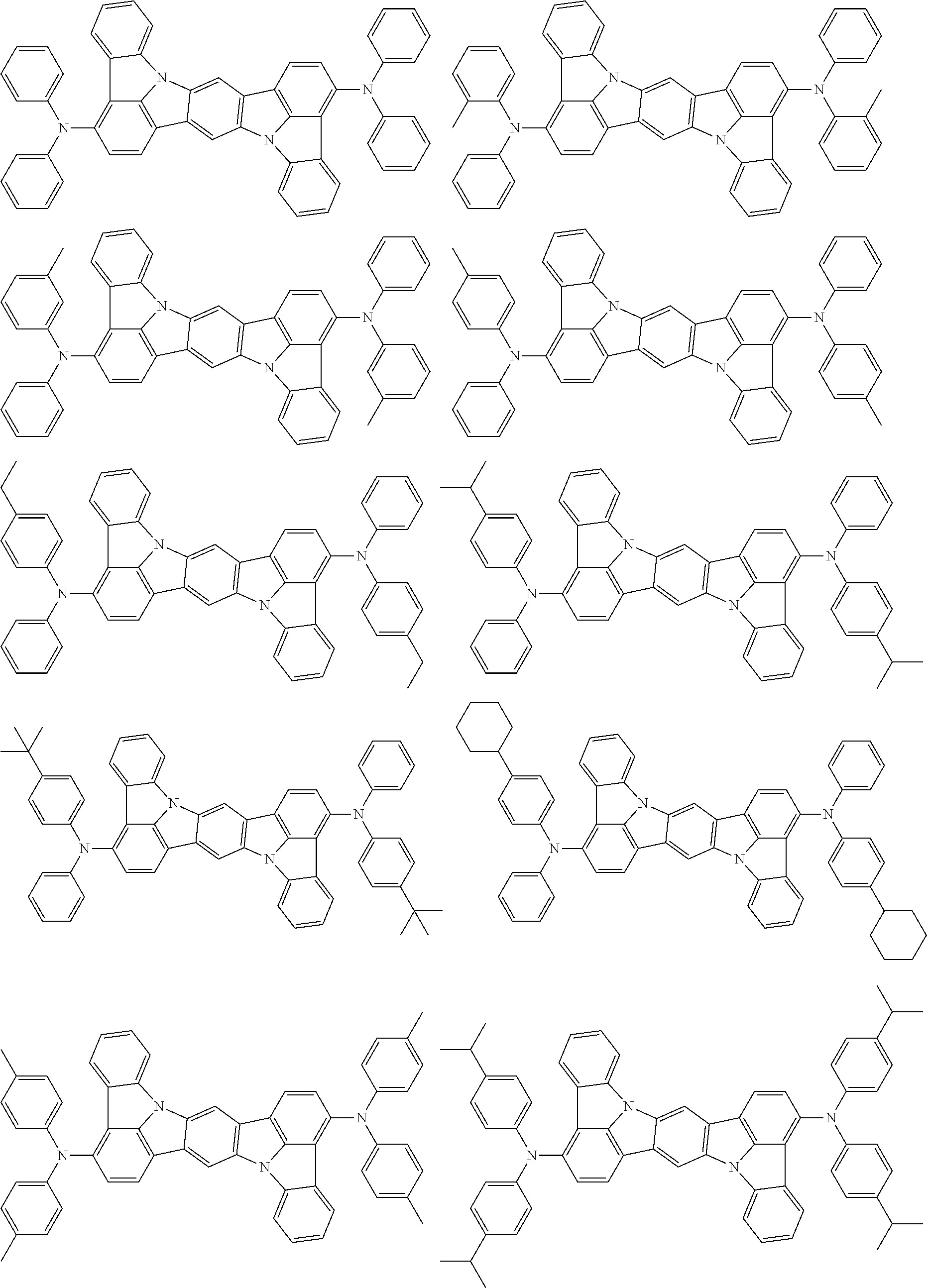

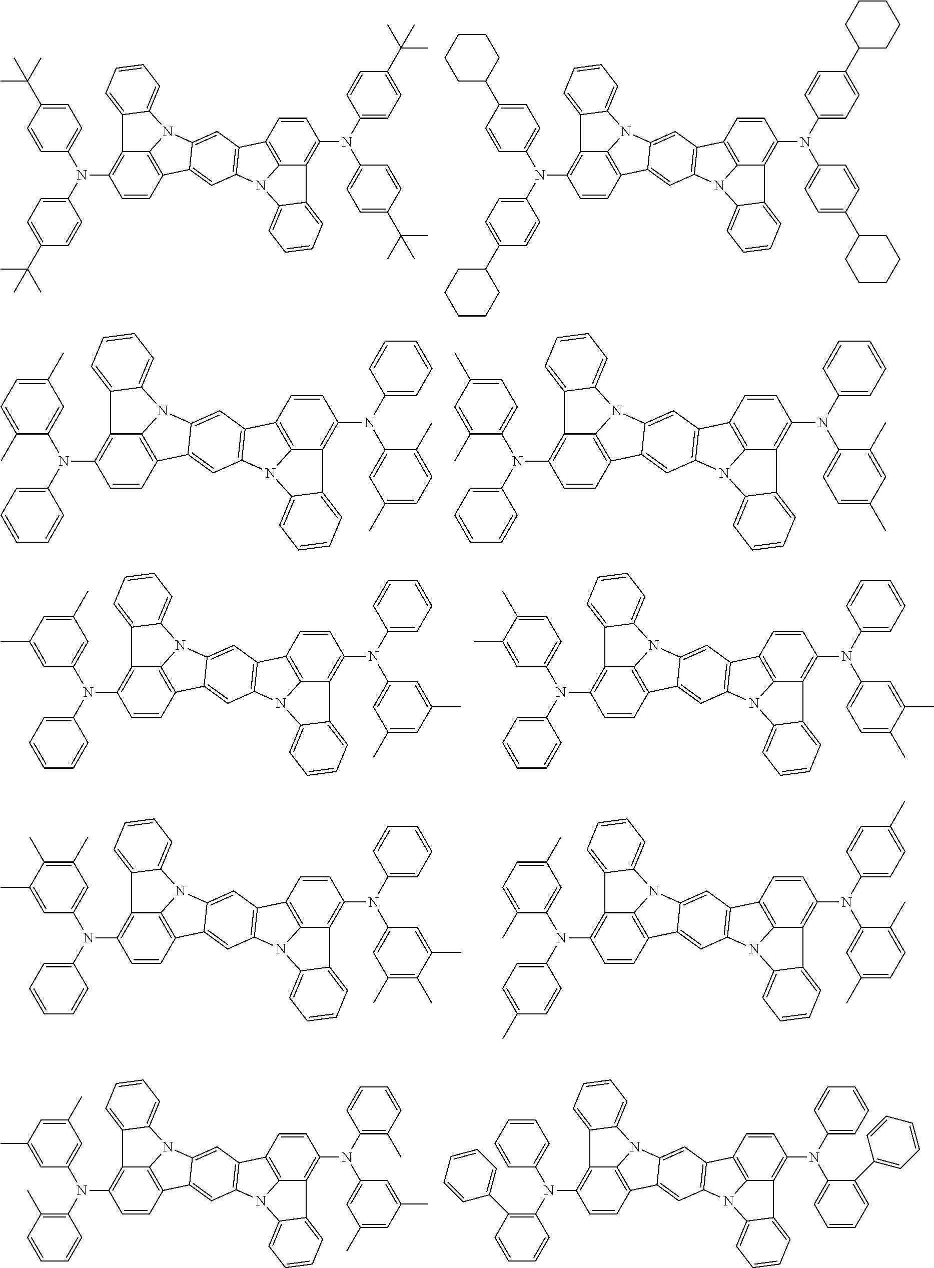

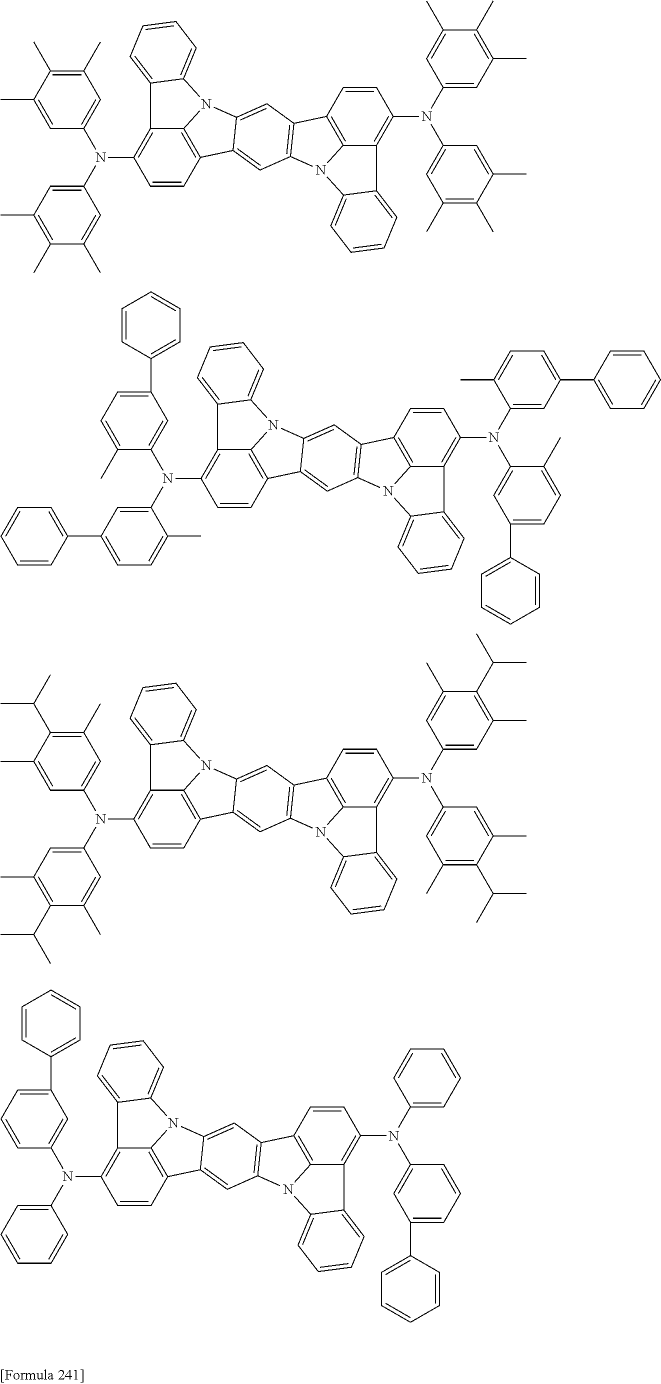

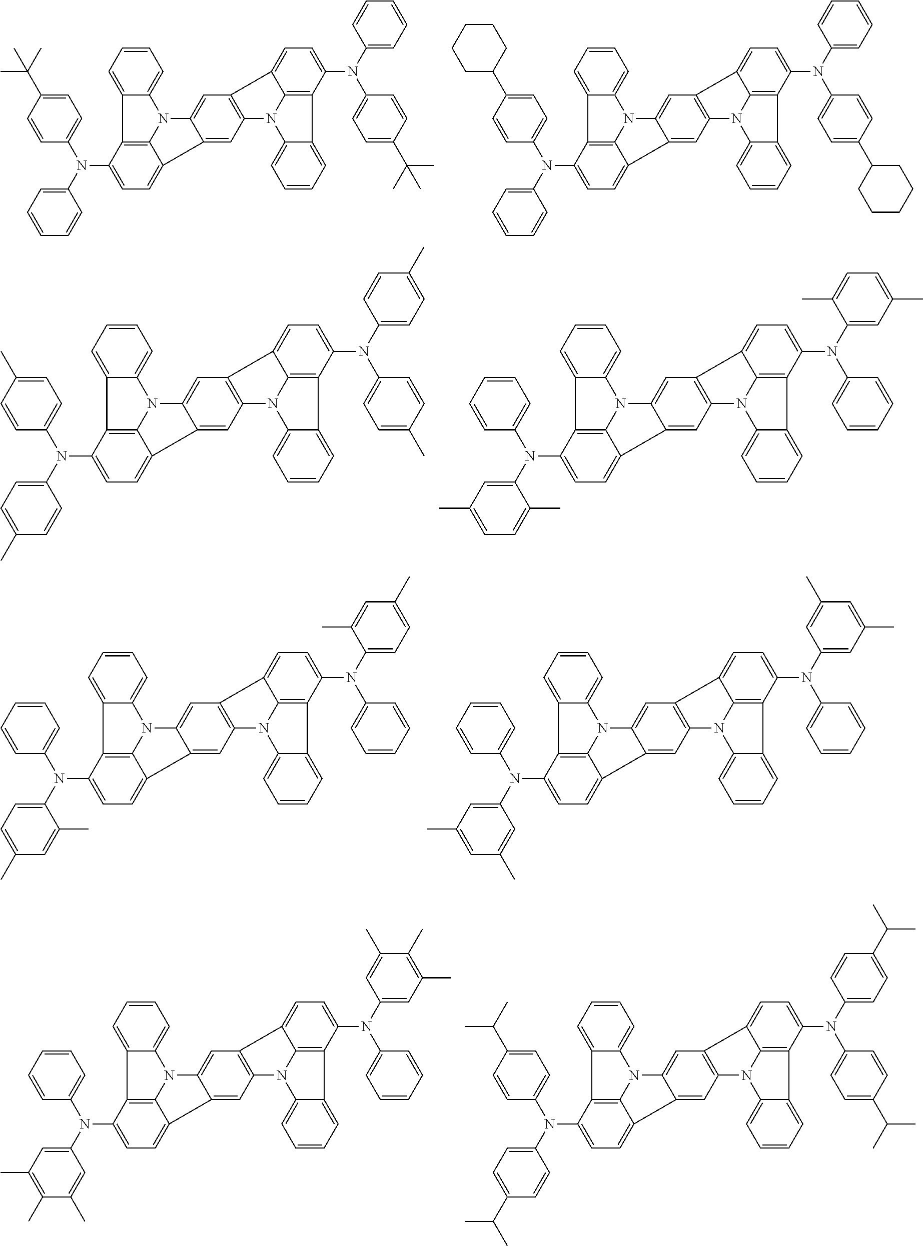

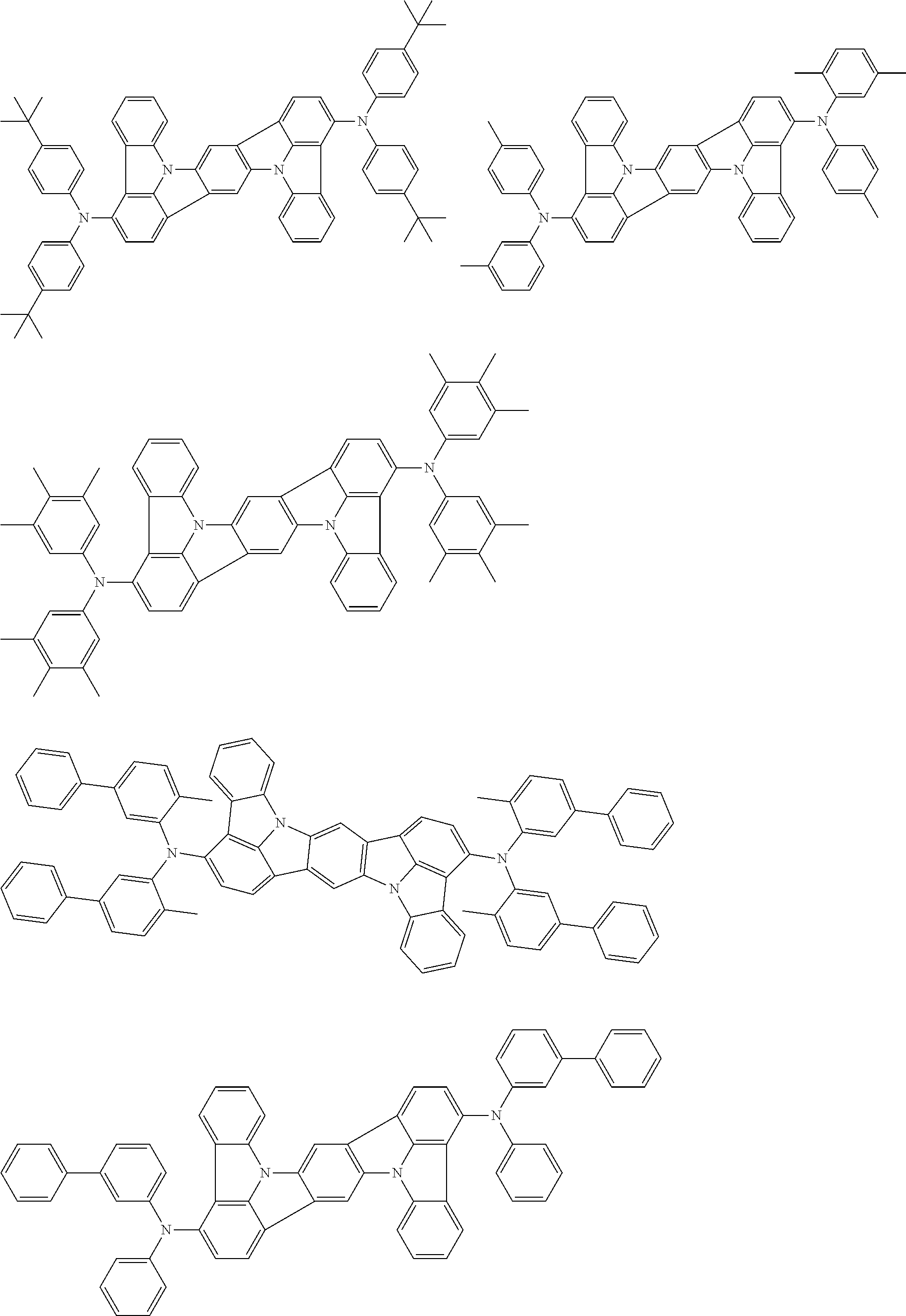

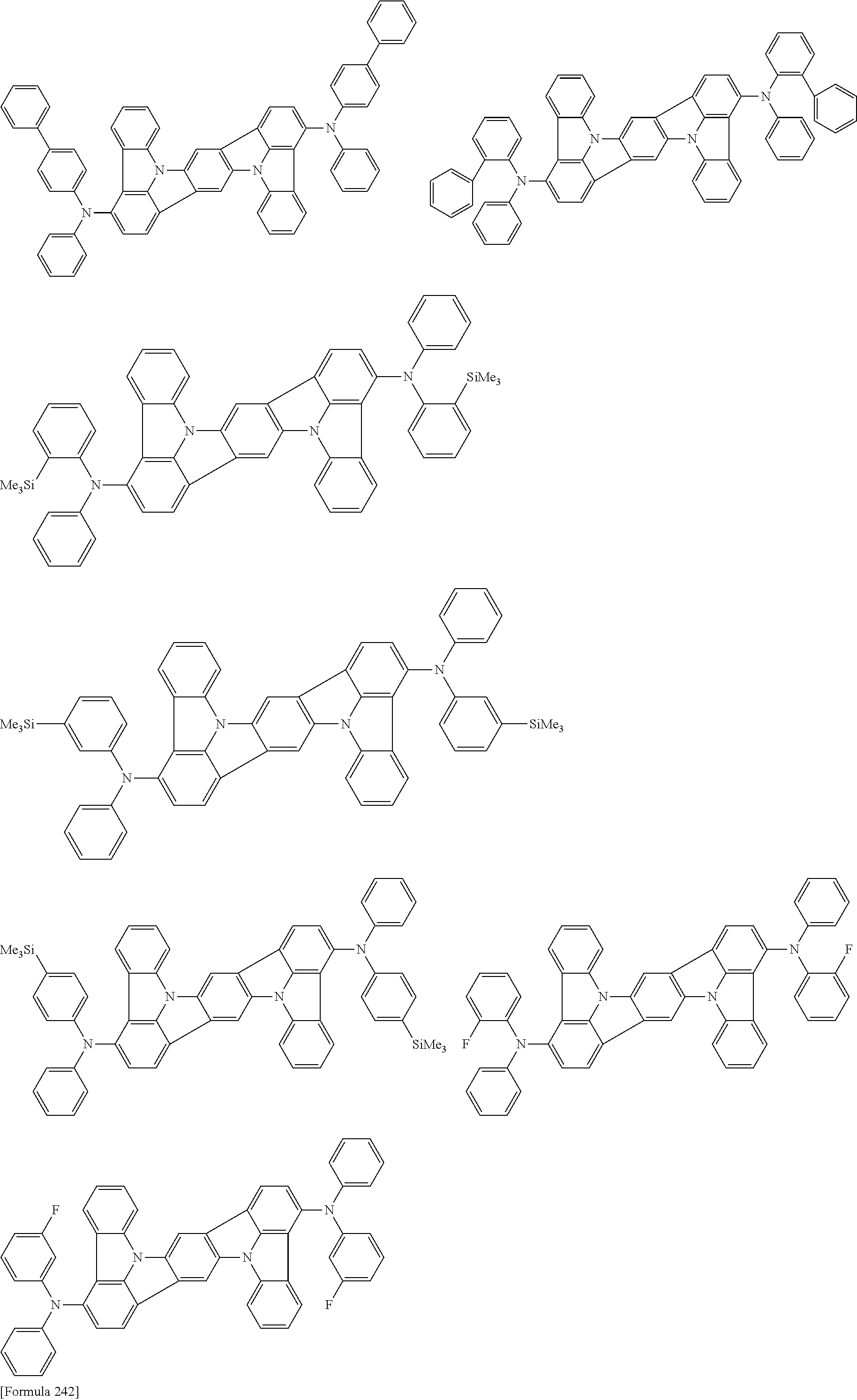

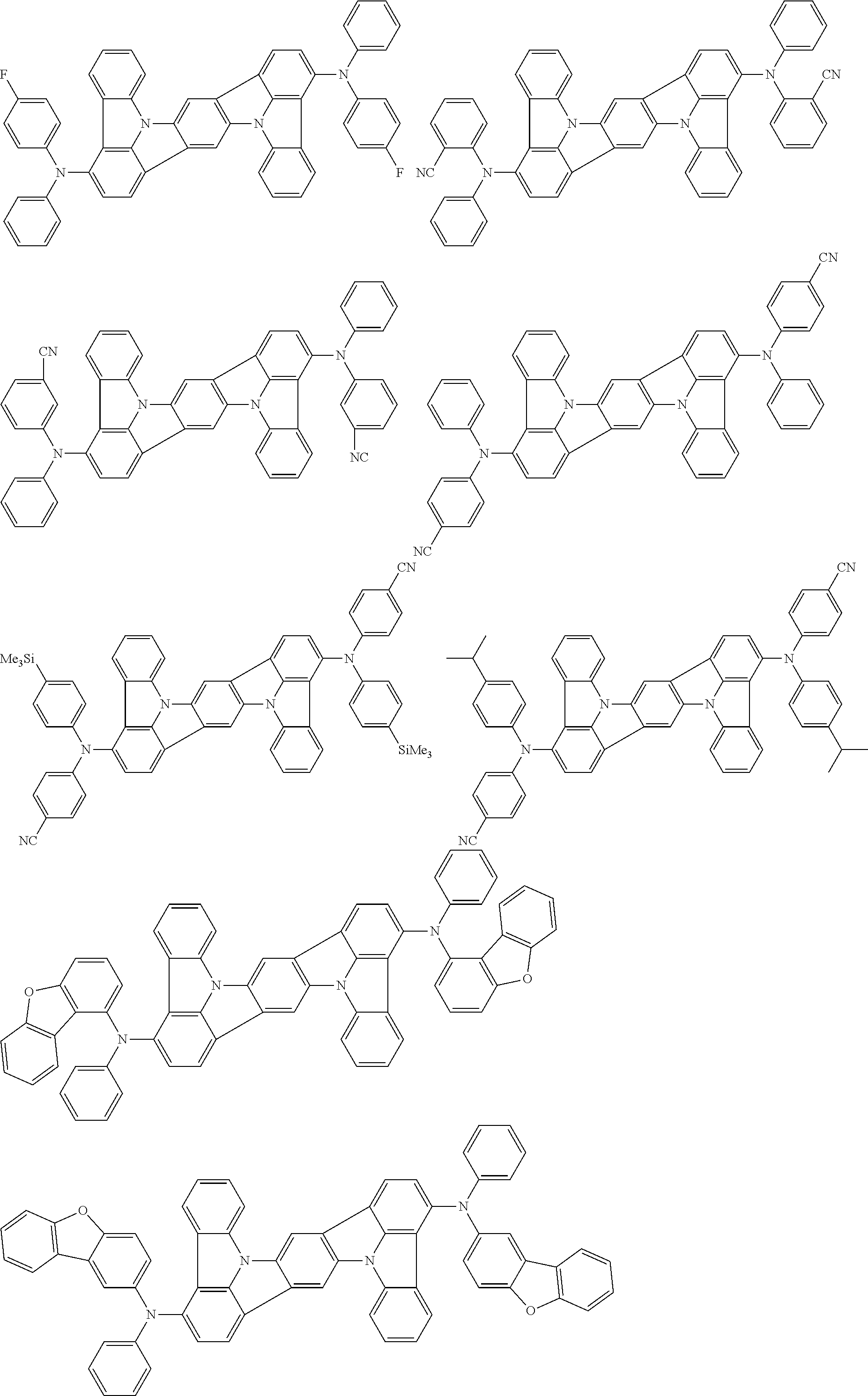

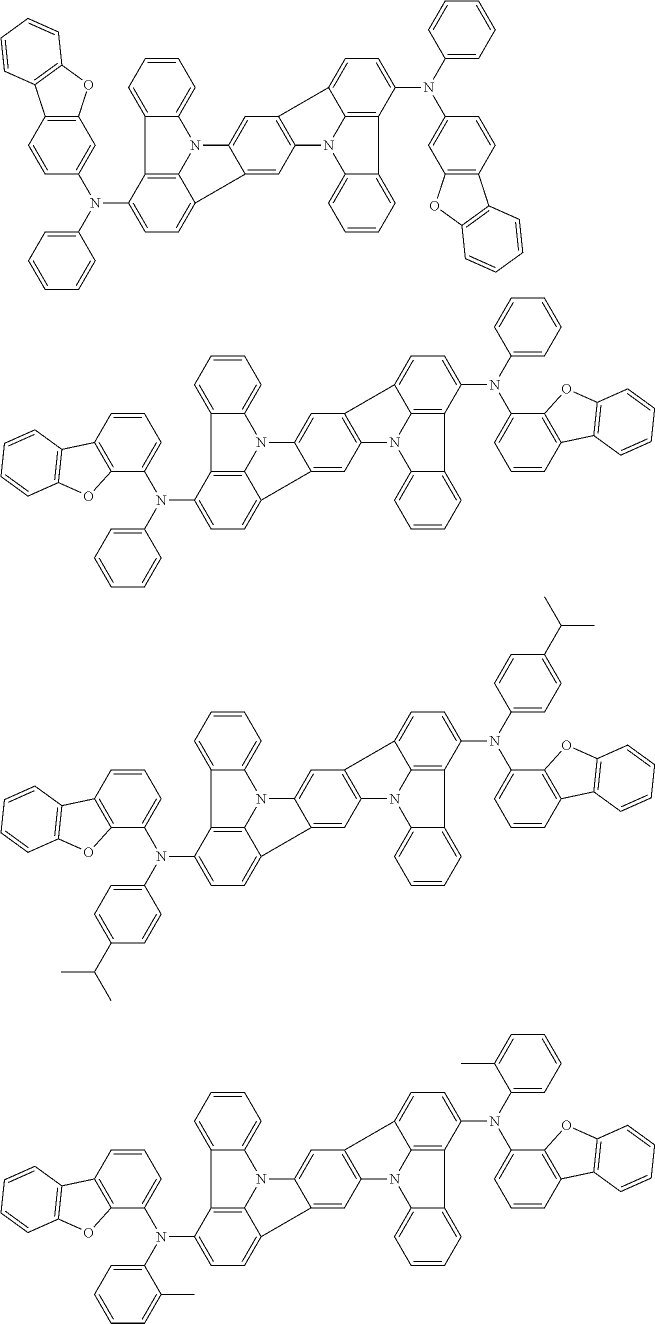

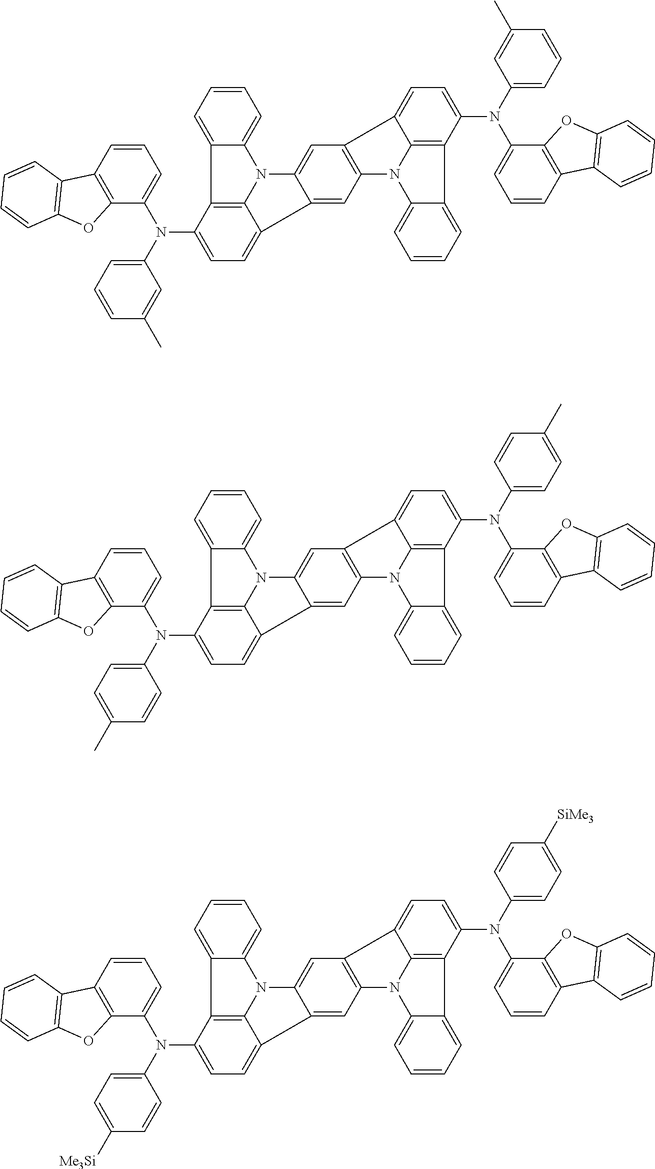 [SMILES: Cc1cccc(N(c2cccc3c2oc2ccccc23)c2ccc3c4cc5c(cc4n4c6ccccc6c2c34)c2ccc(N(c3cccc(C)c3)c3cccc4c3oc3ccccc34)c3c4ccccc4n5c23)c1 ; Cc1ccc(N(c2cccc3c2oc2ccccc23)c2ccc3c4cc5c(cc4n4c6ccccc6c2c34)c2ccc(N(c3ccc(C)cc3)c3cccc4c3oc3ccccc34)c3c4ccccc4n5c23)cc1 ; C[Si](C)(C)c1ccc(N(c2cccc3c2oc2ccccc23)c2ccc3c4cc5c(cc4n4c6ccccc6c2c34)c2ccc(N(c3ccc([Si](C)(C)C)cc3)c3cccc4c3oc3ccccc34)c3c4ccccc4n5c23)cc1]

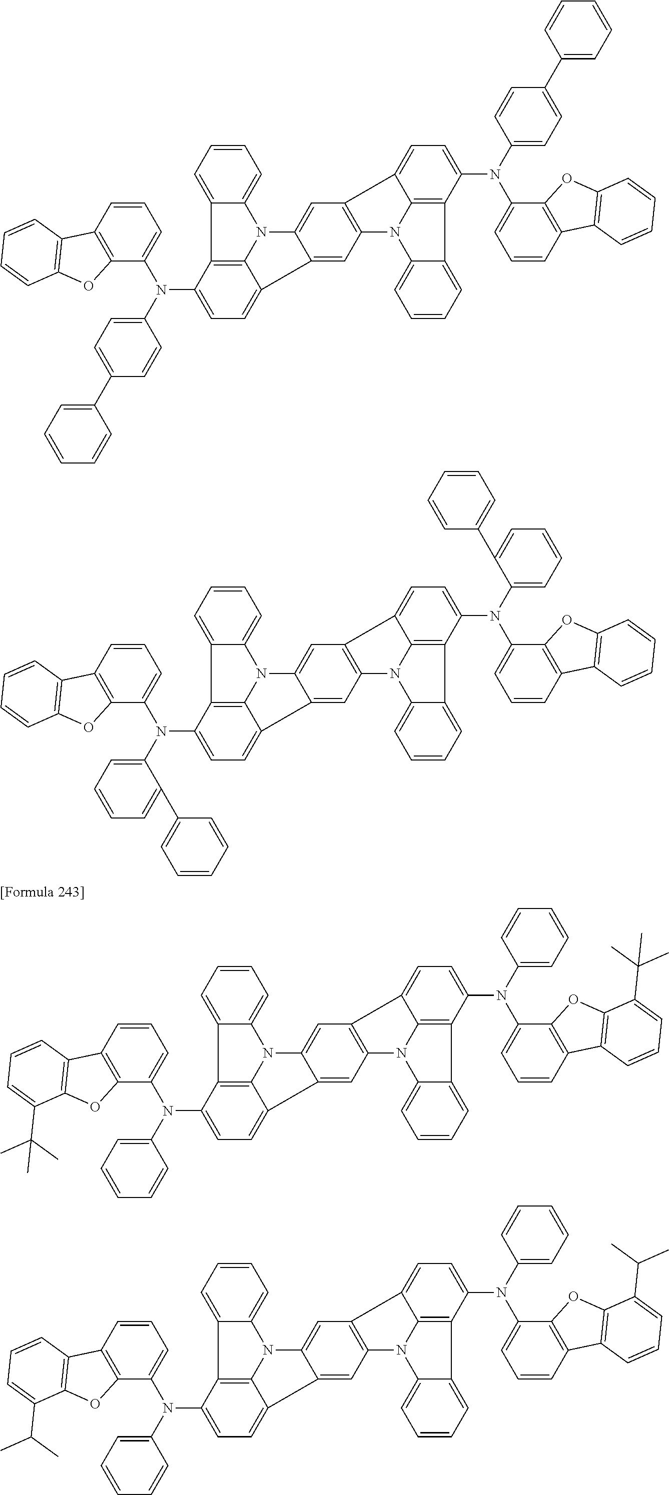

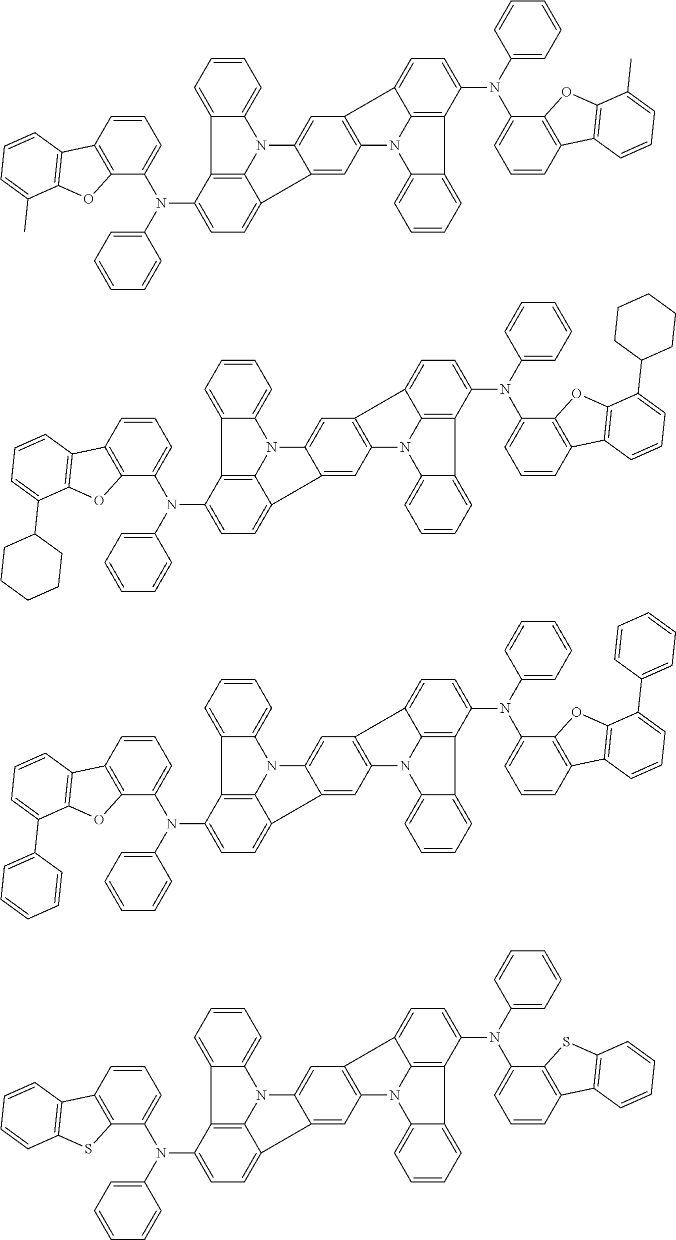

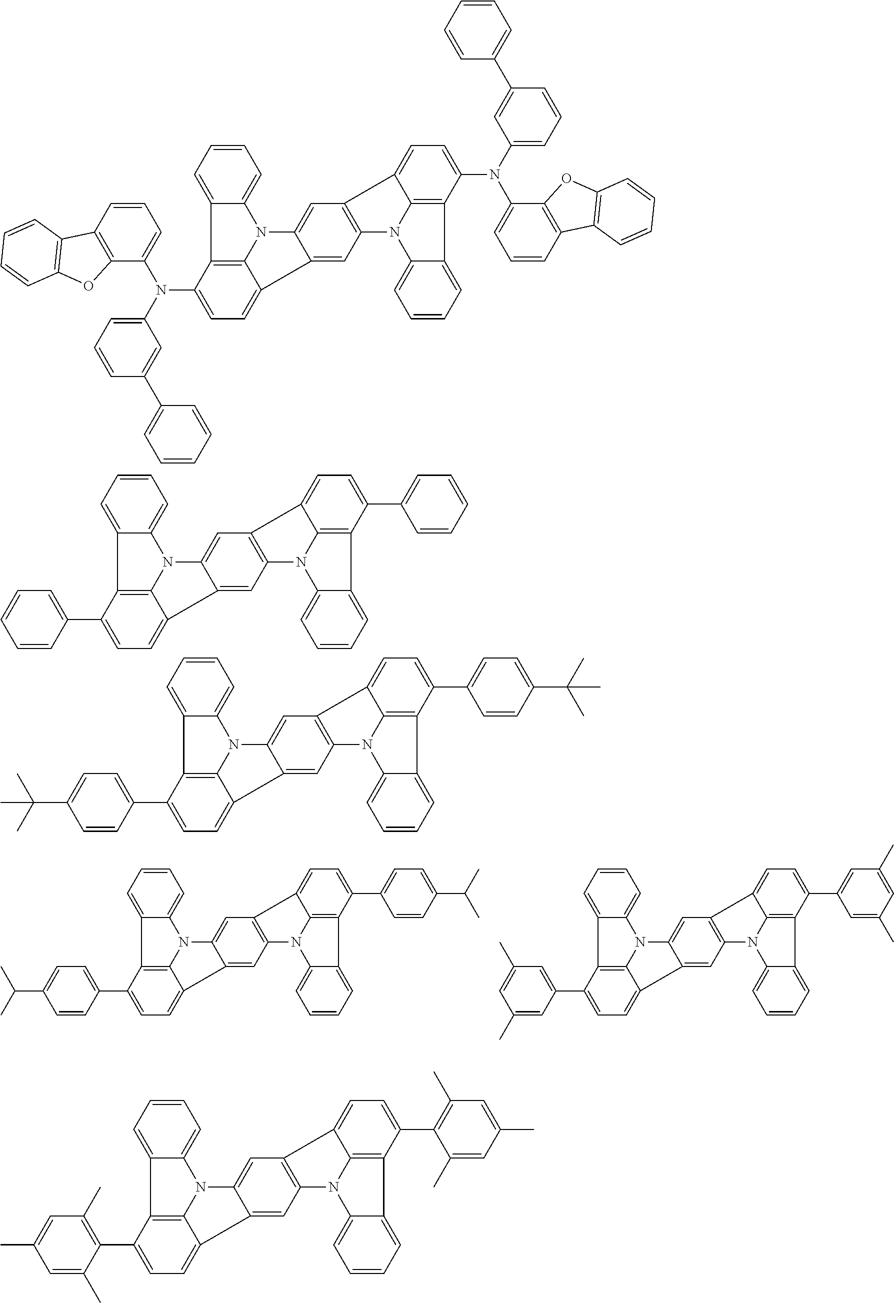

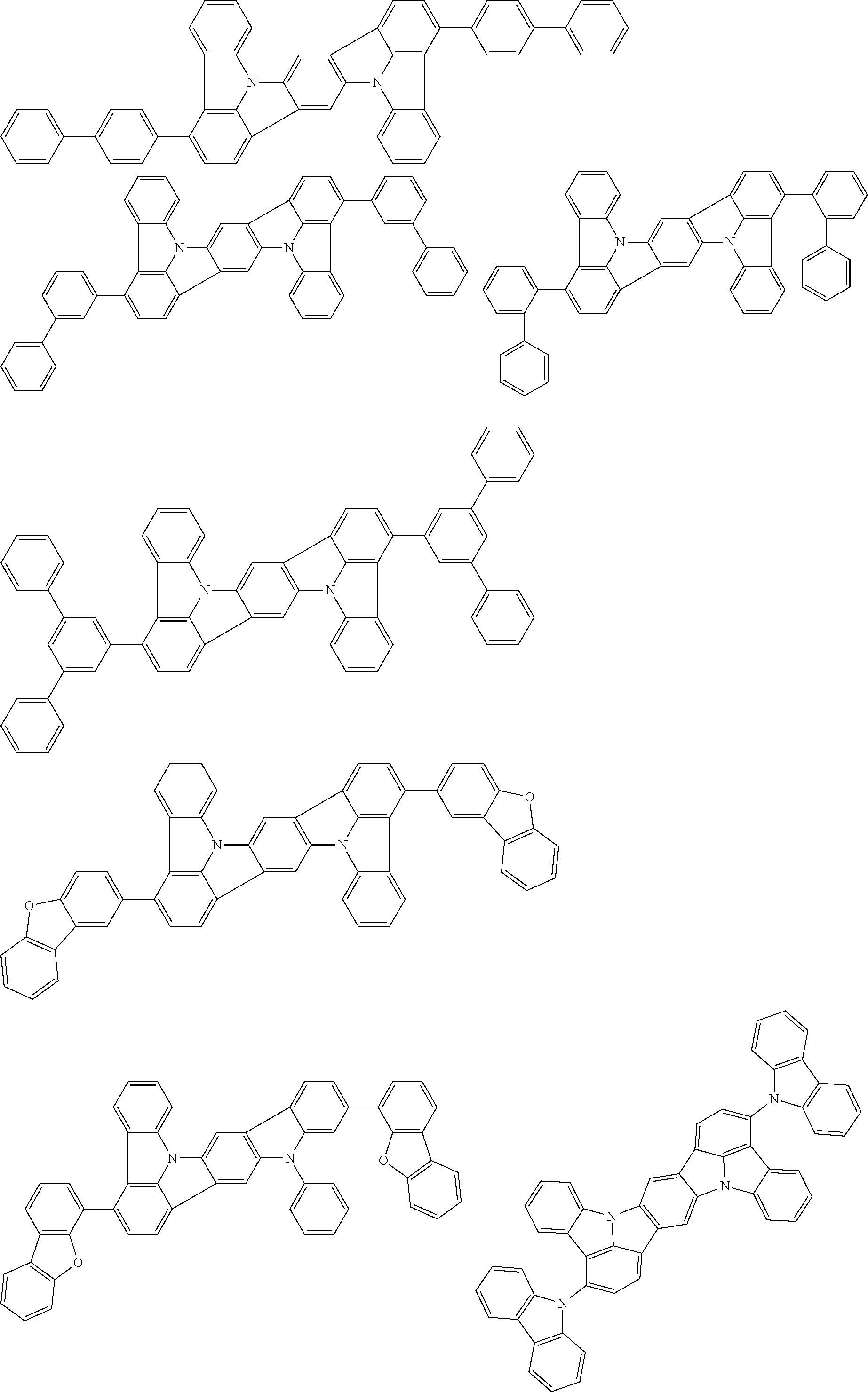

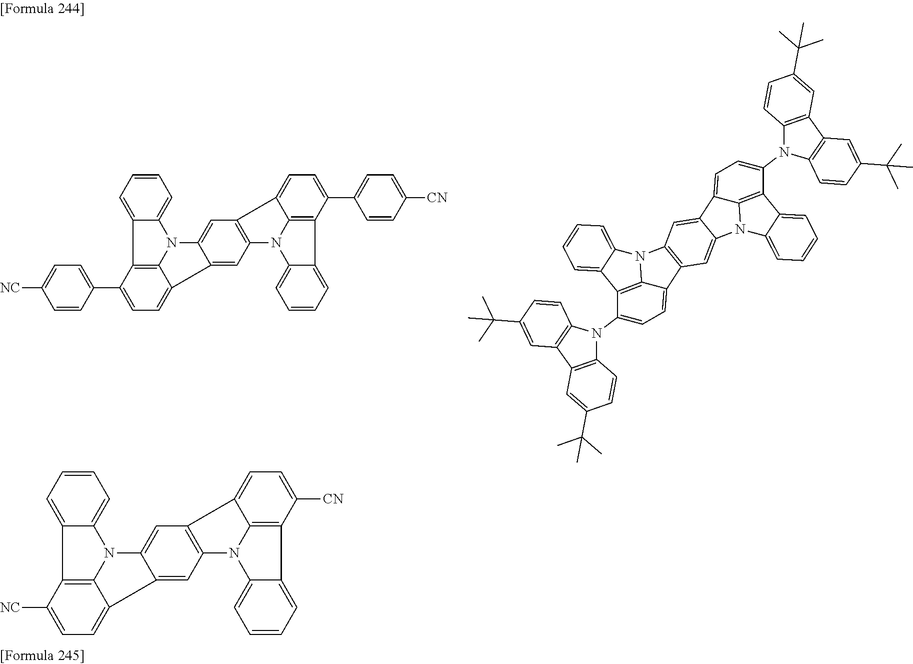 [SMILES: N#Cc1ccc(-c2ccc3c4cc5c(cc4n4c6ccccc6c2c34)c2ccc(-c3ccc(C#N)cc3)c3c4ccccc4n5c23)cc1 ; CC(C)(C)c1ccc2c(c1)c1cc(C(C)(C)C)ccc1n2-c1ccc2c3cc4c(cc3n3c5ccccc5c1c23)c1ccc(-n2c3ccc(C(C)(C)C)cc3c3cc(C(C)(C)C)ccc32)c2c3ccccc3n4c12 ; N#Cc1ccc2c3cc4c(cc3n3c5ccccc5c1c23)c1ccc(C#N)c2c3ccccc3n4c12]

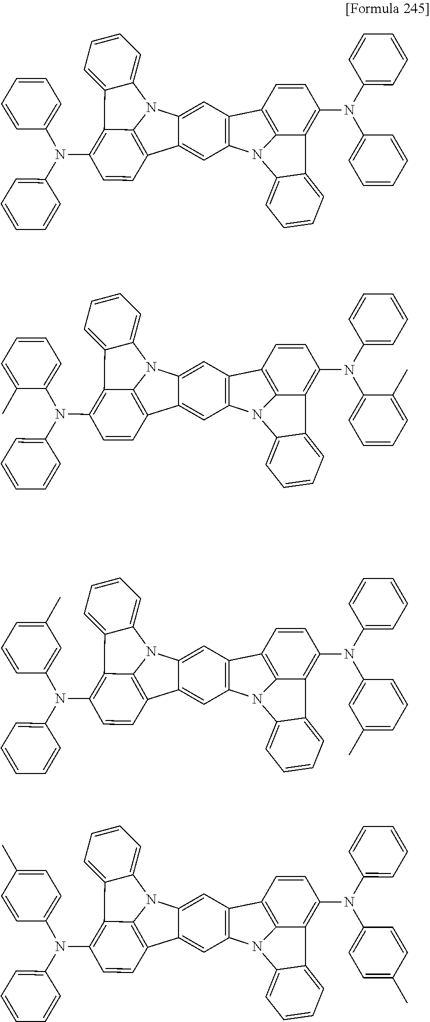 [SMILES: c1ccc(N(c2ccccc2)c2ccc3c4cc5c(cc4n4c6ccccc6c2c34)c2ccc(N(c3ccccc3)c3ccccc3)c3c4ccccc4n5c23)cc1 ; Cc1ccccc1N(c1ccccc1)c1ccc2c3cc4c(cc3n3c5ccccc5c1c23)c1ccc(N(c2ccccc2)c2ccccc2C)c2c3ccccc3n4c12 ; Cc1cccc(N(c2ccccc2)c2ccc3c4cc5c(cc4n4c6ccccc6c2c34)c2ccc(N(c3ccccc3)c3cccc(C)c3)c3c4ccccc4n5c23)c1 ; Cc1ccc(N(c2ccccc2)c2ccc3c4cc5c(cc4n4c6ccccc6c2c34)c2ccc(N(c3ccccc3)c3ccc(C)cc3)c3c4ccccc4n5c23)cc1]

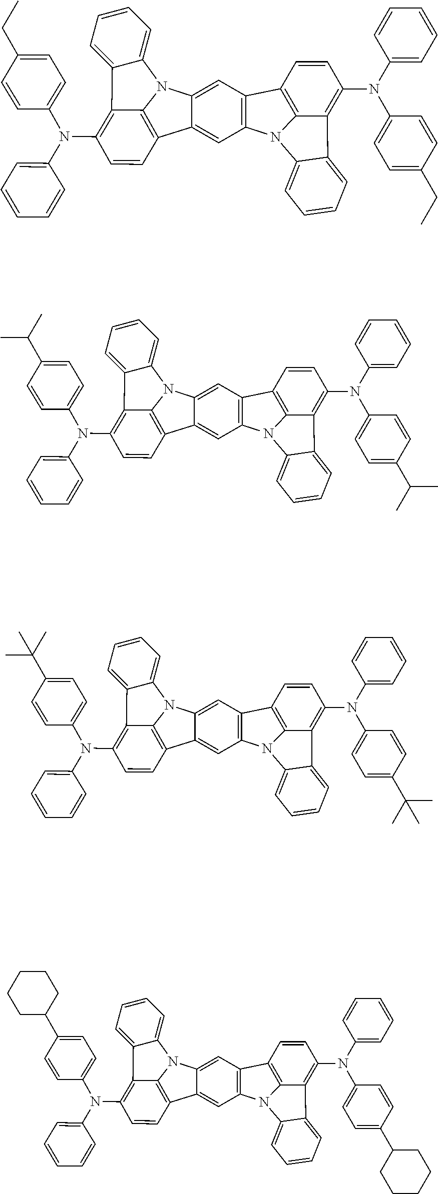

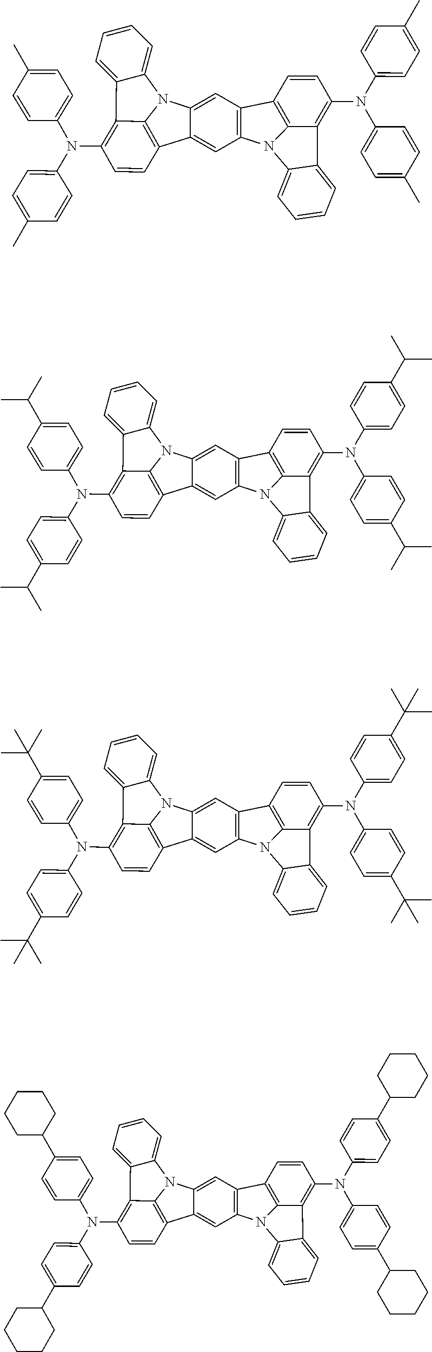

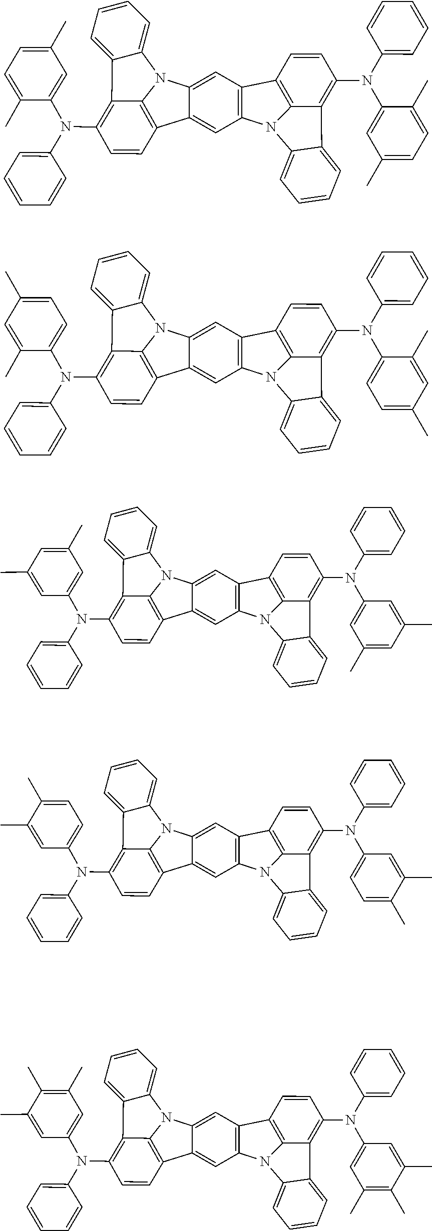

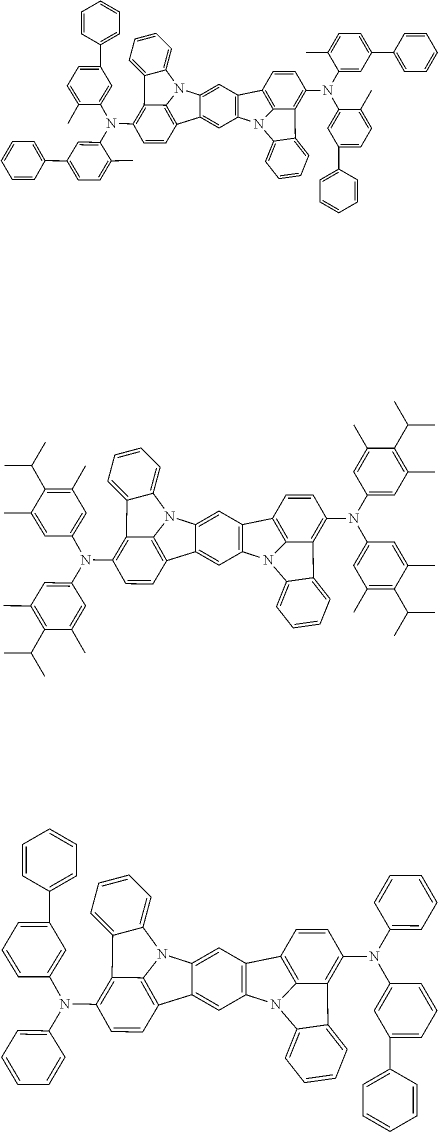

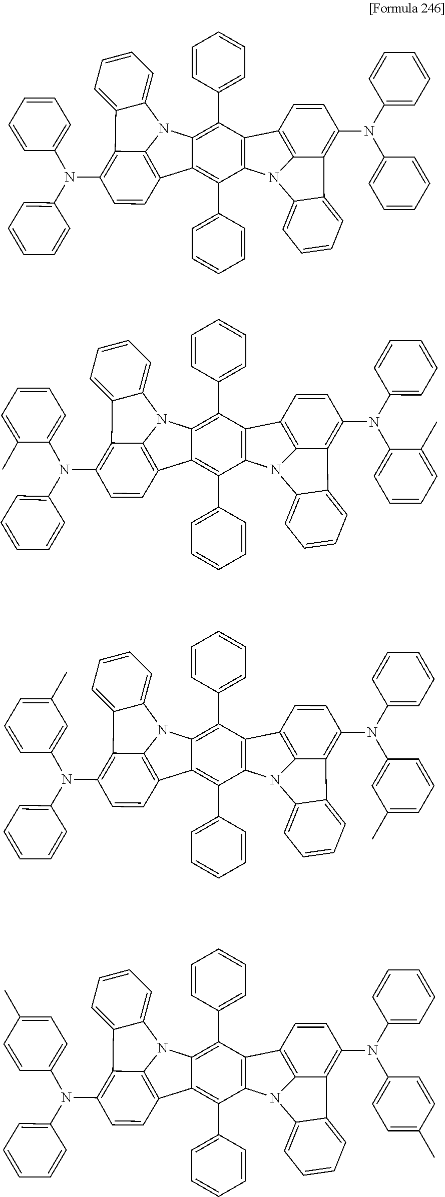 [SMILES: c1ccc(-c2c3c4ccc(N(c5ccccc5)c5ccccc5)c5c6ccccc6n(c3c(-c3ccccc3)c3c6ccc(N(c7ccccc7)c7ccccc7)c7c8ccccc8n(c23)c67)c45)cc1 ; Cc1ccccc1N(c1ccccc1)c1ccc2c3c(-c4ccccc4)c4c(c(-c5ccccc5)c3n3c5ccccc5c1c23)c1ccc(N(c2ccccc2)c2ccccc2C)c2c3ccccc3n4c12 ; Cc1cccc(N(c2ccccc2)c2ccc3c4c(-c5ccccc5)c5c(c(-c6ccccc6)c4n4c6ccccc6c2c34)c2ccc(N(c3ccccc3)c3cccc(C)c3)c3c4ccccc4n5c23)c1 ; Cc1ccc(N(c2ccccc2)c2ccc3c4c(-c5ccccc5)c5c(c(-c6ccccc6)c4n4c6ccccc6c2c34)c2ccc(N(c3ccccc3)c3ccc(C)cc3)c3c4ccccc4n5c23)cc1]

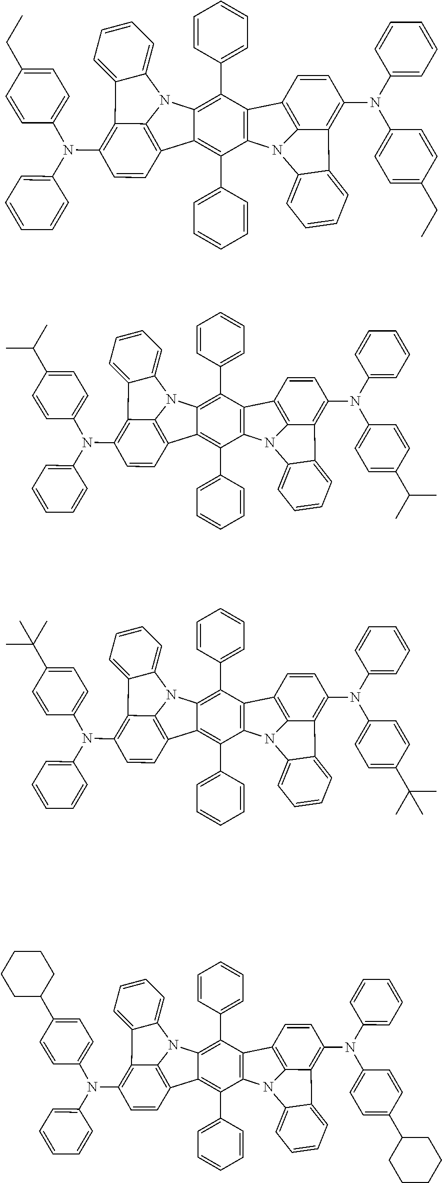

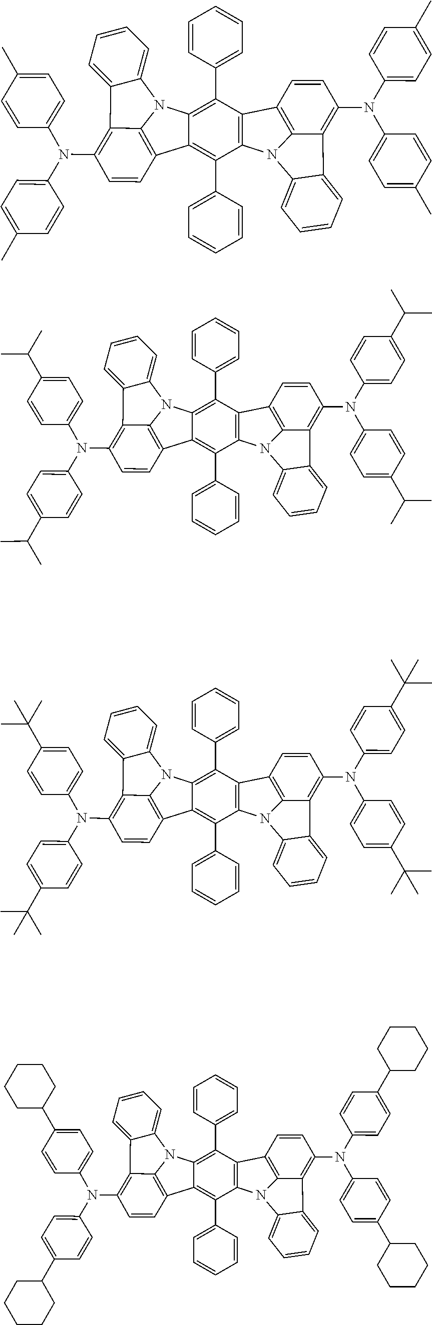

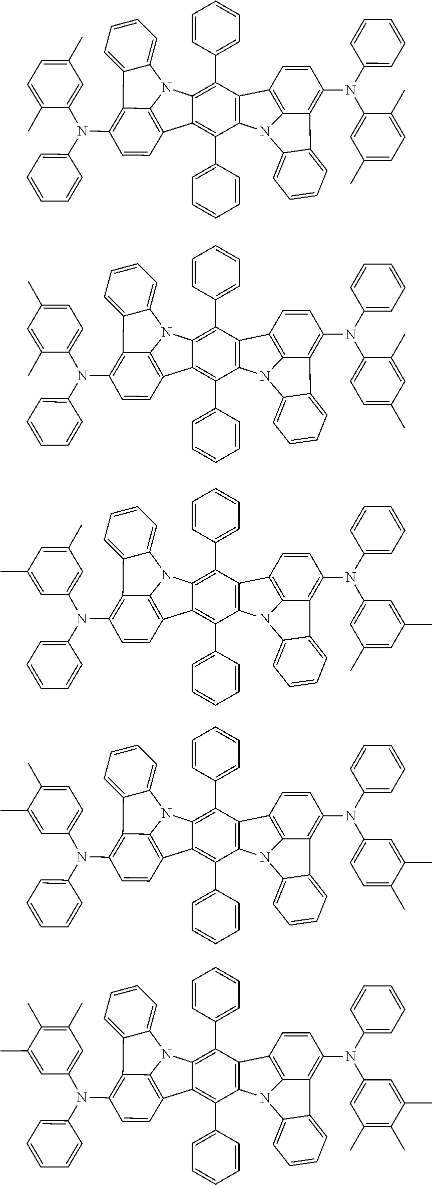

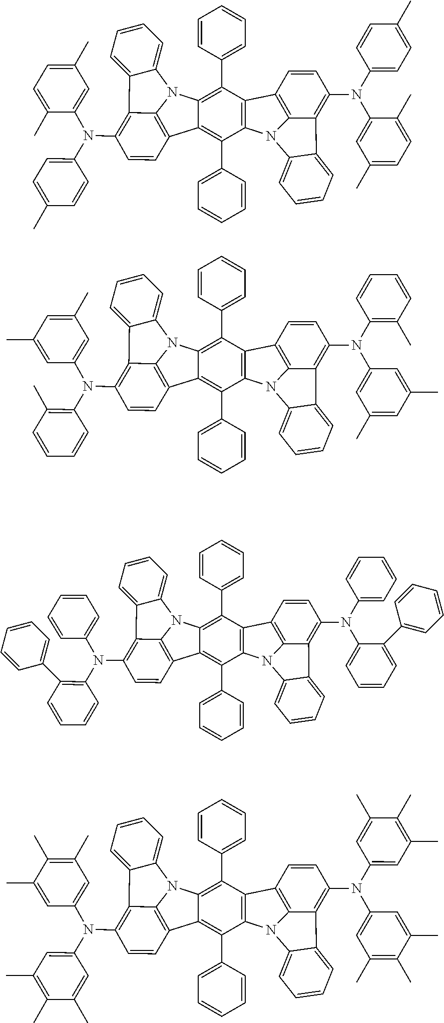

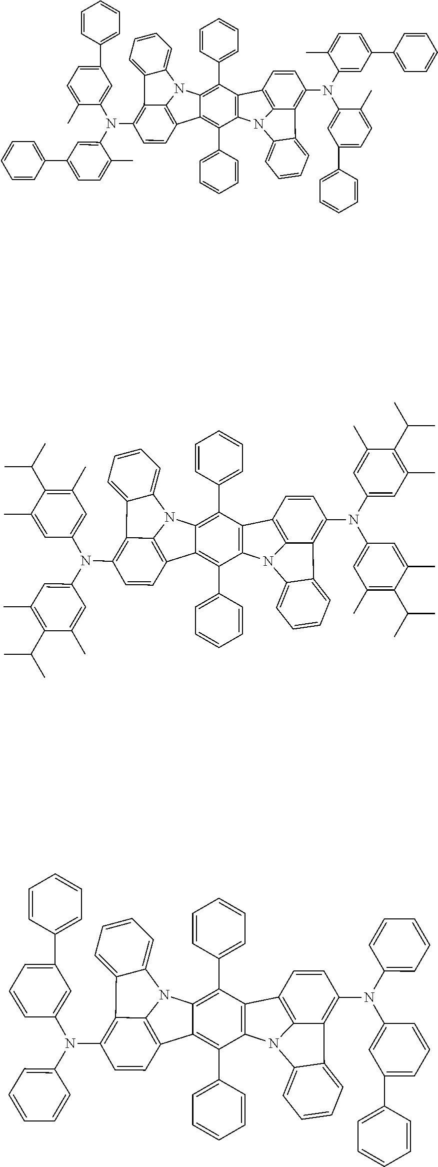

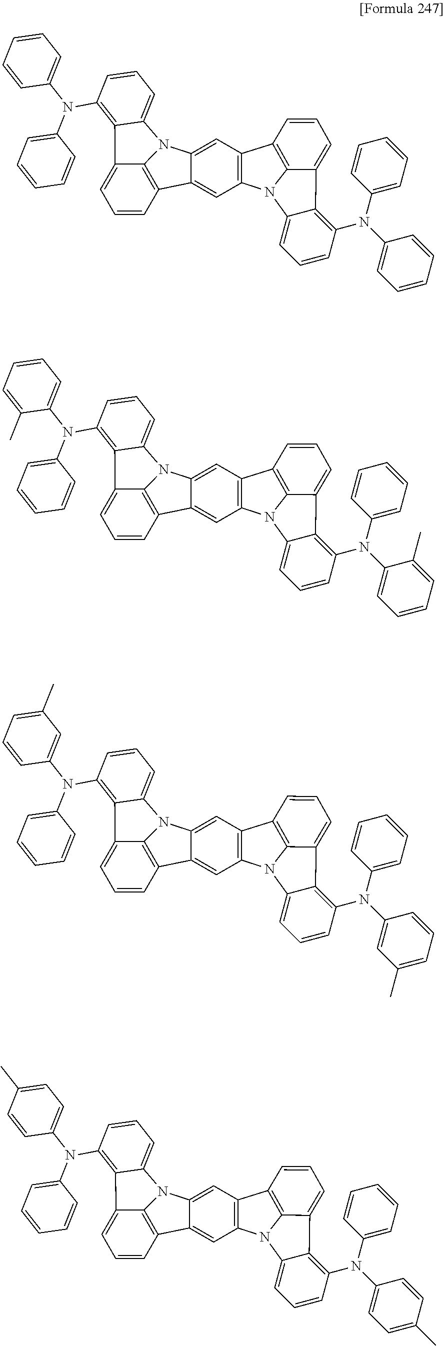

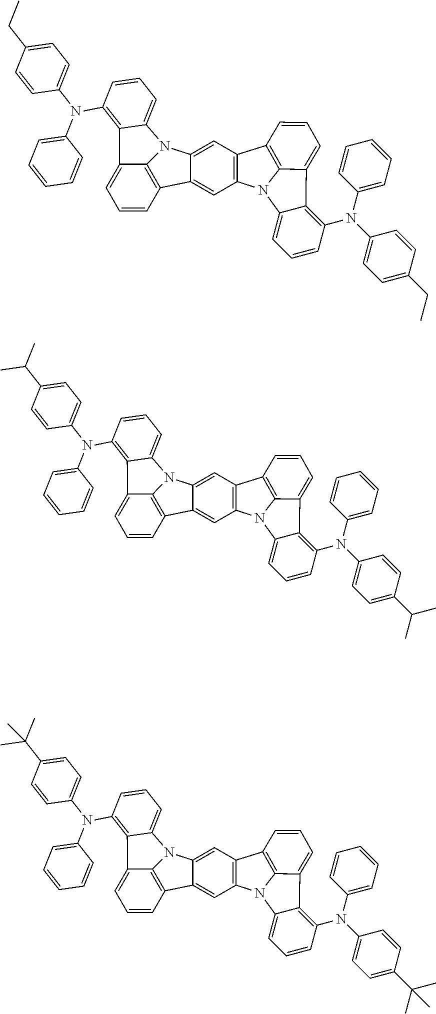

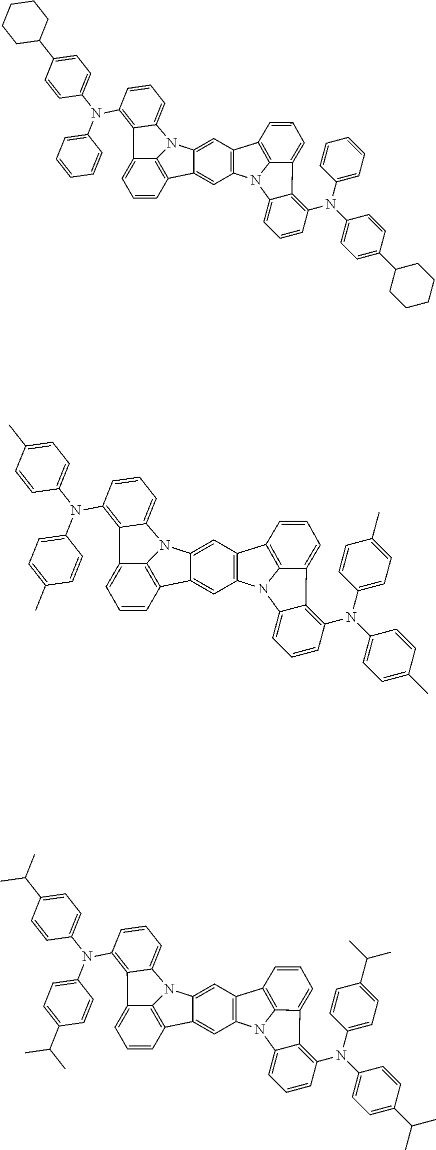

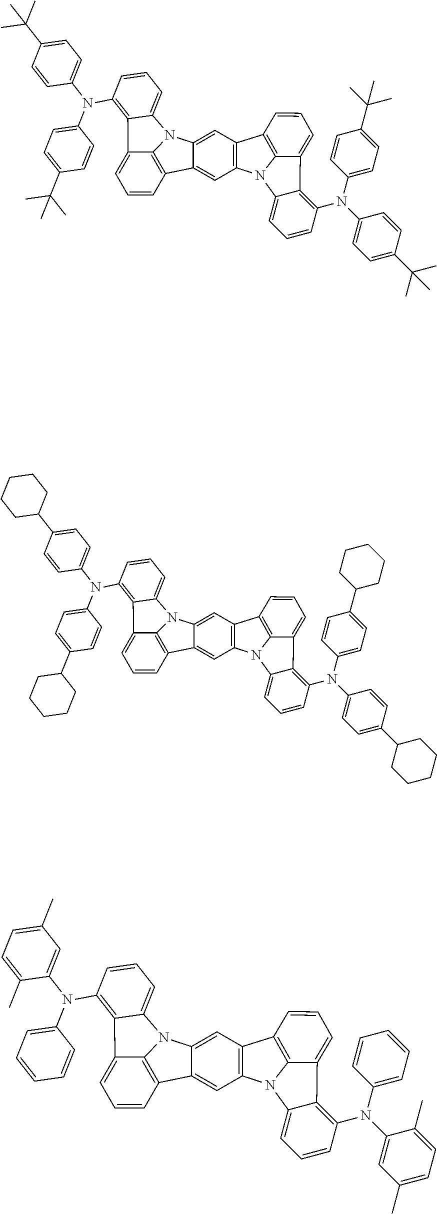 [SMILES: CC(C)(C)c1ccc(N(c2ccc(C(C)(C)C)cc2)c2cccc3c2c2cccc4c5cc6c(cc5n3c42)c2cccc3c4c(N(c5ccc(C(C)(C)C)cc5)c5ccc(C(C)(C)C)cc5)cccc4n6c23)cc1 ; c1cc2c3cc4c(cc3n3c5cccc(N(c6ccc(C7CCCCC7)cc6)c6ccc(C7CCCCC7)cc6)c5c(c1)c23)c1cccc2c3c(N(c5ccc(C6CCCCC6)cc5)c5ccc(C6CCCCC6)cc5)cccc3n4c12 ; Cc1ccc(C)c(N(c2ccccc2)c2cccc3c2c2cccc4c5cc6c(cc5n3c42)c2cccc3c4c(N(c5ccccc5)c5cc(C)ccc5C)cccc4n6c23)c1]

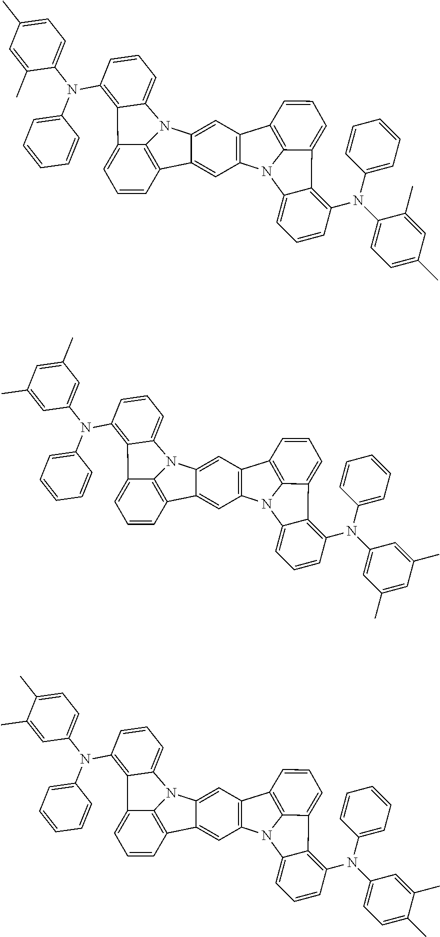

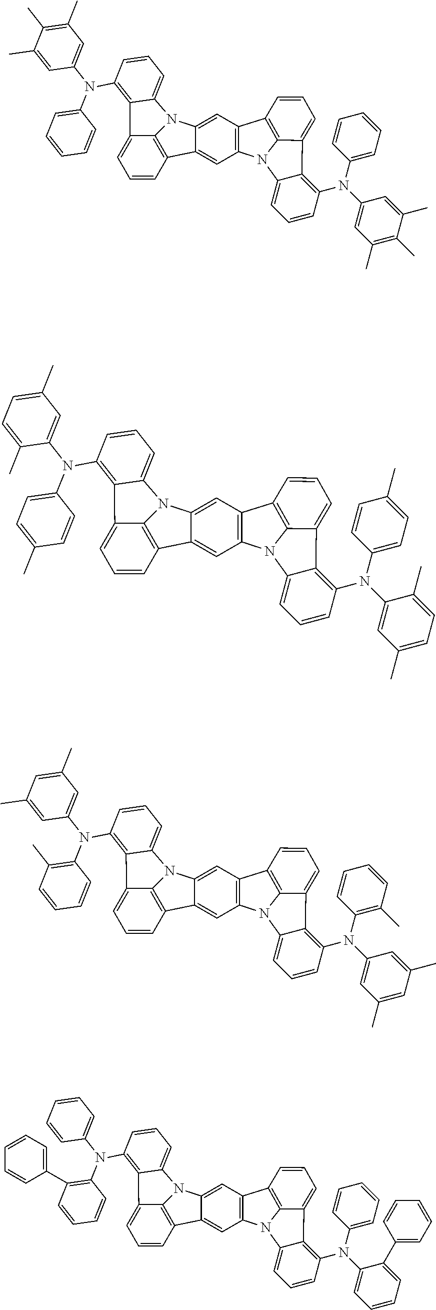

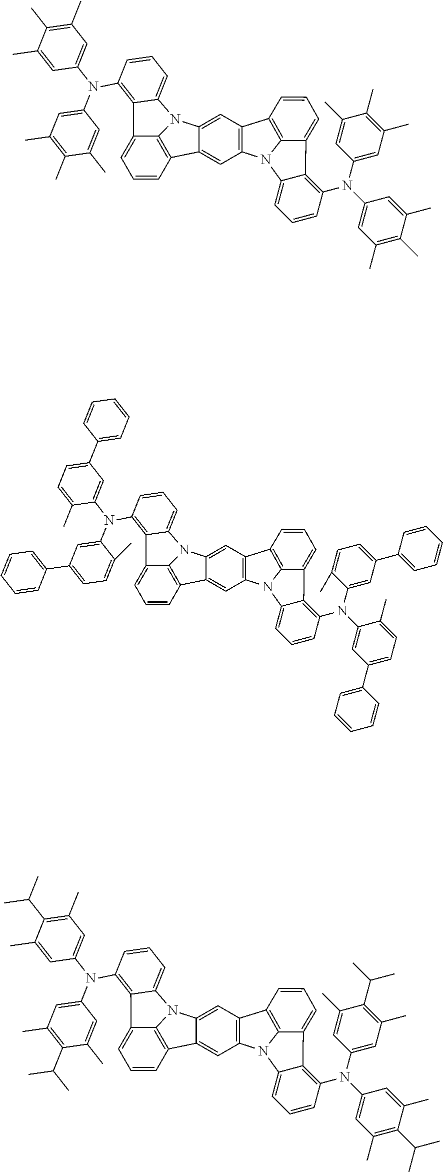

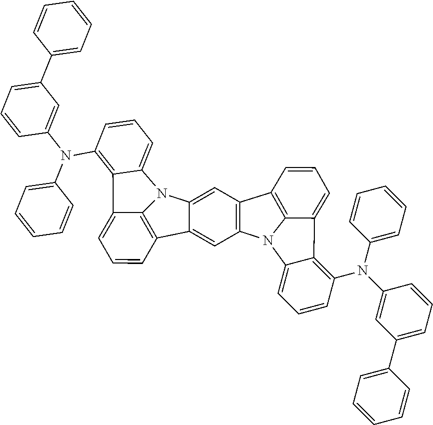 [SMILES: c1ccc(-c2cccc(N(c3ccccc3)c3cccc4c3c3cccc5c6cc7c(cc6n4c53)c3cccc4c5c(N(c6ccccc6)c6cccc(-c8ccccc8)c6)cccc5n7c34)c2)cc1]

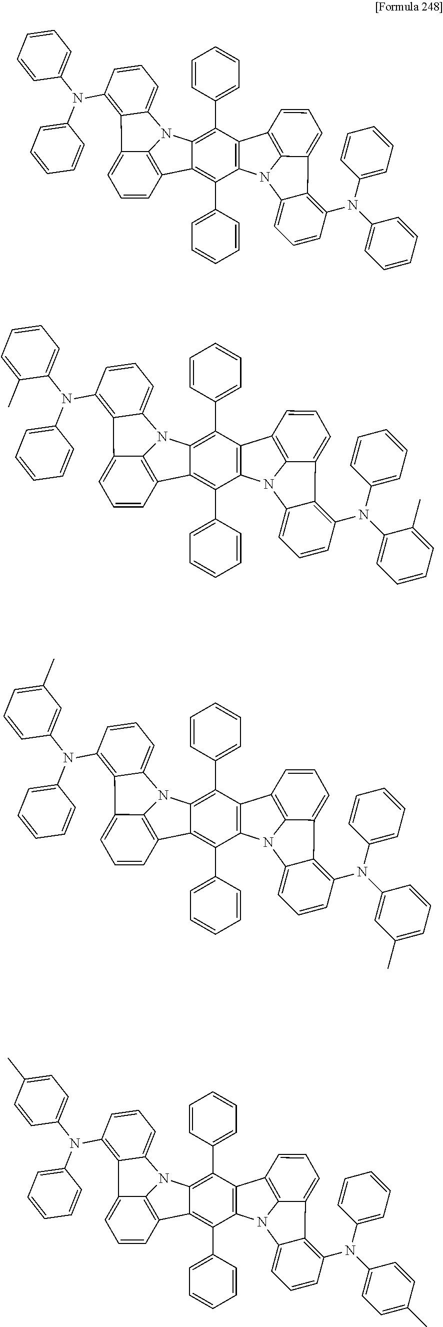

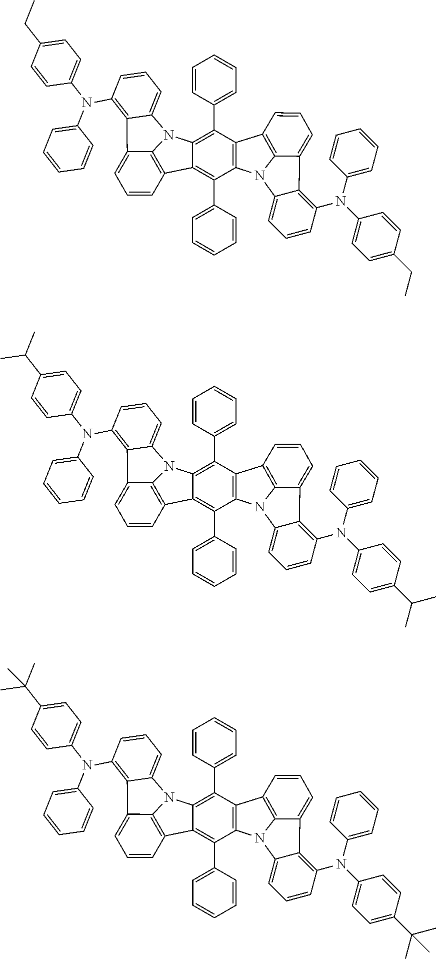

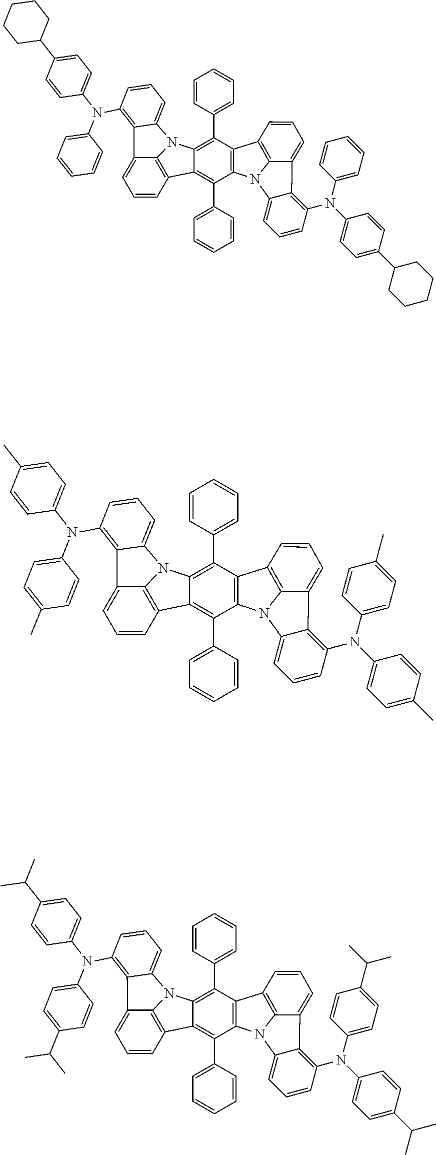

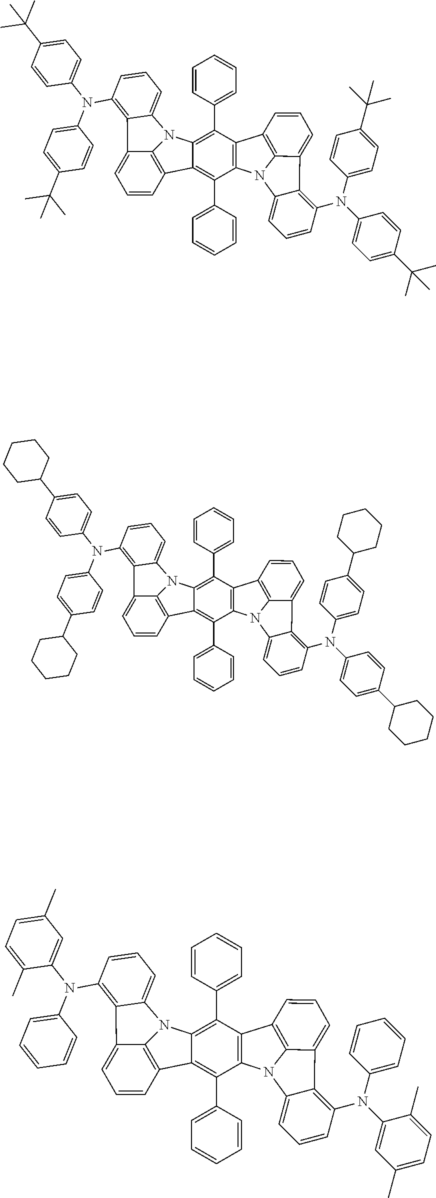 [SMILES: CC(C)(C)c1ccc(N(c2ccc(C(C)(C)C)cc2)c2cccc3c2c2cccc4c5c(-c6ccccc6)c6c(c(-c7ccccc7)c5n3c24)c2cccc3c4c(N(c5ccc(C(C)(C)C)cc5)c5ccc(C(C)(C)C)cc5)cccc4n6c32)cc1 ; c1ccc(-c2c3c4cccc5c6c(N(c7ccc(C8CCCCC8)cc7)c7ccc(C8CCCCC8)cc7)cccc6n(c3c(-c3ccccc3)c3c6cccc7c8c(N(c9ccc(C%10CCCCC%10)cc9)c9ccc(C%10CCCCC%10)cc9)cccc8n(c23)c76)c54)cc1 ; Cc1ccc(C)c(N(c2ccccc2)c2cccc3c2c2cccc4c5c(-c6ccccc6)c6c(c(-c7ccccc7)c5n3c24)c2cccc3c4c(N(c5ccccc5)c5cc(C)ccc5C)cccc4n6c32)c1]

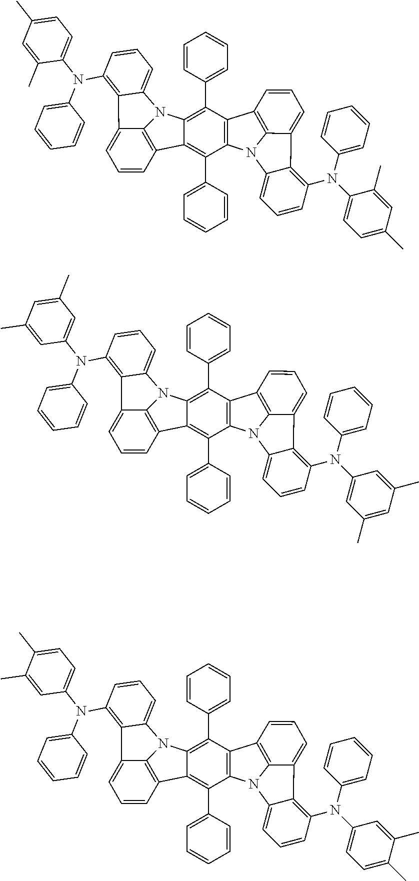

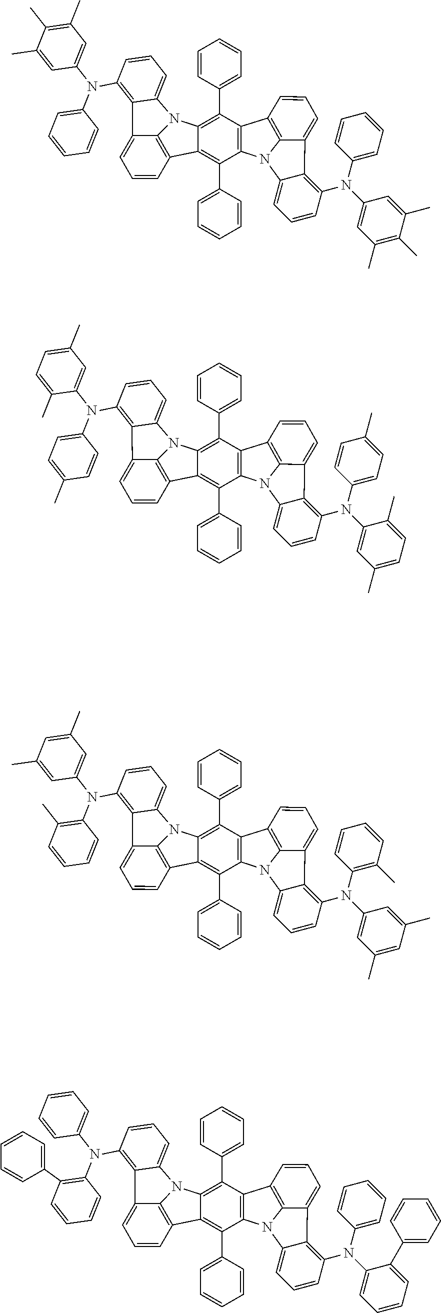

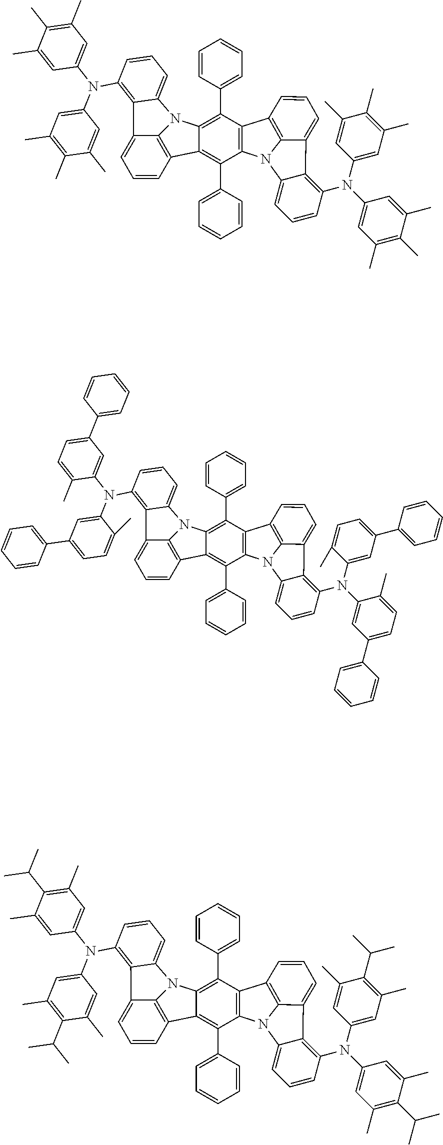

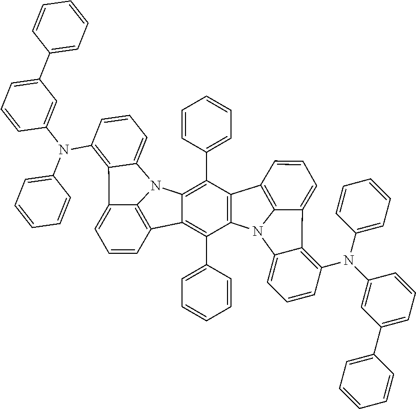 [SMILES: c1ccc(-c2cccc(N(c3ccccc3)c3cccc4c3c3cccc5c6c(-c7ccccc7)c7c(c(-c8ccccc8)c6n4c35)c3cccc4c5c(N(c6ccccc6)c6cccc(-c8ccccc8)c6)cccc5n7c43)c2)cc1]

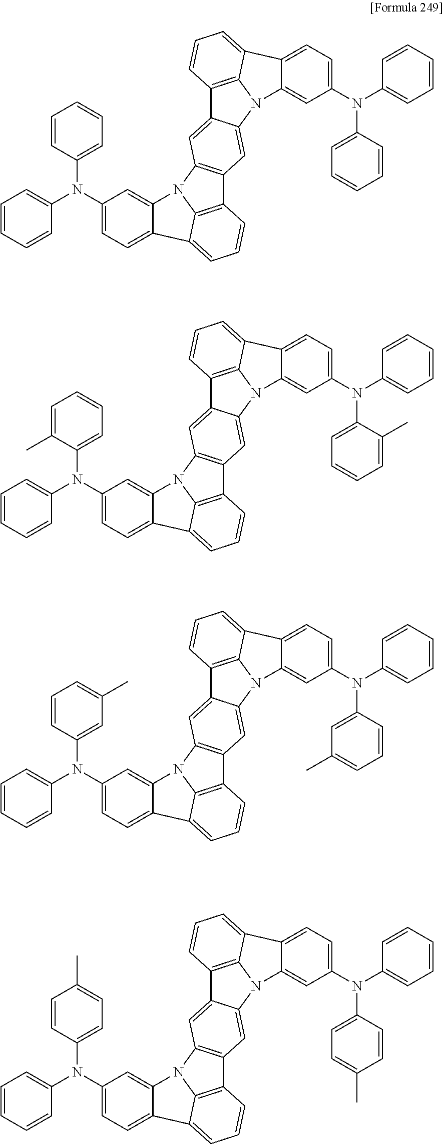 [SMILES: c1ccc(N(c2ccccc2)c2ccc3c4cccc5c6cc7c(cc6n(c3c2)c45)c2cccc3c4ccc(N(c5ccccc5)c5ccccc5)cc4n7c32)cc1 ; Cc1ccccc1N(c1ccccc1)c1ccc2c3cccc4c5cc6c(cc5n(c2c1)c34)c1cccc2c3ccc(N(c4ccccc4)c4ccccc4C)cc3n6c21 ; Cc1cccc(N(c2ccccc2)c2ccc3c4cccc5c6cc7c(cc6n(c3c2)c45)c2cccc3c4ccc(N(c5ccccc5)c5cccc(C)c5)cc4n7c32)c1 ; Cc1ccc(N(c2ccccc2)c2ccc3c4cccc5c6cc7c(cc6n(c3c2)c45)c2cccc3c4ccc(N(c5ccccc5)c5ccc(C)cc5)cc4n7c32)cc1]

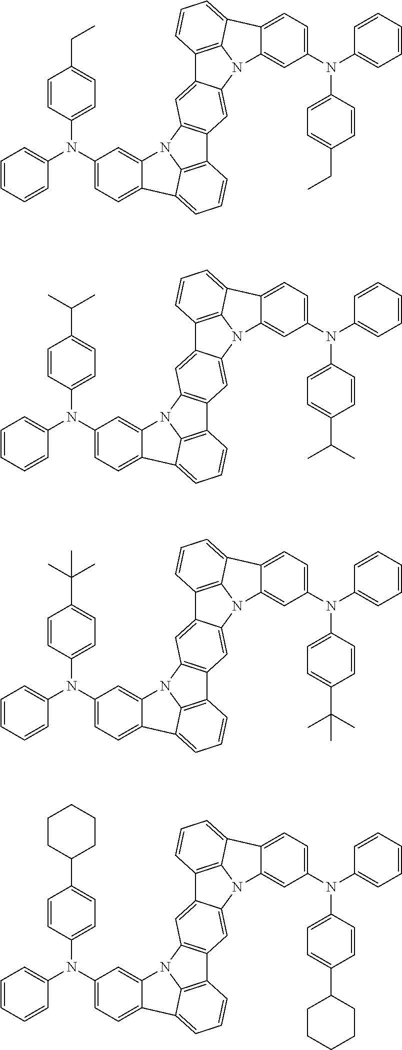 [SMILES: CCc1ccc(N(c2ccccc2)c2ccc3c4cccc5c6cc7c(cc6n(c3c2)c45)c2cccc3c4ccc(N(c5ccccc5)c5ccc(CC)cc5)cc4n7c32)cc1 ; CC(C)c1ccc(N(c2ccccc2)c2ccc3c4cccc5c6cc7c(cc6n(c3c2)c45)c2cccc3c4ccc(N(c5ccccc5)c5ccc(C(C)C)cc5)cc4n7c32)cc1 ; CC(C)(C)c1ccc(N(c2ccccc2)c2ccc3c4cccc5c6cc7c(cc6n(c3c2)c45)c2cccc3c4ccc(N(c5ccccc5)c5ccc(C(C)(C)C)cc5)cc4n7c32)cc1 ; c1ccc(N(c2ccc(C3CCCCC3)cc2)c2ccc3c4cccc5c6cc7c(cc6n(c3c2)c45)c2cccc3c4ccc(N(c5ccccc5)c5ccc(C6CCCCC6)cc5)cc4n7c32)cc1]

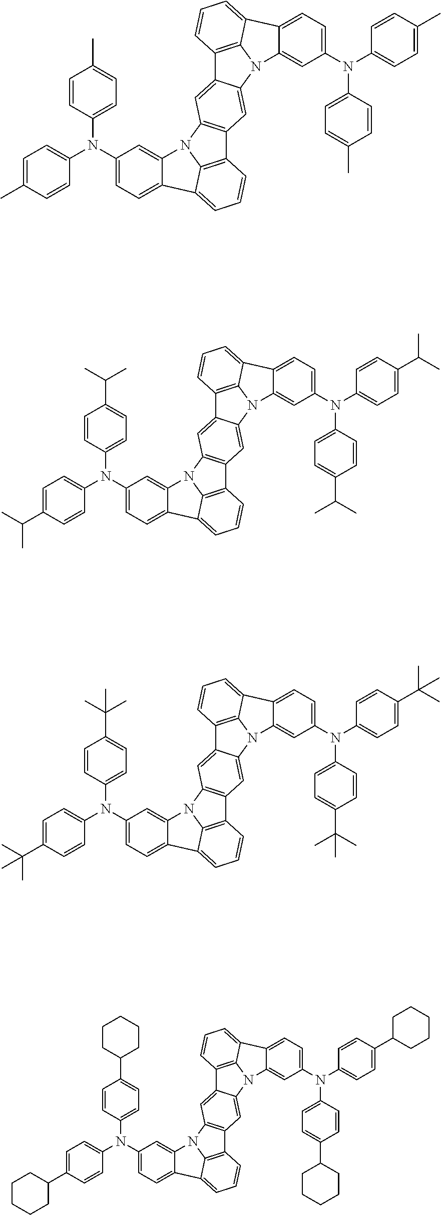

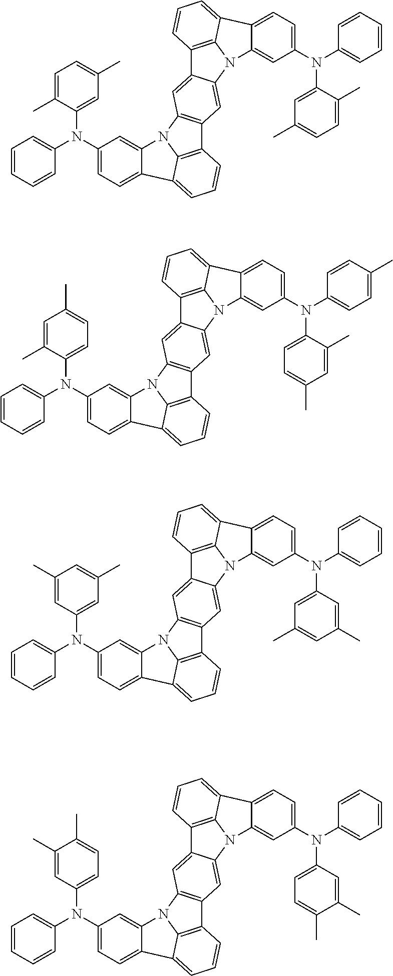 [SMILES: Cc1ccc(C)c(N(c2ccccc2)c2ccc3c4cccc5c6cc7c(cc6n(c3c2)c45)c2cccc3c4ccc(N(c5ccccc5)c5cc(C)ccc5C)cc4n7c32)c1 ; Cc1ccc(N(c2ccc3c4cccc5c6cc7c(cc6n(c3c2)c45)c2cccc3c4ccc(N(c5ccccc5)c5ccc(C)cc5C)cc4n7c32)c2ccc(C)cc2C)cc1 ; Cc1cc(C)cc(N(c2ccccc2)c2ccc3c4cccc5c6cc7c(cc6n(c3c2)c45)c2cccc3c4ccc(N(c5ccccc5)c5cc(C)cc(C)c5)cc4n7c32)c1 ; Cc1ccc(N(c2ccccc2)c2ccc3c4cccc5c6cc7c(cc6n(c3c2)c45)c2cccc3c4ccc(N(c5ccccc5)c5ccc(C)c(C)c5)cc4n7c32)cc1C]

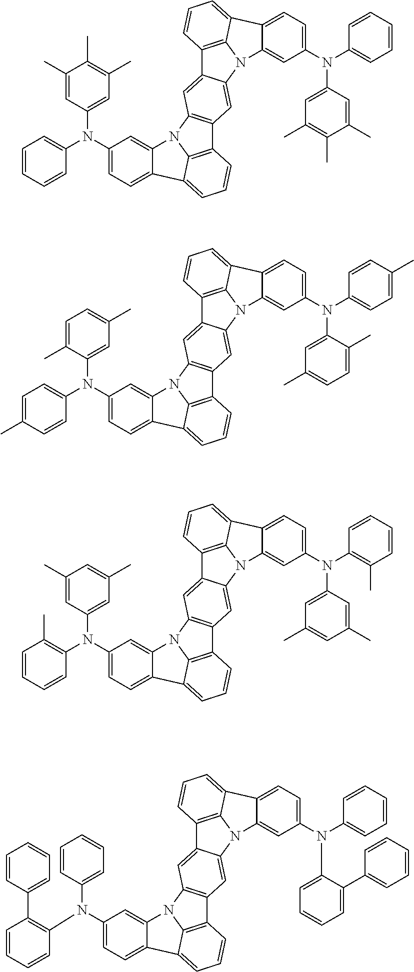

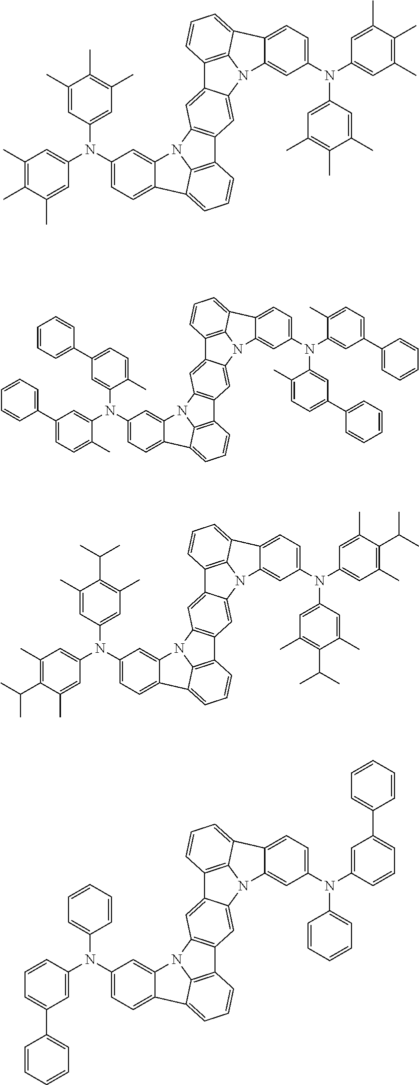

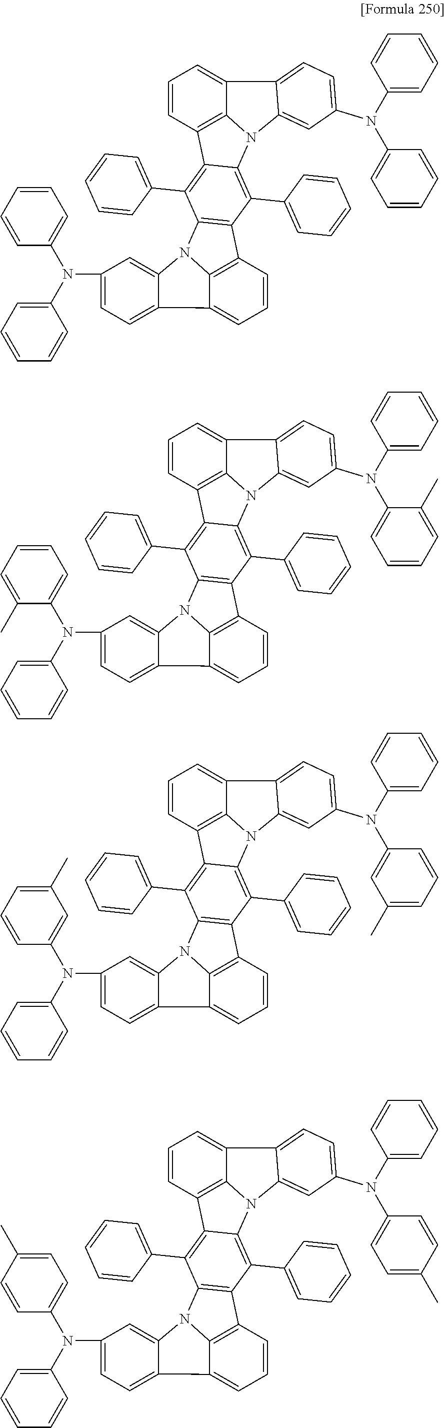 [SMILES: c1ccc(-c2c3c4cccc5c6ccc(N(c7ccccc7)c7ccccc7)cc6n(c3c(-c3ccccc3)c3c6cccc7c8ccc(N(c9ccccc9)c9ccccc9)cc8n(c23)c76)c54)cc1 ; Cc1ccccc1N(c1ccccc1)c1ccc2c3cccc4c5c(-c6ccccc6)c6c(c(-c7ccccc7)c5n(c2c1)c34)c1cccc2c3ccc(N(c4ccccc4)c4ccccc4C)cc3n6c21 ; Cc1cccc(N(c2ccccc2)c2ccc3c4cccc5c6c(-c7ccccc7)c7c(c(-c8ccccc8)c6n(c3c2)c45)c2cccc3c4ccc(N(c5ccccc5)c5cccc(C)c5)cc4n7c32)c1 ; Cc1ccc(N(c2ccccc2)c2ccc3c4cccc5c6c(-c7ccccc7)c7c(c(-c8ccccc8)c6n(c3c2)c45)c2cccc3c4ccc(N(c5ccccc5)c5ccc(C)cc5)cc4n7c32)cc1]

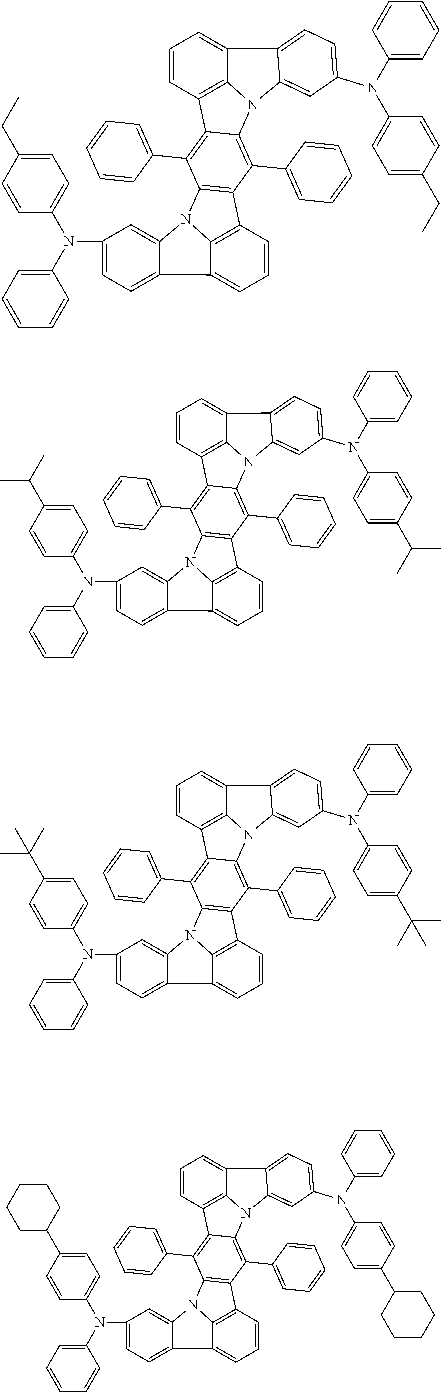

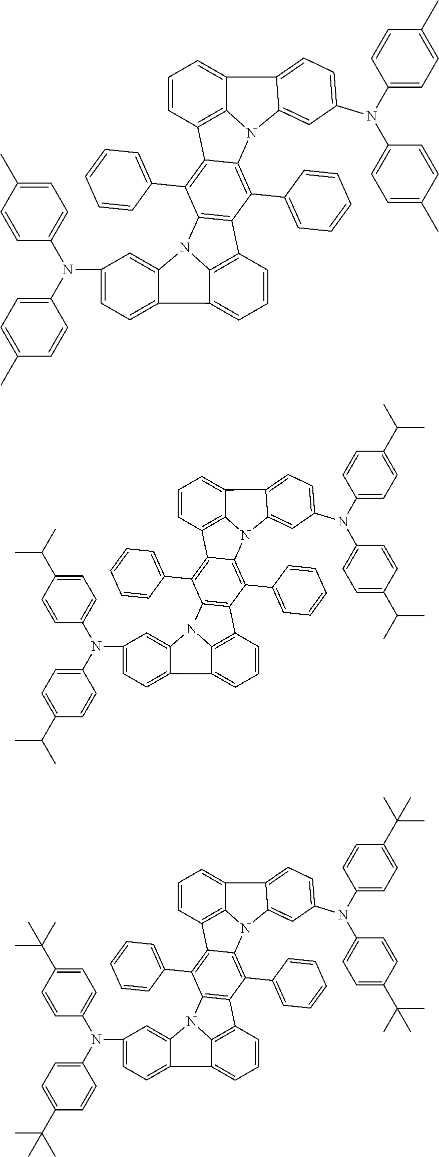

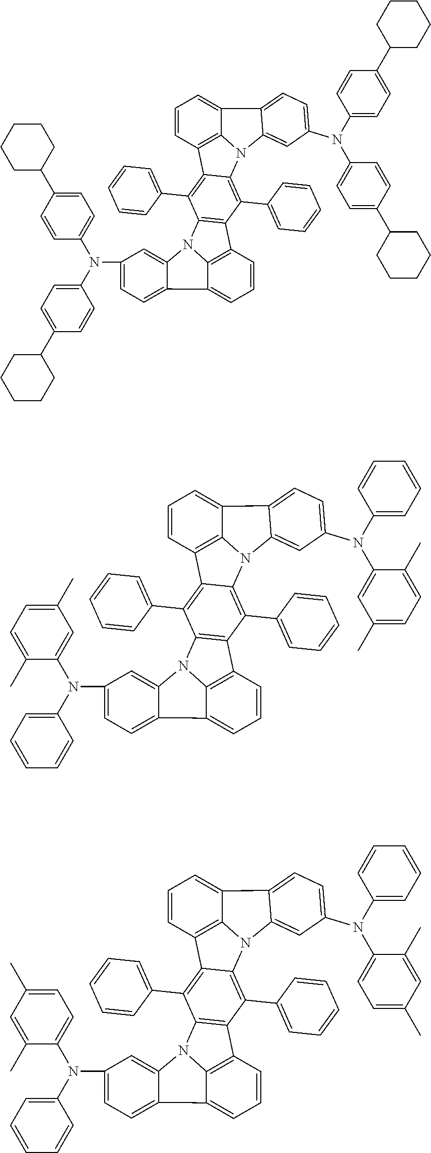 [SMILES: c1ccc(-c2c3c4cccc5c6ccc(N(c7ccc(C8CCCCC8)cc7)c7ccc(C8CCCCC8)cc7)cc6n(c3c(-c3ccccc3)c3c6cccc7c8ccc(N(c9ccc(C%10CCCCC%10)cc9)c9ccc(C%10CCCCC%10)cc9)cc8n(c23)c76)c54)cc1 ; Cc1ccc(C)c(N(c2ccccc2)c2ccc3c4cccc5c6c(-c7ccccc7)c7c(c(-c8ccccc8)c6n(c3c2)c45)c2cccc3c4ccc(N(c5ccccc5)c5cc(C)ccc5C)cc4n7c32)c1 ; Cc1ccc(N(c2ccccc2)c2ccc3c4cccc5c6c(-c7ccccc7)c7c(c(-c8ccccc8)c6n(c3c2)c45)c2cccc3c4ccc(N(c5ccccc5)c5ccc(C)cc5C)cc4n7c32)c(C)c1]

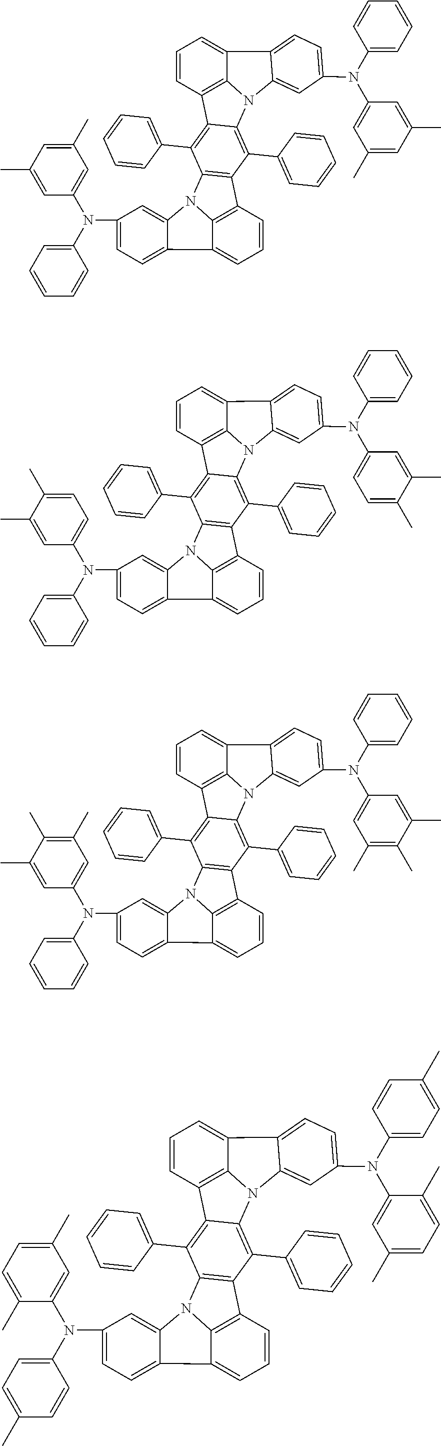

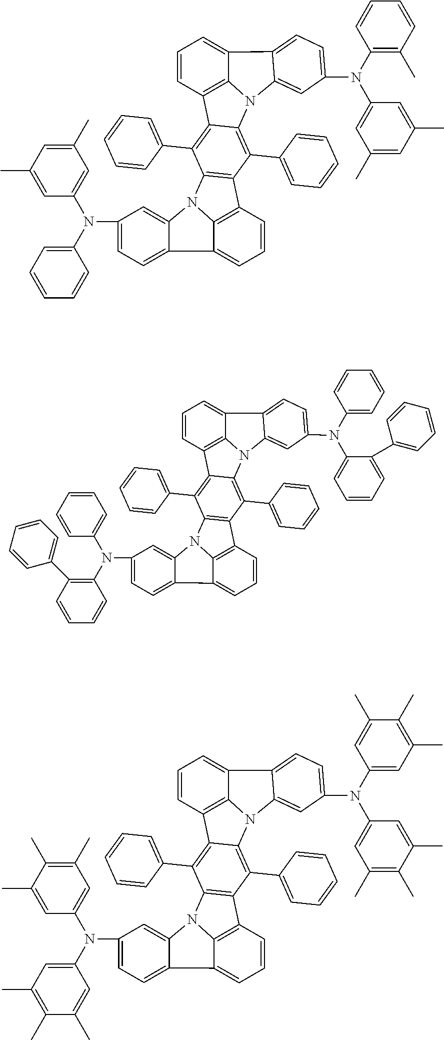

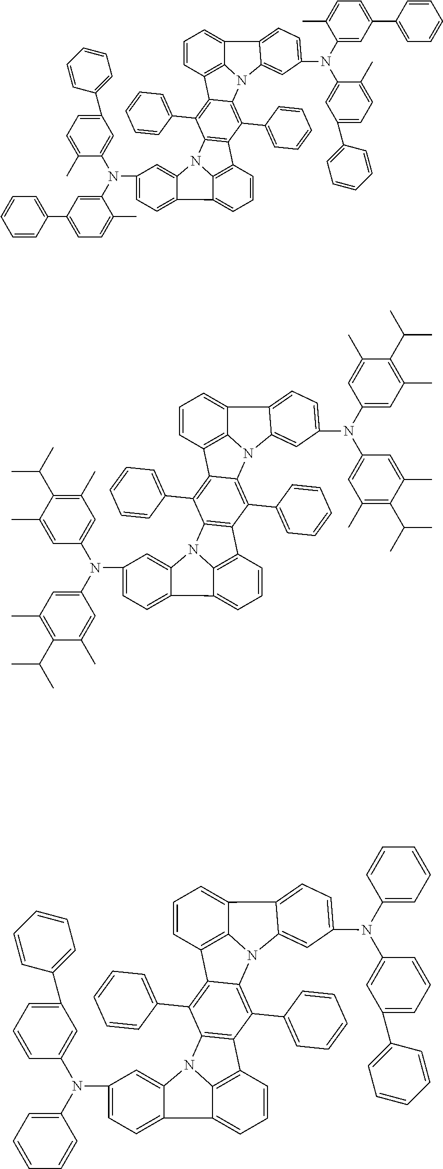 [SMILES: Cc1ccc(-c2ccccc2)cc1N(c1ccc2c3cccc4c5c(-c6ccccc6)c6c(c(-c7ccccc7)c5n(c2c1)c34)c1cccc2c3ccc(N(c4cc(-c5ccccc5)ccc4C)c4cc(-c5ccccc5)ccc4C)cc3n6c21)c1cc(-c2ccccc2)ccc1C ; Cc1cc(N(c2cc(C)c(C(C)C)c(C)c2)c2ccc3c4cccc5c6c(-c7ccccc7)c7c(c(-c8ccccc8)c6n(c3c2)c45)c2cccc3c4ccc(N(c5cc(C)c(C(C)C)c(C)c5)c5cc(C)c(C(C)C)c(C)c5)cc4n7c32)cc(C)c1C(C)C ; c1ccc(-c2cccc(N(c3ccccc3)c3ccc4c5cccc6c7c(-c8ccccc8)c8c(c(-c9ccccc9)c7n(c4c3)c56)c3cccc4c5ccc(N(c6ccccc6)c6cccc(-c7ccccc7)c6)cc5n8c43)c2)cc1]

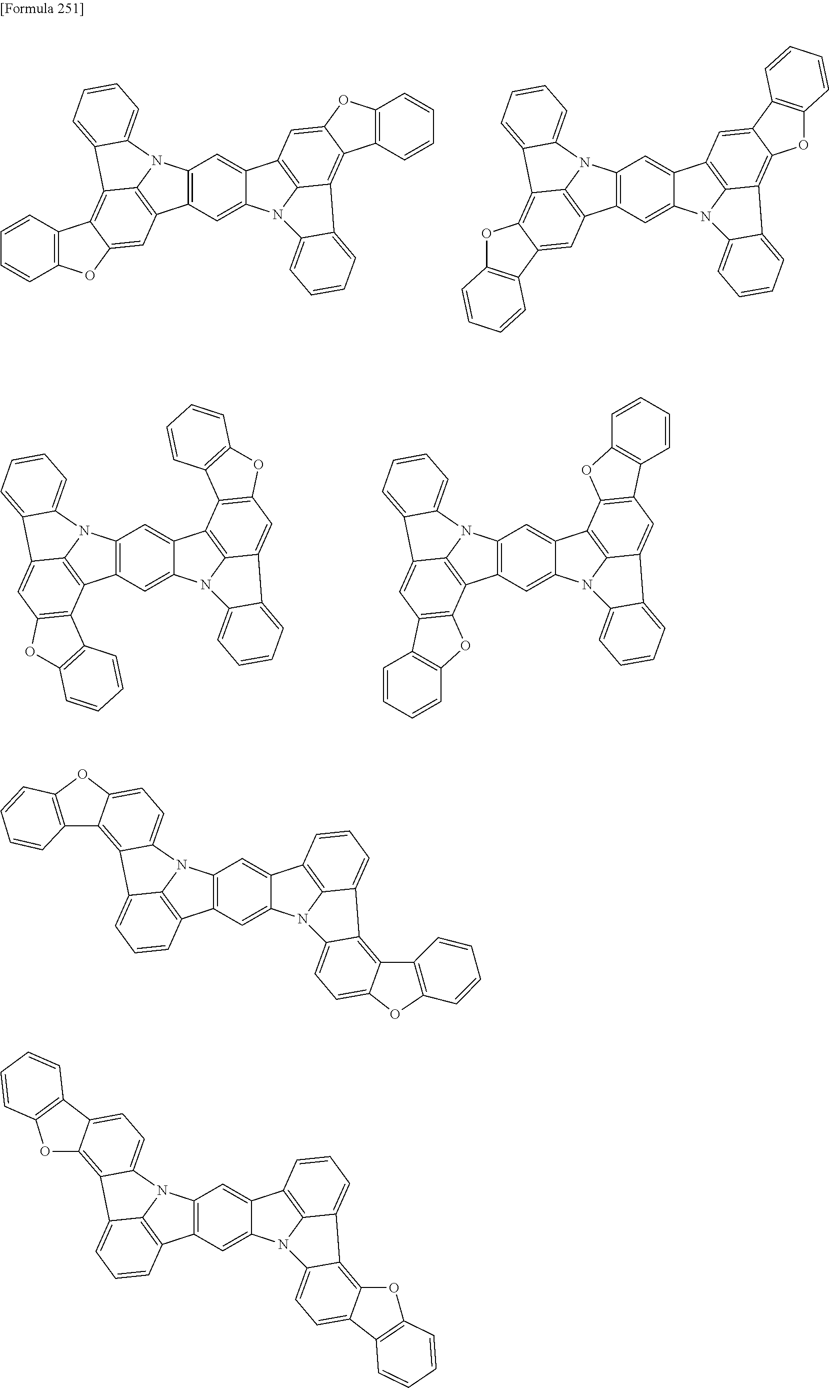 [SMILES: c1ccc2c(c1)oc1cc3c4cc5c(cc4n4c6ccccc6c(c12)c34)c1cc2oc3ccccc3c2c2c3ccccc3n5c12 ; c1ccc2c(c1)oc1c2cc2c3cc4c(cc3n3c5ccccc5c1c23)c1cc2c3ccccc3oc2c2c3ccccc3n4c12 ; c1ccc2c(c1)oc1cc3c4ccccc4n4c5cc6c7c8c(cc9c%10ccccc%10n(c6cc5c(c12)c34)c97)oc1ccccc18 ; c1ccc2c(c1)oc1c2cc2c3ccccc3n3c4cc5c6c7oc8ccccc8c7cc7c8ccccc8n(c5cc4c1c23)c76 ; c1ccc2c(c1)oc1ccc3c(c4cccc5c6cc7c(cc6n3c54)c3cccc4c5c6c(ccc5n7c34)oc3ccccc36)c12 ; c1ccc2c(c1)oc1c2ccc2c1c1cccc3c4cc5c(cc4n2c31)c1cccc2c3c4oc6ccccc6c4ccc3n5c12]

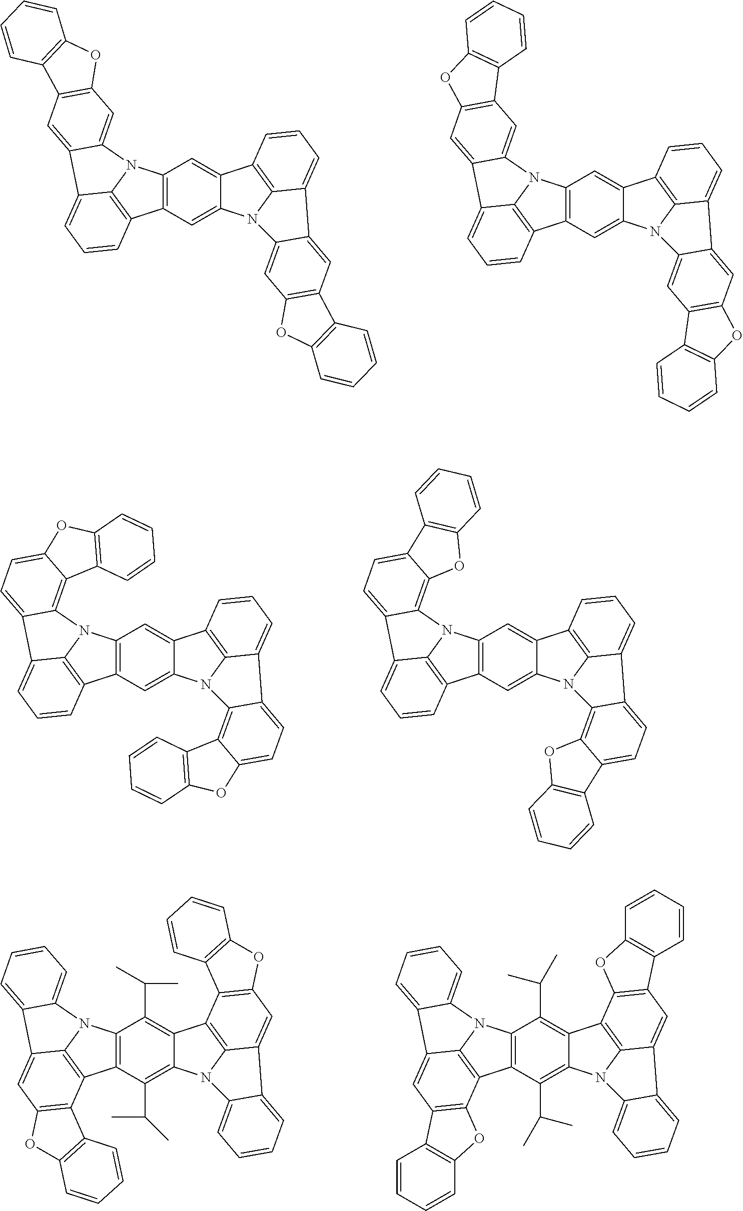

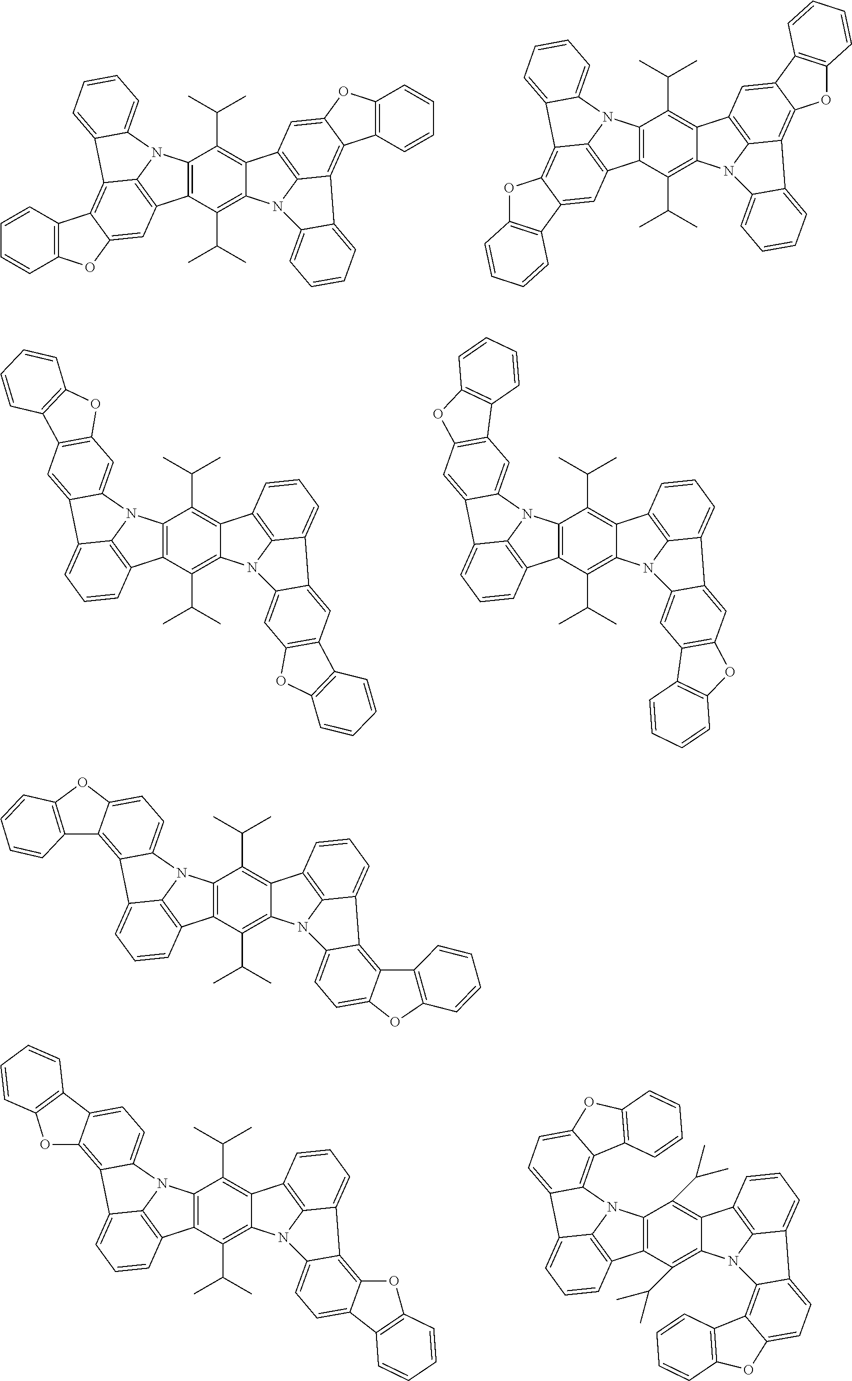

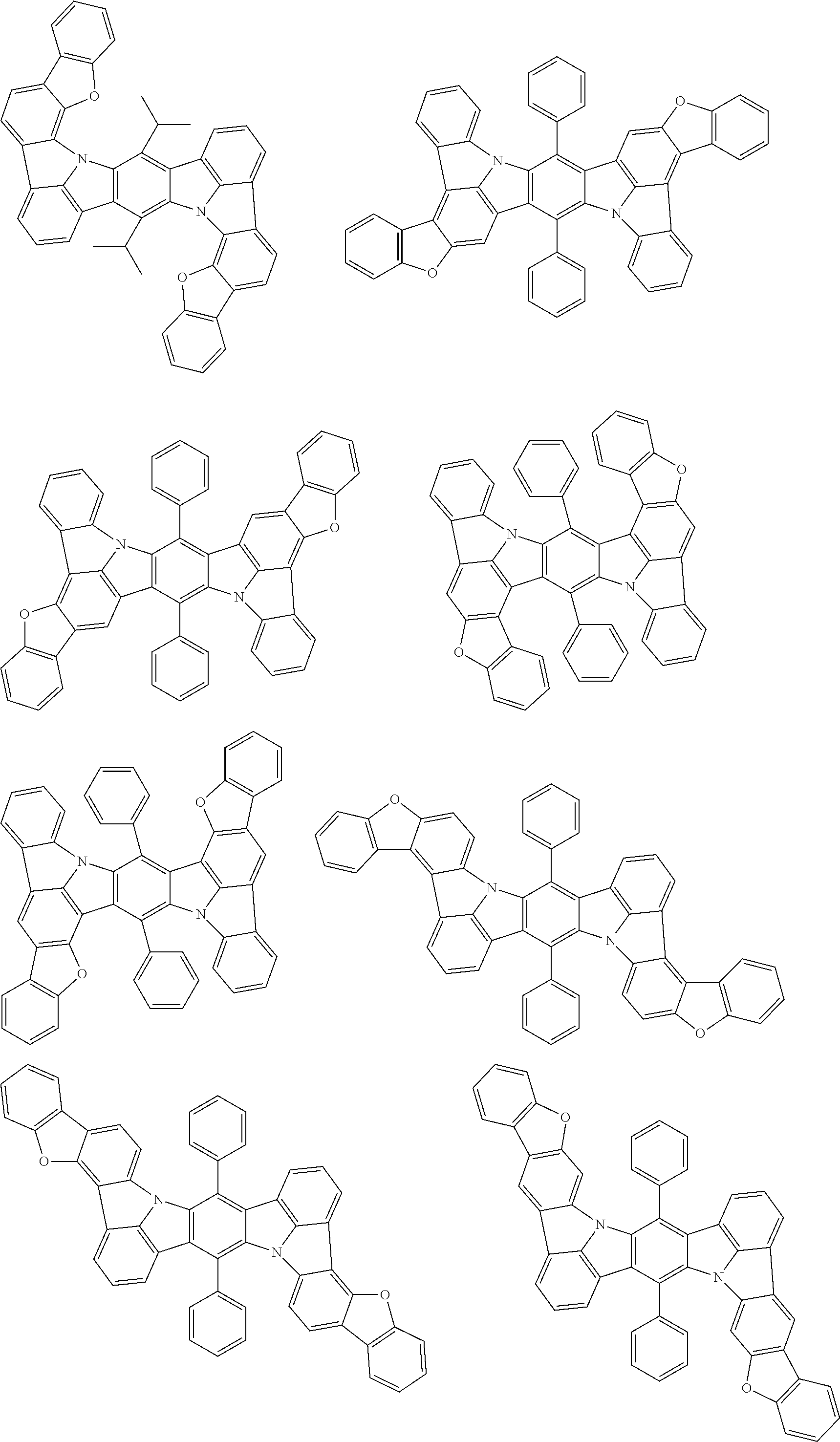

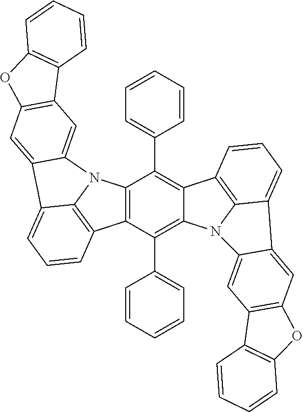 [SMILES: c1ccc(-c2c3c4cccc5c6cc7oc8ccccc8c7cc6n(c3c(-c3ccccc3)c3c6cccc7c8cc9oc%10ccccc%10c9cc8n(c23)c76)c54)cc1]

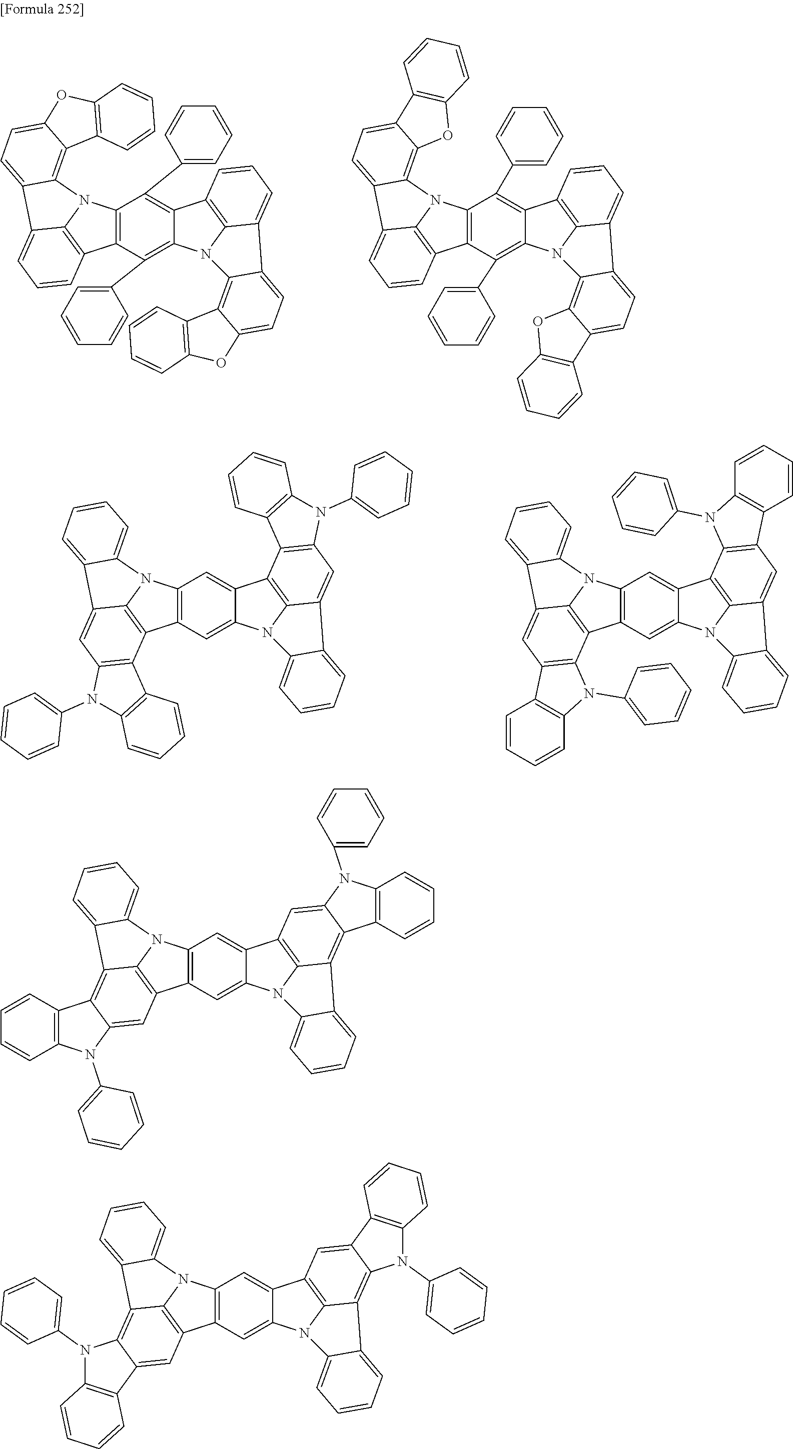

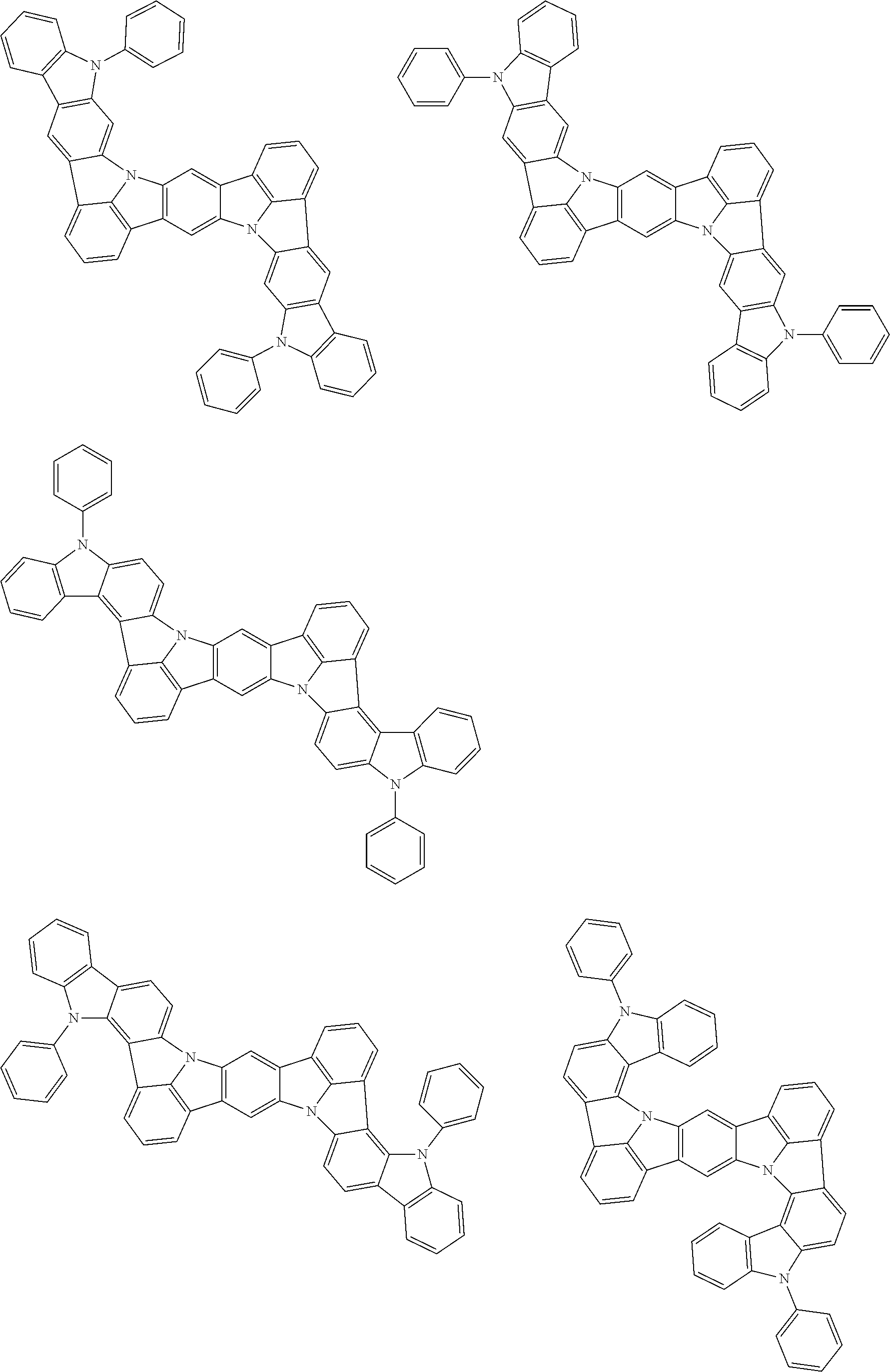

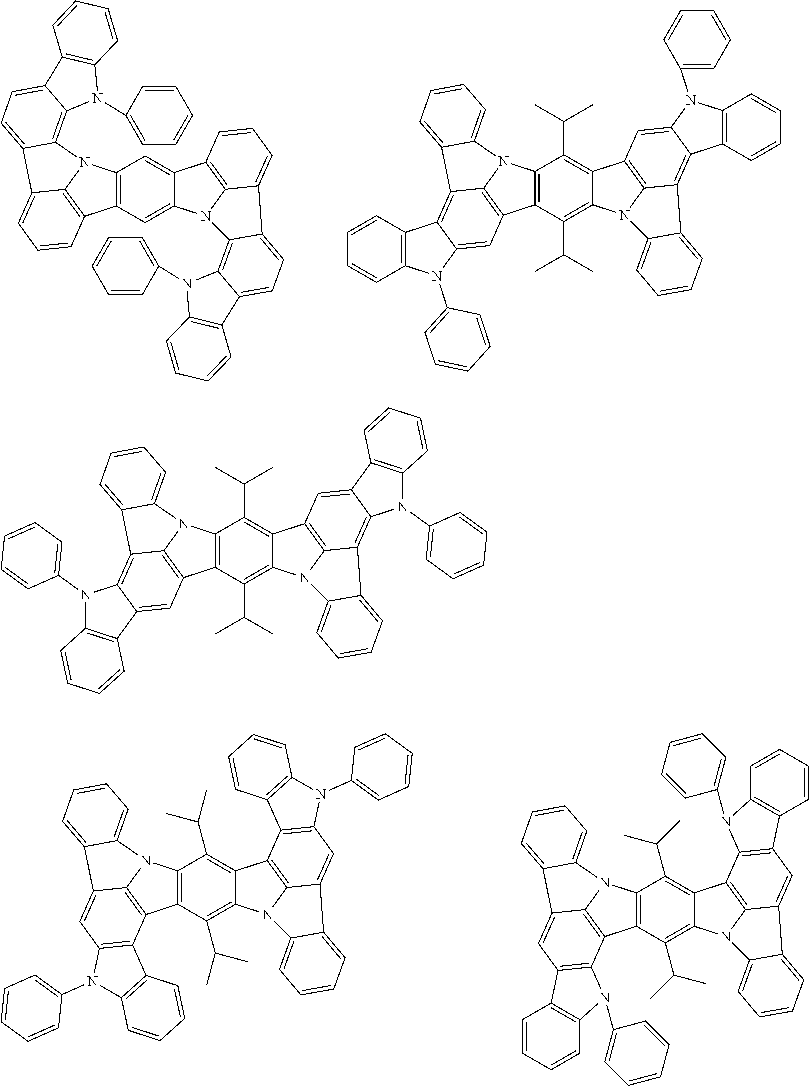

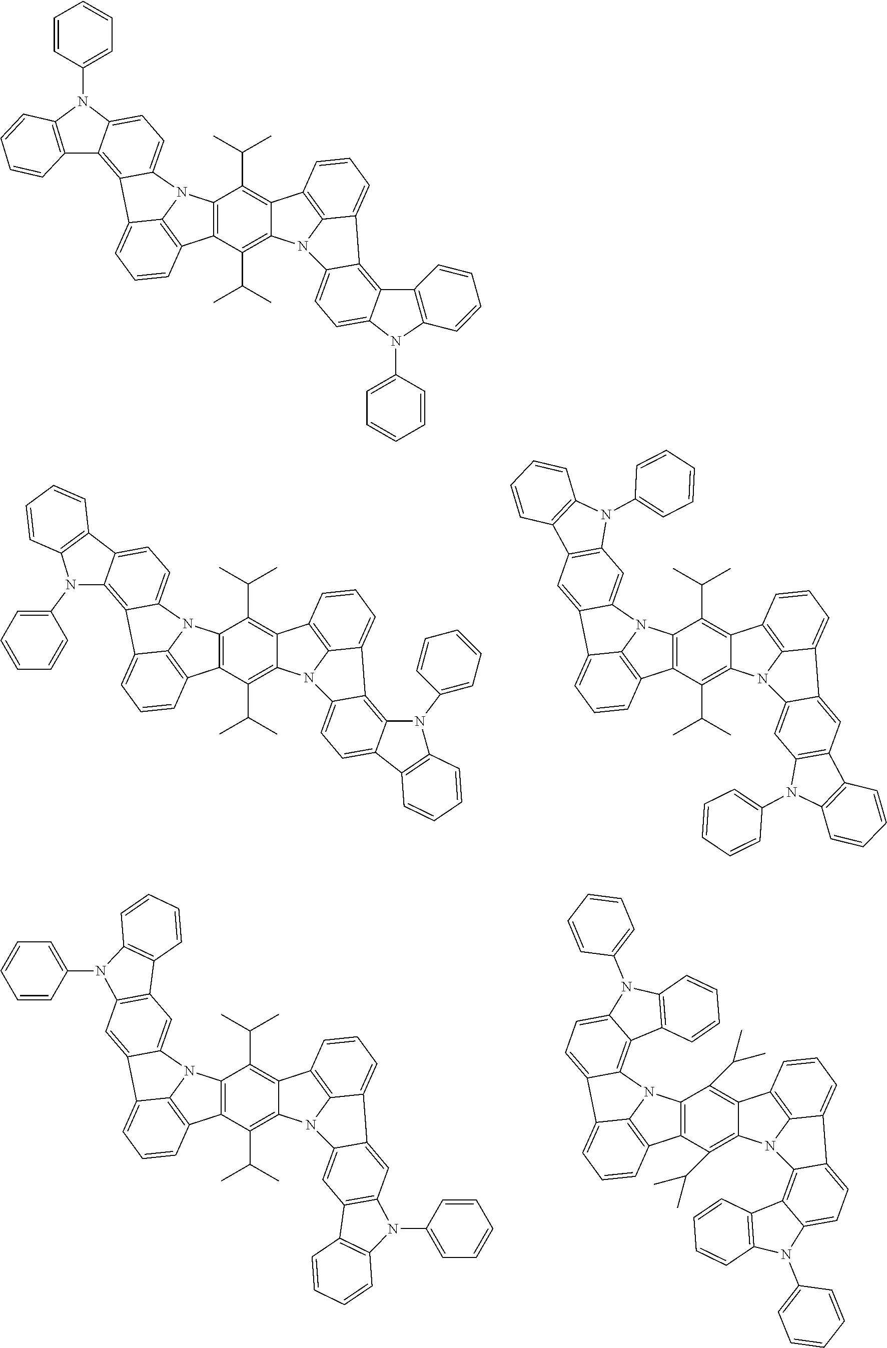

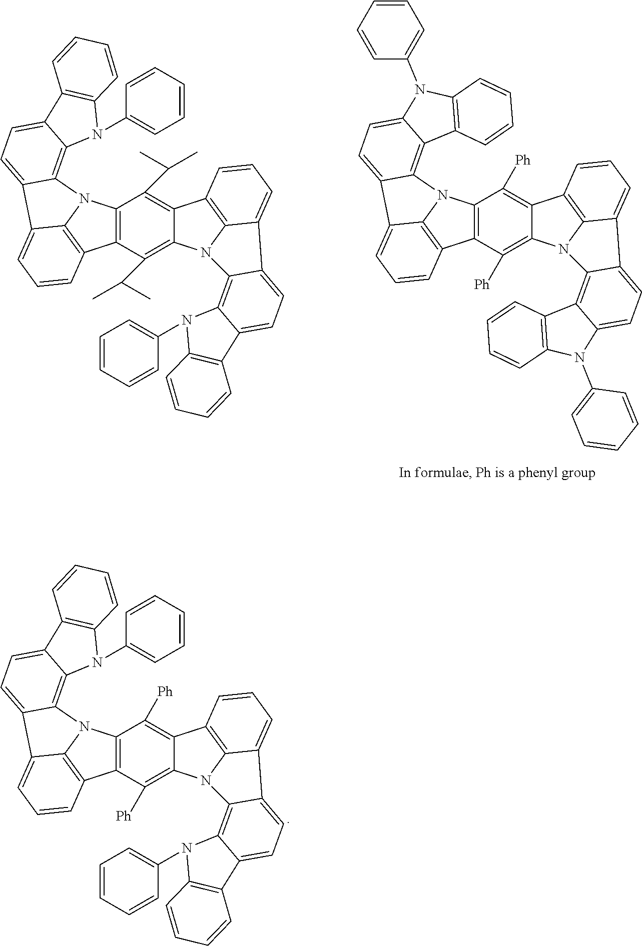 [SMILES: CC(C)c1c2c3cccc4c5ccc6c7ccccc7n(-c7ccccc7)c6c5n(c2c(C(C)C)c2c5cccc6c7ccc8c9ccccc9n(-c9ccccc9)c8c7n(c12)c65)c43 ; c1ccc(-c2c3c4cccc5c6ccc7c(c8ccccc8n7-c7ccccc7)c6n(c3c(-c3ccccc3)c3c6cccc7c8ccc9c(c%10ccccc%10n9-c9ccccc9)c8n(c23)c76)c54)cc1 ; c1ccc(-c2c3c4cccc5c6ccc7c8ccccc8n(-c8ccccc8)c7c6n(c3c(-c3ccccc3)c3c6cccc7c8ccc9c%10ccccc%10n(-c%10ccccc%10)c9c8n(c23)c76)c54)cc1]

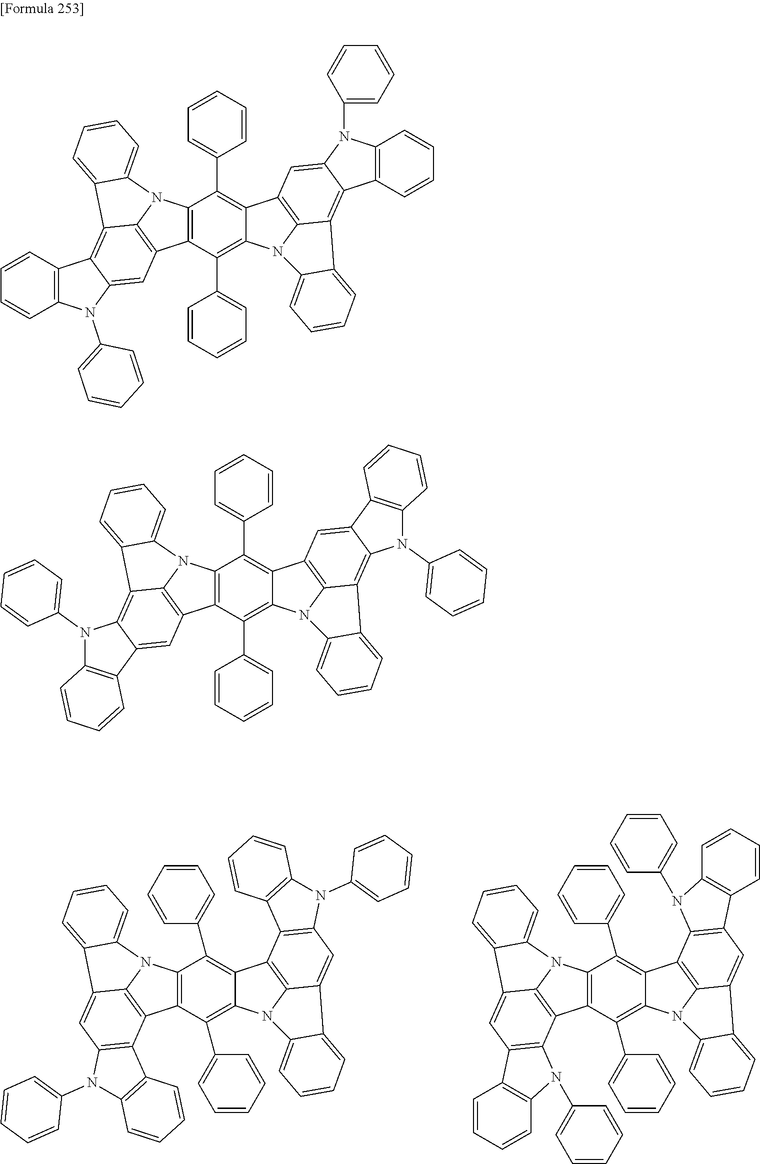 [SMILES: c1ccc(-c2c3c4cc5c(c6ccccc6n5-c5ccccc5)c5c6ccccc6n(c3c(-c3ccccc3)c3c6cc7c(c8ccccc8n7-c7ccccc7)c7c8ccccc8n(c23)c67)c45)cc1 ; c1ccc(-c2c3c4cc5c6ccccc6n(-c6ccccc6)c5c5c6ccccc6n(c3c(-c3ccccc3)c3c6cc7c8ccccc8n(-c8ccccc8)c7c7c8ccccc8n(c23)c67)c45)cc1 ; c1ccc(-c2c3c4c5c6ccccc6n(-c6ccccc6)c5cc5c6ccccc6n(c3c(-c3ccccc3)c3c6c7c8ccccc8n(-c8ccccc8)c7cc7c8ccccc8n(c23)c76)c54)cc1 ; c1ccc(-c2c3c4c5c(cc6c7ccccc7n(c3c(-c3ccccc3)c3c7c8c(cc9c%10ccccc%10n(c23)c97)c2ccccc2n8-c2ccccc2)c64)c2ccccc2n5-c2ccccc2)cc1]

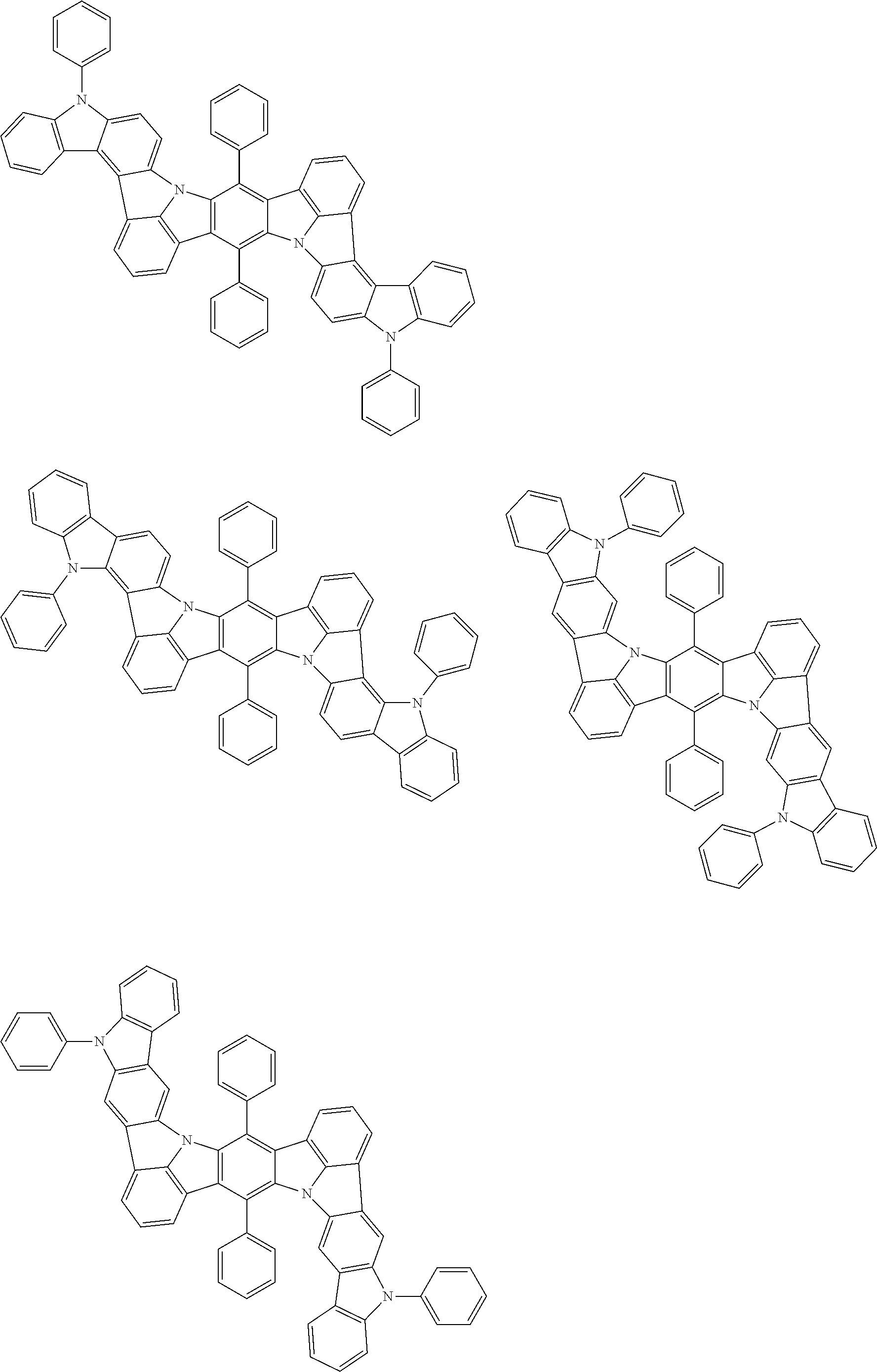

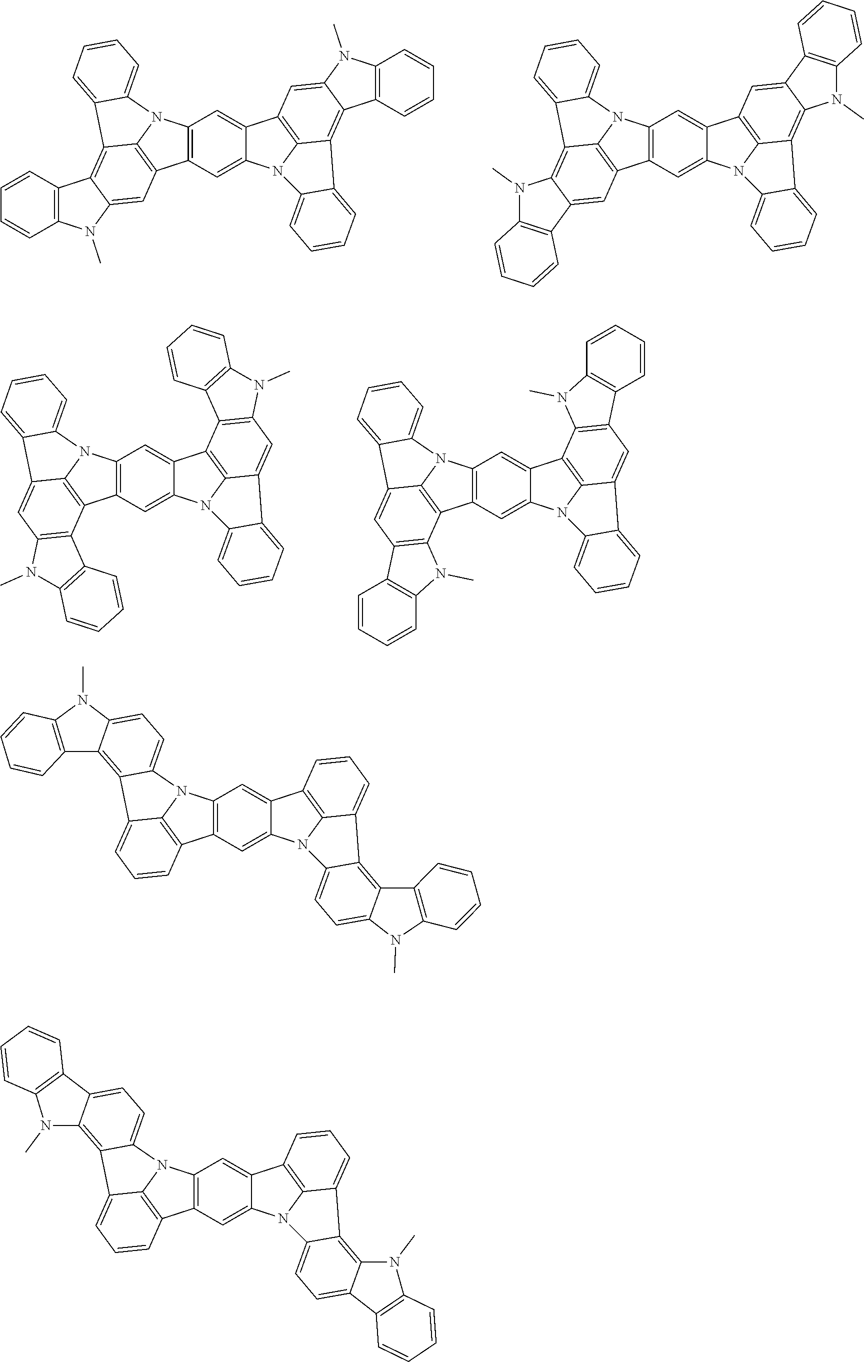

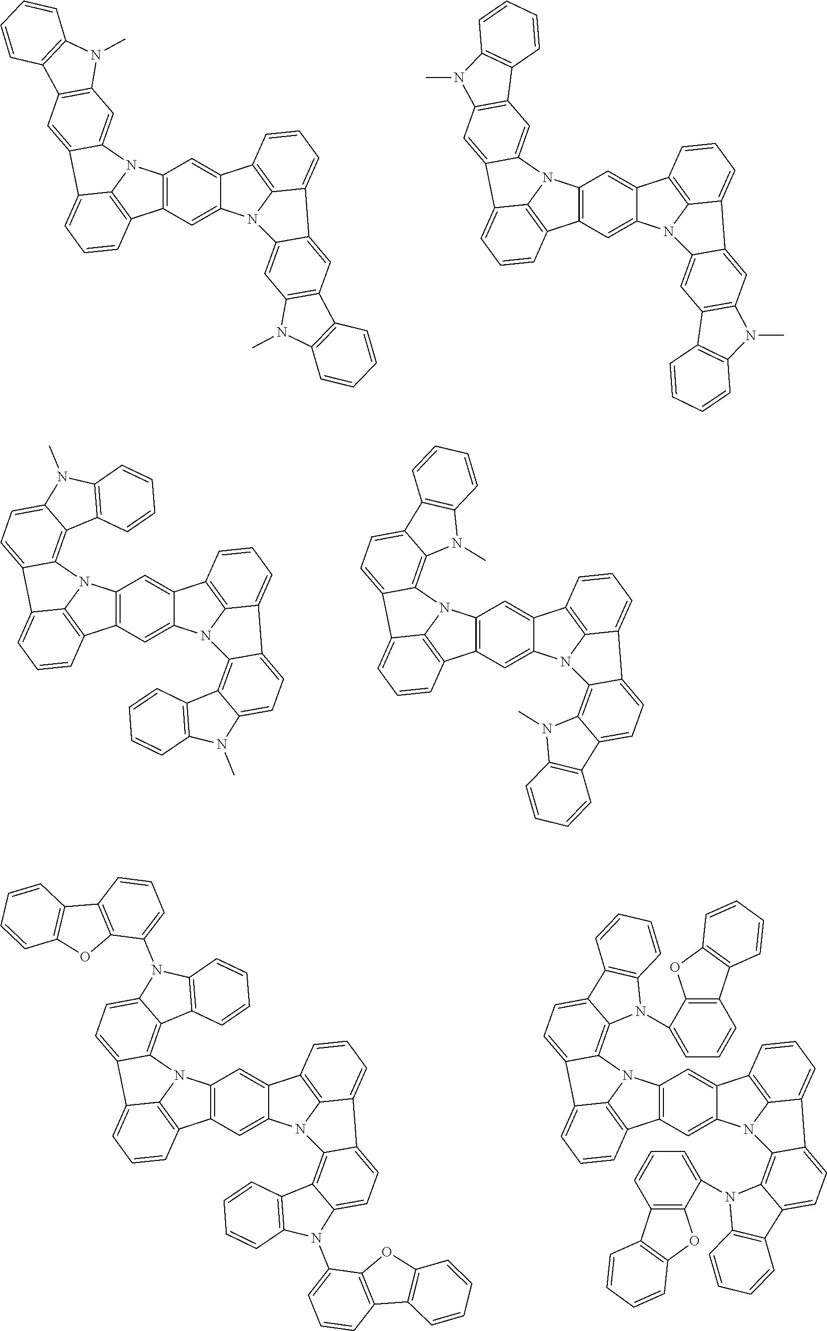

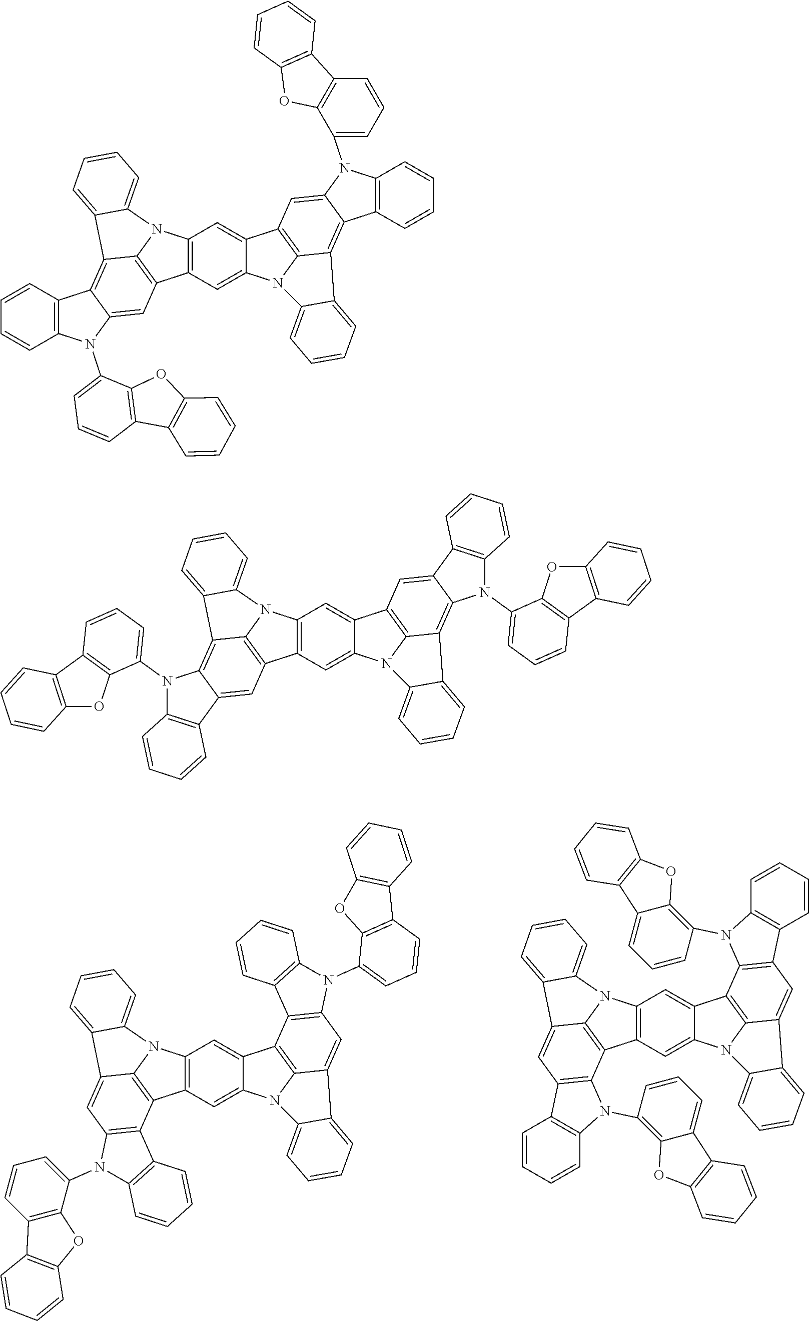

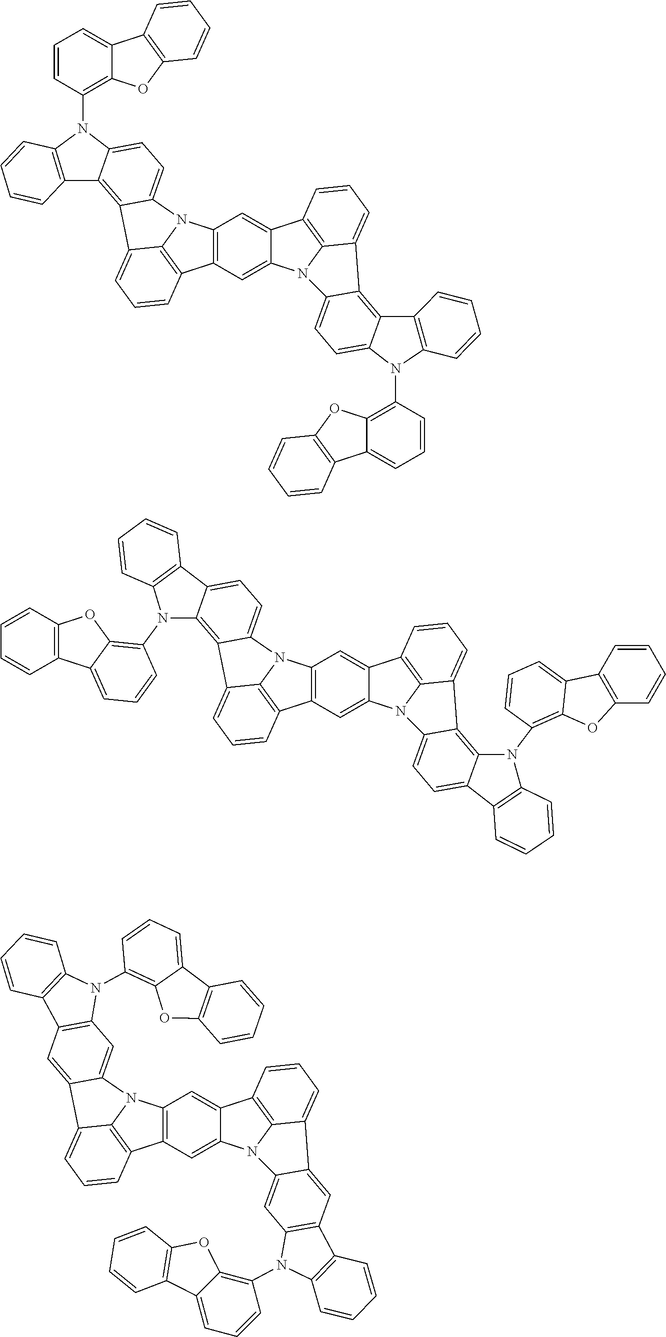 [SMILES: c1ccc2c(c1)oc1c(-n3c4ccccc4c4c5c6cccc7c8cc9c(cc8n(c5ccc43)c76)c3cccc4c5c6c7ccccc7n(-c7cccc8c7oc7ccccc78)c6ccc5n9c34)cccc12 ; c1ccc2c(c1)oc1c(-n3c4ccccc4c4ccc5c(c6cccc7c8cc9c(cc8n5c76)c5cccc6c7c8c(ccc7n9c56)c5ccccc5n8-c5cccc6c5oc5ccccc56)c43)cccc12 ; c1ccc2c(c1)oc1c(-n3c4ccccc4c4cc5c6cccc7c8cc9c(cc8n(c5cc43)c76)c3cccc4c5cc6c7ccccc7n(-c7cccc8c7oc7ccccc78)c6cc5n9c34)cccc12]

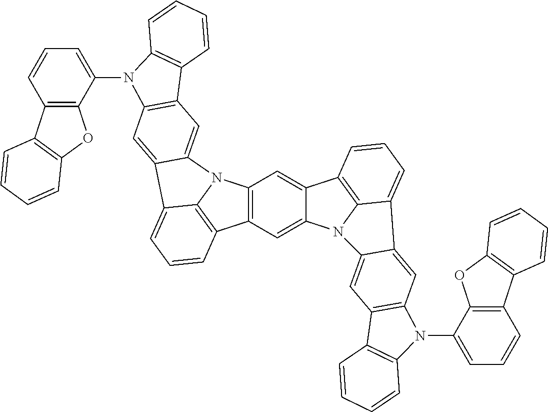 [SMILES: c1ccc2c(c1)oc1c(-n3c4ccccc4c4cc5c(cc43)c3cccc4c6cc7c(cc6n5c34)c3cccc4c5cc6c(cc5n7c43)c3ccccc3n6-c3cccc4c3oc3ccccc34)cccc12]

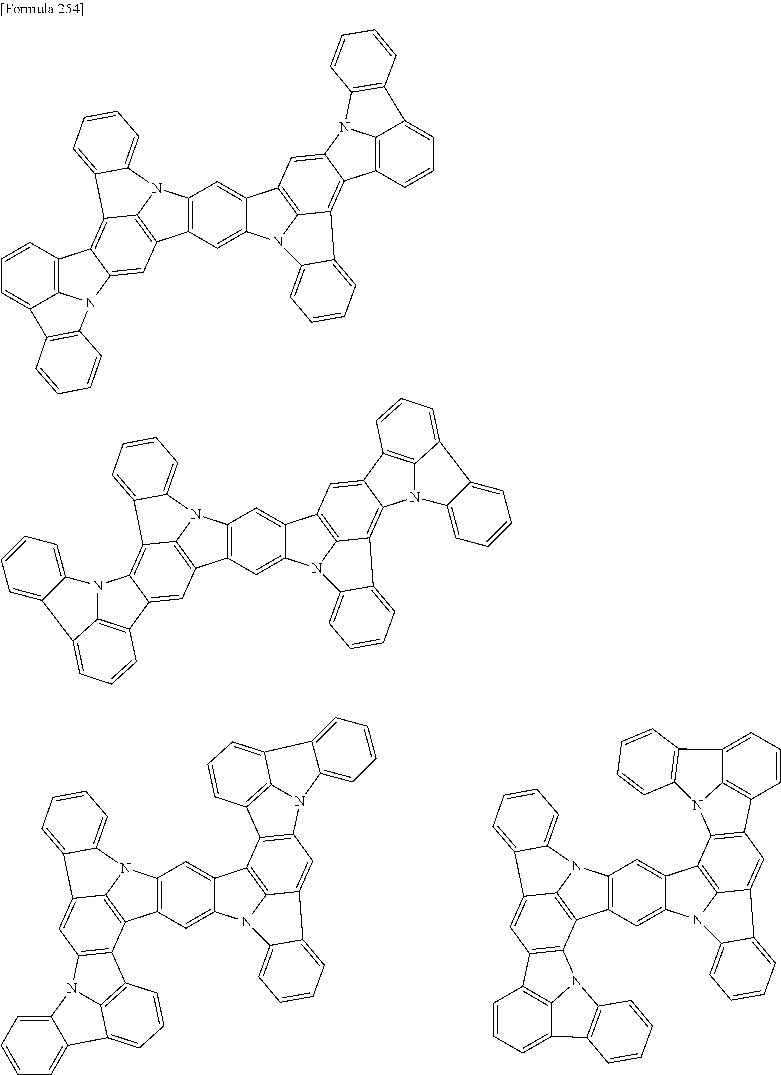 [SMILES: c1ccc2c(c1)c1cccc3c4c5c6ccccc6n6c7cc8c9cc%10c(c%11cccc%12c%13ccccc%13n%10c%12%11)c%10c%11ccccc%11n(c8cc7c(cc4n2c13)c56)c9%10 ; c1ccc2c(c1)c1c3c(cc4c5cccc6c7ccccc7n(c65)c41)c1cc4c(cc1n23)c1cc2c3cccc5c6ccccc6n(c53)c2c2c3ccccc3n4c12 ; c1ccc2c(c1)c1cccc3c4c5c6cc7c(cc6n6c8ccccc8c(cc4n2c13)c56)c1c2c3cccc4c5ccccc5n(c2cc2c5ccccc5n7c21)c43 ; c1ccc2c(c1)c1cc3c4cccc5c6ccccc6n(c54)c3c3c4cc5c(cc4n2c13)c1c2c(cc3c4cccc6c7ccccc7n(c64)c31)c1ccccc1n52]

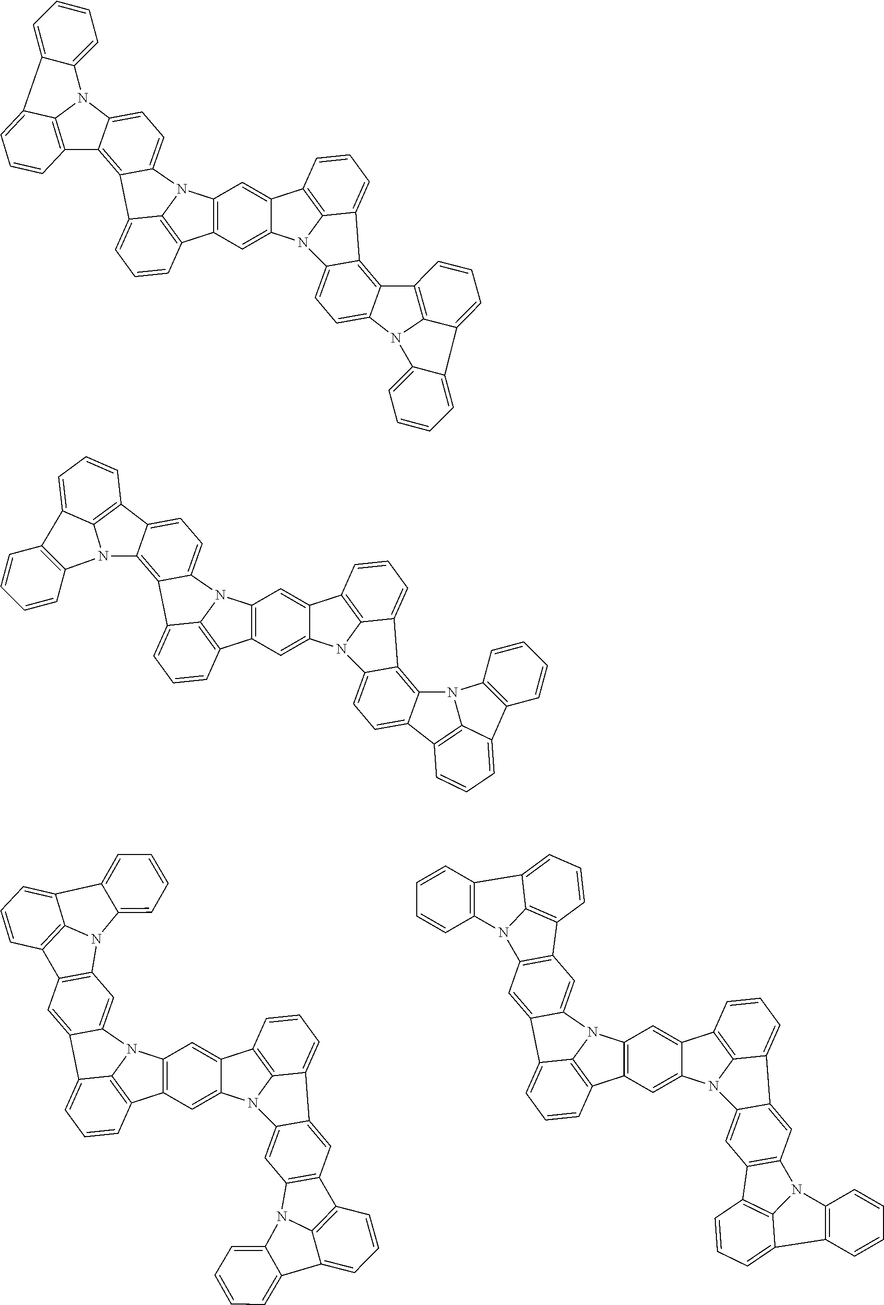 [SMILES: c1ccc2c(c1)c1cccc3c4c5c6cccc7c8cc9c(cc8n(c5ccc4n2c13)c76)c1cccc2c3c4c5cccc6c7ccccc7n(c4ccc3n9c12)c65 ; c1ccc2c(c1)c1cccc3c4ccc5c(c6cccc7c8cc9c(cc8n5c76)c5cccc6c7c(ccc8c%10cccc%11c%12ccccc%12n(c%11%10)c87)n9c56)c4n2c13 ; c1ccc2c(c1)c1cccc3c4cc5c6cccc7c8cc9c(cc8n(c5cc4n2c13)c76)c1cccc2c3cc4c5cccc6c7ccccc7n(c4cc3n9c12)c65 ; c1ccc2c(c1)c1cccc3c4cc5c(cc4n2c13)c1cccc2c3cc4c(cc3n5c12)c1cccc2c3cc5c(cc3n4c21)c1cccc2c3ccccc3n5c21]

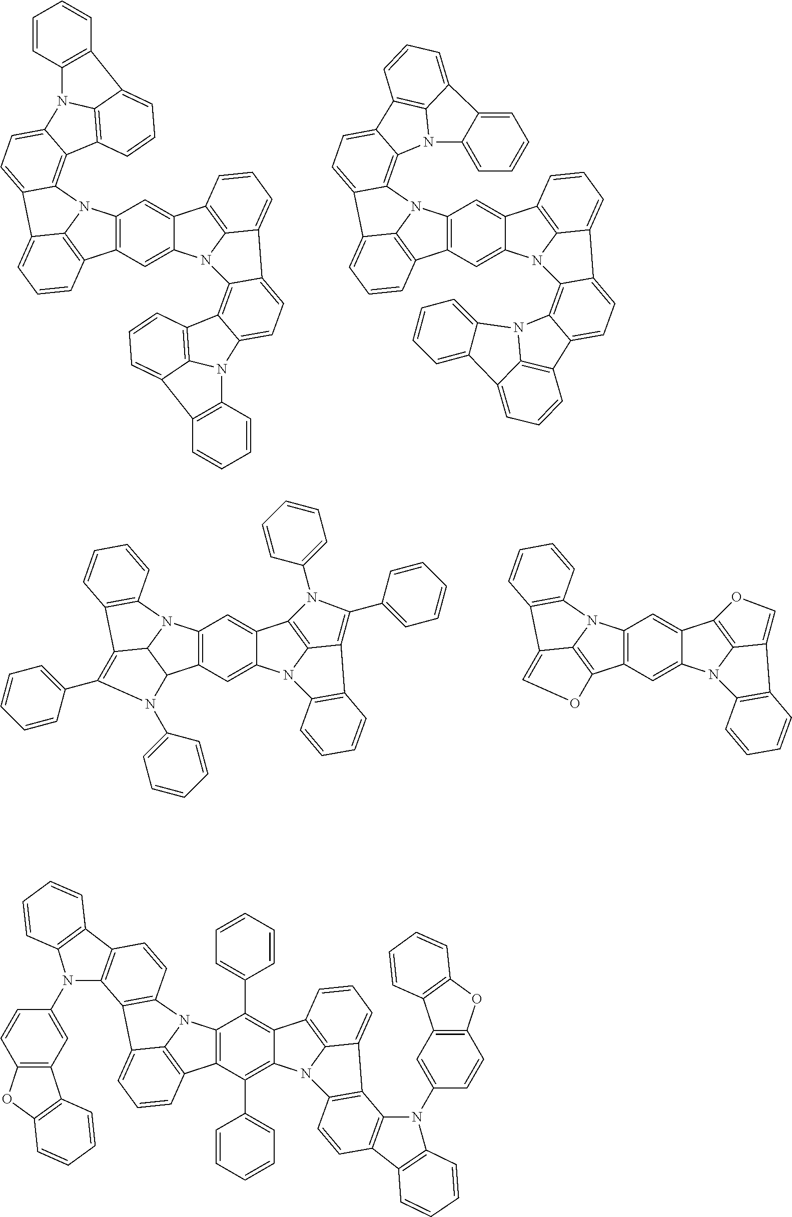

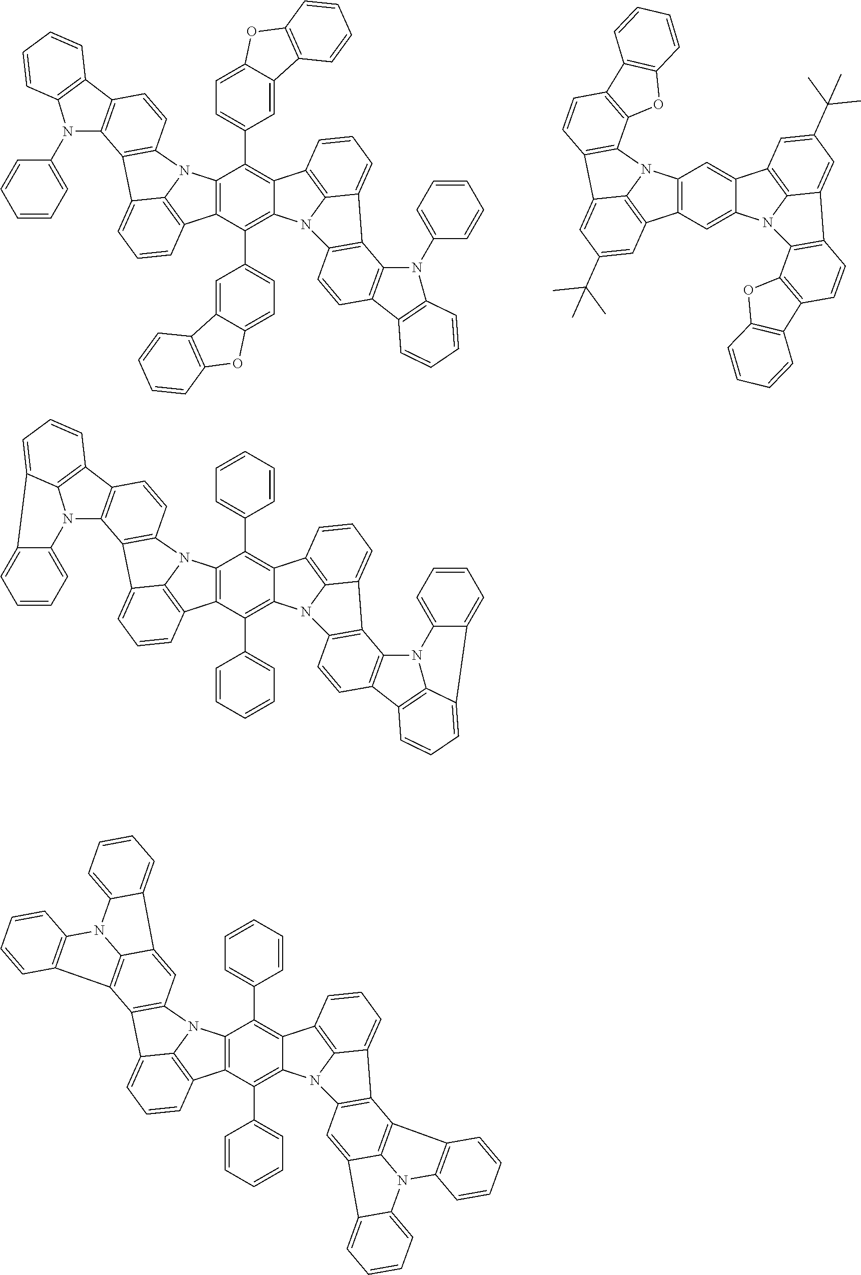

### Compound Represented by Formula (6)

The compound represented by the formula (6) will be described below.

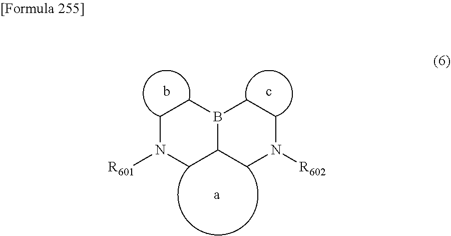

In the formula (6): a ring, b ring and c ring are each independently a substituted or unsubstituted aromatic hydrocarbon ring having 6 to 50 ring carbon atoms or a substituted or unsubstituted heterocycle having 5 to 50 ring atoms;
R₆₀₁ and R₆₀₂ are each independently bonded with the a ring, b ring, or a c ring to form a substituted or unsubstituted heterocycle or does not form a substituted or unsubstituted heterocycle; and
R₆₀₁ and R₆₀₂ not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

The a ring, b ring and c ring are each a ring (a substituted or unsubstituted aromatic hydrocarbon ring having 6 to 50 ring carbon atoms, or a substituted or unsubstituted heterocycle having 5 to 50 ring atoms) fused with the fused bicyclic moiety formed of a boron atom and two nitrogen atoms at the center of the formula (6).

The “aromatic hydrocarbon ring” for the a, b, and c rings has the same structure as the compound formed by introducing a hydrogen atom to the “aryl group” described above.

Ring atoms of the “aromatic hydrocarbon ring” for the a ring include three carbon atoms on the fused bicyclic structure at the center of the formula (6).

Ring atoms of the “aromatic hydrocarbon ring” for the b ring and the c ring include two carbon atoms on a fused bicyclic structure at the center of the formula (6).

Specific examples of the “substituted or unsubstituted aromatic hydrocarbon ring having 6 to 50 ring carbon atoms” include a compound formed by introducing a hydrogen atom to the “aryl group” described in the specific example group G1.

The “heterocycle” for the a, b, and c rings has the same structure as the compound formed by introducing a hydrogen atom to the “heterocyclic group” described above.

Ring atoms of the “heterocycle” for the a ring include three carbon atoms on the fused bicyclic structure at the center of the formula (6). Ring atoms of the “heterocycle” for the b ring and the c ring include two carbon atoms on a fused bicyclic structure at the center of the formula (6). Specific examples of the “substituted or unsubstituted heterocycle having 5 to 50 ring atoms” include a compound formed by introducing a hydrogen atom to the “heterocyclic group” described in the specific example group G2.

R₆₀₁ and R₆₀₂ are optionally each independently bonded with the a ring, b ring, or c ring to form a substituted or unsubstituted heterocycle. The “heterocycle” in this arrangement includes the nitrogen atom on the fused bicyclic structure at the center of the formula (6). The heterocycle in the above arrangement optionally include a hetero atom other than the nitrogen atom. R₆₀₁ and R₆₀₂ bonded with the a ring, b ring, or c ring specifically means that atoms forming R₆₀₁ and R₆₀₂ are bonded with atoms forming the a ring, b ring, or c ring. For instance, R₆₀₁ may be optionally bonded to the a ring to form a bicyclic (or tri-or-more cyclic) fused nitrogen-containing heterocycle, in which the ring including R₆₀₁ and the a ring are fused. Specific examples of the nitrogen-containing heterocycle include a compound corresponding to the nitrogen-containing bi(or-more)cyclic heterocyclic group in the specific example group G2.

The same applies to R₆₀₁ bonded with the b ring, R₆₀₂ bonded with the a ring, and R₆₀₂ bonded with the c ring.

In an exemplary embodiment, the a ring, b ring and c ring in the formula (6) are each independently a substituted or unsubstituted aromatic hydrocarbon ring having 6 to 50 ring carbon atoms.

In an exemplary embodiment, the a ring, b ring and c ring in the formula (6) are each independently a substituted or unsubstituted benzene ring or a substituted or unsubstituted naphthalene ring.

In an exemplary embodiment, R₆₀₁ and R₆₀₂ in the formula (6) are each independently a substituted or unsubstituted aryl group having 6 to 50 ring carbon atoms, or a substituted or unsubstituted heterocyclic group having 5 to 50 ring atoms, preferably a substituted or unsubstituted aryl group having 6 to 50 ring carbon atoms.

In an exemplary embodiment, the compound represented by the formula (6) is represented by a formula (62) below.

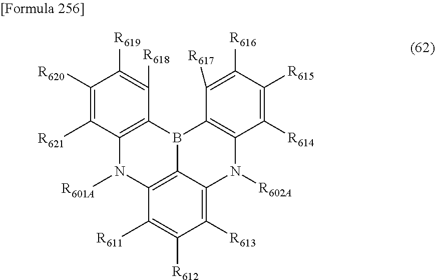

In the formula (62): R_{601A} is bonded with at least one of R₆₁₁ or R₆₂₁ to form a substituted or unsubstituted heterocycle or does not form a substituted or unsubstituted heterocycle;
R_{602A} is bonded with at least one of R₆₁₃ or R₆₁₄A to form a substituted or unsubstituted heterocycle or does not form a substituted or unsubstituted heterocycle;
R_{601A} and R_{602A} not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
at least one combination of adjacent two or more of R₆₁₁ to R₆₂₁ may be mutually bonded to form a substituted or unsubstituted monocyclic ring, mutually bonded to form a substituted or unsubstituted fused ring, or not mutually bonded; and
R₆₁₁ to R₆₂₁ not forming the substituted or unsubstituted heterocycle, not forming the monocyclic ring and not forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

R_{601A} and R_{602A} in the formula (62) are groups corresponding to R₆₀₁ and R₆₀₂ in the formula (6), respectively.

For instance, R_{601A} and R₆₁₁ are optionally bonded with each other to form a bicyclic (or tri-or-more cyclic) nitrogen-containing heterocycle, in which the ring including R_{601A} and R₆₁₁ and a benzene ring corresponding to the a ring are fused. Specific examples of the nitrogen-containing heterocycle include a compound corresponding to the nitrogen-containing bi(or-more)cyclic heterocyclic group in the specific example group G2. The same applies to R_{601A} bonded with R₆₂₁, R_{602A} bonded with R₆₁₃, and R_{602A} bonded with R₆₁₄.

At least one combination of adjacent two or more of R₆₁₁ to R₆₂₁ may be mutually bonded to form a substituted or unsubstituted monocyclic ring, or mutually bonded to form a substituted or unsubstituted monocyclic ring.

For instance, R₆₁₁ and R₆₁₂ are mutually bonded to form a structure in which a benzene ring, indole ring, pyrrole ring, benzofuran ring, benzothiophene ring or the like is bonded to the six-membered ring bonded with R₆₁₁ and R₆₁₂, the resultant fused ring forming a naphthalene ring, carbazole ring, indole ring, dibenzofuran ring, or dibenzothiophene ring, respectively.

In an exemplary embodiment, R₆₁₁ to R₆₂₁ not contributing to ring formation are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, R₆₁₁ to R₆₂₁ not contributing to ring formation are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₆₁₁ to R₆₂₁ not contributing to ring formation are each independently a hydrogen atom, or a substituted or unsubstituted alkyl group having 1 to 50 carbon atoms.

In an exemplary embodiment, R₆₁₁ to R₆₂₁ not contributing to ring formation are each independently a hydrogen atom, or a substituted or unsubstituted alkyl group having 1 to 50 carbon atoms, and
at least one of R₆₁₁ to R₆₂₁ is a substituted or unsubstituted alkyl group having 1 to 50 carbon atoms.

In an exemplary embodiment, the compound represented by the formula (62) is represented by a formula (63) below.

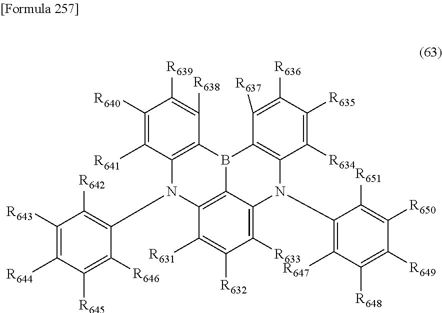

In the formula (63): R₆₃₁ is bonded with R₆₄₆ to form a substituted or unsubstituted heterocycle or does not form a substituted or unsubstituted heterocycle;
R₆₃₃ is bonded with R₆₄₇ to form a substituted or unsubstituted heterocycle or does not form a substituted or unsubstituted heterocycle;
R₆₃₄ is bonded with R₆₅₁ to form a substituted or unsubstituted heterocycle or does not form a substituted or unsubstituted heterocycle;
R₆₄₁ is bonded with R₆₄₂ to form a substituted or unsubstituted heterocycle or does not form a substituted or unsubstituted heterocycle;
at least one combination of adjacent two or more of R₆₃₁ to R₆₅₁ are mutually bonded to form a substituted or unsubstituted monocyclic ring, mutually bonded to form a substituted or unsubstituted fused ring, or not mutually bonded; and
R₆₃₁ to R₆₅₁ not forming the substituted or unsubstituted heterocycle, not forming the monocyclic ring and not forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

R₆₃₁ are optionally mutually bonded with R₆₄₆ to form a substituted or unsubstituted heterocycle. For instance, R₆₃₁ and R₆₄₆ are optionally bonded with each other to form a tri-or-more cyclic nitrogen-containing heterocycle, in which a benzene ring bonded with R₆₄₆, a ring including a nitrogen atom, and a benzene ring corresponding to the a ring are fused. Specific examples of the nitrogen-containing heterocycle include a compound corresponding to the nitrogen-containing tri(-or-more)cyclic heterocyclic group in the specific example group G2. The same applies to R₆₃₃ bonded with R₆₄₇, R₆₃₄ bonded with R₆₅₁, and R₆₄₁ bonded with R₆₄₂.

In an exemplary embodiment, R₆₃₁ to R₆₅₁, which do not contribute to ring formation, are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, R₆₃₁ to R₆₅₁, which do not contribute to ring formation, are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₆₃₁ to R₆₅₁, which do not contribute to ring formation, are each independently a hydrogen atom, or a substituted or unsubstituted alkyl group having 1 to 50 carbon atoms.

In an exemplary embodiment, R₆₃₁ to R₆₅₁, which do not contribute to ring formation, are each independently a hydrogen atom, or a substituted or unsubstituted alkyl group having 1 to 50 carbon atoms, and
at least one of R₆₃₁ to R₆₅₁ is a substituted or unsubstituted alkyl group having 1 to 50 carbon atoms.

In an exemplary embodiment, the compound represented by the formula (63) is represented by a formula (63A) below.

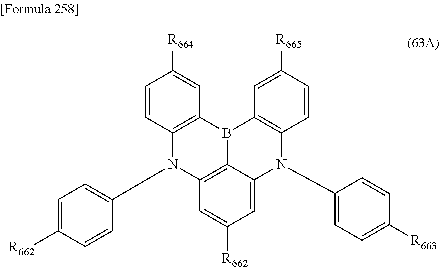

In the formula (63A): R₆₆₁ is a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms; and
R₆₆₂ to R₆₆₅ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary embodiment, R₆₆₁ to R₆₆₅, which do not contribute to ring formation, are each independently a substituted or unsubstituted alkyl group having 1 to 50 carbon atoms, or a substituted or unsubstituted aryl group having 6 to 50 ring carbon atoms.

In an exemplary embodiment, R₆₆₁ to R₆₆₅ are each independently a substituted or unsubstituted alkyl group having 1 to 50 carbon atoms.

In an exemplary embodiment, the compound represented by the formula (63) is represented by a formula (63B) below.

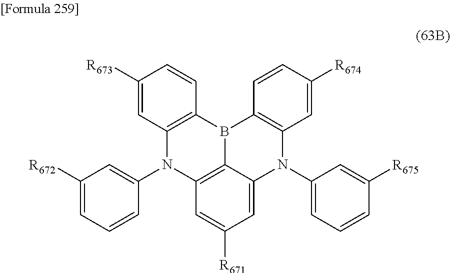

In the formula (63B): R₆₇₁ and R₆₇₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms; and
R₆₇₃ to R₆₇₅ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms.

In an exemplary embodiment, the compound represented by the formula (63) is represented by a formula (63B′) below.

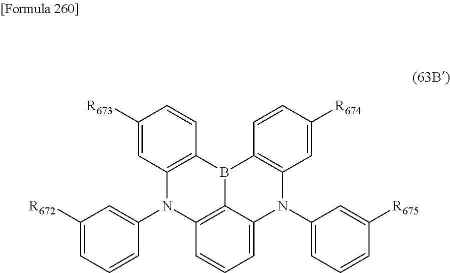

In the formula (63B′), R₆₇₂ to R₆₇₅ each independently represent the same as R₆₇₂ to R₆₇₅ in the formula (63B).

In an exemplary embodiment, at least one of R₆₇₁ to R₆₇₅ is: a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms; and

In an exemplary embodiment: R₆₇₂ is a hydrogen atom, a substituted or unsubstituted alkyl group having 1 to 50 carbon atoms, a group represented by —N(R₉₀₆)(R₉₀₇), or a substituted or unsubstituted aryl group having 6 to 50 ring carbon atoms; and
R₆₇₁, and R₆₇₃ to R₆₇₅ are each independently a substituted or unsubstituted alkyl group having 1 to 50 carbon atoms, a group represented by —N(R₉₀₆)(R₉₀₇), or a substituted or unsubstituted aryl group having 6 to 50 ring carbon atoms.

In an exemplary embodiment, the compound represented by the formula (63) is represented by a formula (63C) below.

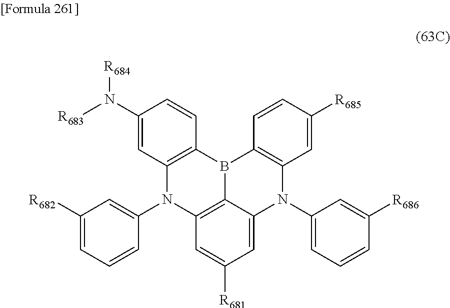

In the formula (63C): R₆₈₁ and R₆₈₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms; and
R₆₈₃ to R₆₈₆ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary embodiment, the compound represented by the formula (63) is represented by a formula (63C′) below.

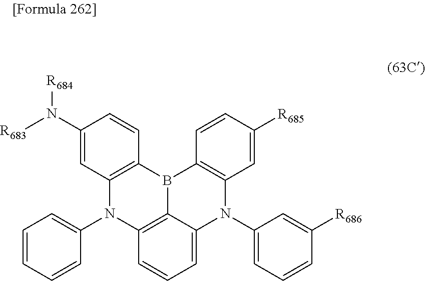

In the formula (63C′), R₆₈₃ to R₆₈₆ each independently represent the same as R₆₈₃ to R₆₈₆ in the formula (63C).

In an exemplary embodiment, R₆₈₁ to R₆₈₆, which do not contribute to ring formation, are each independently a substituted or unsubstituted alkyl group having 1 to 50 carbon atoms, or a substituted or unsubstituted aryl group having 6 to 50 ring carbon atoms.

In an exemplary embodiment, R₆₈₁ to R₆₈₆ are each independently a substituted or unsubstituted aryl group having 6 to 50 ring carbon atoms.

The compound represented by the formula (6) is producible by initially bonding the a ring, b ring and c ring with linking groups (a group including N—R₆₀₁ and a group including N—R₆₀₂) to form an intermediate (first reaction), and bonding the a ring, b ring and c ring with a linking group (a group including a boron atom) to form a final product (second reaction). In the first reaction, an amination reaction (e.g. Buchwald-Hartwig reaction) is applicable. In the second reaction, Tandem Hetero-Friedel-Crafts Reactions or the like is applicable.

### Specific Examples of Compound Represented by Formula (6)

Specific examples of the compound represented by the formula (6) are shown below. It should however be noted that these specific examples are merely exemplary and do not limit the compound represented by the formula (6).

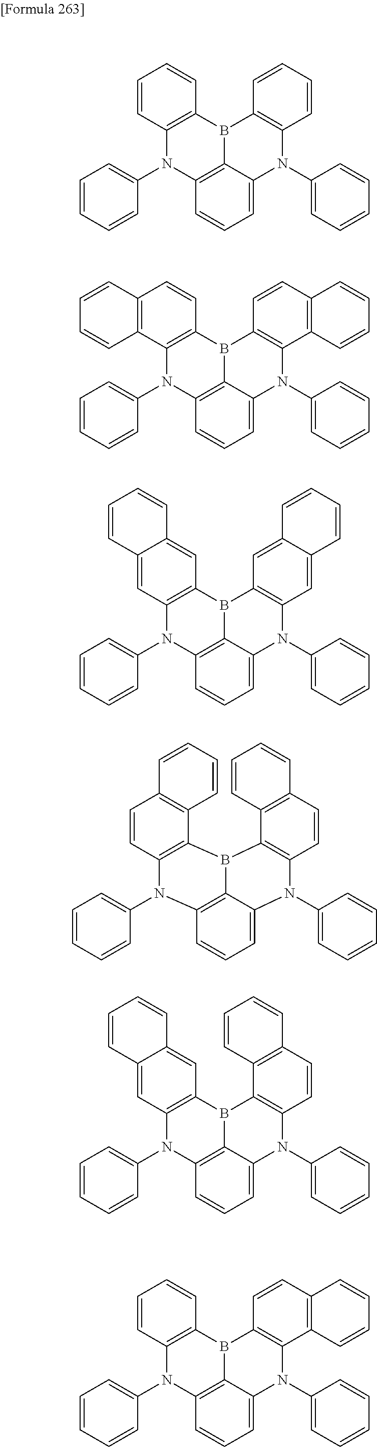

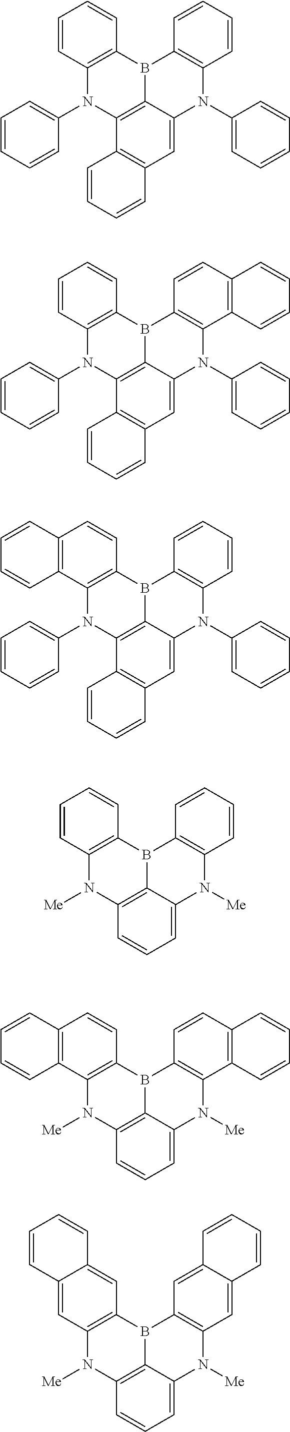 [SMILES: c1ccc(N2c3ccccc3B3c4ccccc4N(c4ccccc4)c4c3c2cc2ccccc42)cc1 ; c1ccc(N2c3cc4ccccc4c4c3B(c3ccccc3N4c3ccccc3)c3ccc4ccccc4c32)cc1 ; c1ccc(N2c3ccccc3B3c4ccc5ccccc5c4N(c4ccccc4)c4c3c2cc2ccccc42)cc1 ; CN1c2ccccc2B2c3ccccc3N(C)c3cccc1c32 ; CN1c2cccc3c2B(c2ccc4ccccc4c21)c1ccc2ccccc2c1N3C ; CN1c2cc3ccccc3cc2B2c3cc4ccccc4cc3N(C)c3cccc1c32]

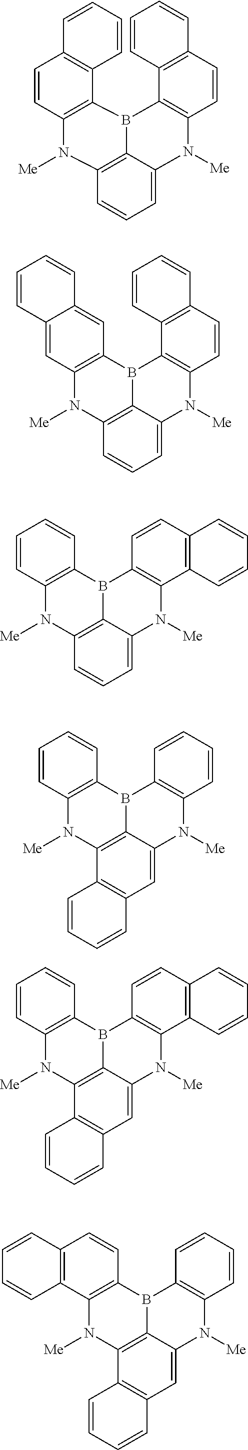

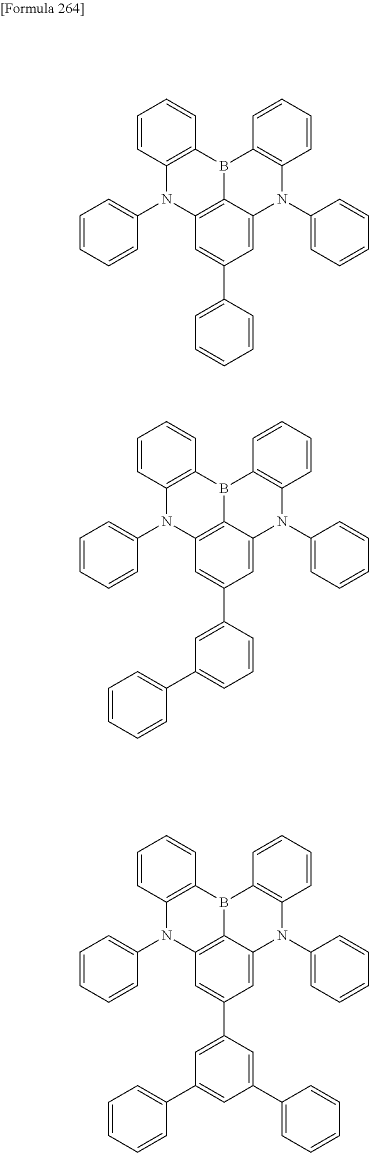 [SMILES: c1ccc(-c2cc3c4c(c2)N(c2ccccc2)c2ccccc2B4c2ccccc2N3c2ccccc2)cc1 ; c1ccc(-c2cccc(-c3cc4c5c(c3)N(c3ccccc3)c3ccccc3B5c3ccccc3N4c3ccccc3)c2)cc1 ; c1ccc(-c2cc(-c3ccccc3)cc(-c3cc4c5c(c3)N(c3ccccc3)c3ccccc3B5c3ccccc3N4c3ccccc3)c2)cc1]

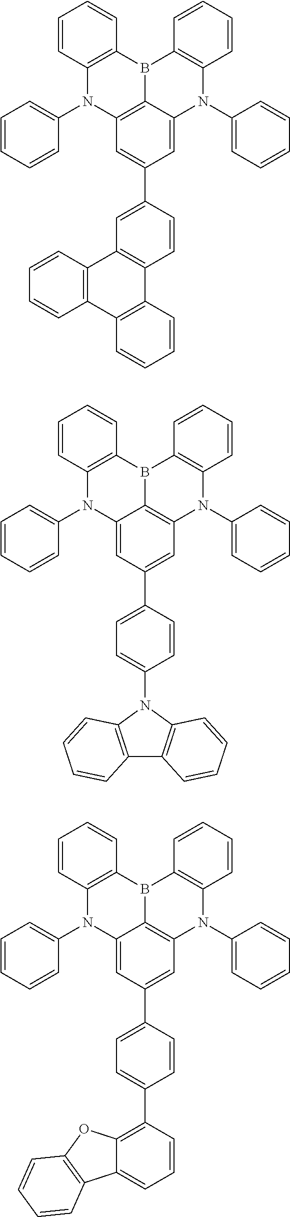

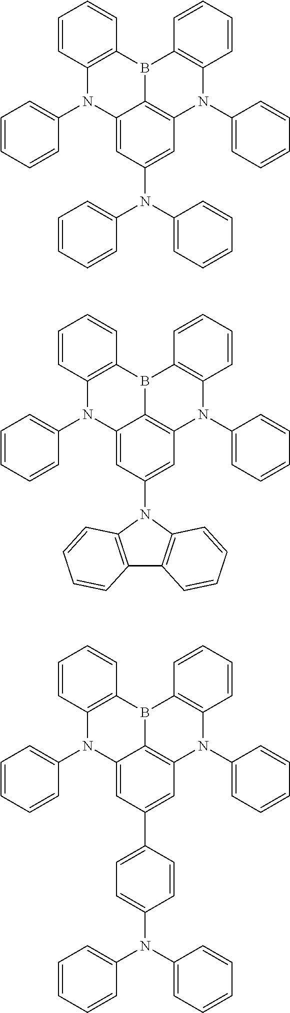

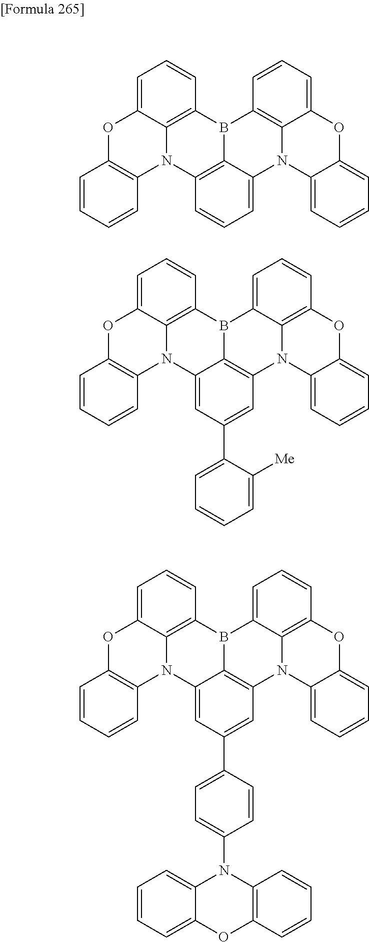 [SMILES: c1ccc2c(c1)Oc1cccc3c1N2c1cccc2c1B3c1cccc3c1N2c1ccccc1O3 ; Cc1ccccc1-c1cc2c3c(c1)N1c4ccccc4Oc4cccc(c41)B3c1cccc3c1N2c1ccccc1O3 ; c1ccc2c(c1)Oc1ccccc1N2c1ccc(-c2cc3c4c(c2)N2c5ccccc5Oc5cccc(c52)B4c2cccc4c2N3c2ccccc2O4)cc1]

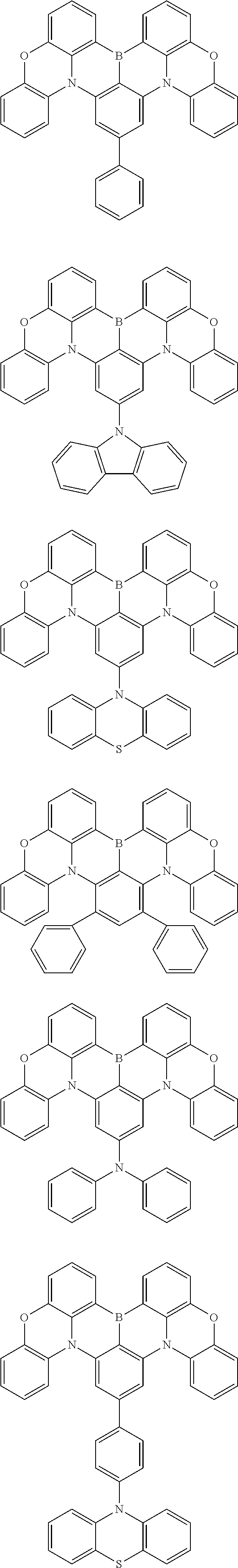

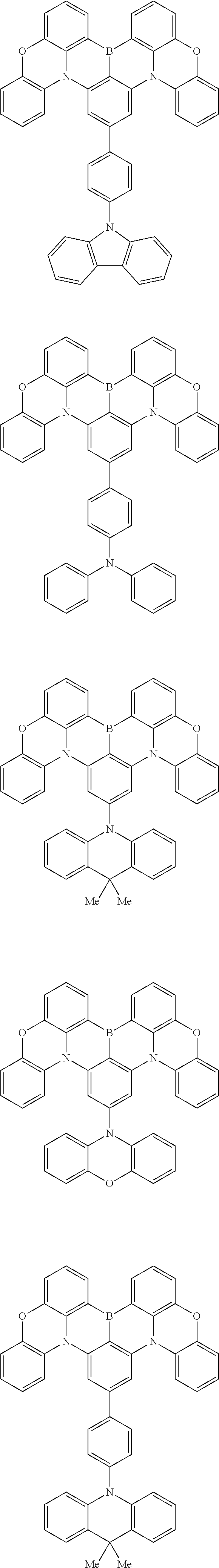 [SMILES: c1ccc2c(c1)Oc1cccc3c1N2c1cc(-c2ccc(-n4c5ccccc5c5ccccc54)cc2)cc2c1B3c1cccc3c1N2c1ccccc1O3 ; c1ccc(N(c2ccccc2)c2ccc(-c3cc4c5c(c3)N3c6ccccc6Oc6cccc(c63)B5c3cccc5c3N4c3ccccc3O5)cc2)cc1 ; CC1(C)c2ccccc2N(c2cc3c4c(c2)N2c5ccccc5Oc5cccc(c52)B4c2cccc4c2N3c2ccccc2O4)c2ccccc21 ; c1ccc2c(c1)Oc1ccccc1N2c1cc2c3c(c1)N1c4ccccc4Oc4cccc(c41)B3c1cccc3c1N2c1ccccc1O3 ; CC1(C)c2ccccc2N(c2ccc(-c3cc4c5c(c3)N3c6ccccc6Oc6cccc(c63)B5c3cccc5c3N4c3ccccc3O5)cc2)c2ccccc21]

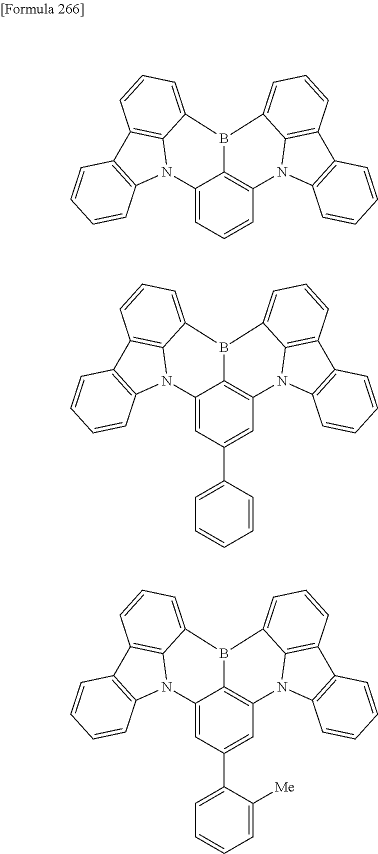 [SMILES: c1cc2c3c(c1)-n1c4ccccc4c4cccc(c41)B3c1cccc3c4ccccc4n-2c13 ; c1ccc(-c2cc3c4c(c2)-n2c5ccccc5c5cccc(c52)B4c2cccc4c5ccccc5n-3c24)cc1 ; Cc1ccccc1-c1cc2c3c(c1)-n1c4ccccc4c4cccc(c41)B3c1cccc3c4ccccc4n-2c13]

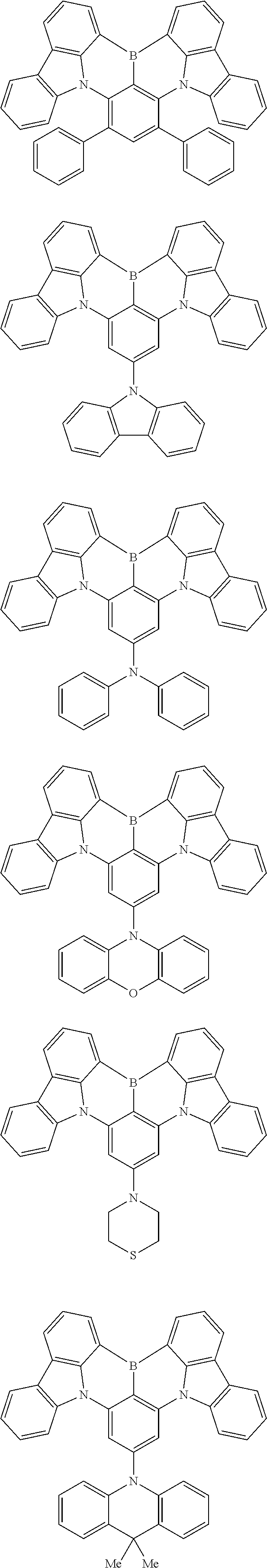

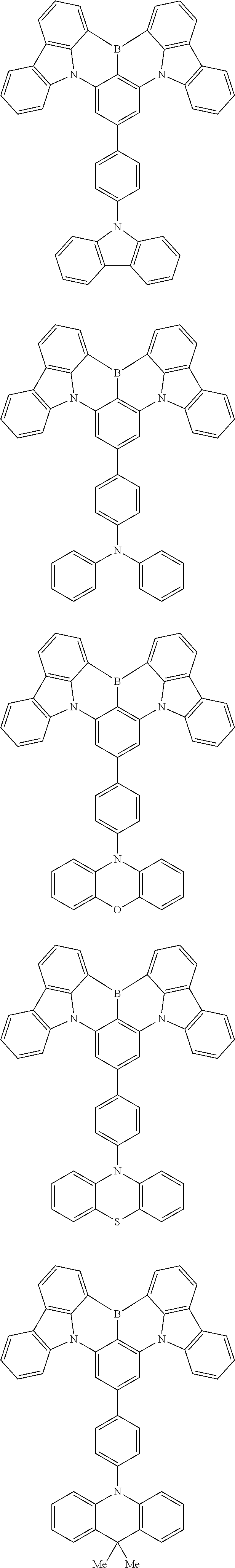 [SMILES: c1ccc2c(c1)c1ccccc1n2-c1ccc(-c2cc3c4c(c2)-n2c5ccccc5c5cccc(c52)B4c2cccc4c5ccccc5n-3c24)cc1 ; c1ccc(N(c2ccccc2)c2ccc(-c3cc4c5c(c3)-n3c6ccccc6c6cccc(c63)B5c3cccc5c6ccccc6n-4c35)cc2)cc1 ; c1ccc2c(c1)Oc1ccccc1N2c1ccc(-c2cc3c4c(c2)-n2c5ccccc5c5cccc(c52)B4c2cccc4c5ccccc5n-3c24)cc1 ; c1ccc2c(c1)Sc1ccccc1N2c1ccc(-c2cc3c4c(c2)-n2c5ccccc5c5cccc(c52)B4c2cccc4c5ccccc5n-3c24)cc1 ; CC1(C)c2ccccc2N(c2ccc(-c3cc4c5c(c3)-n3c6ccccc6c6cccc(c63)B5c3cccc5c6ccccc6n-4c35)cc2)c2ccccc21]

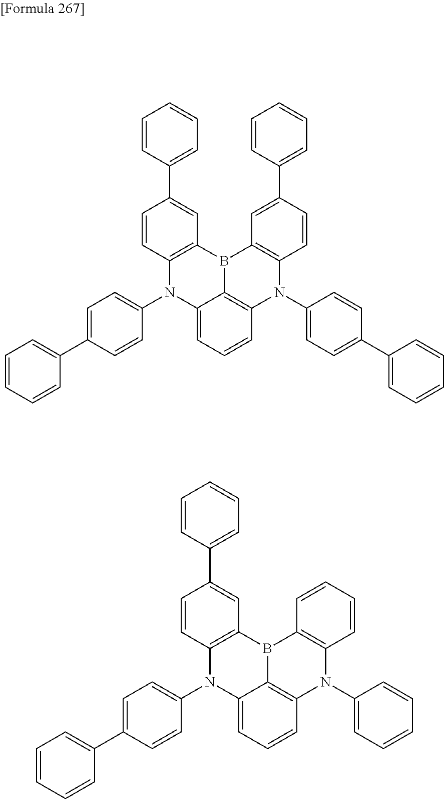 [SMILES: c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5ccccc5N(c5ccccc5)c5cccc3c54)cc2)cc1]

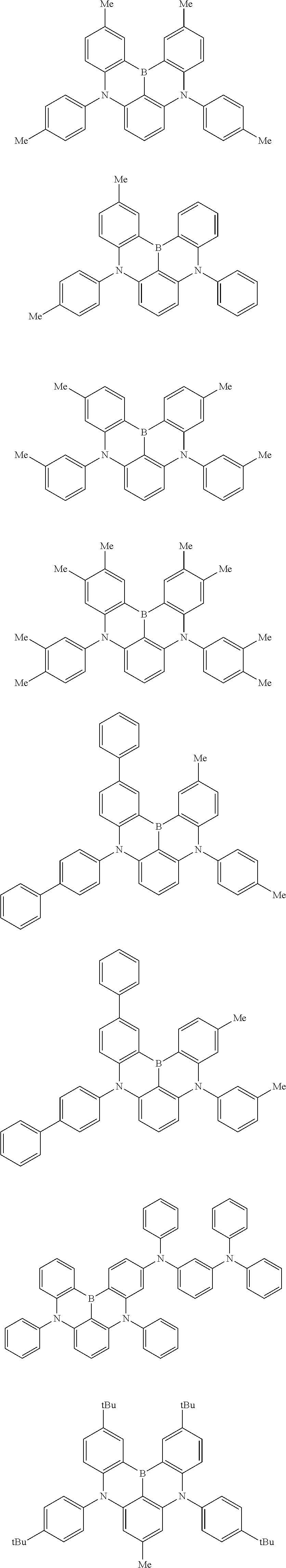

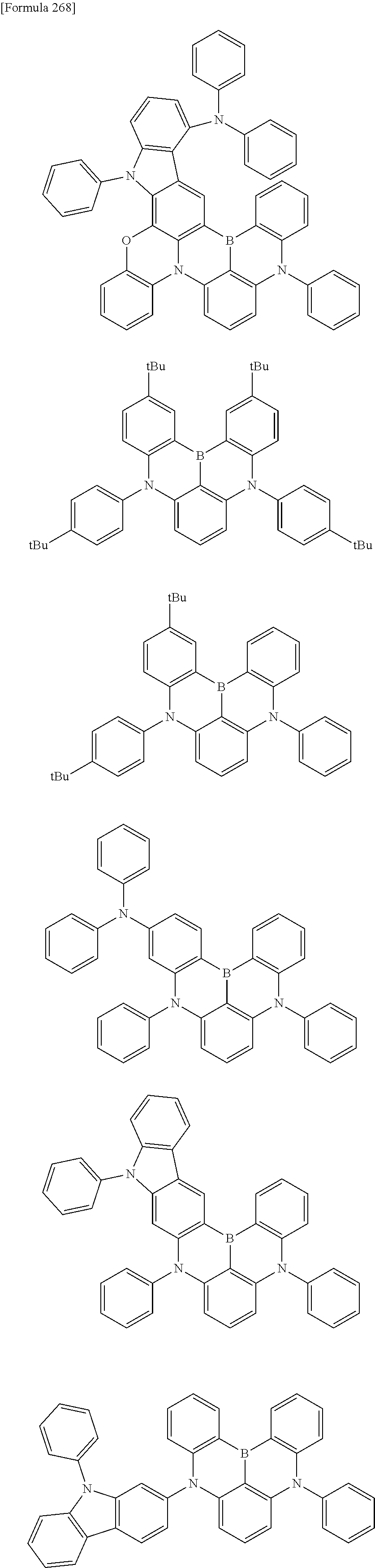

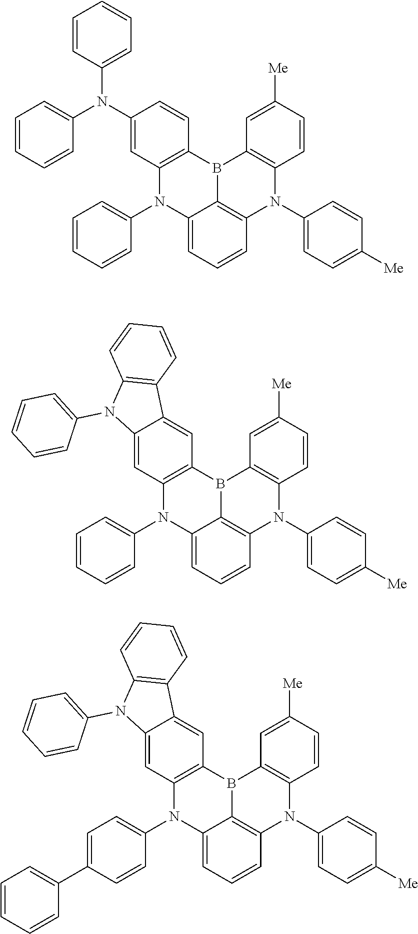 [SMILES: Cc1ccc(N2c3ccc(C)cc3B3c4ccc(N(c5ccccc5)c5ccccc5)cc4N(c4ccccc4)c4cccc2c43)cc1 ; Cc1ccc(N2c3ccc(C)cc3B3c4cc5c6ccccc6n(-c6ccccc6)c5cc4N(c4ccccc4)c4cccc2c43)cc1 ; Cc1ccc(N2c3ccc(C)cc3B3c4cc5c6ccccc6n(-c6ccccc6)c5cc4N(c4ccc(-c5ccccc5)cc4)c4cccc2c43)cc1]

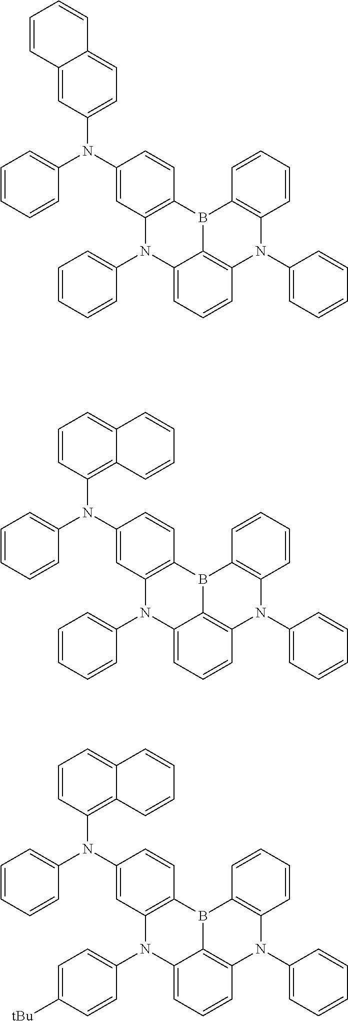 [SMILES: c1ccc(N(c2ccc3c(c2)N(c2ccccc2)c2cccc4c2B3c2ccccc2N4c2ccccc2)c2ccc3ccccc3c2)cc1 ; c1ccc(N2c3ccccc3B3c4ccc(N(c5ccccc5)c5cccc6ccccc56)cc4N(c4ccccc4)c4cccc2c43)cc1 ; CC(C)(C)c1ccc(N2c3cc(N(c4ccccc4)c4cccc5ccccc45)ccc3B3c4ccccc4N(c4ccccc4)c4cccc2c43)cc1]

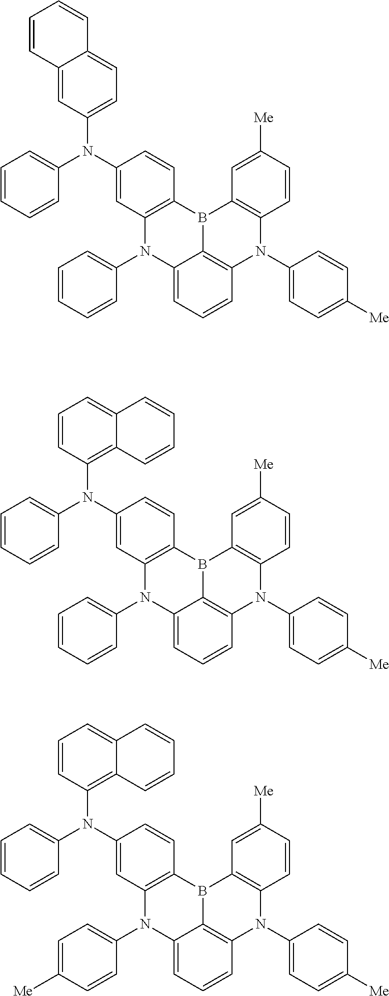 [SMILES: Cc1ccc(N2c3ccc(C)cc3B3c4ccc(N(c5ccccc5)c5ccc6ccccc6c5)cc4N(c4ccccc4)c4cccc2c43)cc1 ; Cc1ccc(N2c3ccc(C)cc3B3c4ccc(N(c5ccccc5)c5cccc6ccccc56)cc4N(c4ccccc4)c4cccc2c43)cc1 ; Cc1ccc(N2c3ccc(C)cc3B3c4ccc(N(c5ccccc5)c5cccc6ccccc56)cc4N(c4ccc(C)cc4)c4cccc2c43)cc1]

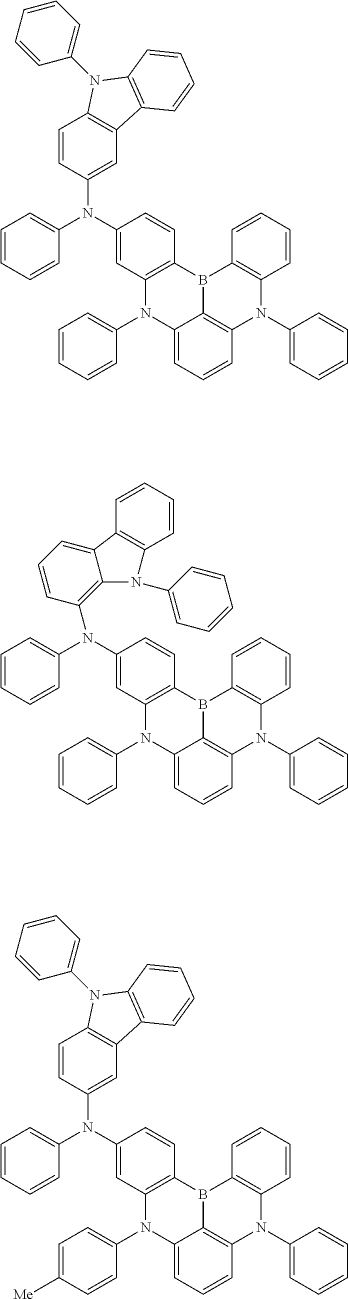 [SMILES: c1ccc(N(c2ccc3c(c2)N(c2ccccc2)c2cccc4c2B3c2ccccc2N4c2ccccc2)c2ccc3c(c2)c2ccccc2n3-c2ccccc2)cc1 ; c1ccc(N2c3ccccc3B3c4ccc(N(c5ccccc5)c5cccc6c7ccccc7n(-c7ccccc7)c56)cc4N(c4ccccc4)c4cccc2c43)cc1 ; Cc1ccc(N2c3cc(N(c4ccccc4)c4ccc5c(c4)c4ccccc4n5-c4ccccc4)ccc3B3c4ccccc4N(c4ccccc4)c4cccc2c43)cc1]

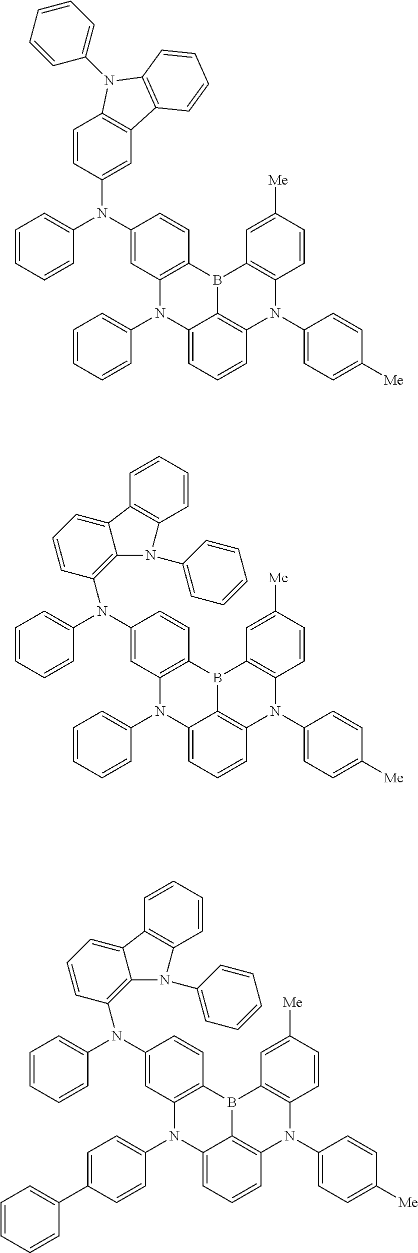 [SMILES: Cc1ccc(N2c3ccc(C)cc3B3c4ccc(N(c5ccccc5)c5ccc6c(c5)c5ccccc5n6-c5ccccc5)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccc(-c5ccccc5)cc4)c4cccc2c43)cc1]

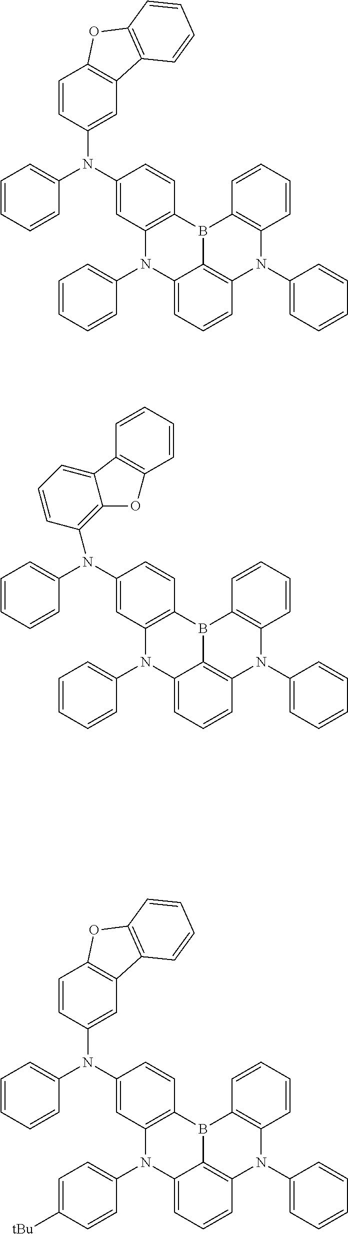 [SMILES: c1ccc(N(c2ccc3c(c2)N(c2ccccc2)c2cccc4c2B3c2ccccc2N4c2ccccc2)c2ccc3oc4ccccc4c3c2)cc1 ; c1ccc(N2c3ccccc3B3c4ccc(N(c5ccccc5)c5cccc6c5oc5ccccc56)cc4N(c4ccccc4)c4cccc2c43)cc1 ; CC(C)(C)c1ccc(N2c3cc(N(c4ccccc4)c4ccc5oc6ccccc6c5c4)ccc3B3c4ccccc4N(c4ccccc4)c4cccc2c43)cc1]

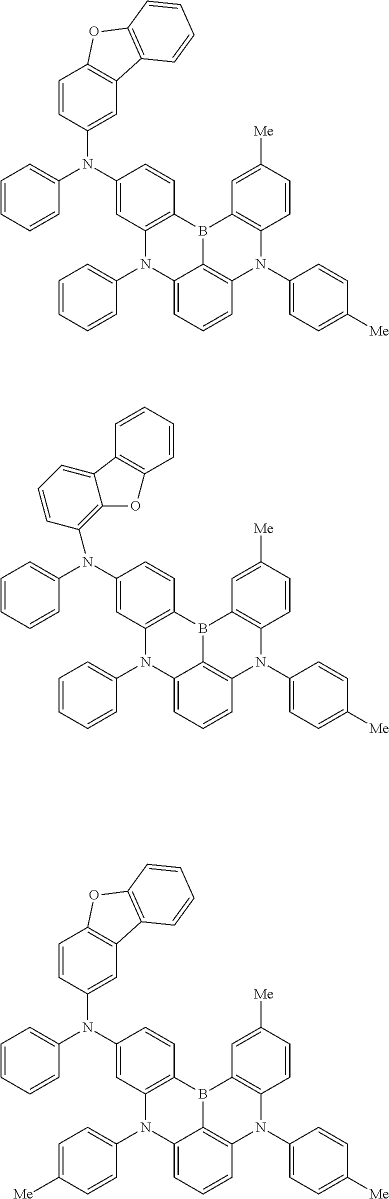 [SMILES: Cc1ccc(N2c3ccc(C)cc3B3c4ccc(N(c5ccccc5)c5ccc6oc7ccccc7c6c5)cc4N(c4ccccc4)c4cccc2c43)cc1 ; Cc1ccc(N2c3ccc(C)cc3B3c4ccc(N(c5ccccc5)c5cccc6c5oc5ccccc56)cc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccc(C)cc4)c4cccc2c43)cc1]

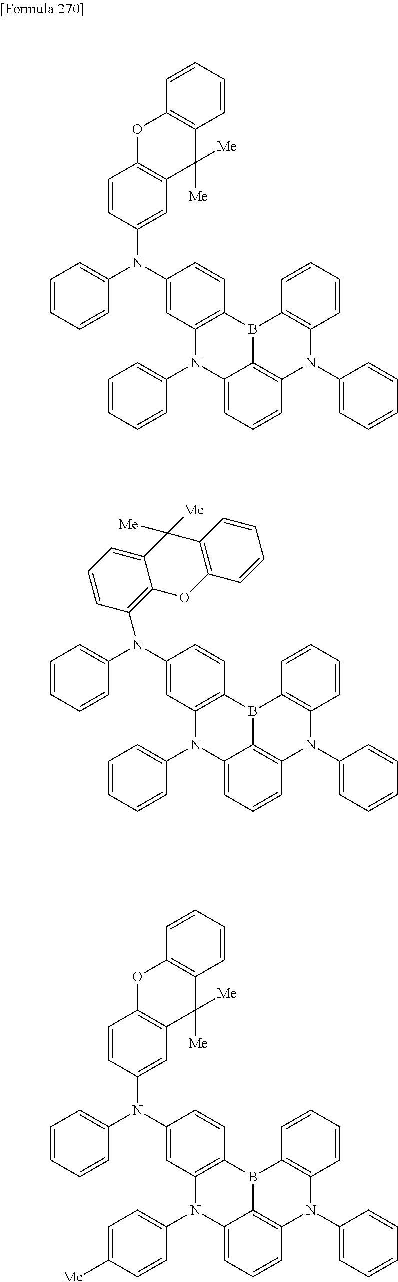 [SMILES: CC1(C)c2ccccc2Oc2ccc(N(c3ccccc3)c3ccc4c(c3)N(c3ccccc3)c3cccc5c3B4c3ccccc3N5c3ccccc3)cc21 ; CC1(C)c2ccccc2Oc2c(N(c3ccccc3)c3ccc4c(c3)N(c3ccccc3)c3cccc5c3B4c3ccccc3N5c3ccccc3)cccc21 ; Cc1ccc(N2c3cc(N(c4ccccc4)c4ccc5c(c4)C(C)(C)c4ccccc4O5)ccc3B3c4ccccc4N(c4ccccc4)c4cccc2c43)cc1]

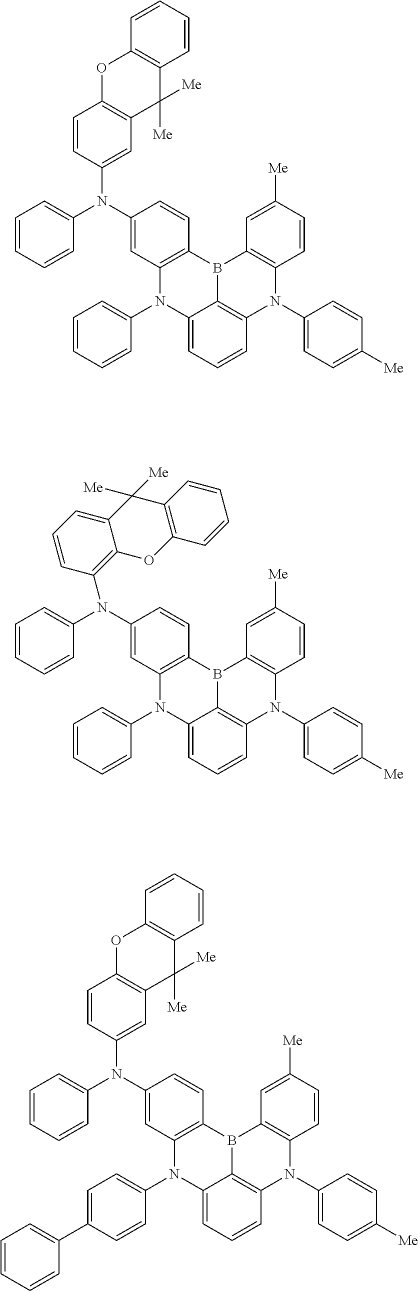 [SMILES: Cc1ccc(N2c3ccc(C)cc3B3c4ccc(N(c5ccccc5)c5ccc6c(c5)C(C)(C)c5ccccc5O6)cc4N(c4ccccc4)c4cccc2c43)cc1 ; Cc1ccc(N2c3ccc(C)cc3B3c4ccc(N(c5ccccc5)c5cccc6c5Oc5ccccc5C6(C)C)cc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccc(-c5ccccc5)cc4)c4cccc2c43)cc1]

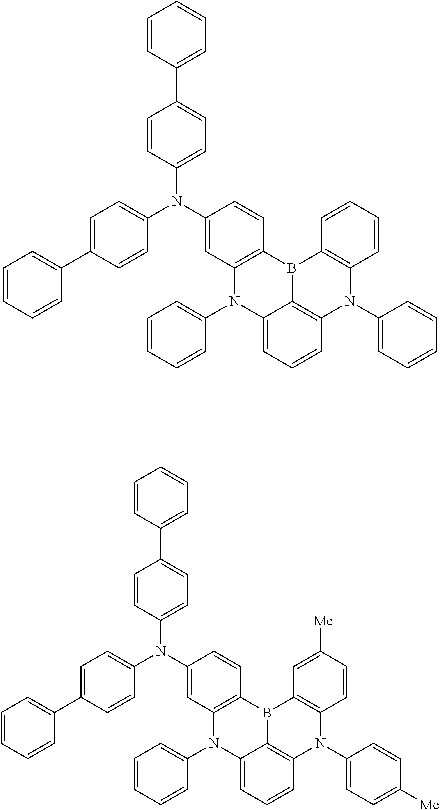 [SMILES: c1ccc(-c2ccc(N(c3ccc(-c4ccccc4)cc3)c3ccc4c(c3)N(c3ccccc3)c3cccc5c3B4c3ccccc3N5c3ccccc3)cc2)cc1 ; Cc1ccc(N2c3ccc(C)cc3B3c4ccc(N(c5ccc(-c6ccccc6)cc5)c5ccc(-c6ccccc6)cc5)cc4N(c4ccccc4)c4cccc2c43)cc1]

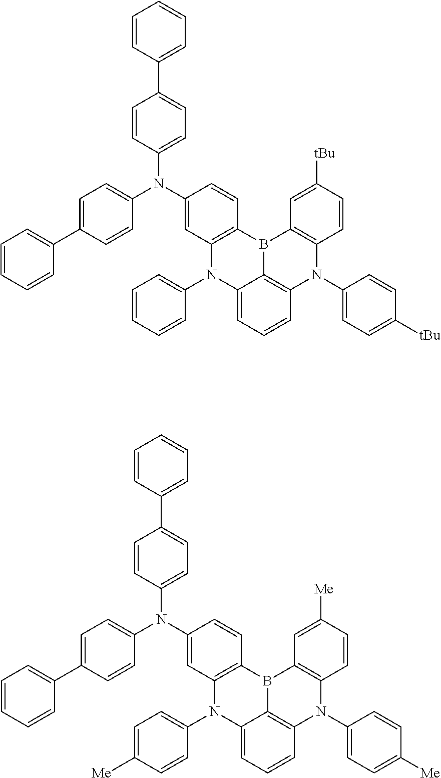 [SMILES: CC(C)(C)c1ccc(N2c3ccc(C(C)(C)C)cc3B3c4ccc(N(c5ccc(-c6ccccc6)cc5)c5ccc(-c6ccccc6)cc5)cc4N(c4ccccc4)c4cccc2c43)cc1 ; Cc1ccc(N2c3ccc(C)cc3B3c4ccc(N(c5ccc(-c6ccccc6)cc5)c5ccc(-c6ccccc6)cc5)cc4N(c4ccc(C)cc4)c4cccc2c43)cc1]

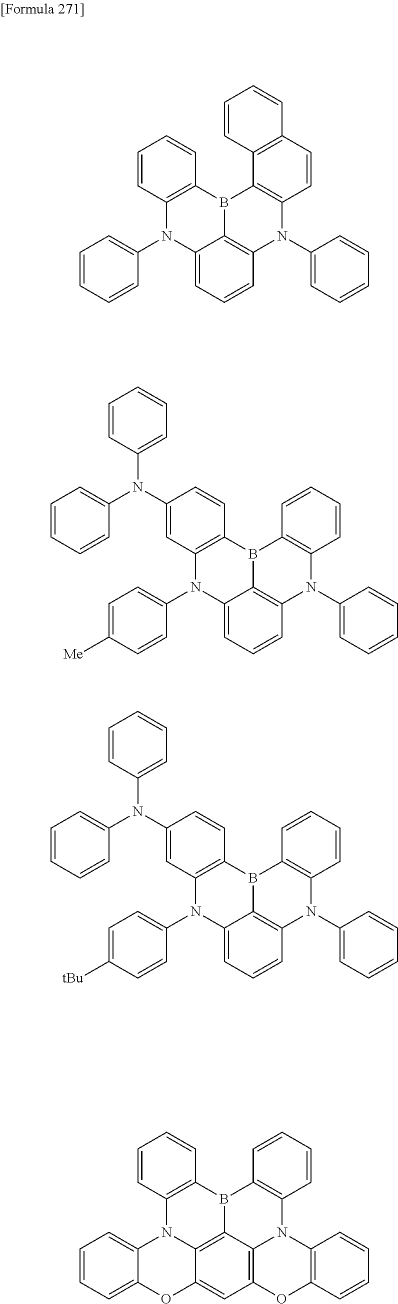 [SMILES: c1ccc(N2c3ccccc3B3c4c2cccc4N(c2ccccc2)c2ccc4ccccc4c23)cc1 ; Cc1ccc(N2c3cc(N(c4ccccc4)c4ccccc4)ccc3B3c4ccccc4N(c4ccccc4)c4cccc2c43)cc1 ; CC(C)(C)c1ccc(N2c3cc(N(c4ccccc4)c4ccccc4)ccc3B3c4ccccc4N(c4ccccc4)c4cccc2c43)cc1 ; c1ccc2c(c1)B1c3ccccc3-n3c4ccccc4oc4cc5oc6ccccc6n-2c5c1c43]

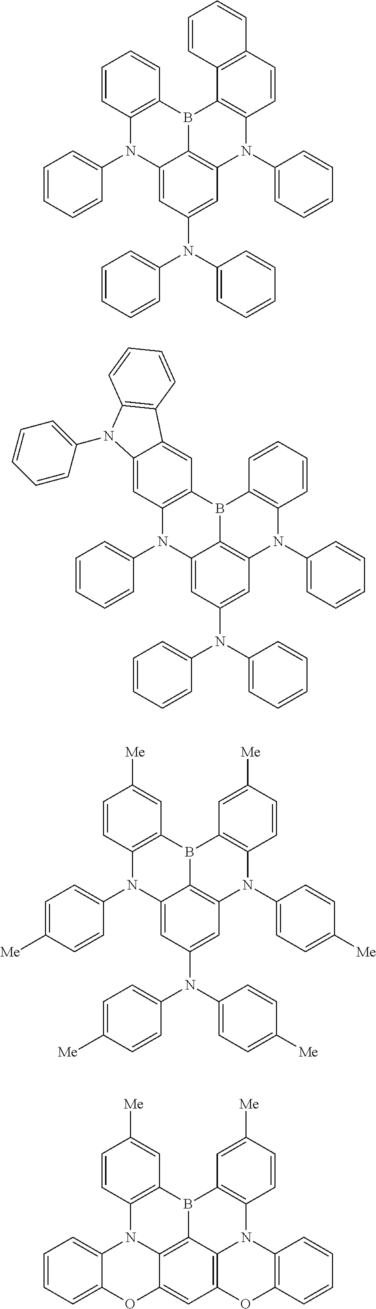

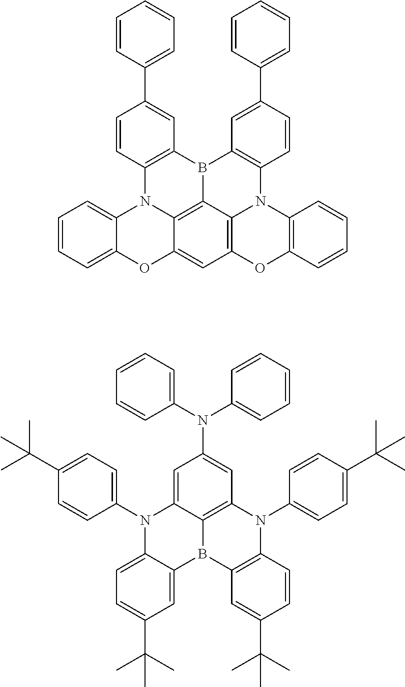 [SMILES: c1ccc(-c2ccc3c(c2)B2c4cc(-c5ccccc5)ccc4-n4c5ccccc5oc5cc6oc7ccccc7n-3c6c2c54)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1]

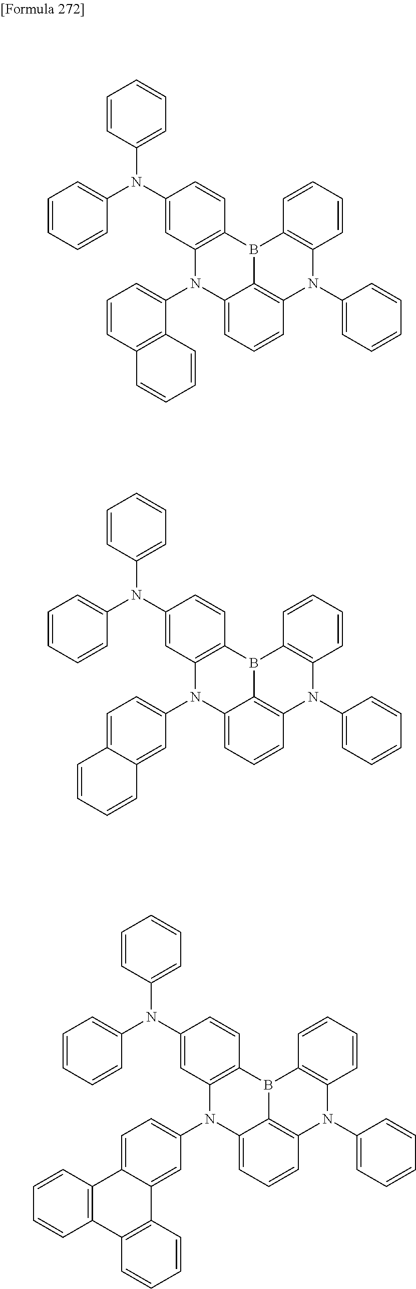 [SMILES: c1ccc(N(c2ccccc2)c2ccc3c(c2)N(c2cccc4ccccc24)c2cccc4c2B3c2ccccc2N4c2ccccc2)cc1 ; c1ccc(N(c2ccccc2)c2ccc3c(c2)N(c2ccc4ccccc4c2)c2cccc4c2B3c2ccccc2N4c2ccccc2)cc1 ; c1ccc(N(c2ccccc2)c2ccc3c(c2)N(c2ccc4c5ccccc5c5ccccc5c4c2)c2cccc4c2B3c2ccccc2N4c2ccccc2)cc1]

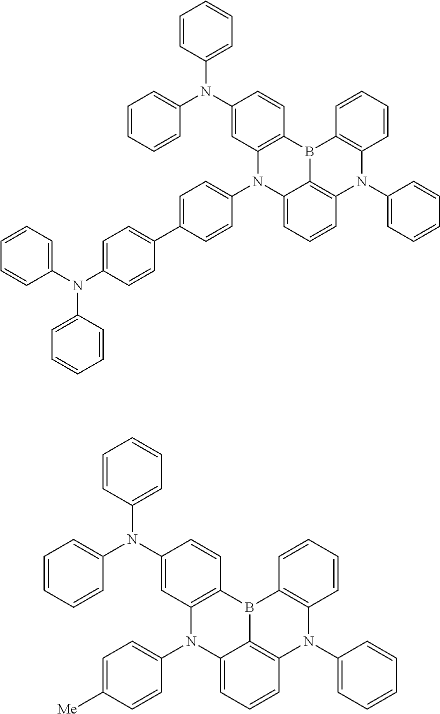 [SMILES: c1ccc(N(c2ccccc2)c2ccc(-c3ccc(N4c5cc(N(c6ccccc6)c6ccccc6)ccc5B5c6ccccc6N(c6ccccc6)c6cccc4c65)cc3)cc2)cc1 ; Cc1ccc(N2c3cc(N(c4ccccc4)c4ccccc4)ccc3B3c4ccccc4N(c4ccccc4)c4cccc2c43)cc1]

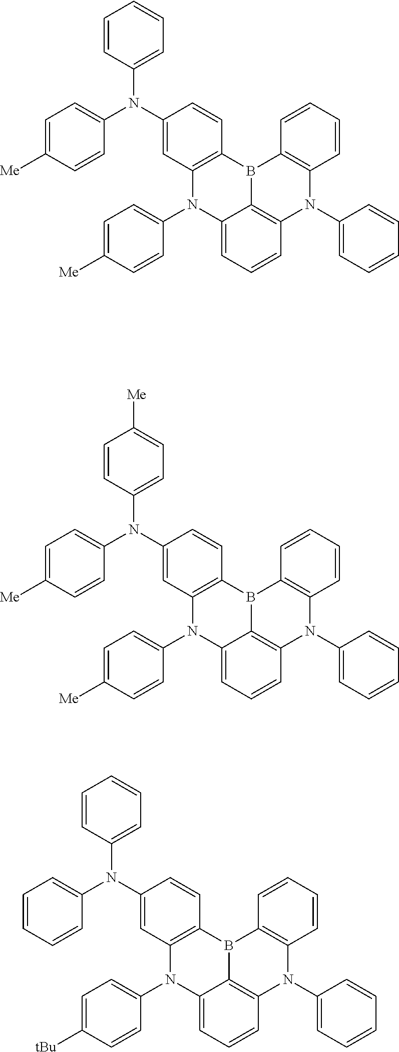 [SMILES: Cc1ccc(N(c2ccccc2)c2ccc3c(c2)N(c2ccc(C)cc2)c2cccc4c2B3c2ccccc2N4c2ccccc2)cc1 ; Cc1ccc(N(c2ccc(C)cc2)c2ccc3c(c2)N(c2ccc(C)cc2)c2cccc4c2B3c2ccccc2N4c2ccccc2)cc1 ; CC(C)(C)c1ccc(N2c3cc(N(c4ccccc4)c4ccccc4)ccc3B3c4ccccc4N(c4ccccc4)c4cccc2c43)cc1]

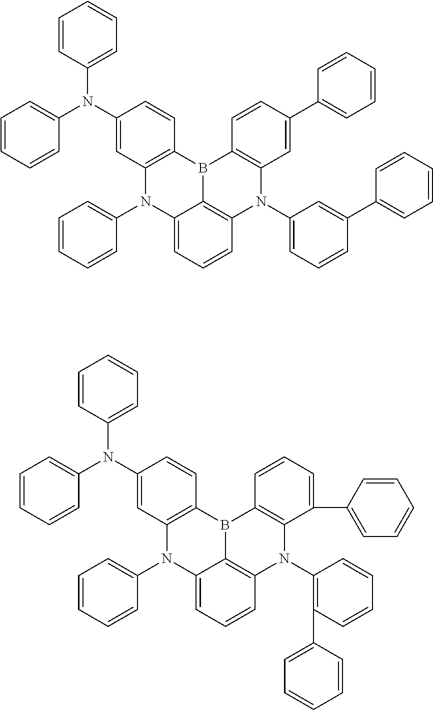 [SMILES: c1ccc(-c2cccc(N3c4cc(-c5ccccc5)ccc4B4c5ccc(N(c6ccccc6)c6ccccc6)cc5N(c5ccccc5)c5cccc3c54)c2)cc1 ; c1ccc(-c2ccccc2N2c3cccc4c3B(c3ccc(N(c5ccccc5)c5ccccc5)cc3N4c3ccccc3)c3cccc(-c4ccccc4)c32)cc1]

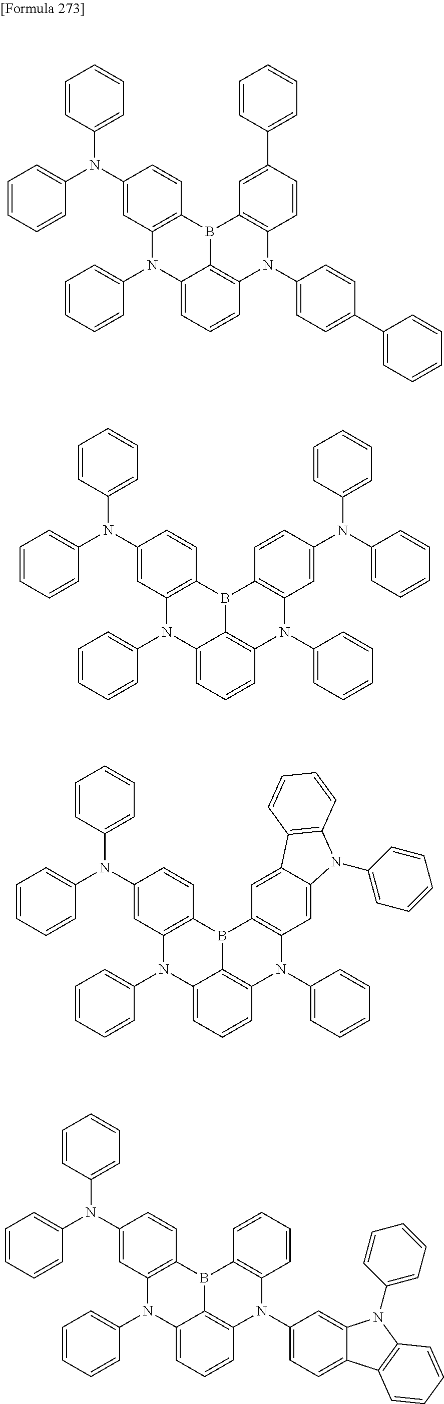 [SMILES: c1ccc(-c2ccc(N3c4ccc(-c5ccccc5)cc4B4c5ccc(N(c6ccccc6)c6ccccc6)cc5N(c5ccccc5)c5cccc3c54)cc2)cc1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1 ; c1ccc(N(c2ccccc2)c2ccc3c(c2)N(c2ccccc2)c2cccc4c2B3c2cc3c5ccccc5n(-c5ccccc5)c3cc2N4c2ccccc2)cc1 ; c1ccc(N(c2ccccc2)c2ccc3c(c2)N(c2ccccc2)c2cccc4c2B3c2ccccc2N4c2ccc3c4ccccc4n(-c4ccccc4)c3c2)cc1]

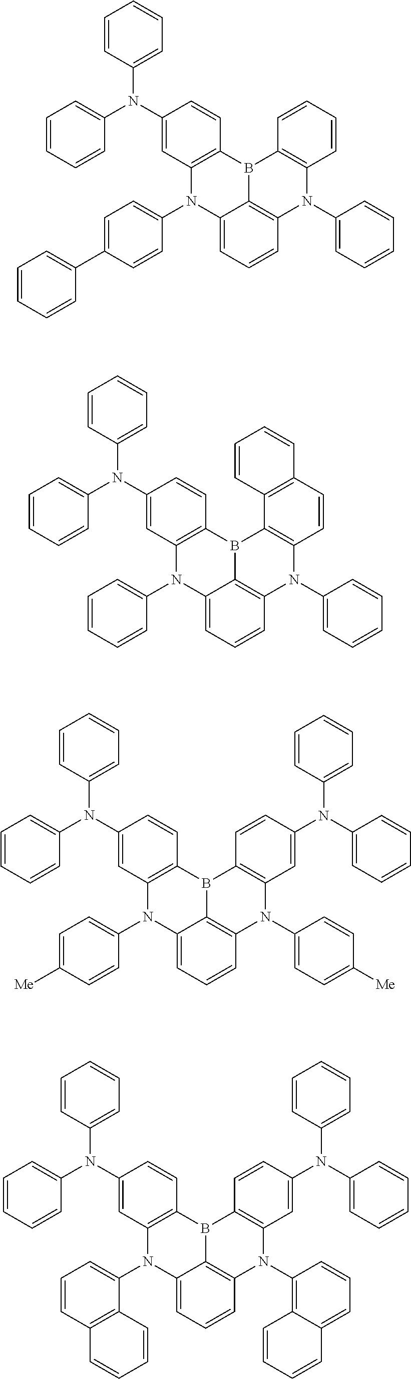 [SMILES: c1ccc(-c2ccc(N3c4cc(N(c5ccccc5)c5ccccc5)ccc4B4c5ccccc5N(c5ccccc5)c5cccc3c54)cc2)cc1 ; c1ccc(N(c2ccccc2)c2ccc3c(c2)N(c2ccccc2)c2cccc4c2B3c2c(ccc3ccccc23)N4c2ccccc2)cc1 ; Cc1ccc(N2c3cc(N(c4ccccc4)c4ccccc4)ccc3B3c4ccc(N(c5ccccc5)c5ccccc5)cc4N(c4ccc(C)cc4)c4cccc2c43)cc1 ; c1ccc(N(c2ccccc2)c2ccc3c(c2)N(c2cccc4ccccc24)c2cccc4c2B3c2ccc(N(c3ccccc3)c3ccccc3)cc2N4c2cccc3ccccc23)cc1]

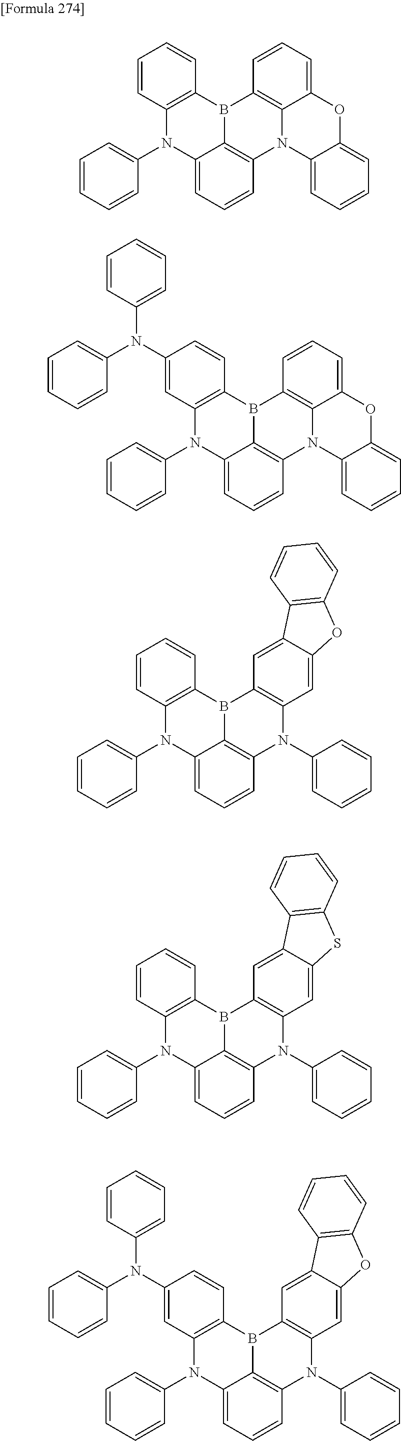

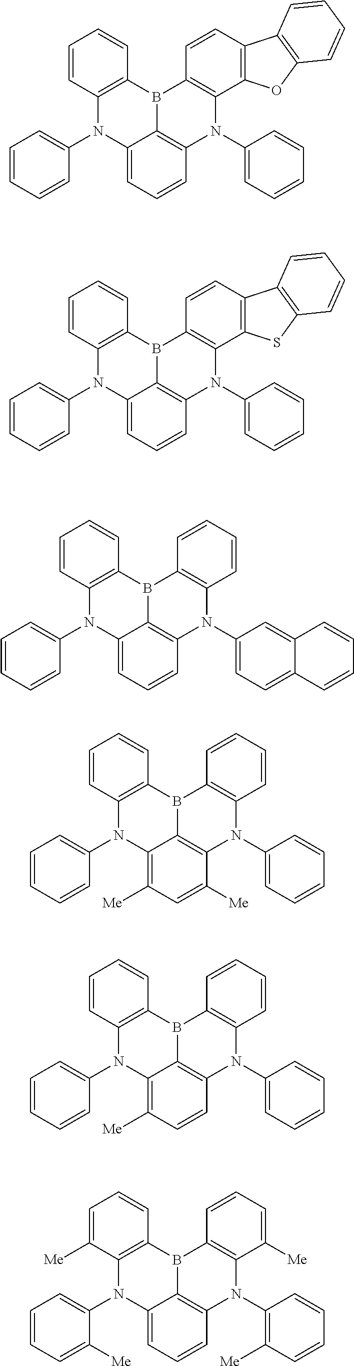 [SMILES: c1ccc(N2c3ccccc3B3c4ccc5c(oc6ccccc65)c4N(c4ccccc4)c4cccc2c43)cc1 ; c1ccc(N2c3ccccc3B3c4ccc5c(sc6ccccc65)c4N(c4ccccc4)c4cccc2c43)cc1 ; c1ccc(N2c3ccccc3B3c4ccccc4N(c4ccc5ccccc5c4)c4cccc2c43)cc1 ; Cc1cc(C)c2c3c1N(c1ccccc1)c1ccccc1B3c1ccccc1N2c1ccccc1 ; Cc1ccc2c3c1N(c1ccccc1)c1ccccc1B3c1ccccc1N2c1ccccc1 ; Cc1ccccc1N1c2cccc3c2B(c2cccc(C)c21)c1cccc(C)c1N3c1ccccc1C]

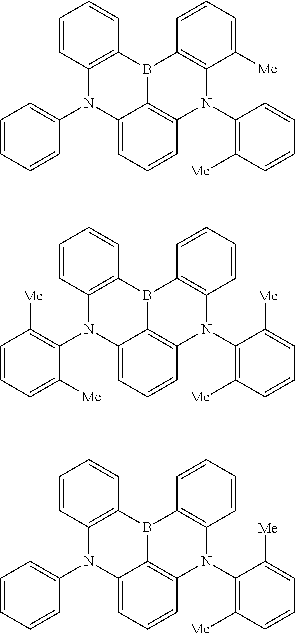 [SMILES: Cc1ccccc1N1c2cccc3c2B(c2ccccc2N3c2ccccc2)c2cccc(C)c21 ; Cc1cccc(C)c1N1c2ccccc2B2c3ccccc3N(c3c(C)cccc3C)c3cccc1c32 ; Cc1cccc(C)c1N1c2ccccc2B2c3ccccc3N(c3ccccc3)c3cccc1c32]

### Compound Represented by Formula (7)

The compound represented by the formula (7) will be described below.

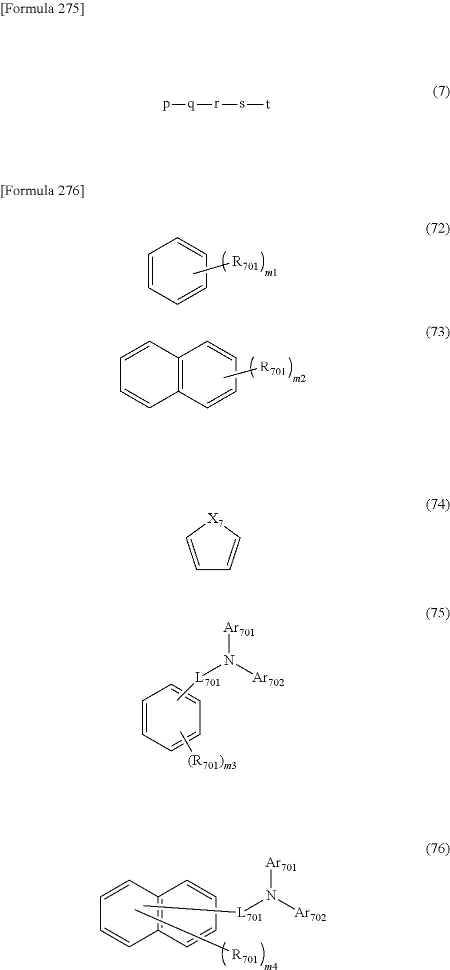

In the formula (7): r ring is a ring represented by the formula (72) or the formula (73), the r ring being fused with at any position(s) of respective adjacent rings;
q ring and s ring are each independently a ring represented by the formula (74) and fused with any position(s) of respective adjacent rings;
p ring and t ring are each independently a moiety represented by the formula (75) or the formula (76) and fused with any position(s) of respective adjacent rings;
X₇ is an oxygen atom, a sulfur atom, or NR₇₀₂;
when a plurality of R₇₀₁ are present, adjacent ones of the plurality of R₇₀₁ are mutually bonded to form a substituted or unsubstituted monocyclic ring, mutually bonded to form a substituted or unsubstituted fused ring, or not mutually bonded;
R₇₀₁ and R₇₀₂ not forming the monocyclic ring and not forming the fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
Ar₇₀₁ and Ar₇₀₂ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
L₇₀₁ is a substituted or unsubstituted alkylene group having 1 to 50 carbon atoms, a substituted or unsubstituted alkenylene group having 2 to 50 carbon atoms, a substituted or unsubstituted alkynylene group having 2 to 50 carbon atoms, a substituted or unsubstituted cycloalkylene group having 3 to 50 ring carbon atoms, a substituted or unsubstituted arylene group having 6 to 50 ring carbon atoms, or a substituted or unsubstituted divalent heterocyclic group having 5 to 50 ring atoms;
m1 is 0, 1, or 2;
m2 is 0, 1, 2, 3, or 4;
m3 is each independently 0, 1, 2, 3 or 3;
m4 is each independently 0, 1, 2, 3, 4, or 5;
when a plurality of R₇₀₁ are present, the plurality of R₇₀₁ are mutually the same or different;
when a plurality of X₇ are present, the plurality of X₇ are mutually the same or different;
when a plurality of R₇₀₂ are present, the plurality of R₇₀₂ are mutually the same or different;
when a plurality of Ar₇₀₁ are present, the plurality of Ar₇₀₁ are mutually the same or different;
when a plurality of Ar₇₀₂ are present, the plurality of Ar₇₀₂ are mutually the same or different; and
when a plurality of L₇₀₁ are present, the plurality of L₇₀₁ are mutually the same or different.

In the formula (7), each of the p ring, q ring, r ring, s ring, and t ring is fused with an adjacent ring(s) sharing two carbon atoms. The fused position and orientation are not limited but may be defined as required.

In an exemplary embodiment, in the formula (72) or the formula (73) representing the r ring, m1 is 0 or m2 is 0.

In an exemplary embodiment, the compound represented by the formula (7) is represented by any one of formulae (71-1) to (71-6) below.

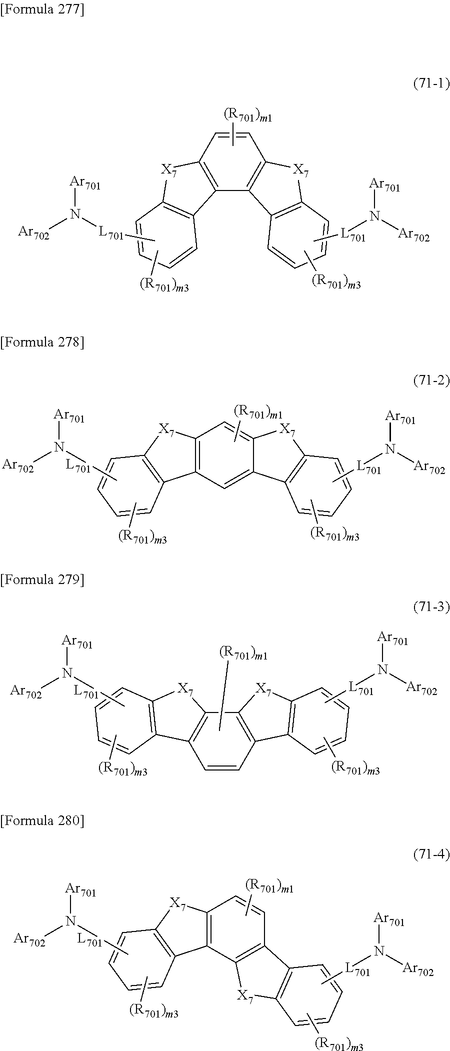

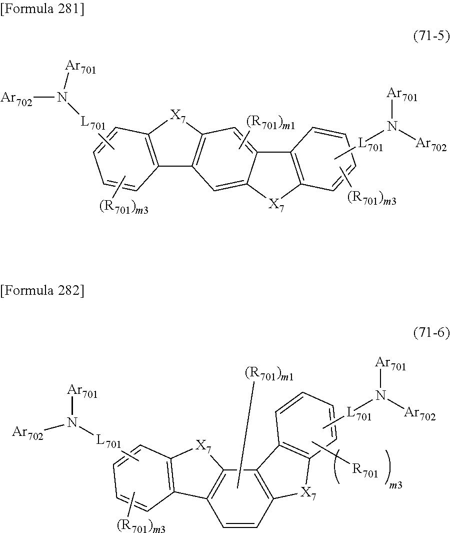

In the formulae (71-1) to (71-6), R₇₀₁, X₇, Ar₇₀₁, Ar₇₀₂, L₇₀₁, m1, and m3 respectively represent the same as R₇₀₁, X₇, Ar₇₀₁, Ar₇₀₂, L₇₀₁, m1, and m3 in the formula (7).

In an exemplary embodiment, the compound represented by the formula (7) is represented by any one of formulae (71-11) to (71-13) below.

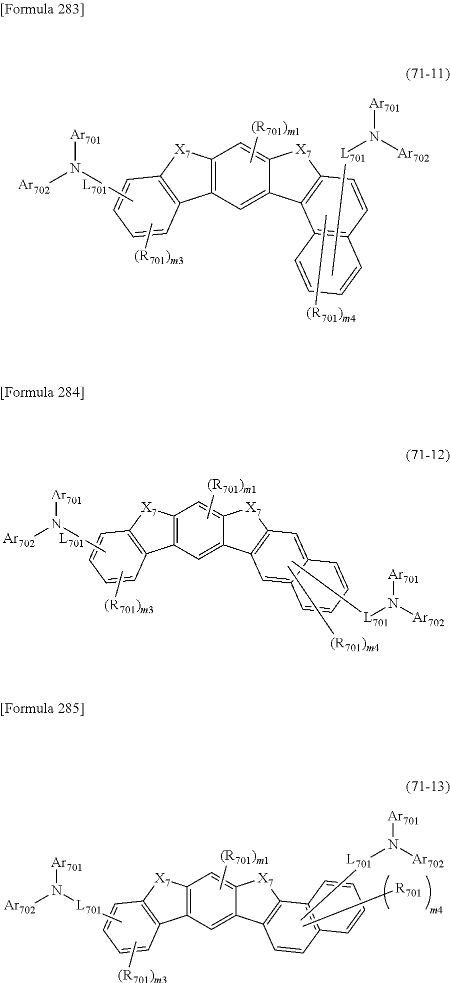

In the formulae (71-11) to (71-13), R₇₀₁, X₇, Ar₇₀₁, Ar₇₀₂, L₇₀₁, m1, m3 and m4 respectively represent the same as R₇₀₁, X₇, Ar₇₀₁, Ar₇₀₂, L₇₀₁, m1, m3 and m4 in the formula (7).

In an exemplary embodiment, the compound represented by the formula (7) is represented by any one of formulae (71-21) to (71-25) below.

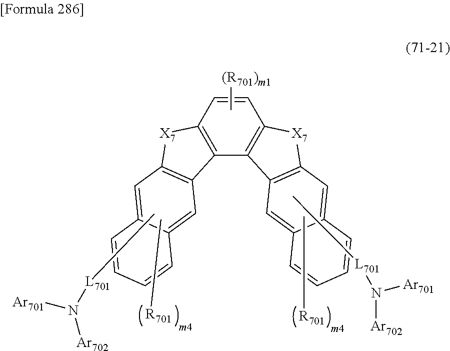

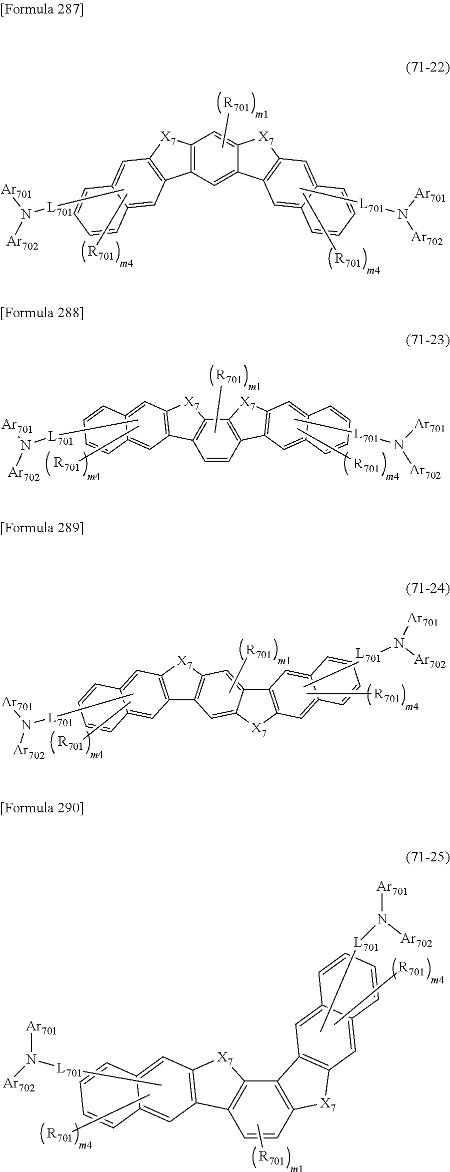

In the formulae (71-21) to (71-25), R₇₀₁, X₇, Ar₇₀₁, Ar₇₀₂, L₇₀₁, m1, and m4 respectively represent the same as R₇₀₁, X₇, Ar₇₀₁, Ar₇₀₂, L₇₀₁, m1, and m4 in the formula (7).

In an exemplary embodiment, the compound represented by the formula (7) is represented by any one of formulae (71-31) to (71-33) below.

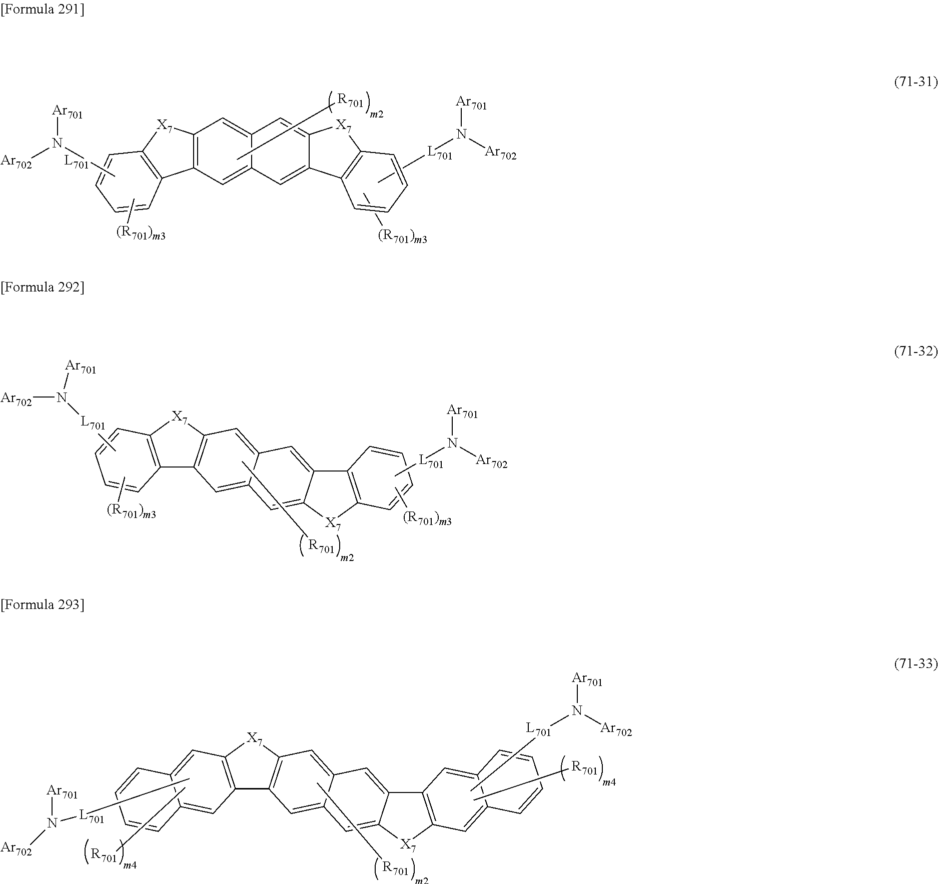

In the formulae (71-31) to (71-33), R₇₀₁, X₇, Ar₇₀₁, Ar₇₀₂, L₇₀₁, and m2 to m4 respectively represent the same as R₇₀₁, X₇, Ar₇₀₁, Ar₇₀₂, L₇₀₁, and m2 to m4 in the formula (7).

In an exemplary embodiment, Ar₇₀₁ and Ar₇₀₂ are each independently a substituted or unsubstituted aryl group having 6 to 50 ring carbon atoms.

In an exemplary embodiment, one of Ar₇₀₁ and Ar₇₀₂ is a substituted or unsubstituted aryl group having 6 to 50 ring carbon atoms, and the other of Ar₇₀₁ and Ar₇₀₂ is a substituted or unsubstituted heterocyclic group having 5 to 50 ring atoms.

### Specific Examples of Compound Represented by Formula (7)

Specific examples of the compound represented by the formula (7) include compounds shown below.

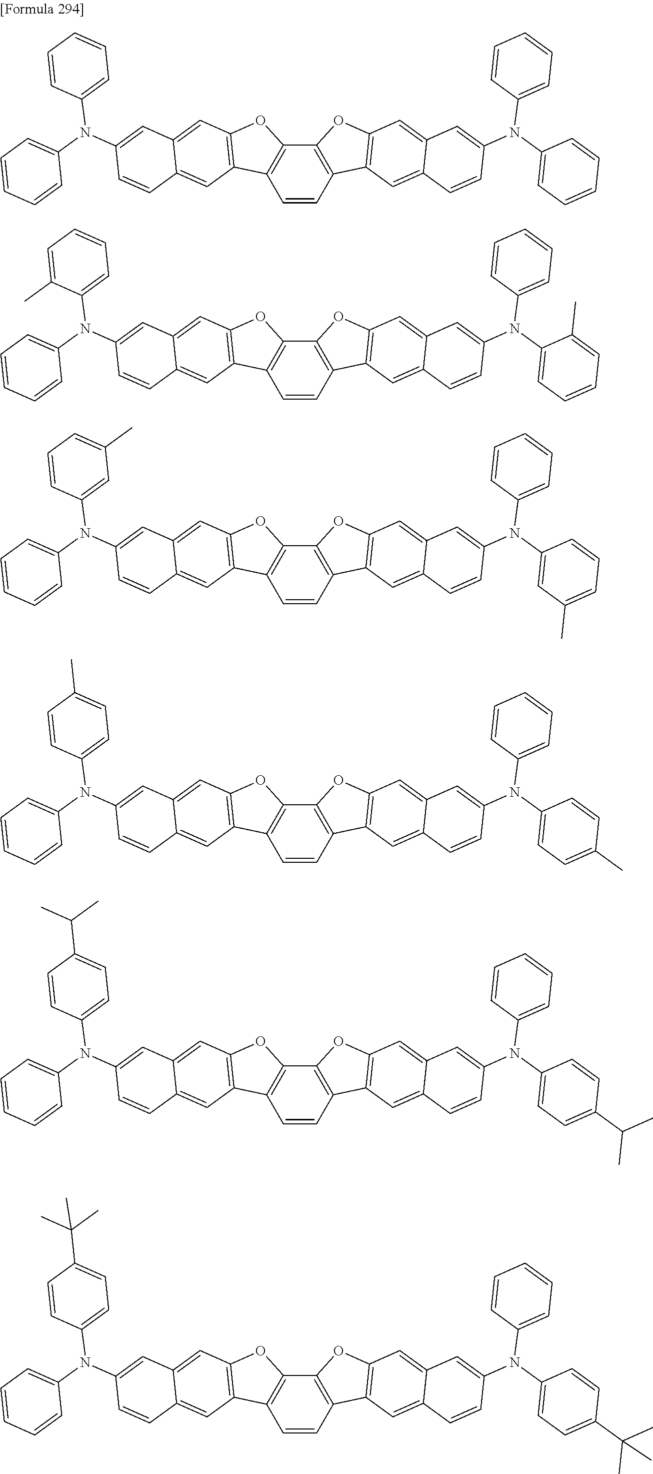

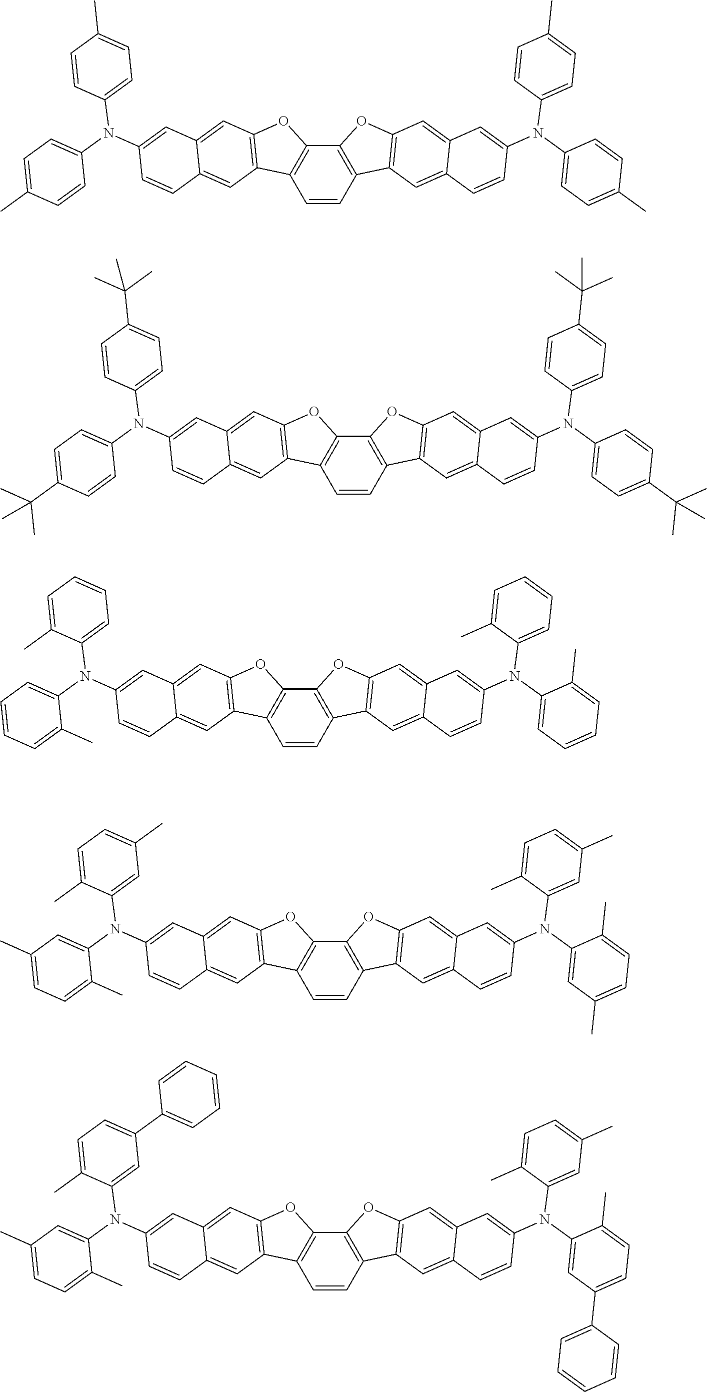

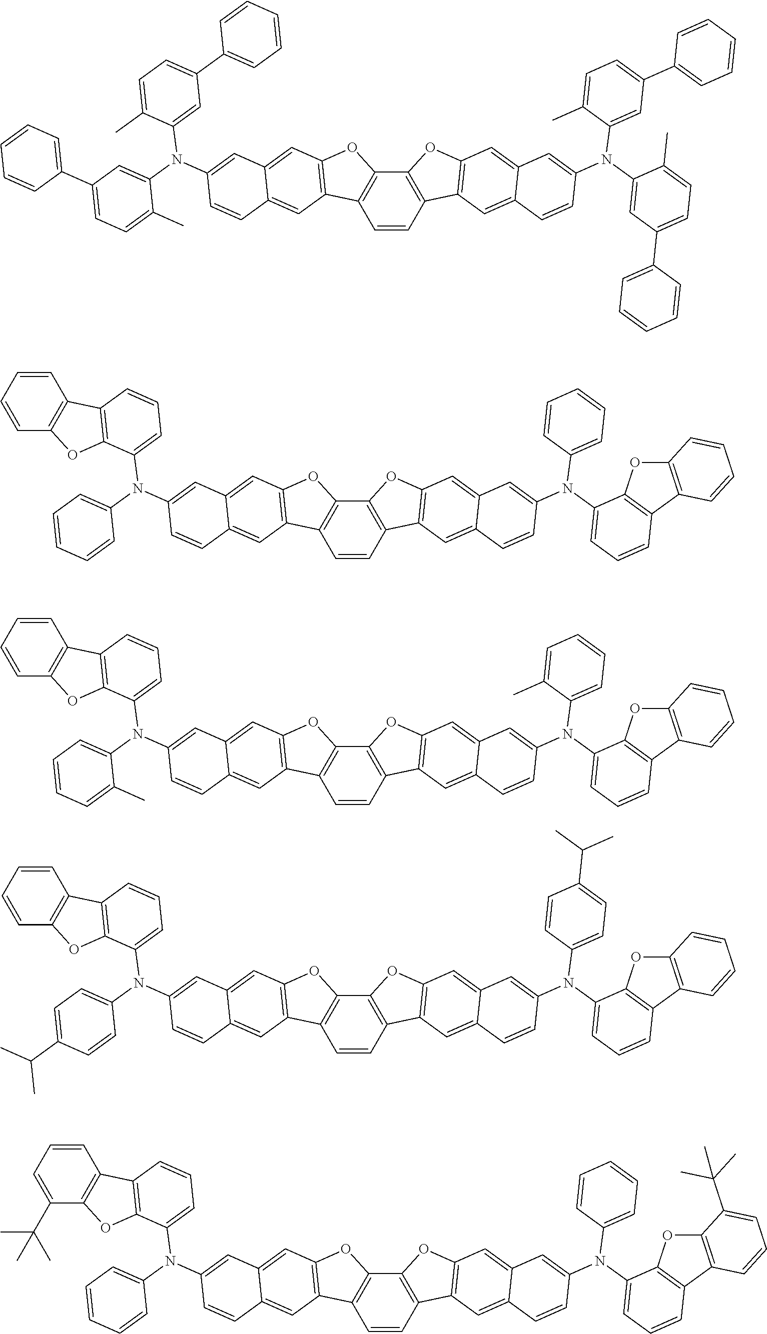

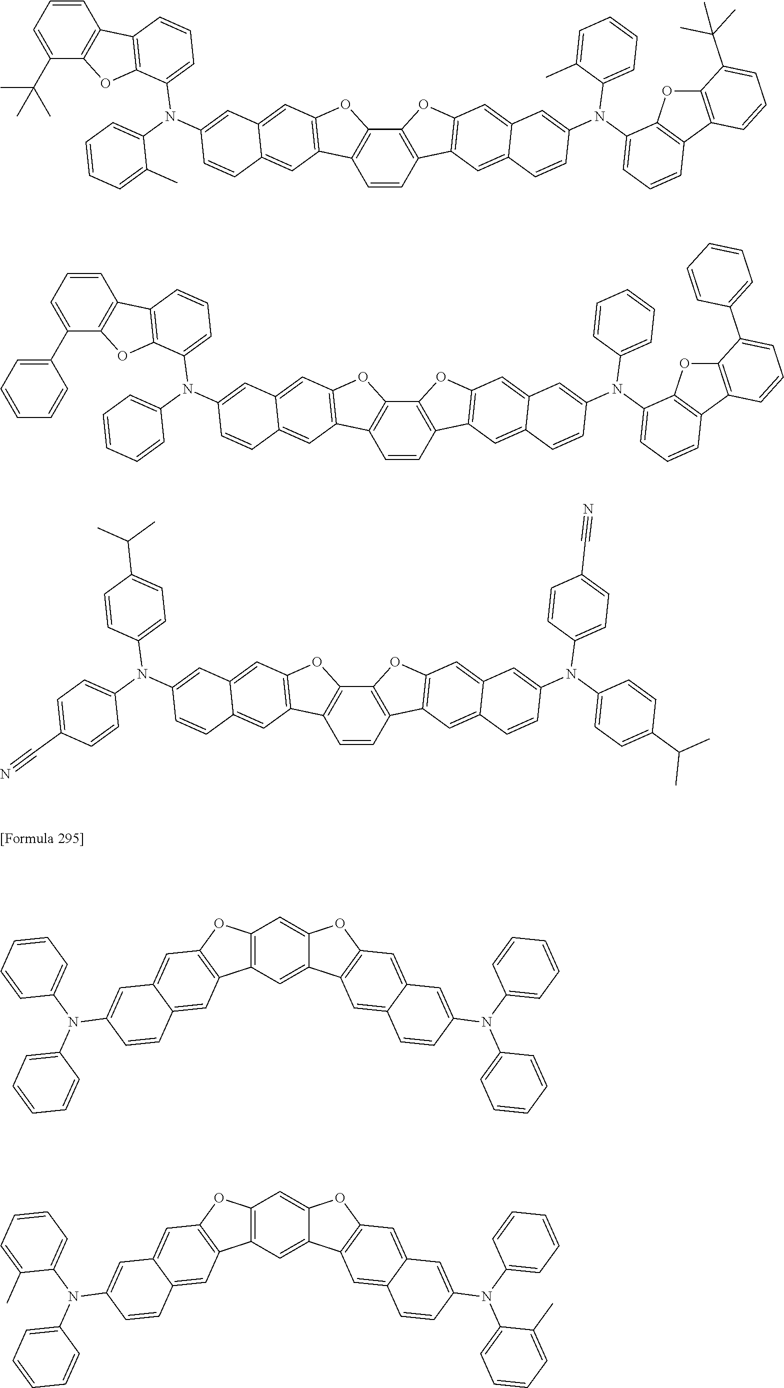

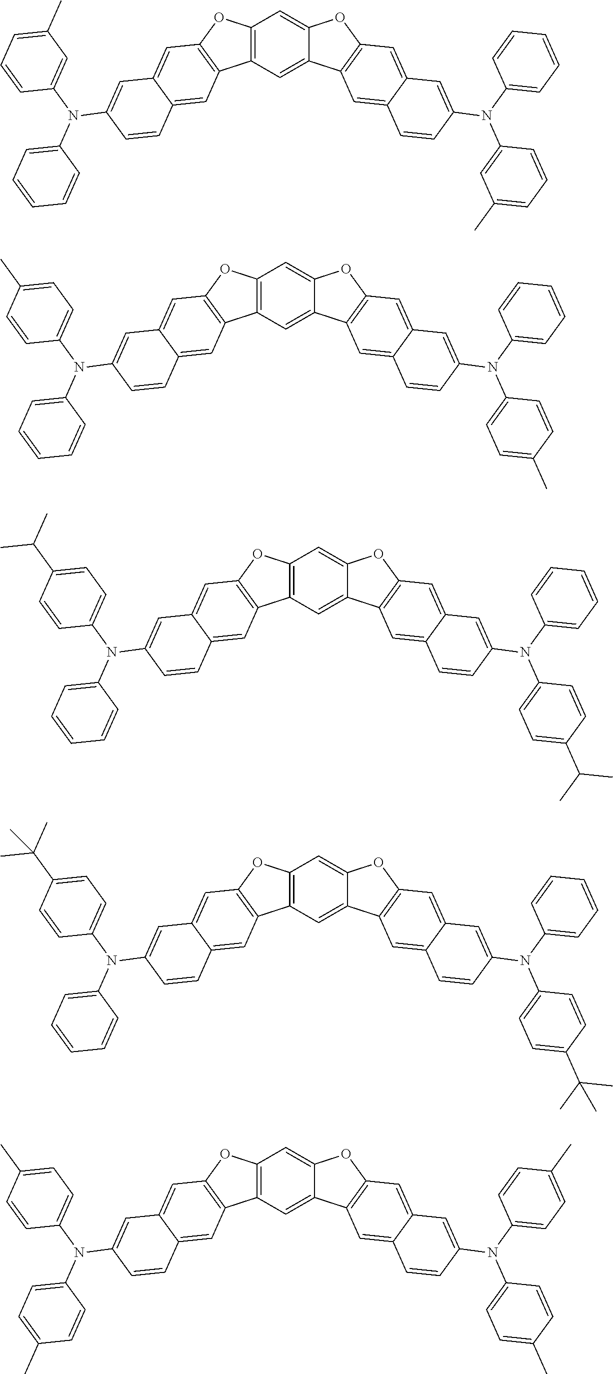

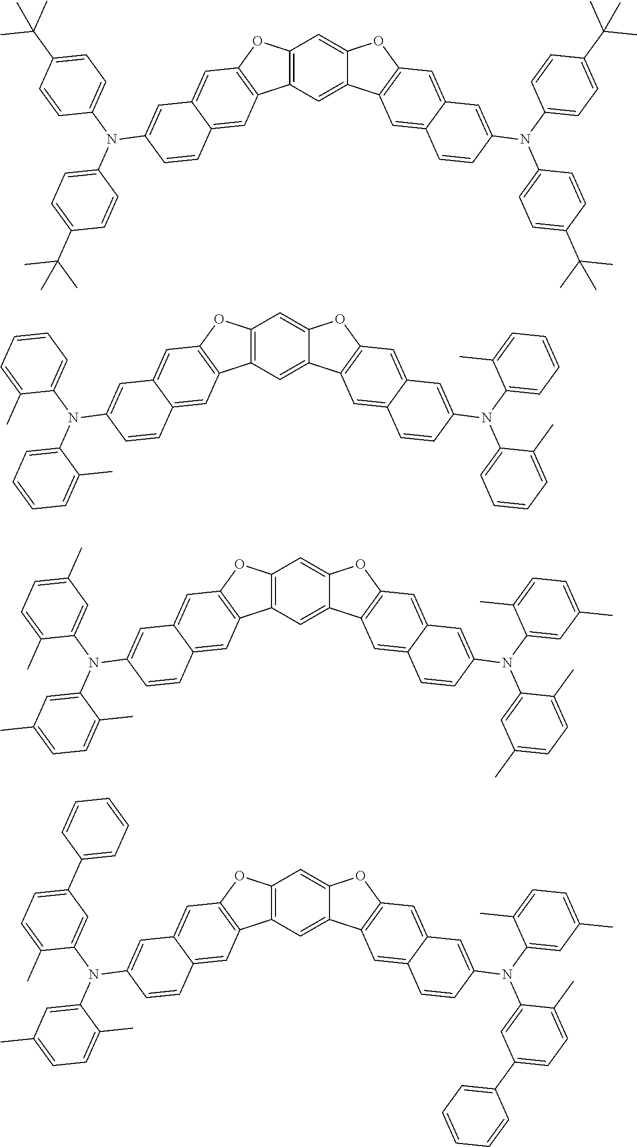

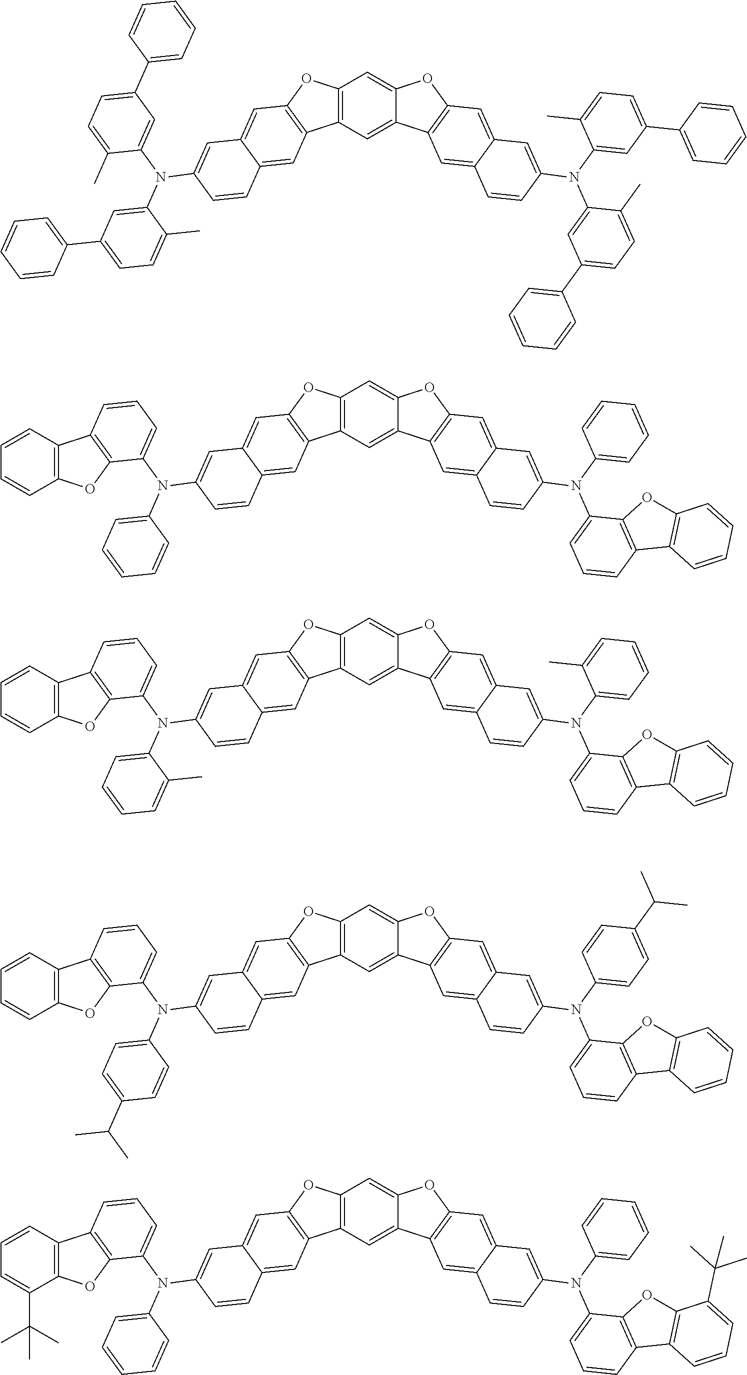

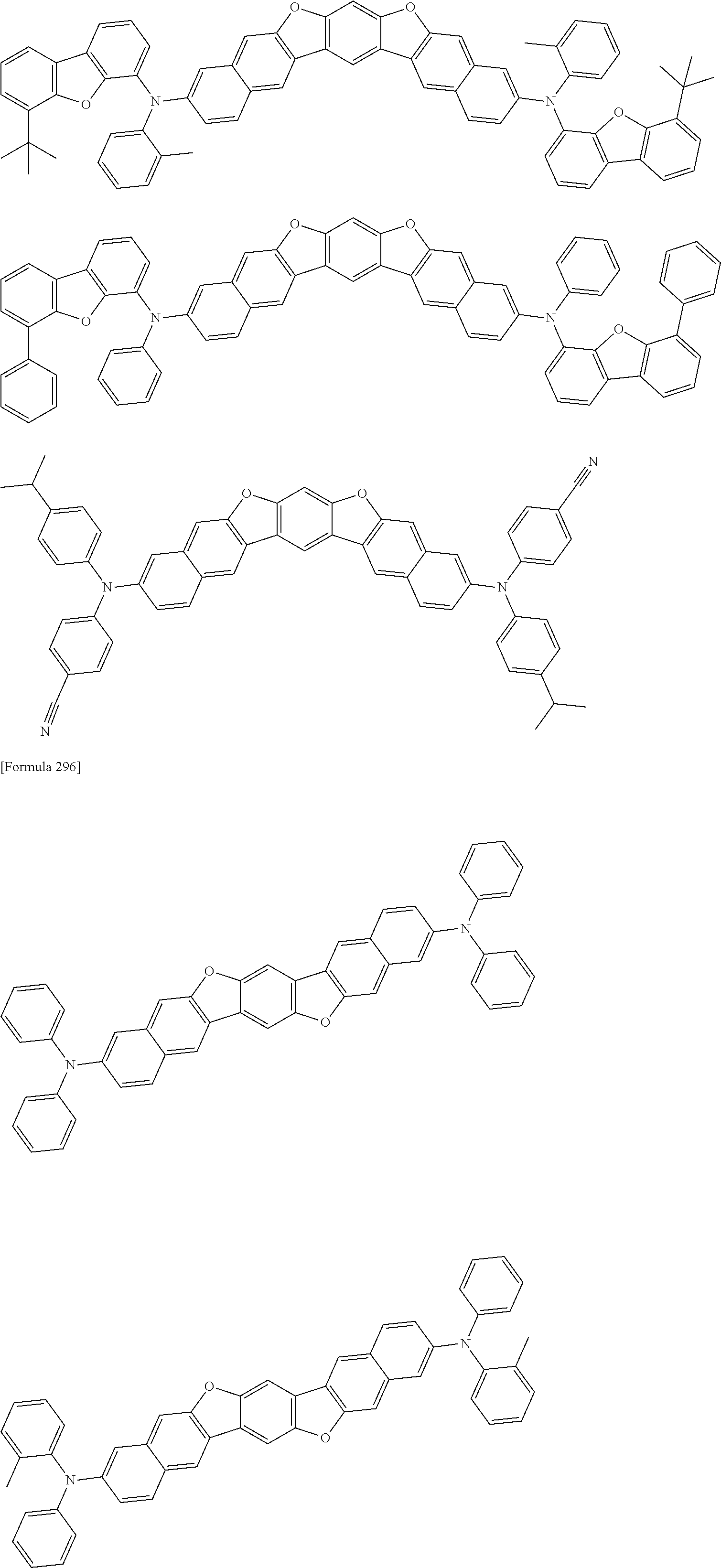

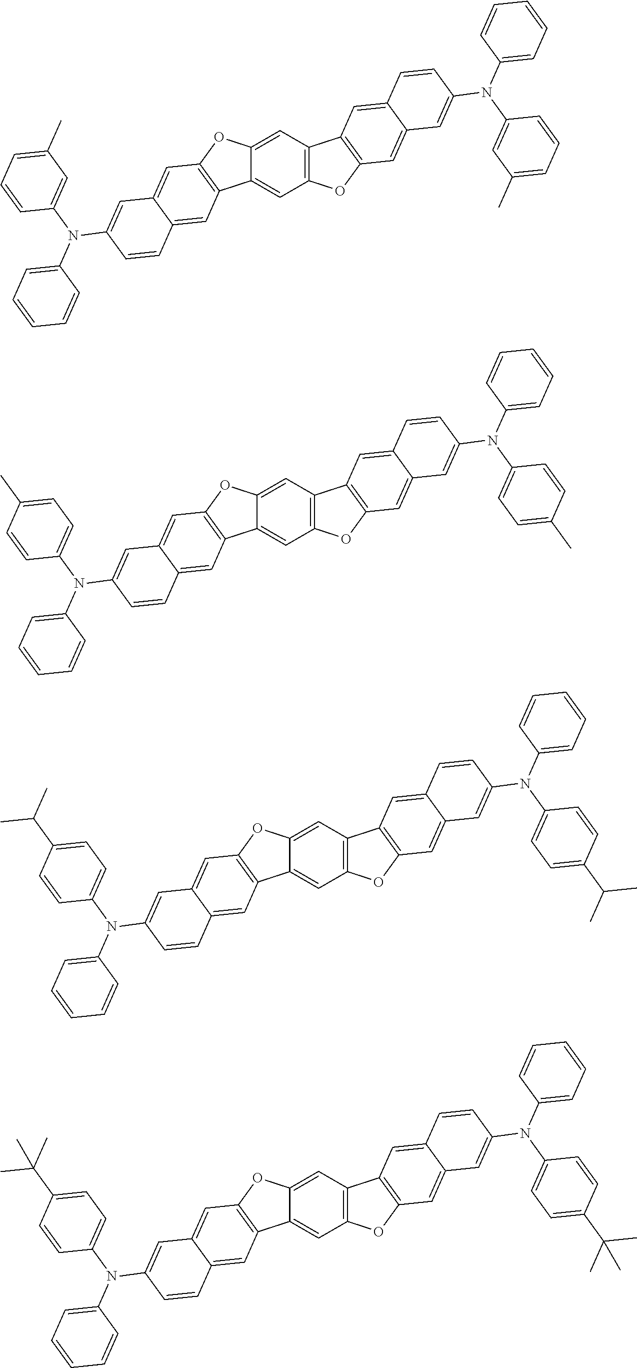

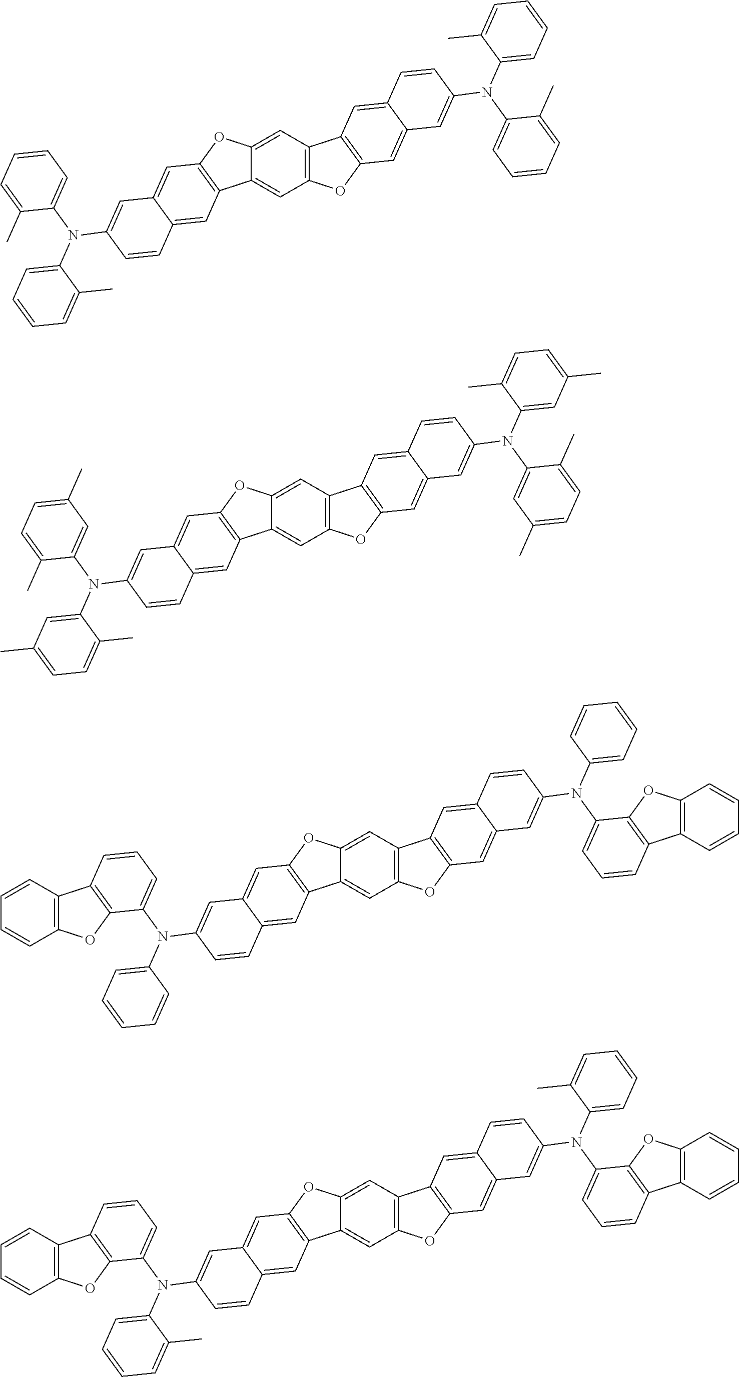

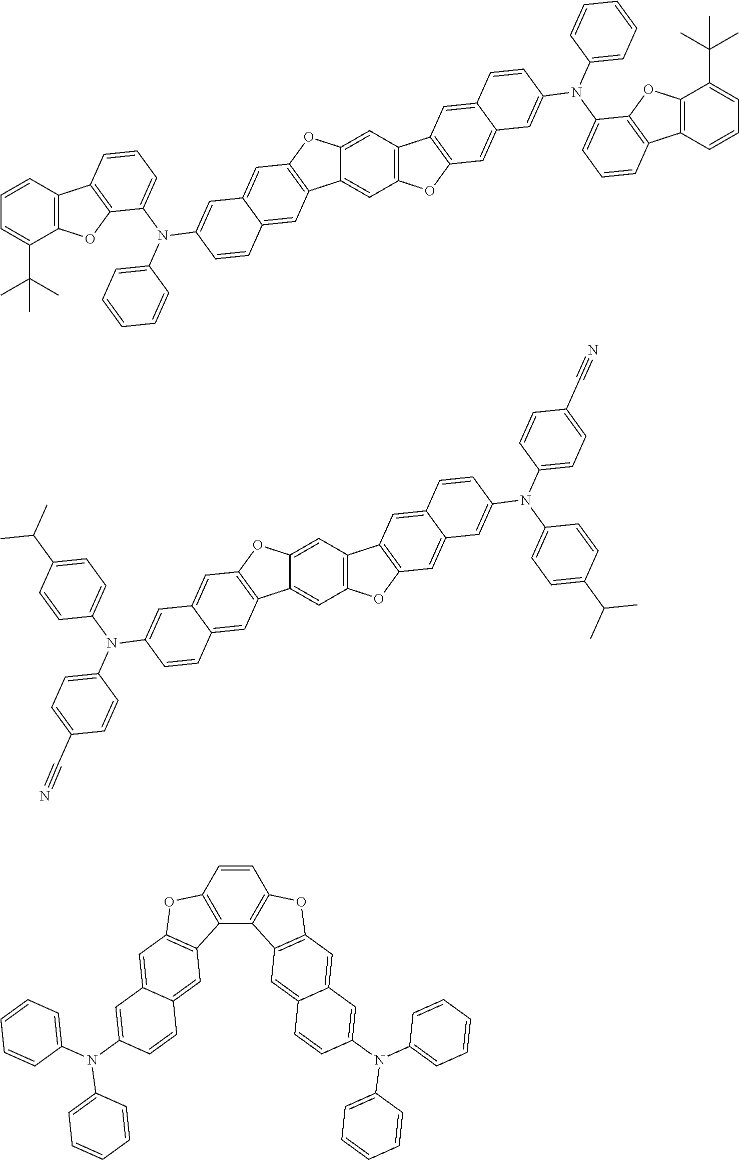

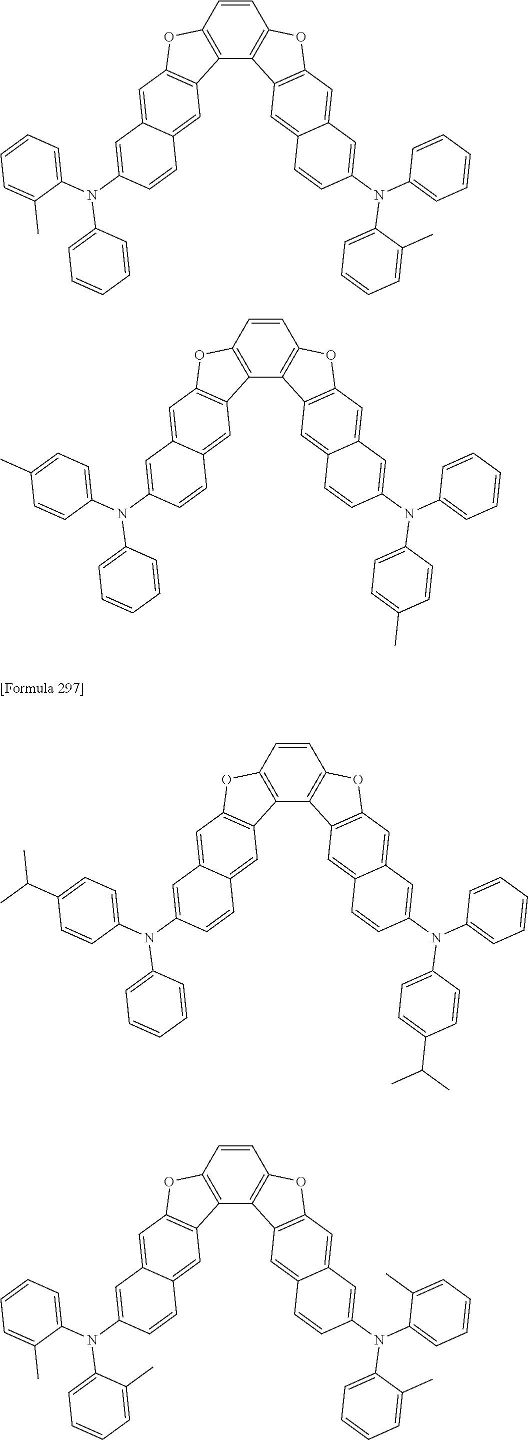 [SMILES: Cc1ccccc1N(c1ccccc1)c1ccc2cc3c(cc2c1)oc1ccc2oc4cc5cc(N(c6ccccc6)c6ccccc6C)ccc5cc4c2c13 ; Cc1ccc(N(c2ccccc2)c2ccc3cc4c(cc3c2)oc2ccc3oc5cc6cc(N(c7ccccc7)c7ccc(C)cc7)ccc6cc5c3c24)cc1 ; CC(C)c1ccc(N(c2ccccc2)c2ccc3cc4c(cc3c2)oc2ccc3oc5cc6cc(N(c7ccccc7)c7ccc(C(C)C)cc7)ccc6cc5c3c24)cc1 ; Cc1ccccc1N(c1ccc2cc3c(cc2c1)oc1ccc2oc4cc5cc(N(c6ccccc6C)c6ccccc6C)ccc5cc4c2c13)c1ccccc1C]

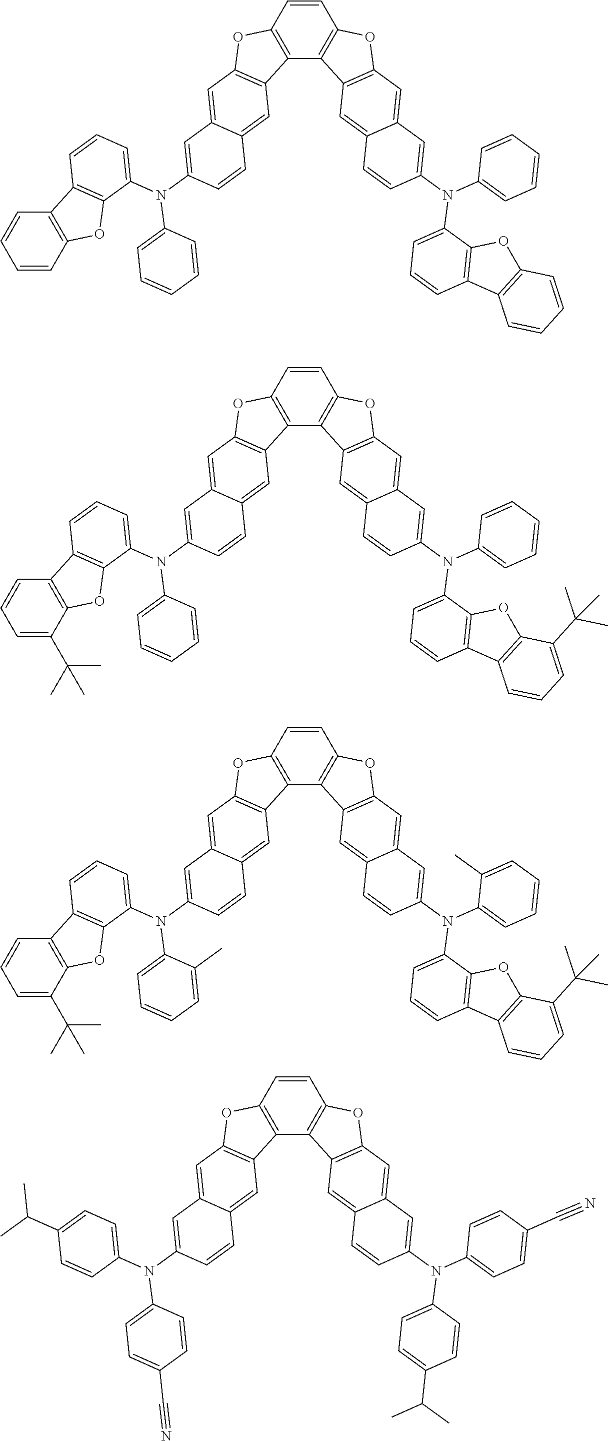

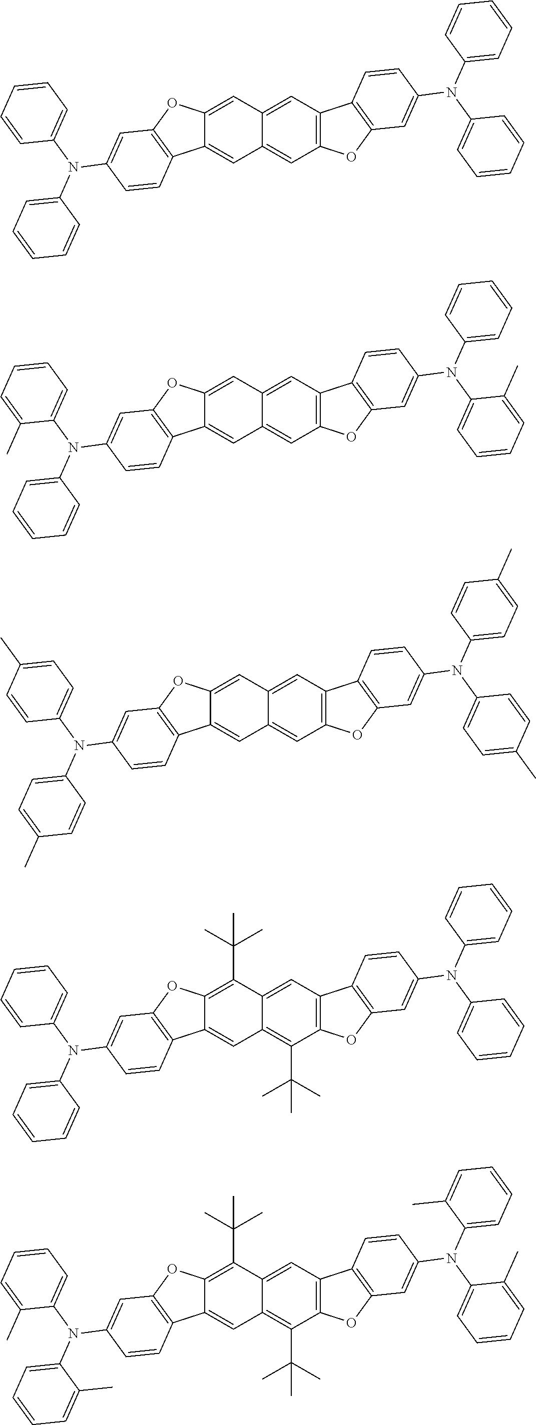

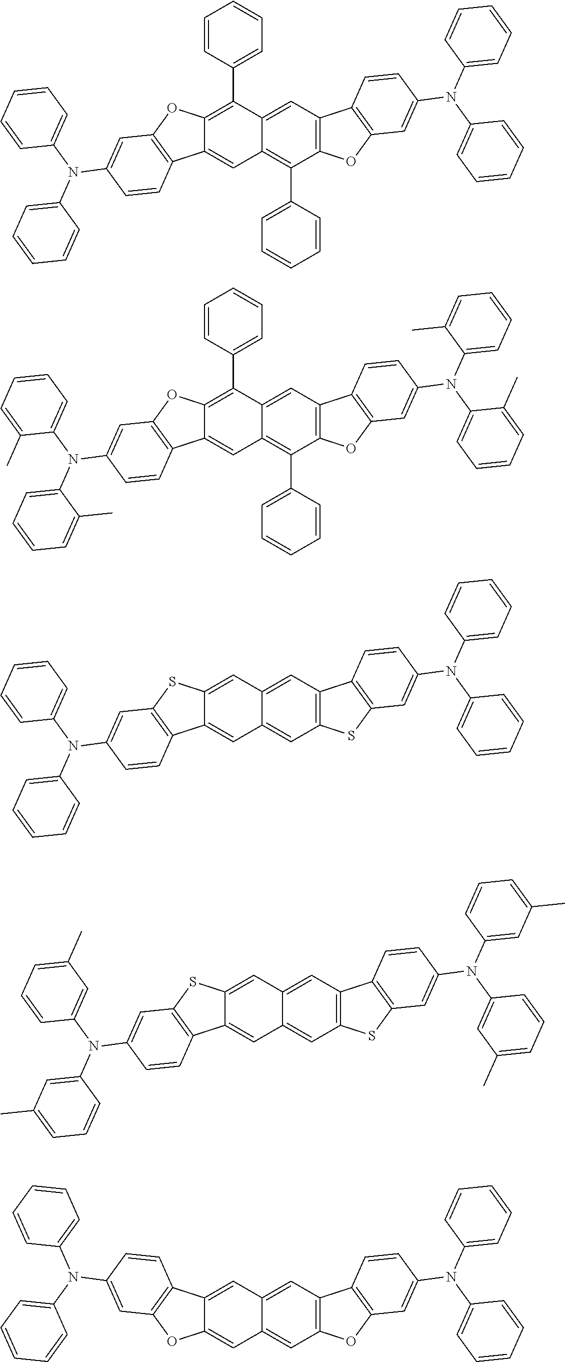

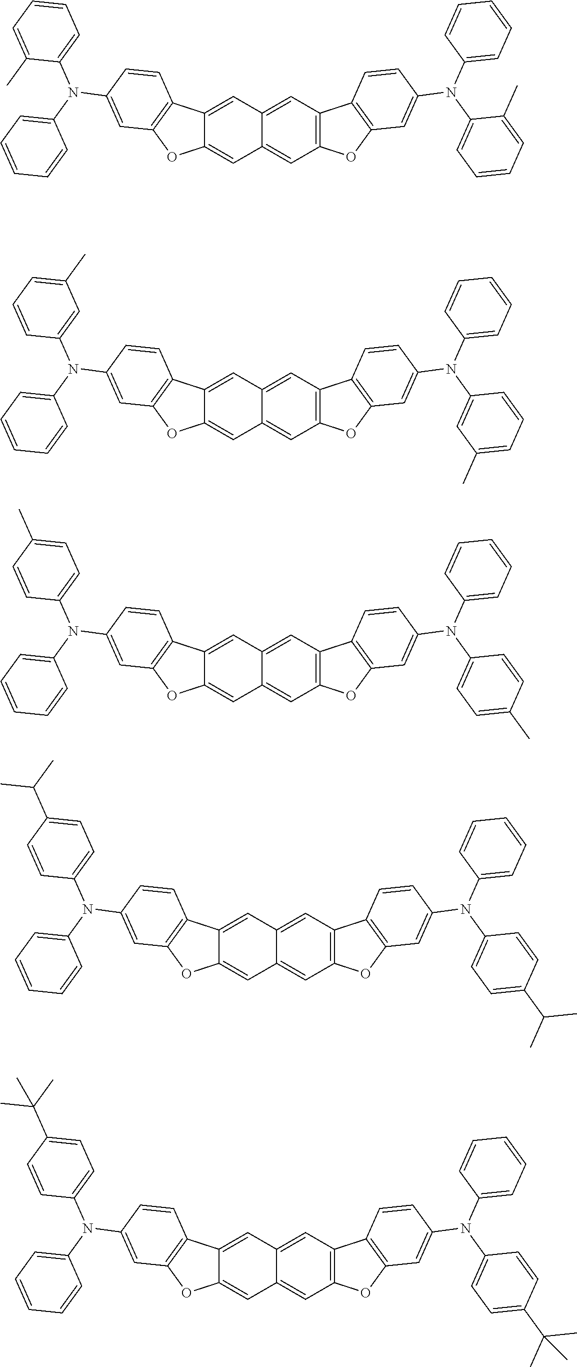

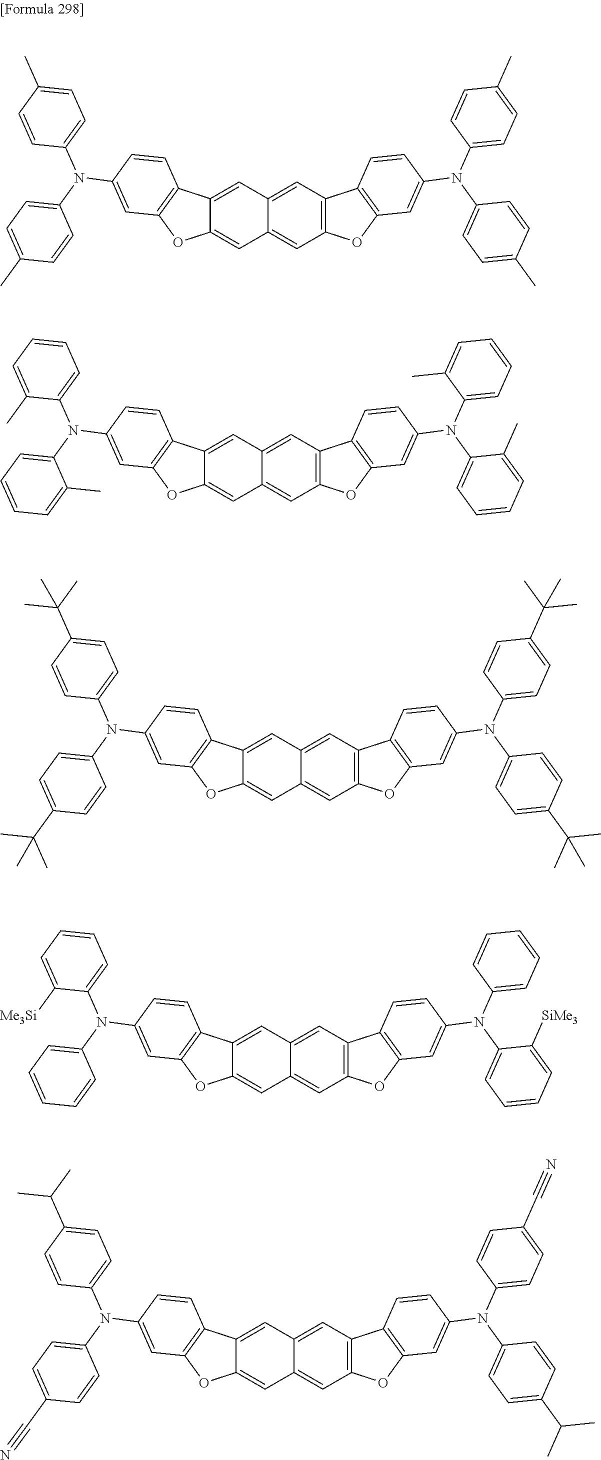

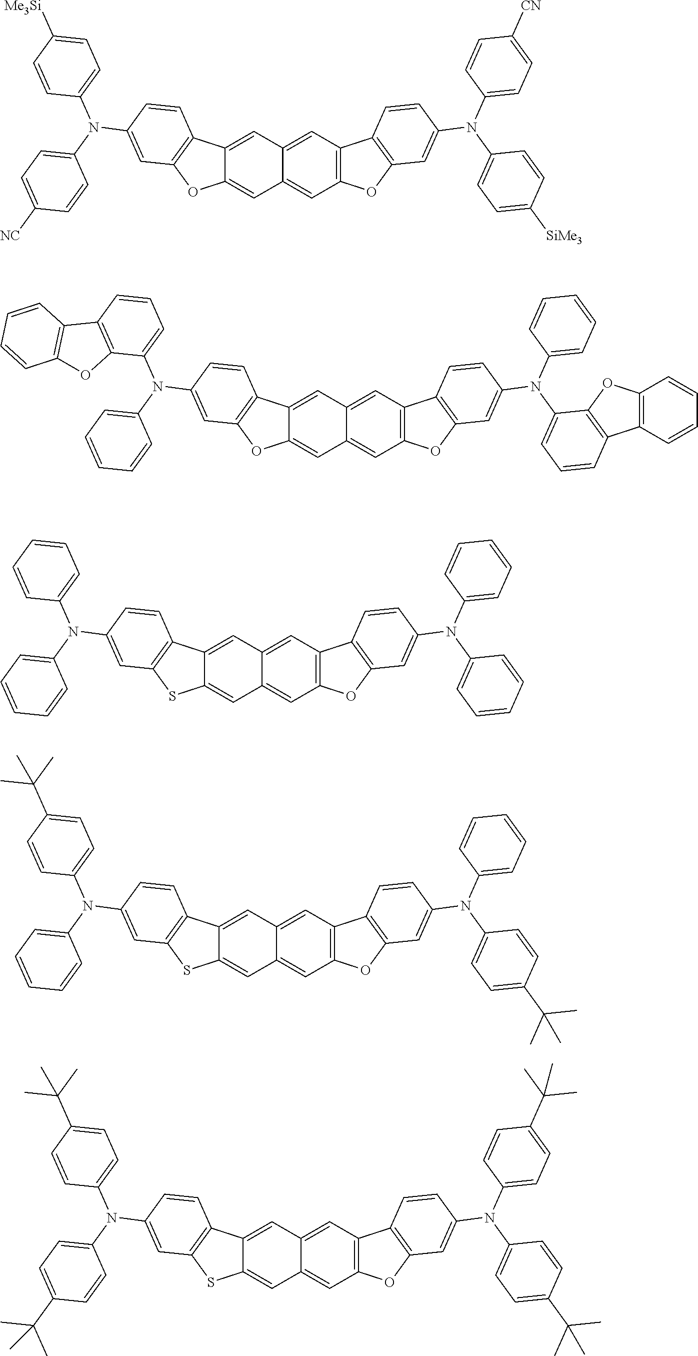

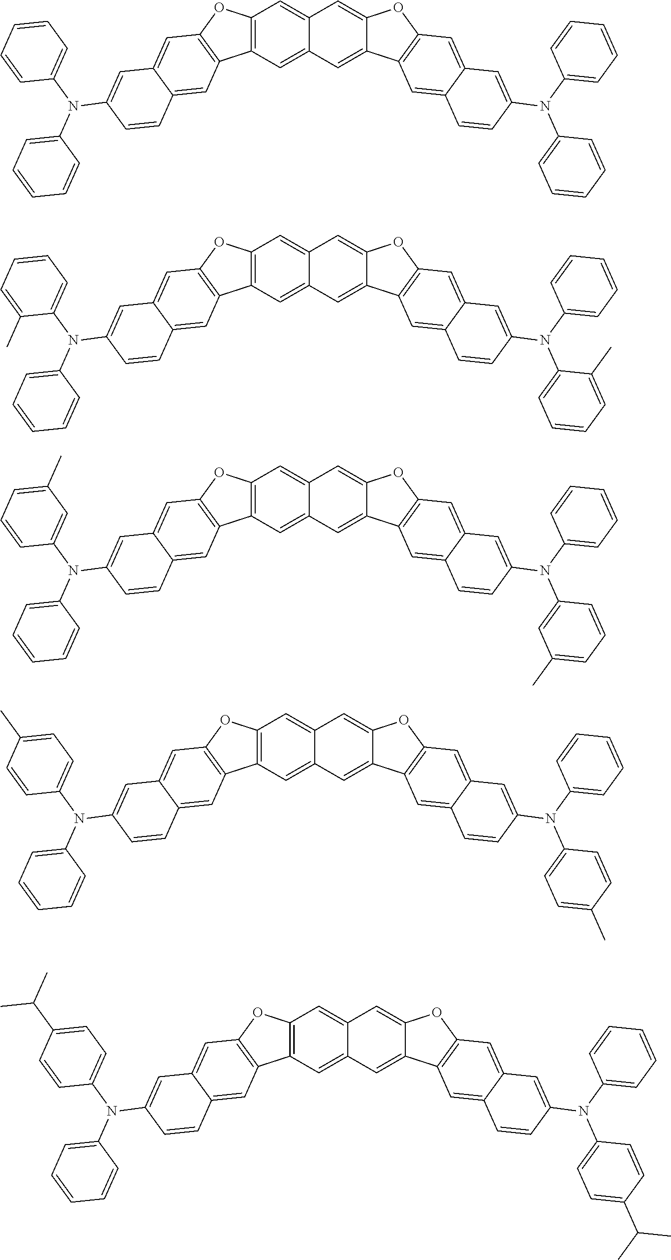

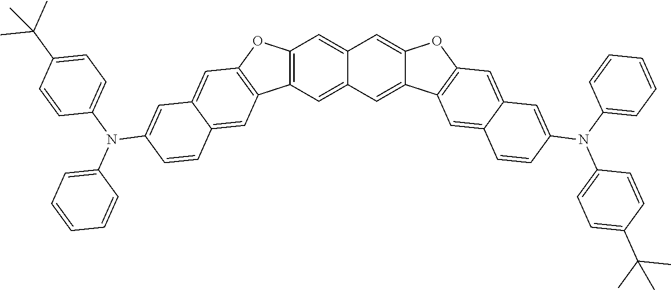 [SMILES: CC(C)(C)c1ccc(N(c2ccccc2)c2ccc3cc4c(cc3c2)oc2cc3cc5oc6cc7cc(N(c8ccccc8)c8ccc(C(C)(C)C)cc8)ccc7cc6c5cc3cc24)cc1]

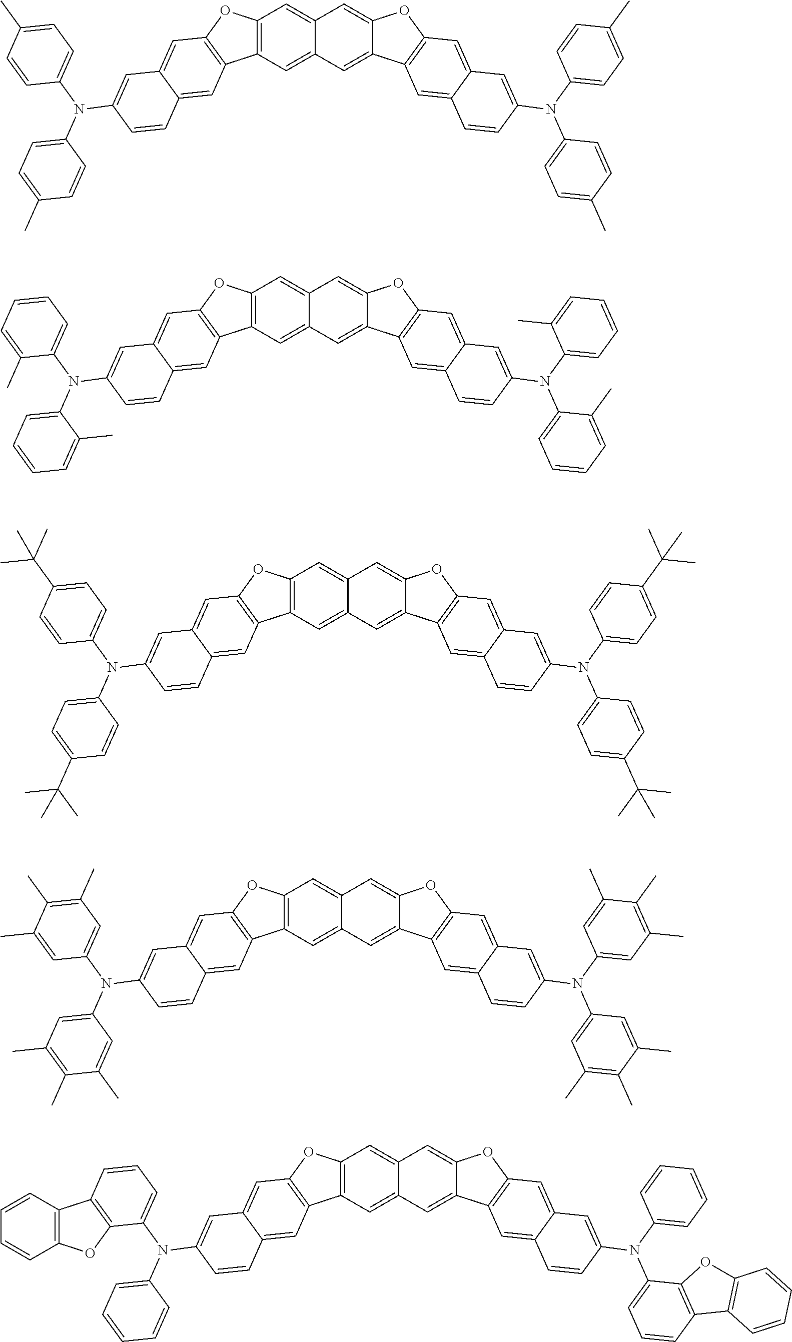

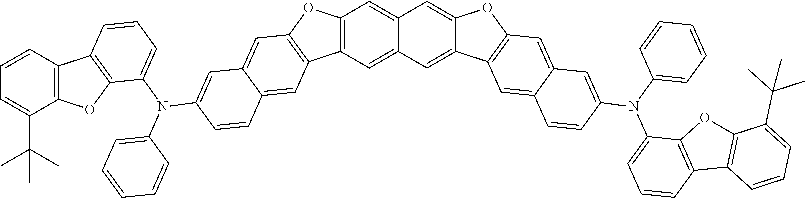 [SMILES: CC(C)(C)c1cccc2c1oc1c(N(c3ccccc3)c3ccc4cc5c(cc4c3)oc3cc4cc6oc7cc8cc(N(c9ccccc9)c9cccc%10c9oc9c(C(C)(C)C)cccc9%10)ccc8cc7c6cc4cc35)cccc12]

### Compound Represented by Formula (8)

The compound represented by the formula (8) will be described below.

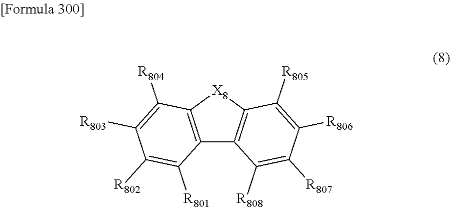

In the formula (8): at least one combination of R₈₀₁ and R₈₀₂, R₈₀₂ and R₈₀₃, or R₈₀₃ and R₈₀₄ are mutually bonded to form a divalent group represented by a formula (82) below; and
at least one combination of R₈₀₅ and R₈₀₆, R₈₀₆ and R₈₀₇, or R₈₀₇ and R₈₀₈ are mutually bonded to form a divalent group represented by a formula (83) below.

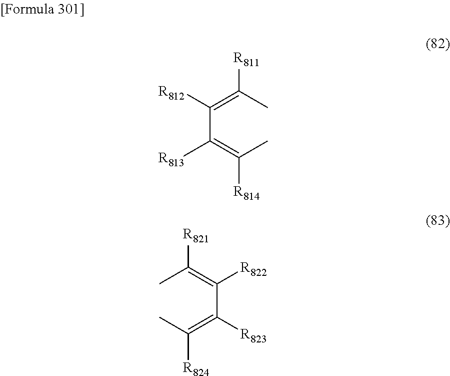

At least one of R₈₀₁ to R₈₀₄ not forming the divalent group represented by the formula (82) or R₈₁₁ to R₈₁₄ is a monovalent group represented by a formula (84) below.

At least one of R₈₀₅ to R₈₀₈ not forming the divalent group represented by the formula (83) or R₈₂₁ to R₈₂₄ is a monovalent group represented by a formula (84) below.

X₈ is an oxygen atom, a sulfur atom, or NR₈₀₉.

R₈₀₁ to R₈₀₈ not forming the divalent group represented by the formula (82) or (83) and not being the monovalent group represented by the formula (84), R₈₁₁ to R₈₁₄ and R₈₂₁ to R₈₂₄ not being the monovalent group represented by the formula (84), and R₈₀₉ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

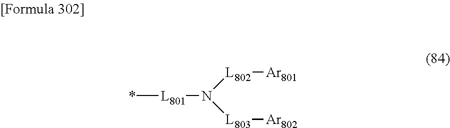

In the formula (84): Ar₈₀₁ and Ar₈₀₂ each independently represent a substituted or unsubstituted aryl group having 6 to 50 ring carbon atoms, or a substituted or unsubstituted heterocyclic group having 5 to 50 ring atoms;
L₈₀₁ to L₈₀₃ each independently represent a single bond, a substituted or unsubstituted arylene group having 6 to 30 ring carbon atoms, a substituted or unsubstituted heterocyclic group having 5 to 30 ring atoms, or a divalent linking group formed by bonding two, three or four groups selected from the group consisting of the substituted or unsubstituted arylene group having 6 to 30 ring carbon atoms and a substituted or unsubstituted divalent heterocyclic group having 5 to 30 ring atoms; and
* in the formula (84) represents a bonding position with the cyclic structure represented by the formula (8) or a bonding position to the group represented by the formula (82) or (83).

In the formula (8), the positions for the divalent group represented by the formula (82) and the divalent group represented by the formula (83) to be formed are not specifically limited but the divalent groups may be formed at any possible positions on R₈₀₁ to R₈₀₈.

In an exemplary embodiment, the compound represented by the formula (8) is represented by any one of formulae (81-1) to (81-6) below.

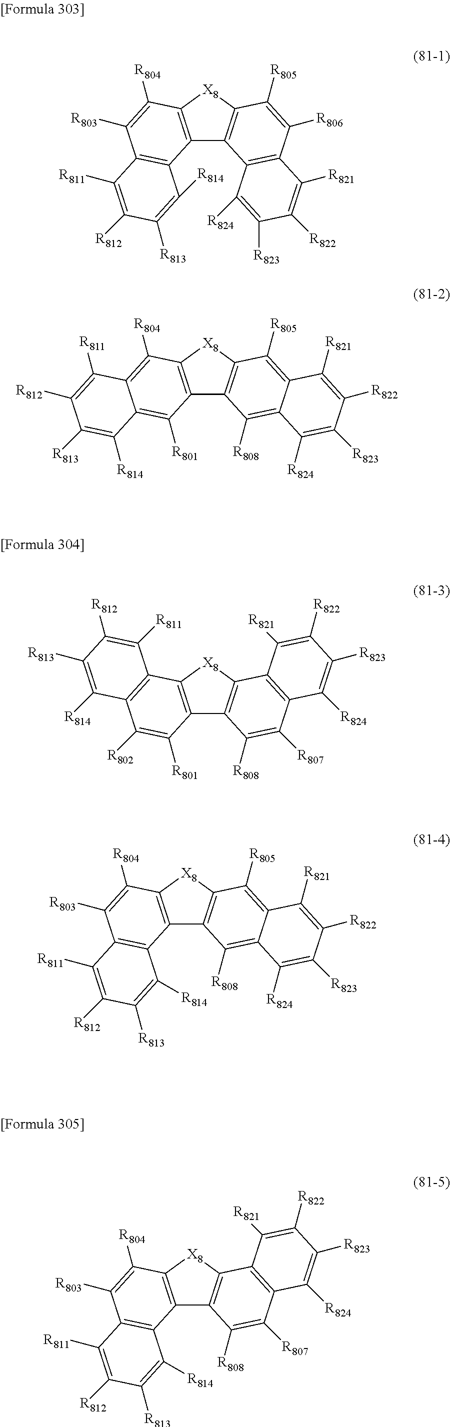

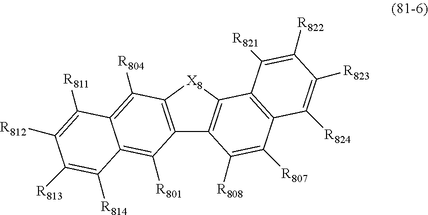

In the formulae (81-1) to (81-6):
X₈ represents the same as X₈ in the formula (8);
at least two of R₈₀₁ to R₈₂₄ are each a monovalent group represented by the formula (84); and
R₈₀₁ to R₈₂₄ that are not the monovalent group represented by the formula (84) are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, the compound represented by the formula (8) is represented by any one of formulae (81-7) to (81-18) below.

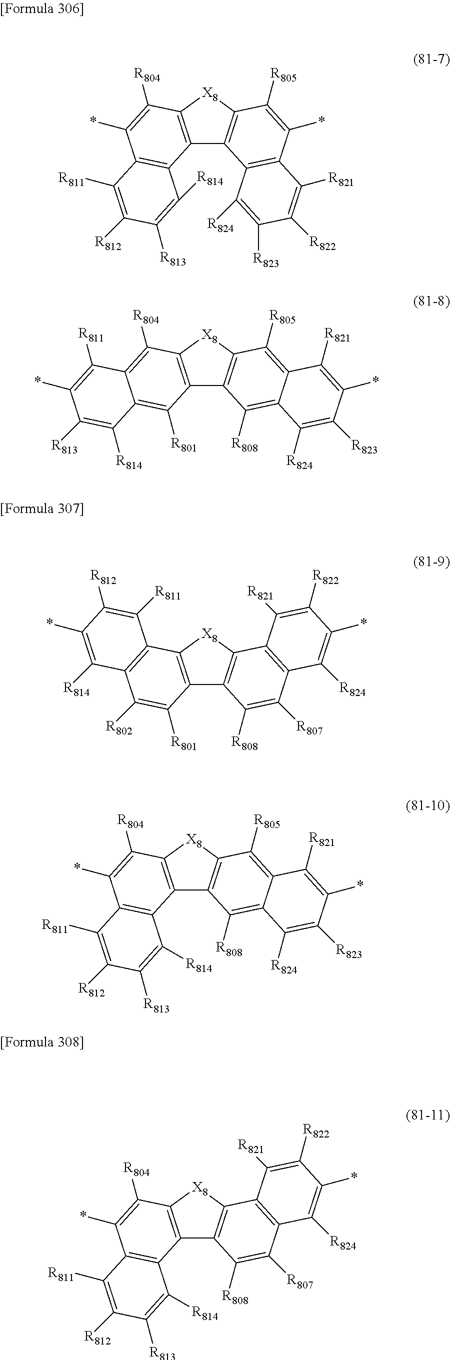

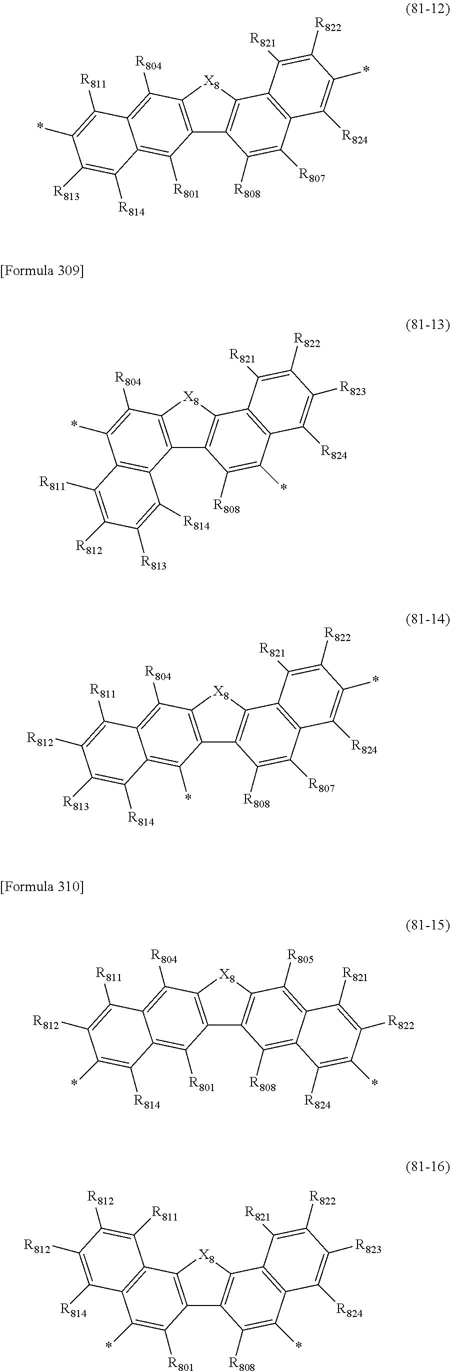

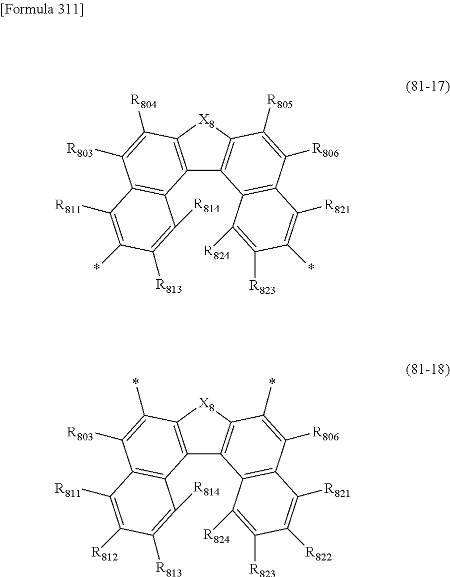

In the formulae (81-7) to (81-18):
X₈ represents the same as X₈ in the formula (8);
* is a single bond to be bonded with the monovalent group represented by the formula (84);
R₈₀₁ to R₈₂₄ each independently represent the same as R₈₀₁ to R₈₂₄ in the formulae (81-1) to (81-6) that are not the monovalent group represented by the formula (84).

R₈₀₁ to R₈₀₈ not forming the divalent group represented by the formula (82) or (83) and not being the monovalent group represented by the formula (84), and R₈₁₁ to R₈₁₄ and R₈₂₁ to R₈₂₄ not being the monovalent group represented by the formula (84) are each independently preferably a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

The monovalent group represented by the formula (84) is preferably represented by a formula (85) or (86) below.

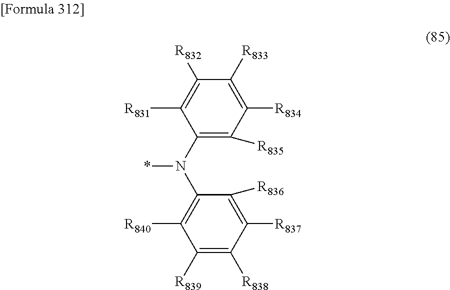

In the formula (85): R₈₃₁ to R₈₄₀ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
* in the formula (85) represents the same as * in the formula (84).

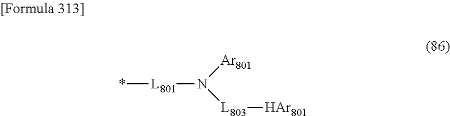

In the formula (86): Ar₈₀₁, L₈₀₁, and L₈₀₃ represent the same as Ar₈₀₁, L₈₀₁, and L₈₀₃ in the formula (84); and
HAr₈₀₁ is a moiety represented by a formula (87) below.

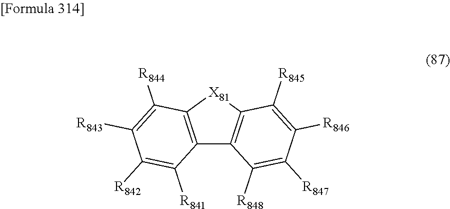

In the formula (87):
X₈₁ represents an oxygen atom or a sulfur atom;
one of R₈₄₁ to R₈₄₈ is a single bond with L₈₀₃; and
R₈₄₁ to R₈₄₈ not being the single bond are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

### Specific Examples of Compound Represented by Formula (8)

Specific examples of the compound represented by the formula (8) include compounds shown below as well as the compounds disclosed in International Publication No. WO 2014/104144.

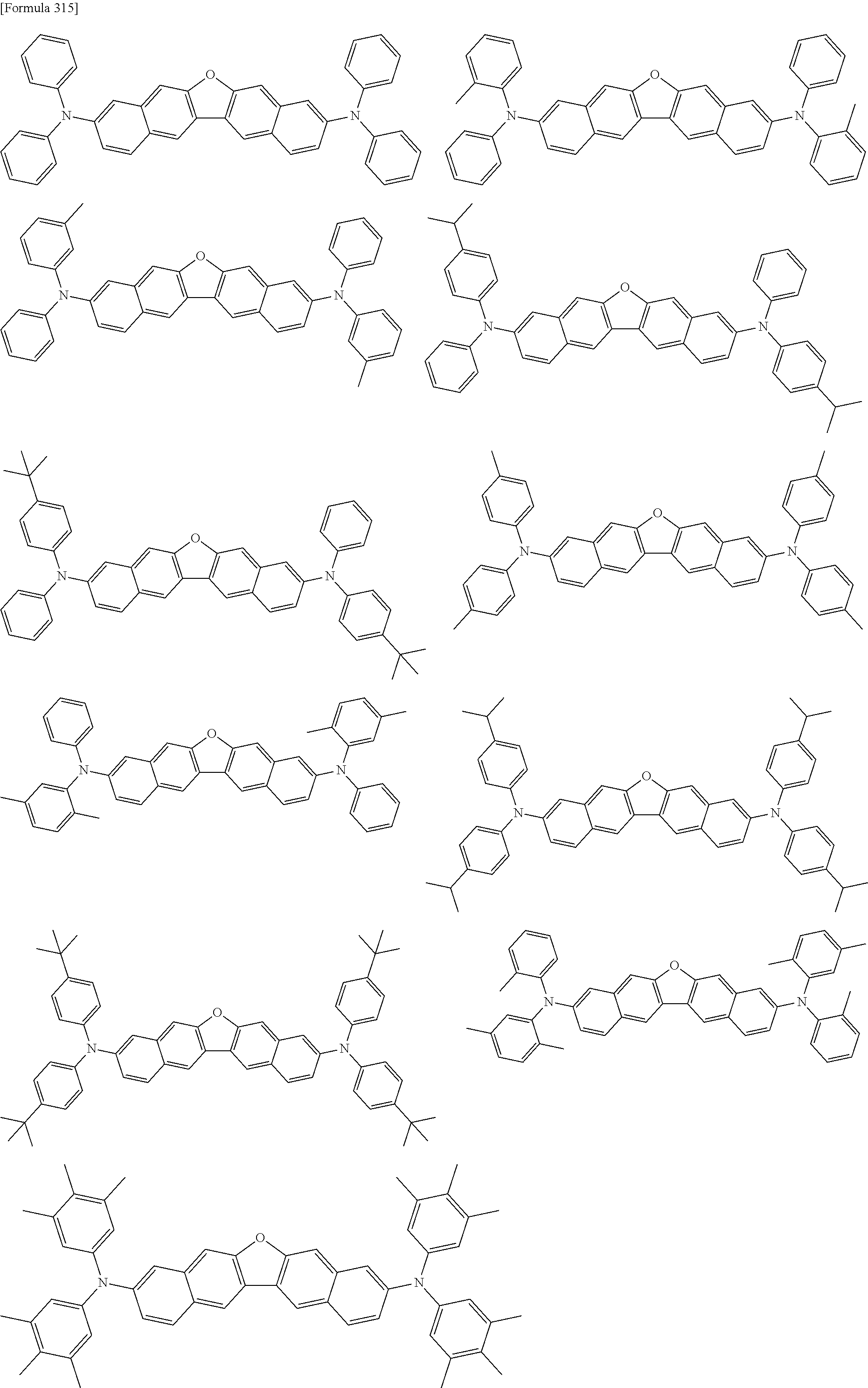

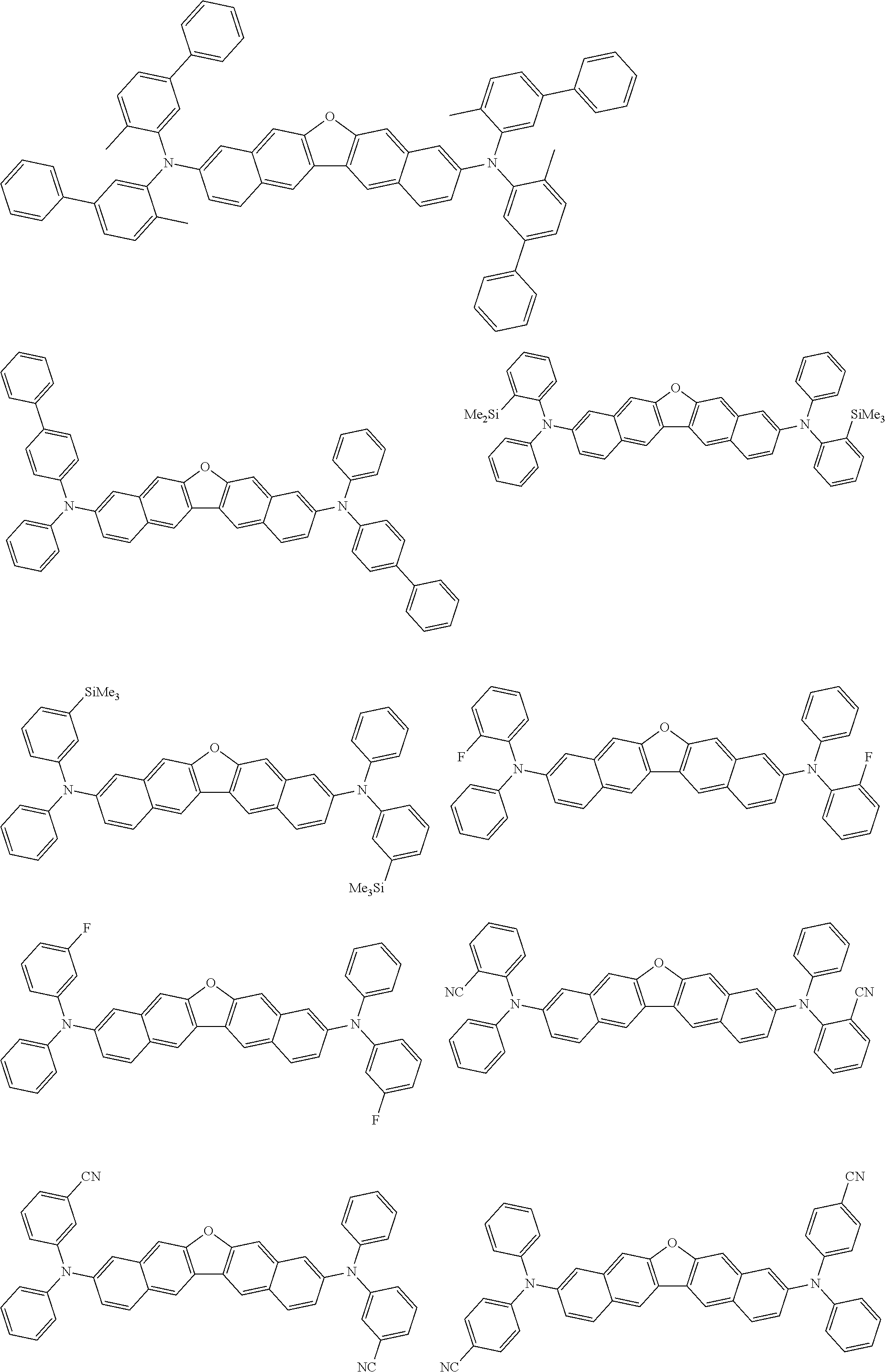

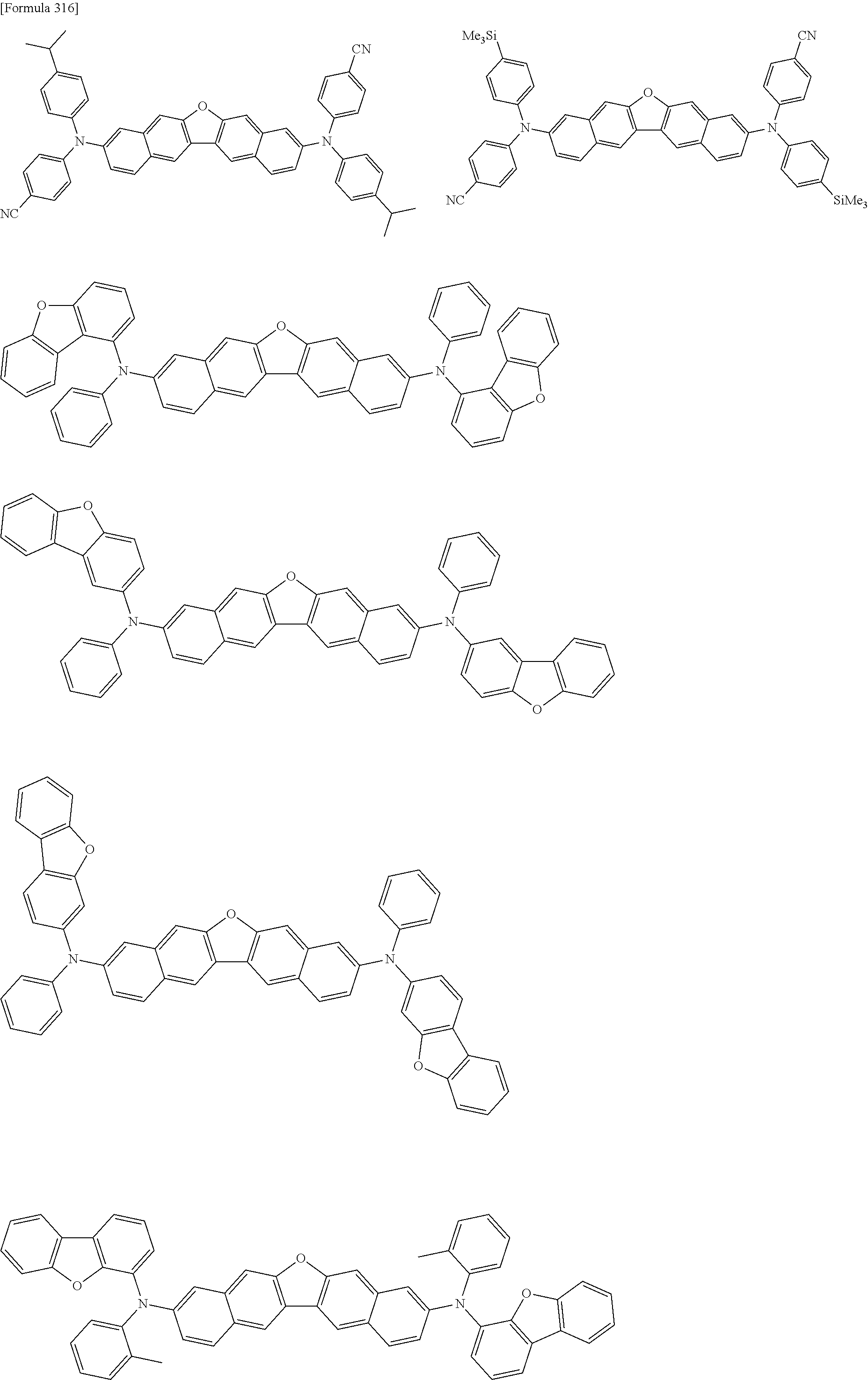

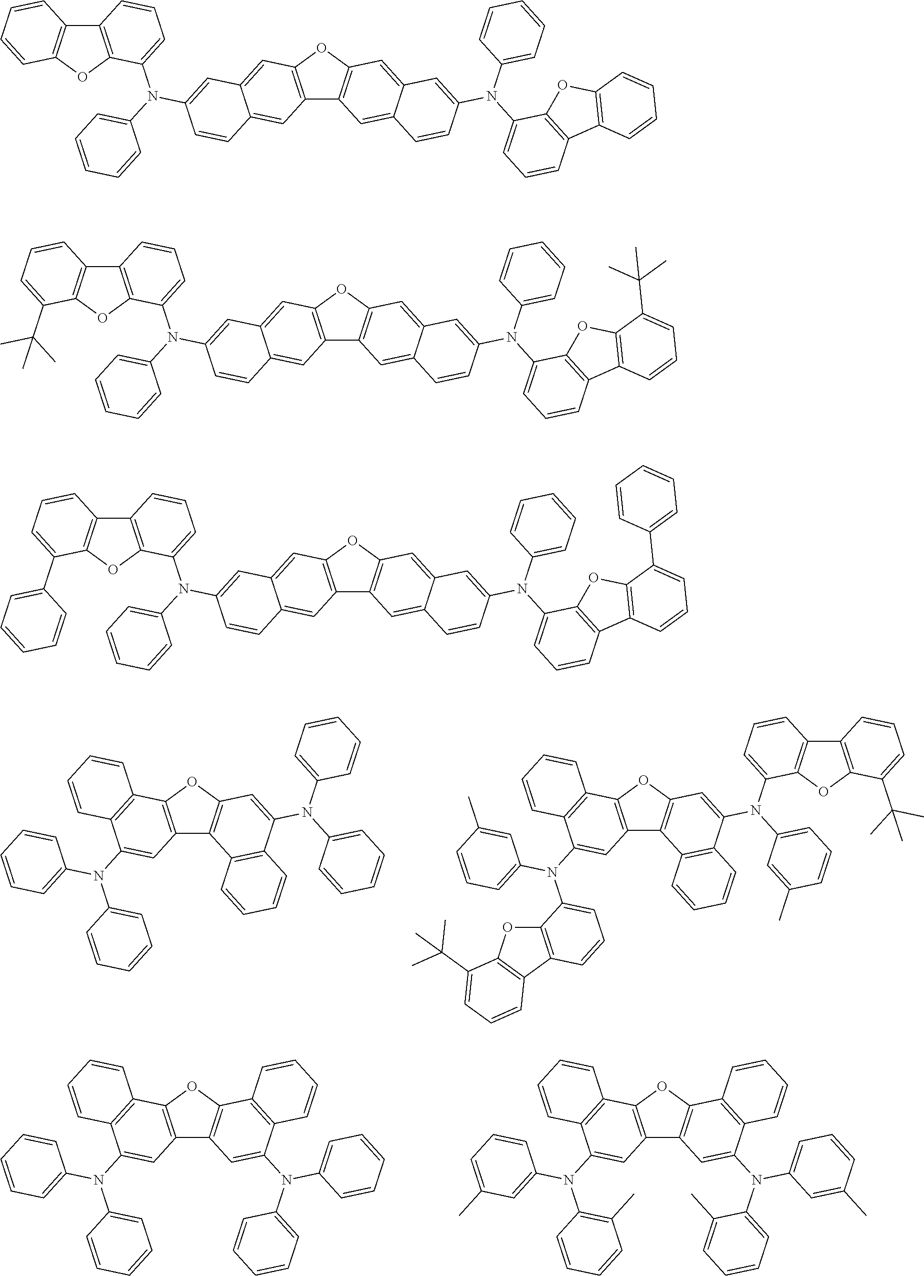

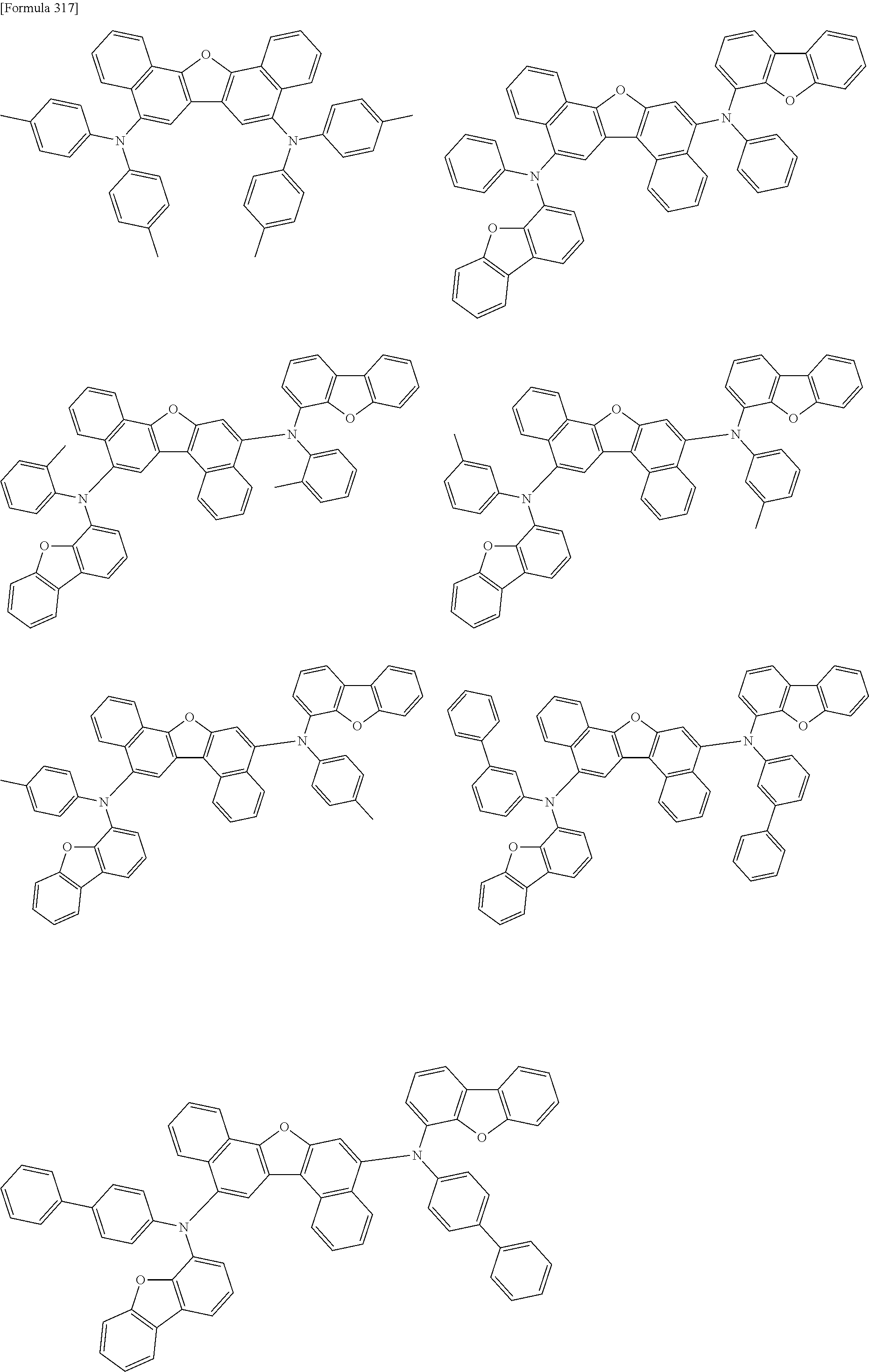

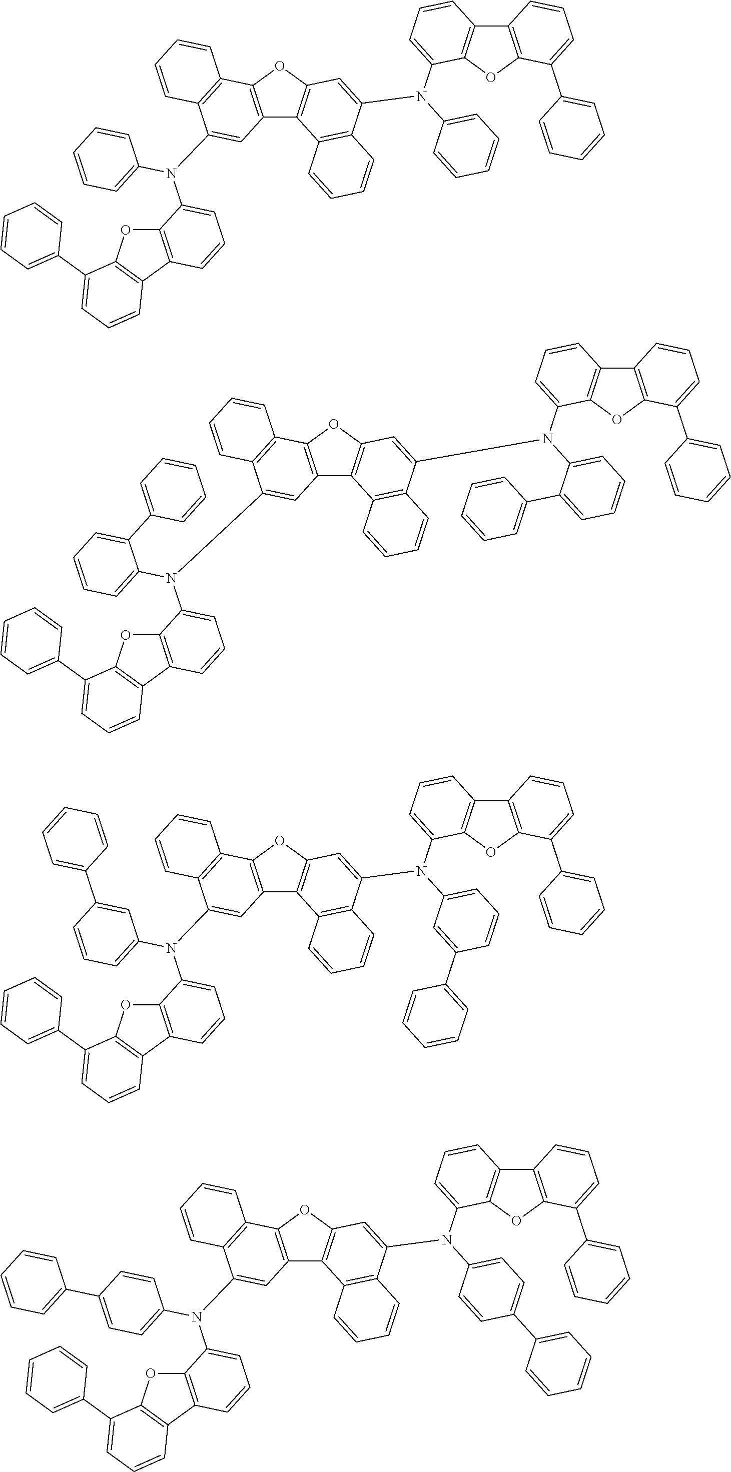

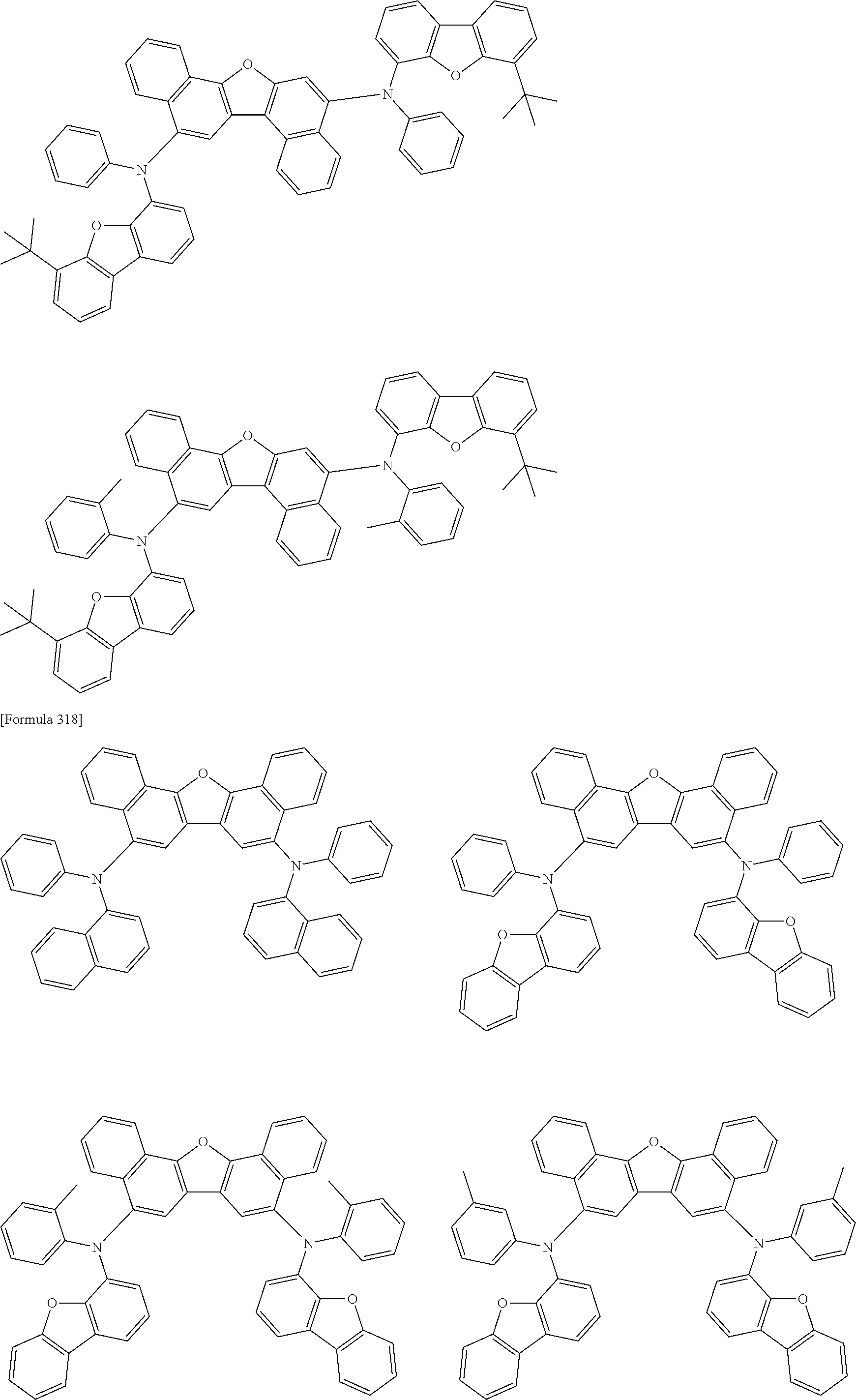

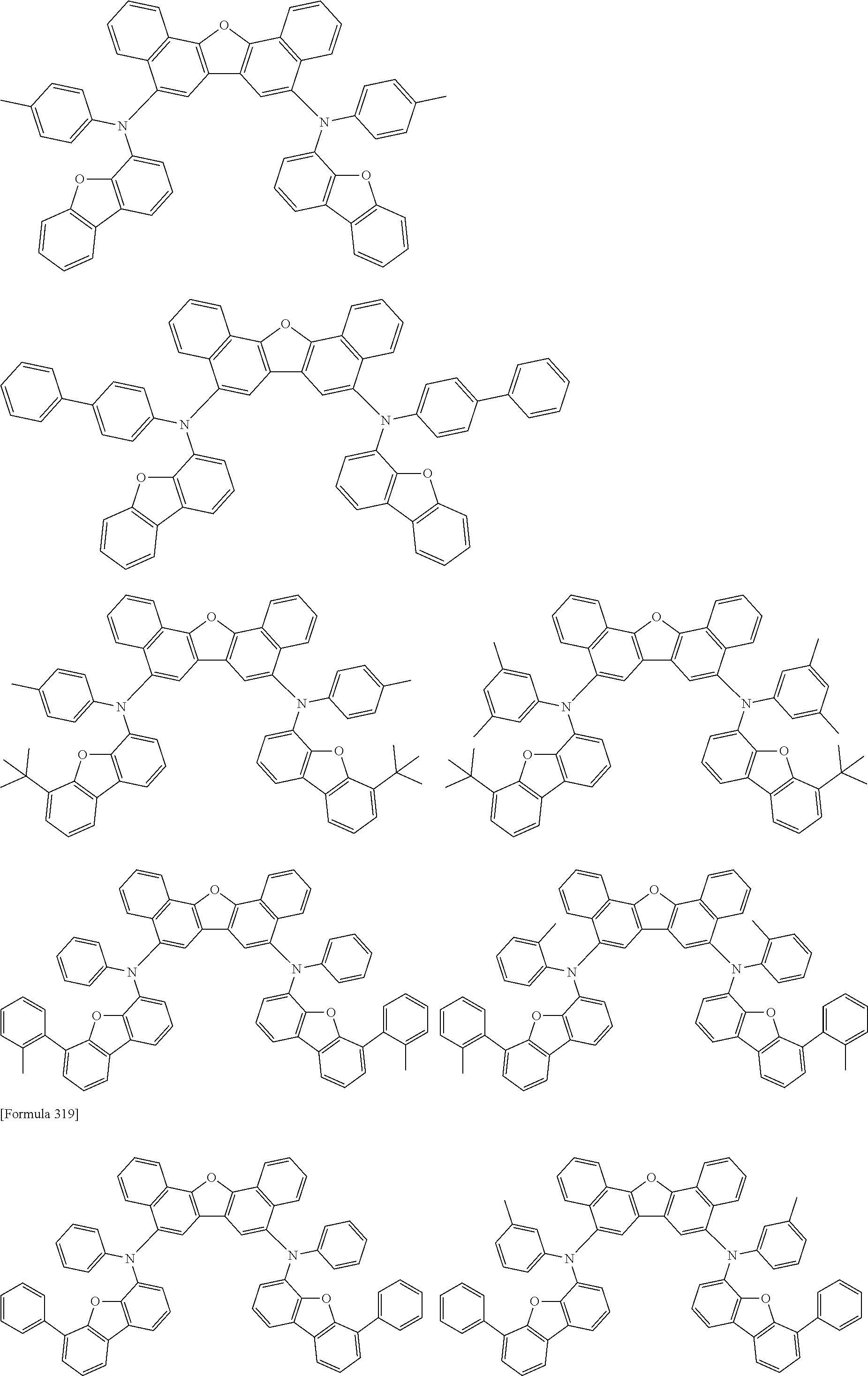

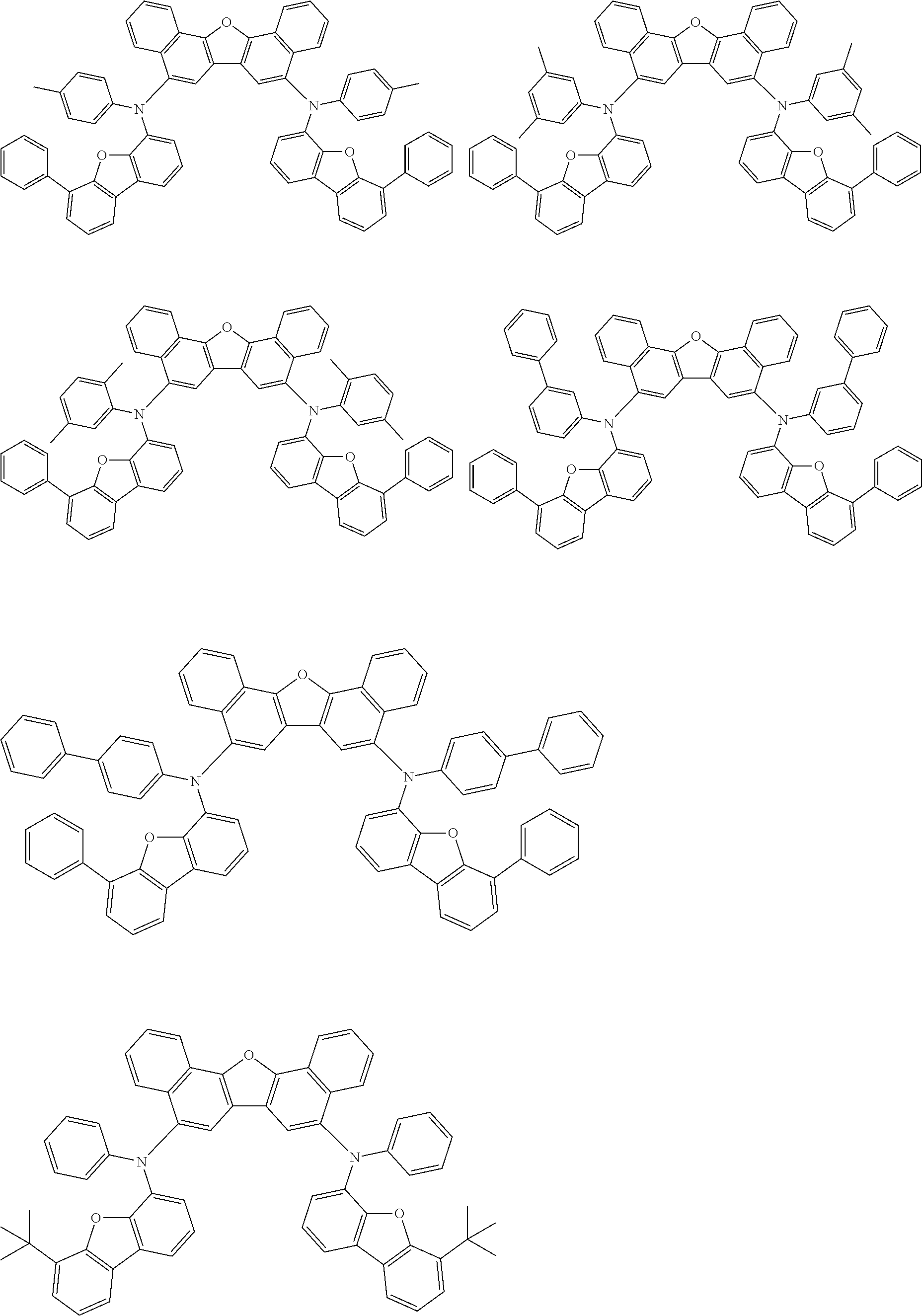

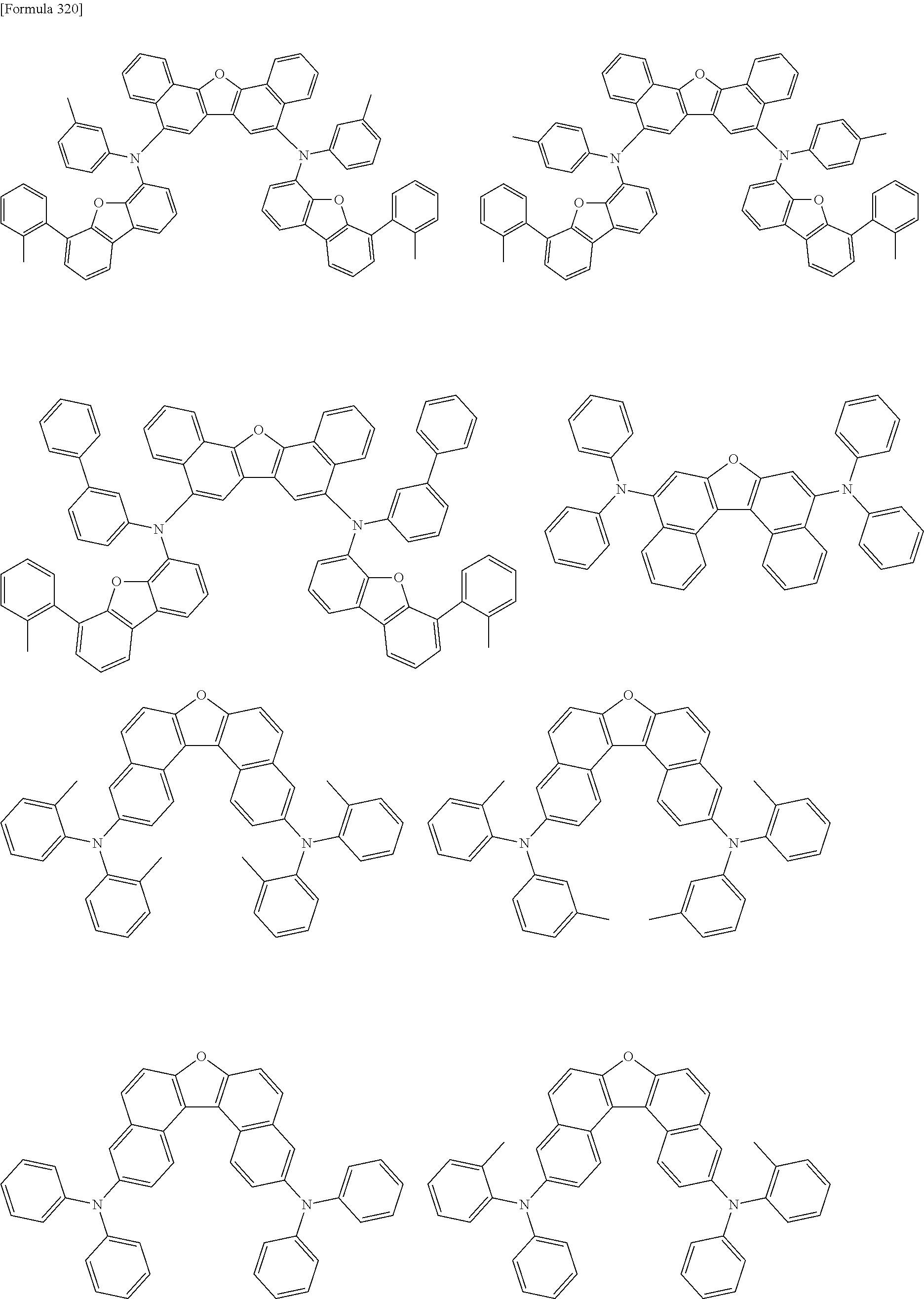

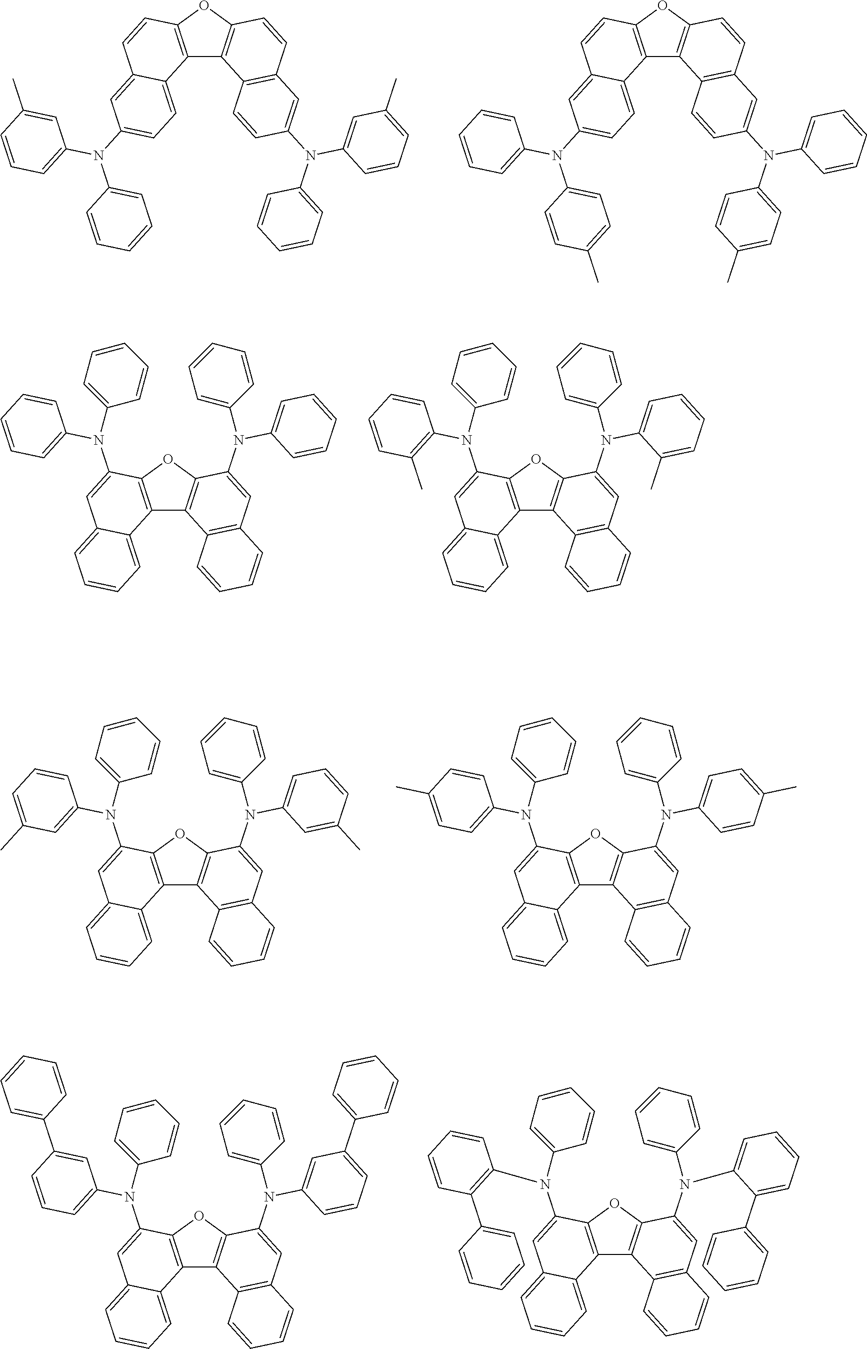

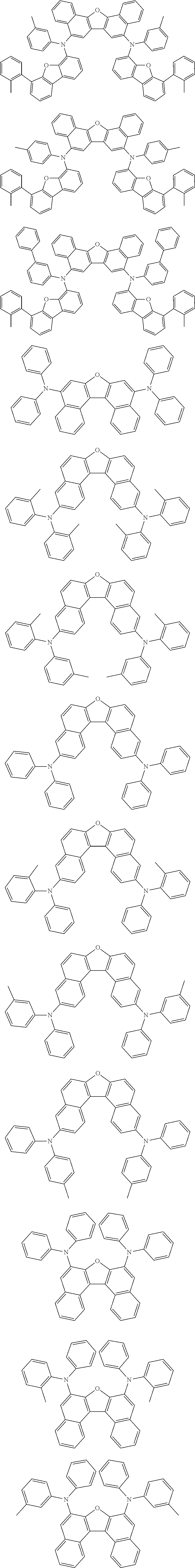

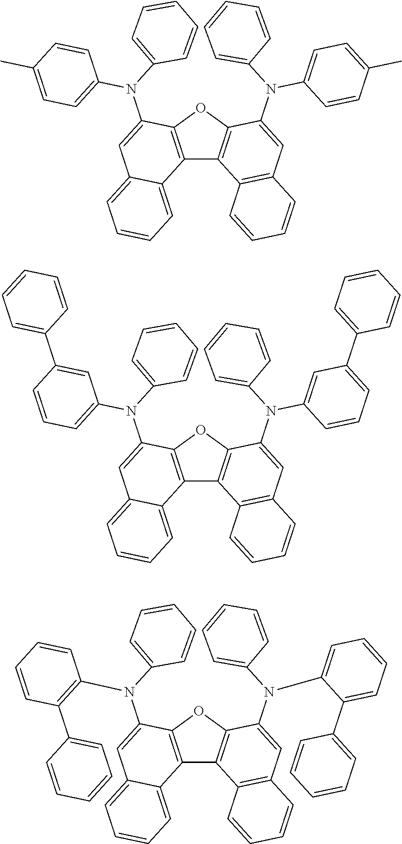

### Compound Represented by Formula (9)

The compound represented by the formula (9) will be described below.

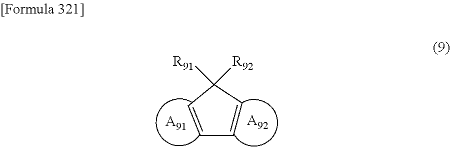

In the formula (9): A₉₁ ring and A₉₂ ring are each independently a substituted or unsubstituted aromatic hydrocarbon ring having 6 to 50 ring carbon atoms or a substituted or unsubstituted heterocycle having 5 to 50 ring atoms; and
at least one of the A₉₁ ring or the A₉₂ ring is bonded with * in a moiety represented by a formula (92) below.

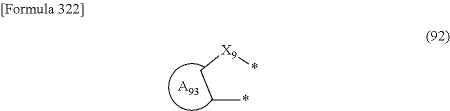

In the formula (92): A₉₃ ring is a substituted or unsubstituted aromatic hydrocarbon ring having 6 to 50 ring carbon atoms or a substituted or unsubstituted heterocycle having 5 to 50 ring atoms;
X₉ is NR₉₃, C(R₉₄)(R₉₅), Si(R₉₆)(R₉₇), Ge(R₉₈)(R₉₉), an oxygen atom, a sulfur atom, or a selenium atom;
R₉₁ and R₉₂ are mutually bonded to form a substituted or unsubstituted monocyclic ring, mutually bonded to form a substituted or unsubstituted fused ring, or not mutually bonded; and R₉₁ and R₉₂ not forming the monocyclic ring and not forming the fused ring, and R₉₃ to R₉₉ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

At least one ring selected from the group consisting of the A₉₁ ring and the A₉₂ ring is bonded to a bond * of the moiety represented by the formula (92). In other words, the ring carbon atoms of the aromatic hydrocarbon ring or the ring atoms of the heterocycle of the A₉₁ ring in an exemplary embodiment are bonded to the bonds * in the moiety represented by the formula (92). Further, the ring carbon atoms of the aromatic hydrocarbon ring or the ring atoms of the heterocycle of the A₉₂ ring in an exemplary embodiment are bonded to the bonds * in the moiety represented by the formula (92).

In an exemplary embodiment, the group represented by a formula (93) below is bonded to one or both of the A₉₁ ring and A₉₂ ring.

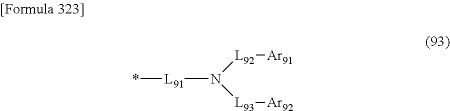

In the formula (93): Ar₉₁ and Ar₉₂ are each independently a substituted or unsubstituted aryl group having 6 to 50 ring carbon atoms, or a substituted or unsubstituted heterocyclic group having 5 to 50 ring atoms;
L₉₁ to L₉₃ each independently represent a single bond, a substituted or unsubstituted arylene group having 6 to 30 ring carbon atoms, a substituted or unsubstituted heterocyclic group having 5 to 30 ring atoms, or a divalent linking group formed by bonding two, three or four groups selected from the group consisting of the substituted or unsubstituted arylene group having 6 to 30 ring carbon atoms and a substituted or unsubstituted divalent heterocyclic group having 5 to 30 ring atoms; and
* in the formula (93) represents a bonding position with one of the A₉₁ ring and the A₉₂ ring.

In an exemplary embodiment, in addition to the A₉₁ ring, the ring carbon atoms of the aromatic hydrocarbon ring or the ring atoms of the heterocycle of the A₉₂ ring are bonded to * in the moiety represented by the formula (92). In this case, the moieties represented by the formula (92) are mutually the same or different.

In an exemplary embodiment, R₉₁ and R₉₂ are each independently a substituted or unsubstituted aryl group having 6 to 50 ring carbon atoms.

In an exemplary embodiment, R₉₁ and R₉₂ are mutually bonded to form a fluorene structure.

In an exemplary embodiment, the rings A₉₁ and A₉₂ are each independently a substituted or unsubstituted aromatic hydrocarbon ring having 6 to 50 ring carbon atoms, example of which is a substituted or unsubstituted benzene ring.

In an exemplary embodiment, the ring A₉₃ is a substituted or unsubstituted aromatic hydrocarbon ring having 6 to 50 ring carbon atoms, example of which is a substituted or unsubstituted benzene ring.

In an exemplary embodiment, X₉ is an oxygen atom or a sulfur atom.

### Specific Examples of Compound Represented by Formula (9)

Specific examples of the compound represented by the formula (9) include compounds shown below.

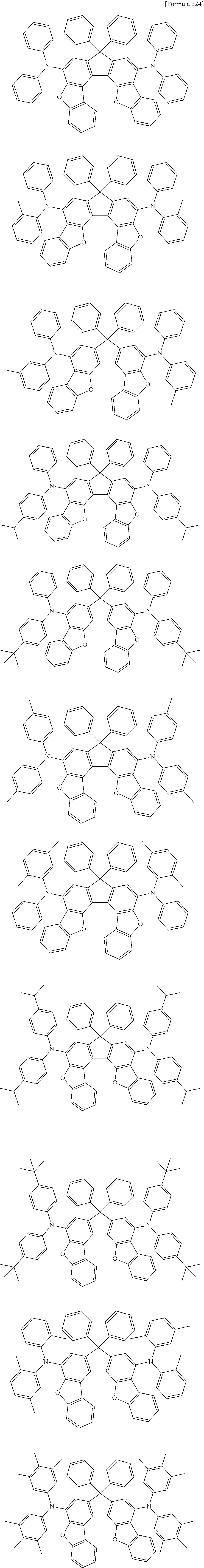

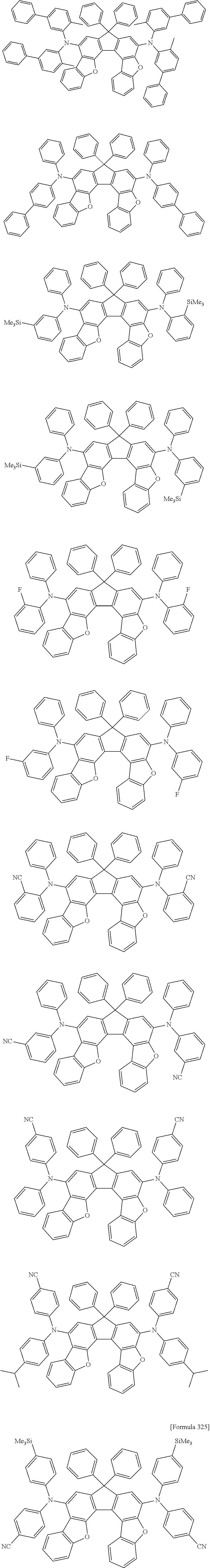

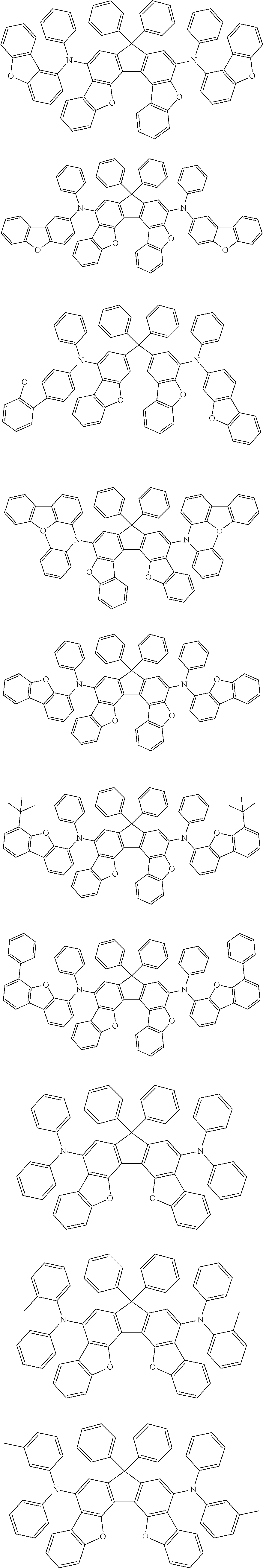

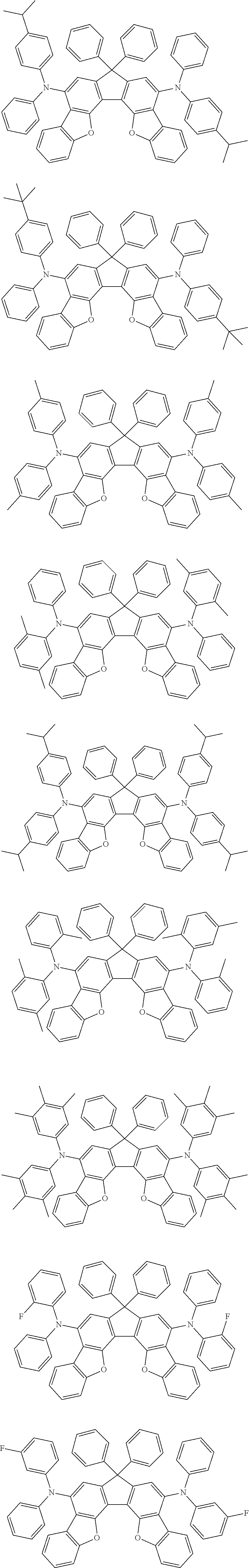

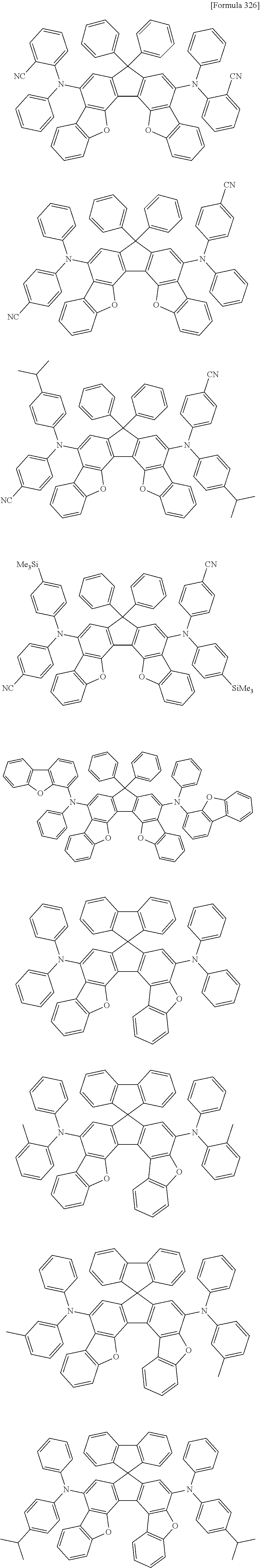

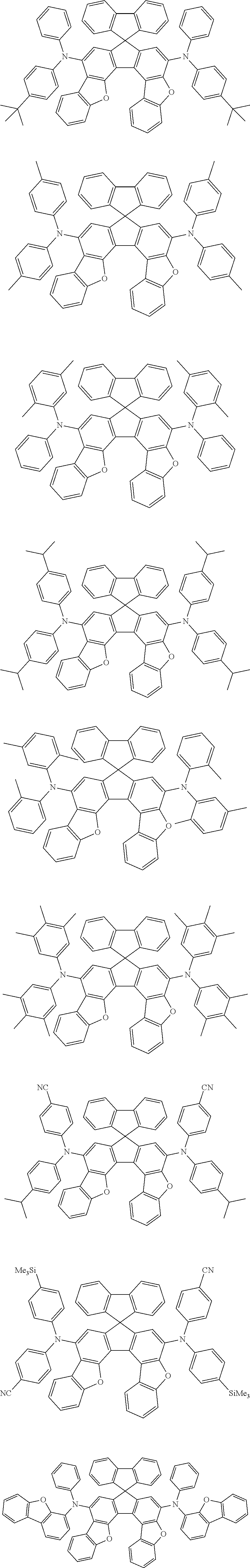

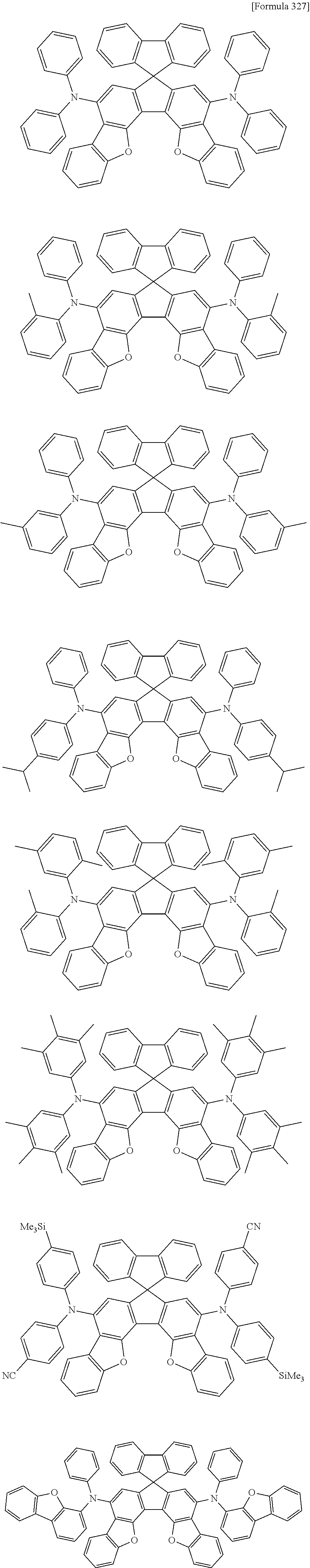

### Compound Represented by Formula (10)

The compound represented by the formula (10) will be described below.

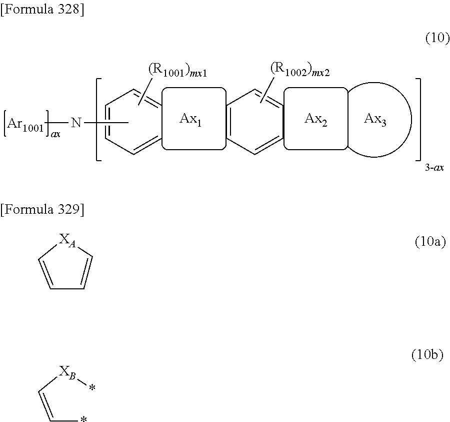

In the formula (10):
Ax₁ ring is a ring represented by the formula (10a) and fused with any positions of adjacent rings;
Ax₂ ring is a ring represented by the formula (10b) and fused with any positions of adjacent rings;
two * in the formula (10b) are bonded to any position of Ax₃ ring;
X_{A} and X_{B} are each independently C(R₁₀₀₃)(R₁₀₀₄), Si(R₁₀₀₅)(R₁₀₀₆), an oxygen atom or a sulfur atom;
Ax₃ ring is a substituted or unsubstituted aromatic hydrocarbon ring having 6 to 50 ring carbon atoms or a substituted or unsubstituted heterocycle having 5 to 50 ring atoms;
Ar₁₀₀₁ is a substituted or unsubstituted aryl group having 6 to 50 ring carbon atoms, or a substituted or unsubstituted heterocyclic group having 5 to 50 ring atoms;
R₁₀₀₁ to R₁₀₀₆ each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
mx1 is 3, mx2 is 2;
a plurality of R₁₀₀₁ are mutually the same or different;
a plurality of R₁₀₀₂ are mutually the same or different;
ax is 0, 1, or 2;
when ax is 0 or 1, the structures enclosed by brackets indicated by “3-ax” are mutually the same or different; and
when ax is 2, a plurality of Ar₁₀₀₁ are mutually the same or different.

In an exemplary embodiment, Ar₁₀₀₁ is a substituted or unsubstituted aryl group having 6 to 50 ring carbon atoms.

In an exemplary embodiment, Ax₃ ring is a substituted or unsubstituted aromatic hydrocarbon ring having 6 to 50 ring carbon atoms, example of which is a substituted or unsubstituted benzene ring, a substituted or unsubstituted naphthalene ring, or a substituted or unsubstituted anthracene ring.

In an exemplary embodiment, R₁₀₀₃ and R₁₀₀₄ are each independently a substituted or unsubstituted alkyl group having 1 to 50 carbon atoms.

In an exemplary embodiment, ax is 1.

### Specific Examples of Compound Represented by Formula (10)

Specific examples of the compound represented by the formula (10) include compounds shown below.

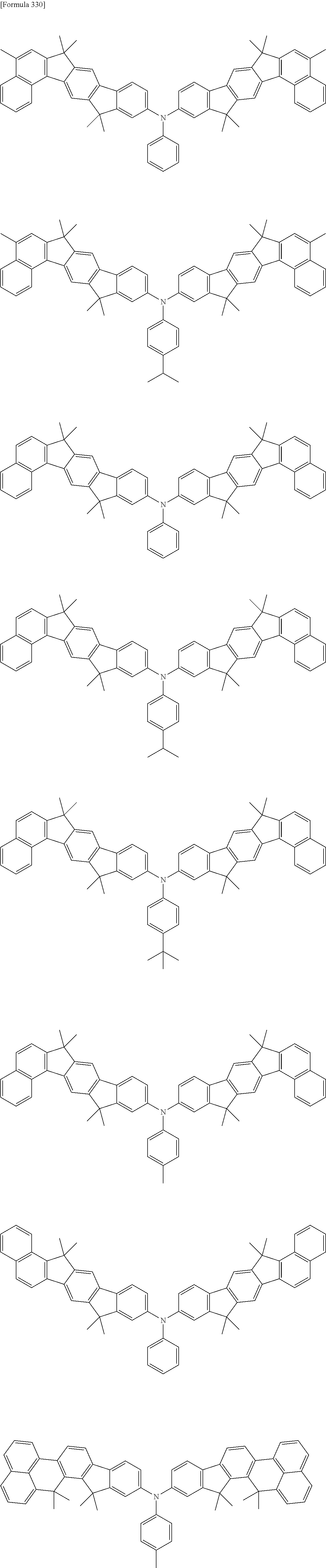

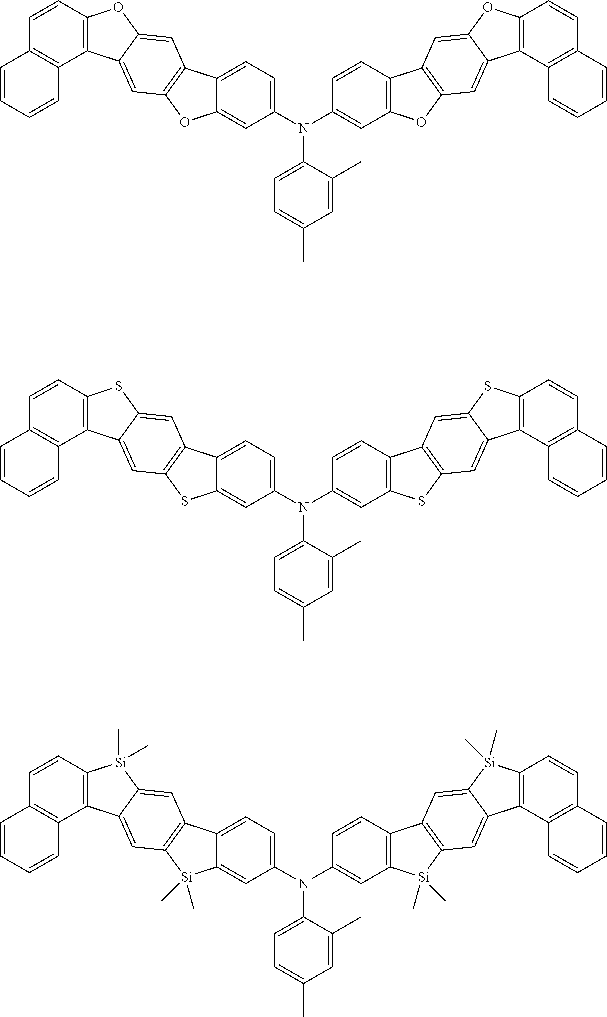 [SMILES: Cc1ccc(N(c2ccc3c(c2)oc2cc4c(cc23)oc2ccc3ccccc3c24)c2ccc3c(c2)oc2cc4c(cc23)oc2ccc3ccccc3c24)c(C)c1 ; Cc1ccc(N(c2ccc3c(c2)sc2cc4c(cc23)sc2ccc3ccccc3c24)c2ccc3c(c2)sc2cc4c(cc23)sc2ccc3ccccc3c24)c(C)c1 ; Cc1ccc(N(c2ccc3c(c2)[Si](C)(C)c2cc4c(cc2-3)[Si](C)(C)c2ccc3ccccc3c2-4)c2ccc3c(c2)[Si](C)(C)c2cc4c(cc2-3)[Si](C)(C)c2ccc3ccccc3c2-4)c(C)c1]

In an exemplary embodiment, the emitting layer contains, as at least one of the third compound or the fourth compound, at least one compound selected from the group consisting of the compound represented by the formula (4), the compound represented by the formula (5), the compound represented by the formula (7), the compound represented by the formula (8), the compound represented by the formula (9), and a compound represented by a formula (63a) below.

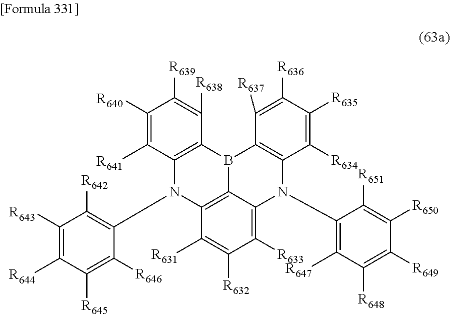

In the formula (63a): R₆₃₁ is bonded with R₆₄₆ to form a substituted or unsubstituted heterocycle or does not form a substituted or unsubstituted heterocycle;
R₆₃₃ is bonded with R₆₄₇ to form a substituted or unsubstituted heterocycle or does not form a substituted or unsubstituted heterocycle;
R₆₃₄ is bonded with R₆₅₁ to form a substituted or unsubstituted heterocycle or does not form a substituted or unsubstituted heterocycle;
R₆₄₁ is bonded with R₆₄₂ to form a substituted or unsubstituted heterocycle or does not form a substituted or unsubstituted heterocycle;
at least one combination of adjacent two or more of R₆₃₁ to R₆₅₁ are mutually bonded to form a substituted or unsubstituted monocyclic ring, mutually bonded to form a substituted or unsubstituted fused ring, or not mutually bonded;
R₆₃₁ to R₆₅₁ not forming the substituted or unsubstituted heterocycle, not forming the monocyclic ring, and not forming the fused ring are each independently a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and
at least one of R₆₃₁ to R₆₅₁ not forming the substituted or unsubstituted heterocycle, not forming the monocyclic ring, and not forming the fused ring are a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, the compound represented by the formula (4) is the compound represented by the formula (41-3), the formula (41-4) or the formula (41-5), the A1 ring in the formula (41-5) being a substituted or unsubstituted fused aromatic hydrocarbon ring having 10 to 50 ring carbon atoms, or a substituted or unsubstituted fused heterocycle having 8 to 50 ring atoms.

In an exemplary embodiment, the substituted or unsubstituted fused aromatic hydrocarbon ring having 10 to 50 ring carbon atoms in the formulae (41-3), (41-4) and (41-5) is a substituted or unsubstituted naphthalene ring, a substituted or unsubstituted anthracene ring, or a substituted or unsubstituted fluorene ring; and
the substituted or unsubstituted fused heterocycle having 8 to 50 ring atoms is a substituted or unsubstituted dibenzofuran ring, a substituted or unsubstituted carbazole ring, or a substituted or unsubstituted dibenzothiophene ring.

In an exemplary embodiment, the substituted or unsubstituted fused aromatic hydrocarbon ring having 10 to 50 ring carbon atoms in the formula (41-3), (41-4) or (41-5) is a substituted or unsubstituted naphthalene ring, or a substituted or unsubstituted fluorene ring; and
the substituted or unsubstituted fused heterocycle having 8 to 50 ring atoms is a substituted or unsubstituted dibenzofuran ring, a substituted or unsubstituted carbazole ring, or a substituted or unsubstituted dibenzothiophene ring.

In an exemplary embodiment, the compound represented by the formula (4) is selected from the group consisting of a compound represented by a formula (461) below, a compound represented by a formula (462) below, a compound represented by a formula (463) below, a compound represented by a formula (464) below, a compound represented by a formula (465) below, a compound represented by a formula (466) below, and a compound represented by a formula (467) below.

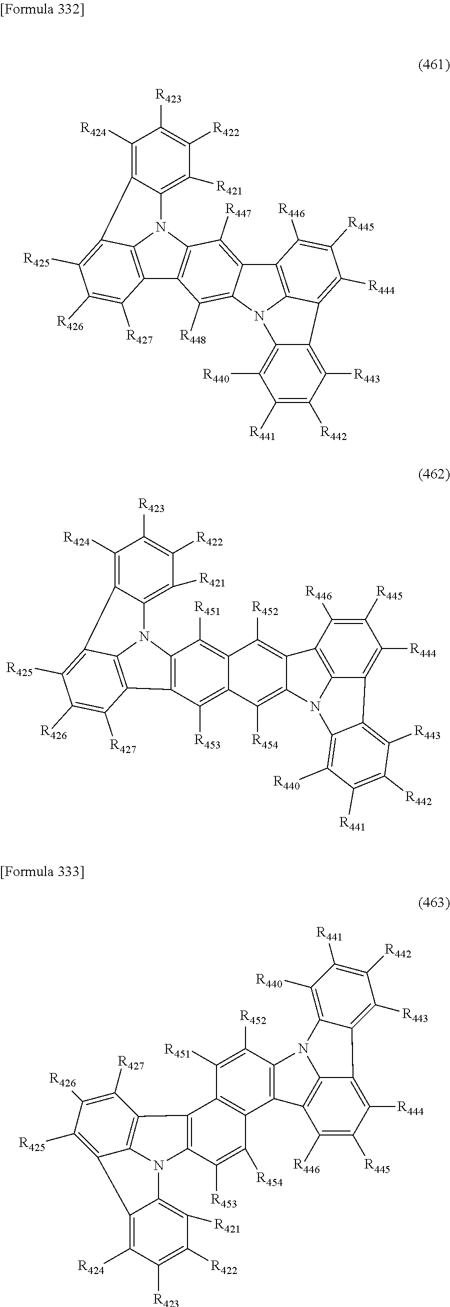

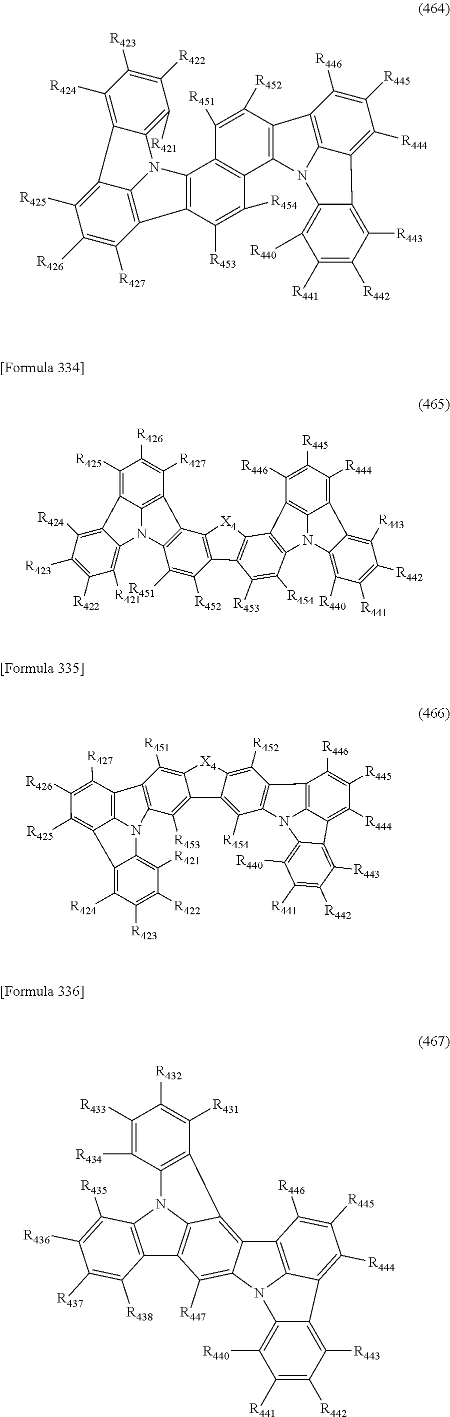

In the formulae (461) to (467): at least one combination of adjacent two or more of moieties selected from R₄₂₁ to R₄₂₇, R₄₃₁ to R₄₃₆, R₄₄₀ to R₄₄₈, and R₄₅₁ to R₄₅₄ are mutually bonded to form a substituted or unsubstituted monocyclic ring, mutually bonded to form a substituted or unsubstituted fused ring, or not mutually bonded;
R₄₃₇, R₄₃₈, and R₄₂₁ to R₄₂₇, R₄₃₁ to R₄₃₆, R₄₄₀ to R₄₄₈, and R₄₅₁ to R₄₅₄ not forming the monocyclic ring and not forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
X₄ is an oxygen atom, NR₈₀₁, or C(R₈₀₂)(R₈₀₃);
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

In an exemplary embodiment, R₄₂₁ to R₄₂₇ and R₄₄₀ to R₄₄₈ are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₄₂₁ to R₄₂₇ and R₄₄₀ to R₄₄₇ are each independently selected from the group consisting of a hydrogen atom, a substituted or unsubstituted aryl group having 6 to 18 ring carbon atoms, and a substituted or unsubstituted heterocyclic group having 5 to 18 ring atoms.

In an exemplary embodiment, the compound represented by the formula (41-3) is represented by a formula (41-3-1) below.

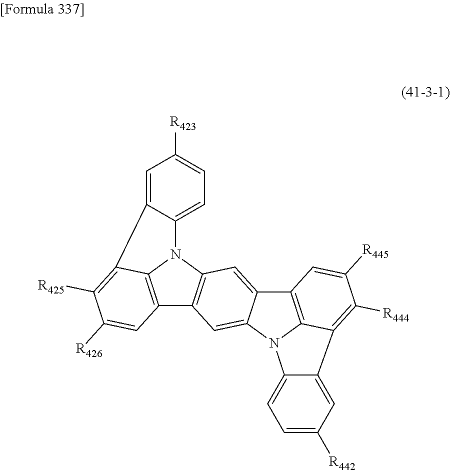

In the formula (41-3-1), R₄₂₃, R₄₂₅, R₄₂₆, R₄₄₂, R₄₄₄ and R₄₄₅ each independently represent the same as R₄₂₃, R₄₂₅, R₄₂₆, R₄₄₂, R₄₄₄ and R₄₄₅ in the formula (41-3).

In an exemplary embodiment, the compound represented by the formula (41-3) is represented by a formula (41-3-2) below.

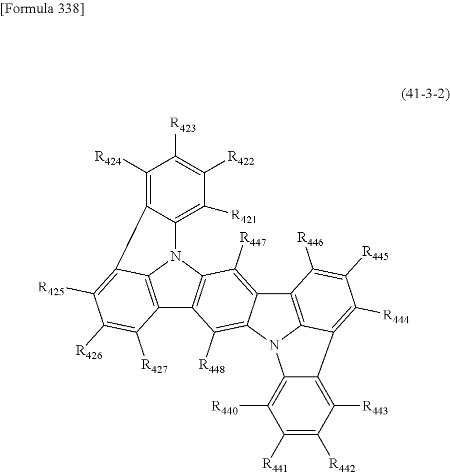

In the formula (41-3-2), R₄₂₁ to R₄₂₇ and R₄₄₀ to R₄₄₈ each independently represent the same as R₄₂₁ to R₄₂₇ and R₄₄₀ to R₄₄₈ in the formula (41-3); and
at least one of R₄₂₁ to R₄₂₇ or R₄₄₀ to R₄₄₆ is a group represented by —N(R₉₀₆)(R₉₀₇).

In an exemplary embodiment, two of R₄₂₁ to R₄₂₇ and R₄₄₀ to R₄₄₆ in the formula (41-3-2) are groups represented by —N(R₉₀₆)(R₉₀₇).

In an exemplary embodiment, the compound represented by the formula (41-3-2) is represented by a formula (41-3-3) below.

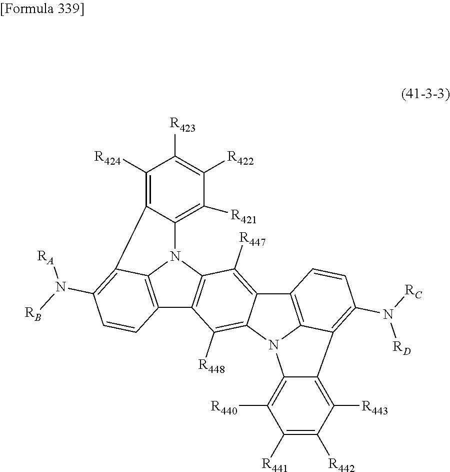

In the formula (41-3-3), R₄₂₁ to R₄₂₄, R₄₄₀ to R₄₄₃, R₄₄₇, and R₄₄₈ each independently represent the same as R₄₂₁ to R₄₂₄, R₄₄₀ to R₄₄₃, R₄₄₇, and R₄₄₈ in the formula (41-3); and
R_{A}, R_{B}, R_{C}, and R_{D} are each independently a substituted or unsubstituted aryl group having 6 to 18 ring carbon atoms, or a substituted or unsubstituted heterocyclic group having 5 to 18 ring atoms.

In an exemplary embodiment, the compound represented by the formula (41-3-3) is represented by a formula (41-3-4) below.

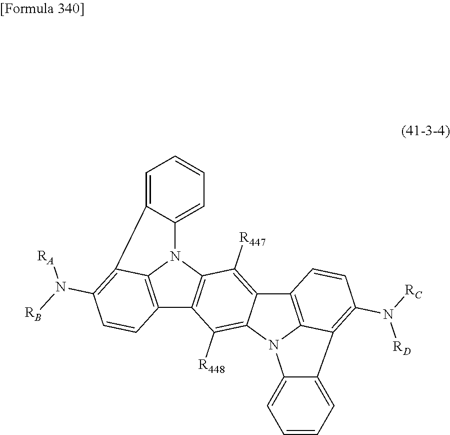

In the formula (41-3-4), R₄₄₇, R₄₄₈, R_{A}, R_{B}, R_{C} and R_{D} each independently represent the same as R₄₄₇, R₄₄₈, RA, RB, R_{C} and R_{D} in the formula (41-3-3).

In an exemplary embodiment, R_{A}, R_{B}, R_{C}, and R_{D} are each independently a substituted or unsubstituted aryl group having 6 to 18 ring carbon atoms.

In an exemplary embodiment, R_{A}, R_{B}, R_{C}, and R_{D} are each independently a substituted or unsubstituted phenyl group.

In an exemplary embodiment, R₄₄₇ and R₄₄₈ are each a hydrogen atom.

In an exemplary embodiment, the substituent for “substituted or unsubstituted” is an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁ₐ)(R₉₀₂ₐ)(R₉₀₃ₐ), —O—(R₉₀₄ₐ), —S—(R₉₀₅ₐ), —N(R₉₀₆ₐ)(R₉₀₇ₐ), a halogen atom, a cyano group, a nitro group, an unsubstituted aryl group having 6 to 50 ring carbon atoms, or an unsubstituted heterocyclic group having 5 to 50 ring atoms;
R₉₀₁ₐ to R₉₀₇ₐ are each independently a hydrogen atom, an unsubstituted alkyl group having 1 to 50 carbon atoms, an unsubstituted aryl group having 6 to 50 ring carbon atoms, or an unsubstituted heterocyclic group having 5 to 50 ring atoms;
when two or more R₉₀₁ₐ are present, the two or more R₉₀₁ₐ are mutually the same or different;
when two or more R₉₀₂ₐ are present, the two or more R₉₀₂ₐ are mutually the same or different;
when two or more R₉₀₃ₐ are present, the two or more R₉₀₃ₐ are mutually the same or different;
when two or more R₉₀₄ₐ are present, the two or more R₉₀₄ₐ are mutually the same or different;
when two or more R₉₀₅ₐ are present, the two or more R₉₀₅ₐ are mutually the same or different;
when two or more R₉₀₆ₐ are present, the two or more R₉₀₆ₐ are mutually the same or different; and
when two or more R₉₀₇ₐ are present, the two or more R₉₀₇ₐ are mutually the same or different.

In an exemplary embodiment, the substituent for “substituted or unsubstituted” is an unsubstituted alkyl group having 1 to 50 carbon atoms, an unsubstituted aryl group having 6 to 50 ring carbon atoms, or an unsubstituted heterocyclic group having 5 to 50 ring atoms.

In an exemplary embodiment, the substituent for “substituted or unsubstituted” is an unsubstituted alkyl group having 1 to 18 carbon atoms, an unsubstituted aryl group having 6 to 18 ring carbon atoms, or an unsubstituted heterocyclic group having 5 to 18 ring atoms.

### Second Exemplary Embodiment

### Electronic Device

An electronic device according to a second exemplary embodiment is installed with any one of the organic EL devices according to the above exemplary embodiments. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light.

### Modification of Embodiment(s)

The scope of the invention is not limited by the above-described exemplary embodiments but includes any modification and improvement as long as such modification and improvement are compatible with the invention.

For instance, the number of emitting layers is not limited to two, and more than two emitting layers may be provided and laminated with each other. When the organic EL device includes more than two emitting layers, it is only necessary that at least two of the emitting layers should satisfy the requirements mentioned in the above exemplary embodiment. For instance, the rest of the emitting layers may be a fluorescent emitting layer or a phosphorescent emitting layer with use of emission caused by electron transfer from the triplet excited state directly to the ground state.

When the organic EL device includes a plurality of emitting layers, these emitting layers may be mutually adjacently provided, or may form a so-called tandem organic EL device, in which a plurality of emitting units are laminated via an intermediate layer.

For instance, a blocking layer may be provided adjacent to at least one of a side of the emitting layer close to the anode or a side of the emitting layer close to the cathode. The blocking layer is preferably provided in contact with the emitting layer to block at least any of holes, electrons, and excitons.

For instance, when the blocking layer is provided in contact with the side of the emitting layer close to the cathode, the blocking layer permits transport of electrons and blocks holes from reaching a layer provided closer to the cathode (e.g., the electron transporting layer) beyond the blocking layer. When the organic EL device includes the electron transporting layer, the blocking layer is preferably interposed between the emitting layer and the electron transporting layer.

When the blocking layer is provided in contact with the side of the emitting layer close to the anode, the blocking layer permits transport of holes and blocks electrons from reaching a layer provided closer to the anode (e.g., the hole transporting layer) beyond the blocking layer. When the organic EL device includes the hole transporting layer, the blocking layer is preferably interposed between the emitting layer and the hole transporting layer.

Alternatively, the blocking layer may be provided adjacent to the emitting layer so that excitation energy does not leak out from the emitting layer toward neighboring layer(s). The blocking layer blocks excitons generated in the emitting layer from being transferred to a layer(s) (e.g., the electron transporting layer and the hole transporting layer) closer to the electrode(s) beyond the blocking layer.

The emitting layer is preferably bonded with the blocking layer.

Specific structure, shape and the like of the components in the invention may be designed in any manner as long as an object of the invention can be achieved.

### EXAMPLES

The invention will be described in further detail with reference to Examples. It should be noted that the scope of the invention is by no means limited by Examples.

### Compounds

Structures of compounds used as the first host material or the second host material for manufacturing organic EL devices in Examples 1 to 174, A2 to A4, and Reference Example 1 are shown below.

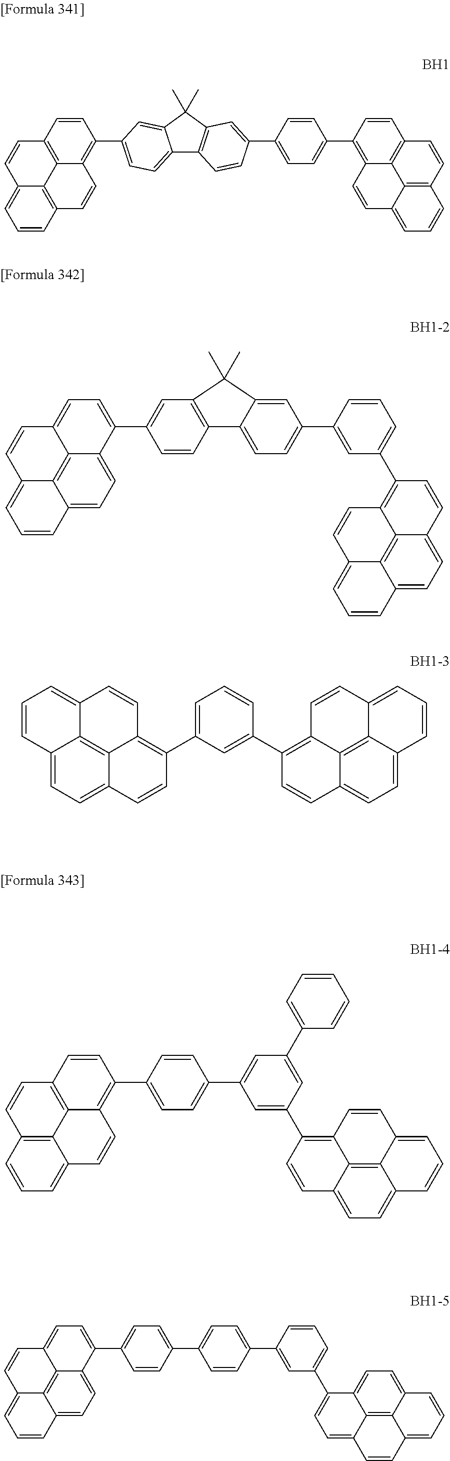

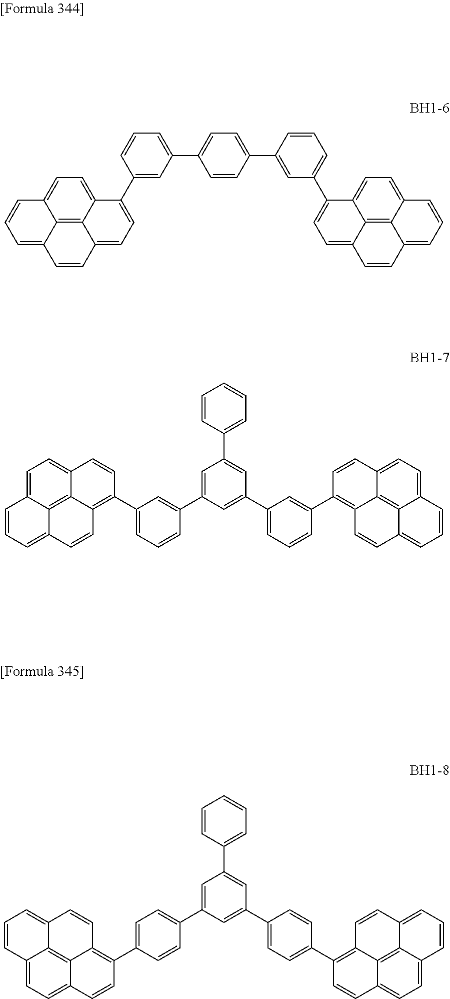

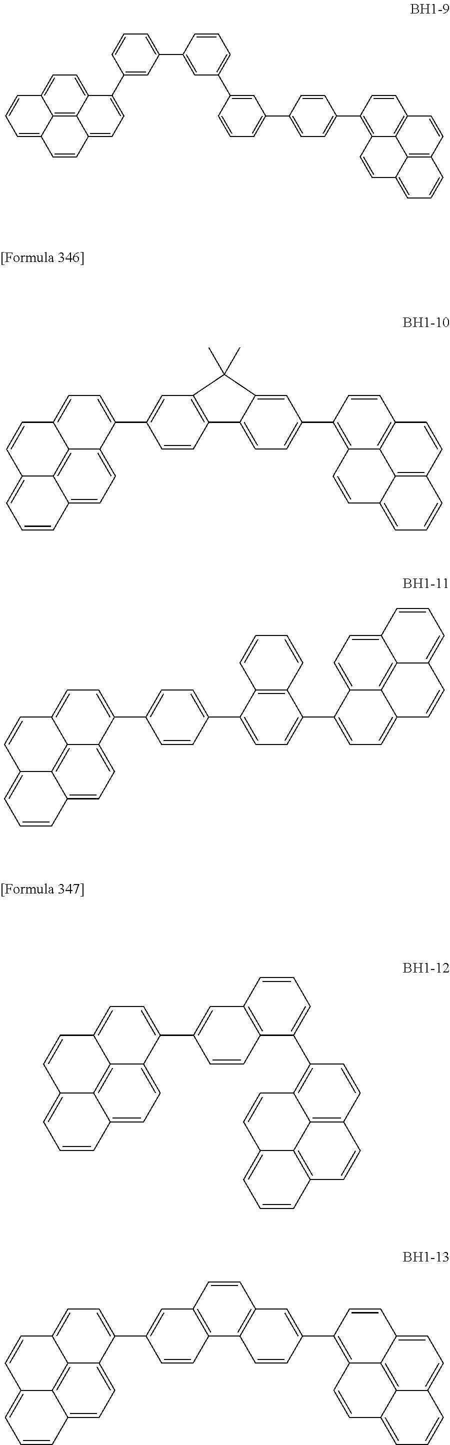

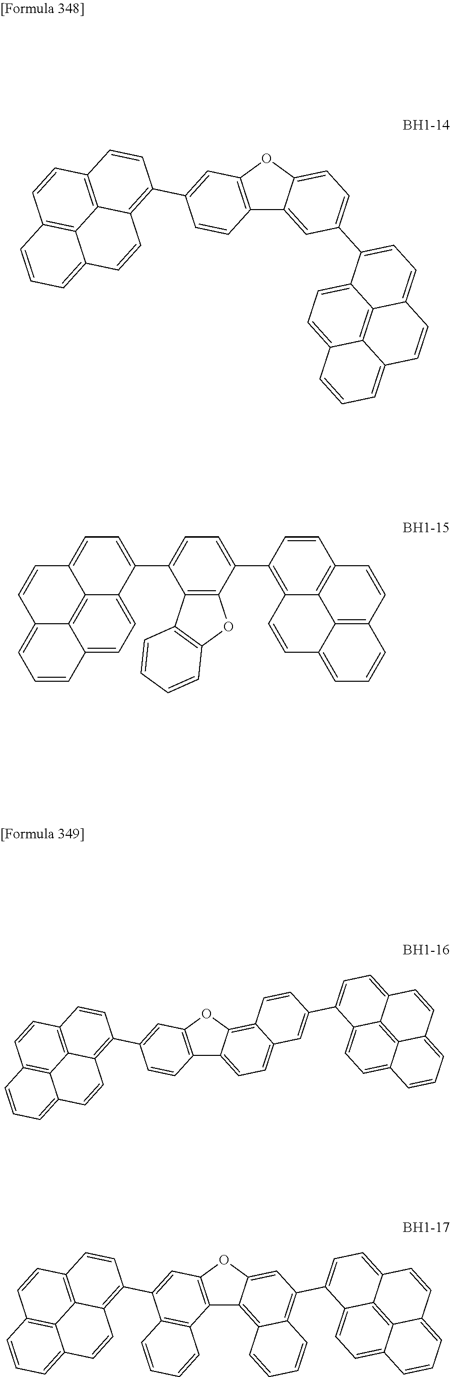 [SMILES: c1cc2ccc3ccc(-c4ccc5c(c4)oc4ccc(-c6ccc7ccc8cccc9ccc6c7c89)cc45)c4ccc(c1)c2c34 ; c1cc2ccc3ccc(-c4ccc(-c5ccc6ccc7cccc8ccc5c6c78)c5c4oc4ccccc45)c4ccc(c1)c2c34 ; c1cc2ccc3ccc(-c4ccc5c(ccc6c7ccc(-c8ccc9ccc%10cccc%11ccc8c9c%10%11)cc7oc56)c4)c4ccc(c1)c2c34 ; c1cc2ccc3ccc(-c4cc5oc6cc(-c7ccc8ccc9cccc%10ccc7c8c9%10)c7ccccc7c6c5c5ccccc45)c4ccc(c1)c2c34]

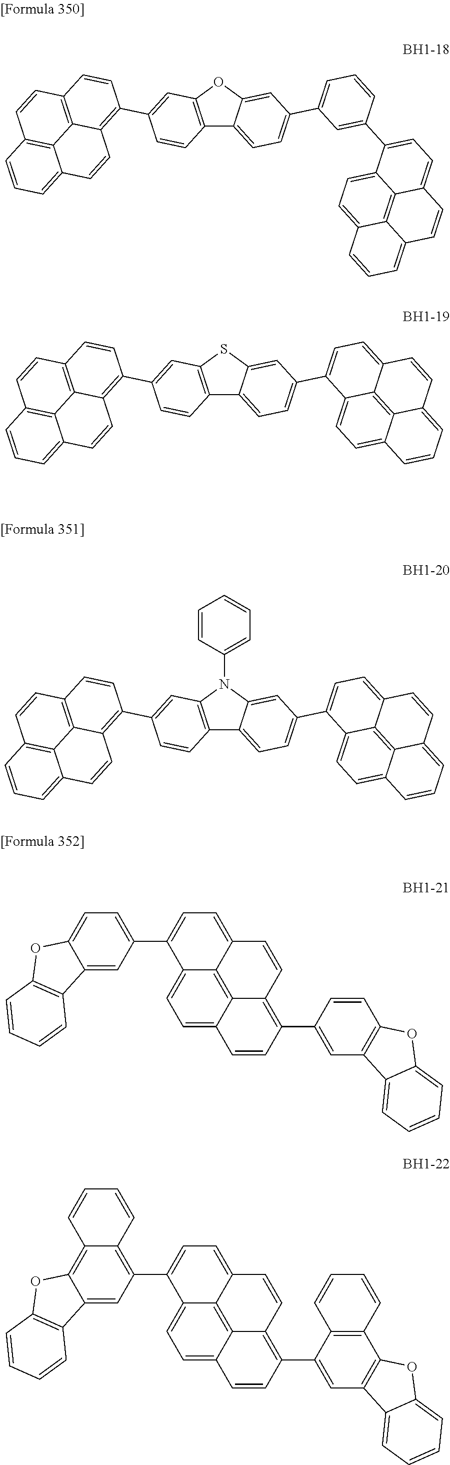 [SMILES: c1cc(-c2ccc3c(c2)oc2cc(-c4ccc5ccc6cccc7ccc4c5c67)ccc23)cc(-c2ccc3ccc4cccc5ccc2c3c45)c1 ; c1cc2ccc3ccc(-c4ccc5c(c4)sc4cc(-c6ccc7ccc8cccc9ccc6c7c89)ccc45)c4ccc(c1)c2c34 ; c1ccc(-n2c3cc(-c4ccc5ccc6cccc7ccc4c5c67)ccc3c3ccc(-c4ccc5ccc6cccc7ccc4c5c67)cc32)cc1 ; c1ccc2c(c1)oc1ccc(-c3ccc4ccc5c(-c6ccc7oc8ccccc8c7c6)ccc6ccc3c4c65)cc12 ; c1ccc2c(c1)oc1c3ccccc3c(-c3ccc4ccc5c(-c6cc7c8ccccc8oc7c7ccccc67)ccc6ccc3c4c65)cc21]

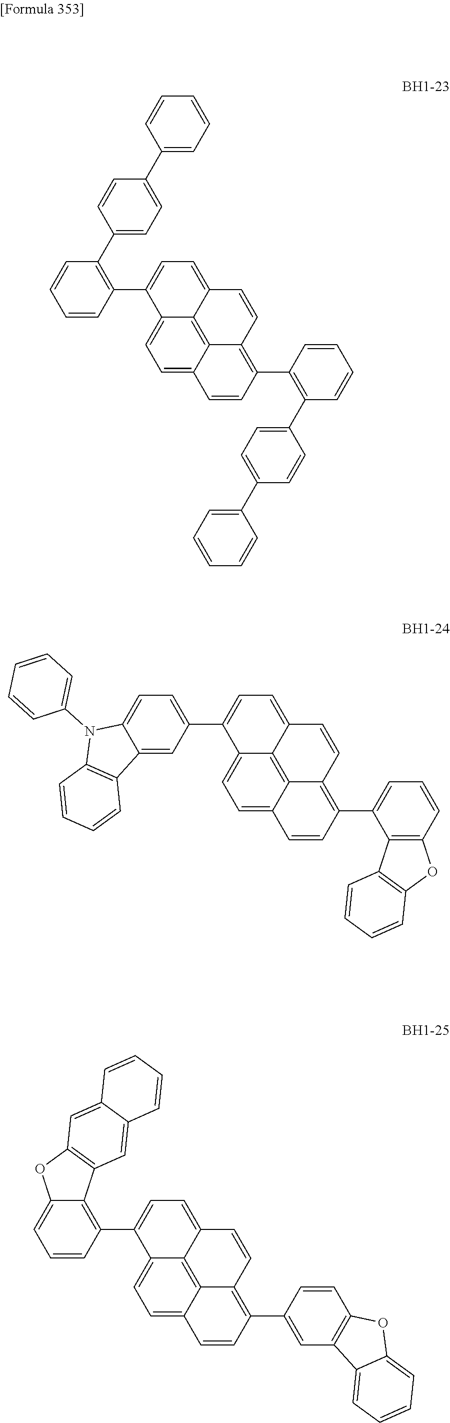

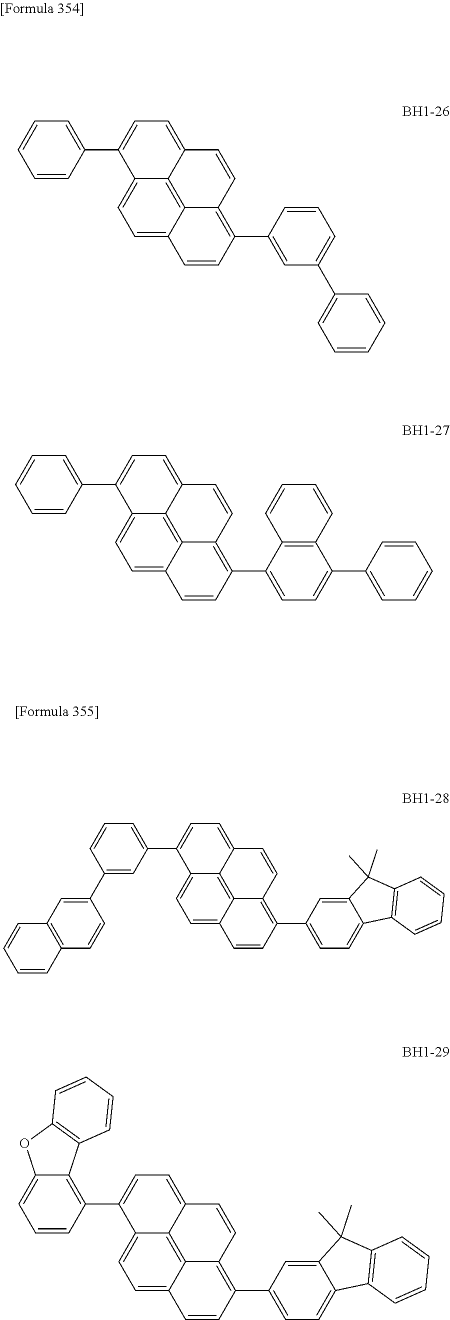

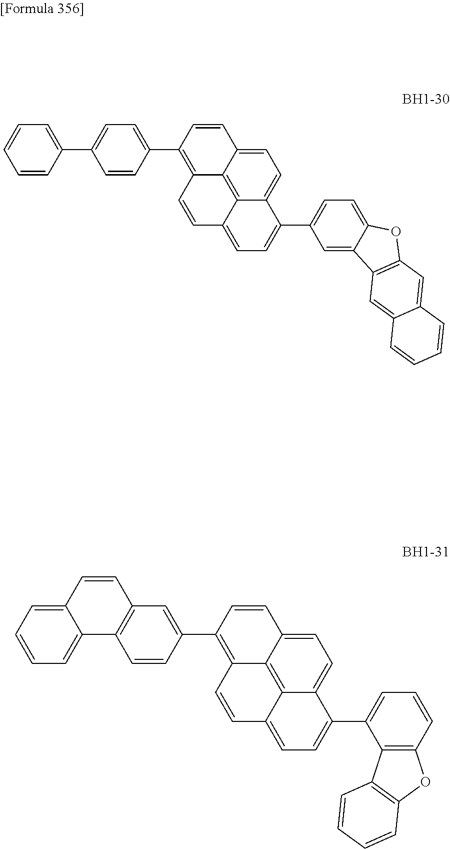 [SMILES: c1ccc(-c2ccc(-c3ccc4ccc5c(-c6ccc7oc8cc9ccccc9cc8c7c6)ccc6ccc3c4c65)cc2)cc1 ; c1ccc2c(c1)ccc1cc(-c3ccc4ccc5c(-c6cccc7oc8ccccc8c67)ccc6ccc3c4c65)ccc12]

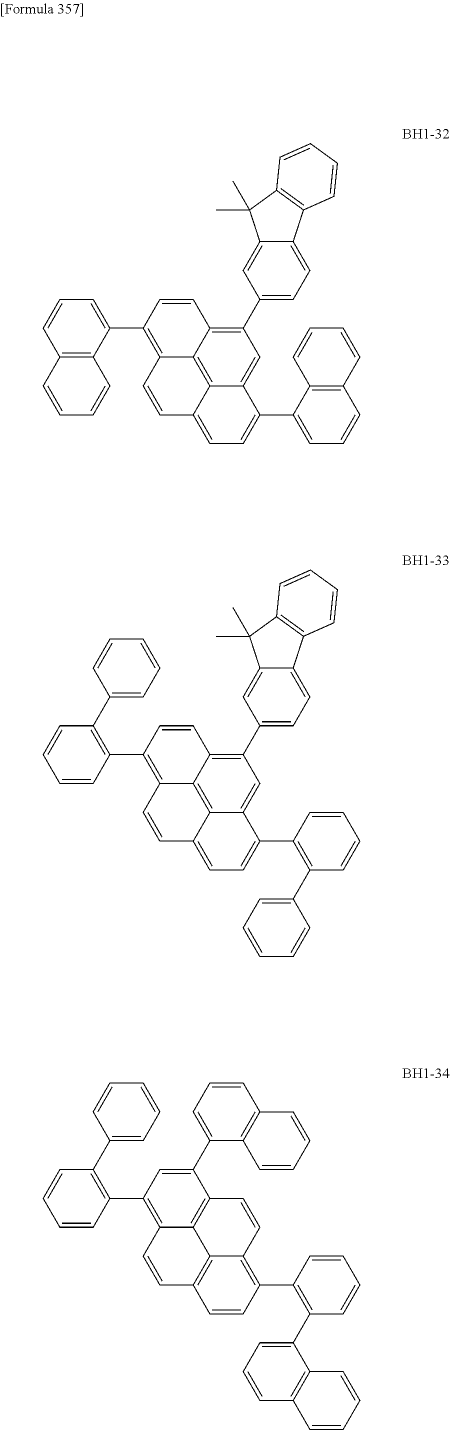

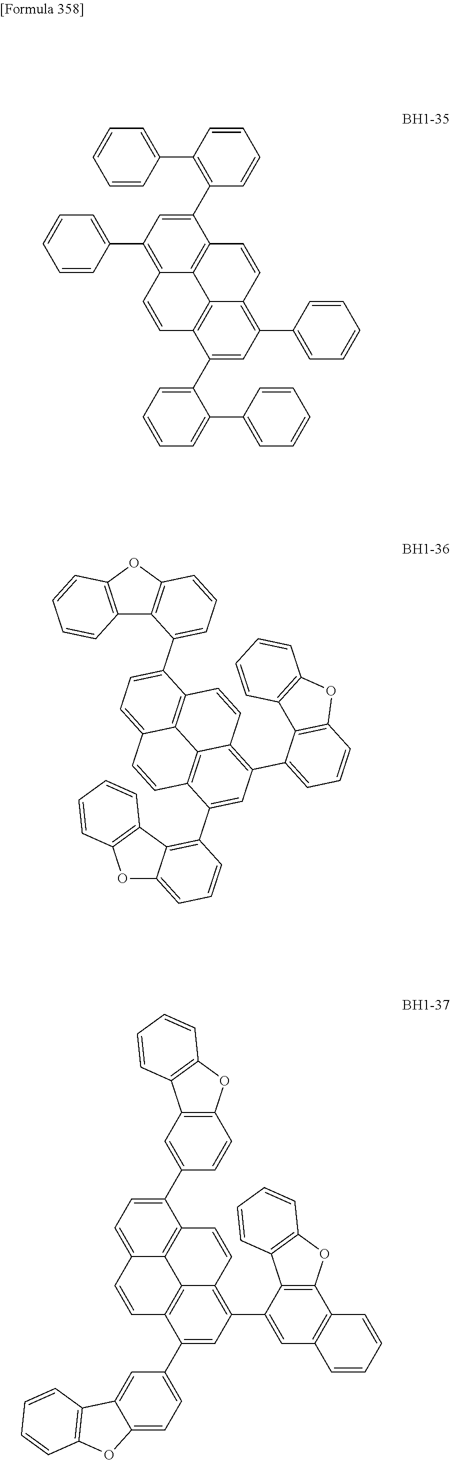

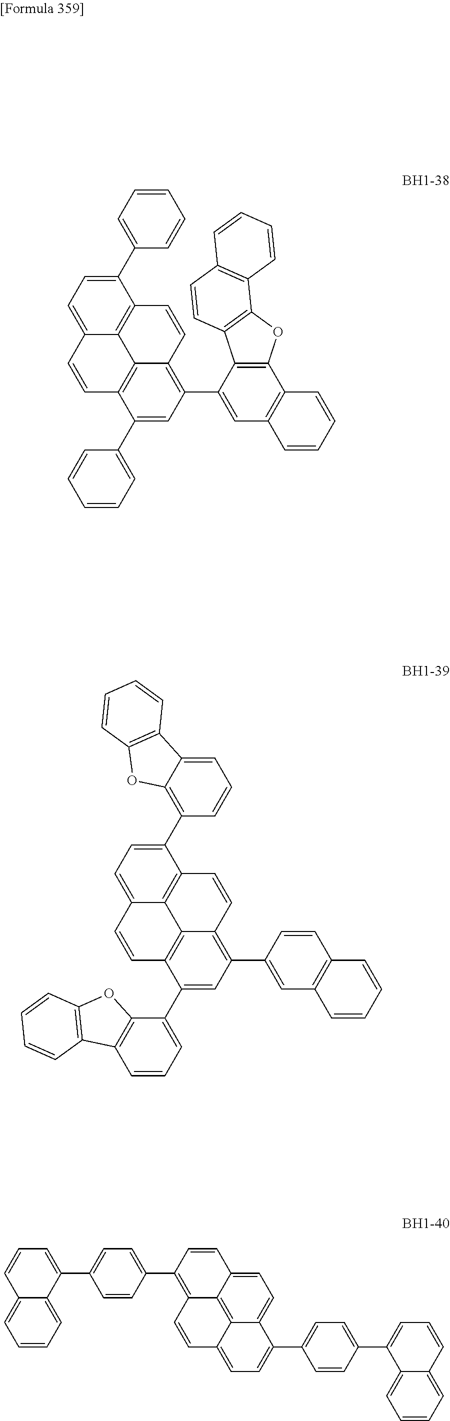

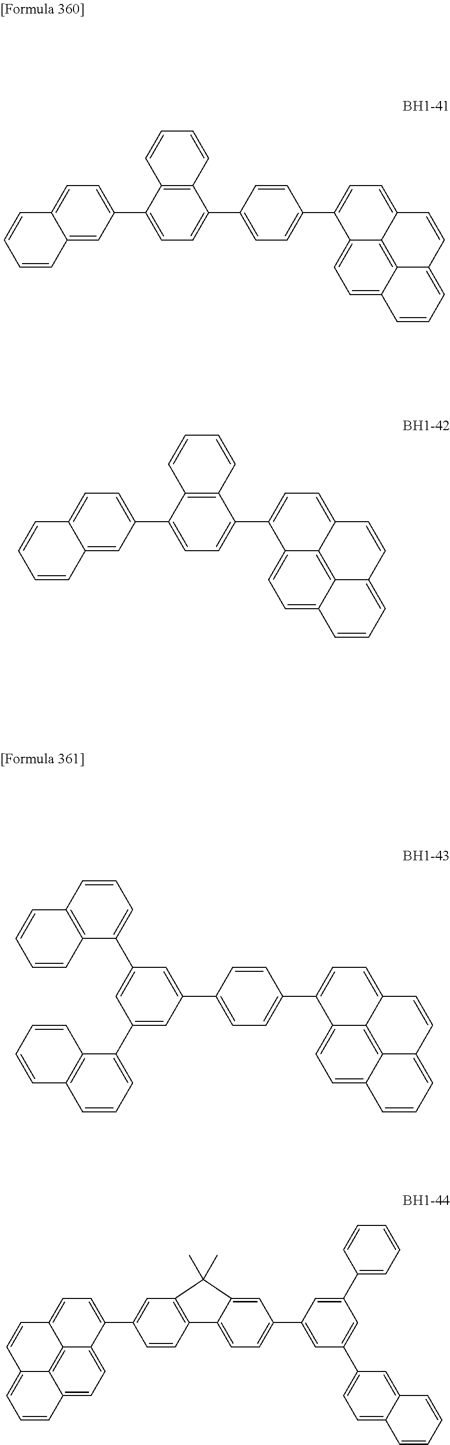

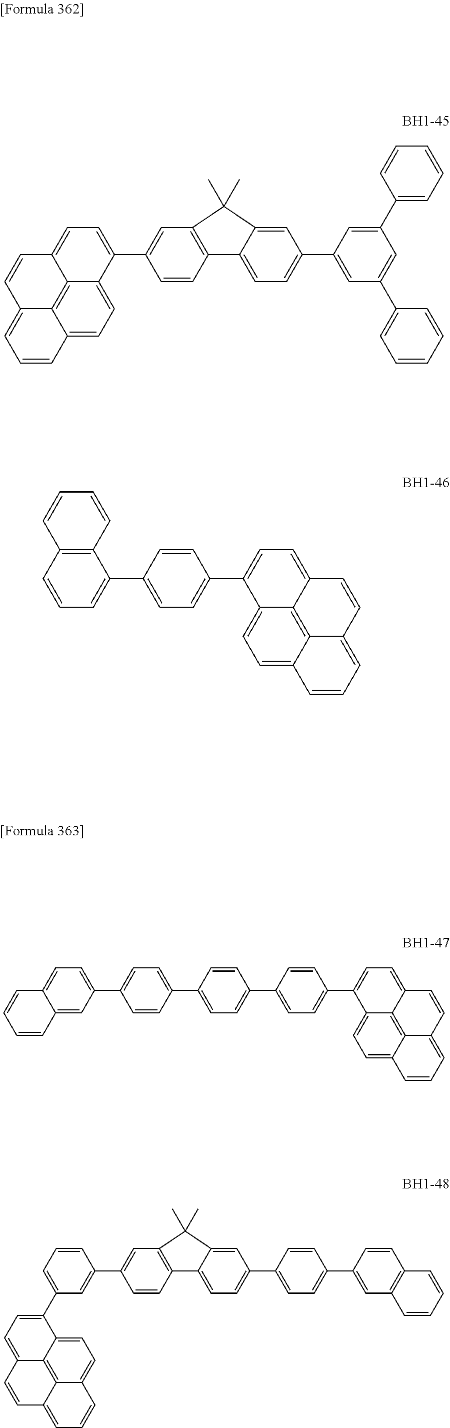

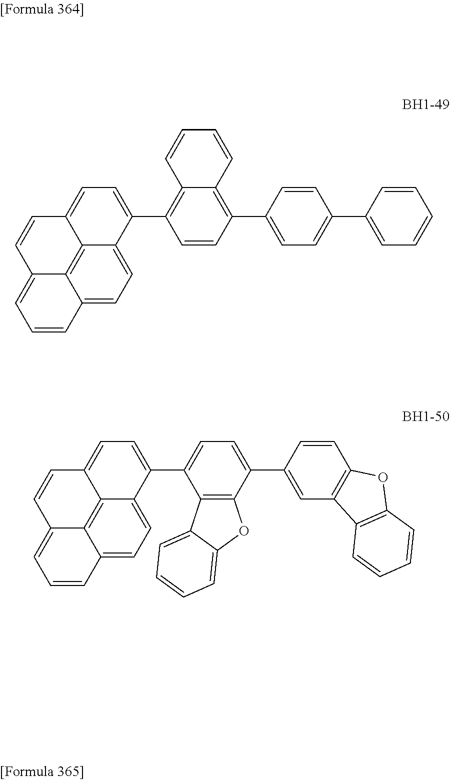

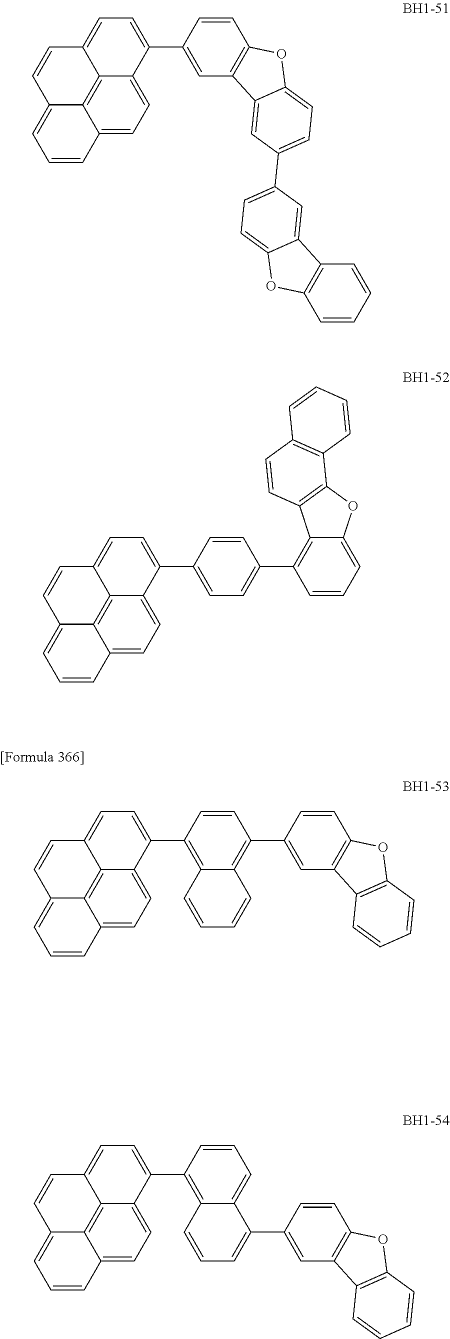 [SMILES: c1cc2ccc3ccc(-c4ccc5oc6ccc(-c7ccc8oc9ccccc9c8c7)cc6c5c4)c4ccc(c1)c2c34 ; c1ccc2c(c1)ccc1c2oc2cccc(-c3ccc(-c4ccc5ccc6cccc7ccc4c5c67)cc3)c21 ; c1cc2ccc3ccc(-c4ccc(-c5ccc6oc7ccccc7c6c5)c5ccccc45)c4ccc(c1)c2c34 ; c1cc2ccc3ccc(-c4cccc5c(-c6ccc7oc8ccccc8c7c6)cccc45)c4ccc(c1)c2c34]

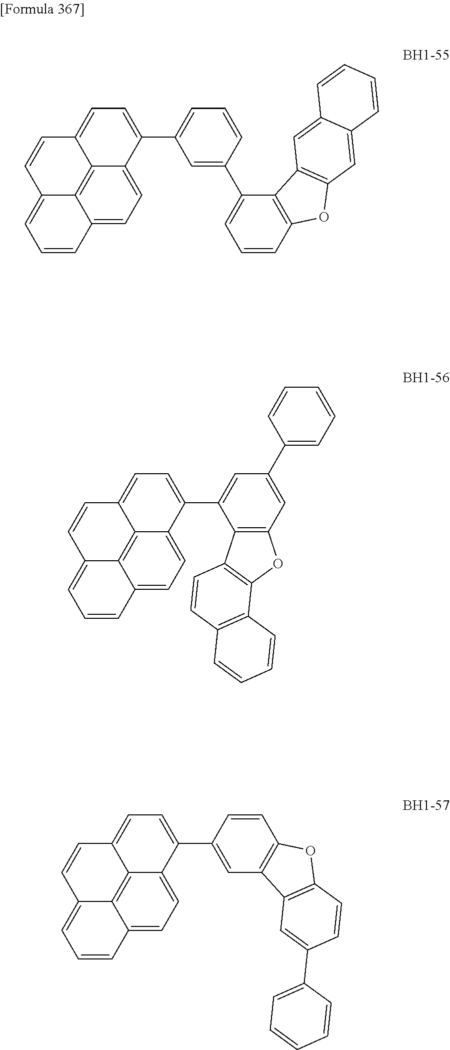 [SMILES: c1cc(-c2ccc3ccc4cccc5ccc2c3c45)cc(-c2cccc3oc4cc5ccccc5cc4c23)c1 ; c1ccc(-c2cc(-c3ccc4ccc5cccc6ccc3c4c56)c3c(c2)oc2c4ccccc4ccc23)cc1 ; c1ccc(-c2ccc3oc4ccc(-c5ccc6ccc7cccc8ccc5c6c78)cc4c3c2)cc1]

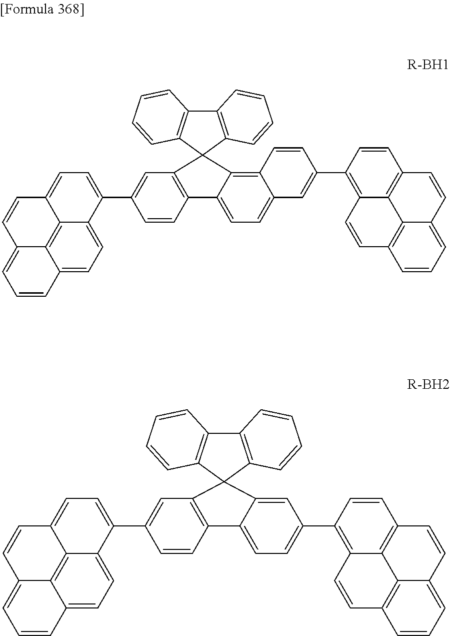

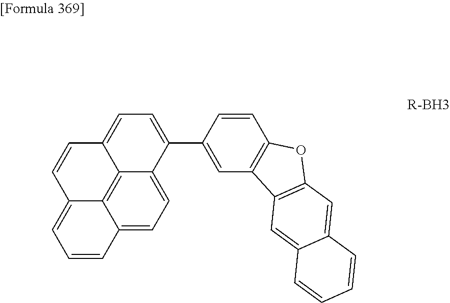 [SMILES: c1ccc2cc3c(cc2c1)oc1ccc(-c2ccc4ccc5cccc6ccc2c4c56)cc13]

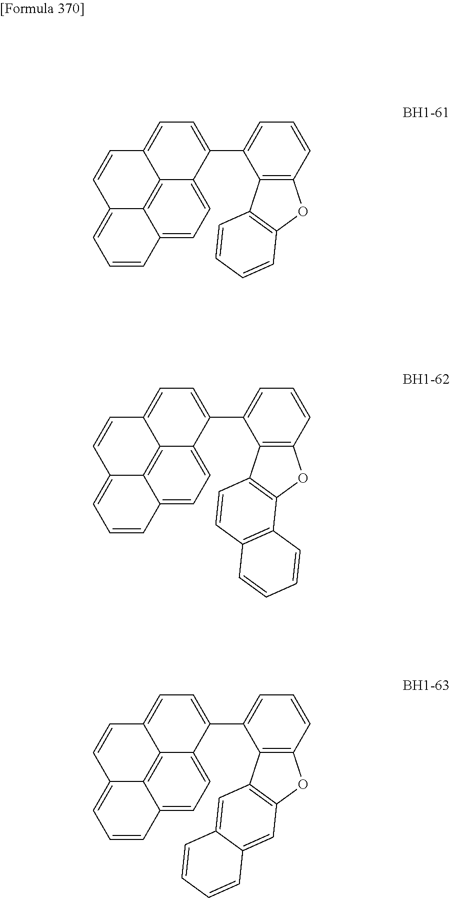 [SMILES: c1cc2ccc3ccc(-c4cccc5oc6ccccc6c45)c4ccc(c1)c2c34 ; c1ccc2c(c1)ccc1c2oc2cccc(-c3ccc4ccc5cccc6ccc3c4c56)c21 ; c1ccc2cc3c(cc2c1)oc1cccc(-c2ccc4ccc5cccc6ccc2c4c56)c13]

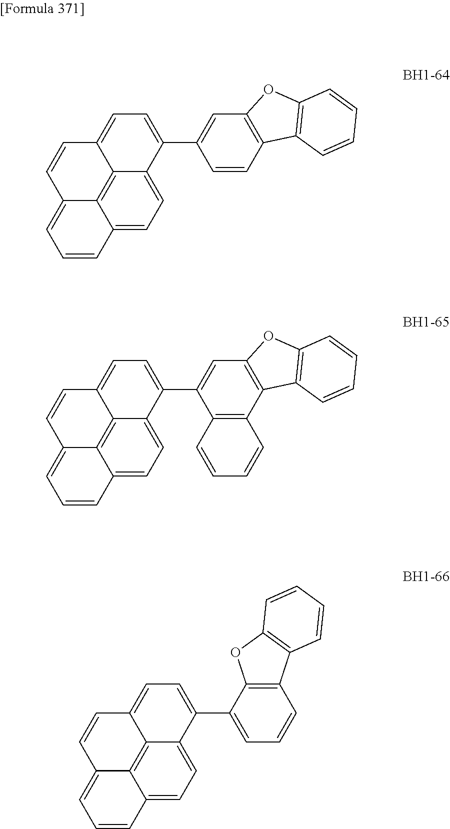 [SMILES: c1cc2ccc3ccc(-c4ccc5c(c4)oc4ccccc45)c4ccc(c1)c2c34 ; c1cc2ccc3ccc(-c4cc5oc6ccccc6c5c5ccccc45)c4ccc(c1)c2c34 ; c1cc2ccc3ccc(-c4cccc5c4oc4ccccc45)c4ccc(c1)c2c34]

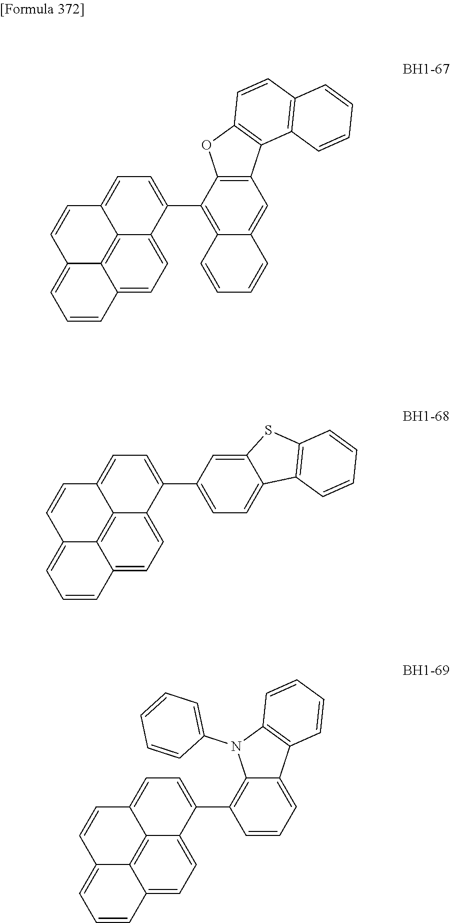 [SMILES: c1ccc2c(-c3ccc4ccc5cccc6ccc3c4c56)c3oc4ccc5ccccc5c4c3cc2c1 ; c1cc2ccc3ccc(-c4ccc5c(c4)sc4ccccc45)c4ccc(c1)c2c34 ; c1ccc(-n2c3ccccc3c3cccc(-c4ccc5ccc6cccc7ccc4c5c67)c32)cc1]

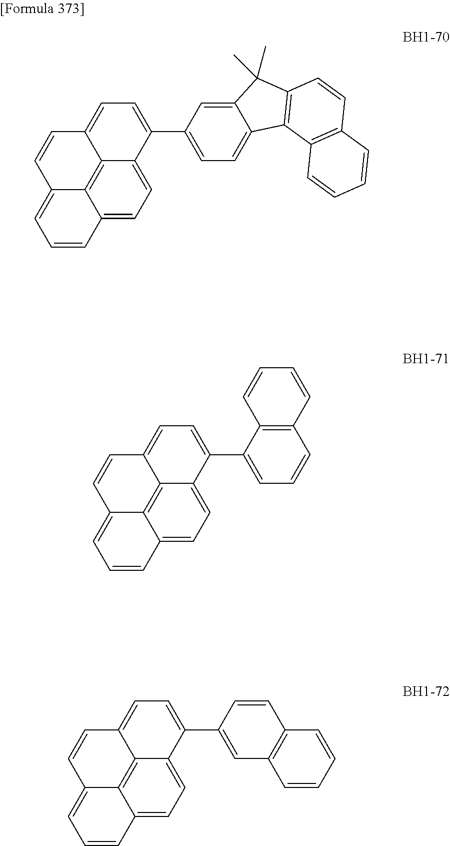

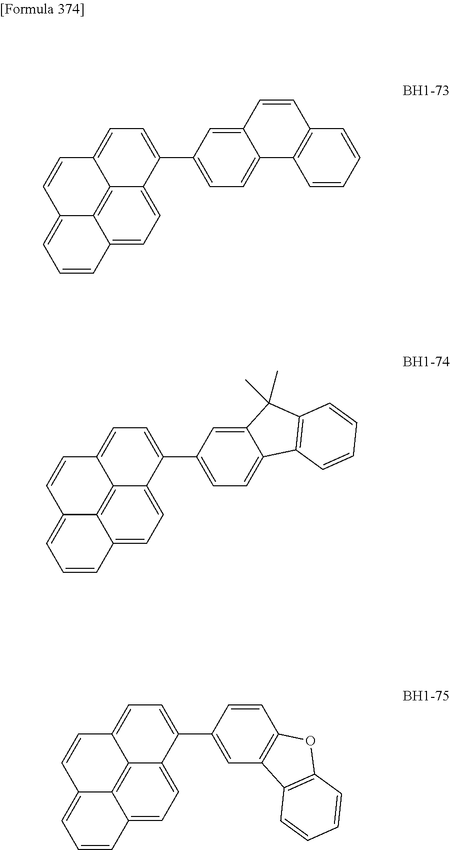

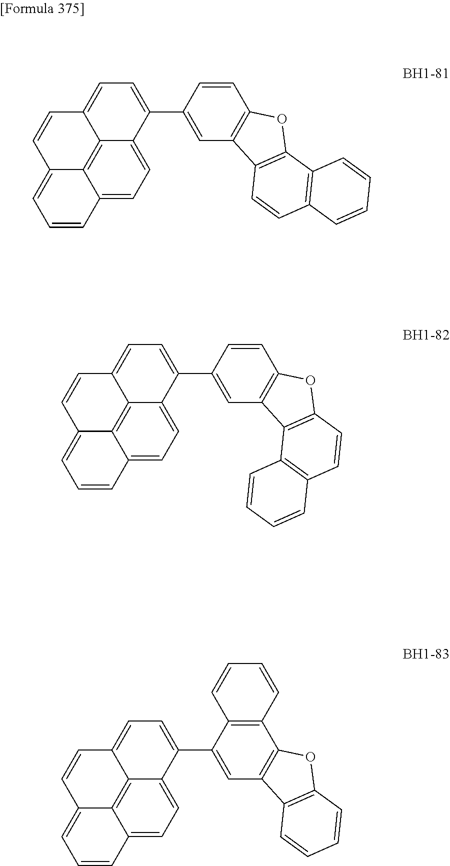 [SMILES: c1ccc2c(c1)ccc1c3cc(-c4ccc5ccc6cccc7ccc4c5c67)ccc3oc21 ; c1ccc2c(c1)ccc1oc3ccc(-c4ccc5ccc6cccc7ccc4c5c67)cc3c12 ; c1cc2ccc3ccc(-c4cc5c6ccccc6oc5c5ccccc45)c4ccc(c1)c2c34]

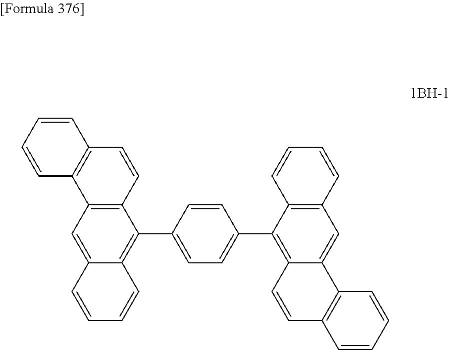 [SMILES: c1ccc2c(-c3ccc(-c4c5ccccc5cc5c4ccc4ccccc45)cc3)c3ccc4ccccc4c3cc2c1]

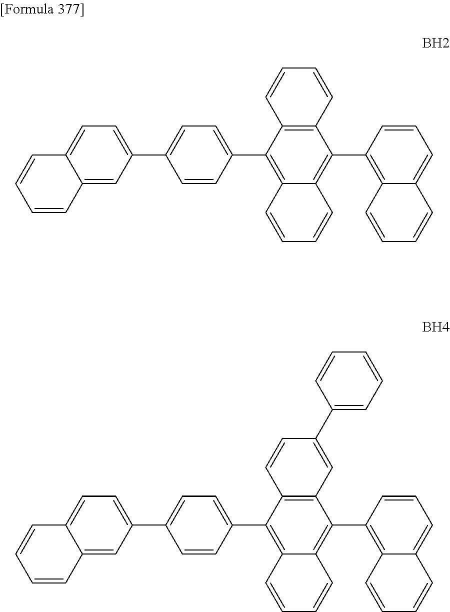

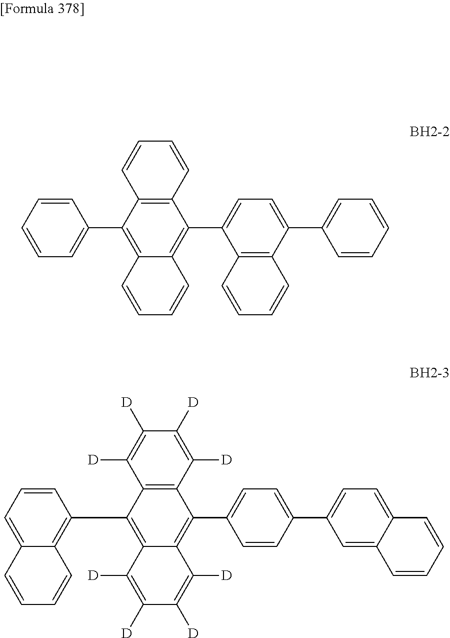

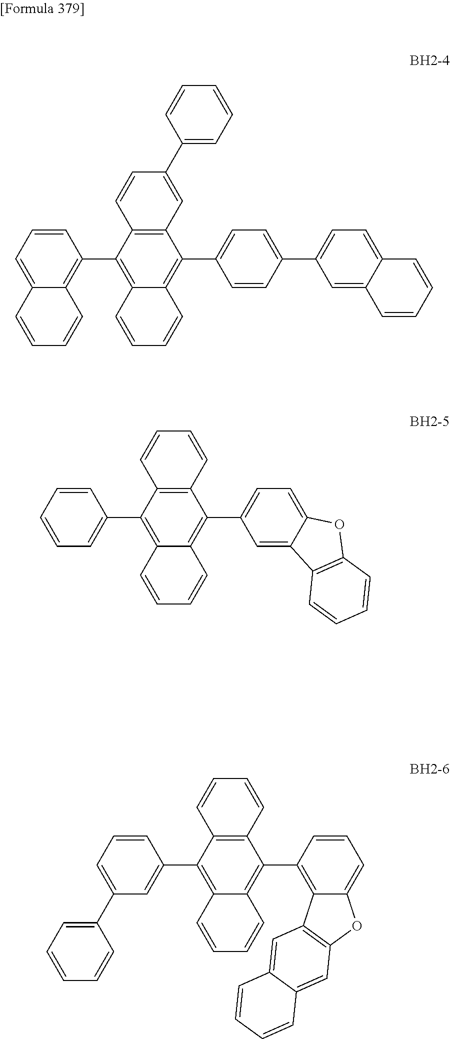

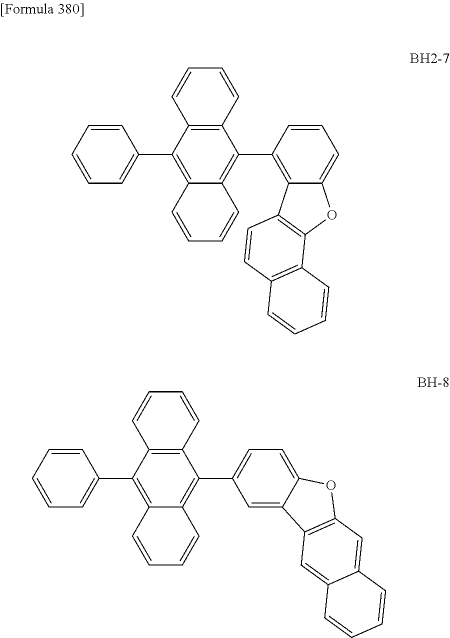 [SMILES: c1ccc(-c2c3ccccc3c(-c3cccc4oc5c6ccccc6ccc5c34)c3ccccc23)cc1 ; c1ccc(-c2c3ccccc3c(-c3ccc4oc5cc6ccccc6cc5c4c3)c3ccccc23)cc1]

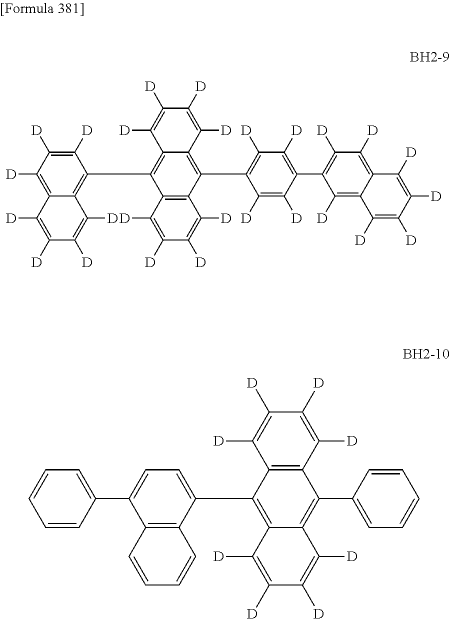 [SMILES: [2H]c1c([2H])c(-c2c3c([2H])c([2H])c([2H])c([2H])c3c(-c3c([2H])c([2H])c([2H])c4c([2H])c([2H])c([2H])c([2H])c34)c3c([2H])c([2H])c([2H])c([2H])c23)c([2H])c([2H])c1-c1c([2H])c([2H])c2c([2H])c([2H])c([2H])c([2H])c2c1[2H] ; [2H]c1c([2H])c([2H])c2c(-c3ccc(-c4ccccc4)c4ccccc34)c3c([2H])c([2H])c([2H])c([2H])c3c(-c3ccccc3)c2c1[2H]]

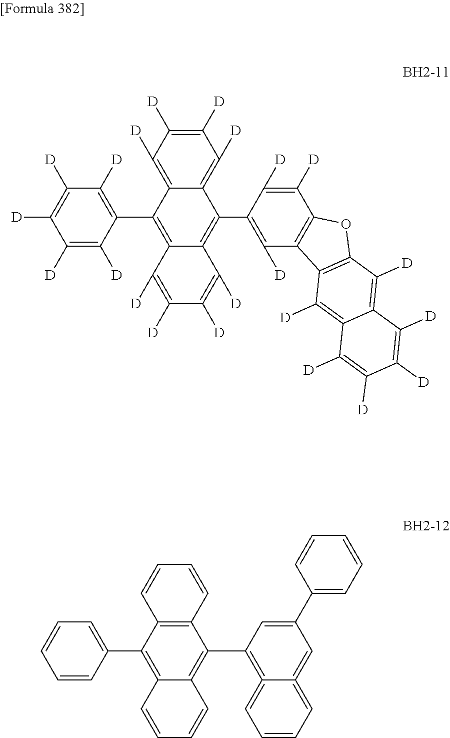

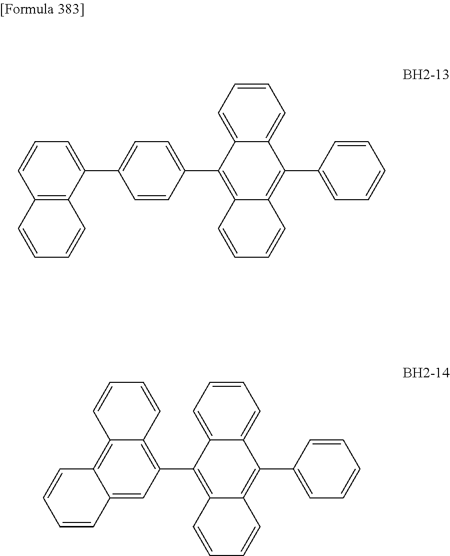

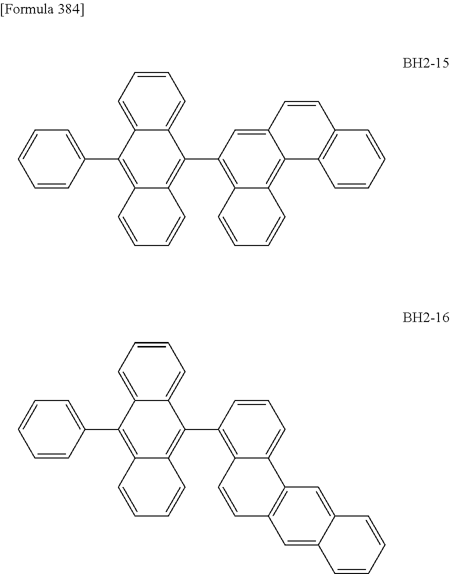

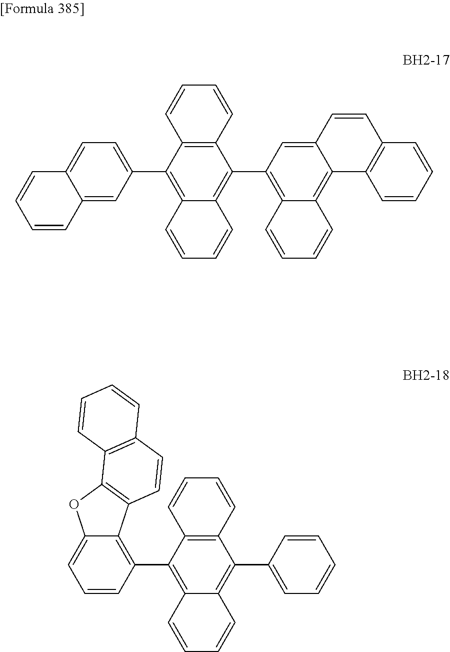

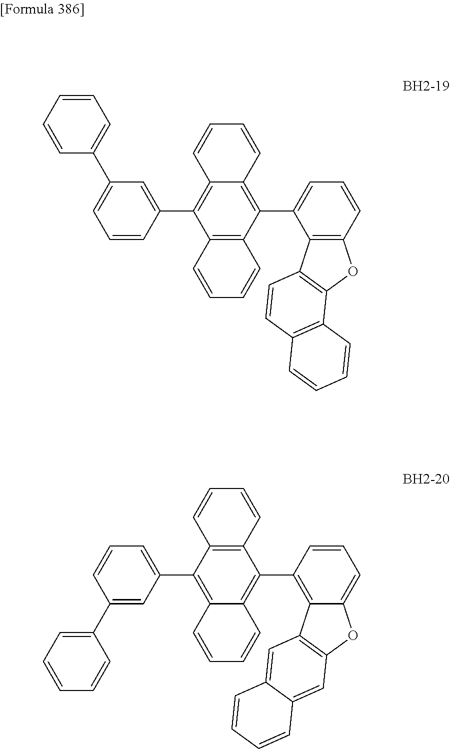 [SMILES: c1ccc(-c2cccc(-c3c4ccccc4c(-c4cccc5oc6c7ccccc7ccc6c45)c4ccccc34)c2)cc1 ; c1ccc(-c2cccc(-c3c4ccccc4c(-c4cccc5oc6cc7ccccc7cc6c45)c4ccccc34)c2)cc1]

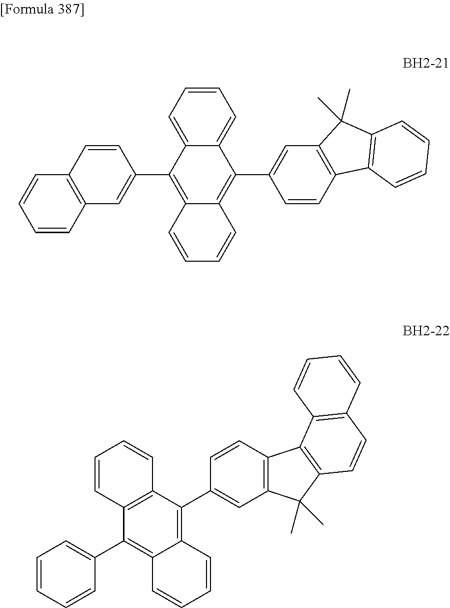

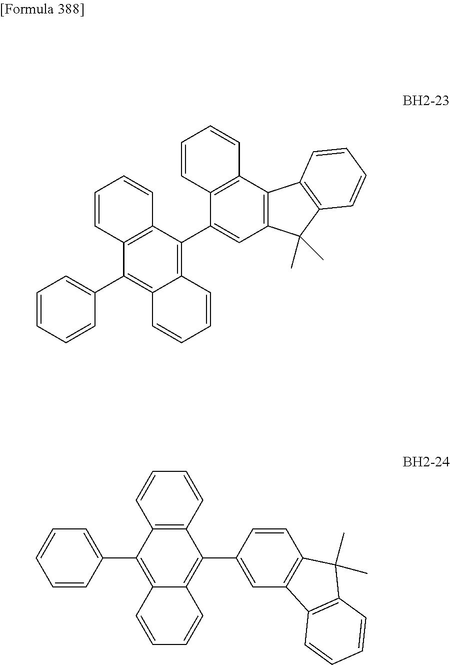

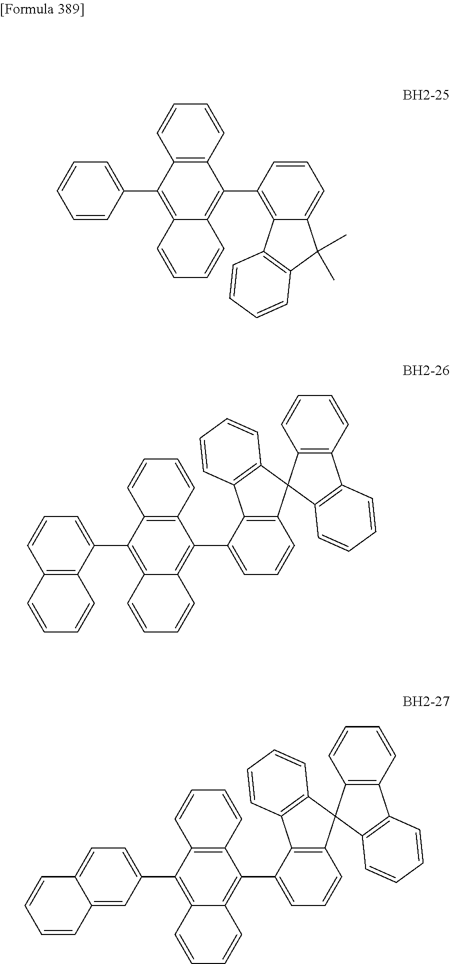

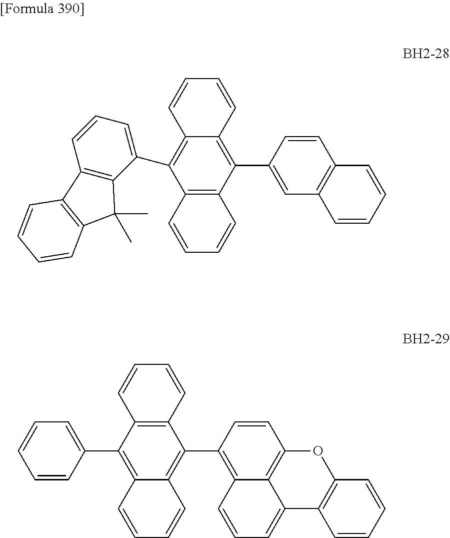

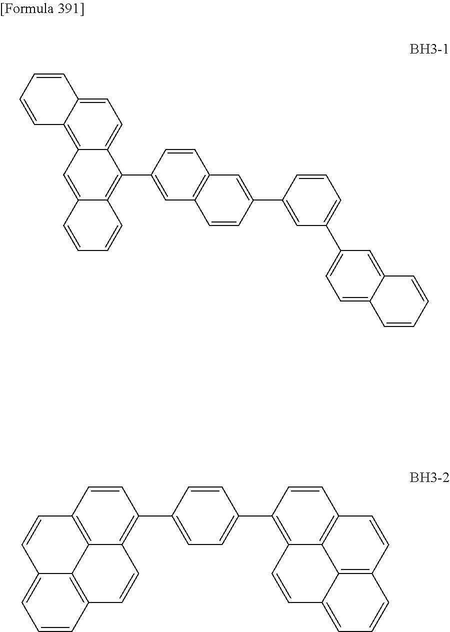

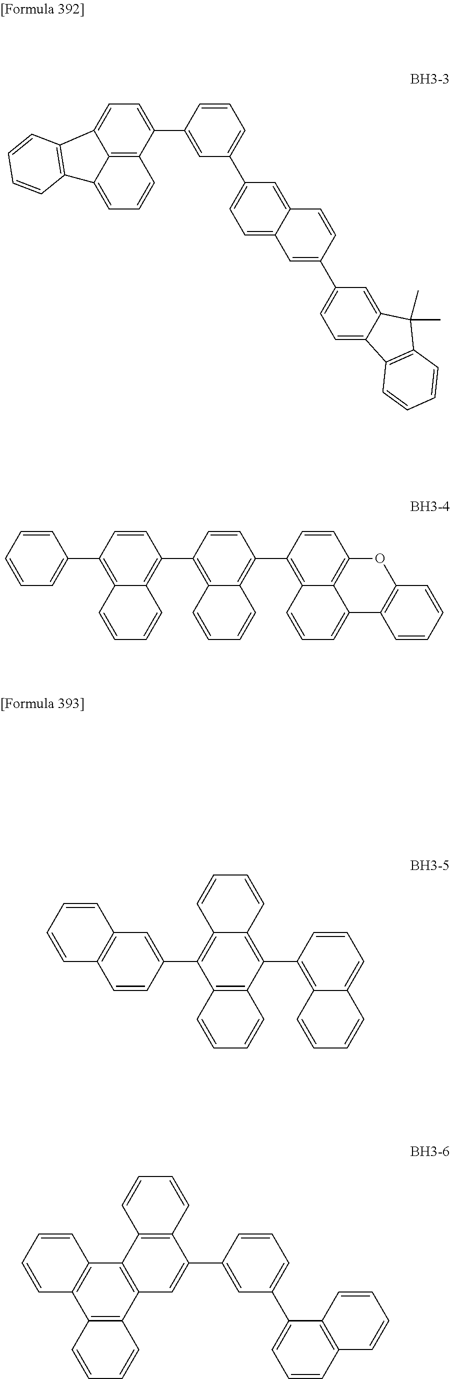

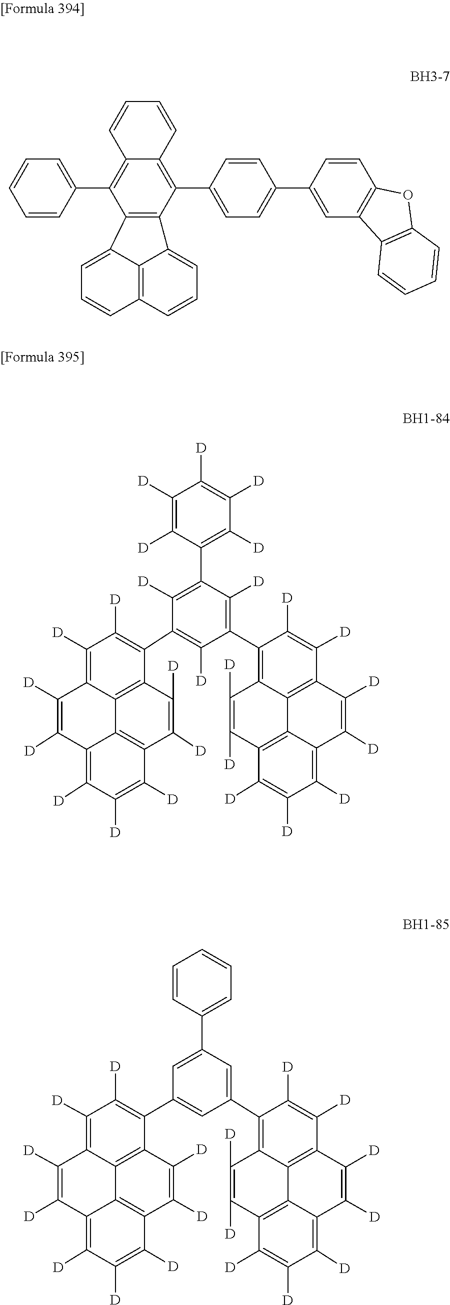 [SMILES: c1ccc(-c2c3c(c(-c4ccc(-c5ccc6oc7ccccc7c6c5)cc4)c4ccccc24)-c2cccc4cccc-3c24)cc1 ; [2H]c1c([2H])c([2H])c(-c2c([2H])c(-c3c([2H])c([2H])c4c([2H])c([2H])c5c([2H])c([2H])c([2H])c6c([2H])c([2H])c3c4c56)c([2H])c(-c3c([2H])c([2H])c4c([2H])c([2H])c5c([2H])c([2H])c([2H])c6c([2H])c([2H])c3c4c56)c2[2H])c([2H])c1[2H] ; [2H]c1c([2H])c2c([2H])c([2H])c3c([2H])c([2H])c(-c4cc(-c5ccccc5)cc(-c5c([2H])c([2H])c6c([2H])c([2H])c7c([2H])c([2H])c([2H])c8c([2H])c([2H])c5c6c78)c4)c4c([2H])c([2H])c(c1[2H])c2c34]

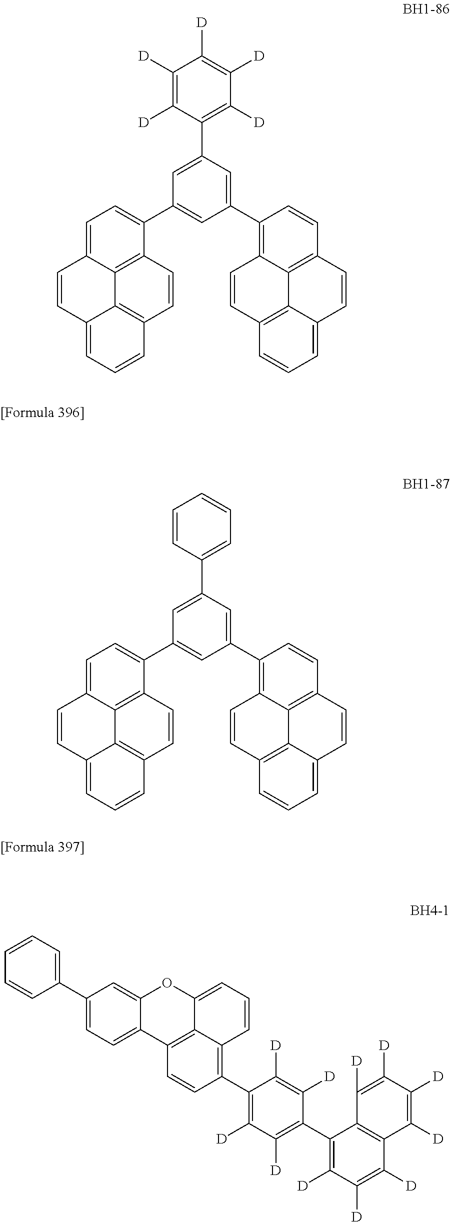

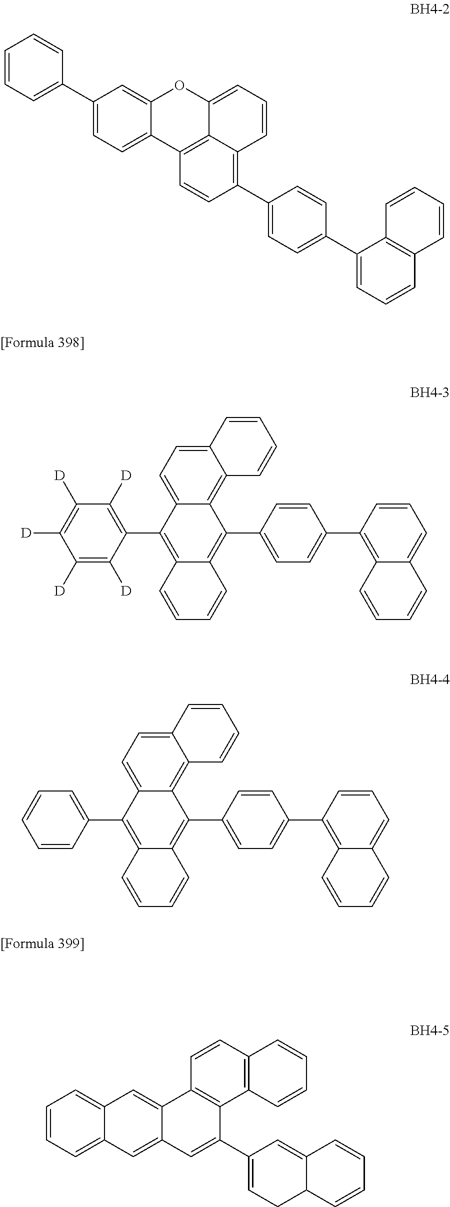

Structures of other compounds used for manufacturing the organic EL devices in Examples 1, A2 to A4, 2 to 174, Reference Example 1, and Comparative Examples 1, 2, A3 to A5, and 3 to 135 are shown below.

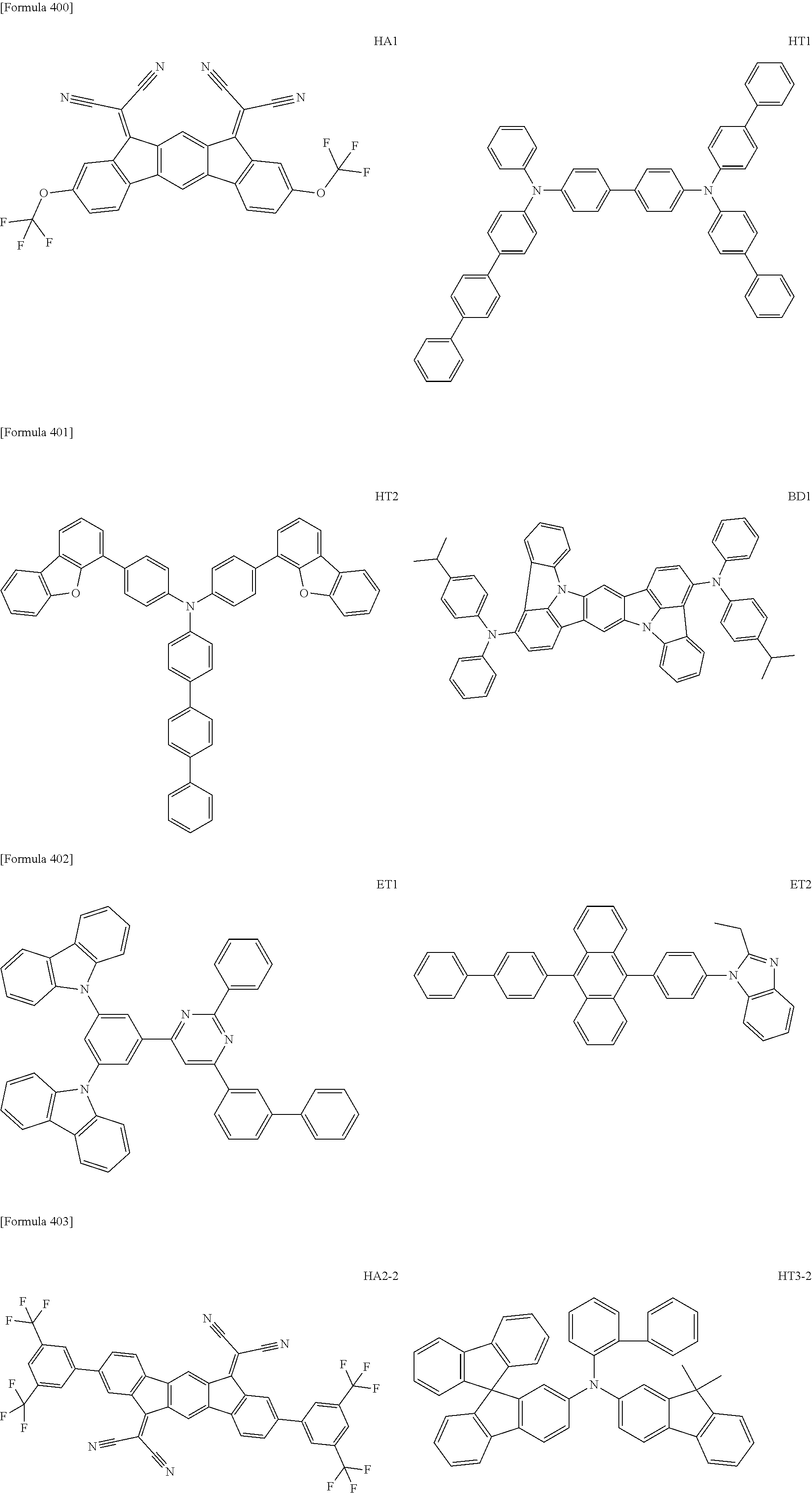

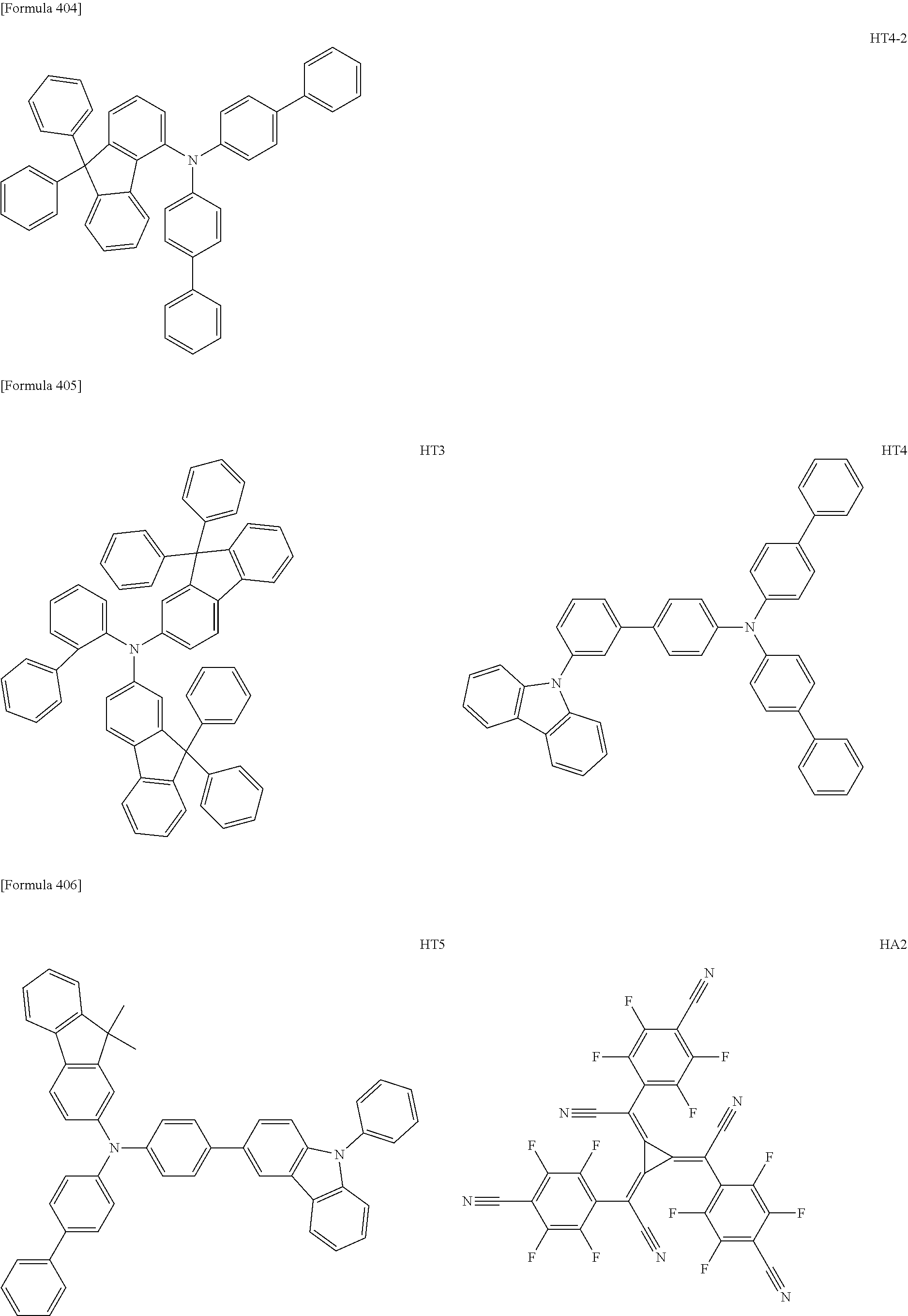 [SMILES: c1ccc(-c2ccc(N(c3ccc(-c4ccccc4)cc3)c3cccc4c3-c3ccccc3C4(c3ccccc3)c3ccccc3)cc2)cc1 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc(-c4ccccc4)cc3)c3ccc(-c4cccc(-n5c6ccccc6c6ccccc65)c4)cc3)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

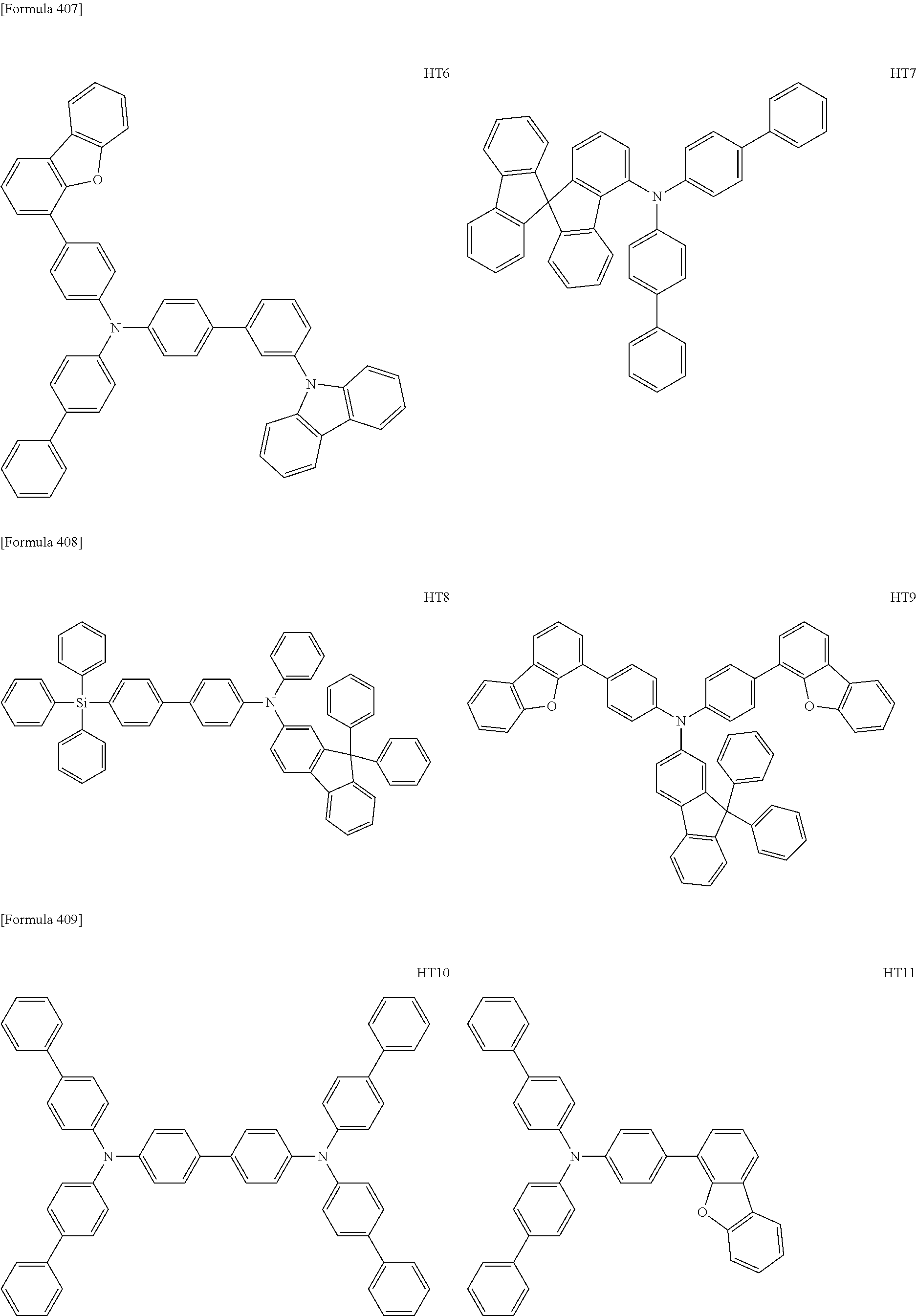

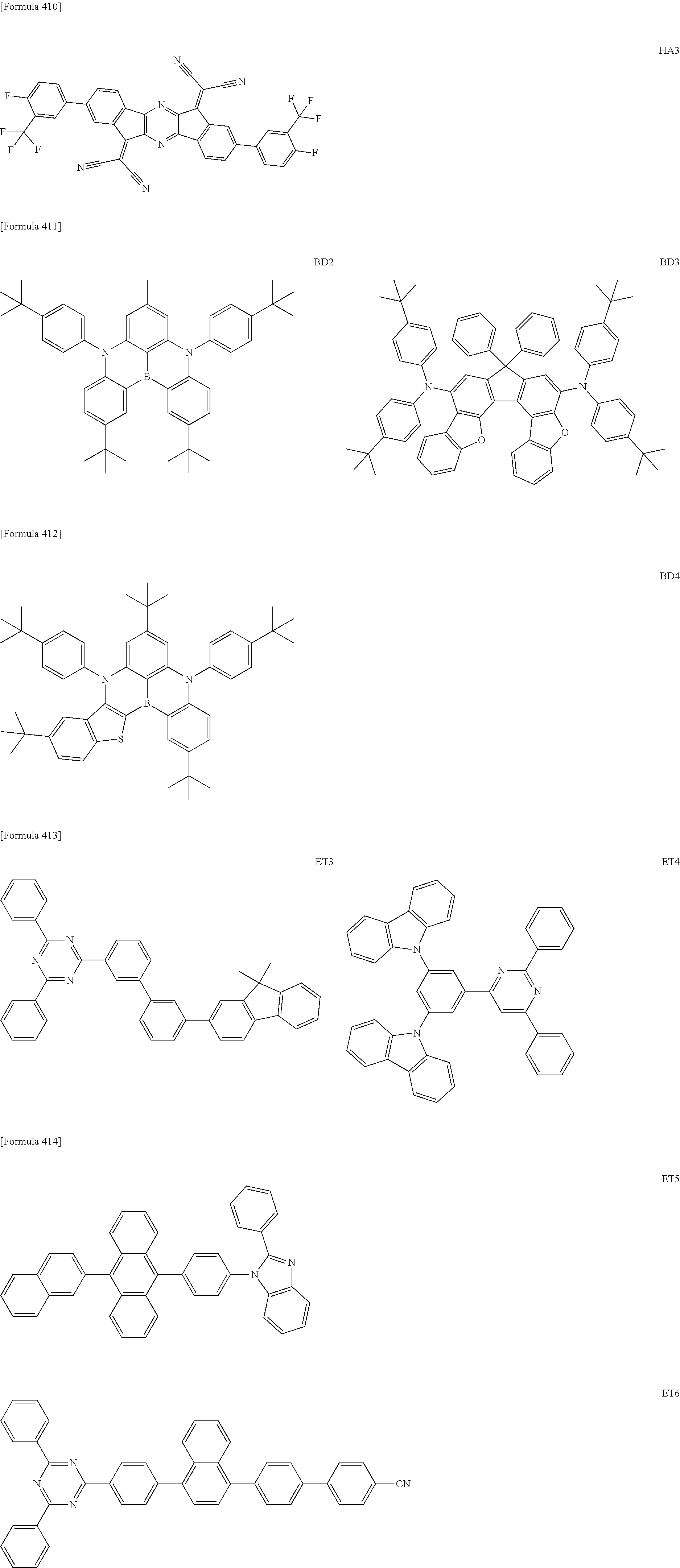

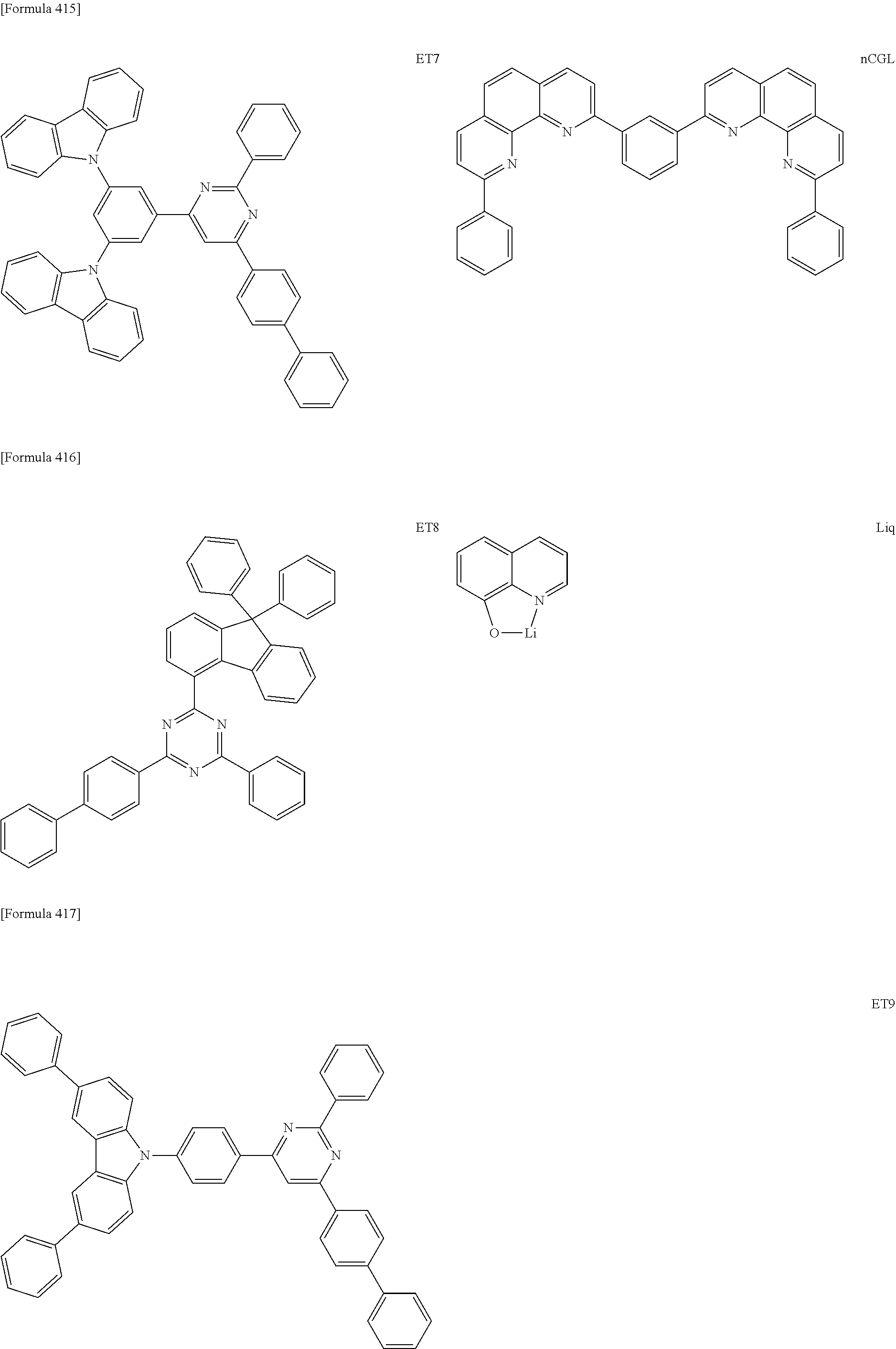

### Manufacture 1 of Organic EL Device

Organic EL devices were manufactured and evaluated as follows.

### Example 1

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. A film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT1 was vapor-deposited to form an 80-nm-thick first hole transporting layer (HT).

After the formation of the first hole transporting layer, the compound HT2 was vapor-deposited to form a 10-nm-thick second hole transporting layer (also referred to as an electron blocking layer (EBL)).

A compound BH1 (first host material (BH)) and a compound BD1 (first dopant material (BD)) were co-deposited on the second hole transporting layer so that a ratio of the compound BD1 accounted for 2 mass %, thereby forming a 5-nm-thick first emitting layer.

A compound BH2 (second host material (BH)) and the compound BD1 (second dopant material (BD)) were co-deposited on the first emitting layer so that a ratio of the compound BD1 accounted for 2 mass %, thereby forming a 20-nm-thick second emitting layer.

The compound ET1 was vapor-deposited on the second emitting layer to form a 10-nm-thick first electron transporting layer (also referred to as a hole blocking layer (HBL)).

The compound ET2 was vapor-deposited on the first electron transporting layer to form a 15-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 1 is roughly shown as follows.

$$ITO ( 130 ) / HA 1 ( 5 ) / HT 1 ( 80 ) / HT 2 ( 10 ) / BH 1 : BD 1 ( 5.98 \% : 2 \% ) / BH 2 : BD 1 ( 20.98 \% : 2 \% ) / ET 1 ( 10 ) / ET 2 ( 15 ) / LiF ( 1 ) / Al ( 80 )$$

The numerals in parentheses represent a film thickness (unit: nm).

The numerals (98%:2%) represented by percentage in the same parentheses indicate a ratio (mass %) between the first host material or the second host material (compound BH1 or compound BH2) and the compound BD1 in each of the first emitting layer and the second emitting layer. Similar notations apply to the description below.

### Comparative Example 1

The organic EL device of Comparative Example 1 was manufactured in the same manner as that of Example 1 except that only the first emitting layer was formed as the emitting layer as shown in Table 1.

### Comparative Example 2

The organic EL device of Comparative Example 2 was manufactured in the same manner as that of Example 1 except that only the second emitting layer was formed as the emitting layer as shown in Table 1.

### Example A2

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, a compound HA2-2 was vapor-deposited on a surface provided with the transparent electrode line to cover the transparent electrode, thereby forming a 5-nm-thick hole injecting layer (HI).

After the formation of the hole injecting layer, a compound HT3-2 was vapor-deposited to form an 80-nm-thick first hole transporting layer (HT).

After the formation of the first hole transporting layer, a compound HT4-2 was vapor-deposited to form a 10-nm-thick second hole transporting layer (also referred to as an electron blocking layer (EBL)).

The compound BH2 (first host material (BH)) and the compound BD1 (first dopant material (BD)) were co-deposited on the second hole transporting layer so that a ratio of the compound BD1 accounted for 2 mass %, thereby forming a 10-nm-thick first emitting layer.

A compound BH4 (second host material (BH)) and the compound BD1 (second dopant material (BD)) were co-deposited on the first emitting layer so that a ratio of the compound BD1 accounted for 2 mass %, thereby forming a 15-nm-thick second emitting layer.

A compound ET7 was deposited on the second emitting layer to form a 10-nm-thick electron transporting layer.

A compound nCGL and metal Li were co-deposited on the electron transporting layer so that a ratio of metal Li accounted for 4 mass %, thereby forming a 30-nm-thick electron injecting layer.

Metal (Al) was vapor-deposited on the electron injecting layer to form a 50-nm-thick cathode.

A device arrangement of the organic EL device in Example A2 is roughly shown as follows.

$$ITO ( 130 ) / HA 2 - 2 ( 5 ) / HT 3 - 2 ( 80 ) / HT 4 - 2 ( 10 ) / BH 2 : BD 1 ( 10 , 98 \% : 2 \% ) / BH 4 : BD 1 ( 15 , 98 \% : 2 \% ) / ET 7 ( 10 ) / nCGL : Li ( 30 , 96 \% : 4 \% ) / Al ( 50 ) )$$

The numerals in parentheses represent a film thickness (unit: nm).

The numerals (98%:2%) represented by percentage in the same parentheses indicate a ratio (mass %) between the first host material or the second host material (compound BH2 or compound BH4) and the compound BD1 in each of the first emitting layer and the second emitting layer. The numerals (96%:4%) represented by percentage in the same parentheses indicate a ratio (mass %) between the compound nCGL ad metal Li in the electron injecting layer. Similar notations apply to the description below.

### Example A3

The organic EL device according to Example A3 was manufactured in the same manner as that of Example A2 except that the compounds and the film thicknesses in the first and second emitting layers of Example A2 were changed to those shown in Table 2.

### Example A4

The organic EL device of Example A4 was manufactured in the same manner as that of Example A3 except that the compounds and the film thicknesses in the first and second emitting layers of Example A3 were changed to those shown in Table 2.

### Comparative Example A3

The organic EL device of Comparative Example A3 was manufactured in the same manner as that of Example A2 except that only the first emitting layer was formed as the emitting layer as shown in Table 2.

### Comparative Example A4

The organic EL device of Comparative Example A4 was manufactured in the same manner as that of Example A2 except that only the second emitting layer was formed as the emitting layer as shown in Table 2.

### Comparative Example A5

The organic EL device of Comparative Example A5 was manufactured in the same manner as that of Example A3 except that only the first emitting layer was formed as the emitting layer as shown in Table 2.

### Evaluation of Organic EL Device

The organic EL devices in Examples 1, A2 to A4, 2 to 174, Reference Example 1, and Comparative Examples 1 to 2, A3 to A5, and 3 to 135 were evaluated as follows. Tables 1 to 61 show the evaluation results.

### Drive Voltage

The voltage (unit: V) when electric current was applied between the anode and the cathode so that the current density was 10 mA/cm² was measured.

### External Quantum Efficiency EQE

Voltage was applied on the organic EL devices so that a current density was 10 mA/cm², where spectral radiance spectrum was measured by a spectroradiometer (CS-2000 manufactured by Konica Minolta, Inc.). The external quantum efficiency EQE (unit: %) was calculated based on the obtained spectral-radiance spectra, assuming that the spectra was provided under a Lambertian radiation.

### Lifetime LT90

Voltage was applied on the resultant organic EL devices so that a current density was 50 mA/cm², where a time (LT90 (unit: hr)) elapsed before a luminance intensity was reduced to 90% of the initial luminance intensity was measured.

### Lifetime LT95

Voltage was applied on the resultant organic EL devices so that a current density was 50 mA/cm², where a time (LT95 (unit: hr)) elapsed before a luminance intensity was reduced to 95% of the initial luminance intensity was measured.

Maximum Peak Wavelength λp of Light Emitted from Device when Device is Driven

Voltage was applied on the organic EL devices so that a current density of the organic EL device was 10 mA/cm², where spectral radiance spectrum was measured by a spectroradiometer CS-2000 (manufactured by Konica Minolta, Inc.). The main peak wavelength λp (unit: nm) was calculated based on the obtained spectral radiance spectrum.

Maximum Peak Wavelength λp of Light Emitted from Emitting Layer when Device is Driven

For a main peak wavelength λp₁ of light emitted from the first emitting layer when the organic EL device is driven, the organic EL device was manufactured by using the same material as the first emitting layer for the second emitting layer, and voltage is applied on the organic EL device so that a current density became 10 mA/cm², where spectral radiance spectrum was measured by a spectroradiometer CS-2000 (manufactured by Konica Minolta, Inc.). The main peak wavelength λp₁ (unit: nm) was calculated based on the obtained spectral radiance spectrum.

For a main peak wavelength λp₂ of light emitted from the second emitting layer when the organic EL device was driven, the organic EL device was manufactured by using the same material as the second emitting layer for the first emitting layer, and voltage was applied on the organic EL device so that a current density became 10 mA/cm², where spectral radiance spectrum was measured by a spectroradiometer CS-2000 (manufactured by Konica Minolta, Inc.). The main peak wavelength λp₂ (unit: nm) was calculated based on the obtained spectral radiance spectrum.

### CIE1931 Chromaticity

Voltage was applied on the organic EL devices so that a current density was 10 mA/cm², where spectral radiance spectrum was measured by a spectroradiometer (CS-2000 manufactured by Konica Minolta, Inc.).

CIEx and CIEy were calculated based on the obtained spectral radiance spectrum.

TABLE 1

| | First Emitting Layer | | | | | Second Emitting Layer | | | | | | | |
|---|---|---|---|---|---|---|---|---|---|---|---|---|---|
| | | | | First | | | | | Second | | | | |
| | | | | Dopant | | | | | Dopant | | | | |
| | First Host Material | | | Material | Film | Second Host Material | | | Material | Film | | | |
| | Compound | | | Compound | Thick- | Compound | | | Compound | Thick- | EQE | LT90 | λp |
| | Name | S₁[ev] | T₁[ev] | Name | ness | Name | S₁[ev] | T₁[ev] | Name | ness | [%] | [hr] | [nm] |
| Example 1 | BH1 | 3.12 | 2.10 | BD1 | 5 | BH2 | 3.01 | 1.87 | BD1 | 20 | 10.6 | 600 | 461 |
| Comparative | BH1 | 3.12 | 2.10 | BD1 | 25 | — | — | — | — | — | 7.6 | 360 | 462 |
| Ex. 1 | | | | | | | | | | | | | |
| Comparative | — | — | — | — | — | BH2 | 3.01 | 1.87 | BD1 | 25 | 9.9 | 363 | 460 |
| Ex. 2 | | | | | | | | | | | | | |

TABLE 2

| | First Emitting Layer | | | | | Second Emitting Layer | | | | | | | |
|---|---|---|---|---|---|---|---|---|---|---|---|---|---|
| | First | | | First | | Second | | | Second | | | | |
| | Host | | | Dopant | | Host | | | Dopant | | Drive | | |
| | Material | | | Material | Film | Material | | | Material | Film | Voltage V | | |
| | Compound | S₁ | T₁ | Compound | Thickness | Compound | S₁ | T₁ | Compound | Thickness | @10 mA | λp | EQE |
| | Name | [ev] | [ev] | Name | [nm] | Name | [ev] | [ev] | Name | [nm] | [V] | [nm] | [%] |
| Example A2 | BH2 | 3.01 | 1.87 | BD1 | 10 | BH4 | 2.93 | 1.82 | BD1 | 15 | 3.72 | 460 | 10.7 |
| Example A3 | BH2-3 | 3.01 | 1.87 | BD1 | 5 | BH4 | 2.93 | 1.82 | BD1 | 20 | 3.77 | 460 | 10.8 |
| Example A4 | BH2-3 | 3.01 | 1.87 | BD1 | 10 | BH4 | 2.93 | 1.82 | BD1 | 15 | 3.75 | 460 | 10.7 |
| Comparative | BH2 | 3.01 | 1.87 | BD1 | 25 | — | — | — | — | — | 4.03 | 460 | 9.6 |
| Ex. A3 | | | | | | | | | | | | | |
| Comparative | — | — | — | — | — | BH4 | 2.93 | 1.82 | BD1 | 25 | 3.72 | 460 | 9.9 |
| Ex. A4 | | | | | | | | | | | | | |
| Comparative | BH2-3 | 3.01 | 1.87 | BD1 | 25 | — | — | — | — | — | 4.11 | 460 | 9.5 |
| Ex. A5 | | | | | | | | | | | | | |

Since the organic EL devices in Examples 1 and A2 to A4 include the first emitting layer and the second emitting layer including the respective host materials satisfying the relationship of the numerical formula (Numerical Formula 1), the organic EL devices in Examples 1 and A2 to A4 improved EQE more than the organic EL devices in Comparatives Examples 1, 2, and A3 to A5.

### Examples 2 to 20

The organic EL devices of Examples 2 and 20 were manufactured in the same manner as that of Example 1 except that the compound BH1 (first host material) in the first emitting layer was replaced with the compounds shown in Table 3.

### Comparative Examples 3 to 21

The organic EL devices of Comparative Examples 3 and 21 were manufactured in the same manner as that of Comparative Example 1 except that the compound BH1 (first host material) in the first emitting layer was replaced with the first compounds shown in Table 4.

TABLE 3

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Example 1 | BH1 | BD1 | 5 | BH2 | BD1 | 20 | 3.47 | 10.6 | 255 |
| Example 2 | BH1-2 | BD1 | 5 | BH2 | BD1 | 20 | 3.47 | 10.2 | 205 |
| Example 3 | BH1-3 | BD1 | 5 | BH2 | BD1 | 20 | 3.56 | 10.5 | 268 |
| Example 4 | BH1-4 | BD1 | 5 | BH2 | BD1 | 20 | 3.56 | 10.7 | 222 |
| Example 5 | BH1-5 | BD1 | 5 | BH2 | BD1 | 20 | 3.64 | 10.7 | 251 |
| Example 6 | BH1-6 | BD1 | 5 | BH2 | BD1 | 20 | 3.65 | 10.6 | 224 |
| Example 7 | BH1-7 | BD1 | 5 | BH2 | BD1 | 20 | 3.63 | 10.4 | 239 |
| Example 8 | BH1-8 | BD1 | 5 | BH2 | BD1 | 20 | 3.62 | 10.4 | 224 |
| Example 9 | BH1-9 | BD1 | 5 | BH2 | BD1 | 20 | 3.70 | 10.8 | 249 |
| Example 10 | BH1-10 | BD1 | 5 | BH2 | BD1 | 20 | 3.34 | 10.4 | 216 |
| Example 11 | BH1-11 | BD1 | 5 | BH2 | BD1 | 20 | 3.48 | 10.8 | 275 |
| Example 12 | BH1-12 | BD1 | 5 | BH2 | BD1 | 20 | 3.39 | 10.6 | 212 |
| Example 13 | BH1-13 | BD1 | 5 | BH2 | BD1 | 20 | 3.51 | 10.6 | 231 |
| Example 14 | BH1-14 | BD1 | 5 | BH2 | BD1 | 20 | 3.36 | 10.4 | 198 |
| Example 15 | BH1-15 | BD1 | 5 | BH2 | BD1 | 20 | 3.43 | 10.5 | 190 |
| Example 16 | BH1-16 | BD1 | 5 | BH2 | BD1 | 20 | 3.30 | 10.5 | 192 |
| Example 17 | BH1-17 | BD1 | 5 | BH2 | BD1 | 20 | 3.38 | 10.2 | 185 |
| Example 18 | BH1-18 | BD1 | 5 | BH2 | BD1 | 20 | 3.41 | 10.6 | 204 |
| Example 19 | BH1-19 | BD1 | 5 | BH2 | BD1 | 20 | 3.39 | 10.3 | 191 |
| Example 20 | R-BH1 | BD1 | 5 | BH2 | BD1 | 20 | 3.91 | 10.1 | — |

TABLE 4

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nml | Compound | Compound | [nm] | [V] | [%] | [hr] |
| Comparative | BH1 | BD1 | 25 | — | — | — | — | 7.6 | 65 |
| Ex. 1 | | | | | | | | | |
| Comparative | — | — | — | BH2 | BD1 | 25 | — | 9.9 | 167 |
| Ex. 2 | | | | | | | | | |
| Comparative | BH1-2 | BD1 | 25 | — | — | — | — | 7.2 | 59 |
| Ex. 3 | | | | | | | | | |
| Comparative | BH1-3 | BD1 | 25 | — | — | — | — | 7.4 | 71 |
| Ex. 4 | | | | | | | | | |
| Comparative | BH1-4 | BD1 | 25 | — | — | — | — | 7.8 | 70 |
| Ex. 5 | | | | | | | | | |
| Comparative | BH1-5 | BD1 | 25 | — | — | — | — | 7.5 | 62 |
| Ex. 6 | | | | | | | | | |
| Comparative | BH1-6 | BD1 | 25 | — | — | — | — | 7.4 | 60 |
| Ex. 7 | | | | | | | | | |
| Comparative | BH1-7 | BD1 | 25 | — | — | — | — | 7.3 | 53 |
| Ex. 8 | | | | | | | | | |
| Comparative | BH1-8 | BD1 | 25 | — | — | — | — | 7.4 | 55 |
| Ex. 9 | | | | | | | | | |
| Comparative | BH1-9 | BD1 | 25 | — | — | — | — | 7.5 | 67 |
| Ex. 10 | | | | | | | | | |
| Comparative | BH1-10 | BD1 | 25 | — | — | — | — | 7.1 | 51 |
| Ex. 11 | | | | | | | | | |
| Comparative | BH1-11 | BD1 | 25 | — | — | — | — | 7.8 | 81 |
| Ex. 12 | | | | | | | | | |
| Comparative | BH1-12 | BD1 | 25 | — | — | — | — | 7.0 | 48 |
| Ex. 13 | | | | | | | | | |
| Comparative | BH1-13 | BD1 | 25 | — | — | — | — | 7.1 | 53 |
| Ex. 14 | | | | | | | | | |
| Comparative | BH1-14 | BD1 | 25 | — | — | — | — | 6.9 | 56 |
| Ex. 15 | | | | | | | | | |
| Comparative | BH1-15 | BD1 | 25 | — | — | — | — | 7.1 | 59 |
| Ex. 16 | | | | | | | | | |
| Comparative | BH1-16 | BD1 | 25 | — | — | — | — | 7.0 | 62 |
| Ex. 17 | | | | | | | | | |
| Comparative | BH1-17 | BD1 | 25 | — | — | — | — | 6.7 | 53 |
| Ex. 18 | | | | | | | | | |
| Comparative | BH1-18 | BD1 | 25 | — | — | — | — | 7.1 | 62 |
| Ex. 19 | | | | | | | | | |
| Comparative | BH1-19 | BD1 | 25 | — | — | — | — | 6.9 | 43 |
| Ex. 20 | | | | | | | | | |
| Comparative | BH1-20 | BD1 | 25 | — | — | — | — | 6.5 | 21 |
| Ex. 21 | | | | | | | | | |

### Example 21

The organic EL devices of Example 21 was manufactured in the same manner as that of Reference Example 1 except that the compound BH2 (second host material) in the second emitting layer was replaced with the compound shown in Table 5.

### Examples 22 and 23

The organic EL devices of Examples 22 and 23 were manufactured in the same manner as that of Example 1 except that the compound BH1 (first host material) in the first emitting layer and the compound BH2 (second host material) in the second emitting layer were replaced with the compounds shown in Table 5.

### Comparative Example 22

The organic EL device of Comparative Example 22 was manufactured in the same manner as that of Comparative Example 2 except that the compound BH2 (second host material) in the second emitting layer was replaced with the compound shown in Table 5.

TABLE 5

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Example 21 | BH1 | BD1 | 5 | BH2-2 | BD1 | 20 | 3.96 | 9.8 | 192 |
| Example 22 | R-BH1 | BD1 | 5 | BH2-2 | BD1 | 20 | 4.40 | 9.4 | — |
| Example 23 | R-BH2 | BD1 | 5 | BH2-2 | BD1 | 20 | 4.68 | 9.5 | — |
| Comparative | BH1 | BD1 | 25 | — | — | — | — | 7.6 | 65 |
| Ex. 1 | | | | | | | | | |
| Comparative | — | — | — | BH2-2 | BD1 | 25 | — | 9.2 | 115 |
| Ex. 22 | | | | | | | | | |

### Example 24

The organic EL device of Example 24 was manufactured in the same manner as that of Example 1 except that the compound BH2 (second host material) in the second emitting layer was replaced with the compound shown in Table 6.

### Examples 25 and 26

The organic EL devices of Examples 25 and 26 were manufactured in the same manner as that of Example 1 except that the compound BH1 (first host material) in the first emitting layer and the compound BH2 (second host material) in the second emitting layer were replaced with the compounds shown in Table 6.

### Comparative Example 23

The organic EL device of Comparative Example 23 was manufactured in the same manner as that of Comparative Example 2 except that the compound BH2 (second host material) in the second emitting layer was replaced with the compound shown in Table 6.

TABLE 6

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Example 24 | BH1 | BD1 | 5 | BH2-3 | BD1 | 20 | 3.54 | 10.6 | 278 |
| Example 25 | R-BH1 | BD1 | 5 | BH2-3 | BD1 | 20 | 3.98 | 10.1 | — |
| Example 26 | R-BH2 | BD1 | 5 | BH2-3 | BD1 | 20 | 4.26 | 10.2 | — |
| Comparative | BH1 | BD1 | 25 | — | — | — | — | 7.6 | 65 |
| Ex. 1 | | | | | | | | | |
| Comparative | — | — | — | BH2-3 | BD1 | 25 | — | 9.9 | 182 |
| Ex. 23 | | | | | | | | | |

### Example 27

The organic EL device of Example 27 was manufactured in the same manner as that of Example 1 except that the compound BH2 (second host material) in the second emitting layer were replaced with the compound shown in Table 7.

### Examples 28 and 29

The organic EL devices of Examples 28 and 29 were manufactured in the same manner as that of Example 1 except that the compound BH1 (first host material) in the first emitting layer and the compound BH2 (second host material) in the second emitting layer were replaced with the compounds shown in Table 7.

### Comparative Example 24

The organic EL device of Comparative Example 24 was manufactured in the same manner as that of Comparative Example 2 except that the compound BH2 (second host material) in the second emitting layer was replaced with the compound shown in Table 7.

TABLE 7

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Example 27 | BH1 | BD1 | 5 | BH2-4 | BD1 | 20 | 3.26 | 8.1 | 272 |
| Example 28 | R-BH1 | BD1 | 5 | BH2-4 | BD1 | 20 | 3.70 | 7.9 | — |
| Example 29 | R-BH2 | BD1 | 5 | BH2-4 | BD1 | 20 | 3.98 | 7.9 | — |
| Comparative | BH1 | BD1 | 25 | — | — | — | — | 7.6 | 65 |
| Ex. 1 | | | | | | | | | |
| Comparative | — | — | — | BH2-4 | BD1 | 25 | — | 7.7 | 114 |
| Ex. 24 | | | | | | | | | |

### Example 30

The organic EL device of Example 30 was manufactured in the same manner as that of Example 1 except that the compound BH2 (second host material) in the second emitting layer was replaced with the compound shown in Table 8.

### Examples 31 and 32

The organic EL devices of Examples 31 and 32 were manufactured in the same manner as that of Example 1 except that the compound BH1 (first host material) in the first emitting layer and the compound BH2 (second host material) in the second emitting layer were replaced with the compounds shown in Table 8.

### Comparative Example 25

The organic EL device of Comparative Example 25 was manufactured in the same manner as that of Comparative Example 2 except that the compound BH2 (second host material) in the second emitting layer was replaced with the compound shown in Table 8.

TABLE 8

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Example 30 | BH1 | BD1 | 5 | BH2-5 | BD1 | 20 | 3.76 | 8.0 | 196 |
| Example 31 | R-BH1 | BD1 | 5 | BH2-5 | BD1 | 20 | 4.20 | 7.8 | — |
| Example 32 | R-BH2 | BD1 | 5 | BH2-5 | BD1 | 20 | 4.48 | 7.8 | — |
| Comparative | BH1 | BD1 | 25 | — | — | — | — | 7.6 | 65 |
| Ex. 1 | | | | | | | | | |
| Comparative | — | — | — | BH2-5 | BD1 | 25 | — | 7.6 | 92 |
| Ex. 25 | | | | | | | | | |

### Example 33

The organic EL device of Example 33 was manufactured in the same manner as that of Example 1 except that the compound BH2 (second host material) in the second emitting layer was replaced with the compound shown in Table 9.

### Examples 34 and 35

The organic EL devices of Examples 34 and 35 were manufactured in the same manner as that of Example 1 except that the compound BH1 (first host material) in the first emitting layer and the compound BH2 (second host material) in the second emitting layer were replaced with the compounds shown in Table 9.

### Comparative Example 26

The organic EL device according to Comparative Example 26 was manufactured in the same manner as that of Reference Example 2 except that the compound BH2 (second host material) in the second emitting layer was replaced with the compound listed in Table 9.

TABLE 9

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Example 33 | BH1 | BD1 | 5 | BH2-6 | BD1 | 20 | 3.14 | 10.5 | 198 |
| Example 34 | R-BH1 | BD1 | 5 | BH2-6 | BD1 | 20 | 3.58 | 8.2 | — |
| Example 35 | R-BH2 | BD1 | 5 | BH2-6 | BD1 | 20 | 3.86 | 8.2 | — |
| Comparative | BH1 | BD1 | 25 | — | — | — | — | 7.6 | 65 |
| Ex. 1 | | | | | | | | | |
| Comparative | — | — | — | BH2-6 | BD1 | 25 | — | 8.0 | 71 |
| Ex. 26 | | | | | | | | | |

### Example 36

The organic EL devices of Example 36 was manufactured in the same manner as that of Example 1 except that the compound BH2 (second host material) in the second emitting layer were replaced with the compound shown in Table 6.

### Examples 37 and 38

The organic EL devices of Examples 37 and 38 were manufactured in the same manner as that of Example 1 except that the compound BH1 (first host material) in the first emitting layer and the compound BH2 (second host material) in the second emitting layer were replaced with the compounds shown in Table 10.

### Comparative Example 27

The organic EL devices according to Comparative Example 27 was manufactured in the same manner as that of Comparative Example 2 except that the compound BH2 (second host material) in the second emitting layer was replaced with the compound shown in Table 10.

TABLE 10

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Example 36 | BH1 | BD1 | 5 | BH2-7 | BD1 | 20 | 3.21 | 10.7 | 217 |
| Example 37 | R-BH1 | BD1 | 5 | BH2-7 | BD1 | 20 | 3.65 | 8.0 | — |
| Example 38 | R-BH2 | BD1 | 5 | BH2-7 | BD1 | 20 | 3.93 | 8.0 | — |
| Comparative | BH1 | BD1 | 25 | — | — | — | — | 7.6 | 65 |
| Ex. 1 | | | | | | | | | |
| Comparative | — | — | — | BH2-7 | BD1 | 25 | — | 7.8 | 106 |
| Ex. 27 | | | | | | | | | |

### Example 39

The organic EL device of Example 39 was manufactured in the same manner as that of Example 1 except the compound BH2 (second host material) in the second emitting layer were replaced with the compound shown in Table 11.

### Examples 40 and 41

The organic EL devices of Examples 40 and 41 were manufactured in the same manner as that of Example 1 except that the compound BH1 (first host material) in the first emitting layer and the compound BH2 (second host material) in the second emitting layer were replaced with the compounds shown in Table 11.

### Comparative Example 28

The organic EL device of Comparative Example 28 was manufactured in the same manner as that of Comparative Example 2 except that the compound BH2 (second host material) in the second emitting layer was replaced with the compound shown in Table 11.

TABLE 11

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Example 39 | BH1 | BD1 | 5 | BH2-8 | BD1 | 20 | 3.39 | 9.2 | 192 |
| Example 40 | R-BH1 | BD1 | 5 | BH2-8 | BD1 | 20 | 3.83 | 8.0 | — |
| Example 41 | R-BH2 | BD1 | 5 | BH2-8 | BD1 | 20 | 4.11 | 8.0 | — |
| Comparative | BH1 | BD1 | 25 | — | — | — | — | 7.6 | 65 |
| Ex. 1 | | | | | | | | | |
| Comparative | — | — | — | BH2-8 | BD1 | 25 | — | 7.8 | 74 |
| Ex. 28 | | | | | | | | | |

### Example 42

The organic EL device of Example 42 was manufactured in the same manner as that of Example 1 except that the compound BH2 (second host material) in the second emitting layer were replaced with the compound shown in Table 12.

### Examples 43 and 44

The organic EL devices of Examples 43 and 44 were manufactured in the same manner as that of Example 1 except that the compound BH1 (first host material) in the first emitting layer and the compound BH2 (second host material) in the second emitting layer were replaced with the compounds shown in Table 12.

### Comparative Example 29

The organic EL device of Comparative Example 29 was manufactured in the same manner as that of Comparative Example 2 except that the compound BH2 (second host material) in the second emitting layer was replaced with the compound shown in Table 12.

TABLE 12

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Example 42 | BH1 | BD1 | 5 | BH2-9 | BD1 | 20 | 3.56 | 10.5 | 300 |
| Example 43 | R-BH1 | BD1 | 5 | BH2-9 | BD1 | 20 | 4.00 | 10.0 | — |
| Example 44 | R-BH2 | BD1 | 5 | BH2-9 | BD1 | 20 | 4.28 | 10.1 | — |
| Comparative | BH1 | BD1 | 25 | — | — | — | — | 7.6 | 65 |
| Ex. 1 | | | | | | | | | |
| Comparative | — | — | — | BH2-9 | BD1 | 25 | — | 9.8 | 195 |
| Ex. 29 | | | | | | | | | |

### Example 45

The organic EL device of Example 45 was manufactured in the same manner as that of Example 1 except that the compound BD1 in the first emitting layer and the compound BD1 in the second emitting layer were replaced with the compound shown in Table 13

### Examples 46 and 47

The organic EL devices of Examples 46 and 47 were manufactured in the same manner as that of Example 1 except that the compound BH1 (first host material) and the compound BD1 in the first emitting layer and the compound BD1 in the second emitting layer were replaced with the compounds shown in Table 13.

### Comparative Example 30

The organic EL device of Comparative Example 30 was manufactured in the same manner as that of Comparative Example 1 except that the compound BD1 in the first emitting layer was replaced with the compound shown in Table 13.

### Comparative Example 31

The organic EL device of Comparative Example 31 was manufactured in the same manner as that of Comparative Example 2 except that the compound BD1 in the second emitting layer was replaced with the compound shown in Table 13

TABLE 13

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Example 45 | BH1 | BD2 | 5 | BH2 | BD2 | 20 | 3.57 | 9.7 | 203 |
| Example 46 | R-BH1 | BD2 | 5 | BH2 | BD2 | 20 | 4.01 | 9.3 | — |
| Example 47 | R-BH2 | BD2 | 5 | BH2 | BD2 | 20 | 4.29 | 9.4 | — |
| Comparative | BH1 | BD2 | 25 | — | — | — | — | 7.0 | 51 |
| Ex. 30 | | | | | | | | | |
| Comparative | — | — | — | BH2 | BD2 | 25 | — | 9.1 | 120 |
| Ex. 31 | | | | | | | | | |

### Example 48

The organic EL device of Example 48 was manufactured in the same manner as that of Example 1 except that the compound BD1 in the first emitting layer and the compound BD1 in the second emitting layer were replaced with the compounds shown in Table 14.

### Examples 49 and 50

The organic EL devices of Examples 49 and 50 were manufactured in the same manner as that of Example 1 except that the compound BH1 (first host material) and the compound BD1 in the first emitting layer and the compound BD1 in the second emitting layer were replaced with the compounds shown in Table 14.

### Comparative Example 32

The organic EL device of Comparative Example 32 was manufactured in the same manner as that of Example 1 except that the compound BD1 in the first emitting layer was replaced with the compound shown in Table 14.

### Comparative Example 33

The organic EL device of Comparative Example 33 was manufactured in the same manner as that of Comparative Example 2 except that the compound BD1 in the second emitting layer was replaced with the compound shown in Table 14.

TABLE 14

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Example 48 | BH1 | BD3 | 5 | BH2 | BD3 | 20 | 3.51 | 10.2 | 167 |
| Example 49 | R-BH1 | BD3 | 5 | BH2 | BD3 | 20 | 3.95 | 9.7 | — |
| Example 50 | R-BH2 | BD3 | 5 | BH2 | BD3 | 20 | 4.23 | 9.8 | — |
| Comparative | BH1 | BD3 | 25 | — | — | — | — | 7.4 | 46 |
| Ex. 32 | | | | | | | | | |
| Comparative | — | — | — | BH2 | BD3 | 25 | — | 9.5 | 103 |
| Ex. 33 | | | | | | | | | |

### Examples 51 to 69

The organic EL devices of Examples 51 to 69 were manufactured in the same manner as that of Example 1 except that the compound BH1 (first host material) in the first emitting layer was replaced with the compound shown in Table 15.

TABLE 15

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Example 51 | BH1-23 | BD1 | 5 | BH2 | BD1 | 20 | 10.2 | 198 |
| Example 52 | BH1-26 | BD1 | 5 | BH2 | BD1 | 20 | 10.3 | 214 |
| Example 53 | BH1-27 | BD1 | 5 | BH2 | BD1 | 20 | 10.6 | 239 |
| Example 54 | BH1-28 | BD1 | 5 | BH2 | BD1 | 20 | 10.5 | 222 |
| Example 55 | BH1-32 | BD1 | 5 | BH2 | BD1 | 20 | 10.4 | 207 |
| Example 56 | BH1-33 | BD1 | 5 | BH2 | BD1 | 20 | 10.3 | 205 |
| Example 57 | BH1-34 | BD1 | 5 | BH2 | BD1 | 20 | 10.5 | 213 |
| Example 58 | BH1-35 | BD1 | 5 | BH2 | BD1 | 20 | 10.4 | 198 |
| Example 59 | BH1-40 | BD1 | 5 | BH2 | BD1 | 20 | 10.4 | 221 |
| Example 60 | BH1-41 | BD1 | 5 | BH2 | BD1 | 20 | 10.7 | 248 |
| Example 61 | BH1-42 | BD1 | 5 | BH2 | BD1 | 20 | 10.5 | 232 |
| Example 62 | BH1-43 | BD1 | 5 | BH2 | BD1 | 20 | 10.6 | 211 |
| Example 63 | BH1-44 | BD1 | 5 | BH2 | BD1 | 20 | 10.5 | 205 |
| Example 64 | BH1-45 | BD1 | 5 | BH2 | BD1 | 20 | 10.4 | 230 |
| Example 65 | BH1-46 | BD1 | 5 | BH2 | BD1 | 20 | 10.8 | 249 |
| Example 66 | BH1-47 | BD1 | 5 | BH2 | BD1 | 20 | 10.6 | 217 |
| Example 67 | BH1-48 | BD1 | 5 | BH2 | BD1 | 20 | 10.6 | 243 |
| Example 68 | BH1-49 | BD1 | 5 | BH2 | BD1 | 20 | 10.7 | 268 |
| Example 69 | R-BH3 | BD1 | 5 | BH2 | BD1 | 20 | 10.1 | 183 |

### Comparative Examples 34 to 51

The organic EL devices of Comparative Examples 34 to 51 were manufactured in the same manner as that of Comparative Example 1 except that the compound BH1 (first host material) in the first emitting layer was replaced with the compound shown in Table 16.

TABLE 16

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Comparative | BH1-23 | BD1 | 25 | — | — | — | 6.3 | 50 |
| Ex. 34 | | | | | | | | |
| Comparative | BH1-26 | BD1 | 25 | — | — | — | 6.6 | 78 |
| Ex. 35 | | | | | | | | |
| Comparative | BH1-27 | BD1 | 25 | — | — | — | 6.7 | 81 |
| Ex. 36 | | | | | | | | |
| Comparative | BH1-28 | BD1 | 25 | — | — | — | 6.5 | 72 |
| Ex. 37 | | | | | | | | |
| Comparative | BH1-32 | BD1 | 25 | — | — | — | 6.1 | 49 |
| Ex. 38 | | | | | | | | |
| Comparative | BH1-33 | BD1 | 25 | — | — | — | 6.2 | 55 |
| Ex. 39 | | | | | | | | |
| Comparative | BH1-34 | BD1 | 25 | — | — | — | 6.2 | 57 |
| Ex. 40 | | | | | | | | |
| Comparative | BH1-35 | BD1 | 25 | — | — | — | 6.0 | 49 |
| Ex. 41 | | | | | | | | |
| Comparative | BH1-40 | BD1 | 25 | — | — | — | 6.2 | 68 |
| Ex. 42 | | | | | | | | |
| Comparative | BH1-41 | BD1 | 25 | — | — | — | 6.6 | 91 |
| Ex. 43 | | | | | | | | |
| Comparative | BH1-42 | BD1 | 25 | — | — | — | 6.4 | 85 |
| Ex. 44 | | | | | | | | |
| Comparative | BH1-43 | BD1 | 25 | — | — | — | 6.4 | 72 |
| Ex. 45 | | | | | | | | |
| Comparative | BH1-44 | BD1 | 25 | — | — | — | 6.4 | 77 |
| Ex. 46 | | | | | | | | |
| Comparative | BH1-45 | BD1 | 25 | — | — | — | 6.2 | 81 |
| Ex. 47 | | | | | | | | |
| Comparative | BH1-46 | BD1 | 25 | — | — | — | 6.3 | 94 |
| Ex. 48 | | | | | | | | |
| Comparative | BH1-47 | BD1 | 25 | — | — | — | 6.2 | 67 |
| Ex. 49 | | | | | | | | |
| Comparative | BH1-48 | BD1 | 25 | — | — | — | 6.1 | 64 |
| Ex. 50 | | | | | | | | |
| Comparative | BH1-49 | BD1 | 25 | — | — | — | 6.8 | 97 |
| Ex. 51 | | | | | | | | |
| Comparative | — | — | — | BH2 | BD1 | 25 | 9.9 | 167 |
| Ex. 2 | | | | | | | | |

### Manufacture 2 of Organic EL Device

Organic EL devices were manufactured and evaluated as follows.

### Example 70

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT3 was vapor-deposited to form an 80-nm-thick first hole transporting layer (HT).

After the formation of the first hole transporting layer, the compound HT4 was vapor-deposited to form a 10-nm-thick second hole transporting layer (also referred to as an electron blocking layer (EBL)).

A compound BH1-21 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET4 was vapor-deposited on the second emitting layer to form a 10-nm-thick first electron transporting layer (also referred to as a hole blocking layer (HBL)).

The compound ET2 was vapor-deposited on the first electron transporting layer to form a 15-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 70 is roughly shown as follows.

$$ITO ( 130 ) / HA 1 ( 5 ) / HT 3 ( 80 ) / HT 4 ( 10 ) / BH 1 - 21 : BD 1 ( 5 , 98 \% : 2 \% ) / BH 2 : BD 1 ( 20 , 98 \% : 2 \% ) / ET 4 ( 10 ) / ET 2 ( 15 ) / LiF ( 1 ) / Al ( 80 )$$

The numerals in parentheses represent a film thickness (unit: nm).

The numerals (98%:2%) represented by percentage in the same parentheses indicate a ratio (mass %) between the host material (compound BH1-21 or BH2) and the compound BD1 in the first emitting layer or the second emitting layer. Similar notations apply to the description below.

### Examples 71 to 78

The organic EL devices of Examples 71 to 78 were manufactured in the same manner as that of Example 70 except that the compound BH1-21 (first host material) in the first emitting layer was replaced with the compounds shown in Table 17.

### Comparative Examples 52 to 59

The organic EL devices of Comparative Examples 52 to 59 were manufactured in the same manner as that of Example 70 except that a 25-nm-thick first emitting layer was formed as the emitting layer, the first electron transporting layer was formed on the first emitting layer without forming the second emitting layer, and the first compound (first host material) in the first emitting layer was replaced with the first compounds listed in Table 17.

### Comparative Example 60

The organic EL device according to Comparative Example 60 was manufactured in the same manner as that of Example 70 except that a 25-nm-thick second emitting layer was formed on the second hole transporting layer without forming the first emitting layer as shown in Table 17.

TABLE 17

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Example 70 | BH1-21 | BD1 | 5 | BH2 | BD1 | 20 | 3.40 | 8.7 | 160 |
| Example 71 | BH1-22 | BD1 | 5 | BH2 | BD1 | 20 | 3.46 | 9.0 | 225 |
| Example 72 | BH1-24 | BD1 | 5 | BH2 | BD1 | 20 | 3.27 | 8.4 | 79 |
| Example 73 | BH1-25 | BD1 | 5 | BH2 | BD1 | 20 | 3.35 | 8.7 | 174 |
| Example 74 | BH1-36 | BD1 | 5 | BH2 | BD1 | 20 | 3.39 | 8.5 | 125 |
| Example 75 | BH1-37 | BD1 | 5 | BH2 | BD1 | 20 | 3.44 | 8.8 | 135 |
| Example 76 | BH1-50 | BD1 | 5 | BH2 | BD1 | 20 | 3.42 | 8.5 | 111 |
| Example 77 | BH1-51 | BD1 | 5 | BH2 | BD1 | 20 | 3.31 | 8.4 | 105 |
| Example 78 | R-BH3 | BD1 | 5 | BH2 | BD1 | 20 | 3.53 | 7.9 | 36 |
| Comparative | BH1-21 | BD1 | 25 | — | — | — | — | 6.2 | 32 |
| Ex. 52 | | | | | | | | | |
| Comparative | BH1-22 | BD1 | 25 | — | — | — | — | 6.4 | 45 |
| Ex. 53 | | | | | | | | | |
| Comparative | BH1-24 | BD1 | 25 | — | — | — | — | 6.0 | 13 |
| Ex. 54 | | | | | | | | | |
| Comparative | BH1-25 | BD1 | 25 | — | — | — | — | 6.2 | 25 |
| Ex. 55 | | | | | | | | | |
| Comparative | BH1-36 | BD1 | 25 | — | — | — | — | 6.1 | 25 |
| Ex. 56 | | | | | | | | | |
| Comparative | BH1-37 | BD1 | 25 | — | — | — | — | 6.3 | 27 |
| Ex. 57 | | | | | | | | | |
| Comparative | BH1-50 | BD1 | 25 | — | — | — | — | 6.1 | 21 |
| Ex. 58 | | | | | | | | | |
| Comparative | BH1-51 | BD1 | 25 | — | — | — | — | 6.0 | 19 |
| Ex. 59 | | | | | | | | | |
| Comparative | — | — | — | BH2 | BD1 | 25 | — | 7.7 | 56 |
| Ex. 60 | | | | | | | | | |

### Manufacture 3 of Organic EL Device

Organic EL devices were manufactured and evaluated as follows.

### Example 79

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT3 was vapor-deposited to form an 80-nm-thick first hole transporting layer (HT).

After the formation of the first hole transporting layer, the compound HT4 was vapor-deposited to form a 10-nm-thick second hole transporting layer (also referred to as an electron blocking layer (EBL)).

A compound BH1-29 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

A compound ET3 was vapor-deposited on the second emitting layer to form a 10-nm-thick first electron transporting layer (also referred to as a hole blocking layer (HBL)).

The compound ET2 was vapor-deposited on the first electron transporting layer to form a 15-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 79 is roughly shown as follows.

$$ITO ( 130 ) / HA 1 ( 5 ) / HT 3 ( 80 ) / HT 4 ( 10 ) / BH 1 - 29 : BD 1 ( 5 , 98 \% : 2 \% ) / BH 2 : BD 1 ( 20 , 98 \% : 2 \% ) / ET 3 ( 10 ) / ET 2 ( 15 ) / LiF ( 1 ) / Al ( 80 )$$

The numerals in parentheses represent a film thickness (unit: nm).

The numerals (98%:2%) represented by percentage in the same parentheses indicate a ratio (mass %) between the host material (compound BH1-29 or BH2) and the compound BD1 in the first emitting layer or the second emitting layer. Similar notations apply to the description below.

### Examples 80 to 90

The organic EL devices of Comparative Examples 80 to 90 were manufactured in the same manner as that of Example 79 except that the compound BH1-2 (first host material) in the first emitting layer was replaced with the first compounds shown in Table 18.

### Comparative Examples 61 to 71

Organic EL devices of Comparative Examples 61 to 71 were manufactured in the same manner as that of Example 79 except that a 25-nm thick first emitting layer was formed as the emitting layer, the first electron transporting layer was formed on the first emitting layer without forming the second emitting layer, and the first compound (first host material) in the first emitting layer was replaced by the first compound shown in Table 18.

### Comparative Example 72

The organic EL device of Comparative Example 72 was manufactured in the same manner as that of Example 79 except that a 25-nm-thick second emitting layer was formed on the second hole transporting layer without forming the first emitting layer as shown in Table 18.

TABLE 18

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Example 79 | BH1-29 | BD1 | 5 | BH2 | BD1 | 20 | 9.3 | 125 |
| Example 80 | BH1-30 | BD1 | 5 | BH2 | BD1 | 20 | 9.3 | 103 |
| Example 81 | BH1-31 | BD1 | 5 | BH2 | BD1 | 20 | 9.6 | 119 |
| Example 82 | BH1-38 | BD1 | 5 | BH2 | BD1 | 20 | 9.8 | 138 |
| Example 83 | BH1-39 | BD1 | 5 | BH2 | BD1 | 20 | 9.7 | 122 |
| Example 84 | BH1-52 | BD1 | 5 | BH2 | BD1 | 20 | 9.5 | 151 |
| Example 85 | BH1-53 | BD1 | 5 | BH2 | BD1 | 20 | 9.3 | 132 |
| Example 86 | BH1-54 | BD1 | 5 | BH2 | BD1 | 20 | 9.1 | 110 |
| Example 87 | BH1-55 | BD1 | 5 | BH2 | BD1 | 20 | 9.4 | 109 |
| Example 88 | BH1-56 | BD1 | 5 | BH2 | BD1 | 20 | 9.2 | 111 |
| Example 89 | BH1-57 | BD1 | 5 | BH2 | BD1 | 20 | 9.2 | 121 |
| Example 90 | R-BH3 | BD1 | 5 | BH2 | BD1 | 20 | 8.3 | 97 |
| Comparative | BH1-29 | BD1 | 25 | — | — | — | 6.7 | 61 |
| Ex. 61 | | | | | | | | |
| Comparative | BH1-30 | BD1 | 25 | — | — | — | 6.9 | 53 |
| Ex. 62 | | | | | | | | |
| Comparative | BH1-31 | BD1 | 25 | — | — | — | 6.4 | 51 |
| Ex. 63 | | | | | | | | |
| Comparative | BH1-38 | BD1 | 25 | — | — | — | 6.1 | 48 |
| Ex. 64 | | | | | | | | |
| Comparative | BH1-39 | BD1 | 25 | — | — | — | 6.1 | 45 |
| Ex. 65 | | | | | | | | |
| Comparative | BH1-52 | BD1 | 25 | — | — | — | 6.8 | 62 |
| Ex. 66 | | | | | | | | |
| Comparative | BH1-53 | BD1 | 25 | — | — | — | 6.8 | 54 |
| Ex. 67 | | | | | | | | |
| Comparative | BH1-54 | BD1 | 25 | — | — | — | 6.7 | 42 |
| Ex. 68 | | | | | | | | |
| Comparative | BH1-55 | BD1 | 25 | — | — | — | 6.7 | 59 |
| Ex. 69 | | | | | | | | |
| Comparative | BH1-56 | BD1 | 25 | — | — | — | 6.5 | 40 |
| Ex. 70 | | | | | | | | |
| Comparative | BH1-57 | BD1 | 25 | — | — | — | 6.2 | 34 |
| Ex. 71 | | | | | | | | |
| Comparative | — | — | — | BH2 | BD1 | 25 | 8.1 | 89 |
| Ex. 72 | | | | | | | | |

### Manufacture 4 of Organic EL Device

Organic EL devices were manufactured and evaluated as follows.

### Example 91

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HT5 and the compound HA2 were co-deposited on a surface provided with the transparent electrode line to cover the transparent electrode, thereby forming a 10-nm-thick hole injecting layer (HI). The ratios of the compound HT5 and the compound HA2 in the hole injecting layer were 97 mass % and 3 mass %, respectively.

After the formation of the hole injecting layer, the compound HT5 was vapor-deposited to form an 85-nm-thick first hole transporting layer (HT).

After the formation of the first hole transporting layer, the compound HT4 was vapor-deposited to form a 5-nm-thick second hole transporting layer (also referred to as an electron blocking layer (EBL)).

A compound BH1-61 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET3 was vapor-deposited on the second emitting layer to form a 5-nm-thick first electron transporting layer (also referred to as a hole blocking layer (HBL)).

The compound ET6 and the compound Liq were co-deposited on the first electron transporting layer (HBL) to form a 25-nm-thick electron transporting layer (ET). The ratios of the compound ET6 and the compound Liq in the electron transporting layer (ET) were 50 mass % and 50 mass %, respectively. Liq is an abbreviation of (8-quinolinolato)lithium.

Liq was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 91 is roughly shown as follows.

$$ITO ( 130 ) / HT 5 : HA 2 ( 10 , 97 \% : 3 \% ) / HT 5 ( 85 ) / HT 4 ( 5 ) / BH 1 - 61 : BD 1 ( 5 , 98 \% : 2 \% ) / BH 2 : BD 1 ( 20 , 98 \% : 2 \% ) / ET 3 ( 5 ) / ET 6 : Liq ( 25 , 50 \% : 50 \% ) / Liq ( 1 ) / Al ( 80 )$$

The numerals in parentheses represent a film thickness (unit: nm).

The numerals (97%:3%) represented by percentage in the same parentheses indicate a ratio (mass %) between the compound HT5 and the compound HA2 in the hole injecting layer, the numerals (98%:2%) represented by percentage in the same parentheses indicate a ratio (mass %) between the host material (compound BH1-61 or BH2) and the dopant material (compound BD1) in the first emitting layer or the second emitting layer, and the numerals (50%:50%) represented by percentage in the same parentheses indicate a ratio (mass %) between the compound ET6 and the compound Liq in the electron transporting layer (ET). Similar notations apply to the description below.

### Examples 92 to 95

The organic EL device of Examples 92 to 95 were manufactured in the same manner as that of Example 91 except that the compound BH1-61 (first host material) in the first emitting layer was replaced with the first compounds shown in Table 19.

### Comparative Examples 73 to 76

The organic EL devices of Comparative Examples 73 to 76 were manufactured in the same manner as that of Example 91 except that a 25-nm-thick first emitting layer was formed as the emitting layer, the first electron transporting layer was formed on the first emitting layer without forming the second emitting layer, and the first compound (first host material) in the first emitting layer was replaced with the first compounds shown in Table 19.

### Comparative Example 77

The organic EL device of Comparative Example 77 was manufactured in the same manner as that of Example 91 except that a 25-nm-thick second emitting layer was formed on the second hole transporting layer without forming the first emitting layer as shown in Table 19.

TABLE 19

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Example 91 | BH1-61 | BD1 | 5 | BH2 | BD1 | 20 | 9.2 | 128 |
| Example 92 | BH1-62 | BD1 | 5 | BH2 | BD1 | 20 | 9.7 | 153 |
| Example 93 | BH1-63 | BD1 | 5 | BH2 | BD1 | 20 | 9.5 | 144 |
| Example 94 | BH1-69 | BD1 | 5 | BH2 | BD1 | 20 | 9.0 | 110 |
| Example 95 | R-BH3 | BD1 | 5 | BH2 | BD1 | 20 | 8.8 | 101 |
| Comparative | BH1-61 | BD1 | 25 | — | — | — | 6.1 | 47 |
| Ex. 73 | | | | | | | | |
| Comparative | BH1-62 | BD1 | 25 | — | — | — | 6.4 | 64 |
| Ex. 74 | | | | | | | | |
| Comparative | BH1-63 | BD1 | 25 | — | — | — | 6.3 | 60 |
| Ex. 75 | | | | | | | | |
| Comparative | BH1-69 | BD1 | 25 | — | — | — | 5.9 | 19 |
| Ex. 76 | | | | | | | | |
| Comparative | — | — | — | BH2 | BD1 | 25 | 8.4 | 72 |
| Ex. 77 | | | | | | | | |

### Manufacture 5 of Organic EL Device

Organic EL devices were manufactured and evaluated as follows.

### Example 96

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HT3 and the compound HA2 were co-deposited on a surface provided with the transparent electrode line to cover the transparent electrode, thereby forming a 10-nm-thick hole injecting layer (HI). The ratios of the compound HT3 and the compound HA2 in the hole injecting layer were 97 mass % and 3 mass %, respectively.

After the formation of the hole injecting layer, the compound HT3 was vapor-deposited to form an 85-nm-thick first hole transporting layer (HT).

After the formation of the first hole transporting layer, the compound HT4 was vapor-deposited to form a 5-nm-thick second hole transporting layer (also referred to as an electron blocking layer (EBL)).

A compound BH1-75 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET3 was vapor-deposited on the second emitting layer to form a 5-nm-thick first electron transporting layer (also referred to as a hole blocking layer (HBL)).

The compound ET8 and the compound Liq were co-deposited on the first electron transporting layer (HBL) to form a 25-nm-thick electron transporting layer (ET). The ratios of the compound ET5 and the compound Liq in the electron transporting layer (ET) were 50 mass % and 50 mass %, respectively. Liq is an abbreviation of (8-quinolinolato)lithium.

Liq was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 96 is roughly shown as follows.

$$ITO ( 130 ) / HT 3 : HA 2 ( 10 , 97 \% : 3 \% ) / HT 3 ( 85 ) / HT 4 ( 5 ) / BH 1 - 75 : BD 1 ( 5 , 98 \% : 2 \% ) / BH 2 : BD 1 ( 20 , 98 \% : 2 \% ) / ET 3 ( 5 ) / ET 8 : Liq ( 25 , 50 \% : 50 \% ) / Liq ( 1 ) / Al ( 80 )$$

The numerals in parentheses represent a film thickness (unit: nm).

The numerals (97%:3%) represented by percentage in the same parentheses indicate a ratio (mass %) between the compound HT3 and the compound HA2 in the hole injecting layer, the numerals (98%:2%) represented by percentage in the same parentheses indicate a ratio (mass %) between the host material (compound BH1-75 or BH2) and the dopant material (compound BD1) in the first emitting layer or the second emitting layer, and the numerals (50%:50%) represented by percentage in the same parentheses indicate a ratio (mass %) between the compound ET8 and the compound Liq in the electron transporting layer (ET). Similar notations apply to the description below.

### Example 97

The organic EL device of Example 97 was manufactured in the same manner as that of Example 96 except that the compound BH1-75 (first host material) in the first emitting layer was replaced with the first compound shown in Table 20.

### Comparative Example 78

The organic EL device of Comparative Example 78 was manufactured in the same manner as that of Example 96 except that a 25-nm-thick first emitting layer was formed as the emitting layer, the first electron transporting layer was formed on the first emitting layer without forming the second emitting layer, and the first compound (first host material) in the first emitting layer was replaced with the first compound shown in Table 20.

### Comparative Example 79

The organic EL device of Comparative Example 79 was manufactured in the same manner as that of Example 96 except that a 25-nm-thick second emitting layer was formed on the second hole transporting layer without forming the first emitting layer as shown in Table 20.

TABLE 20

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Example 96 | BH1-75 | BD1 | 5 | BH2 | BD1 | 20 | 9.2 | 169 |
| Example 97 | R-BH3 | BD1 | 5 | BH2 | BD1 | 20 | — | 118 |
| Comparative | BH1-75 | BD1 | 25 | — | — | — | 6.0 | 63 |
| Ex. 78 | | | | | | | | |
| Comparative | — | — | — | BH2 | BD1 | 25 | 8.1 | 91 |
| Ex. 79 | | | | | | | | |

### Manufacture 6 of Organic EL Device

Organic EL devices were manufactured and evaluated as follows.

### Example 98

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HT5 and the compound HA2 were co-deposited on a surface provided with the transparent electrode line to cover the transparent electrode, thereby forming a 10-nm-thick hole injecting layer (HI). The ratios of the compound HT5 and the compound HA2 in the hole injecting layer were 97 mass % and 3 mass %, respectively.

After the formation of the hole injecting layer, the compound HT5 was vapor-deposited to form an 85-nm-thick first hole transporting layer (HT).

After the formation of the first hole transporting layer, the compound HT4 was vapor-deposited to form a 5-nm-thick second hole transporting layer (also referred to as an electron blocking layer (EBL)).

A compound BH1-64 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET3 was vapor-deposited on the second emitting layer to form a 5-nm-thick first electron transporting layer (also referred to as a hole blocking layer (HBL)).

The compound ET8 and the compound Liq were co-deposited on the first electron transporting layer (HBL) to form a 25-nm-thick electron transporting layer (ET). The ratios of the compound ET8 and the compound Liq in the electron transporting layer (ET) were 50 mass % and 50 mass %, respectively.

Liq was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 98 is roughly shown as follows.

$$ITO ( 130 ) / HT 5 : HA 2 ( 10 , 97 \% : 3 \% ) / HT 5 ( 85 ) / HT 4 ( 5 ) / BH 1 - 64 : BD 1 ( 5 , 98 \% : 2 \% ) / BH 2 : BD 1 ( 20 , 98 \% : 2 \% ) / ET 3 ( 5 ) / ET 8 : Liq ( 25 , 50 \% : 50 \% ) / Liq ( 1 ) / Al ( 80 )$$

The numerals in parentheses represent a film thickness (unit: nm).

The numerals (97%:3%) represented by percentage in the same parentheses indicate a ratio (mass %) between the compound HT5 and the compound HA2 in the hole injecting layer, the numerals (98%:2%) represented by percentage in the same parentheses indicate a ratio (mass %) between the host material (compound BH1-64 or BH2) and the dopant material (compound BD1) in the first emitting layer or the second emitting layer, and the numerals (50%:50%) represented by percentage in the same parentheses indicate a ratio (mass %) between the compound ET8 and the compound Liq in the electron transporting layer (ET). Similar notations apply to the description below.

### Examples 99 to 103

The organic EL devices of Examples 99 to 103 were manufactured in the same manner as that of Example 98 except that the compound BH1-6 (first host material) in the first emitting layer was replaced with the first compounds shown in Table 21.

### Comparative Examples 80 to 84

The organic EL devices of Comparative Examples 80 to 84 were manufactured in the same manner as that of Example 98 except that a 25-nm-thick first emitting layer was formed as the emitting layer, the first electron transporting layer was formed on the first emitting layer without forming the second emitting layer, and the first compound (first host material) in the first emitting layer was replaced with the first compounds listed in Table 21.

### Comparative Example 85

The organic EL device according to Comparative Example 85 was manufactured in the same manner as that of Example 98 except that a 25-nm-thick second emitting layer was formed on the second hole transporting layer without forming the first emitting layer as shown in Table 21.

TABLE 21

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Example 98 | BH1-64 | BD1 | 5 | BH2 | BD1 | 20 | 9.6 | 106 |
| Example 99 | BH1-65 | BD1 | 5 | BH2 | BD1 | 20 | 9.7 | 112 |
| Example 100 | BH1-66 | BD1 | 5 | BH2 | BD1 | 20 | 9.5 | 83 |
| Example 101 | BH1-67 | BD1 | 5 | BH2 | BD1 | 20 | 9.4 | 93 |
| Example 102 | BH1-68 | BD1 | 5 | BH2 | BD1 | 20 | 9.5 | 101 |
| Example 103 | R-BH3 | BD1 | 5 | BH2 | BD1 | 20 | 9.1 | — |
| Comparative | BH1-64 | BD1 | 25 | — | — | — | 6.1 | 31 |
| Ex. 80 | | | | | | | | |
| Comparative | BH1-65 | BD1 | 25 | — | — | — | 6.3 | 48 |
| Ex. 81 | | | | | | | | |
| Comparative | BH1-66 | BD1 | 25 | — | — | — | 6.1 | 31 |
| Ex. 82 | | | | | | | | |
| Comparative | BH1-67 | BD1 | 25 | — | — | — | 6.3 | 55 |
| Ex. 83 | | | | | | | | |
| Comparative | BH1-68 | BD1 | 25 | — | — | — | 6.0 | 28 |
| Ex. 84 | | | | | | | | |
| Comparative | — | — | — | BH2 | BD1 | 25 | 8.6 | 61 |
| Ex. 85 | | | | | | | | |

### Manufacture 7 of Organic EL Device

Organic EL devices were manufactured and evaluated as follows.

### Example 104

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HT5 and the compound HA2 were co-deposited on a surface provided with the transparent electrode line to cover the transparent electrode, thereby forming a 10-nm-thick hole injecting layer (HI). The ratios of the compound HT5 and the compound HA2 in the hole injecting layer were 97 mass % and 3 mass %, respectively.

After the formation of the hole injecting layer, the compound HT5 was vapor-deposited to form an 85-nm-thick first hole transporting layer (HT).

After the formation of the first hole transporting layer, the compound HT4 was vapor-deposited to form a 5-nm-thick second hole transporting layer (also referred to as an electron blocking layer (EBL)).

A compound BH1-70 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET1 was vapor-deposited on the second emitting layer to form a 5-nm-thick first electron transporting layer (also referred to as a hole blocking layer (HBL)).

The compound ET6 and the compound Liq were co-deposited on the first electron transporting layer (HBL) to form a 25-nm-thick electron transporting layer (ET). The ratios of the compound ET6 and the compound Liq in the electron transporting layer (ET) were 50 mass % and 50 mass %, respectively.

Liq was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 104 is roughly shown as follows.

$$ITO ( 130 ) / HT 5 : HA 2 ( 10 , 97 \% : 3 \% ) / HT 5 ( 85 ) / HT 4 ( 5 ) / BH 1 - 70 : BD 1 ( 5 , 98 \% : 2 \% ) / BH 2 : BD 1 ( 20 , 98 \% : 2 \% ) / ET 1 ( 5 ) / ET 6 : Liq ( 25 , 50 \% : 50 \% ) / Liq ( 1 ) / Al ( 80 )$$

The numerals in parentheses represent a film thickness (unit: nm).

The numerals (97%:3%) represented by percentage in the same parentheses indicate a ratio (mass %) between the compound HT5 and the compound HA2 in the hole injecting layer, the numerals (98%:2%) represented by percentage in the same parentheses indicate a ratio (mass %) between the host material (compound BH1-70 or BH2) and the dopant material (compound BD1) in the first emitting layer or the second emitting layer, and the numerals (50%:50%) represented by percentage in the same parentheses indicate a ratio (mass %) between the compound ET6 and the compound Liq in the electron transporting layer (ET). Similar notations apply to the description below.

### Examples 105 to 109

The organic EL devices of Examples 105 to 109 were manufactured in the same manner as that of Example 104 except that the compound BH1-70 (first host material) in the first emitting layer was replaced with the first compounds shown in in Table 70.

### Comparative Examples 86 to 90

The organic EL devices of Comparative Examples 86 to 90 were manufactured in the same manner as that of Example 104 except that a 25-nm-thick first emitting layer was formed as the emitting layer, the first electron transporting layer was formed on the first emitting layer without forming the second emitting layer, and the first compound (first host material) in the first emitting layer was replaced with the first compounds listed in Table 22.

### Comparative Example 91

The organic EL device of Comparative Example 91 was manufactured in the same manner as that of Example 104 except that a 25-nm-thick second emitting layer was formed on the second hole transporting layer without forming the first emitting layer shown in Table 104.

TABLE 22

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Example 104 | BH1-70 | BD1 | 5 | BH2 | BD1 | 20 | 10.2 | 185 |
| Example 105 | BH1-71 | BD1 | 5 | BH2 | BD1 | 20 | 10.7 | 223 |
| Example 106 | BH1-72 | BD1 | 5 | BH2 | BD1 | 20 | 10.4 | 212 |
| Example 107 | BH1-73 | BD1 | 5 | BH2 | BD1 | 20 | 10.6 | 220 |
| Example 108 | BH1-74 | BD1 | 5 | BH2 | BD1 | 20 | 10.3 | 218 |
| Example 109 | R-BH3 | BD1 | 5 | BH2 | BD1 | 20 | 8.7 | 101 |
| Comparative | BH1-70 | BD1 | 25 | — | — | — | 6.2 | 59 |
| Ex. 86 | | | | | | | | |
| Comparative | BH1-71 | BD1 | 25 | — | — | — | 6.6 | 63 |
| Ex. 87 | | | | | | | | |
| Comparative | BH1-72 | BD1 | 25 | — | — | — | 6.5 | 51 |
| Ex. 88 | | | | | | | | |
| Comparative | BH1-73 | BD1 | 25 | — | — | — | 6.5 | 62 |
| Ex. 89 | | | | | | | | |
| Comparative | BH1-74 | BD1 | 25 | — | — | — | 6.4 | 60 |
| Ex. 90 | | | | | | | | |
| Comparative | — | — | — | BH2 | BD1 | 25 | 8.3 | 76 |
| Ex. 91 | | | | | | | | |

### Manufacture 8 of Organic EL Device

### Example 110

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT1 was vapor-deposited to form an 80-nm-thick first hole transporting layer (HT).

After the formation of the first hole transporting layer, a compound HT8 was vapor-deposited to form a 10-nm-thick second hole transporting layer (also referred to as an electron blocking layer (EBL)).

A compound BH1-81 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET1 was vapor-deposited on the second emitting layer to form a 10-nm-thick first electron transporting layer (also referred to as a hole blocking layer (HBL)).

The compound ET2 was vapor-deposited on the first electron transporting layer to form a 15-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 110 is roughly shown as follows.

$$ITO ( 130 ) / HA 1 ( 5 ) / HT 1 ( 80 ) / HT 8 ( 10 ) / BH 1 - 81 : BD 1 ( 5 , 98 \% : 2 \% ) / BH 2 : BD 1 ( 20 , 98 \% : 2 \% ) / ET 1 ( 10 ) / ET 2 ( 15 ) / LiF ( 1 ) / Al ( 80 )$$

The numerals in parentheses represent a film thickness (unit: nm).

The numerals (98%:2%) represented by percentage in the same parentheses indicate a ratio (mass %) between the host material (compound BH1-81 or BH2) and the compound BD1 in the first emitting layer or the second emitting layer. Similar notations apply to the description below.

### Example 111

The organic EL device of Example 111 was manufactured in the same manner as that of Example 110 except that the compound BH1-81 (first host material) in the first emitting layer was replaced with the first compound shown in Table 23.

### Comparative Example 92

The organic EL device of Comparative Example 92 was manufactured in the same manner as that of Example 110 except that a 25-nm-thick first emitting layer was formed as the emitting layer and the first electron transporting layer was formed on the first emitting layer without forming the second emitting layer.

### Comparative Example 93

The organic EL device of Comparative Example 93 was manufactured in the same manner as that of Example 110 except that a 25-nm-thick second emitting layer was formed on the second hole transporting layer without forming the first emitting layer as shown in Table 23.

TABLE 23

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Example 110 | BH1-81 | BD1 | 5 | BH2 | BD1 | 20 | 10.7 | 134 |
| Example 111 | R-BH3 | BD1 | 5 | BH2 | BD1 | 20 | 10.4 | — |
| Comparative | BH1-81 | BD1 | 25 | — | — | — | 6.4 | 35 |
| Ex. 92 | | | | | | | | |
| Comparative | — | — | — | BH2 | BD1 | 25 | 10.2 | 102 |
| Ex. 93 | | | | | | | | |

### Manufacture 9 of Organic EL Device

### Examples 112 and 113

The organic EL devices of Examples 112 and 113 were manufactured in the same manner as that of Example 1 except that the compound BH1 (first host material) in the first emitting layer was replaced with the compounds shown in Table 24.

### Comparative Example 94

The organic EL device of Comparative Example 94 was manufactured in the same manner as that of Comparative Example 1 except that the compound BH1 (first host material) in the first emitting layer was replaced with the compound shown in Table 24.

TABLE 24

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Example 112 | BH1-82 | BD1 | 5 | BH2 | BD1 | 20 | 10.4 | 219 |
| Example 113 | R-BH3 | BD1 | 5 | BH2 | BD1 | 20 | 10.1 | 183 |
| Comparative | BH1-82 | BD1 | 25 | — | — | — | 6.2 | 71 |
| Ex. 94 | | | | | | | | |
| Comparative | — | — | — | BH2 | BD1 | 25 | 9.9 | 167 |
| Ex. 2 | | | | | | | | |

### Manufacture 10 of Organic EL Device

### Example 114

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT1 was vapor-deposited to form an 80-nm-thick first hole transporting layer (HT).

After the formation of the first hole transporting layer, the compound HT2 was vapor-deposited to form a 10-nm-thick second hole transporting layer (also referred to as an electron blocking layer (EBL)).

A compound BH1-83 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET7 was vapor-deposited on the second emitting layer to form a 10-nm-thick first electron transporting layer (also referred to as a hole blocking layer (HBL)).

The compound ET2 was vapor-deposited on the first electron transporting layer to form a 15-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 114 is roughly shown as follows.

$$ITO ( 130 ) / HA 1 ( 5 ) / HT 1 ( 80 ) / HT 2 ( 10 ) / BH 1 - 83 : BD 1 ( 5 , 98 \% : 2 \% ) / BH 2 : BD 1 ( 20 , 98 \% : 2 \% ) / ET 7 ( 10 ) / ET 2 ( 15 ) / LiF ( 1 ) / Al ( 80 )$$

The numerals in parentheses represent a film thickness (unit: nm).

The numerals (98%:2%) represented by percentage in the same parentheses indicate a ratio (mass %) between the host material (compound BH1-83 or BH2) and the compound BD1 in the first emitting layer or the second emitting layer. Similar notations apply to the description below.

### Example 115

The organic EL device of Example 115 was manufactured in the same manner as that of Example 114 except that the compound BH1-83 (first host material) in the first emitting layer was replaced with the first compound shown in Table 25.

### Comparative Example 95

The organic EL device of Comparative Example 95 was manufactured in the same manner as that of Example 114 except that a 25-nm-thick first emitting layer was formed as the emitting layer and the first electron transporting layer was formed on the first emitting layer without forming the second emitting layer.

### Comparative Example 96

The organic EL device of Comparative Example 96 was manufactured in the same manner as that of Example 114 except that a 25-nm-thick second emitting layer was formed on the second hole transporting layer without forming the first emitting layer as shown in Table 25.

TABLE 25

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Example 114 | BH1-83 | BD1 | 5 | BH2 | BD1 | 20 | 9.7 | 247 |
| Example 115 | R-BH3 | BD1 | 5 | BH2 | BD1 | 20 | 8.5 | — |
| Comparative | BH1-83 | BD1 | 25 | — | — | — | 6.0 | 76 |
| Ex. 95 | | | | | | | | |
| Comparative | — | — | — | BH2 | BD1 | 25 | 9.1 | 183 |
| Ex. 96 | | | | | | | | |

### Manufacture 11 of Organic EL Device

### Example 116

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT1 was vapor-deposited to form an 80-nm-thick first hole transporting layer (HT).

After the formation of the first hole transporting layer, the compound HT4 was vapor-deposited to form a 10-nm-thick second hole transporting layer (also referred to as an electron blocking layer (EBL)).

The compound BH1 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2-8 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET1 was vapor-deposited on the second emitting layer to form a 10-nm-thick first electron transporting layer (also referred to as a hole blocking layer (HBL)).

The compound ET2 was vapor-deposited on the first electron transporting layer to form a 20-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 116 is roughly shown as follows.

$$ITO ( 130 ) / HA 1 ( 5 ) / HT 1 ( 80 ) / HT 4 ( 10 ) / BH 1 : BD 1 ( 5 , 98 \% : 2 \% ) / BH 2 - 8 : BD 1 ( 20 , 98 \% : 2 \% ) / ET 1 ( 10 ) / ET 2 ( 20 ) / LiF ( 1 ) / Al ( 80 )$$

The numerals in parentheses represent a film thickness (unit: nm).

The numerals (98%:2%) represented by percentage in the same parentheses indicate a ratio (mass %) between the host material (compound BH1) and the compound BD1 in the first emitting layer, and numerals (98%:2%) represented by percentage in the same parentheses indicate a ratio (mass %) between the host material (compound BH2-8) and the compound BD1 in the second emitting layer. Similar notations apply to the description below.

### Example 117

The organic EL device of Example 117 was manufactured in the same manner as that of Example 116 except that the compound BH2-8 (second host material) in the second emitting layer was replaced with the second compound shown in Table 26.

### Comparative Example 97

The organic EL device of Comparative Example 97 was manufactured in the same manner as that of Example 116 except that a 25-nm-thick second emitting layer was formed on the second hole transporting layer without forming the first emitting layer, and the second compound (second host material) in the second emitting layer was replaced with the second compound shown in Table 26, as shown in Table 26.

TABLE 26

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 116 | BH1 | BD1 | 5 | BH2-8 | BD1 | 20 | 3.4 | 9.8 | 120 |
| Example 117 | BH1 | BD1 | 5 | BH2-5 | BD1 | 20 | 3.6 | 10.1 | 160 |
| Comparative | — | — | — | BH2-5 | BD1 | 25 | 3.8 | 8.9 | 110 |
| Ex. 97 | | | | | | | | | |

### Manufacture 12 of Organic EL Device

### Examples 118 and 119

The organic EL devices of Examples 118 and 119 were manufactured in the same manner as that of Example 116 except that the compound BH2-8 (second host material) in the second emitting layer was replaced with the second compounds shown in Table 27.

### Comparative Example 98

The organic EL device of Comparative Example 98 was manufactured in the same manner as that of Example 116 except that a 25-nm-thick second emitting layer was formed on the second hole transporting layer without forming the first emitting layer, and the second compound (second host material) in the second emitting layer was replaced with the second compound shown in Table 27.

TABLE 27

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 118 | BH1 | BD1 | 5 | BH2-2 | BD1 | 20 | 3.8 | 10.5 | 200 |
| Example 119 | BH1 | BD1 | 5 | BH2-10 | BD1 | 20 | 3.8 | 10.5 | 240 |
| Comparative | — | — | — | BH2-10 | BD1 | 25 | 4.0 | 9.8 | 140 |
| Ex. 98 | | | | | | | | | |

### Example 120

The organic EL device of Example 120 was manufactured in the same manner as that of Example 116 except that the compound BH2-8 (second host material) in the second emitting layer was replaced with the second compound shown in Table 28.

### Comparative Example 99

The organic EL device of Comparative Example 99 was manufactured in the same manner as that of Example 116 except that a 25-nm-thick second emitting layer was formed on the second hole transporting layer without forming the first emitting layer, and the second compound (second host material) in the second emitting layer was replaced with the second compound shown in Table 28, as shown in Table 28.

TABLE 28

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 116 | BH1 | BD1 | 5 | BH2-8 | BD1 | 20 | 3.4 | 9.8 | 120 |
| Example 120 | BH1 | BD1 | 5 | BH2-11 | BD1 | 20 | 3.4 | 9.8 | 150 |
| Comparative | — | — | — | BH2-11 | BD1 | 25 | 3.6 | 7.5 | 100 |
| Ex. 99 | | | | | | | | | |

### Manufacture 13 of Organic EL Device

### Example 121

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT3 was vapor-deposited to form an 80-nm-thick first hole transporting layer (HT).

After the formation of the first hole transporting layer, the compound HT4 was vapor-deposited to form a 10-nm-thick second hole transporting layer (also referred to as an electron blocking layer (EBL)).

The compound BH1 (first host material (BH)) and a compound BD2 (dopant material (BD)) were co-deposited on the second hole transporting layer such that the ratio of the compound BD2 accounted for 2 mass %, thereby forming a 5-nm-thick first emitting layer.

The compound BH2-2 (second host material (BH)) and the compound BD2 (dopant material (BD)) were co-deposited on the first emitting layer such that the ratio of the compound BD2 accounted for 2 mass %, thereby forming a 20-nm-thick second emitting layer.

The compound ET7 was vapor-deposited on the second emitting layer to form a 10-nm-thick first electron transporting layer (also referred to as a hole blocking layer (HBL)).

The compound ET2 was vapor-deposited on the first electron transporting layer to form a 20-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 121 is roughly shown as follows.

$$ITO ( 130 ) / HA 1 ( 5 ) / HT 3 ( 80 ) / HT 4 ( 10 ) / BH 1 : BD 2 ( 5 , 98 \% : 2 \% ) / BH 2 - 2 : BD 2 ( 20 , 98 \% : 2 \% ) / ET 7 ( 10 ) / ET 2 ( 20 ) / LiF ( 1 ) / Al ( 80 )$$

The numerals in parentheses represent a film thickness (unit: nm).

The numerals (98%:2%) represented by percentage in the same parentheses indicate a ratio (mass %) between the host material (compound BH1) and the compound BD2 in the first emitting layer, and numerals (98%:2%) represented by percentage in the same parentheses indicate a ratio (mass %) between the host material (compound BH2-2) and the compound BD2 in the second emitting layer. Similar notations apply to the description below.

### Example 122

The organic EL device of Example 122 was manufactured in the same manner as that of Example 121 except that the compound BH2-2 (second host material) in the second emitting layer was replaced with the second compound shown in Table 29.

### Comparative Example 100

The organic EL device of Comparative Example 100 was manufactured in the same manner as that of Example 121 except that a 25-nm-thick second emitting layer was formed on the second hole transporting layer without forming the first emitting layer, and the second compound (second host material) in the second emitting layer was replaced with the second compound shown in Table 29, as shown in Table 29.

TABLE 29

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 121 | BH1 | BD2 | 5 | BH2-2 | BD2 | 20 | 3.8 | 10.1 | 180 |
| Example 122 | BH1 | BD2 | 5 | BH2-12 | BD2 | 20 | 4.0 | 10.3 | 200 |
| Comparative | — | — | — | BH2-12 | BD2 | 25 | 4.2 | 8.8 | 110 |
| Ex. 100 | | | | | | | | | |

### Manufacture 14 of Organic EL Device

### Example 123

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT5 was vapor-deposited to form an 80-nm-thick first hole transporting layer (HT).

After the formation of the first hole transporting layer, a compound HT6 was vapor-deposited to form a 10-nm-thick second hole transporting layer (also referred to as an electron blocking layer (EBL)).

A compound BH1-10 (first host material (BH)) and the compound BD2 (dopant material (BD)) were co-deposited on the second hole transporting layer such that the ratio of the compound BD2 accounted for 2 mass %, thereby forming a 5-nm-thick first emitting layer.

The compound BH2-2 (second host material (BH)) and the compound BD2 (dopant material (BD)) were co-deposited on the first emitting layer such that the ratio of the compound BD2 accounted for 2 mass %, thereby forming a 20-nm-thick second emitting layer.

The compound ET7 was vapor-deposited on the second emitting layer to form a 10-nm-thick first electron transporting layer (also referred to as a hole blocking layer (HBL)).

The compound ET2 was vapor-deposited on the first electron transporting layer to form a 20-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 123 is roughly shown as follows.

$$ITO ( 130 ) / HA 1 ( 5 ) / HT 5 ( 80 ) / HT 6 ( 10 ) / BH 1 - 10 : BD 2 ( 5 , 98 \% : 2 \% ) / BH 2 - 2 : BD 2 ( 20 , 98 \% : 2 \% ) / ET 7 ( 10 ) / ET 2 ( 20 ) / LiF ( 1 ) / Al ( 80 )$$

The numerals in parentheses represent a film thickness (unit: nm).

The numerals (98%:2%) represented by percentage in the same parentheses indicate a ratio (mass %) between the host material (compound BH1-10) and the compound BD2 in the first emitting layer, and numerals (98%:2%) represented by percentage in the same parentheses indicate a ratio (mass %) between the host material (compound BH2-2) and the compound BD2 in the second emitting layer. Similar notations apply to the description below.

### Example 124

The organic EL device of Example 124 was manufactured in the same manner as that of Example 123 except that the compound BH2-2 (second host material) in the second emitting layer was replaced with the second compound shown in Table 30.

### Comparative Example 101

The organic EL device of Comparative Example 101 was manufactured in the same manner as that of Example 123 except that a 25-nm-thick second emitting layer was formed on the second hole transporting layer without forming the first emitting layer, and the second compound (second host material) in the second emitting layer was replaced with the second compound shown in Table 30, as shown in Table 30.

TABLE 30

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 123 | BH1-10 | BD2 | 5 | BH2-2 | BD2 | 20 | 3.9 | 10.0 | 210 |
| Example 124 | BH1-10 | BD2 | 5 | BH2-13 | BD2 | 20 | 3.8 | 10.3 | 190 |
| Comparative | — | — | — | BH2-13 | BD2 | 25 | 4.1 | 9.2 | 110 |
| Ex. 101 | | | | | | | | | |

### Manufacture 15 of Organic EL Device

### Example 125

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT3 was vapor-deposited to form an 80-nm-thick first hole transporting layer (HT).

After the formation of the first hole transporting layer, the compound HT7 was vapor-deposited to form a 10-nm-thick second hole transporting layer (also referred to as an electron blocking layer (EBL)).

The compound BH1-10 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2-2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET7 was vapor-deposited on the second emitting layer to form a 10-nm-thick first electron transporting layer (also referred to as a hole blocking layer (HBL)).

The compound ET2 was vapor-deposited on the first electron transporting layer to form a 20-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 125 is roughly shown as follows.

$$ITO ( 130 ) / HA 1 ( 5 ) / HT 3 ( 80 ) / HT 7 ( 10 ) / BH 1 - 10 : BD 1 ( 5 , 98 \% : 2 \% ) / BH 2 - 8 : BD 1 ( 20 , 98 \% : 2 \% ) / ET 7 ( 10 ) / ET 2 ( 20 ) / LiF ( 1 ) / Al ( 80 )$$

The numerals in parentheses represent a film thickness (unit: nm).

The numerals (98%:2%) represented by percentage in the same parentheses indicate a ratio (mass %) between the host material (compound BH1-10) and the compound BD1 in the first emitting layer, and numerals (98%:2%) represented by percentage in the same parentheses indicate a ratio (mass %) between the host material (compound BH2-2) and the compound BD1 in the second emitting layer. Similar notations apply to the description below.

### Example 126

The organic EL device of Example 126 was manufactured in the same manner as that of Example 125 except that the compound BH2-2 (second host material) in the second emitting layer was replaced with the second compound shown in Table 31.

### Comparative Example 102

The organic EL device of Comparative Example 102 was manufactured in the same manner as that of Example 125 except that a 25-nm-thick second emitting layer was formed on the second hole transporting layer without forming the first emitting layer, and the second compound (second host material) in the second emitting layer was replaced with the second compound shown in Table 31, as shown in Table 31.

TABLE 31

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 125 | BH1-10 | BD1 | 5 | BH2-2 | BD1 | 20 | 4.0 | 10.5 | 150 |
| Example 126 | BH1-10 | BD1 | 5 | BH2-14 | BD1 | 20 | 4.0 | 10.8 | 160 |
| Comparative | — | — | — | BH2-14 | BD1 | 25 | 4.2 | 9.5 | 100 |
| Ex. 102 | | | | | | | | | |

### Example 127

The organic EL device of Example 127 was manufactured in the same manner as that of Example 125 except that the compound BH2-2 (second host material) in the second emitting layer was replaced with the second compound shown in Table 32.

### Comparative Example 103

The organic EL device of Comparative Example 103 was manufactured in the same manner as that of Example 125 except that a 25-nm-thick second emitting layer was formed on the second hole transporting layer without forming the first emitting layer, and the second compound (second host material) in the second emitting layer was replaced with the second compound shown in Table 32, as shown in Table 32.

TABLE 32

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 125 | BH1-10 | BD1 | 5 | BH2-2 | BD1 | 20 | 4.0 | 10.5 | 150 |
| Example 127 | BH1-10 | BD1 | 5 | BH2-15 | BD1 | 20 | 3.9 | 10.3 | 180 |
| Comparative | — | — | — | BH2-15 | BD1 | 25 | 4.0 | 9.2 | 80 |
| Ex. 103 | | | | | | | | | |

### Example 128

The organic EL device of Example 128 was manufactured in the same manner as that of Example 125 except that the compound BH2-2 (second host material) in the second emitting layer was replaced with the second compound shown in Table 33.

### Comparative Example 104

The organic EL device of Comparative Example 104 was manufactured in the same manner as that of Example 125 except that a 25-nm-thick second emitting layer was formed on the second hole transporting layer without forming the first emitting layer, and the second compound (second host material) in the second emitting layer was replaced with the second compound shown in Table 33, as shown in Table 33.

TABLE 33

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 125 | BH1-10 | BD1 | 5 | BH2-2 | BD1 | 20 | 4.0 | 10.5 | 150 |
| Example 128 | BH1-10 | BD1 | 5 | BH2-16 | BD1 | 20 | 3.8 | 10.5 | 170 |
| Comparative | — | — | — | BH2-16 | BD1 | 25 | 4.1 | 9.5 | 70 |
| Ex. 104 | | | | | | | | | |

### Example 129

The organic EL device of Example 129 was manufactured in the same manner as that of Example 125 except that the compound BH2-2 (second host material) in the second emitting layer was replaced with the second compound shown in Table 34.

### Comparative Example 105

The organic EL device of Comparative Example 105 was manufactured in the same manner as that of Example 125 except that a 25-nm-thick second emitting layer was formed on the second hole transporting layer without forming the first emitting layer, and the second compound (second host material) in the second emitting layer was replaced with the second compound shown in Table 34, as shown in Table 34.

TABLE 34

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 125 | BH1-10 | BD1 | 5 | BH2-2 | BD1 | 20 | 4.0 | 10.5 | 150 |
| Example 129 | BH1-10 | BD1 | 5 | BH2-17 | BD1 | 20 | 3.7 | 10.6 | 170 |
| Comparative | — | — | — | BH2-17 | BD1 | 25 | 4.0 | 9.1 | 60 |
| Ex. 105 | | | | | | | | | |

### Manufacture 16 of Organic EL Device

### Example 130

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT3 was vapor-deposited to form an 80-nm-thick first hole transporting layer (HT).

After the formation of the first hole transporting layer, the compound HT7 was vapor-deposited to form a 10-nm-thick second hole transporting layer (also referred to as an electron blocking layer (EBL)).

The compound BH1-10 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2-8 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET1 was vapor-deposited on the second emitting layer to form a 10-nm-thick first electron transporting layer (also referred to as a hole blocking layer (HBL)).

The compound ET5 was vapor-deposited on the first electron transporting layer to form a 20-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 130 is roughly shown as follows.

$$ITO ( 130 ) / HA 1 ( 5 ) / HT 3 ( 80 ) / HT 7 ( 10 ) / BH 1 - 10 : BD 1 ( 5 , 98 \% : 2 \% ) / BH 2 - 8 : BD 1 ( 20 , 98 \% : 2 \% ) / ET 1 ( 10 ) / ET 5 ( 20 ) / LiF ( 1 ) / Al ( 80 )$$

The numerals in parentheses represent a film thickness (unit: nm).

The numerals (98%:2%) represented by percentage in the same parentheses indicate a ratio (mass %) between the host material (compound BH1-10) and the compound BD1 in the first emitting layer, and numerals (98%:2%) represented by percentage in the same parentheses indicate a ratio (mass %) between the host material (compound BH2-8) and the compound BD1 in the second emitting layer. Similar notations apply to the description below.

### Example 131

The organic EL device of Example 131 was manufactured in the same manner as that of Example 130 except that the compound BH2-8 (second host material) in the second emitting layer was replaced with the second compound shown in Table 35.

### Comparative Example 106

The organic EL device of Comparative Example 106 was manufactured in the same manner as that of Example 130 except that a 25-nm-thick second emitting layer was formed on the second hole transporting layer without forming the first emitting layer, and the second compound (second host material) in the second emitting layer was replaced with the second compound shown in Table 35, as shown in Table 35.

TABLE 35

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 130 | BH1-10 | BD1 | 5 | BH2-8 | BD1 | 20 | 3.4 | 9.5 | 140 |
| Example 131 | BH1-10 | BD1 | 5 | BH2-18 | BD1 | 20 | 3.4 | 10.0 | 150 |
| Comparative | — | — | — | BH2-18 | BD1 | 25 | 3.6 | 9.0 | 100 |
| Ex. 106 | | | | | | | | | |

### Example 132

The organic EL device of Example 132 was manufactured in the same manner as that of Example 130 except that the compound BH2-8 (second host material) in the second emitting layer was replaced with the second compound shown in Table 36.

### Comparative Example 107

The organic EL device of Comparative Example 107 was manufactured in the same manner as that of Example 130 except that a 25-nm-thick second emitting layer was formed on the second hole transporting layer without forming the first emitting layer, and the second compound (second host material) in the second emitting layer was replaced with the second compound shown in Table 36, as shown in Table 36.

TABLE 36

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 130 | BH1-10 | BD1 | 5 | BH2-8 | BD1 | 20 | 3.4 | 9.5 | 140 |
| Example 132 | BH1-10 | BD1 | 5 | BH2-19 | BD1 | 20 | 3.5 | 10.3 | 140 |
| Comparative | — | — | — | BH2-19 | BD1 | 25 | 3.6 | 9.2 | 80 |
| Ex. 107 | | | | | | | | | |

### Example 133

The organic EL device of Example 133 was manufactured in the same manner as that of Example 130 except that the compound BH2-8 (second host material) in the second emitting layer was replaced with the second compound shown in Table 37.

### Comparative Example 108

The organic EL device of Comparative Example 108 was manufactured in the same manner as that of Example 130 except that a 25-nm-thick second emitting layer was formed on the second hole transporting layer without forming the first emitting layer, and the second compound (second host material) in the second emitting layer was replaced with the second compound shown in Table 37, as shown in Table 37.

TABLE 37

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 130 | BH1-10 | BD1 | 5 | BH2-8 | BD1 | 20 | 3.4 | 9.5 | 140 |
| Example 133 | BH1-10 | BD1 | 5 | BH2-20 | BD1 | 20 | 3.4 | 9.9 | 160 |
| Comparative | — | — | — | BH2-20 | BD1 | 25 | 3.7 | 8.8 | 120 |
| Ex. 108 | | | | | | | | | |

### Manufacture 17 of Organic EL Device

### Example 134

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT1 was vapor-deposited to form an 80-nm-thick first hole transporting layer (HT).

After the formation of the first hole transporting layer, the compound HT2 was vapor-deposited to form a 10-nm-thick second hole transporting layer (also referred to as an electron blocking layer (EBL)).

The compound BH1 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2-8 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET4 was vapor-deposited on the second emitting layer to form a 10-nm-thick first electron transporting layer (also referred to as a hole blocking layer (HBL)).

The compound ET2 was vapor-deposited on the first electron transporting layer to form a 20-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 134 is roughly shown as follows.

$$ITO ( 130 ) / HA 1 ( 5 ) / HT 1 ( 80 ) / HT 2 ( 10 ) / BH 1 : BD 1 ( 5 , 98 \% : 2 \% ) / BH 2 - 8 : BD 1 ( 20 , 98 \% : 2 \% ) / ET 4 ( 10 ) / ET 2 ( 20 ) / LiF ( 1 ) / Al ( 80 )$$

The numerals in parentheses represent a film thickness (unit: nm).

The numerals (98%:2%) represented by percentage in the same parentheses indicate a ratio (mass %) between the host material (compound BH1) and the compound BD1 in the first emitting layer, and numerals (98%:2%) represented by percentage in the same parentheses indicate a ratio (mass %) between the host material (compound BH2-8) and the compound BD1 in the second emitting layer. Similar notations apply to the description below.

### Example 135

The organic EL device of Example 135 was manufactured in the same manner as that of Example 134 except that the compound BH2-8 (second host material) in the second emitting layer was replaced with the second compound shown in Table 38.

### Comparative Example 109

The organic EL device of Comparative Example 109 was manufactured in the same manner as that of Example 134 except that a 25-nm-thick second emitting layer was formed on the second hole transporting layer without forming the first emitting layer, and the second compound (second host material) in the second emitting layer was replaced with the second compound shown in Table 38, as shown in Table 38.

TABLE 38

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 134 | BH1 | BD1 | 5 | BH2-8 | BD1 | 20 | 3.3 | 9.8 | 90 |
| Example 135 | BH1 | BD1 | 5 | BH2-21 | BD1 | 20 | 3.3 | 9.6 | 130 |
| Comparative | — | — | — | BH2-21 | BD1 | 25 | 3.5 | 8.5 | 80 |
| Ex. 109 | | | | | | | | | |

### Example 136

The organic EL device of Example 136 was manufactured in the same manner as that of Example 134 except that the compound BH2-8 (second host material) in the second emitting layer was replaced with the second compound shown in Table 39.

### Comparative Example 110

The organic EL device of Comparative Example 110 was manufactured in the same manner as that of Example 134 except that a 25-nm-thick second emitting layer was formed on the second hole transporting layer without forming the first emitting layer, and the second compound (second host material) in the second emitting layer was replaced with the second compound shown in Table 39, as shown in Table 39.

TABLE 39

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 134 | BH1 | BD1 | 5 | BH2-8 | BD1 | 20 | 3.3 | 9.8 | 90 |
| Example 136 | BH1 | BD1 | 5 | BH2-22 | BD1 | 20 | 3.4 | 8.3 | 140 |
| Comparative | — | — | — | BH2-22 | BD1 | 25 | 3.5 | 7.3 | 80 |
| Ex. 110 | | | | | | | | | |

### Example 137

The organic EL device of Example 137 was manufactured in the same manner as that of Example 134 except that the compound BH2-8 (second host material) in the second emitting layer was replaced with the second compound shown in Table 40.

### Comparative Example 111

The organic EL device of Comparative Example 111 was manufactured in the same manner as that of Example 134 except that a 25-nm-thick second emitting layer was formed on the second hole transporting layer without forming the first emitting layer, and the second compound (second host material) in the second emitting layer was replaced with the second compound shown in Table 40, as shown in Table 40.

TABLE 40

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 134 | BH1 | BD1 | 5 | BH2-8 | BD1 | 20 | 3.3 | 9.8 | 90 |
| Example 137 | BH1 | BD1 | 5 | BH2-23 | BD1 | 20 | 3.3 | 8.8 | 130 |
| Comparative | — | — | — | BH2-23 | BD1 | 25 | 3.4 | 8.0 | 80 |
| Ex. 111 | | | | | | | | | |

### Example 138

The organic EL device of Example 138 was manufactured in the same manner as that of Example 134 except that the compound BH2-8 (second host material) in the second emitting layer was replaced with the second compound shown in Table 41.

### Comparative Example 112

The organic EL device of Comparative Example 112 was manufactured in the same manner as that of Example 134 except that a 25-nm-thick second emitting layer was formed on the second hole transporting layer without forming the first emitting layer, and the second compound (second host material) in the second emitting layer was replaced with the second compound shown in Table 41, as shown in Table 41.

TABLE 41

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 134 | BH1 | BD1 | 5 | BH2-8 | BD1 | 20 | 3.3 | 9.8 | 90 |
| Example 138 | BH1 | BD1 | 5 | BH2-24 | BD1 | 20 | 3.5 | 9.1 | 120 |
| Comparative | — | — | — | BH2-24 | BD1 | 25 | 3.7 | 7.8 | 90 |
| Ex. 112 | | | | | | | | | |

### Example 139

The organic EL device of Example 139 was manufactured in the same manner as that of Example 134 except that the compound BH2-8 (second host material) in the second emitting layer was replaced with the second compound shown in Table 42.

### Comparative Example 113

The organic EL device of Comparative Example 113 was manufactured in the same manner as that of Example 134 except that a 25-nm-thick second emitting layer was formed on the second hole transporting layer without forming the first emitting layer, and the second compound (second host material) in the second emitting layer was replaced with the second compound shown in Table 42, as shown in Table 42.

TABLE 42

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 134 | BH1 | BD1 | 5 | BH2-8 | BD1 | 20 | 3.3 | 9.8 | 90 |
| Example 139 | BH1 | BD1 | 5 | BH2-25 | BD1 | 20 | 3.4 | 9.4 | 130 |
| Comparative | — | — | — | BH2-25 | BD1 | 25 | 3.4 | 7.1 | 70 |
| Ex. 113 | | | | | | | | | |

### Example 140

The organic EL device of Example 140 was manufactured in the same manner as that of Example 143 except that the compound BH2-8 (second host material) in the second emitting layer was replaced with the second compound shown in Table 43.

### Comparative Example 114

The organic EL device of Comparative Example 114 was manufactured in the same manner as that of Example 134 except that a 25-nm-thick second emitting layer was formed on the second hole transporting layer without forming the first emitting layer, and the second compound (second host material) in the second emitting layer was replaced with the second compound shown in Table 43, as shown in Table 43.

TABLE 43

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 134 | BH1 | BD1 | 5 | BH2-8 | BD1 | 20 | 3.3 | 9.8 | 90 |
| Example 140 | BH1 | BD1 | 5 | BH2-26 | BD1 | 20 | 3.5 | 9.2 | 130 |
| Comparative | — | — | — | BH2-26 | BD1 | 25 | 3.4 | 7.5 | 75 |
| Ex. 114 | | | | | | | | | |

### Example 141

The organic EL device of Example 141 was manufactured in the same manner as that of Example 134 except that the compound BH2-8 (second host material) in the second emitting layer was replaced with the second compound shown in Table 44.

### Comparative Example 115

The organic EL device of Comparative Example 115 was manufactured in the same manner as that of Example 134 except that a 25-nm-thick second emitting layer was formed on the second hole transporting layer without forming the first emitting layer, and the second compound (second host material) in the second emitting layer was replaced with the second compound shown in Table 44, as shown in Table 44.

TABLE 44

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 134 | BH1 | BD1 | 5 | BH2-8 | BD1 | 20 | 3.3 | 9.8 | 90 |
| Example 141 | BH1 | BD1 | 5 | BH2-27 | BD1 | 20 | 3.2 | 9.1 | 130 |
| Comparative | — | — | — | BH2-27 | BD1 | 25 | 3.5 | 7.2 | 80 |
| Ex. 115 | | | | | | | | | |

### Example 142

The organic EL device of Example 142 was manufactured in the same manner as that of Example 134 except that the compound BH2-8 (second host material) in the second emitting layer was replaced with the second compound shown in Table 45.

### Comparative Example 116

The organic EL device of Comparative Example 116 was manufactured in the same manner as that of Example 134 except that a 25-nm-thick second emitting layer was formed on the second hole transporting layer without forming the first emitting layer, and the second compound (second host material) in the second emitting layer was replaced with the second compound shown in Table 45, as shown in Table 45.

TABLE 45

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 134 | BH1 | BD1 | 5 | BH2-8 | BD1 | 20 | 3.3 | 9.8 | 90 |
| Example 142 | BH1 | BD1 | 5 | BH2-28 | BD1 | 20 | 3.3 | 9.0 | 140 |
| Comparative | — | — | — | BH2-28 | BD1 | 25 | 3.4 | 7.4 | 65 |
| Ex. 116 | | | | | | | | | |

### Manufacture 18 of Organic EL Device

### Example 143

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT1 was vapor-deposited to form an 80-nm-thick first hole transporting layer (HT).

After the formation of the first hole transporting layer, the compound HT2 was vapor-deposited to form a 10-nm-thick second hole transporting layer (also referred to as an electron blocking layer (EBL)).

The compound BH1 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2-8 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET7 was vapor-deposited on the second emitting layer to form a 10-nm-thick first electron transporting layer (also referred to as a hole blocking layer (HBL)).

The compound ET2 was vapor-deposited on the first electron transporting layer to form a 20-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 143 is roughly shown as follows.

$$ITO ( 130 ) / HA 1 ( 5 ) / HT 1 ( 80 ) / HT 2 ( 10 ) / BH 1 : BD 1 ( 5 , 98 \% : 2 \% ) / BH 2 - 8 : BD 1 ( 20 , 98 \% : 2 \% ) / ET 7 ( 10 ) / ET 2 ( 20 ) / LiF ( 1 ) / Al ( 80 )$$

The numerals in parentheses represent a film thickness (unit: nm).

The numerals (98%:2%) represented by percentage in the same parentheses indicate a ratio (mass %) between the host material (compound BH1) and the compound BD1 in the first emitting layer, and numerals (98%:2%) represented by percentage in the same parentheses indicate a ratio (mass %) between the host material (compound BH2-8) and the compound BD1 in the second emitting layer. Similar notations apply to the description below.

### Example 144

The organic EL device of Example 144 was manufactured in the same manner as that of Example 143 except that the compound BH2-8 (second host material) in the second emitting layer was replaced with the second compound shown in Table 46.

### Comparative Example 117

The organic EL device of Comparative Example 117 was manufactured in the same manner as that of Example 143 except that a 25-nm-thick second emitting layer was formed on the second hole transporting layer without forming the first emitting layer, and the second compound (second host material) in the second emitting layer was replaced with the second compound shown in Table 46, as shown in Table 46.

TABLE 46

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Example 143 | BH1 | BD1 | 5 | BH2-8 | BD1 | 20 | 3.5 | 9.0 | 120 |
| Example 144 | BH1 | BD1 | 5 | BH2-29 | BD1 | 20 | 4.0 | 10.1 | 80 |
| Comparative | — | — | — | BH2-29 | BD1 | 25 | 4.5 | 8.2 | 40 |
| Ex. 117 | | | | | | | | | |

### Example 145

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT1 was vapor-deposited to form an 80-nm-thick first hole transporting layer (HT).

After the formation of the first hole transporting layer, the compound HT2 was vapor-deposited to form a 10-nm-thick second hole transporting layer (also referred to as an electron blocking layer (EBL)).

A compound 1BH-1 (first host material (BH)) and the compound BD1 (dopant material (BD)) were co-deposited on the second hole transporting layer so that a ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET1 was vapor-deposited on the second emitting layer to form a 10-nm-thick first electron transporting layer (also referred to as a hole blocking layer (HBL)).

The compound ET2 was vapor-deposited on the first electron transporting layer to form a 15-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 145 is roughly shown as follows.

$$ITO ( 130 ) / HA 1 ( 5 ) / HT 1 ( 80 ) / HT 2 ( 10 ) / 1 BH - 1 : BD 1 ( 5 , 98 \% : 2 \% ) / BH 2 : BD 1 ( 20 , 98 \% : 2 \% ) / ET 1 ( 10 ) / ET 2 ( 15 ) / LiF ( 1 ) / Al ( 80 )$$

The numerals in parentheses represent a film thickness (unit: nm).

The numerals (98%:2%) represented by percentage in the same parentheses indicate a ratio (mass %) between the host material (compound 1BH-1 or compound BH2) and the compound BD1 in the first emitting layer or the second emitting layer.

### Comparative Example 118

The organic EL device of Comparative Example 118 was manufactured in the same manner as that of Example 145 except that a 25-nm-thick first emitting layer was formed as the emitting layer and the first electron transporting layer was formed on the first emitting layer without forming the second emitting layer, as shown in Table 47.

TABLE 47

| | First Emitting Layer | | | Second Emitting Layer | | | |
|---|---|---|---|---|---|---|---|
| | | | Film | | | Film | |
| | First | Third | Thickness | Second | Fourth | Thickness | EQE |
| | Compound | Compound | [nm] | Compound | Compound | [nm] | [%] |
| Example 145 | 1BH-1 | BD1 | 5 | BH2 | BD1 | 20 | 10.5 |
| Comparative | 1BH-1 | BD1 | 25 | — | — | — | 7.5 |
| Ex. 118 | | | | | | | |
| Comparative | — | — | — | BH2 | BD1 | 25 | 9.9 |
| Ex. 2 | | | | | | | |

### Manufacture 19 of Organic EL Device

Organic EL devices were manufactured and evaluated as follows.

### Example 146

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT1 was vapor-deposited to form an 80-nm-thick first hole transporting layer (HT).

After the formation of the first hole transporting layer, the compound HT2 was vapor-deposited to form a 10-nm-thick second hole transporting layer (also referred to as an electron blocking layer (EBL)).

A compound BH3-1 (first host material (BH)) and the compound BD2 (dopant material (BD)) were co-deposited on the second hole transporting layer so that a ratio of the compound BD2 accounted for 2 mass %, thereby forming a 5-nm-thick first emitting layer.

A compound BH3-2 (second host material (BH)) and the compound BD2 (dopant material (BD)) were co-deposited on the first emitting layer so that a ratio of the compound BD2 accounted for 2 mass %, thereby forming a 20-nm-thick second emitting layer.

The compound ET1 was vapor-deposited on the second emitting layer to form a 10-nm-thick first electron transporting layer (also referred to as a hole blocking layer (HBL)).

The compound ET2 was vapor-deposited on the first electron transporting layer to form a 15-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 146 is roughly shown as follows.

$$ITO ( 130 ) / HA 1 ( 5 ) / HT 1 ( 80 ) / HT 2 ( 10 ) / BH 3 - 1 : BD 2 ( 5 , 98 \% : 2 \% ) / BH 3 - 2 : BD 2 ( 20 , 98 \% : 2 \% ) / ET 1 ( 10 ) / ET 2 ( 15 ) / LiF ( 1 ) / Al ( 80 )$$

The numerals in parentheses represent a film thickness (unit: nm).

The numerals (98%:2%) represented by percentage in the same parentheses indicate a ratio (mass %) between the host material (compound BH3-1 or compound BH3-2) and the compound BD2 in the first emitting layer or the second emitting layer.

### Comparative Example 119

The organic EL device of Comparative Example 119 was manufactured in the same manner as that of Example 146 except that a 25-nm-thick first emitting layer was formed as the emitting layer and the first electron transporting layer was formed on the first emitting layer without forming the second emitting layer, as shown in Table 48.

### Comparative Example 120

The organic EL device of Comparative Example 120 was manufactured in the same manner as that of Example 146 except that a 25-nm-thick second emitting layer was formed as the emitting layer on the second hole transporting layer without forming the first emitting layer as shown in Table 48.

TABLE 48

| | First Emitting Layer | | | | Second Emitting Layer | | | | | | |
|---|---|---|---|---|---|---|---|---|---|---|---|
| | First | | | | Second | | | | | | |
| | Compound | | Third | Film | Compound | | Fourth | Film | Drive | | |
| | | T₁ | Compound | Thickness | | T₁ | Compound | Thickness | Voltage | EQE | LT95 |
| | Name | [ev] | Name | [nm] | Name | [ev] | Name | [nm] | [V] | [%] | [h] |
| Example 146 | BH3-1 | 2.11 | BD2 | 5 | BH3-2 | 2.08 | BD2 | 20 | 3.4 | 9.5 | 200 |
| Comparative | BH3-1 | 2.11 | BD2 | 25 | — | — | — | — | 3.8 | 8.2 | 92 |
| Ex. 119 | | | | | | | | | | | |
| Comparative | — | — | — | — | BH3-2 | 2.08 | BD2 | 25 | 3.4 | 7.1 | 125 |
| Ex. 120 | | | | | | | | | | | |

### Example 147

The organic EL device of Example 147 was manufactured in the same manner as that of Example 146 except that the compound BH3-1 (first host material) in the first emitting layer was replaced with the first compound shown in Table 49.

### Comparative Example 121

The organic EL device of Comparative Example 121 was manufactured in the same manner as that of Comparative Example 119 except that the compound BH3-1 (first host material) in the first emitting layer was replaced with the first compound shown in Table 49.

TABLE 49

| | First Emitting Layer | | | | Second Emitting Layer | | | | | | |
|---|---|---|---|---|---|---|---|---|---|---|---|
| | First | | | | Second | | | | | | |
| | Compound | | Third | Film | Compound | | Fourth | Film | Drive | | |
| | | T₁ | Compound | Thickness | | T₁ | Compound | Thickness | Voltage | EQE | LT95 |
| | Name | [ev] | Name | [nm] | Name | [ev] | Name | [nm] | [V] | [%] | [h] |
| Example 147 | BH3-3 | 2.23 | BD2 | 5 | BH3-2 | 2.08 | BD2 | 20 | 3.5 | 9.1 | 230 |
| Comparative | BH3-3 | 2.23 | BD2 | 25 | — | — | — | — | 3.6 | 6.8 | 52 |
| Ex. 121 | | | | | | | | | | | |
| Comparative | — | — | — | — | BH3-2 | 2.08 | BD2 | 25 | 3.4 | 7.1 | 125 |
| Ex. 120 | | | | | | | | | | | |

### Example 148

The organic EL device of Example 148 was manufactured in the same manner as that of Example 146 except that the compound BH3-1 (first host material) in the first emitting layer and the compound BH3-2 in the second emitting layer were respectively replaced with the first compound and the second compound shown in Table 50.

TABLE 50

| | First Emitting Layer | | | | Second Emitting Layer | | | | | |
|---|---|---|---|---|---|---|---|---|---|---|
| | First | | | | Second | | | | | |
| | Compound | | Third | Film | Compound | | Fourth | Film | Drive | |
| | | T₁ | Compound | Thickness | | T₁ | Compound | Thickness | Voltage | EQE |
| | Name | [ev] | Name | [nm] | Name | [ev] | Name | [nm] | [V] | [%] |
| Example 148 | BH3-3 | 2.23 | BD2 | 5 | BH3-1 | 2.11 | BD2 | 20 | 3.3 | 10.1 |
| Comparative | BH3-3 | 2.23 | BD2 | 25 | — | — | — | — | 3.6 | 6.8 |
| Ex. 121 | | | | | | | | | | |
| Comparative | BH3-1 | 2.11 | BD2 | 25 | — | — | — | — | 3.8 | 8.2 |
| Ex. 119 | | | | | | | | | | |

### Example 149

The organic EL device of Example 149 was manufactured in the same manner as that of Example 146 except that the compound BH3-1 (first host material) in the first emitting layer and the compound BH3-2 in the second emitting layer were respectively replaced with the first compound and the second compound shown in Table 51.

### Comparative Example 122

The organic EL device of Comparative Example 122 was manufactured in the same manner as that of Comparative Example 119 except that the compound BH3-1 (first host material) in the first emitting layer was replaced with the first compound shown in Table 51.

### Comparative Example 123

The organic EL device of Comparative Example 123 was manufactured in the same manner as that of Comparative Example 120 except that the compound BH3-2 (first host material) in the second emitting layer was replaced with the second compound shown in Table 51.

TABLE 51

| | First Emitting Layer | | | | Second Emitting Layer | | | | | | |
|---|---|---|---|---|---|---|---|---|---|---|---|
| | First | | | | Second | | | | | | |
| | Compound | | Third | Film | Compound | | Fourth | Film | Drive | | |
| | | T₁ | Compound | Thickness | | T₁ | Compound | Thickness | Voltage | EQE | LT95 |
| | Name | [ev] | Name | [nm] | Name | [ev] | Name | [nm] | [V] | [%] | [h] |
| Example 149 | BH3-4 | 2.27 | BD2 | 5 | BH3-5 | 1.80 | BD2 | 20 | 3.2 | 10.5 | 165 |
| Comparative | BH3-4 | 2.27 | BD2 | 25 | — | — | — | — | 3.4 | 6.2 | 103 |
| Ex. 122 | | | | | | | | | | | |
| Comparative | — | — | — | — | BH3-5 | 1.80 | BD2 | 25 | 4.0 | 8.8 | 140 |
| Ex. 123 | | | | | | | | | | | |

### Example 150

The organic EL device of Example 150 was manufactured in the same manner as that of Example 146 except that the compound BH3-1 (first host material) in the first emitting layer was replaced with the first compound shown in Table 52.

TABLE 52

| | First Emitting Layer | | | | Second Emitting Layer | | | | | |
|---|---|---|---|---|---|---|---|---|---|---|
| | First | | | | Second | | | | | |
| | Compound | | Third | Film | Compound | | Fourth | Film | Drive | |
| | | T₁ | Compound | Thickness | | T₁ | Compound | Thickness | Voltage | EQE |
| | Name | [ev] | Name | [nm] | Name | [ev] | Name | [nm] | [V] | [%] |
| Example 150 | BH3-4 | 2.27 | BD2 | 5 | BH3-2 | 2.08 | BD2 | 20 | 3.4 | 9.5 |
| Comparative | BH3-4 | 2.27 | BD2 | 25 | — | — | — | — | 3.4 | 6.2 |
| Ex. 122 | | | | | | | | | | |
| Comparative | — | — | — | — | BH3-2 | 2.08 | BD2 | 25 | 3.4 | 7.1 |
| Ex. 120 | | | | | | | | | | |

### Example 151

The organic EL device of Example 151 was manufactured in the same manner as that of Example 146 except that the compound BH3-1 (first host material) in the first emitting layer and the compound BH3-2 in the second emitting layer were respectively replaced with the first compound and the second compound shown in Table 53.

TABLE 53

| | First Emitting Layer | | | | Second Emitting Layer | | | | | | |
|---|---|---|---|---|---|---|---|---|---|---|---|
| | First | | | | Second | | | | | | |
| | Compound | | Third | Film | Compound | | Fourth | Film | Drive | | |
| | | T₁ | Compound | Thickness | | T₁ | Compound | Thickness | Voltage | EQE | LT95 |
| | Name | [ev] | Name | [nm] | Name | [ev] | Name | [nm] | [V] | [%] | [h] |
| Example 151 | BH3-4 | 2.27 | BD2 | 5 | BH3-1 | 2.11 | BD2 | 20 | 3.3 | 9.9 | 180 |
| Comparative | BH3-4 | 2.27 | BD2 | 25 | — | — | — | — | 3.4 | 6.2 | 103 |
| Ex. 122 | | | | | | | | | | | |
| Comparative | BH3-1 | 2.11 | BD2 | 25 | — | — | — | — | 3.8 | 8.2 | 92 |
| Ex. 119 | | | | | | | | | | | |

### Example 152

The organic EL device of Example 152 was manufactured in the same manner as that of Example 146 except that the compound BH3-1 (first host material) in the first emitting layer and the compound BH3-2 in the second emitting layer were respectively replaced with the first compound and the second compound shown in Table 54.

### Comparative Example 124

The organic EL device of Comparative Example 124 was manufactured in the same manner as that of Comparative Example 119 except that the compound BH3-1 (first host material) in the first emitting layer was replaced with the first compound shown in Table 54.

TABLE 54

| | First Emitting Layer | | | | Second Emitting Layer | | | | | | |
|---|---|---|---|---|---|---|---|---|---|---|---|
| | First | | | | Second | | | | | | |
| | Compound | | Third | Film | Compound | | Fourth | Film | Drive | | |
| | | T₁ | Compound | Thickness | | T₁ | Compound | Thickness | Voltage | EQE | LT95 |
| | Name | [ev] | Name | [nm] | Name | [ev] | Name | [nm] | [V] | [%] | [h] |
| Example 152 | BH3-6 | 2.35 | BD2 | 5 | BH3-5 | 1.80 | BD2 | 20 | 3.5 | 10.0 | 175 |
| Comparative | BH3-6 | 2.35 | BD2 | 25 | — | — | — | — | 4.2 | 4.5 | 21 |
| Ex. 124 | | | | | | | | | | | |
| Comparative | — | — | — | — | BH3-5 | 1.80 | BD2 | 25 | 4.0 | 8.8 | 140 |
| Ex. 123 | | | | | | | | | | | |

### Example 153

The organic EL device of Example 153 was manufactured in the same manner as that of Example 146 except that the compound BH3-1 (first host material) in the first emitting layer was replaced with the first compound shown in Table 55.

TABLE 55

| | First Emitting Layer | | | | Second Emitting Layer | | | | | | |
|---|---|---|---|---|---|---|---|---|---|---|---|
| | First | | | | Second | | | | | | |
| | Compound | | Third | Film | Compound | | Fourth | Film | Drive | | |
| | | T₁ | Compound | Thickness | | T₁ | Compound | Thickness | Voltage | EQE | LT95 |
| | Name | [ev] | Name | [nm] | Name | [ev] | Name | [nm] | [V] | [%] | [h] |
| Example 153 | BH3-6 | 2.35 | BD2 | 5 | BH3-2 | 2.08 | BD2 | 20 | 3.6 | 9.2 | 210 |
| Comparative | BH3-6 | 2.35 | BD2 | 25 | — | — | — | — | 4.2 | 4.5 | 21 |
| Ex. 124 | | | | | | | | | | | |
| Comparative | — | — | — | — | BH3-2 | 2.08 | BD2 | 25 | 3.4 | 7.1 | 125 |
| Ex. 120 | | | | | | | | | | | |

### Example 154

The organic EL device of Example 154 was manufactured in the same manner as that of Example 146 except that the compound BH3-1 (first host material) in the first emitting layer and the compound BH3-2 in the second emitting layer were respectively replaced with the first compound and the second compound shown in Table 56.

TABLE 56

| | First Emitting Layer | | | | Second Emitting Layer | | | | | | |
|---|---|---|---|---|---|---|---|---|---|---|---|
| | First | | | | Second | | | | | | |
| | Compound | | Third | Film | Compound | | Fourth | Film | Drive | | |
| | | T₁ | Compound | Thickness | | T₁ | Compound | Thickness | Voltage | EQE | LT95 |
| | Name | [ev] | Name | [nm] | Name | [ev] | Name | [nm] | [V] | [%] | [h] |
| Example 154 | BH3-6 | 2.35 | BD2 | 5 | BH3-1 | 2.11 | BD2 | 20 | 3.6 | 9.8 | 190 |
| Comparative | BH3-6 | 2.35 | BD2 | 25 | — | — | — | — | 4.2 | 4.5 | 21 |
| Ex. 124 | | | | | | | | | | | |
| Comparative | BH3-1 | 2.11 | BD2 | 25 | — | — | — | — | 3.8 | 8.2 | 92 |
| Ex. 119 | | | | | | | | | | | |

### Example 155

The organic EL device of Example 155 was manufactured in the same manner as that of Example 146 except that the compound BH3-1 (first host material) in the first emitting layer and the compound BH3-2 in the second emitting layer were respectively replaced with the first compound and the second compound shown in Table 57.

### Comparative Example 125

The organic EL device of Comparative Example 125 was manufactured in the same manner as that of Comparative Example 120 except that the compound BH3-2 (second host material) in the second emitting layer was replaced with the second compound shown in Table 57.

TABLE 57

| | First Emitting Layer | | | | Second Emitting Layer | | | | | | | | |
|---|---|---|---|---|---|---|---|---|---|---|---|---|---|
| | First | | | | Second | | | | | | | | |
| | Compound | | Third | Film | Compound | | Fourth | Film | Drive | | | | |
| | | T₁ | Compound | Thickness | | T₁ | Compound | Thickness | Voltage | | | λp | EQE |
| | Name | [ev] | Name | [nm] | Name | [ev] | Name | [nm] | [V] | CIE-x | CIE-y | [nm] | [%] |
| Example 155 | BH3-7 | 2.20 | BD1 | 5 | BH2 | 1.87 | BD1 | 20 | 3.48 | 0.135 | 0.104 | 460 | 10.4 |
| Comparative | — | — | — | — | BH2 | 1.87 | BD1 | 25 | 3.61 | 0.134 | 0.104 | 460 | 9.9 |
| Ex. 125 | | | | | | | | | | | | | |

### Manufacture 20 of Organic EL Device

Organic EL devices were manufactured and evaluated as follows.

### Example 156

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HT1 and the compound HA2 were co-deposited on a surface provided with the transparent electrode line to cover the transparent electrode, thereby forming a 5-nm-thick hole injecting layer (HI). The ratios of the compound HT1 and the compound HA2 in the hole injecting layer were 97 mass % and 3 mass %, respectively.

After the formation of the hole injecting layer, the compound HT1 was vapor-deposited to form an 80-nm-thick first hole transporting layer (HT).

Subsequent to the formation of the first hole transporting layer, a compound HT9 was vapor-deposited to form a 10-nm-thick second hole transporting layer (also referred to as an electron blocking layer (EBL)).

A compound BH1-84 (first host material (BH)) and the compound BD2 (dopant material (BD)) were co-deposited on the second hole transporting layer such that the ratio of the compound BD2 accounted for 2 mass %, thereby forming a 5-nm-thick first emitting layer.

A compound BH2-3 (second host material (BH)) and the compound BD2 (dopant material (BD)) were co-deposited on the first emitting layer so that a ratio of the compound BD2 accounted for 2 mass %, thereby forming a 20-nm-thick second emitting layer.

The compound ET7 was vapor-deposited on the second emitting layer to form a 10-nm-thick first electron transporting layer (also referred to as a hole blocking layer (HBL)).

The compound ET2 was vapor-deposited on the first electron transporting layer to form a 15-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Example 156 is roughly shown as follows.

$$ITO ( 130 ) / HT 1 : HA 2 ( 5 , 97 \% : 3 \% ) / HT 1 ( 80 ) / HT 9 ( 10 ) / BH 1 - 84 : BD 2 ( 5 , 98 \% : 2 \% ) / BH 2 - 3 : BD 2 ( 20 , 98 \% : 2 \% ) / ET 7 ( 10 ) / ET 2 ( 15 ) / LiF ( 1 ) / Al ( 80 )$$

The numerals in parentheses represent a film thickness (unit: nm).

The numerals (97%:3%) represented by percentage in the same parentheses indicate a ratio (mass %) between the compound HT1 and the compound HA2 in the hole injecting layer. The numerals (98%:2%) represented by percentage in the same parentheses indicate a ratio (mass %) between the host material (compound BH1-84 or compound BH2-3) and the compound BD2 in the first emitting layer or the second emitting layer. Similar notations apply to the description below.

### Examples 157 to 160

The organic EL devices of Examples 157 and 158 were manufactured in the same manner as that of Example 156 except that the compound BH1-84 (first host material) in the first emitting layer was replaced with the second compounds shown in Table 58.

The organic EL device of Example 159 was manufactured in the same manner as that of Example 156 except that the compound BH2-3 (second host material) in the second emitting layer was replaced with the second compound shown in Table 58.

The organic EL device of Example 160 was manufactured in the same manner as that of Example 159 except that the compound BH1-84 (first host material) in the first emitting layer was replaced with the first compound shown in Table 58.

### Comparative Examples 126 to 128

The organic EL devices of Comparative Examples 126 to 128 were manufactured in the same manner as that of Example 156 except that a 25-nm-thick first emitting layer was formed as the emitting layer, the first electron transporting layer was formed on the first emitting layer without forming the second emitting layer, and the compound BH1-84 (first host material) in the first emitting layer was replaced with the first compound shown in Table 58.

### Comparative Example 129

The organic EL device of Comparative Example 129 was manufactured in the same manner as that of Example 156 except that a 25-nm-thick second emitting layer was formed as the emitting layer on the second hole transporting layer without forming the first emitting layer as shown in Table 58.

### Comparative Example 130

The organic EL device of Comparative Example 130 was manufactured in the same manner as that of Example 160 except that a 25-nm-thick first emitting layer was formed as the emitting layer, and the first electron transporting layer was formed on the first emitting layer without forming the second emitting layer as shown in Table 58.

### Comparative Example 131

The organic EL device of Comparative Example 131 was manufactured in the same manner as that of Example 160 except that a 25-nm-thick second emitting layer was formed as the emitting layer on the second hole transporting layer without forming the first emitting layer as shown in Table 58.

TABLE 58

| | First Emitting Layer | | | | | Second Emitting Layer | | | | | | |
|---|---|---|---|---|---|---|---|---|---|---|---|---|
| | First | | Third | | | Second | | Fourth | | | | |
| | Compound | | Compound | | Film | Compound | | Compound | | Film | | |
| | | T₁ | | T₁ | Thickness | | T₁ | | T₁ | Thickness | EQE | LT95 |
| | Name | [ev] | Name | [ev] | [nm] | Name | [ev] | Name | [ev] | [nm] | [%] | [hr] |
| Example 156 | BH1-84 | 2.09 | BD2 | 2.60 | 5 | BH2-3 | 1.87 | BD2 | 2.60 | 20 | 9.6 | 320 |
| Example 157 | BH1-85 | 2.09 | BD2 | 2.60 | 5 | BH2-3 | 1.87 | BD2 | 2.60 | 20 | 9.6 | 250 |
| Example 158 | BH1-86 | 2.09 | BD2 | 2.60 | 5 | BH2-3 | 1.87 | BD2 | 2.60 | 20 | 9.6 | 160 |
| Example 159 | BH1-84 | 2.09 | BD2 | 2.60 | 5 | BH2 | 1.87 | BD2 | 2.60 | 20 | 9.7 | 255 |
| Example 160 | BH1-87 | 2.09 | BD2 | 2.60 | 5 | BH2 | 1.87 | BD2 | 2.60 | 20 | 9.8 | 150 |
| Comparative | BH1-84 | 2.09 | BD2 | 2.60 | 25 | — | — | — | — | — | 7.2 | 80 |
| Ex. 126 | | | | | | | | | | | | |
| Comparative | BH1-85 | 2.09 | BD2 | 2.60 | 25 | — | — | — | — | — | 7.2 | 75 |
| Ex. 127 | | | | | | | | | | | | |
| Comparative | BH1-86 | 2.09 | BD2 | 2.60 | 25 | — | — | — | — | — | 7.2 | 75 |
| Ex. 128 | | | | | | | | | | | | |
| Comparative | — | — | — | — | — | BH2-3 | 1.87 | BD2 | 2.60 | 25 | 8.5 | 145 |
| Ex. 129 | | | | | | | | | | | | |
| Comparative | BH1-87 | 2.09 | BD2 | 2.60 | 25 | — | — | — | — | — | 7.3 | 75 |
| Ex. 130 | | | | | | | | | | | | |
| Comparative | — | — | — | — | — | BH2 | 1.87 | BD2 | 2.60 | 25 | 8.8 | 78 |
| Ex. 131 | | | | | | | | | | | | |

### Examples 161 to 166

The organic EL devices of Examples 161 to 166 were manufactured in the same manner as that of Example 156 except that at least one of the compound BH1-84 in the first emitting layer or the compound BH2-3 in the second emitting layer was replaced with the compounds shown in Table 59.

### Comparative Example 132

The organic EL device of Comparative Example 132 was manufactured in the same manner as that of Example 156 except that a 25-nm-thick first emitting layer was formed as the emitting layer, the first electron transporting layer was formed on the first emitting layer without forming the second emitting layer, and the compound BH1-84 in the first emitting layer was replaced with the first compound shown in Table 59, as shown in Table 59.

### Comparative Example 133

The organic EL device of Comparative Example 133 was manufactured in the same manner as that of Comparative Example 130 except that the compound BH1-87 in the first emitting layer was replaced with the first compound shown in Table 59, as shown in Table 59.

TABLE 59

| | First Emitting Layer | | | | | Second Emitting Layer | | | | | | |
|---|---|---|---|---|---|---|---|---|---|---|---|---|
| | First | | Third | | | Second | | Fourth | | | | |
| | Compound | | Compound | | Film | Compound | | Compound | | Film | | |
| | | T₁ | | T₁ | Thickness | | T₁ | | T₁ | Thickness | EQE | LT95 |
| | Name | [ev] | Name | [ev] | [nm] | Name | [ev] | Name | [ev] | [nm] | [%] | [hr] |
| Example 161 | BH4-3 | 2.10 | BD2 | 2.60 | 5 | BH2-3 | 1.87 | BD2 | 2.60 | 20 | 9.8 | 290 |
| Example 162 | BH4-3 | 2.10 | BD2 | 2.60 | 5 | BH2 | 1.87 | BD2 | 2.60 | 20 | 9.9 | 230 |
| Example 163 | BH4-3 | 2.10 | BD2 | 2.60 | 5 | BH1-84 | 2.09 | BD2 | 2.60 | 20 | 9.0 | 250 |
| Example 164 | BH4-3 | 2.10 | BD2 | 2.60 | 5 | BH1-87 | 2.09 | BD2 | 2.60 | 20 | 9.2 | 240 |
| Example 165 | BH4-4 | 2.10 | BD2 | 2.60 | 5 | BH2 | 1.87 | BD2 | 2.60 | 20 | 10.0 | 140 |
| Example 166 | BH4-4 | 2.10 | BD2 | 2.60 | 5 | BH1-87 | 2.09 | BD2 | 2.60 | 20 | 9.5 | 140 |
| Comparative | BH4-3 | 2.10 | BD2 | 2.60 | 25 | — | — | — | — | — | 8.4 | 110 |
| Ex. 132 | | | | | | | | | | | | |
| Comparative | BH1-84 | 2.09 | BD2 | 2.60 | 25 | — | — | — | — | — | 7.2 | 80 |
| Ex. 126 | | | | | | | | | | | | |
| Comparative | — | — | — | — | — | BH2-3 | 1.87 | BD2 | 2.60 | 25 | 8.5 | 145 |
| Ex. 129 | | | | | | | | | | | | |
| Comparative | BH4-4 | 2.10 | BD2 | 2.60 | 25 | — | — | — | — | — | 8.5 | 60 |
| Ex. 133 | | | | | | | | | | | | |
| Comparative | BH1-87 | 2.09 | BD2 | 2.60 | 25 | — | — | — | — | — | 7.3 | 75 |
| Ex. 130 | | | | | | | | | | | | |
| Comparative | — | — | — | — | — | BH2 | 1.87 | BD2 | 2.60 | 25 | 8.8 | 78 |
| Ex. 131 | | | | | | | | | | | | |

### Examples 167 to 172

The organic EL devices of Examples 167 to 172 were manufactured in the same manner as that of Example 156 except that at least one of the compound BH1-84 in the first emitting layer or the compound BH2-3 in the second emitting layer was replaced with the compound shown in Table 60.

### Comparative Examples 134 and 135

The organic EL devices of Comparative Example 134 and 135 were manufactured in the same manner as that of Example 156 except that a 25-nm-thick first emitting layer was formed as the emitting layer, the first electron transporting layer was formed on the first emitting layer without forming the second emitting layer, and the compound BH1-84 in the first emitting layer was replaced with the first compound, as shown in Table 60.

TABLE 60

| | First Emitting Layer | | | | | Second Emitting Layer | | | | | | |
|---|---|---|---|---|---|---|---|---|---|---|---|---|
| | First | | Third | | | Second | | Fourth | | | | |
| | Compound | | Compound | | Film | Compound | | Compound | | Film | | |
| | | T₁ | | T₁ | Thickness | | T₁ | | T₁ | Thickness | EQE | LT95 |
| | Name | [ev] | Name | [ev] | [nm] | Name | [ev] | Name | [ev] | [nm] | [%] | [hr] |
| Example 167 | BH4-1 | 2.25 | BD2 | 2.60 | 5 | BH2-3 | 1.87 | BD2 | 2.60 | 20 | 9.2 | 350 |
| Example 168 | BH4-1 | 2.25 | BD2 | 2.60 | 5 | BH2 | 1.87 | BD2 | 2.60 | 20 | 9.5 | 290 |
| Example 169 | BH4-1 | 2.25 | BD2 | 2.60 | 5 | BH4-3 | 2.10 | BD2 | 2.60 | 20 | 9.5 | 270 |
| Example 170 | BH4-1 | 2.25 | BD2 | 2.60 | 5 | BH4-4 | 2.10 | BD2 | 2.60 | 20 | 9.7 | 230 |
| Example 171 | BH4-2 | 2.25 | BD2 | 2.60 | 5 | BH2 | 1.87 | BD2 | 2.60 | 20 | 9.9 | 200 |
| Example 172 | BH4-2 | 2.25 | BD2 | 2.60 | 5 | BH4-4 | 2.10 | BD2 | 2.60 | 20 | 10.0 | 180 |
| Comparative | BH4-1 | 2.25 | BD2 | 2.60 | 25 | — | — | — | — | — | 8.0 | 157 |
| Ex. 134 | | | | | | | | | | | | |
| Comparative | BH4-3 | 2.10 | BD2 | 2.60 | 25 | — | — | — | — | — | 8.4 | 110 |
| Ex. 132 | | | | | | | | | | | | |
| Comparative | — | — | — | — | — | BH2-3 | 1.87 | BD2 | 2.60 | 25 | 8.5 | 145 |
| Ex. 129 | | | | | | | | | | | | |
| Comparative | BH4-2 | 2.25 | BD2 | 2.60 | 25 | — | — | — | — | — | 8.2 | 80 |
| Ex. 135 | | | | | | | | | | | | |
| Comparative | BH4-4 | 2.10 | BD2 | 2.60 | 25 | — | — | — | — | — | 8.5 | 60 |
| Ex. 133 | | | | | | | | | | | | |
| Comparative | — | — | — | — | — | BH2 | 1.87 | BD2 | 2.60 | 25 | 8.8 | 78 |
| Ex. 131 | | | | | | | | | | | | |

### Manufacture 21 of Organic EL Device

Organic EL devices were manufactured and evaluated as follows.

### Example 173

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, a compound HA3 was vapor-deposited on a surface provided with the transparent electrode line to cover the transparent electrode, thereby forming a 10-nm-thick hole injecting layer (HI).

After the formation of the hole injecting layer, a compound HT10 was vapor-deposited to form a 90-nm-thick first hole transporting layer (HT).

After the formation of the first hole transporting layer, a compound HT11 was vapor-deposited to form a 5-nm-thick second hole transporting layer (also referred to as an electron blocking layer (EBL)).

A compound BH4-5 (first host material (BH)) and the compound BD2 (dopant material (BD)) were co-deposited on the second hole transporting layer so that a ratio of the compound BD2 accounted for 2 mass %, thereby forming a 5-nm-thick first emitting layer.

A compound BH3-5 (second host material (BH)) and the compound BD2 (dopant material (BD)) were co-deposited on the first emitting layer so that a ratio of the compound BD2 accounted for 2 mass %, thereby forming a 15-nm-thick second emitting layer.

The compound ET9 was vapor-deposited on the second emitting layer to form a 5-nm-thick first electron transporting layer (also referred to as a hole blocking layer (HBL)).

The compound ET2 was vapor-deposited on the first electron transporting layer to form a 20-nm-thick second electron transporting layer (ET).

LiF was vapor-deposited on the second electron transporting layer to form a 1-nm-thick electron injecting layer.

Metal (A1) was vapor-deposited on the electron injecting layer to form a 50-nm-thick cathode.

A device arrangement of the organic EL device in Example 173 is roughly shown as follows.

$$ITO ( 130 ) / HA 3 ( 10 ) / HT 10 ( 90 ) / HT 11 ( 5 ) / BH 4 - 5 : BD 2 ( 5 , 98 \% : 2 \% ) / BH 3 - 5 : BD 2 ( 15 , 98 \% : 2 \% ) / ET 9 ( 5 ) / ET 2 ( 20 ) / LiF ( 1 ) / Al ( 50 )$$

The numerals in parentheses represent a film thickness (unit: nm).

The numerals (98%:2%) represented by percentage in the same parentheses indicate a ratio (mass %) between the host material (BH4-5) and the compound BD2 in the first emitting layer. The numerals (98%:2%) represented by percentage in the same parentheses indicate a ratio (mass %) between the host material (compound BH3-5) and the compound BD2 in the second emitting layer.

### Example 174

The organic EL device of Example 174 was manufactured in the same manner as that of Example 173 except that the compound BH4-5 (first host material) in the first emitting layer was replaced with the first compound shown in Table 61 and the compounds BD2 in the first emitting layer and the second emitting layer were respectively replaced with the third compound and the fourth compound shown in Table 61.

### Reference Example 1

An organic EL device of Reference Example 1 was manufactured in the same manner as that of Example 173 except that the compound BH4-5 (first host material) in the first emitting layer was replaced with the first compound shown in Table 61.

TABLE 61

| | First Emitting Layer | | | | | Second Emitting Layer | | | | | | |
|---|---|---|---|---|---|---|---|---|---|---|---|---|
| | First | | Third | | | Second | | Fourth | | | | |
| | Compound | | Compound | | Film | Compound | | Compound | | Film | | |
| | | T₁ | | T₁ | Thickness | | T₁ | | T₁ | Thickness | EQE | LT90 |
| | Name | [ev] | Name | [ev] | [nm] | Name | [ev] | Name | [ev] | [nm] | [%] | [hr] |
| Example 173 | BH4-5 | 2.02 | BD2 | 2.60 | 5 | BH3-5 | 1.80 | BD2 | 2.60 | 15 | 10.4 | 254 |
| Example 174 | BH1-10 | 2.09 | BD4 | 2.45 | 5 | BH3-5 | 1.80 | BD4 | 2.45 | 15 | 10.6 | 255 |
| Reference | BH1-10 | 2.09 | BD2 | 2.60 | 5 | BH3-5 | 1.80 | BD2 | 2.60 | 15 | 10.5 | 214 |
| Ex. 1 | | | | | | | | | | | | |

### Evaluation Method of Compounds

### Triplet Energy T₁

A measurement target compound was dissolved in EPA (diethylether:isopentane:ethanol=5:5:2 in volume ratio) at a concentration of 10 μmol/L, and the obtained solution was encapsulated in a quartz cell to provide a measurement sample. A phosphorescence spectrum (ordinate axis: phosphorescent luminous intensity, abscissa axis: wavelength) of the measurement sample was measured at a low temperature (77K). A tangent was drawn to the rise of the phosphorescence spectrum close to the short-wavelength region. An energy amount was calculated by a conversion equation (F1) below on a basis of a wavelength value λ_{edge} [nm] at an intersection of the tangent and the abscissa axis. The calculated energy amount was defined as triplet energy T₁. It should be noted that the triplet energy T₁ has an error of about plus or minus 0.02 eV depending on measurement conditions.

$${T}_{1} [ eV ] = 1239.85 / {λ}_{edge} \qquad Conversion Equation ( F1 )$$

The tangent to the rise of the phosphorescence spectrum close to the short-wavelength region is drawn as follows. While moving on a curve of the phosphorescence spectrum from the short-wavelength region to the local maximum spectral value closest to the short-wavelength region among the local maximum spectral values, a tangent is checked at each point on the curve toward the long-wavelength region of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 manufactured by Hitachi High-Technologies Corporation was used.

### Singlet Energy S₁

A toluene solution of a measurement target compound at a concentration of 10 μmol/L was prepared and put in a quartz cell. An absorption spectrum (ordinate axis: absorption intensity, abscissa axis: wavelength) of the thus-obtained sample was measured at a normal temperature (300K). A tangent was drawn to the fall of the absorption spectrum close to the long-wavelength region, and a wavelength value λedge (nm) at an intersection of the tangent and the abscissa axis was assigned to a conversion equation (F2) below to calculate the singlet energy.

$${S}_{1} [ eV ] = 1239 \text{.85} / {λ}_{edge} \qquad Conversion Equation ( F2 )$$

A spectrophotometer (U3310 manufactured by Hitachi, Ltd.) was used for measuring absorption spectrum.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve fell (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

Measurement values of a singlet energy S₁ and a triplet energy T₁ of each of the compounds BH1, BH2, BH2-3 and BH4 are shown in Tables.

A singlet energy S₁ of the compound BD1 was 2.73 eV.

A triplet energy T₁ of the compound BD1 was 2.29 eV.

Measurement values of other compounds are shown in Tables.

### Stokes Shift (SS) (nm)

A measurement target compound was dissolved in toluene at a concentration of 2.0×10⁻⁵ mol/L to prepare a measurement sample. The measurement sample was put into a quartz cell and was irradiated with continuous light falling within an ultraviolet-to-visible region at a room temperature (300K) to measure an absorption spectrum (ordinate axis: absorbance, abscissa axis: wavelength). A spectrophotometer U-3900/3900H manufactured by Hitachi High-Tech Science Corporation was used for the absorption spectrum measurement. Moreover, a measurement target compound was dissolved in toluene at a concentration of 4.9×10⁻⁶ mol/L to prepare a measurement sample. The measurement sample was put into a quartz cell and was irradiated with excited light at a room temperature (300K) to measure fluorescence spectrum (ordinate axis: fluorescence intensity, abscissa axis: wavelength). A spectrophotofluorometer F-7000 manufactured by Hitachi High-Tech Science Corporation was used for the fluorescence spectrum measurement.

A difference between an absorption local-maximum wavelength and a fluorescence local-maximum wavelength was calculated from the absorption spectrum and the fluorescence spectrum to obtain a Stokes shift (SS). A unit of the Stokes shift (SS) was denoted by nm.

A Stokes shift (SS) of the compound BD1 was 14 nm.

### Preparation of Toluene Solution

The compound BD1 was dissolved in toluene at a concentration of 4.9×10⁻⁶ mol/L to prepare a toluene solution of the compound BD1. A toluene solution of the compound BD2 and a toluene solution of the compound BD3 were prepared in the same manner.

### Measurement of Fluorescence Main Peak Wavelength (FL-Peak)

Fluorescence main peak wavelength of the toluene solution of the compound BD1 excited at 390 nm was measured using a fluorescence spectrometer (spectrophotofluorometer F-7000 (manufactured by Hitachi High-Tech Science Corporation)). The fluorescence main peak wavelengths of the toluene solutions of the compound BD2 and the compound BD3 were measured in the same manner as the compound BD1.

The fluorescence main peak wavelength of the compound BD1 was 453 nm.

The fluorescence main peak wavelength of the compound BD2 was 455 nm.

The fluorescence main peak wavelength of the compound BD3 was 451 nm.

1. An organic electroluminescence device comprising:
an anode;
a cathode;
a first emitting layer; and
a second emitting layer, wherein
the first emitting layer is provided between the anode and the cathode,
the second emitting layer is provided between the first emitting layer and the cathode,
the first emitting layer includes a first host material and a first dopant material,
the second emitting layer includes a second host material and a second dopant material,
the first host material and the second host material are mutually different,
the first dopant material is a compound that emits fluorescence having a main peak wavelength of 500 nm or less,
the second dopant material is a compound that emits fluorescence having a main peak wavelength of 500 nm or less,
the compound included in the first emitting layer and that emits fluorescence having a main peak wavelength of 500 nm or less is the same as or different from the compound included in the second emitting layer and that emits fluorescence having a main peak wavelength of 500 nm or less,
the first host material is a first compound represented by a formula (1X), formula (12X), formula (13X), formula (14X), formula (15X) or formula (16X) below,
a triplet energy T₁(H1) of the first host material and a triplet energy T₁(H2) of the second host material satisfy a relationship of a numerical formula (Numerical Formula 1) below, and
the triplet energy T₁(H1) of the first host material and a triplet energy T₁(D1) of the first dopant material satisfy a relationship of a numerical formula (Numerical Formula 2A) below, a triplet energy T₁(D2) of the second dopant material and the triplet energy T₁(H2) of the second host material satisfy a relationship of a numerical formula (Numerical Formula 3) below, or both the relationship of the numerical formula (Numerical Formula 2A) and the relationship of the numerical formula (Numerical Formula 3) are satisfied,
$${T}_{1} ( H 1 ) > {T}_{1} ( H 2 ) \qquad ( Numerical Formula 1 )$$
$${T}_{1} ( D 1 ) > {T}_{1} ( H 1 ) \qquad ( Numerical Formula 2 A )$$
$${T}_{1} ( D 2 ) > {T}_{1} ( H 2 ) \qquad ( Numerical Formula 3 )$$ 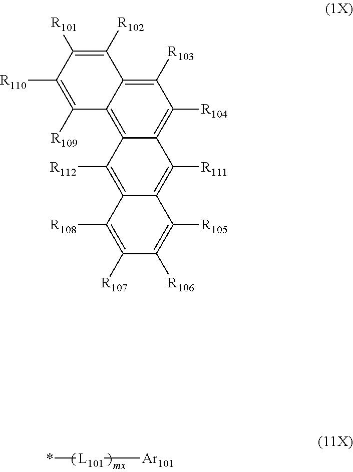
where, in the formula (1X):
R₁₀₁ to R₁₁₂ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11X);
at least one of R₁₀₁ to R₁₁₂ is a group represented by the formula (11X);
when a plurality of groups represented by the formula (11X) are present, the plurality of groups represented by the formula (11X) are mutually the same or different;
L₁₀₁ is a single bond, a substituted or unsubstituted arylene group having 6 to 50 ring carbon atoms, or a substituted or unsubstituted divalent heterocyclic group having 5 to 50 ring atoms;
Ar₁₀₁ is a substituted or unsubstituted aryl group having 6 to 50 ring carbon atoms, or a substituted or unsubstituted heterocyclic group having 5 to 50 ring atoms;
mx is 1, 2, 3, 4, or 5;
when two or more L₁₀₁ are present, two or more L₁₀₁ are mutually the same or different;
when two or more Ar₁₀₁ are present, two or more Ar₁₀₁ are mutually the same or different; and
* in the formula (11X) represents a bonding position to a benz[a]anthracene ring in the formula (1X), 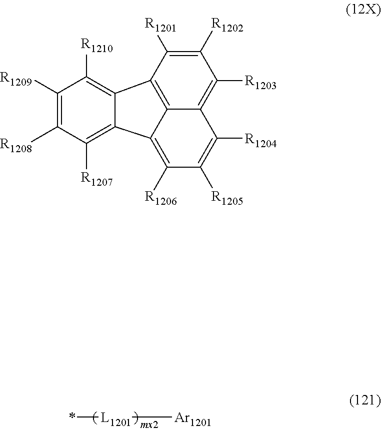
where, in the formula (12X):
at least one combination of adjacent two or more of R₁₂₀₁ to R₁₂₁₀ are mutually bonded to form a substituted or unsubstituted monocyclic ring, or mutually bonded to form a substituted or unsubstituted fused ring;
R₁₂₀₁ to R₁₂₁₀ neither forming the substituted or unsubstituted monocyclic ring nor forming the substituted or unsubstituted fused ring each independently represent a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (121);
a substituent for substituting the substituted or unsubstituted monocyclic ring, a substituent for substituting the substituted or unsubstituted fused ring, and at least one of R₁₂₀₁ to R₁₂₁₀ are a group represented by the formula (121);
when a plurality of groups represented by the formula (121) are present, the plurality of groups represented by the formula (121) are mutually the same or different;
L₁₂₀₁ is a single bond, a substituted or unsubstituted arylene group having 6 to 50 ring carbon atoms, or a substituted or unsubstituted divalent heterocyclic group having 5 to 50 ring atoms;
Ar₁₂₀₁ is a substituted or unsubstituted aryl group having 6 to 50 ring carbon atoms, or a substituted or unsubstituted heterocyclic group having 5 to 50 ring atoms;
mx2 is 0, 1, 2, 3, 4, or 5;
when two or more L₁₂₀₁ are present, the two or more L₁₂₀₁ are mutually the same or different;
when two or more Ar₁₂₀₁ are present, the two or more Ar₁₂₀₁ are mutually the same or different; and
* in the formula (121) represents a bonding position to a ring represented by the formula (12X), 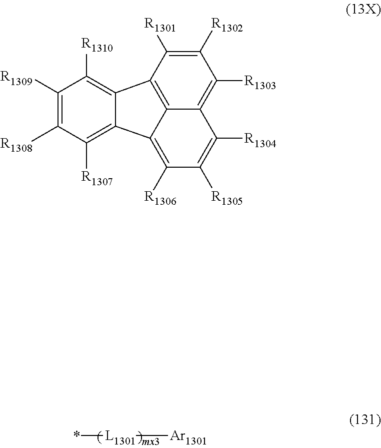
where, in the formula (13X):
R₁₃₀₁ to R₁₃₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (131);
at least one of R₁₃₀₁ to R₁₃₁₀ is a group represented by the formula (131);
when a plurality of groups represented by the formula (131) are present, the plurality of groups represented by the formula (131) are mutually the same or different;
L₁₃₀₁ is a single bond, a substituted or unsubstituted arylene group having 6 to 50 ring carbon atoms, or a substituted or unsubstituted divalent heterocyclic group having 5 to 50 ring atoms;
Ar₁₃₀₁ is a substituted or unsubstituted aryl group having 6 to 50 ring carbon atoms, or a substituted or unsubstituted heterocyclic group having 5 to 50 ring atoms;
mx3 is 0, 1, 2, 3, 4, or 5;
when two or more L₁₃₀₁ are present, the two or more L₁₃₀₁ are mutually the same or different;
when two or more Ar₁₃₀₁ are present, two or more Ar₁₃₀₁ are mutually the same or different; and
* in the formula (131) represents a bonding position to a fluoranthene ring in the formula (13X), 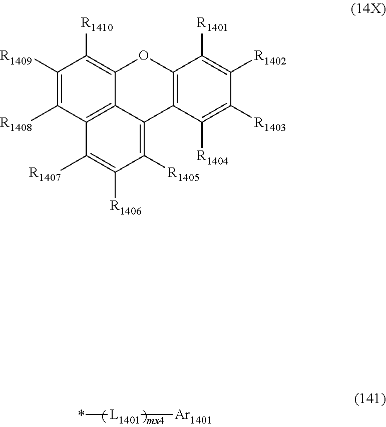
where, in the formula (14X):
R₁₄₀₁ to R₁₄₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (141);
at least one of R₁₄₀₁ to R₁₄₁₀ is a group represented by the formula (141);
when a plurality of groups represented by the formula (141) are present, the plurality of groups represented by the formula (141) are mutually the same or different;
L₁₄₀₁ is a single bond, a substituted or unsubstituted arylene group having 6 to 50 ring carbon atoms, or a substituted or unsubstituted divalent heterocyclic group having 5 to 50 ring atoms;
Ar₁₄₀₁ is a substituted or unsubstituted aryl group having 6 to 50 ring carbon atoms, or a substituted or unsubstituted heterocyclic group having 5 to 50 ring atoms;
mx4 is 0, 1, 2, 3, 4, or 5;
when two or more L₁₄₀₁ are present, the two or more L₁₄₀₁ are mutually the same or different;
when two or more Ar₁₄₀₁ are present, the two or more Ar₁₄₀₁ are mutually the same or different; and
* in the formula (141) represents a bonding position to a ring represented by the formula (14X), 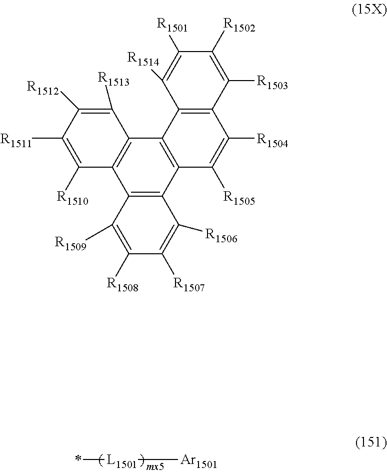
where, in the formula (15X):
R₁₅₀₁ to R₁₅₁₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (151);
at least one of R₁₅₀₁ to R₁₅₁₄ is a group represented by the formula (151);
when a plurality of group represented by the formula (151) are present, the plurality of groups represented by the formula (151) are mutually the same or different;
L₁₅₀₁ is a single bond, a substituted or unsubstituted arylene group having 6 to 50 ring carbon atoms, or a substituted or unsubstituted divalent heterocyclic group having 5 to 50 ring atoms;
Ar₁₅₀₁ is a substituted or unsubstituted aryl group having 6 to 50 ring carbon atoms, or a substituted or unsubstituted heterocyclic group having 5 to 50 ring atoms;
mx5 is 0, 1, 2, 3, 4, or 5;
when two or more L₁₅₀₁ are present, the two or more L₁₅₀₁ are mutually the same or different;
when two or more Ar₁₅₀₁ are present, the two or more Ar₁₅₀₁ are mutually the same or different; and
* in the formula (151) represents a bonding position to a ring represented by the formula (15X), 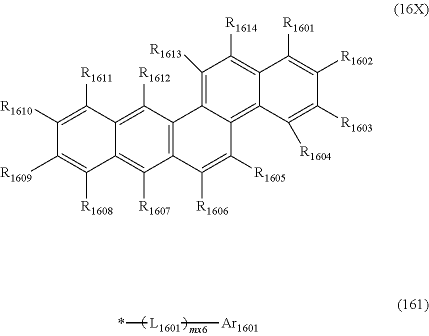
where, in the formula (16X):
R₁₆₀₁ to R₁₆₁₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (161);
at least one of R₁₆₀₁ to R₁₆₁₄ is a group represented by the formula (161);
when a plurality of group represented by the formula (161) are present, the plurality of groups represented by the formula (161) are mutually the same or different;
L₁₆₀₁ is a single bond, a substituted or unsubstituted arylene group having 6 to 50 ring carbon atoms, or a substituted or unsubstituted divalent heterocyclic group having 5 to 50 ring atoms;
Ar₁₆₀₁ is a substituted or unsubstituted aryl group having 6 to 50 ring carbon atoms, or a substituted or unsubstituted heterocyclic group having 5 to 50 ring atoms;
mx6 is 0, 1, 2, 3, 4, or 5;
when two or more L₁₆₀₁ are present, the two or more L₁₆₀₁ are mutually the same or different;
when two or more Ar₁₆₀₁ are present, the two or more Ar₁₆₀₁ are mutually the same or different; and
* in the formula (161) represents a bonding position to a ring represented by the formula (16X), and
in the first compound:
R₉₀₁, R₉₀₂, R₉₀₃, R₉₀₄, R₉₀₅, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

2. The organic electroluminescence device according to claim 1, wherein a singlet energy S₁(H1) of the first host material and a singlet energy S₁(D1) of the first dopant material satisfy a relationship of a numerical formula (Numerical Formula 2) below,
$${S}_{1} ( H 1 ) > {S}_{1} ( D 1 ) . \qquad ( Numerical Formula 2 )$$

3. The organic electroluminescence device according to claim 1, wherein
the first dopant material is not a complex.

4. The organic electroluminescence device according to claim 1, wherein
the first emitting layer does not contain a phosphorescent material.

5. The organic electroluminescence device according to claim 1, wherein
a singlet energy S₁(H2) of the second host material and a singlet energy S₁(D2) of the second dopant material satisfy a relationship of a numerical formula (Numerical Formula 4) below,
$${S}_{1} ( H 2 ) > {S}_{1} ( D 2 ) . \qquad ( Numerical Formula 4 )$$

6. The organic electroluminescence device according to claim 1, wherein
the second dopant material is not a complex.

7. The organic electroluminescence device according to claim 1, wherein
the second emitting layer does not contain a phosphorescent material.

8. The organic electroluminescence device according to claim 1, wherein
the triplet energy T₁(H1) of the first host material and the triplet energy T₁(H2) of the second host material satisfy a relationship of a numerical formula (Numerical Formula 5) below,
$${T}_{1} ( H 1 ) - {T}_{1} ( H 2 ) > 0.03 eV . \qquad ( Numerical Formula 5 )$$

9. The organic electroluminescence device according to claim 1, wherein
the first emitting layer and the second emitting layer are in direct contact with each other.

10. The organic electroluminescence device according to claim 1, wherein
the second host material is a second compound represented by the formula (2) below, 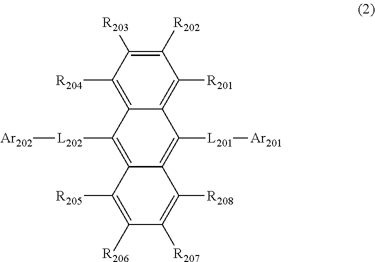
where, in the formula (2):
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms; and
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms, and
in the second compound:
R₉₀₁, R₉₀₂, R₉₀₃, R₉₀₄, R₉₀₅, R₉₀₆, R₉₀₇, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

11. An organic electroluminescence device comprising:
an anode;
a cathode;
a first emitting layer; and
a second emitting layer, wherein
the first emitting layer is provided between the anode and the cathode,
the second emitting layer is provided between the first emitting layer and the cathode,
the first emitting layer includes a first host material and a first dopant material,
the second emitting layer includes a second host material and a second dopant material,
the first host material and the second host material are mutually different,
the first dopant material is a compound that emits fluorescence having a main peak wavelength of 500 nm or less,
the second dopant material is a compound that emits fluorescence having a main peak wavelength of 500 nm or less,
the compound included in the first emitting layer and that emits fluorescence having a main peak wavelength of 500 nm or less is the same as or different from the compound included in the second emitting layer and that emits fluorescence having a main peak wavelength of 500 nm or less,
the first host material has, in a molecule, a biphenyl structure in which a first benzene ring and a second benzene ring are linked to each other with a single bond,
the first benzene ring and the second benzene ring in the biphenyl structure are linked to each other by cross-linking at two sites other than the single bond, the cross-linking including no double bond at one of the two sites, and the cross-linking including a double bond at the other of the two sites,
a triplet energy T₁(H1) of the first host material and a triplet energy T₁(H2) of the second host material satisfy a relationship of a numerical formula (Numerical Formula 1) below, and
the triplet energy T₁(H1) of the first host material and a triplet energy T₁(D1) of the first dopant material satisfy a relationship of a numerical formula (Numerical Formula 2A) below, a triplet energy T₁(D2) of the second dopant material and the triplet energy T₁(H2) of the second host material satisfy a relationship of a numerical formula (Numerical Formula 3) below, or both the relationship of the numerical formula (Numerical Formula 2A) and the relationship of the numerical formula (Numerical Formula 3) are satisfied,
$${T}_{1} ( H 1 ) > {T}_{1} ( H 2 ) \qquad ( Numerical Formula 1 )$$
$${T}_{1} ( D 1 ) > {T}_{1} ( H 1 ) \qquad ( Numerical Formula 2 A )$$
$${T}_{1} ( D 2 ) > {T}_{1} ( H 2 ) . \qquad ( Numerical Formula 3 )$$

12. The organic electroluminescence device according to claim 11, wherein
a singlet energy S₁(H1) of the first host material and a singlet energy S₁(D1) of the first dopant material satisfy a relationship of a numerical formula (Numerical Formula 2) below,
$${S}_{1} ( H 1 ) > {S}_{1} ( D 1 ) . \qquad ( Numerical Formula 2 )$$

13. The organic electroluminescence device according to claim 11, wherein
the first dopant material is not a complex.

14. The organic electroluminescence device according to claim 11, wherein
the first emitting layer does not contain a phosphorescent material.

15. The organic electroluminescence device according to claim 11, wherein
a singlet energy S₁(H2) of the second host material and a singlet energy S₁(D2) of the second dopant material satisfy a relationship of a numerical formula (Numerical Formula 4) below,
$${S}_{1} ( H 2 ) > {S}_{1} ( D 2 ) . \qquad ( Numerical Formula 4 )$$

16. The organic electroluminescence device according to claim 11, wherein
the second dopant material is not a complex.

17. The organic electroluminescence device according to claim 11, wherein
the second emitting layer does not contain a phosphorescent material.

18. The organic electroluminescence device according to claim 11, wherein
the triplet energy T₁(H1) of the first host material and the triplet energy T₁(H2) of the second host material satisfy a relationship of a numerical formula (Numerical Formula 5) below,
$${T}_{1} ( H 1 ) - {T}_{1} ( H 2 ) > 0.03 eV . \qquad ( Numerical Formula 5 )$$

19. The organic electroluminescence device according to claim 11, wherein
the first emitting layer and the second emitting layer are in direct contact with each other.

20. The organic electroluminescence device according to claim 11, wherein
the second host material is a second compound represented by the formula (2) below, 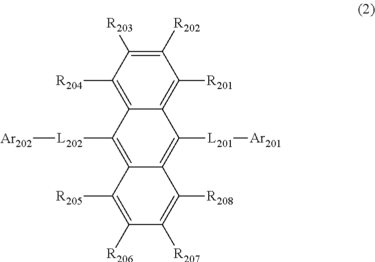
where, in the formula (2):
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms; and
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms, and
in the second compound:
R₉₀₁, R₉₀₂, R₉₀₃, R₉₀₄, R₉₀₅, R₉₀₆, R₉₀₇, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.
